(19) United States
(12) Patent Application Publication
(10) Pub. No.: US 2025/0051766 A1
(43) Pub. Date: Feb. 13, 2025

(54) TREATMENT OF NEUROLOGICAL DISEASES USING MODULATORS OF UNC13A GENE TRANSCRIPTS

(71) Applicants: sandra HINCKLEY, Cambridge, MA (US); Duncan BROWN, Berkeley, CA (US); Daniel ELBAUM, Cambridge, MA (US); QURALIS CORPORATION, Cambridge, MA (US)

(72) Inventors: SANDRA HINCKLEY, Cambridge, MA (US); DUNCAN BROWN, Berkeley, CA (US); DANIEL ELBAUM, Cambridge, MA (US); MARISA ELIZABETH KAMELGARN, Cambridge, MA (US)

(21) Appl. No.: 18/715,644

(22) PCT Filed: Dec. 2, 2022
(86) PCT No.: PCT/US2022/051713

§ 371 Date: Jun. 3, 2024

Related U.S. Application Data
(60) Provisional application No. 63/285,786, filed on Dec. 3, 2021.
(60) Provisional application No. 63/350,206, filed on Jun. 8, 2022.
(60) Provisional application No. 63/398,987, filed on Aug. 18, 2022.

(51) Int. Cl. C12N 15/113; A61K 45/06; A61P 25/28

(52) U.S. Cl. CPC C12N 15/113; A61K 45/06; A61P 25/28; C12N 2310/11; C12N 2310/31; C12N 2310/315; C12N 2310/321; C12N 2310/3231; C12N 2310/3341; C12N 2310/351

(57) ABSTRACT

Disclosed herein are UNC13A oligonucleotides with one or more spacers or without a spacer. In various embodiments, UNC13A oligonucleotides with spacer(s) reduce mis-spliced UNC13A transcripts and increase full length UNC13A transcripts, thereby imparting therapeutic efficacy against neurological diseases such as amyotrophic lateral sclerosis (ALS), frontotemporal dementia (FTD), or Alzheimer's disease (AD).

### CROSS-REFERENCE TO RELATED APPLICATIONS

This application claims the benefit of U.S. Provisional Application No. 63/285,786, filed on Dec. 3, 2021, U.S. Provisional Application No. 63/350,206, filed on Jun. 8, 2022, and U.S. Provisional Application No. 63/398,987, filed on Aug. 18, 2022, each of which is hereby incorporated herein by reference in its entirety for all purposes.

### FIELD OF THE DISCLOSURE

This application relates generally to methods of treating neurological diseases with UNC13A splice-switching antisense oligonucleotides, in particular, UNC13A antisense oligonucleotides with one or more spacers that target an UNC13A transcript.

### BACKGROUND

Motor neuron diseases are a class of neurological diseases that result in the degeneration and death of motor neurons—those neurons which coordinate voluntary movement of muscles by the brain. Motor neuron diseases may be sporadic or inherited, and may affect upper motor neurons and/or lower motor neurons. Motor neuron diseases include amyotrophic lateral sclerosis, progressive bulbar palsy, pseudobulbar palsy, primary lateral sclerosis, progressive muscular atrophy, spinal muscular atrophy, and post-polio syndrome.

Amyotrophic lateral sclerosis (ALS) is a group of motor neuron diseases affecting about 15,000 individuals in the United States of America. ALS is characterized by degeneration and death of upper and lower motor neurons, resulting in loss of voluntary muscle control. Motor neuron death is accompanied by muscle fasciculation and atrophy. Early symptoms of ALS include muscle cramps, muscle spasticity, muscle weakness (for example, affecting an arm, a leg, neck, or diaphragm), slurred and nasal speech, and difficulty chewing or swallowing. Loss of strength and control over movements, including those necessary for speech, eating, and breathing, eventually occur. Disease progression may be accompanied by weight loss, malnourishment, anxiety, depression, increased risk of pneumonia, muscle cramps, neuropathy, and possibly dementia. Most individuals diagnosed with ALS die of respiratory failure within five years of the first appearance of symptoms. Currently, there is no effective treatment for ALS.

ALS occurs in individuals of all ages, but is most common in individuals between 55 to 75 years of age, with a slightly higher incidence in males. ALS can be characterized as sporadic or familial. Sporadic ALS appears to occur at random and accounts for more than 90% of all incidences of ALS. Familial ALS accounts for 5-10% of all incidences of ALS.

FTD refers to a spectrum of progressive neurodegenerative diseases caused by loss of neurons in frontal and temporal lobes of the brain. FTD is the third most common form of dementia (following Alzheimer's disease and dementia with Lewy bodies), and the second most common form of dementia in individuals below 65 years of age. FTD is estimated to affect 50,000 to 60,000 individuals in the United States of America. FTD is characterized by changes in behavior and personality, and language dysfunction. Forms of FTD include behavioral variant FTD (bvFTD), semantic variant primary progressive aphasia (svPPA), and nonfluent variant primary progressive aphasia (nfvPPA). ALS with FTD is characterized by symptoms associated with FTD, along with symptoms of ALS such as muscle weakness, atrophy, fasciculation, spasticity, speech impairment (dysarthria), and inability to swallow (dysphagia). Individuals usually succumb to FTD within 5 to 10 years, while ALS with FTD often results in death within 2 to 3 years of the first disease symptoms appearing.

Like ALS, there is no known cure for FTD, or ALS with FTD, nor a therapeutic known to prevent or retard either disease's progression.

Thus, there is a pressing need to identify compounds and/or compositions capable of preventing, ameliorating, and treating neurological diseases such as: amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)), epilepsy, Cerebral Age-Related TDP-43 With Sclerosis (CARTS), facial onset sensory and motor neuronopathy, Guam Parkinson-dementia complex, multisystem proteinopathy, CTE, and synaptic diseases like autism.

### SUMMARY

Described herein are oligonucleotides comprising one or more spacers and comprising a sequence that is at least 85% complementary to an equal length portion of a UNC13A transcript. In one aspect, the present disclosure provides UNC13A oligonucleotides that target a UNC13A transcript (for example, a mis-spliced UNC13A transcript). In various embodiments, the oligonucleotides target a transcript for the treatment of neurological diseases, including motor neuron diseases, and/or neuropathies. For example, UNC13A oligonucleotides can be used to treat PD, ALS, FTD, ALS with FTD, and AD.

In one aspect the present disclosure provides a compound comprising a modified oligonucleotide comprising a sequence that is at least 85% complementary to an equal length portion of any one of SEQ ID NO: 5057-5065 or SEQ ID NOs: 5206-5208, a sequence having 90% identity thereof, or to a 15 to 50 contiguous nucleobase portion thereof, wherein at least one (i.e., one or more) nucleoside linkage of the oligonucleotide is a non-natural linkage. In various embodiments, the oligonucleotide comprises a spacer.

In one aspect the present disclosure provides a compound comprising a modified oligonucleotide comprising a sequence that is at least 85% complementary to an equal length portion of any one of SEQ ID NO: 5057-5065 or SEQ ID NOs: 5206-5208, a sequence having 90% identity thereof, or to a 15 to 50 contiguous nucleobase portion thereof, wherein at least one (i.e., one or more) nucleoside linkage of the oligonucleotide is a non-natural linkage, and further wherein the oligonucleotide comprises a spacer. In various embodiments, the oligonucleotide comprises a segment with at most 11 linked nucleosides. In various embodiments, the oligonucleotide comprises a segment with at most 10, 9, or 8 linked nucleosides. In various embodiments, the oligonucleotide comprises a segment with at most 7 linked nucleosides. In certain embodiments, the oligonucleotide comprises a segment with at most 6, 5, 4, 3, or 2 linked nucleosides. In certain embodiments, every segment of the oligonucleotide comprises at most 7 linked nucleosides.

In various embodiments, the oligonucleotide comprises a sequence that shares at least 85% identity with an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292. In various embodiments, the oligonucleotide comprises a sequence that shares at least 90% identity with an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292. In various embodiments, the oligonucleotide comprises a sequence that shares 95% identity with an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292. In various embodiments, the oligonucleotide comprises a sequence that shares 100% identity with an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292.

In various embodiments, the oligonucleotide comprises a segment with at most 11 linked nucleosides or at most 7 linked nucleosides, and wherein the oligonucleotide comprises a sequence that shares at least 85% identity with an equal length portion of any one of SEQ ID NO: 5057-5065 or SEQ ID NOs: 5206-5208. In various embodiments, the oligonucleotide comprises a segment with at most 6, 5, 4, 3, or 2 linked nucleosides, and wherein the oligonucleotide comprises a sequence that shares at least 85% identity with an equal length portion of any one of SEQ ID NO: 5057-5065 or SEQ ID NOs: 5206-5208. In various embodiments, the oligonucleotide comprises a segment with at most 6, 5, 4, 3, or 2 linked nucleosides, and wherein the oligonucleotide comprises a sequence that shares at least 90% identity with an equal length portion of any one of SEQ ID NO: 5057-5065 or SEQ ID NOs: 5206-5208.

In various embodiments, the oligonucleotide is at least 18, at least 19, at least 20, at least 21, at least 22, at least 23, at least 24, or at least 25 oligonucleotide units in length. In various embodiments, the oligonucleotide is at least 19 oligonucleotide units in length. In various embodiments, the spacer is a nucleoside-replacement group comprising a non-sugar substitute that is incapable of linking to a nucleotide base.

In various embodiments, the spacer is located between positions 10 and 15 of the oligonucleotide. In various embodiments, the spacer is located between positions 7 and 11 of the oligonucleotide. In various embodiments, the oligonucleotide further comprises a second spacer, wherein the second spacer is located between positions 14 and 22 of the oligonucleotide. In various embodiments, the spacer and the second spacer are separated by at least 5 nucleobases, at least 6 nucleobases, or at least 7 nucleobases in the oligonucleotide. In various embodiments, the spacer is located between positions 7 and 9 of the oligonucleotide, and wherein the second spacer is located between positions 15 and 18 of the oligonucleotide. In various embodiments, the spacer is located at position 8 of the oligonucleotide, and wherein the second spacer is located at position 16 of the oligonucleotide. In various embodiments, the oligonucleotide further comprises a third spacer, wherein the third spacer is located between positions 21 and 24 of the oligonucleotide.

In various embodiments, the spacer is located between positions 2 and 5 of the oligonucleotide. In various embodiments, the oligonucleotide further comprises a second spacer, wherein the second spacer is located between positions 8 and 12 of the oligonucleotide. In various embodiments, the oligonucleotide further comprises a third spacer, wherein the third spacer is located between positions 18 and 22 of the oligonucleotide. In various embodiments, the oligonucleotide further comprises a second spacer and a third spacer, wherein the three spacers are located at positions in the oligonucleotide such that each segment of the oligonucleotide has at most 7 linked nucleosides. In various embodiments, at least two of the three spacers are adjacent to a guanine nucleobase. In various embodiments, each of the at least two of the three spacers immediately precede a guanine nucleobase.

In various embodiments, each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

In certain embodiments, each of the first, second or third spacers is independently represented by Formula (X), wherein:

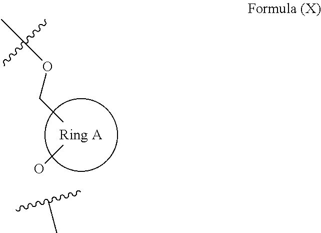

Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the

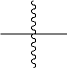

symbol represents the point of connection to an internucleoside linkage.

In various embodiments, each of the first, second or third spacers is independently represented by Formula (Xa), wherein:

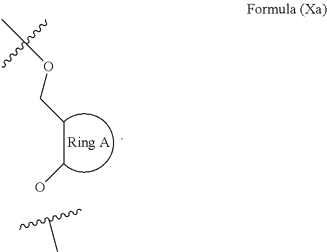

In some embodiments, ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrolidinyl, piperidinyl, piperazinyl, morpholinyl and azepanyl.

In further embodiments, ring A is tetrahydrofuranyl.

In other embodiments, ring A is tetrahydropyranyl.

In various embodiments, each of the first, second or third spacers is independently represented by Formula I, wherein:

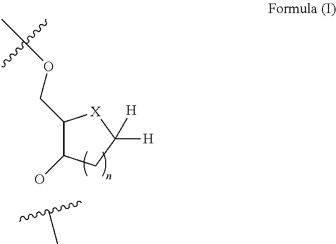

X is selected from —CH₂— and —O—; and
n is 0, 1, 2 or 3.

In various embodiments, each of the first, second or third spacers is independently represented by Formula I′, wherein:

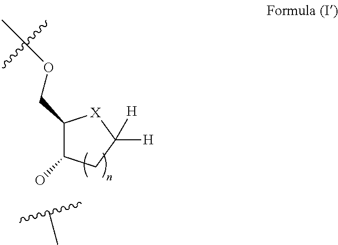

X is selected from —CH₂— and —O—; and
n is 0, 1, 2 or 3.

In various embodiments, each of the first, second or third spacers is independently represented by Formula (Ia), wherein:

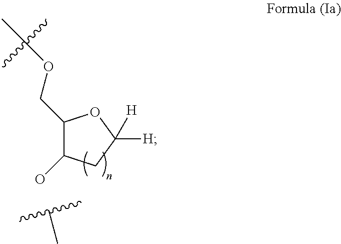

and
n is 0, 1, 2 or 3.

In various embodiments, each of the first, second or third spacers is independently represented by Formula (Ia′), wherein:

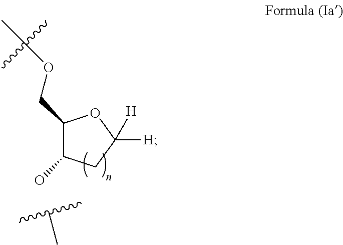

and
n is 0, 1, 2 or 3.

In certain embodiments, each of the first, second or third spacers is independently represented by Formula II, wherein:

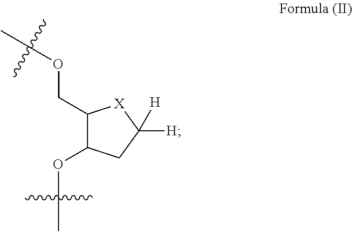

and
X is selected from —CH₂— and —O—.

In further embodiments, each of the first, second or third spacers is independently represented by Formula II′, wherein:

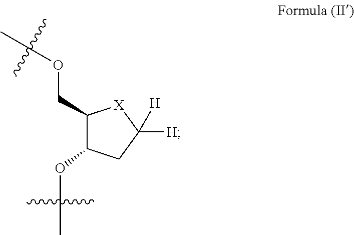

and
X is selected from —CH₂— and —O—.

In various embodiments, each of the first, second or third spacers is independently represented by Formula (Iia), wherein:

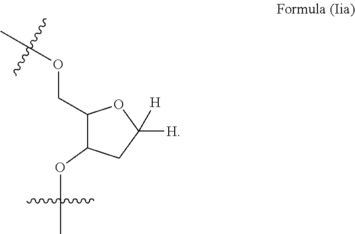

In further embodiments, each of the first, second or third spacers is independently represented by Formula (Iia′), wherein:

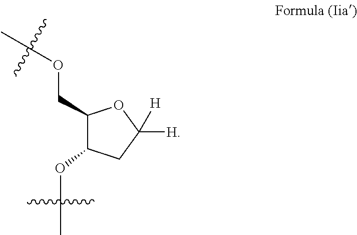

In some embodiments, the spacer is represented by Formula (IIi), wherein:

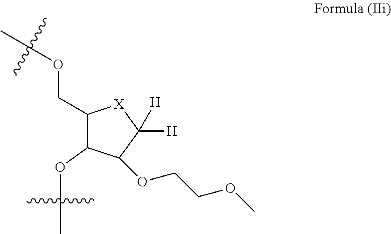

X is selected from —CH₂— and —O—.

In some embodiments, the spacer is represented by Formula (IIi′), wherein:

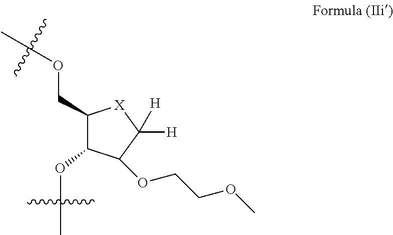

X is selected from —CH₂— and —O.

In some embodiments, the spacer is represented by Formula (IIib), wherein:

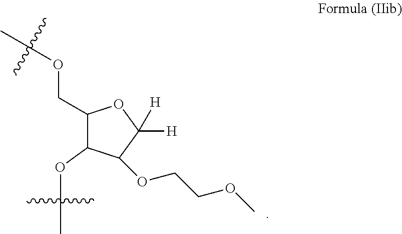 [SMILES: *OCC1OCC(OCCOC)C1O*]

In some embodiments, the spacer is represented by Formula (Iiib′), wherein:

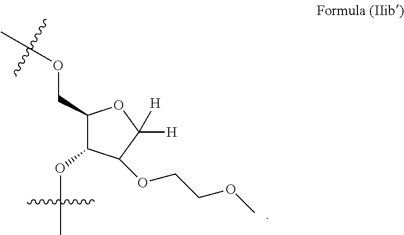 [SMILES: *OC[C@H]1OCC(OCCOC)[C@@H]1O*]

In various embodiments, each of the first, second or third spacers is independently represented by Formula III, wherein:

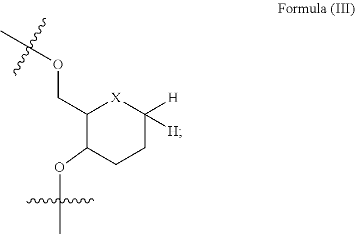

and
X is selected from —CH₂— and —O—.

In further embodiments, each of the first, second or third spacers is independently represented by Formula III′, wherein:

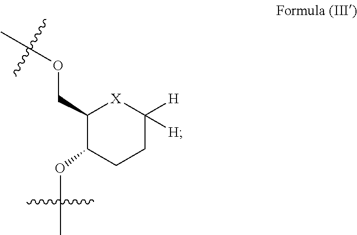

and
X is selected from —CH₂— and —O—.

In some embodiments, each of the first, second or third spacers is independently represented by Formula (IIIa), wherein:

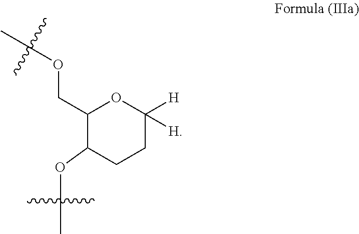

In further embodiments, each of the first, second or third spacers is independently represented by Formula (IIIa′), wherein:

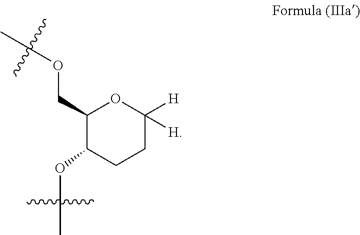

In various embodiments, the oligonucleotide comprising the spacer has a GC content of at least 10%. In various embodiments, the oligonucleotide comprising the spacer has a GC content of at least 20%. In various embodiments, the oligonucleotide comprising the spacer has a GC content of at least 25%. In various embodiments, the oligonucleotide comprising the spacer has a GC content of at least 30%. In various embodiments, the oligonucleotide comprising the spacer has a GC content of at least 40%. In various embodiments, the oligonucleotide comprising the spacer has a GC content of at least 50%.

In various embodiments, the oligonucleotide is between 12 and 40 oligonucleotide units in length.

In various embodiments, at least one (i.e., one or more) nucleoside linkage of the oligonucleotide is independently selected from the group consisting of a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, one or more nucleoside linkages that link a base at position 3 or position 4 of the oligonucleotide are phosphodiester linkages. In various embodiments, only one nucleoside linkage that links a base at position 3 or position 4 of the oligonucleotide is a phosphodiester linkage. In various embodiments, nucleoside linkages that link bases at both position 3 and position 4 of the oligonucleotide are phosphodiester linkages. In various embodiments, one or more bases immediately preceding a spacer in the oligonucleotide are linked through phosphodiester bonds. In various embodiments, only the base immediately preceding the spacer in the oligonucleotide is linked to the spacer through a phosphodiester bond. In various embodiments, the base immediately preceding the spacer in the oligonucleotide is further linked to a further preceding base through a phosphodiester bond. In various embodiments, the oligonucleotide comprises a second spacer, wherein a base immediately preceding the second spacer is linked to a further preceding base through a phosphodiester bond.

In various embodiments, one or more bases immediately succeeding a spacer in the oligonucleotide are linked through phosphodiester bonds. In various embodiments, only the base immediately succeeding the spacer in the oligonucleotide is linked to the spacer through a phosphodiester bond. In various embodiments, two bases immediately preceding the spacer in the oligonucleotide are linked through phosphodiester bonds. In various embodiments, one or more bases immediately preceding a spacer in the oligonucleotide are linked through phosphodiester bonds and wherein one or more bases immediately succeeding the spacer in the oligonucleotide are linked through phosphodiester bonds. In various embodiments, one base immediately preceding the spacer and one base immediately succeeding the spacer are linked through phosphodiester bonds. In various embodiments, the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds. In various embodiments, one base immediately preceding the second spacer and one base immediately succeeding the second spacer are linked through phosphodiester bonds. In various embodiments, the oligonucleotide comprises a range of bases that are linked through phosphodiester bonds, the range of bases comprising at least two bases. In various embodiments, the oligonucleotide comprises a range of bases that are linked through phosphodiester bonds, the range of bases comprising at least five bases. In various embodiments, the oligonucleotide comprises two or more spacers, and wherein the range of bases are positioned between the at least two spacers.

Additionally disclosed herein is a compound comprising an oligonucleotide comprising a nucleobase sequence that shares at least 90% identity to an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292. Additionally disclosed herein is an oligonucleotide comprising a nucleobase sequence that shares at least 90% identity to an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292. In various embodiments, the nucleobase sequence shares at least 95% identity to an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292. In various embodiments, the nucleobase sequence shares at least 100% identity to an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292. In various embodiments, the oligonucleotide is any one of a 19mer, 21mer, 23mer, or 25mer.

In various embodiments, an internucleoside linkage of the oligonucleotide is a modified internucleoside linkage. In various embodiments, the modified internucleoside linkage of the oligonucleotide is a phosphorothioate linkage. In various embodiments, all internucleoside linkages of the oligonucleotide are phosphorothioate linkages. In various embodiments, the phosphorothioate linkage is in one of a Rp configuration or a Sp configuration. In various embodiments, the oligonucleotide comprises at least one modified sugar moiety. In various embodiments, the modified sugar moiety is one of a 2′-OMe modified sugar moiety, bicyclic sugar moiety, 2′-O-(2-methoxyethyl) (MOE), 2′-deoxy-2′-fluoro nucleoside, 2′-fluoro-β-D-arabinonucleoside, locked nucleic acid (LNA), a tricyclic nucleic acid (tcDNA) (e.g., tricyclic nucleic acid with ethyl (2′O—CH₂—CH₂-4′C) as the bridge or tricyclic nucleic acid with methyl substituted methyl (2′O—CH(CH₂)-4′C) bridge), constrained ethyl 2′-4′-bridged nucleic acid (cEt), S-cEt, tcDNA, hexitol nucleic acids (HNA), tricyclic analog (e.g., tcDNA), and unlocked nucleic acids.

In various embodiments, the oligonucleotide exhibits at least a 30%, 40%, 5%, 60%, 70%, 80%, or 90% increase of full length UNC13A protein. In various embodiments, the oligonucleotide exhibits at least a 100% increase of full length UNC13A protein. In various embodiments, the oligonucleotide exhibits at least a 200% increase of full length UNC13A protein. In various embodiments, the oligonucleotide exhibits at least a 300% increase of full length UNC13A protein. In various embodiments, the oligonucleotide exhibits at least a 400% increase of full length UNC13A protein. In various embodiments, increase of the full length UNC13A protein is measured in comparison to a reduced level of full length UNC13A protein achieved using a TDP43 antisense oligonucleotide. In various embodiments, the oligonucleotide exhibits at least a 10%, 20%, 30%, 40%, 50%, 60%, 70%, 80%, 90%, or 100% rescue of full length UNC13A protein. In various embodiments, the oligonucleotide exhibits at least a 50%, 60%, 70%, 80%, or 90% reduction of a mis-spliced UNC13A transcript.

Additionally disclosed is a method of treating a neurological disease and/or a neuropathy in a patient in need thereof, the method comprising administering to the patient an oligonucleotide of any of the oligonucleotides disclosed above. In various embodiments, the neurological disease selected from the group consisting of: amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)), epilepsy, Cerebral Age-Related TDP-43 With Sclerosis (CARTS), facial onset sensory and motor neuronopathy, Guam Parkinson-dementia complex, multisystem proteinopathy, CTE, and synaptic diseases like autism. In various embodiments, the neurological disease is ALS. In various embodiments, the neurological disease is FTD. In various embodiments, the neurological disease is ALS with FTD. In various embodiments, the neurological disease is AD. In various embodiments, the neurological disease is PD. In various embodiments, the neuropathy is chemotherapy induced neuropathy.

Additionally disclosed is a method of restoring axonal outgrowth and/or regeneration of a neuron, the method comprising exposing the motor neuron to an oligonucleotide of any of the oligonucleotides disclosed above. Additionally disclosed is a method of increasing, promoting, stabilizing, or maintaining UNC13A expression and/or function in a neuron, the method comprising exposing the cell to an oligonucleotide of any of the oligonucleotides disclosed above.

In various embodiments, the neuron is a neuron of a patient in need of treatment of a neurological disease and/or a neuropathy. In various embodiments, the neuropathy is chemotherapy induced neuropathy. In various embodiments, the exposing is performed in vivo or ex vivo. In various embodiments, the exposing comprises administering the oligonucleotide to a patient in need thereof. In various embodiments, the oligonucleotide is administered topically, parenterally, intrathecally, intrathalamically, intracistemally, orally, rectally, buccally, sublingually, vaginally, pulmonarily, intratracheally, intranasally, transdermally, or intraduodenally. In various embodiments, the oligonucleotide is administered orally. In various embodiments, a therapeutically effective amount of the oligonucleotide is administered intrathecally, intrathalamically or intracisternally. In various embodiments, the patient is a human.

Additionally disclosed herein is a pharmaceutical composition comprising the oligonucleotide of any one of the oligonucleotides disclosed above, or a pharmaceutically acceptable salt thereof, and a pharmaceutically acceptable excipient. In various embodiments, the pharmaceutical composition is suitable for topical, intrathecal, intrathalamic, intracisternal, intracerebroventricular, parenteral, oral, pulmonary, intratracheal, intranasal, transdermal, rectal, buccal, sublingual, vaginal, or intraduodenal administration.

Additionally disclosed herein is a method of treating a neurological disease or a neuropathy in a patient in need thereof, the method comprising administering to a patient in need thereof a therapeutically effective amount of a pharmaceutical composition disclosed above. In various embodiments, the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism. In various embodiments, the neurological disease is ALS. In various embodiments, the neurological disease is FTD. In various embodiments, the neurological disease is ALS with FTD. In various embodiments, the neuropathy is chemotherapy induced neuropathy. In various embodiments, the pharmaceutical composition is administered topically, parenterally, orally, pulmonarily, rectally, buccally, sublingually, vaginally, intratracheally, intranasally, intracistemally, intrathecally, intrathalamically, intravenously, intramuscularly, transdermally, or intraduodenally. In various embodiments, wherein the pharmaceutical composition is administered intrathecally, intrathalamically intracerebroventricularly, or intracistemally. In various embodiments, a therapeutically effective amount of the oligonucleotide is administered intrathecally, intrathalamically or intracisternally. In various embodiments, the patient is human.

Additionally disclosed herein is a method for treating a neurological disease in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292, or a pharmaceutically acceptable salt thereof, wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

Additionally disclosed herein is a method for treating ALS in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

Additionally disclosed herein is a method for treating FTD in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

Additionally disclosed herein is a method for treating ALS with FTD in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, one or more nucleoside linkages that link a base at position 3 or position 4 of the oligonucleotide are phosphodiester linkages. In various embodiments, only one nucleoside linkage that links a base at position 3 or position 4 of the oligonucleotide is a phosphodiester linkage. In various embodiments, nucleoside linkages that link bases at both position 3 and position 4 of the oligonucleotide are phosphodiester linkages. In various embodiments, one or more bases immediately preceding a spacer in the oligonucleotide are linked through phosphodiester bonds. In various embodiments, only the base immediately preceding the spacer in the oligonucleotide is linked to the spacer through a phosphodiester bond. In various embodiments, the base immediately preceding the spacer in the oligonucleotide is further linked to a further preceding base through a phosphodiester bond. In various embodiments, the oligonucleotide comprises a second spacer, wherein a base immediately preceding the second spacer is linked to a further preceding base through a phosphodiester bond.

In various embodiments, one or more bases immediately succeeding a spacer in the oligonucleotide are linked through phosphodiester bonds. In various embodiments, only the base immediately succeeding the spacer in the oligonucleotide is linked to the spacer through a phosphodiester bond. In various embodiments, two bases immediately preceding the spacer in the oligonucleotide are linked through phosphodiester bonds. In various embodiments, one or more bases immediately preceding a spacer in the oligonucleotide are linked through phosphodiester bonds and wherein one or more bases immediately succeeding the spacer in the oligonucleotide are linked through phosphodiester bonds. In various embodiments, one base immediately preceding the spacer and one base immediately succeeding the spacer are linked through phosphodiester bonds. In various embodiments, the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds. In various embodiments, one base immediately preceding the second spacer and one base immediately succeeding the second spacer are linked through phosphodiester bonds. In various embodiments, the oligonucleotide comprises a range of bases that are linked through phosphodiester bonds, the range of bases comprising at least two bases. In various embodiments, the oligonucleotide comprises a range of bases that are linked through phosphodiester bonds, the range of bases comprising at least five bases. In various embodiments, the oligonucleotide comprises two or more spacers, and wherein the range of bases are positioned between the at least two spacers. In various embodiments, the oligonucleotide is any one of a 19mer, 21mer, 23mer, or 25mer.

In various embodiments, at least one (i.e., one or more) internucleoside linkage of the oligonucleotide is a phosphorothioate linkage. In various embodiments, all internucleoside linkages of the oligonucleotide are phosphorothioate linkages.

Additionally disclosed herein is an oligonucleotide and a pharmaceutically acceptable excipient, the oligonucleotide comprising a sequence that is at least 85% complementary to an equal length portion of any one of SEQ ID NO: 5057-5065 or SEQ ID NOs: 5206-5208, a sequence having 90% identity thereof, or to a 15 to 50 contiguous nucleobase portion thereof, wherein the oligonucleotide comprises a spacer and wherein the oligonucleotide is capable of increasing, restoring, or stabilizing expression of the UNC13A mRNA capable of translation of a functional UNC13A and/or activity and/or function of UNC13A protein in a cell or a human patient of an immune-mediated demyelinating disease, and wherein the level of increase, restoration, or stabilization of expression and/or activity and/or function is sufficient for use of the oligonucleotide as a medicament for the treatment of the immune-mediated demyelinating disease.

In various embodiments, the oligonucleotide comprises one or more chiral centers and/or double bonds. In various embodiments, the oligonucleotide exist as stereoisomers selected from geometric isomers, enantiomers, and diastereomers.

Additionally disclosed herein is a method of treating a neurological disease and/or a neuropathy in a patient in need thereof, the method comprising administering to a patient in need thereof a therapeutically effective amount of a pharmaceutical composition disclosed above, in combination with a second therapeutic agent. In various embodiments, the second therapeutic agent is selected from Riluzole (Dilute), PrimeC (combination of celecoxib and ciprofloxacin), Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), pridopidine, dual AON intrathecal administration (e.g., B11B067, BIIB078, and B11B105), BIIB100, levodopa/carbidopa, dopaminergic agents (e.g., ropinirole, pramipexole, rotigotine), medroxyprosterone, KCNQ2/KCNQ3 openers (e.g., retigabine, XEN1101, QRL-101), anticonvulsants and psychostimulant agents, and/or a therapy (e.g., selected from breathing care, physical therapy, occupational therapy, speech therapy, nutritional support), for treating said neurologic disease.

Additionally disclosed herein is a method of treating a neurological disease and/or a neuropathy in a patient in need thereof, the method comprising administering to a patient in need thereof a therapeutically effective amount of a pharmaceutical composition disclosed above, wherein at least one (i.e., one or more) nucleoside linkage of the oligonucleotide is a non-natural linkage, wherein the oligonucleotide comprises a spacer, and wherein the oligonucleotide further comprises a targeting or conjugate moiety selected from cholesterol, lipoic acid, panthothenic acid, polyethylene glycol, and an antibody for crossing the blood brain barrier.

In various embodiments, the spacer is a nucleoside-replacement group comprising a non-sugar substitute that is incapable of linking to a nucleotide base. In various embodiments, the spacer is located between positions 10 and 15 of the oligonucleotide. In various embodiments, the spacer is located between positions 7 and 11 of the oligonucleotide. In various embodiments, the oligonucleotide further comprises a second spacer, wherein the second spacer is located between positions 14 and 22 of the oligonucleotide. In various embodiments, the spacer and the second spacer are separated by at least 5 nucleobases, at least 6 nucleobases, or at least 7 nucleobases in the oligonucleotide. In various embodiments, the spacer is located between positions 7 and 9 of the oligonucleotide, and wherein the second spacer is located between positions 15 and 18 of the oligonucleotide. In various embodiments, the spacer is located at position 8 of the oligonucleotide, and wherein the second spacer is located at position 16 of the oligonucleotide.

In various embodiments, the oligonucleotide further comprises a third spacer, wherein the third spacer is located between positions 21 and 24 of the oligonucleotide. In various embodiments, the spacer is located between positions 2 and 5 of the oligonucleotide. In various embodiments, the oligonucleotide further comprises a second spacer, wherein the second spacer is located between positions 8 and 12 of the oligonucleotide. In various embodiments, the oligonucleotide further comprises a third spacer, wherein the third spacer is located between positions 18 and 22 of the oligonucleotide. In various embodiments, the oligonucleotide further comprises a second spacer and a third spacer, wherein the three spacers are located at positions in the oligonucleotide such that each segment of the oligonucleotide has at most 7 linked nucleosides.

In various embodiments, at least two of the three spacers are adjacent to a guanine nucleobase. In various embodiments, each of the at least two of the three spacers immediately precede a guanine nucleobase.

In various embodiments, of the methods described herein, each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

In certain embodiments, each of the first, second or third spacers is independently represented by Formula (X), wherein:

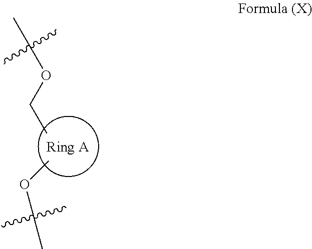

Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the

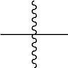

symbol represents the point of connection to an internucleoside linkage.

In various embodiments, each of the first, second or third spacers is independently represented by Formula (Xa), wherein:

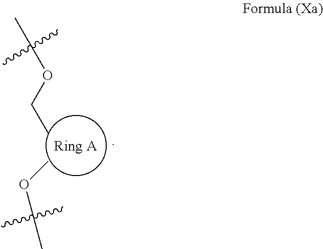

In some embodiments, ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrolidinyl, piperidinyl, piperazinyl, morpholinyl and azepanyl.

In further embodiments, ring A is tetrahydrofuranyl.

In other embodiments, ring A is tetrahydropyranyl.

In various embodiments, each of the first, second or third spacers is independently represented by Formula (I), wherein:

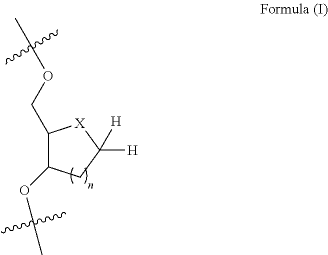

X is selected from —CH₂— and —O—; and
n is 0, 1, 2 or 3.

In various embodiments, the spacer or the second spacer is represented by Formula (I′), wherein:

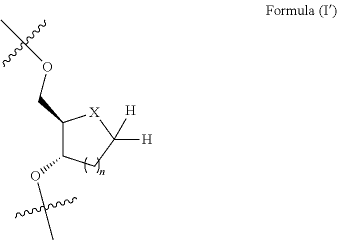

X is selected from —CH₂— and —O—; and
n is 0, 1, 2 or 3.

In various embodiments, each of the first, second or third spacers is independently represented by Formula (Ia), wherein:

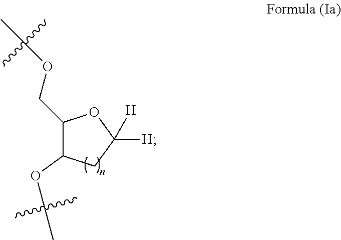

and
n is 0, 1, 2 or 3.

In various embodiments, each of the first, second or third spacers is independently represented by Formula (Ia′), wherein:

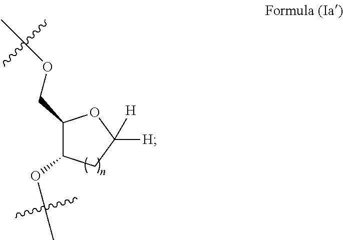

and
n is 0, 1, 2 or 3.

In certain embodiments, each of the first, second or third spacers is independently represented by Formula II, wherein:

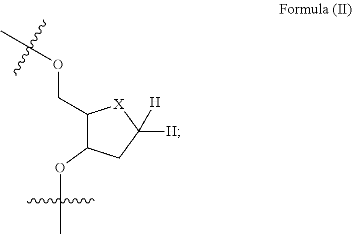

and
X is selected from —CH₂— and —O—.

In further embodiments, each of the first, second or third spacers is independently represented by Formula II′, wherein:

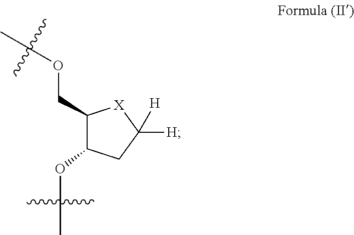

and
X is selected from —CH₂— and —O—.

In various embodiments, each of the first, second or third spacers is independently represented by Formula (Iia), wherein:

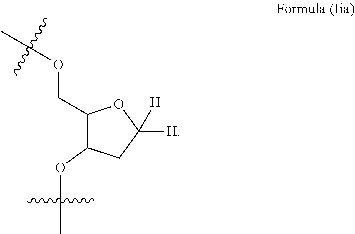

In further embodiments, each of the first, second or third spacers is independently represented by Formula (Iia′), wherein:

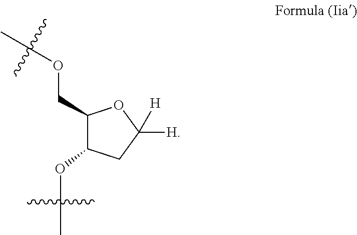

In some embodiments, the spacer is represented by Formula (IIi), wherein:

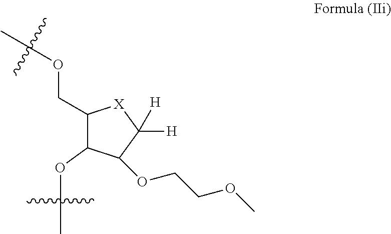

X is selected from —CH₂— and —O—.

In some embodiments, the spacer is represented by Formula (IIi′), wherein:

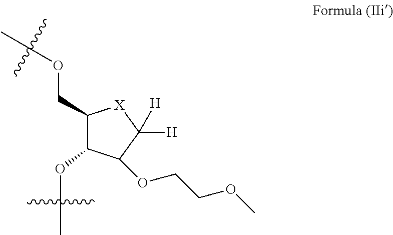

X is selected from —CH₂— and —O.

In some embodiments, the spacer is represented by Formula (IIib), wherein:

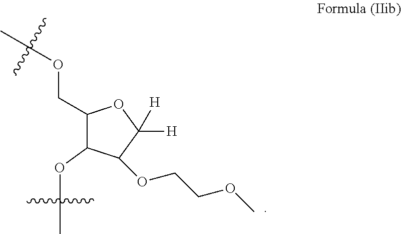 [SMILES: *OCC1OCC(OCCOC)C1O*]

In some embodiments, the spacer is represented by Formula (Iiib′), wherein:

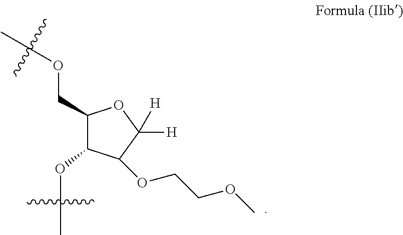 [SMILES: *OC[C@H]1OCC(OCCOC)[C@@H]1O*]

In various embodiments, each of the first, second or third spacers is independently represented by Formula III, wherein:

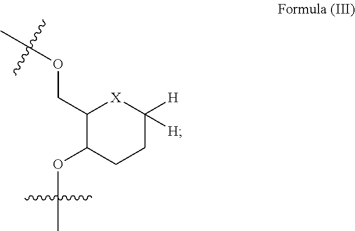

and
X is selected from —CH₂— and —O—.

In further embodiments, each of the first, second or third spacers is independently represented by Formula III′, wherein:

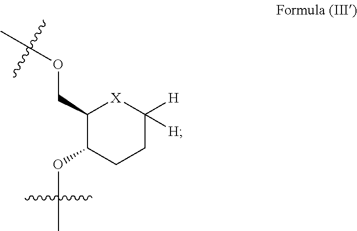

and
X is selected from —CH₂— and —O—.

In some embodiments, each of the first, second or third spacers is independently represented by Formula (IIIa), wherein:

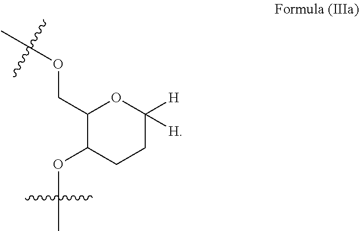

In further embodiments, each of the first, second or third spacers is independently represented by Formula (IIIa′), wherein:

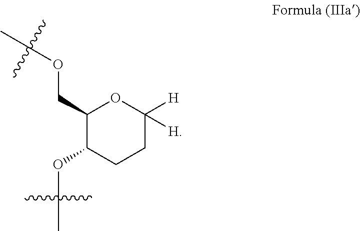

In various embodiments, the oligonucleotide comprising the spacer has a GC content of at least 10%. In various embodiments, the oligonucleotide comprising the spacer has a GC content of at least 20%. In various embodiments, the oligonucleotide comprising the spacer has a GC content of at least 25%. In various embodiments, the oligonucleotide comprising the spacer has a GC content of at least 30%. In various embodiments, the oligonucleotide comprising the spacer has a GC content of at least 40%. In various embodiments, the oligonucleotide comprising the spacer has a GC content of at least 50%.

### BRIEF DESCRIPTION OF THE DRAWINGS

FIG. 1 shows an example antisense oligonucleotide (AON), a portion of which is complementary to a mRNA transcript or pre-mRNA transcript. Dashed lines indicate positions of the AON which may or may not be occupied by a spacer.

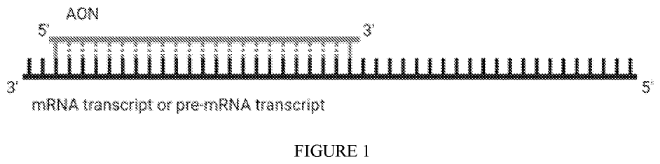

### DETAILED DESCRIPTION

The features and other details of the disclosure will now be more particularly described. Certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

Disclosed herein are oligonucleotides capable of targeting a region of a transcript transcribed from a gene. In various embodiments, such oligonucleotides target a UNC13A transcript. Additionally disclosed herein are oligonucleotides, including antisense oligonucleotide sequences, and methods for treating neurological diseases, such as amyotrophic lateral sclerosis and frontotemporal dementia, and/or neuropathies such as chemotherapy induced neuropathy, using same. In various embodiments, the oligonucleotides target a sequence of UNC13A transcripts resulting in the reduction of levels of mis-spliced UNC13A transcripts Also disclosed are pharmaceutical compositions comprising UNC13A oligonucleotides that target a region of UNC13A transcripts, for treating neurological diseases and/or neuropathies; and manufacture of medicaments containing a disclosed UNC13A oligonucleotide that targets a region of UNC13A transcripts to be used in treating a neurological disease and/or neuropathy.

### Definitions

The terms “treat,” “treatment,” “treating,” and the like are used herein to generally mean obtaining a desired pharmacological and/or physiological effect. The effect may be therapeutic in terms of partially or completely curing a disease and/or adverse effect attributed to the disease. The term “treatment” as used herein covers any treatment of a disease in a mammal, particularly a human, and includes: (a) inhibiting the disease, i.e., preventing the disease from increasing in severity or scope; (b) relieving the disease, i.e., causing partial or complete amelioration of the disease; or (c) preventing relapse of the disease, i.e., preventing the disease from returning to an active state following previous successful treatment of symptoms of the disease or treatment of the disease.

“Preventing” includes delaying the onset of clinical symptoms, complications, or biochemical indicia of the state, disorder, disease, or condition developing in a subject that may be afflicted with or predisposed to the state, disorder, disease, or condition but does not yet experience or display clinical or subclinical symptoms of the state, disorder, disease, or condition. “Preventing” includes prophylactically treating a state, disorder, disease, or condition in or developing in a subject, including prophylactically treating clinical symptoms, complications, or biochemical indicia of the state, disorder, disease, or condition in or developing in a subject.

The term “pharmaceutically acceptable carrier” or “pharmaceutically acceptable excipient” as used herein interchangeably refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one biologically active compound, for example, a UNC13A antisense oligonucleotide (AON), as disclosed herein formulated together with one or more pharmaceutically acceptable excipients.

“Individual,” “patient,” or “subject” are used interchangeably and include any animal, including mammals, preferably mice, rats, other rodents, rabbits, dogs, cats, swine, cattle, sheep, horses, or non-human primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, non-human primates, and the like). In some embodiments, the mammal treated in the methods of the invention is desirably a mammal in whom modulation of UNC13A expression and/or activity is desired.

As used herein, “UNC13A” (also known as Unc-13 Homolog A, Munc13-1, KIAA1032, unc-13 homolog A (C. elegans), or Protein Unc-13 Homolog A) refers to the gene or gene products (e.g., protein or mRNA transcript (including pre-mRNA) encoded by the gene) identified by Entrez Gene ID No. 23025 and allelic variants thereof, as well as orthologs found in non-human species (e.g., non-human primates or mice).

The term “UNC13A transcript” refers to a UNC13A transcript which can be a UNC13A pre-mRNA sequence or a UNC13A mature RNA sequence. UNC13A transcript sequences are shown to contain thymine (T), but one of skill in the art will appreciate that thymine (T) can generally be replaced with uracil (U) in RNA sequences.

The term “UNC13A oligonucleotide,” “UNC13A antisense oligonucleotide,” or “UNC13A AON” refers to an oligonucleotide that is capable of increasing, restoring, or stabilizing full-length UNC13A activity e.g., full length UNC13A expression, for example, full length UNC13A mRNA and/or full length UNC13A protein expression. Generally, a UNC13A oligonucleotide reduces the level of mis-spliced UNC13A transcripts by targeting a UNC13A transcript (e.g., UNC13A pre-mRNA or mis-spliced UNC13A with a target sequence). In various embodiments, a UNC13A oligonucleotide comprises a sequence that is at least 85% complementary to an equal length portion of a transcript comprising a sequence at least 90% identity to SEQ ID NO: 5057-5065 or SEQ ID NOs: 5206-5208, or a contiguous 15 to 50 nucleobase portion of SEQ ID NO: 5057-5065 or SEQ ID NOs: 5206-5208. UNC13A target sequences are shown to contain thymine (T), but one of skill in the art will appreciate that thymine (T) can generally be replaced with uracil (U) in RNA sequences.

In various embodiments, UNC13A oligonucleotides are characterized by having one or more spacers, where each spacer divides up the UNC13A oligonucleotide into segments of linked nucleosides. In various embodiments, UNC13A oligonucleotides have two spacers. In one embodiment, UNC13A oligonucleotides have two segments of linked nucleosides separated by one spacer. In one embodiment, UNC13A oligonucleotides have three segments of linked nucleosides separated by two spacers. In such embodiments, UNC13A oligonucleotides have one segment with at most 7 linked nucleosides. For example, a UNC13A oligonucleotide may have, from the 5′ to the 3′ end, 5 linked nucleosides, followed by a spacer, 10 linked nucleosides, followed by a second spacer, and 8 linked nucleosides. Thus, the first segment of 5 linked nucleosides satisfies the one segment with at most 7 linked nucleosides. In various embodiments, UNC13A oligonucleotides have three spacers that divide the UNC13A oligonucleotide into four segments. In various embodiments, each of the four segments of the UNC13A oligonucleotide have at most 7 linked nucleosides.

As used herein, the term “UNC13A oligonucleotide” encompasses a “UNC13A parent oligonucleotide,” a “UNC13A oligonucleotide with one or more spacers” (e.g., UNC13A oligonucleotide with two spacers or a UNC13A oligonucleotide with three spacers), a “UNC13A oligonucleotide variant with one or more spacers.” Examples of UNC13A oligonucleotides include oligonucleotides comprising a sequence of any one of SEQ ID NOs: 1-1264, SEQ ID NO: 2529-3792, SEQ ID NOs: 5066-5166, SEQ ID NOs: 5168-5202, SEQ ID NOs: 5209-5221, or SEQ ID NOs: 5235-5292.

The term “UNC13A parent oligonucleotide” refers to an oligonucleotide that targets a UNC13A transcript and is capable of increasing, restoring, or stabilizing full-length UNC13A activity e.g., full length UNC13A expression, for example, full length UNC13A mRNA and/or full length UNC13A protein expression. UNC13A parent oligonucleotides do not include a spacer. Examples of UNC13A parent oligonucleotides include oligonucleotides comprising a sequence of any one of SEQ ID NO: 1-1264. As described hereafter, UNC13A oligonucleotide with spacers and UNC13A oligonucleotide variants are described in relation to a corresponding UNC13A parent oligonucleotide.

The term “UNC13A oligonucleotide variant” refers to a UNC13A oligonucleotide that represents a modified version of a corresponding UNC13A parent oligonucleotide. For example, a UNC13A oligonucleotide variant represents a shortened version of a UNC13A parent oligonucleotide. In various embodiments, a UNC13A oligonucleotide variant is any one of a 15mer, 16mer, 17mer, 18mer 19mer, 20mer, 21mer, 22mer 23mer, 24mer, 25mer, 26mer, or 27mer. Examples of UNC13A oligonucleotide variants include oligonucleotides comprising a sequence of any one of SEQ ID NO: 2529-3792. In various embodiments, UNC13A oligonucleotide variants comprise one or more spacers. Such UNC13A oligonucleotide variants comprise a sequence of any one of SEQ ID NO: 5066-5166, SEQ ID NOs: 5168-5202, SEQ ID NOs: 5209-5221, or SEQ ID NOs: 5235-5292.

The term “oligonucleotide with one or more spacers” or “oligonucleotide comprising a spacer” refers to an oligonucleotide with at least one spacer. An oligonucleotide with one or more spacers can, in various embodiments, include one spacer, two spacers, three spacers, four spacer, five spacers, six spacers, seven spacers, eight spacers, nine spacers, or ten spacers. In various embodiments, an oligonucleotide comprising one or more spacers includes at least one segment with at most 7 linked nucleosides. For example, as described in a 5′ to 3′ direction, an oligonucleotide comprising a spacer can include a segment with 7 linked nucleosides, followed by a spacer, a second segment with 9 linked nucleosides, followed by a second spacer, and a third segment with 7 linked nucleosides. Here, the first segment of 7 linked nucleosides and the third segment of 7 linked nucleosides each represents segments with at most 7 linked nucleosides. As another example, an oligonucleotide comprising a spacer can include a segment with 10 linked nucleosides, followed by a spacer, a second segment with 10 linked nucleosides, followed by a second spacer, and a third segment with 3 linked nucleosides. Here, the third segment of 3 linked nucleosides represents the segment with at most 7 linked nucleosides. In various embodiments, an oligonucleotide with one or more spacers includes multiple segments with at most 7 linked nucleosides. In various embodiments, every segment of an oligonucleotide with one or more spacers has at most 7 linked nucleosides. For example, the oligonucleotide may be a 23mer and include two spacers that divide the 23mer into three separate segments of 7 linked nucleosides each. Therefore, each segment of the oligonucleotide has at most 7 linked nucleosides.

Generally, UNC13A oligonucleotides comprising one or more spacers are described in reference to a corresponding UNC13A parent oligonucleotide or a corresponding UNC13A oligonucleotide variant. Example UNC13A oligonucleotides comprising one or more spacers include any of SEQ ID NOs: 5066-5166, SEQ ID NOs: 5168-5202, SEQ ID NOs: 5209-5221, or SEQ ID NOs: 5235-5292.

In various embodiments, one or more spacers may be located at one or more positions of an oligonucleotide. A spacer may be located between a first position and a second position of the oligonucleotide. As used herein, a spacer located between a first position and second position encompasses the spacer being located at the first position, located at the second position, or located at any position of the oligonucleotide sandwiched by the first position and the second position.

In the present specification, the term “therapeutically effective amount” means the amount of an oligonucleotide that will elicit the biological or medical response of a tissue, system, animal or human that is being sought by the researcher, veterinarian, medical doctor, or other clinician. In one embodiment, the oligonucleotide comprises a sequence that is at least 85% complementary to an equal length portion of a transcript comprising a sequence at least 90% identity to SEQ ID NO: 5057-5065 or SEQ ID NOs: 5206-5208, or a contiguous 15 to 50 nucleobase portion of SEQ ID NO: 5057-5065 or SEQ ID NOs: 5206-5208. The oligonucleotide is administered in therapeutically effective amounts to treat and/or prevent a disease, condition, disorder, or state, for example, a neurological disease and/or a neuropathy. Alternatively, a therapeutically effective amount of an oligonucleotide is the quantity required to achieve a desired therapeutic and/or prophylactic effect, such as an amount which results in the prevention of or a decrease in the symptoms associated with a disease associated with reduced UNC13A activity in the motor neurons.

The phrase “a UNC13A oligonucleotide that targets a UNC13A transcript” refers to a UNC13A oligonucleotide that binds to a UNC13A transcript

The term “pharmaceutically acceptable salt(s)” as used herein refers to salts of acidic or basic groups that may be present in a UNC13A oligonucleotide used in the present compositions. A UNC13A oligonucleotide included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. A UNC13A oligonucleotide included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, and lithium salts. Pharmaceutically acceptable salts of the disclosure include, for example, pharmaceutically acceptable salts of UNC13A oligonucleotides that include a sequence of any of SEQ ID NOs: 1-1264, SEQ ID NOs: 2529-3792, SEQ ID NOs: 5066-5166, SEQ ID NOs: 5168-5202, SEQ ID NOs: 5209-5221, or SEQ ID NOs: 5235-5292.

A UNC13A oligonucleotide of the disclosure may contain one or more chiral centers, groups, linkages, and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S” (or “Rp” or “Sp”) depending on the configuration of substituents around the stereogenic atom, for example, a stereogenic carbon, phosphorous, or sulfur atom. In some embodiments, one or more linkages of the compound may have a Rp or Sp configuration (e.g., one or more phosphorothioate linkages have either a Rp or Sp configuration). The configuration of each phosphorothioate linkage may be independent of another phosphorothioate linkage (e.g., one phosphorothioate linkage has a Rp configuration and a second phosphorothioate linkage has a Sp configuration). In various embodiments, the UNC13A oligonucleotide can have a mixed configuration of phosphorothioate linkages. For example, the UNC13A oligonucleotide may have five phosphorothioate linkages in a Rp configuration, followed by fifteen phosphorothioate linkages in a Sp configuration, followed by five phosphorothioate linkages in a Rp configuration. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of a UNC13A oligonucleotide of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase super critical fluid chromatography, chiral-phase simulated moving bed chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

The UNC13A oligonucleotide disclosed herein can exist in solvated as well as unsolvated forms with pharmaceutically acceptable solvents such as water, ethanol, and the like, and it is intended that the invention embrace both solvated and unsolvated forms.

The disclosure also embraces fluorescently labeled compounds of the invention. The disclosure also embraces isotopically labeled compounds of the invention (i.e., isotopically labeled UNC13A oligonucleotide) which are identical to those recited herein, except that one or more atoms are replaced by an atom having an atomic mass or mass number different from the atomic mass or mass number abundantly found in nature. Examples of isotopes that can be incorporated into compounds of the invention include isotopes of hydrogen, carbon, nitrogen, oxygen, phosphorus, fluorine and chlorine, such as ²H, ³H, ¹¹C, ¹³C, ¹⁴C, ¹⁵N, ¹⁸O, ¹⁷O, ³¹P, ³²P, ³³P, ³⁵S, ¹⁸F, and ³⁶Cl, respectively.

Certain isotopically labeled disclosed compounds (e.g., those labeled with ³H, ¹⁴C, or ³⁵S) are useful in compound and/or substrate tissue distribution assays. Tritiated (i.e., ³H), carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances.

As used herein, “2′-O-(2-methoxyethyl)” (also 2′-MOE and 2′-O(CH₂)₂OCH₃ and MOE) refers to an O-methoxyethyl modification of the 2′ position of a furanose ring. A 2′-O-(2-methoxyethyl) is used interchangeably as “2′-O-methoxyethyl” in the present disclosure. A sugar moiety in a nucleoside modified with 2′-MOE is a modified sugar.

As used herein, “2′-MOE nucleoside” (also 2′-O-(2-methoxyethyl) nucleoside) means a nucleoside comprising a 2′-MOE modified sugar moiety.

As used herein, “2′-substituted nucleoside” means a nucleoside comprising a substituent at the 2′-position of the furanose ring other than H or OH. In certain embodiments, 2′ substituted nucleosides include nucleosides with bicyclic sugar modifications.

As used herein, “5-methyl cytosine” (5-MeC) means a cytosine modified with a methyl group attached to the 5 position. A 5-methyl cytosine (5-MeC) is a modified nucleobase.

As used herein, “bicyclic sugar” means a furanose ring modified by the bridging of two atoms. A bicyclic sugar is a modified sugar.

As used herein, “bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

As used herein, “cap structure” or “terminal cap moiety” means chemical modifications, which have been incorporated at either terminus of an antisense compound.

As used herein, “cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

As used herein, “constrained ethyl nucleoside” (also cEt nucleoside) means a nucleoside comprising a bicyclic sugar moiety comprising a 4′-CH(CH₃)—O-2′ bridge. In some embodiments, cEt can be modified. In some embodiments, the cEt can be S-cEt (in an S-constrained ethyl 2′-4′-bridged nucleic acid). In some other embodiments, the cEt can be R-cEt.

As used herein, “internucleoside linkage” refers to the covalent linkage between adjacent nucleosides in an oligonucleotide. In some embodiments, as used herein, “non-natural linkage” refers to a “modified internucleoside linkage.”

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence. As an example to the contrary, two nucleosides separated by a spacer are not contiguous.

As used herein, “locked nucleic acid” or “LNA” or “LNA nucleosides” means nucleic acid monomers having a bridge (e.g., methylene, ethylene, aminooxy, or oxyimino bridge) connecting two carbon atoms between the 4′ and 2′ position of the nucleoside sugar unit, thereby forming a bicyclic sugar. Examples of such bicyclic sugar include, but are not limited to (A) α-L-Methyleneoxy (4′—CH₂—O-2′) LNA, (B) β-D-Methyleneoxy (4′—CH₂—O-2′) LNA, (C) Ethyleneoxy (4′—(CH₂)₂-O-2′) LNA, (D) Aminooxy (4′—CH₂—O—N(R)-2′) LNA and (E) Oxyamino (4′—CH₂—N(R)—O-2′) LNA; wherein R is H, C₁-C₁₂ alkyl, or a protecting group (see U.S. Pat. No. 7,427,672, issued on Sep. 23, 2008).

As used herein, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′—CH₂-2′,4′—(CH₂)₂-2′,4′—(CH₂)₃-2′,4′—CH₂—O-2′,4′—(CH₂)₂—O-2′,4′—CH₂—O—N(R₁)—O-2′ and 4′—CH₂—N(R₁)—O-2′— bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a bridge to form the bicyclic sugar moiety. The bridge can be a methylene (—CH₂—) group connecting the 2′ oxygen atom and the 4′ carbon atom, for which the term methyleneoxy (4′—CH₂—O-2′) LNA is used. Furthermore, in the case of the bicyclic sugar moiety having an ethylene bridging group in this position, the term ethyleneoxy (4′-CH₂CH₂—O-2′) LNA is used.

As used herein, a “spacer” refers to a nucleoside-replacement group (e.g., a non-nucleoside group that replaces a nucleoside present in a UNC13A parent oligonucleotide). The spacer is characterized by the lack of a nucleotide base and by the replacement of the nucleoside sugar moiety with a non-sugar substitute. The non-sugar substitute group of a spacer lacks an aldehyde, ketone, acetal, ketal, hemiacetal or hemiketal group. The non-sugar substitute group of a spacer is thus capable of connecting to the 3′ and 5′ positions of the nucleosides adjacent to the spacer through an internucleoside linker as described herein, but not capable of forming a covalent bond with a nucleotide base (i.e., not capable of linking a nucleobase to another group, such as an internucleoside linkage, conjugate group, or terminal group in an oligonucleotide). Generally, a UNC13A oligonucleotide with a spacer is described in relation to a UNC13A parent oligonucleotide, wherein the spacer replaces a nucleoside of the UNC13A parent oligonucleotide. In all embodiments of the present disclosure, a spacer cannot hybridize to a nucleoside comprising a nucleobase at the corresponding position of a UNC13A transcript, within the numerical order of the length of the AON oligonucleotide (i.e., if the spacer is positioned after nucleoside 4 of an AON (i.e., at position 5 from the 5′-end), the spacer is not complementary to the nucleoside (A, C, G, or U) at the same corresponding position of the target UNC13A transcript)).

As used herein, “mismatch” or a “non-complementary group” refers to the case when a group (e.g., nucleobase) of a first nucleic acid is not capable of pairing with the corresponding group (e.g., nucleobase) of a second or target nucleic acid.

As used herein, “modified internucleoside linkage” refers to a substitution or any change from a naturally occurring internucleoside linkage (e.g., a phosphodiester internucleoside bond).

As used herein, “modified nucleobase” means any nucleobase other than adenine, cytosine, guanine, thymine, or uracil. Examples of a modified nucleobase include 5-methyl cytosine, pseudouridine, or 5-methoxyuridine. An “unmodified nucleobase” means the purine bases adenine (A) and guanine (G), and the pyrimidine bases thymine (T), cytosine (C), and uracil (U).

As used herein, a “modified nucleoside” means a nucleoside having, independently, a modified sugar moiety and/or modified nucleobase. A universal base is a modified nucleobase that can pair with any one of the five unmodified nucleobases. Modified nucleosides include abasic nucleosides, which lack a nucleobase. However, modified nucleosides do not include spacers or other groups that are incapable of linking a nucleobase.

As used herein, “linked nucleosides” are nucleosides that are connected in a contiguous sequence (i.e., no additional nucleosides are presented between those that are linked). In various embodiments, an oligonucleotide may have different segments of linked nucleosides connected through a spacer. Here, the spacer (i.e., nucleoside replacement) is not considered a nucleoside and therefore, divides up the oligonucleotide into two segments of linked nucleosides. The oligonucleotide may have a first segment of Y linked nucleosides (e.g., Y nucleosides that are connected in a contiguous sequence), followed by a spacer, and then a second segment of Z linked nucleosides. Here, the Y and Z linked nucleosides is described in either the 5′ to 3′ direction or the 3′ to 5′ direction. In various embodiments, the first segment consists of 7 or fewer linked nucleosides (e.g., Y=7 or fewer) whereas the second segment comprises 8 or more linked nucleosides (e.g., Z=8 or more).

As used herein, “modified oligonucleotide” means an oligonucleotide comprising at least one (i.e., one or more) modified internucleoside linkage, modified sugar, and/or modified nucleobase.

As used herein, “modified sugar” or “modified sugar moiety” means a modified furanosyl sugar moiety or a modified sugar moiety having other than a furanosyl moiety that can link a nucleobase to another group, such as an internucleoside linkage, conjugate group, or terminal group in an oligonucleotide.

As used herein, “monomer” means a single unit of an oligomer. Monomers include, but are not limited to, nucleosides and nucleotides, whether naturally occurring or modified.

As used herein, “motif” means the pattern of unmodified and modified nucleosides in an antisense compound.

As used herein, “natural sugar moiety” means a sugar moiety found in DNA (2′-H) or RNA (2′-OH).

As used herein, “naturally occurring internucleoside linkage” means a 3′ to 5′ phosphodiester linkage.

As used herein, “non-complementary nucleobases” refers to a pair of nucleobases that do not form hydrogen bonds with one another or otherwise support hybridization.

As used herein, “nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, non-coding RNA, small interfering ribonucleic acids (siRNA), short-hairpin RNA (shRNA), and microRNAs (miRNA).

As used herein, “nucleobase” means a heterocyclic moiety capable of base pairing with a base of another nucleic acid.

As used herein, “nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a corresponding nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, “nucleobase sequence” means the order of nucleobases independent of any sugar, linkage, and/or nucleobase modification.

As used herein, “nucleoside” refers to a nucleobase linked to a sugar. The term “nucleoside” also includes a “modified nucleoside” which has independently, a modified sugar moiety and/or modified nucleobase.

As used herein, “nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non-furanose sugar units. Nucleotide mimetic includes those structures used to replace the nucleoside and the linkage at one or more positions of an oligomeric compound such as for example peptide nucleic acids or morpholinos (morpholinos linked by a phosphorodiamidate or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

As used herein, “nucleotide” means a nucleoside having a phosphate group covalently linked to the sugar portion of the nucleoside.

As used herein, “oligomeric compound” or “oligomer” means a polymer of linked monomeric subunits which is capable of hybridizing to at least a region of a nucleic acid molecule.

As used herein, “oligonucleotide” means a polymer of one or more segments of linked nucleosides each of which can be modified or unmodified, independent one from another.

As used herein, “hotspot region” is a range of nucleobases on a target nucleic acid amenable to oligomeric compound-mediated modulation of the splicing of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding between complementary nucleobases.

As used herein, “increasing the amount of activity” refers to more transcriptional expression, more accurate splicing resulting in full length mature mRNA and/or protein expression, and/or more activity relative to the transcriptional expression or activity in an untreated or control sample.

### Antisense Therapeutics

Antisense therapeutics are a class of nucleic acid-based compounds that can be used to modulate a transcript, such as mRNA. In various embodiments, antisense therapeutics comprise one or more spacers and can be used to modulate a transcript that is transcribed from a gene, such as a UNC13A pre-mRNA.

Antisense therapeutics may be single- or double-stranded deoxyribonucleic acid (DNA)-based, ribonucleic acid (RNA)-based, or DNA/RNA chemical analogue compounds. In general, antisense therapeutics are designed to include a sequence that is complementary or nearly complementary to an mRNA or pre-mRNA sequence transcribed from a given gene in order to promote binding between the antisense therapeutic and the pre-mRNA or mRNA. In certain embodiments, antisense therapeutics act by binding to an mRNA or pre-mRNA, thereby inhibiting protein translation, altering pre-mRNA splicing into mature mRNA (e.g., by preventing appropriate proteins such as splicing activator proteins from binding), and/or causing destruction of mRNA. In certain embodiments, the antisense therapeutic sequence is complementary to a portion of a targeted gene's or mRNA's sense sequence. In certain embodiments, antisense therapeutics described herein are oligonucleotide-based compounds that include an oligonucleotide sequence complementary to a pre-mRNA sense, or a portion thereof, and one or more spacers. In certain embodiments, antisense therapeutics described herein can also be nucleotide chemical analog-based compounds.

In certain embodiments, an oligonucleotide, such as disclosed herein, may be an oligonucleotide sequence of 5 to 100 oligonucleotide units in length, for example, 10 to 60 oligonucleotide units in length, for example, 12 to 50 oligonucleotide units in length, 14 to 40 oligonucleotide units in length, 10 to 30 oligonucleotide units in length, for example, 14 to 30 oligonucleotide units in length, for example, 14 to 25 or 15 to 22 oligonucleotide units in length, or 18, 19, 20, 21, 22, 23, 24, 25, 26, or 27 oligonucleotide units in length. As used herein, an “oligonucleotide unit” refers to either a nucleoside (e.g., a nucleoside which includes a sugar and/or a nucleobase) or a nucleoside-replacement group (e.g., a spacer) of the oligonucleotide.

In particular embodiments, the oligonucleotides are 25 oligonucleotide units in length. In particular embodiments, the oligonucleotides are 23 oligonucleotide units in length. In particular embodiments, the oligonucleotides are 21 oligonucleotide units in length. In particular embodiments, the oligonucleotides are 19 oligonucleotide units in length. In various embodiments, the oligonucleotide is at least 18, at least 19, at least 20, at least 21, at least 22, at least 23, at least 24, at least 25, at least 26, or at least 27 oligonucleotide units in length. In various embodiments, the oligonucleotide is at least 18 oligonucleotide units in length. In various embodiments, the oligonucleotide is at least 19 oligonucleotide units in length. In various embodiments, the oligonucleotide is at least 20 oligonucleotide units in length. In various embodiments, the oligonucleotide is at least 21 oligonucleotide units in length. In various embodiments, the oligonucleotide is at least 22 oligonucleotide units in length. In various embodiments, the oligonucleotide is at least 23 oligonucleotide units in length. In various embodiments, the oligonucleotide is at least 24 oligonucleotide units in length. In various embodiments, the oligonucleotide is at least 25 oligonucleotide units in length. In various embodiments, the oligonucleotide is at least 26 oligonucleotide units in length. In various embodiments, the oligonucleotide is at least 27 oligonucleotide units in length.

In certain embodiments, AONs may include chemically modified nucleosides (for example, 2′-O-methylated nucleosides or 2′-O-(2-methoxyethyl) nucleosides) as well as modified internucleoside linkages (for example, phosphorothioate linkages). In certain embodiments, AONs described herein include oligonucleotide sequences that are complementary to RNA sequences, such as UNC13A mRNA sequences. In certain embodiments, AONs described herein can include chemically modified nucleosides and modified internucleoside linkages (for example, phosphorothioate linkages). In particular embodiments, AONs described herein include one or more spacers.

In various embodiments, the oligonucleotides comprise one or more spacers. In particular embodiments, the oligonucleotides comprise one spacer. In various embodiments, the oligonucleotides comprise two spacers. For example, the oligonucleotide includes 23 oligonucleotide units with 21 nucleobases and two nucleoside replacement groups (e.g., two spacers). Further embodiments of oligonucleotides with one spacer and oligonucleotides with two spacers are described herein. In various embodiments, the oligonucleotides comprise three spacers.

In some embodiments, an antisense oligonucleotide can be, but is not limited to, inhibitors of a gene transcript (for example, shRNAs, siRNAs, PNAs, LNAs, 2′-O-methyl (2′OMe) antisense oligonucleotide (AON), 2′-O-(2-methoxyethyl) (MOE) AON, or morpholino oligomers (e.g., phosphorodiamidate morpholino (PMO))), or compositions that include such compounds. In some embodiments an oligonucleotide is an antisense oligonucleotide (AON) comprising 2′OMe (e.g., a AON comprising one or more 2′OMe modified sugar), MOE (e.g., a AON comprising one or more MOE modified sugar), peptide nucleic acids (e.g., a AON comprising one or more N-(2-aminoethyl)-glycine units linked by amide bonds or carbonyl methylene linkage as repeating units in place of a sugar-phosphate backbone), locked nucleic acids (e.g., a AON comprising one or more locked ribose, and can be a mixture of 2′-deoxy nucleotides or 2′OMe nucleotides), c-ET (e.g., a AON comprising one or more cET sugar), constrained methoxyethyl (cMOE) (e.g., a AON comprising one or more cMOE sugar), morpholino oligomer (e.g., a AON comprising a backbone comprising one or more PMO), deoxy-2′-fluoro nucleoside (e.g., a AON comprising one or more 2′-fluoro-β-D-arabinonucleoside), tricyclo-DNAs (tcDNA) (e.g., a AON comprising one or more tcDNA modified sugar), 2′-0,4′-C-Ethylene-bridged nucleic acid (ENA) (e.g., a AON comprising one or more ENA modified sugar), or hexitol nucleic acids (HNA) (e.g., a AON comprising one or more HNA modified sugar). In some embodiments, a AON comprises one or more internucleoside linkage independently selected from a phosphorothioate linkage, phosphodiester linkage, phosphotriester linkage, methylphosphonate linkage, phosphoramidate linkage, a phosphoramidothioate linkage, a thiophosphorodiamidate linkage, phosphorodiamidate morpholino (PMO) (morpholino) linkage, PNA linkage, or any combination of phosphorothioate linkage, phosphodiester linkage, a phosphotriester linkage, methylphosphonate linkage, phosphoramidate linkage, a phosphoramidothioate linkage, thiophosphorodiamidate linkage, phosphorodiamidate morpholino (PMO) (morpholino) linkage, and PNA linkage. In some embodiments, a UNC13A AON comprises one or more phosphorothioate linkage, phosphodiester linkage, or a combination of phosphorothioate and phosphodiester linkages.

Peptide nucleic acids (PNAs) are short, artificially synthesized polymers with a structure that mimics DNA or RNA. PNAs include a backbone composed of repeating N-(2-aminoethyl)-glycine units linked by peptide bonds. In certain embodiments, PNAs described herein can be used as antisense therapeutics that bind to RNA sequences with high specificity and increase, restore, and/or stabilize levels (e.g., full length UNC13A mRNA or protein levels) and/or activity (e.g., biological activity, for example, UNC13A activity).

Locked nucleic acids (LNAs) are oligonucleotide sequences that include one or more modified RNA nucleotides in which the ribose moiety is modified with an extra bridge connecting the 2′ oxygen and 4′ carbon. LNAs are believed to have higher Tm's than analogous oligonucleotide sequences. In certain embodiments, LNAs described herein can be used as antisense therapeutics that bind to RNA sequences with high specificity. For example, LNAs can bind to UNC13A pre-mRNA and prevent mis-splicing of UNC13A pre-mRNA, and increase, restore, and/or stabilize UNC13A levels (e.g., UNC13A mRNA or protein levels) and/or activity (e.g., biological activity, for example, UNC13A activity).

Morpholino oligomers are oligonucleotide compounds that include DNA bases attached to a backbone of methylenemorpholine rings linked through phosphorodiamidate groups. In certain embodiments, morpholino oligomers of the present invention can be designed to bind to specific pre-mRNA sequence of interest. For example, morpholino oligomers bind to UNC13A pre-mRNA thereby preventing mis-splicing of the pre-mRNA, and increase, restore, and/or stabilize UNC13A levels (e.g., UNC13A mRNA or protein levels) and/or activity (e.g., biological activity, for example, UNC13A activity). In certain embodiments, UNC13A morpholino oligomers described herein can be used as antisense therapeutics that bind to UNC13A pre-mRNA sequences with high specificity and prevent mis-splicing of UNC13A pre-mRNA, and increase, restore, and/or stabilize UNC13A levels (e.g., UNC13A mRNA or protein levels) and/or activity (e.g., biological activity, for example, UNC13A activity). In certain embodiments, UNC13A morpholino oligomers described herein can also be used to bind UNC13A pre-mRNA sequences, altering UNC13A pre-mRNA splicing and UNC13A gene expression, and increase, restore, and/or stabilize UNC13A levels (e.g., UNC13A mRNA or protein levels) and/or activity (e.g., biological activity, for example, UNC13A activity).

### UNC13A Oligonucleotides Complementary to UNC13A Transcript

In some embodiments, a UNC13A AON includes a sequence that is at least % complementary to a sequence that shares at least 90% (e.g., 90%, 91%, 92%, 93%, 94%, 95%, 96%, 97%, 98%, 99%, or 100%) identity to a region of a UNC13A transcript (e.g., SEQ ID NO: 5057-5065 or SEQ ID NOs: 5206-5208). In some embodiments, a UNC13A AON includes a sequence that is between 90-95% complementary to a sequence that shares at least 90% (e.g., 90%, 91%, 92%, 93%, 94%, 95%, 96%, 97%, 98%, 99%, or 100%) identity to a region of a UNC13A transcript (e.g., SEQ ID NO: 5057-5065 or SEQ ID NOs: 5206-5208). In particular embodiments, a UNC13A AON includes a sequence that is at least 85% complementary to a sequence that shares at least 90% (e.g., 90%, 91%, 92%, 93%, 94%, 95%, 96%, 97%, 98%, 99%, or 100%) identity to a region of a UNC13A transcript (e.g., SEQ ID NO: 5057-5065 or SEQ ID NOs: 5206-5208). In particular embodiments, a UNC13A AON includes a sequence that is between 84% to 88% complementary to a sequence that shares at least 90% (e.g., 90%, 91%, 92%, 93%, 94%, 95%, 96%, 97%, 98%, 99%, or 100%) identity to a region of a UNC13A transcript (e.g., SEQ ID NO: 5057-5065 or SEQ ID NOs: 5206-5208). In particular embodiments, a UNC13A AON includes a sequence that is between 89% to 92% complementary to a sequence that shares at least 90% (e.g., 90%, 91%, 92%, 93%, 94%, 95%, 96%, 97%, 98%, 99%, or 100%) identity to a region of a mis-spliced UNC13A transcript (e.g., SEQ ID NO: 5057-5065). In particular embodiments, a UNC13A AON includes a sequence that is between 94% to 96% complementary to a sequence that shares at least 90% (e.g., 90%, 91%, 92%, 93%, 94%, 95%, 96%, 97%, 98%, 99%, or 100%) identity to a region of a UNC13A transcript (e.g., SEQ ID NO: 5057-5065 or SEQ ID NOs: 5206-5208).

In various embodiments, a UNC13A AON comprises a sequence that shares at least 85% identity with an equal length portion of any one of SEQ ID NOs: 1-1264, SEQ ID NOs: 2529-3792, SEQ ID NOs: 5066-5166, SEQ ID NOs: 5168-5202, SEQ ID NOs: 5209-5221, or SEQ ID NOs: 5235-5292. In various embodiments, a UNC13A AON comprises a sequence that shares at least 90% identity with an equal length portion of any one of SEQ ID NOs: 1-1264, SEQ ID NOs: 2529-3792, SEQ ID NOs: 5066-5166, SEQ ID NOs: 5168-5202, SEQ ID NOs: 5209-5221, or SEQ ID NOs: 5235-5292.

In some embodiments, the UNC13A AON comprises a spacer and has a segment having at most 7 linked nucleosides. In some embodiments, the UNC13A AON comprises a spacer and has a segment having at most 6, 5, 4, 3, or 2 linked nucleosides.

UNC13A AON binding specificity can be assessed via measurement of parameters such as dissociation constant, melting temperature, or other criteria such as changes in protein or RNA expression levels or other assays that measure UNC13A activity or expression.

In some embodiments, a UNC13A AON can include a non-duplexed oligonucleotide. In some embodiments, a UNC13A AON can include a duplex of two oligonucleotides where the first oligonucleotide includes a nucleobase sequence that is completely or almost completely complementary to a UNC13A pre-mRNA sequence and the second oligonucleotide includes a nucleobase sequence that is complementary to the nucleobase sequence of the first oligonucleotide.

In some embodiments, a UNC13A AON can target UNC13A pre-mRNAs produced from UNC13A genes of one or more species. For example, a UNC13A AON can target a UNC13A pre-mRNA of a mammalian UNC13A gene, for example, a human (i.e., Homo sapiens) UNC13A gene. In particular embodiments, the UNC13A AON targets a human UNC13A pre-mRNA. In some embodiments, the UNC13A AON includes a nucleobase sequence that is complementary to a nucleobase sequence of a UNC13A gene or a UNC13A pre-mRNA or a portion thereof.

UNC13A AONs described herein include antisense oligonucleotides comprising the oligonucleotide sequences listed in Table 1 below:

TABLE 1
Example UNC13A AON Sequences.

| SEQ | | SEQ | |
|---|---|---|---|
| ID | AON Sequence* | ID | Target Sequence |
| NO: | (5′ → 3′) | NO: | (5′ → 3′) |
| 1 | GGAGGGGCCGAGCAATGACCCTCAC | 1265 | GTGAGGGTCATTGCTCGGCCCCTCC |
| 2 | GGGAGGGGCCGAGCAATGACCCTCA | 1266 | TGAGGGTCATTGCTCGGCCCCTCCC |
| 3 | TGGGAGGGGCCGAGCAATGACCCTC | 1267 | GAGGGTCATTGCTCGGCCCCTCCCA |
| 4 | ATGGGAGGGGCCGAGCAATGACCCT | 1268 | AGGGTCATTGCTCGGCCCCTCCCAT |
| 5 | CATGGGAGGGGCCGAGCAATGACCC | 1269 | GGGTCATTGCTCGGCCCCTCCCATG |
| 6 | GCATGGGAGGGGCCGAGCAATGACC | 1270 | GGTCATTGCTCGGCCCCTCCCATGC |
| 7 | GGCATGGGAGGGGCCGAGCAATGAC | 1271 | GTCATTGCTCGGCCCCTCCCATGCC |
| 8 | TGGCATGGGAGGGGCCGAGCAATGA | 1272 | TCATTGCTCGGCCCCTCCCATGCCA |
| 9 | GTGGCATGGGAGGGGCCGAGCAATG | 1273 | CATTGCTCGGCCCCTCCCATGCCAC |
| 10 | AGTGGCATGGGAGGGGCCGAGCAAT | 1274 | ATTGCTCGGCCCCTCCCATGCCACT |
| 11 | AAGTGGCATGGGAGGGGCCGAGCAA | 1275 | TTGCTCGGCCCCTCCCATGCCACTT |
| 12 | GAAGTGGCATGGGAGGGGCCGAGCA | 1276 | TGCTCGGCCCCTCCCATGCCACTTC |
| 13 | GGAAGTGGCATGGGAGGGGCCGAGC | 1277 | GCTCGGCCCCTCCCATGCCACTTCC |
| 14 | TGGAAGTGGCATGGGAGGGGCCGAG | 1278 | CTCGGCCCCTCCCATGCCACTTCCA |
| 15 | GTGGAAGTGGCATGGGAGGGGCCGA | 1279 | TCGGCCCCTCCCATGCCACTTCCAC |
| 16 | AGTGGAAGTGGCATGGGAGGGGCCG | 1280 | CGGCCCCTCCCATGCCACTTCCACT |
| 17 | GAGTGGAAGTGGCATGGGAGGGGCC | 1281 | GGCCCCTCCCATGCCACTTCCACTC |
| 18 | TGAGTGGAAGTGGCATGGGAGGGGC | 1282 | GCCCCTCCCATGCCACTTCCACTCA |
| 19 | GTGAGTGGAAGTGGCATGGGAGGGG | 1283 | CCCCTCCCATGCCACTTCCACTCAC |
| 20 | GGTGAGTGGAAGTGGCATGGGAGGG | 1284 | CCCTCCCATGCCACTTCCACTCACC |
| 21 | TGGTGAGTGGAAGTGGCATGGGAGG | 1285 | CCTCCCATGCCACTTCCACTCACCA |
| 22 | ATGGTGAGTGGAAGTGGCATGGGAG | 1286 | CTCCCATGCCACTTCCACTCACCAT |
| 23 | AATGGTGAGTGGAAGTGGCATGGGA | 1287 | TCCCATGCCACTTCCACTCACCATT |
| 24 | GAATGGTGAGTGGAAGTGGCATGGG | 1288 | CCCATGCCACTTCCACTCACCATTC |
| 25 | GGAATGGTGAGTGGAAGTGGCATGG | 1289 | CCATGCCACTTCCACTCACCATTCC |
| 26 | AGGAATGGTGAGTGGAAGTGGCATG | 1290 | CATGCCACTTCCACTCACCATTCCT |
| 27 | CAGGAATGGTGAGTGGAAGTGGCAT | 1291 | ATGCCACTTCCACTCACCATTCCTG |
| 28 | GCAGGAATGGTGAGTGGAAGTGGCA | 1292 | TGCCACTTCCACTCACCATTCCTGC |
| 29 | GGCAGGAATGGTGAGTGGAAGTGGC | 1293 | GCCACTTCCACTCACCATTCCTGCC |
| 30 | AGGCAGGAATGGTGAGTGGAAGTGG | 1294 | CCACTTCCACTCACCATTCCTGCCT |
| 31 | CAGGCAGGAATGGTGAGTGGAAGTG | 1295 | CACTTCCACTCACCATTCCTGCCTG |
| 32 | GCAGGCAGGAATGGTGAGTGGAAGT | 1296 | ACTTCCACTCACCATTCCTGCCTGC |
| 33 | GGCAGGCAGGAATGGTGAGTGGAAG | 1297 | CTTCCACTCACCATTCCTGCCTGCC |
| 34 | GGGCAGGCAGGAATGGTGAGTGGAA | 1298 | TTCCACTCACCATTCCTGCCTGCCC |
| 35 | TGGGCAGGCAGGAATGGTGAGTGGA | 1299 | TCCACTCACCATTCCTGCCTGCCCA |
| 36 | CTGGGCAGGCAGGAATGGTGAGTGG | 1300 | CCACTCACCATTCCTGCCTGCCCAG |
| 37 | GCTGGGCAGGCAGGAATGGTGAGTG | 1301 | CACTCACCATTCCTGCCTGCCCAGC |
| 38 | AGCTGGGCAGGCAGGAATGGTGAGT | 1302 | ACTCACCATTCCTGCCTGCCCAGCT |
| 39 | GAGCTGGGCAGGCAGGAATGGTGAG | 1303 | CTCACCATTCCTGCCTGCCCAGCTC |
| 40 | AGAGCTGGGCAGGCAGGAATGGTGA | 1304 | TCACCATTCCTGCCTGCCCAGCTCT |
| 41 | AAGAGCTGGGCAGGCAGGAATGGTG | 1305 | CACCATTCCTGCCTGCCCAGCTCTT |
| 42 | GAAGAGCTGGGCAGGCAGGAATGGT | 1306 | ACCATTCCTGCCTGCCCAGCTCTTC |
| 43 | GGAAGAGCTGGGCAGGCAGGAATGG | 1307 | CCATTCCTGCCTGCCCAGCTCTTCC |
| 44 | AGGAAGAGCTGGGCAGGCAGGAATG | 1308 | CATTCCTGCCTGCCCAGCTCTTCCT |
| 45 | GAGGAAGAGCTGGGCAGGCAGGAAT | 1309 | ATTCCTGCCTGCCCAGCTCTTCCTC |
| 46 | AGAGGAAGAGCTGGGCAGGCAGGAA | 1310 | TTCCTGCCTGCCCAGCTCTTCCTCT |
| 47 | AAGAGGAAGAGCTGGGCAGGCAGGA | 1311 | TCCTGCCTGCCCAGCTCTTCCTCTT |
| 48 | AAAGAGGAAGAGCTGGGCAGGCAGG | 1312 | CCTGCCTGCCCAGCTCTTCCTCTTT |
| 49 | GAAAGAGGAAGAGCTGGGCAGGCAG | 1313 | CTGCCTGCCCAGCTCTTCCTCTTTC |
| 50 | AGAAAGAGGAAGAGCTGGGCAGGCA | 1314 | TGCCTGCCCAGCTCTTCCTCTTTCT |
| 51 | CAGAAAGAGGAAGAGCTGGGCAGGC | 1315 | GCCTGCCCAGCTCTTCCTCTTTCTG |
| 52 | CCAGAAAGAGGAAGAGCTGGGCAGG | 1316 | CCTGCCCAGCTCTTCCTCTTTCTGG |
| 53 | GCCAGAAAGAGGAAGAGCTGGGCAG | 1317 | CTGCCCAGCTCTTCCTCTTTCTGGC |
| 54 | GGCCAGAAAGAGGAAGAGCTGGGCA | 1318 | TGCCCAGCTCTTCCTCTTTCTGGCC |
| 55 | TGGCCAGAAAGAGGAAGAGCTGGGC | 1319 | GCCCAGCTCTTCCTCTTTCTGGCCA |
| 56 | GTGGCCAGAAAGAGGAAGAGCTGGG | 1320 | CCCAGCTCTTCCTCTTTCTGGCCAC |
| 57 | TGTGGCCAGAAAGAGGAAGAGCTGG | 1321 | CCAGCTCTTCCTCTTTCTGGCCACA |
| 58 | GTGTGGCCAGAAAGAGGAAGAGCTG | 1322 | CAGCTCTTCCTCTTTCTGGCCACAC |
| 59 | GGTGTGGCCAGAAAGAGGAAGAGCT | 1323 | AGCTCTTCCTCTTTCTGGCCACACC |
| 60 | TGGTGTGGCCAGAAAGAGGAAGAGC | 1324 | GCTCTTCCTCTTTCTGGCCACACCA |
| 61 | ATGGTGTGGCCAGAAAGAGGAAGAG | 1325 | CTCTTCCTCTTTCTGGCCACACCAT |
| 62 | GATGGTGTGGCCAGAAAGAGGAAGA | 1326 | TCTTCCTCTTTCTGGCCACACCATC |
| 63 | GGATGGTGTGGCCAGAAAGAGGAAG | 1327 | CTTCCTCTTTCTGGCCACACCATCC |
| 64 | TGGATGGTGTGGCCAGAAAGAGGAA | 1328 | TTCCTCTTTCTGGCCACACCATCCA |
| 65 | GTGGATGGTGTGGCCAGAAAGAGGA | 1329 | TCCTCTTTCTGGCCACACCATCCAC |
| 66 | TGTGGATGGTGTGGCCAGAAAGAGG | 1330 | CCTCTTTCTGGCCACACCATCCACA |
| 67 | GTGTGGATGGTGTGGCCAGAAAGAG | 1331 | CTCTTTCTGGCCACACCATCCACAC |
| 68 | AGTGTGGATGGTGTGGCCAGAAAGA | 1332 | TCTTTCTGGCCACACCATCCACACT |
| 69 | GAGTGTGGATGGTGTGGCCAGAAAG | 1333 | CTTTCTGGCCACACCATCCACACTC |
| 70 | AGAGTGTGGATGGTGTGGCCAGAAA | 1334 | TTTCTGGCCACACCATCCACACTCT |
| 71 | GAGAGTGTGGATGGTGTGGCCAGAA | 1335 | TTCTGGCCACACCATCCACACTCTC |
| 72 | GGAGAGTGTGGATGGTGTGGCCAGA | 1336 | TCTGGCCACACCATCCACACTCTCC |
| 73 | AGGAGAGTGTGGATGGTGTGGCCAG | 1337 | CTGGCCACACCATCCACACTCTCCT |
| 74 | CAGGAGAGTGTGGATGGTGTGGCCA | 1338 | TGGCCACACCATCCACACTCTCCTG |
| 75 | CCAGGAGAGTGTGGATGGTGTGGCC | 1339 | GGCCACACCATCCACACTCTCCTGG |
| 76 | GCCAGGAGAGTGTGGATGGTGTGGC | 1340 | GCCACACCATCCACACTCTCCTGGC |
| 77 | GGCCAGGAGAGTGTGGATGGTGTGG | 1341 | CCACACCATCCACACTCTCCTGGCC |
| 78 | GGGCCAGGAGAGTGTGGATGGTGTG | 1342 | CACACCATCCACACTCTCCTGGCCC |
| 79 | AGGGCCAGGAGAGTGTGGATGGTGT | 1343 | ACACCATCCACACTCTCCTGGCCCT |
| 80 | GAGGGCCAGGAGAGTGTGGATGGTG | 1344 | CACCATCCACACTCTCCTGGCCCTC |
| 81 | AGAGGGCCAGGAGAGTGTGGATGGT | 1345 | ACCATCCACACTCTCCTGGCCCTCT |
| 82 | CAGAGGGCCAGGAGAGTGTGGATGG | 1346 | CCATCCACACTCTCCTGGCCCTCTG |
| 83 | TCAGAGGGCCAGGAGAGTGTGGATG | 1347 | CATCCACACTCTCCTGGCCCTCTGA |
| 84 | CTCAGAGGGCCAGGAGAGTGTGGAT | 1348 | ATCCACACTCTCCTGGCCCTCTGAG |
| 85 | TCTCAGAGGGCCAGGAGAGTGTGGA | 1349 | TCCACACTCTCCTGGCCCTCTGAGA |
| 86 | GTCTCAGAGGGCCAGGAGAGTGTGG | 1350 | CCACACTCTCCTGGCCCTCTGAGAC |
| 87 | AGTCTCAGAGGGCCAGGAGAGTGTG | 1351 | CACACTCTCCTGGCCCTCTGAGACT |
| 88 | CAGTCTCAGAGGGCCAGGAGAGTGT | 1352 | ACACTCTCCTGGCCCTCTGAGACTG |
| 89 | GCAGTCTCAGAGGGCCAGGAGAGTG | 1353 | CACTCTCCTGGCCCTCTGAGACTGC |
| 90 | GGCAGTCTCAGAGGGCCAGGAGAGT | 1354 | ACTCTCCTGGCCCTCTGAGACTGCC |
| 91 | GGGCAGTCTCAGAGGGCCAGGAGAG | 1355 | CTCTCCTGGCCCTCTGAGACTGCCC |
| 92 | CGGGCAGTCTCAGAGGGCCAGGAGA | 1356 | TCTCCTGGCCCTCTGAGACTGCCCG |
| 93 | GCGGGCAGTCTCAGAGGGCCAGGAG | 1357 | CTCCTGGCCCTCTGAGACTGCCCGC |
| 94 | GGCGGGCAGTCTCAGAGGGCCAGGA | 1358 | TCCTGGCCCTCTGAGACTGCCCGCC |
| 95 | TGGCGGGCAGTCTCAGAGGGCCAGG | 1359 | CCTGGCCCTCTGAGACTGCCCGCCA |
| 96 | ATGGCGGGCAGTCTCAGAGGGCCAG | 1360 | CTGGCCCTCTGAGACTGCCCGCCAT |
| 97 | CATGGCGGGCAGTCTCAGAGGGCCA | 1361 | TGGCCCTCTGAGACTGCCCGCCATG |
| 98 | GCATGGCGGGCAGTCTCAGAGGGCC | 1362 | GGCCCTCTGAGACTGCCCGCCATGC |
| 99 | GGCATGGCGGGCAGTCTCAGAGGGC | 1363 | GCCCTCTGAGACTGCCCGCCATGCC |
| 100 | TGGCATGGCGGGCAGTCTCAGAGGG | 1364 | CCCTCTGAGACTGCCCGCCATGCCA |
| 101 | ATGGCATGGCGGGCAGTCTCAGAGG | 1365 | CCTCTGAGACTGCCCGCCATGCCAT |
| 102 | AATGGCATGGCGGGCAGTCTCAGAG | 1366 | CTCTGAGACTGCCCGCCATGCCATT |
| 103 | GAATGGCATGGCGGGCAGTCTCAGA | 1367 | TCTGAGACTGCCCGCCATGCCATTC |
| 104 | GGAATGGCATGGCGGGCAGTCTCAG | 1368 | CTGAGACTGCCCGCCATGCCATTCC |
| 105 | GGGAATGGCATGGCGGGCAGTCTCA | 1369 | TGAGACTGCCCGCCATGCCATTCCC |
| 106 | AGGGAATGGCATGGCGGGCAGTCTC | 1370 | GAGACTGCCCGCCATGCCATTCCCT |
| 107 | AAGGGAATGGCATGGGGGCAGTCT | 1371 | AGACTGCCCGCCATGCCATTCCCTT |
| 108 | AAAGGGAATGGCATGGCGGGCAGTC | 1372 | GACTGCCCGCCATGCCATTCCCTTT |
| 109 | TAAAGGGAATGGCATGGCGGGCAGT | 1373 | ACTGCCCGCCATGCCATTCCCTTTA |
| 110 | GTAAAGGGAATGGCATGGCGGGCAG | 1374 | CTGCCCGCCATGCCATTCCCTTTAC |
| 111 | GGTAAAGGGAATGGCATGGCGGGCA | 1375 | TGCCCGCCATGCCATTCCCTTTACC |
| 112 | AGGTAAAGGGAATGGCATGGCGGGC | 1376 | GCCCGCCATGCCATTCCCTTTACCT |
| 113 | CAGGTAAAGGGAATGGCATGGCGGG | 1377 | CCCGCCATGCCATTCCCTTTACCTG |
| 114 | CCAGGTAAAGGGAATGGCATGGCGG | 1378 | CCGCCATGCCATTCCCTTTACCTGG |
| 115 | TCCAGGTAAAGGGAATGGCATGGCG | 1379 | CGCCATGCCATTCCCTTTACCTGGA |
| 116 | TTCCAGGTAAAGGGAATGGCATGGC | 1380 | GCCATGCCATTCCCTTTACCTGGAA |
| 117 | TTTCCAGGTAAAGGGAATGGCATGG | 1381 | CCATGCCATTCCCTTTACCTGGAAA |
| 118 | TTTTCCAGGTAAAGGGAATGGCATG | 1382 | CATGCCATTCCCTTTACCTGGAAAA |
| 119 | GTTTTCCAGGTAAAGGGAATGGCAT | 1383 | ATGCCATTCCCTTTACCTGGAAAAC |
| 120 | AGTTTTCCAGGTAAAGGGAATGGCA | 1384 | TGCCATTCCCTTTACCTGGAAAACT |
| 121 | GAGTTTTCCAGGTAAAGGGAATGGC | 1385 | GCCATTCCCTTTACCTGGAAAACTC |
| 122 | GGAGTTTTCCAGGTAAAGGGAATGG | 1386 | CCATTCCCTTTACCTGGAAAACTCC |
| 123 | AGGAGTTTTCCAGGTAAAGGGAATG | 1387 | CATTCCCTTTACCTGGAAAACTCCT |
| 124 | GAGGAGTTTTCCAGGTAAAGGGAAT | 1388 | ATTCCCTTTACCTGGAAAACTCCTC |
| 125 | GGAGGAGTTTTCCAGGTAAAGGGAA | 1389 | TTCCCTTTACCTGGAAAACTCCTCC |
| 126 | GGGAGGAGTTTTCCAGGTAAAGGGA | 1390 | TCCCTTTACCTGGAAAACTCCTCCC |
| 127 | AGGGAGGAGTTTTCCAGGTAAAGGG | 1391 | CCCTTTACCTGGAAAACTCCTCCCT |
| 128 | TAGGGAGGAGTTTTCCAGGTAAAGG | 1392 | CCTTTACCTGGAAAACTCCTCCCTA |
| 129 | ATAGGGAGGAGTTTTCCAGGTAAAG | 1393 | CTTTACCTGGAAAACTCCTCCCTAT |
| 130 | GATAGGGAGGAGTTTTCCAGGTAAA | 1394 | TTTACCTGGAAAACTCCTCCCTATC |
| 131 | GGATAGGGAGGAGTTTTCCAGGTAA | 1395 | TTACCTGGAAAACTCCTCCCTATCC |
| 132 | TGGATAGGGAGGAGTTTTCCAGGTA | 1396 | TACCTGGAAAACTCCTCCCTATCCA |
| 133 | ATGGATAGGGAGGAGTTTTCCAGGT | 1397 | ACCTGGAAAACTCCTCCCTATCCAT |
| 134 | GATGGATAGGGAGGAGTTTTCCAGG | 1398 | CCTGGAAAACTCCTCCCTATCCATC |
| 135 | TGATGGATAGGGAGGAGTTTTCCAG | 1399 | CTGGAAAACTCCTCCCTATCCATCA |
| 136 | TTGATGGATAGGGAGGAGTTTTCCA | 1400 | TGGAAAACTCCTCCCTATCCATCAA |
| 137 | TTTGATGGATAGGGAGGAGTTTTCC | 1401 | GGAAAACTCCTCCCTATCCATCAAA |
| 138 | CTTTGATGGATAGGGAGGAGTTTTC | 1402 | GAAAACTCCTCCCTATCCATCAAAG |
| 139 | ACTTTGATGGATAGGGAGGAGTTTT | 1403 | AAAACTCCTCCCTATCCATCAAAGT |
| 140 | GACTTTGATGGATAGGGAGGAGTTT | 1404 | AAACTCCTCCCTATCCATCAAAGTC |
| 141 | GGACTTTGATGGATAGGGAGGAGTT | 1405 | AACTCCTCCCTATCCATCAAAGTCC |
| 142 | TGGACTTTGATGGATAGGGAGGAGT | 1406 | ACTCCTCCCTATCCATCAAAGTCCA |
| 143 | CTGGACTTTGATGGATAGGGAGGAG | 1407 | CTCCTCCCTATCCATCAAAGTCCAG |
| 144 | TCTGGACTTTGATGGATAGGGAGGA | 1408 | TCCTCCCTATCCATCAAAGTCCAGA |
| 145 | ATCTGGACTTTGATGGATAGGGAGG | 1409 | CCTCCCTATCCATCAAAGTCCAGAT |
| 146 | AATCTGGACTTTGATGGATAGGGAG | 1410 | CTCCCTATCCATCAAAGTCCAGATT |
| 147 | GAATCTGGACTTTGATGGATAGGGA | 1411 | TCCCTATCCATCAAAGTCCAGATTC |
| 148 | TGAATCTGGACTTTGATGGATAGGG | 1412 | CCCTATCCATCAAAGTCCAGATTCA |
| 149 | CTGAATCTGGACTTTGATGGATAGG | 1413 | CCTATCCATCAAAGTCCAGATTCAG |
| 150 | CCTGAATCTGGACTTTGATGGATAG | 1414 | CTATCCATCAAAGTCCAGATTCAGG |
| 151 | CCCTGAATCTGGACTTTGATGGATA | 1415 | TATCCATCAAAGTCCAGATTCAGGG |
| 152 | ACCCTGAATCTGGACTTTGATGGAT | 1416 | ATCCATCAAAGTCCAGATTCAGGGT |
| 153 | GACCCTGAATCTGGACTTTGATGGA | 1417 | TCCATCAAAGTCCAGATTCAGGGTC |
| 154 | TGACCCTGAATCTGGACTTTGATGG | 1418 | CCATCAAAGTCCAGATTCAGGGTCA |
| 155 | GTGACCCTGAATCTGGACTTTGATG | 1419 | CATCAAAGTCCAGATTCAGGGTCAC |
| 156 | GGTGACCCTGAATCTGGACTTTGAT | 1420 | ATCAAAGTCCAGATTCAGGGTCACC |
| 157 | AGGTGACCCTGAATCTGGACTTTGA | 1421 | TCAAAGTCCAGATTCAGGGTCACCT |
| 158 | GAGGTGACCCTGAATCTGGACTTTG | 1422 | CAAAGTCCAGATTCAGGGTCACCTC |
| 159 | GGAGGTGACCCTGAATCTGGACTTT | 1423 | AAAGTCCAGATTCAGGGTCACCTCC |
| 160 | AGGAGGTGACCCTGAATCTGGACTT | 1424 | AAGTCCAGATTCAGGGTCACCTCCT |
| 161 | GAGGAGGTGACCCTGAATCTGGACT | 1425 | AGTCCAGATTCAGGGTCACCTCCTC |
| 162 | AGAGGAGGTGACCCTGAATCTGGAC | 1426 | GTCCAGATTCAGGGTCACCTCCTCT |
| 163 | CAGAGGAGGTGACCCTGAATCTGGA | 1427 | TCCAGATTCAGGGTCACCTCCTCTG |
| 164 | CCAGAGGAGGTGACCCTGAATCTGG | 1428 | CCAGATTCAGGGTCACCTCCTCTGG |
| 165 | CCCAGAGGAGGTGACCCTGAATCTG | 1429 | CAGATTCAGGGTCACCTCCTCTGGG |
| 166 | TCCCAGAGGAGGTGACCCTGAATCT | 1430 | AGATTCAGGGTCACCTCCTCTGGGA |
| 167 | TTCCCAGAGGAGGTGACCCTGAATC | 1431 | GATTCAGGGTCACCTCCTCTGGGAA |
| 168 | CTTCCCAGAGGAGGTGACCCTGAAT | 1432 | ATTCAGGGTCACCTCCTCTGGGAAG |
| 169 | GCTTCCCAGAGGAGGTGACCCTGAA | 1433 | TTCAGGGTCACCTCCTCTGGGAAGC |
| 170 | GGCTTCCCAGAGGAGGTGACCCTGA | 1434 | TCAGGGTCACCTCCTCTGGGAAGCC |
| 171 | GGGCTTCCCAGAGGAGGTGACCCTG | 1435 | CAGGGTCACCTCCTCTGGGAAGCCC |
| 172 | TGGGCTTCCCAGAGGAGGTGACCCT | 1436 | AGGGTCACCTCCTCTGGGAAGCCCA |
| 173 | GTGGGCTTCCCAGAGGAGGTGACCC | 1437 | GGGTCACCTCCTCTGGGAAGCCCAC |
| 174 | GGTGGGCTTCCCAGAGGAGGTGACC | 1438 | GGTCACCTCCTCTGGGAAGCCCACC |
| 175 | AGGTGGGCTTCCCAGAGGAGGTGAC | 1439 | GTCACCTCCTCTGGGAAGCCCACCT |
| 176 | AAGGTGGGCTTCCCAGAGGAGGTGA | 1440 | TCACCTCCTCTGGGAAGCCCACCTT |
| 177 | CAAGGTGGGCTTCCCAGAGGAGGTG | 1441 | CACCTCCTCTGGGAAGCCCACCTTG |
| 178 | CCAAGGTGGGCTTCCCAGAGGAGGT | 1442 | ACCTCCTCTGGGAAGCCCACCTTGG |
| 179 | GCCAAGGTGGGCTTCCCAGAGGAGG | 1443 | CCTCCTCTGGGAAGCCCACCTTGGC |
| 180 | GGCCAAGGTGGGCTTCCCAGAGGAG | 1444 | CTCCTCTGGGAAGCCCACCTTGGCC |
| 181 | AGGCCAAGGTGGGCTTCCCAGAGGA | 1445 | TCCTCTGGGAAGCCCACCTTGGCCT |
| 182 | GAGGCCAAGGTGGGCTTCCCAGAGG | 1446 | CCTCTGGGAAGCCCACCTTGGCCTC |
| 183 | GGAGGCCAAGGTGGGCTTCCCAGAG | 1447 | CTCTGGGAAGCCCACCTTGGCCTCC |
| 184 | TGGAGGCCAAGGTGGGCTTCCCAGA | 1448 | TCTGGGAAGCCCACCTTGGCCTCCA |
| 185 | CTGGAGGCCAAGGTGGGCTTCCCAG | 1449 | CTGGGAAGCCCACCTTGGCCTCCAG |
| 186 | CCTGGAGGCCAAGGTGGGCTTCCCA | 1450 | TGGGAAGCCCACCTTGGCCTCCAGG |
| 187 | ACCTGGAGGCCAAGGTGGGCTTCCC | 1451 | GGGAAGCCCACCTTGGCCTCCAGGT |
| 188 | AACCTGGAGGCCAAGGTGGGCTTCC | 1452 | GGAAGCCCACCTTGGCCTCCAGGTT |
| 189 | CAACCTGGAGGCCAAGGTGGGCTTC | 1453 | GAAGCCCACCTTGGCCTCCAGGTTG |
| 190 | TCAACCTGGAGGCCAAGGTGGGCTT | 1454 | AAGCCCACCTTGGCCTCCAGGTTGA |
| 191 | GTCAACCTGGAGGCCAAGGTGGGCT | 1455 | AGCCCACCTTGGCCTCCAGGTTGAC |
| 192 | AGTCAACCTGGAGGCCAAGGTGGGC | 1456 | GCCCACCTTGGCCTCCAGGTTGACT |
| 193 | GAGTCAACCTGGAGGCCAAGGTGGG | 1457 | CCCACCTTGGCCTCCAGGTTGACTC |
| 194 | AGAGTCAACCTGGAGGCCAAGGTGG | 1458 | CCACCTTGGCCTCCAGGTTGACTCT |
| 195 | GAGAGTCAACCTGGAGGCCAAGGTG | 1459 | CACCTTGGCCTCCAGGTTGACTCTC |
| 196 | TGAGAGTCAACCTGGAGGCCAAGGT | 1460 | ACCTTGGCCTCCAGGTTGACTCTCA |
| 197 | GTGAGAGTCAACCTGGAGGCCAAGG | 1461 | CCTTGGCCTCCAGGTTGACTCTCAC |
| 198 | AGTGAGAGTCAACCTGGAGGCCAAG | 1462 | CTTGGCCTCCAGGTTGACTCTCACT |
| 199 | TAGTGAGAGTCAACCTGGAGGCCAA | 1463 | TTGGCCTCCAGGTTGACTCTCACTA |
| 200 | GTAGTGAGAGTCAACCTGGAGGCCA | 1464 | TGGCCTCCAGGTTGACTCTCACTAC |
| 201 | AGTAGTGAGAGTCAACCTGGAGGCC | 1465 | GGCCTCCAGGTTGACTCTCACTACT |
| 202 | GAGTAGTGAGAGTCAACCTGGAGGC | 1466 | GCCTCCAGGTTGACTCTCACTACTC |
| 203 | TGAGTAGTGAGAGTCAACCTGGAGG | 1467 | CCTCCAGGTTGACTCTCACTACTCA |
| 204 | ATGAGTAGTGAGAGTCAACCTGGAG | 1468 | CTCCAGGTTGACTCTCACTACTCAT |
| 205 | GATGAGTAGTGAGAGTCAACCTGGA | 1469 | TCCAGGTTGACTCTCACTACTCATC |
| 206 | TGATGAGTAGTGAGAGTCAACCTGG | 1470 | CCAGGTTGACTCTCACTACTCATCA |
| 207 | ATGATGAGTAGTGAGAGTCAACCTG | 1471 | CAGGTTGACTCTCACTACTCATCAT |
| 208 | GATGATGAGTAGTGAGAGTCAACCT | 1472 | AGGTTGACTCTCACTACTCATCATC |
| 209 | TGATGATGAGTAGTGAGAGTCAACC | 1473 | GGTTGACTCTCACTACTCATCATCA |
| 210 | CTGATGATGAGTAGTGAGAGTCAAC | 1474 | GTTGACTCTCACTACTCATCATCAG |
| 211 | CCTGATGATGAGTAGTGAGAGTCAA | 1475 | TTGACTCTCACTACTCATCATCAGG |
| 212 | ACCTGATGATGAGTAGTGAGAGTCA | 1476 | TGACTCTCACTACTCATCATCAGGT |
| 213 | AACCTGATGATGAGTAGTGAGAGTC | 1477 | GACTCTCACTACTCATCATCAGGTT |
| 214 | GAACCTGATGATGAGTAGTGAGAGT | 1478 | ACTCTCACTACTCATCATCAGGTTC |
| 215 | AGAACCTGATGATGAGTAGTGAGAG | 1479 | CTCTCACTACTCATCATCAGGTTCT |
| 216 | AAGAACCTGATGATGAGTAGTGAGA | 1480 | TCTCACTACTCATCATCAGGTTCTT |
| 217 | GAAGAACCTGATGATGAGTAGTGAG | 1481 | CTCACTACTCATCATCAGGTTCTTC |
| 218 | GGAAGAACCTGATGATGAGTAGTGA | 1482 | TCACTACTCATCATCAGGTTCTTCC |
| 219 | AGGAAGAACCTGATGATGAGTAGTG | 1483 | CACTACTCATCATCAGGTTCTTCCT |
| 220 | AAGGAAGAACCTGATGATGAGTAGT | 1484 | ACTACTCATCATCAGGTTCTTCCTT |
| 221 | GAAGGAAGAACCTGATGATGAGTAG | 1485 | CTACTCATCATCAGGTTCTTCCTTC |
| 222 | AGAAGGAAGAACCTGATGATGAGTA | 1486 | TACTCATCATCAGGTTCTTCCTTCT |
| 223 | TAGAAGGAAGAACCTGATGATGAGT | 1487 | ACTCATCATCAGGTTCTTCCTTCTA |
| 224 | ATAGAAGGAAGAACCTGATGATGAG | 1488 | CTCATCATCAGGTTCTTCCTTCTAT |
| 225 | AATAGAAGGAAGAACCTGATGATGA | 1489 | TCATCATCAGGTTCTTCCTTCTATT |
| 226 | GAATAGAAGGAAGAACCTGATGATG | 1490 | CATCATCAGGTTCTTCCTTCTATTC |
| 227 | GGAATAGAAGGAAGAACCTGATGAT | 1491 | ATCATCAGGTTCTTCCTTCTATTCC |
| 228 | TGGAATAGAAGGAAGAACCTGATGA | 1492 | TCATCAGGTTCTTCCTTCTATTCCA |
| 229 | CTGGAATAGAAGGAAGAACCTGATG | 1493 | CATCAGGTTCTTCCTTCTATTCCAG |
| 230 | GCTGGAATAGAAGGAAGAACCTGAT | 1494 | ATCAGGTTCTTCCTTCTATTCCAGC |
| 231 | GGCTGGAATAGAAGGAAGAACCTGA | 1495 | TCAGGTTCTTCCTTCTATTCCAGCC |
| 232 | GGGCTGGAATAGAAGGAAGAACCTG | 1496 | CAGGTTCTTCCTTCTATTCCAGCCC |
| 233 | AGGGCTGGAATAGAAGGAAGAACCT | 1497 | AGGTTCTTCCTTCTATTCCAGCCCT |
| 234 | TAGGGCTGGAATAGAAGGAAGAACC | 1498 | GGTTCTTCCTTCTATTCCAGCCCTA |
| 235 | TTAGGGCTGGAATAGAAGGAAGAAC | 1499 | GTTCTTCCTTCTATTCCAGCCCTAA |
| 236 | GTTAGGGCTGGAATAGAAGGAAGAA | 1500 | TTCTTCCTTCTATTCCAGCCCTAAC |
| 237 | GGTTAGGGCTGGAATAGAAGGAAGA | 1501 | TCTTCCTTCTATTCCAGCCCTAACC |
| 238 | TGGTTAGGGCTGGAATAGAAGGAAG | 1502 | CTTCCTTCTATTCCAGCCCTAACCA |
| 239 | GTGGTTAGGGCTGGAATAGAAGGAA | 1503 | TTCCTTCTATTCCAGCCCTAACCAC |
| 240 | AGTGGTTAGGGCTGGAATAGAAGGA | 1504 | TCCTTCTATTCCAGCCCTAACCACT |
| 241 | GAGTGGTTAGGGCTGGAATAGAAGG | 1505 | CCTTCTATTCCAGCCCTAACCACTC |
| 242 | TGAGTGGTTAGGGCTGGAATAGAAG | 1506 | CTTCTATTCCAGCCCTAACCACTCA |
| 243 | CTGAGTGGTTAGGGCTGGAATAGAA | 1507 | TTCTATTCCAGCCCTAACCACTCAG |
| 244 | CCTGAGTGGTTAGGGCTGGAATAGA | 1508 | TCTATTCCAGCCCTAACCACTCAGG |
| 245 | TCCTGAGTGGTTAGGGCTGGAATAG | 1509 | CTATTCCAGCCCTAACCACTCAGGA |
| 246 | ATCCTGAGTGGTTAGGGCTGGAATA | 1510 | TATTCCAGCCCTAACCACTCAGGAT |
| 247 | AATCCTGAGTGGTTAGGGCTGGAAT | 1511 | ATTCCAGCCCTAACCACTCAGGATT |
| 248 | CAATCCTGAGTGGTTAGGGCTGGAA | 1512 | TTCCAGCCCTAACCACTCAGGATTG |
| 249 | CCAATCCTGAGTGGTTAGGGCTGGA | 1513 | TCCAGCCCTAACCACTCAGGATTGG |
| 250 | CCCAATCCTGAGTGGTTAGGGCTGG | 1514 | CCAGCCCTAACCACTCAGGATTGGG |
| 251 | GCCCAATCCTGAGTGGTTAGGGCTG | 1515 | CAGCCCTAACCACTCAGGATTGGGC |
| 252 | GGCCCAATCCTGAGTGGTTAGGGCT | 1516 | AGCCCTAACCACTCAGGATTGGGCC |
| 253 | CGGCCCAATCCTGAGTGGTTAGGGC | 1517 | GCCCTAACCACTCAGGATTGGGCCG |
| 254 | ACGGCCCAATCCTGAGTGGTTAGGG | 1518 | CCCTAACCACTCAGGATTGGGCCGT |
| 255 | AACGGCCCAATCCTGAGTGGTTAGG | 1519 | CCTAACCACTCAGGATTGGGCCGTT |
| 256 | AAACGGCCCAATCCTGAGTGGTTAG | 1520 | CTAACCACTCAGGATTGGGCCGTTT |
| 257 | CAAACGGCCCAATCCTGAGTGGTTA | 1521 | TAACCACTCAGGATTGGGCCGTTTG |
| 258 | ACAAACGGCCCAATCCTGAGTGGTT | 1522 | AACCACTCAGGATTGGGCCGTTTGT |
| 259 | CACAAACGGCCCAATCCTGAGTGGT | 1523 | ACCACTCAGGATTGGGCCGTTTGTG |
| 260 | ACACAAACGGCCCAATCCTGAGTGG | 1524 | CCACTCAGGATTGGGCCGTTTGTGT |
| 261 | GACACAAACGGCCCAATCCTGAGTG | 1525 | CACTCAGGATTGGGCCGTTTGTGTC |
| 262 | AGACACAAACGGCCCAATCCTGAGT | 1526 | ACTCAGGATTGGGCCGTTTGTGTCT |
| 263 | CAGACACAAACGGCCCAATCCTGAG | 1527 | CTCAGGATTGGGCCGTTTGTGTCTG |
| 264 | CCAGACACAAACGGCCCAATCCTGA | 1528 | TCAGGATTGGGCCGTTTGTGTCTGG |
| 265 | CCCAGACACAAACGGCCCAATCCTG | 1529 | CAGGATTGGGCCGTTTGTGTCTGGG |
| 266 | ACCCAGACACAAACGGCCCAATCCT | 1530 | AGGATTGGGCCGTTTGTGTCTGGGT |
| 267 | TACCCAGACACAAACGGCCCAATCC | 1531 | GGATTGGGCCGTTTGTGTCTGGGTA |
| 268 | ATACCCAGACACAAACGGCCCAATC | 1532 | GATTGGGCCGTTTGTGTCTGGGTAT |
| 269 | CATACCCAGACACAAACGGCCCAAT | 1533 | ATTGGGCCGTTTGTGTCTGGGTATG |
| 270 | ACATACCCAGACACAAACGGCCCAA | 1534 | TTGGGCCGTTTGTGTCTGGGTATGT |
| 271 | GACATACCCAGACACAAACGGCCCA | 1535 | TGGGCCGTTTGTGTCTGGGTATGTC |
| 272 | AGACATACCCAGACACAAACGGCCC | 1536 | GGGCCGTTTGTGTCTGGGTATGTCT |
| 273 | GAGACATACCCAGACACAAACGGCC | 1537 | GGCCGTTTGTGTCTGGGTATGTCTC |
| 274 | AGAGACATACCCAGACACAAACGGC | 1538 | GCCGTTTGTGTCTGGGTATGTCTCT |
| 275 | AAGAGACATACCCAGACACAAACGG | 1539 | CCGTTTGTGTCTGGGTATGTCTCTT |
| 276 | GAAGAGACATACCCAGACACAAACG | 1540 | CGTTTGTGTCTGGGTATGTCTCTTC |
| 277 | GGAAGAGACATACCCAGACACAAAC | 1541 | GTTTGTGTCTGGGTATGTCTCTTCC |
| 278 | TGGAAGAGACATACCCAGACACAAA | 1542 | TTTGTGTCTGGGTATGTCTCTTCCA |
| 279 | CTGGAAGAGACATACCCAGACACAA | 1543 | TTGTGTCTGGGTATGTCTCTTCCAG |
| 280 | GCTGGAAGAGACATACCCAGACACA | 1544 | TGTGTCTGGGTATGTCTCTTCCAGC |
| 281 | AGCTGGAAGAGACATACCCAGACAC | 1545 | GTGTCTGGGTATGTCTCTTCCAGCT |
| 282 | CAGCTGGAAGAGACATACCCAGACA | 1546 | TGTCTGGGTATGTCTCTTCCAGCTG |
| 283 | GCAGCTGGAAGAGACATACCCAGAC | 1547 | GTCTGGGTATGTCTCTTCCAGCTGC |
| 284 | GGCAGCTGGAAGAGACATACCCAGA | 1548 | TCTGGGTATGTCTCTTCCAGCTGCC |
| 285 | AGGCAGCTGGAAGAGACATACCCAG | 1549 | CTGGGTATGTCTCTTCCAGCTGCCT |
| 286 | CAGGCAGCTGGAAGAGACATACCCA | 1550 | TGGGTATGTCTCTTCCAGCTGCCTG |
| 287 | CCAGGCAGCTGGAAGAGACATACCC | 1551 | GGGTATGTCTCTTCCAGCTGCCTGG |
| 288 | CCCAGGCAGCTGGAAGAGACATACC | 1552 | GGTATGTCTCTTCCAGCTGCCTGGG |
| 289 | ACCCAGGCAGCTGGAAGAGACATAC | 1553 | GTATGTCTCTTCCAGCTGCCTGGGT |
| 290 | AACCCAGGCAGCTGGAAGAGACATA | 1554 | TATGTCTCTTCCAGCTGCCTGGGTT |
| 291 | AAACCCAGGCAGCTGGAAGAGACAT | 1555 | ATGTCTCTTCCAGCTGCCTGGGTTT |
| 292 | GAAACCCAGGCAGCTGGAAGAGACA | 1556 | TGTCTCTTCCAGCTGCCTGGGTTTC |
| 293 | GGAAACCCAGGCAGCTGGAAGAGAC | 1557 | GTCTCTTCCAGCTGCCTGGGTTTCC |
| 294 | AGGAAACCCAGGCAGCTGGAAGAGA | 1558 | TCTCTTCCAGCTGCCTGGGTTTCCT |
| 295 | CAGGAAACCCAGGCAGCTGGAAGAG | 1559 | CTCTTCCAGCTGCCTGGGTTTCCTG |
| 296 | CCAGGAAACCCAGGCAGCTGGAAGA | 1560 | TCTTCCAGCTGCCTGGGTTTCCTGG |
| 297 | TCCAGGAAACCCAGGCAGCTGGAAG | 1561 | CTTCCAGCTGCCTGGGTTTCCTGGA |
| 298 | TTCCAGGAAACCCAGGCAGCTGGAA | 1562 | TTCCAGCTGCCTGGGTTTCCTGGAA |
| 299 | TTTCCAGGAAACCCAGGCAGCTGGA | 1563 | TCCAGCTGCCTGGGTTTCCTGGAAA |
| 300 | CTTTCCAGGAAACCCAGGCAGCTGG | 1564 | CCAGCTGCCTGGGTTTCCTGGAAAG |
| 301 | TCTTTCCAGGAAACCCAGGCAGCTG | 1565 | CAGCTGCCTGGGTTTCCTGGAAAGA |
| 302 | TTCTTTCCAGGAAACCCAGGCAGCT | 1566 | AGCTGCCTGGGTTTCCTGGAAAGAA |
| 303 | GTTCTTTCCAGGAAACCCAGGCAGC | 1567 | GCTGCCTGGGTTTCCTGGAAAGAAC |
| 304 | AGTTCTTTCCAGGAAACCCAGGCAG | 1568 | CTGCCTGGGTTTCCTGGAAAGAACT |
| 305 | GAGTTCTTTCCAGGAAACCCAGGCA | 1569 | TGCCTGGGTTTCCTGGAAAGAACTC |
| 306 | AGAGTTCTTTCCAGGAAACCCAGGC | 1570 | GCCTGGGTTTCCTGGAAAGAACTCT |
| 307 | AAGAGTTCTTTCCAGGAAACCCAGG | 1571 | CCTGGGTTTCCTGGAAAGAACTCTT |
| 308 | TAAGAGTTCTTTCCAGGAAACCCAG | 1572 | CTGGGTTTCCTGGAAAGAACTCTTA |
| 309 | ATAAGAGTTCTTTCCAGGAAACCCA | 1573 | TGGGTTTCCTGGAAAGAACTCTTAT |
| 310 | GATAAGAGTTCTTTCCAGGAAACCC | 1574 | GGGTTTCCTGGAAAGAACTCTTATC |
| 311 | GGATAAGAGTTCTTTCCAGGAAACC | 1575 | GGTTTCCTGGAAAGAACTCTTATCC |
| 312 | GGGATAAGAGTTCTTTCCAGGAAAC | 1576 | GTTTCCTGGAAAGAACTCTTATCCC |
| 313 | GGGGATAAGAGTTCTTTCCAGGAAA | 1577 | TTTCCTGGAAAGAACTCTTATCCCC |
| 314 | TGGGGATAAGAGTTCTTTCCAGGAA | 1578 | TTCCTGGAAAGAACTCTTATCCCCA |
| 315 | CTGGGGATAAGAGTTCTTTCCAGGA | 1579 | TCCTGGAAAGAACTCTTATCCCCAG |
| 316 | CCTGGGGATAAGAGTTCTTTCCAGG | 1580 | CCTGGAAAGAACTCTTATCCCCAGG |
| 317 | TCCTGGGGATAAGAGTTCTTTCCAG | 1581 | CTGGAAAGAACTCTTATCCCCAGGA |
| 318 | TTCCTGGGGATAAGAGTTCTTTCCA | 1582 | TGGAAAGAACTCTTATCCCCAGGAA |
| 319 | GTTCCTGGGGATAAGAGTTCTTTCC | 1583 | GGAAAGAACTCTTATCCCCAGGAAC |
| 320 | AGTTCCTGGGGATAAGAGTTCTTTC | 1584 | GAAAGAACTCTTATCCCCAGGAACT |
| 321 | TAGTTCCTGGGGATAAGAGTTCTTT | 1585 | AAAGAACTCTTATCCCCAGGAACTA |
| 322 | CTAGTTCCTGGGGATAAGAGTTCTT | 1586 | AAGAACTCTTATCCCCAGGAACTAG |
| 323 | ACTAGTTCCTGGGGATAAGAGTTCT | 1587 | AGAACTCTTATCCCCAGGAACTAGT |
| 324 | AACTAGTTCCTGGGGATAAGAGTTC | 1588 | GAACTCTTATCCCCAGGAACTAGTT |
| 325 | AAACTAGTTCCTGGGGATAAGAGTT | 1589 | AACTCTTATCCCCAGGAACTAGTTT |
| 326 | CAAACTAGTTCCTGGGGATAAGAGT | 1590 | ACTCTTATCCCCAGGAACTAGTTTG |
| 327 | ACAAACTAGTTCCTGGGGATAAGAG | 1591 | CTCTTATCCCCAGGAACTAGTTTGT |
| 328 | AACAAACTAGTTCCTGGGGATAAGA | 1592 | TCTTATCCCCAGGAACTAGTTTGTT |
| 329 | CAACAAACTAGTTCCTGGGGATAAG | 1593 | CTTATCCCCAGGAACTAGTTTGTTG |
| 330 | TCAACAAACTAGTTCCTGGGGATAA | 1594 | TTATCCCCAGGAACTAGTTTGTTGA |
| 331 | TTCAACAAACTAGTTCCTGGGGATA | 1595 | TATCCCCAGGAACTAGTTTGTTGAA |
| 332 | ATTCAACAAACTAGTTCCTGGGGAT | 1596 | ATCCCCAGGAACTAGTTTGTTGAAT |
| 333 | TATTCAACAAACTAGTTCCTGGGGA | 1597 | TCCCCAGGAACTAGTTTGTTGAATA |
| 334 | TTATTCAACAAACTAGTTCCTGGGG | 1598 | CCCCAGGAACTAGTTTGTTGAATAA |
| 335 | TTTATTCAACAAACTAGTTCCTGGG | 1599 | CCCAGGAACTAGTTTGTTGAATAAA |
| 336 | ATTTATTCAACAAACTAGTTCCTGG | 1600 | CCAGGAACTAGTTTGTTGAATAAAT |
| 337 | CATTTATTCAACAAACTAGTTCCTG | 1601 | CAGGAACTAGTTTGTTGAATAAATG |
| 338 | GCATTTATTCAACAAACTAGTTCCT | 1602 | AGGAACTAGTTTGTTGAATAAATGC |
| 339 | AGCATTTATTCAACAAACTAGTTCC | 1603 | GGAACTAGTTTGTTGAATAAATGCT |
| 340 | CAGCATTTATTCAACAAACTAGTTC | 1604 | GAACTAGTTTGTTGAATAAATGCTG |
| 341 | CCAGCATTTATTCAACAAACTAGTT | 1605 | AACTAGTTTGTTGAATAAATGCTGG |
| 342 | ACCAGCATTTATTCAACAAACTAGT | 1606 | ACTAGTTTGTTGAATAAATGCTGGT |
| 343 | CACCAGCATTTATTCAACAAACTAG | 1607 | CTAGTTTGTTGAATAAATGCTGGTG |
| 344 | TCACCAGCATTTATTCAACAAACTA | 1608 | TAGTTTGTTGAATAAATGCTGGTGA |
| 345 | TTCACCAGCATTTATTCAACAAACT | 1609 | AGTTTGTTGAATAAATGCTGGTGAA |
| 346 | ATTCACCAGCATTTATTCAACAAAC | 1610 | GTTTGTTGAATAAATGCTGGTGAAT |
| 347 | CATTCACCAGCATTTATTCAACAAA | 1611 | TTTGTTGAATAAATGCTGGTGAATG |
| 348 | TCATTCACCAGCATTTATTCAACAA | 1612 | TTGTTGAATAAATGCTGGTGAATGA |
| 349 | TTCATTCACCAGCATTTATTCAACA | 1613 | TGTTGAATAAATGCTGGTGAATGAA |
| 350 | ATTCATTCACCAGCATTTATTCAAC | 1614 | GTTGAATAAATGCTGGTGAATGAAT |
| 351 | CATTCATTCACCAGCATTTATTCAA | 1615 | TTGAATAAATGCTGGTGAATGAATG |
| 352 | TCATTCATTCACCAGCATTTATTCA | 1616 | TGAATAAATGCTGGTGAATGAATGA |
| 353 | TTCATTCATTCACCAGCATTTATTC | 1617 | GAATAAATGCTGGTGAATGAATGAA |
| 354 | ATTCATTCATTCACCAGCATTTATT | 1618 | AATAAATGCTGGTGAATGAATGAAT |
| 355 | CATTCATTCATTCACCAGCATTTAT | 1619 | ATAAATGCTGGTGAATGAATGAATG |
| 356 | TCATTCATTCATTCACCAGCATTTA | 1620 | TAAATGCTGGTGAATGAATGAATGA |
| 357 | ATCATTCATTCATTCACCAGCATTT | 1621 | AAATGCTGGTGAATGAATGAATGAT |
| 358 | AATCATTCATTCATTCACCAGCATT | 1622 | AATGCTGGTGAATGAATGAATGATT |
| 359 | CAATCATTCATTCATTCACCAGCAT | 1623 | ATGCTGGTGAATGAATGAATGATTG |
| 360 | TCAATCATTCATTCATTCACCAGCA | 1624 | TGCTGGTGAATGAATGAATGATTGA |
| 361 | TTCAATCATTCATTCATTCACCAGC | 1625 | GCTGGTGAATGAATGAATGATTGAA |
| 362 | GTTCAATCATTCATTCATTCACCAG | 1626 | CTGGTGAATGAATGAATGATTGAAC |
| 363 | TGTTCAATCATTCATTCATTCACCA | 1627 | TGGTGAATGAATGAATGATTGAACA |
| 364 | CTGTTCAATCATTCATTCATTCACC | 1628 | GGTGAATGAATGAATGATTGAACAG |
| 365 | TCTGTTCAATCATTCATTCATTCAC | 1629 | GTGAATGAATGAATGATTGAACAGA |
| 366 | ATCTGTTCAATCATTCATTCATTCA | 1630 | TGAATGAATGAATGATTGAACAGAT |
| 367 | CATCTGTTCAATCATTCATTCATTC | 1631 | GAATGAATGAATGATTGAACAGATG |
| 368 | TCATCTGTTCAATCATTCATTCATT | 1632 | AATGAATGAATGATTGAACAGATGA |
| 369 | TTCATCTGTTCAATCATTCATTCAT | 1633 | ATGAATGAATGATTGAACAGATGAA |
| 370 | ATTCATCTGTTCAATCATTCATTCA | 1634 | TGAATGAATGATTGAACAGATGAAT |
| 371 | CATTCATCTGTTCAATCATTCATTC | 1635 | GAATGAATGATTGAACAGATGAATG |
| 372 | TCATTCATCTGTTCAATCATTCATT | 1636 | AATGAATGATTGAACAGATGAATGA |
| 373 | CTCATTCATCTGTTCAATCATTCAT | 1637 | ATGAATGATTGAACAGATGAATGAG |
| 374 | ACTCATTCATCTGTTCAATCATTCA | 1638 | TGAATGATTGAACAGATGAATGAGT |
| 375 | CACTCATTCATCTGTTCAATCATTC | 1639 | GAATGATTGAACAGATGAATGAGTG |
| 376 | TCACTCATTCATCTGTTCAATCATT | 1640 | AATGATTGAACAGATGAATGAGTGA |
| 377 | ATCACTCATTCATCTGTTCAATCAT | 1641 | ATGATTGAACAGATGAATGAGTGAT |
| 378 | CATCACTCATTCATCTGTTCAATCA | 1642 | TGATTGAACAGATGAATGAGTGATG |
| 379 | TCATCACTCATTCATCTGTTCAATC | 1643 | GATTGAACAGATGAATGAGTGATGA |
| 380 | CTCATCACTCATTCATCTGTTCAAT | 1644 | ATTGAACAGATGAATGAGTGATGAG |
| 381 | ACTCATCACTCATTCATCTGTTCAA | 1645 | TTGAACAGATGAATGAGTGATGAGT |
| 382 | TACTCATCACTCATTCATCTGTTCA | 1646 | TGAACAGATGAATGAGTGATGAGTA |
| 383 | CTACTCATCACTCATTCATCTGTTC | 1647 | GAACAGATGAATGAGTGATGAGTAG |
| 384 | TCTACTCATCACTCATTCATCTGTT | 1648 | AACAGATGAATGAGTGATGAGTAGA |
| 385 | ATCTACTCATCACTCATTCATCTGT | 1649 | ACAGATGAATGAGTGATGAGTAGAT |
| 386 | TATCTACTCATCACTCATTCATCTG | 1650 | CAGATGAATGAGTGATGAGTAGATA |
| 387 | TTATCTACTCATCACTCATTCATCT | 1651 | AGATGAATGAGTGATGAGTAGATAA |
| 388 | TTTATCTACTCATCACTCATTCATC | 1652 | GATGAATGAGTGATGAGTAGATAAA |
| 389 | TTTTATCTACTCATCACTCATTCAT | 1653 | ATGAATGAGTGATGAGTAGATAAAA |
| 390 | CTTTTATCTACTCATCACTCATTCA | 1654 | TGAATGAGTGATGAGTAGATAAAAG |
| 391 | CCTTTTATCTACTCATCACTCATTC | 1655 | GAATGAGTGATGAGTAGATAAAAGG |
| 392 | TCCTTTTATCTACTCATCACTCATT | 1656 | AATGAGTGATGAGTAGATAAAAGGA |
| 393 | ATCCTTTTATCTACTCATCACTCAT | 1657 | ATGAGTGATGAGTAGATAAAAGGAT |
| 394 | CATCCTTTTATCTACTCATCACTCA | 1658 | TGAGTGATGAGTAGATAAAAGGATG |
| 395 | CCATCCTTTTATCTACTCATCACTC | 1659 | GAGTGATGAGTAGATAAAAGGATGG |
| 396 | TCCATCCTTTTATCTACTCATCACT | 1660 | AGTGATGAGTAGATAAAAGGATGGA |
| 397 | ATCCATCCTTTTATCTACTCATCAC | 1661 | GTGATGAGTAGATAAAAGGATGGAT |
| 398 | CATCCATCCTTTTATCTACTCATCA | 1662 | TGATGAGTAGATAAAAGGATGGATG |
| 399 | CCATCCATCCTTTTATCTACTCATC | 1663 | GATGAGTAGATAAAAGGATGGATGG |
| 400 | TCCATCCATCCTTTTATCTACTCAT | 1664 | ATGAGTAGATAAAAGGATGGATGGA |
| 401 | CTCCATCCATCCTTTTATCTACTCA | 1665 | TGAGTAGATAAAAGGATGGATGGAG |
| 402 | TCTCCATCCATCCTTTTATCTACTC | 1666 | GAGTAGATAAAAGGATGGATGGAGA |
| 403 | CTCTCCATCCATCCTTTTATCTACT | 1667 | AGTAGATAAAAGGATGGATGGAGAG |
| 404 | TCTCTCCATCCATCCTTTTATCTAC | 1668 | GTAGATAAAAGGATGGATGGAGAGA |
| 405 | ATCTCTCCATCCATCCTTTTATCTA | 1669 | TAGATAAAAGGATGGATGGAGAGAT |
| 406 | CATCTCTCCATCCATCCTTTTATCT | 1670 | AGATAAAAGGATGGATGGAGAGATG |
| 407 | CCATCTCTCCATCCATCCTTTTATC | 1671 | GATAAAAGGATGGATGGAGAGATGG |
| 408 | CCCATCTCTCCATCCATCCTTTTAT | 1672 | ATAAAAGGATGGATGGAGAGATGGG |
| 409 | ACCCATCTCTCCATCCATCCTTTTA | 1673 | TAAAAGGATGGATGGAGAGATGGGT |
| 410 | CACCCATCTCTCCATCCATCCTTTT | 1674 | AAAAGGATGGATGGAGAGATGGGTG |
| 411 | TCACCCATCTCTCCATCCATCCTTT | 1675 | AAAGGATGGATGGAGAGATGGGTGA |
| 412 | CTCACCCATCTCTCCATCCATCCTT | 1676 | AAGGATGGATGGAGAGATGGGTGAG |
| 413 | ACTCACCCATCTCTCCATCCATCCT | 1677 | AGGATGGATGGAGAGATGGGTGAGT |
| 414 | TACTCACCCATCTCTCCATCCATCC | 1678 | GGATGGATGGAGAGATGGGTGAGTA |
| 415 | GTACTCACCCATCTCTCCATCCATC | 1679 | GATGGATGGAGAGATGGGTGAGTAC |
| 416 | TGTACTCACCCATCTCTCCATCCAT | 1680 | ATGGATGGAGAGATGGGTGAGTACA |
| 417 | ATGTACTCACCCATCTCTCCATCCA | 1681 | TGGATGGAGAGATGGGTGAGTACAT |
| 418 | CATGTACTCACCCATCTCTCCATCC | 1682 | GGATGGAGAGATGGGTGAGTACATG |
| 419 | CCATGTACTCACCCATCTCTCCATC | 1683 | GATGGAGAGATGGGTGAGTACATGG |
| 420 | TCCATGTACTCACCCATCTCTCCAT | 1684 | ATGGAGAGATGGGTGAGTACATGGA |
| 421 | ATCCATGTACTCACCCATCTCTCCA | 1685 | TGGAGAGATGGGTGAGTACATGGAT |
| 422 | CATCCATGTACTCACCCATCTCTCC | 1686 | GGAGAGATGGGTGAGTACATGGATG |
| 423 | CCATCCATGTACTCACCCATCTCTC | 1687 | GAGAGATGGGTGAGTACATGGATGG |
| 424 | TCCATCCATGTACTCACCCATCTCT | 1688 | AGAGATGGGTGAGTACATGGATGGA |
| 425 | ATCCATCCATGTACTCACCCATCTC | 1689 | GAGATGGGTGAGTACATGGATGGAT |
| 426 | TATCCATCCATGTACTCACCCATCT | 1690 | AGATGGGTGAGTACATGGATGGATA |
| 427 | CTATCCATCCATGTACTCACCCATC | 1691 | GATGGGTGAGTACATGGATGGATAG |
| 428 | TCTATCCATCCATGTACTCACCCAT | 1692 | ATGGGTGAGTACATGGATGGATAGA |
| 429 | ATCTATCCATCCATGTACTCACCCA | 1693 | TGGGTGAGTACATGGATGGATAGAT |
| 430 | CATCTATCCATCCATGTACTCACCC | 1694 | GGGTGAGTACATGGATGGATAGATG |
| 431 | CCATCTATCCATCCATGTACTCACC | 1695 | GGTGAGTACATGGATGGATAGATGG |
| 432 | TCCATCTATCCATCCATGTACTCAC | 1696 | GTGAGTACATGGATGGATAGATGGA |
| 433 | ATCCATCTATCCATCCATGTACTCA | 1697 | TGAGTACATGGATGGATAGATGGAT |
| 434 | CATCCATCTATCCATCCATGTACTC | 1698 | GAGTACATGGATGGATAGATGGATG |
| 435 | TCATCCATCTATCCATCCATGTACT | 1699 | AGTACATGGATGGATAGATGGATGA |
| 436 | CTCATCCATCTATCCATCCATGTAC | 1700 | GTACATGGATGGATAGATGGATGAG |
| 437 | ACTCATCCATCTATCCATCCATGTA | 1701 | TACATGGATGGATAGATGGATGAGT |
| 438 | AACTCATCCATCTATCCATCCATGT | 1702 | ACATGGATGGATAGATGGATGAGTT |
| 439 | CAACTCATCCATCTATCCATCCATG | 1703 | CATGGATGGATAGATGGATGAGTTG |
| 440 | CCAACTCATCCATCTATCCATCCAT | 1704 | ATGGATGGATAGATGGATGAGTTGG |
| 441 | ACCAACTCATCCATCTATCCATCCA | 1705 | TGGATGGATAGATGGATGAGTTGGT |
| 442 | CACCAACTCATCCATCTATCCATCC | 1706 | GGATGGATAGATGGATGAGTTGGTG |
| 443 | CCACCAACTCATCCATCTATCCATC | 1707 | GATGGATAGATGGATGAGTTGGTGG |
| 444 | CCCACCAACTCATCCATCTATCCAT | 1708 | ATGGATAGATGGATGAGTTGGTGGG |
| 445 | ACCCACCAACTCATCCATCTATCCA | 1709 | TGGATAGATGGATGAGTTGGTGGGT |
| 446 | TACCCACCAACTCATCCATCTATCC | 1710 | GGATAGATGGATGAGTTGGTGGGTA |
| 447 | CTACCCACCAACTCATCCATCTATC | 1711 | GATAGATGGATGAGTTGGTGGGTAG |
| 448 | TCTACCCACCAACTCATCCATCTAT | 1712 | ATAGATGGATGAGTTGGTGGGTAGA |
| 449 | ATCTACCCACCAACTCATCCATCTA | 1713 | TAGATGGATGAGTTGGTGGGTAGAT |
| 450 | AATCTACCCACCAACTCATCCATCT | 1714 | AGATGGATGAGTTGGTGGGTAGATT |
| 451 | GAATCTACCCACCAACTCATCCATC | 1715 | GATGGATGAGTTGGTGGGTAGATTC |
| 452 | CGAATCTACCCACCAACTCATCCAT | 1716 | ATGGATGAGTTGGTGGGTAGATTCG |
| 453 | ACGAATCTACCCACCAACTCATCCA | 1717 | TGGATGAGTTGGTGGGTAGATTCGT |
| 454 | CACGAATCTACCCACCAACTCATCC | 1718 | GGATGAGTTGGTGGGTAGATTCGTG |
| 455 | CCACGAATCTACCCACCAACTCATC | 1719 | GATGAGTTGGTGGGTAGATTCGTGG |
| 456 | GCCACGAATCTACCCACCAACTCAT | 1720 | ATGAGTTGGTGGGTAGATTCGTGGC |
| 457 | AGCCACGAATCTACCCACCAACTCA | 1721 | TGAGTTGGTGGGTAGATTCGTGGCT |
| 458 | TAGCCACGAATCTACCCACCAACTC | 1722 | GAGTTGGTGGGTAGATTCGTGGCTA |
| 459 | CTAGCCACGAATCTACCCACCAACT | 1723 | AGTTGGTGGGTAGATTCGTGGCTAG |
| 460 | TCTAGCCACGAATCTACCCACCAAC | 1724 | GTTGGTGGGTAGATTCGTGGCTAGA |
| 461 | ATCTAGCCACGAATCTACCCACCAA | 1725 | TTGGTGGGTAGATTCGTGGCTAGAT |
| 462 | CATCTAGCCACGAATCTACCCACCA | 1726 | TGGTGGGTAGATTCGTGGCTAGATG |
| 463 | CCATCTAGCCACGAATCTACCCACC | 1727 | GGTGGGTAGATTCGTGGCTAGATGG |
| 464 | TCCATCTAGCCACGAATCTACCCAC | 1728 | GTGGGTAGATTCGTGGCTAGATGGA |
| 465 | ATCCATCTAGCCACGAATCTACCCA | 1729 | TGGGTAGATTCGTGGCTAGATGGAT |
| 466 | CATCCATCTAGCCACGAATCTACCC | 1730 | GGGTAGATTCGTGGCTAGATGGATG |
| 467 | TCATCCATCTAGCCACGAATCTACC | 1731 | GGTAGATTCGTGGCTAGATGGATGA |
| 468 | ATCATCCATCTAGCCACGAATCTAC | 1732 | GTAGATTCGTGGCTAGATGGATGAT |
| 469 | CATCATCCATCTAGCCACGAATCTA | 1733 | TAGATTCGTGGCTAGATGGATGATG |
| 470 | CCATCATCCATCTAGCCACGAATCT | 1734 | AGATTCGTGGCTAGATGGATGATGG |
| 471 | TCCATCATCCATCTAGCCACGAATC | 1735 | GATTCGTGGCTAGATGGATGATGGA |
| 472 | ATCCATCATCCATCTAGCCACGAAT | 1736 | ATTCGTGGCTAGATGGATGATGGAT |
| 473 | CATCCATCATCCATCTAGCCACGAA | 1737 | TTCGTGGCTAGATGGATGATGGATG |
| 474 | CCATCCATCATCCATCTAGCCACGA | 1738 | TCGTGGCTAGATGGATGATGGATGG |
| 475 | TCCATCCATCATCCATCTAGCCACG | 1739 | CGTGGCTAGATGGATGATGGATGGA |
| 476 | ATCCATCCATCATCCATCTAGCCAC | 1740 | GTGGCTAGATGGATGATGGATGGAT |
| 477 | CATCCATCCATCATCCATCTAGCCA | 1741 | TGGCTAGATGGATGATGGATGGATG |
| 478 | CCATCCATCCATCATCCATCTAGCC | 1742 | GGCTAGATGGATGATGGATGGATGG |
| 479 | TCCATCCATCCATCATCCATCTAGC | 1743 | GCTAGATGGATGATGGATGGATGGA |
| 480 | GTCCATCCATCCATCATCCATCTAG | 1744 | CTAGATGGATGATGGATGGATGGAC |
| 481 | TGTCCATCCATCCATCATCCATCTA | 1745 | TAGATGGATGATGGATGGATGGACA |
| 482 | CTGTCCATCCATCCATCATCCATCT | 1746 | AGATGGATGATGGATGGATGGACAG |
| 483 | TCTGTCCATCCATCCATCATCCATC | 1747 | GATGGATGATGGATGGATGGACAGA |
| 484 | ATCTGTCCATCCATCCATCATCCAT | 1748 | ATGGATGATGGATGGATGGACAGAT |
| 485 | CATCTGTCCATCCATCCATCATCCA | 1749 | TGGATGATGGATGGATGGACAGATG |
| 486 | CCATCTGTCCATCCATCCATCATCC | 1750 | GGATGATGGATGGATGGACAGATGG |
| 487 | TCCATCTGTCCATCCATCCATCATC | 1751 | GATGATGGATGGATGGACAGATGGA |
| 488 | ATCCATCTGTCCATCCATCCATCAT | 1752 | ATGATGGATGGATGGACAGATGGAT |
| 489 | CATCCATCTGTCCATCCATCCATCA | 1753 | TGATGGATGGATGGACAGATGGATG |
| 490 | CCATCCATCTGTCCATCCATCCATC | 1754 | GATGGATGGATGGACAGATGGATGG |
| 491 | TCCATCCATCTGTCCATCCATCCAT | 1755 | ATGGATGGATGGACAGATGGATGGA |
| 492 | ATCCATCCATCTGTCCATCCATCCA | 1756 | TGGATGGATGGACAGATGGATGGAT |
| 493 | TATCCATCCATCTGTCCATCCATCC | 1757 | GGATGGATGGACAGATGGATGGATA |
| 494 | ATATCCATCCATCTGTCCATCCATC | 1758 | GATGGATGGACAGATGGATGGATAT |
| 495 | TATATCCATCCATCTGTCCATCCAT | 1759 | ATGGATGGACAGATGGATGGATATA |
| 496 | ATATATCCATCCATCTGTCCATCCA | 1760 | TGGATGGACAGATGGATGGATATAT |
| 497 | CATATATCCATCCATCTGTCCATCC | 1761 | GGATGGACAGATGGATGGATATATG |
| 498 | TCATATATCCATCCATCTGTCCATC | 1762 | GATGGACAGATGGATGGATATATGA |
| 499 | ATCATATATCCATCCATCTGTCCAT | 1763 | ATGGACAGATGGATGGATATATGAT |
| 500 | AATCATATATCCATCCATCTGTCCA | 1764 | TGGACAGATGGATGGATATATGATT |
| 501 | CAATCATATATCCATCCATCTGTCC | 1765 | GGACAGATGGATGGATATATGATTG |
| 502 | TCAATCATATATCCATCCATCTGTC | 1766 | GACAGATGGATGGATATATGATTGA |
| 503 | TTCAATCATATATCCATCCATCTGT | 1767 | ACAGATGGATGGATATATGATTGAA |
| 504 | GTTCAATCATATATCCATCCATCTG | 1768 | CAGATGGATGGATATATGATTGAAC |
| 505 | AGTTCAATCATATATCCATCCATCT | 1769 | AGATGGATGGATATATGATTGAACT |
| 506 | TAGTTCAATCATATATCCATCCATC | 1770 | GATGGATGGATATATGATTGAACTA |
| 507 | ATAGTTCAATCATATATCCATCCAT | 1771 | ATGGATGGATATATGATTGAACTAT |
| 508 | AATAGTTCAATCATATATCCATCCA | 1772 | TGGATGGATATATGATTGAACTATT |
| 509 | CAATAGTTCAATCATATATCCATCC | 1773 | GGATGGATATATGATTGAACTATTG |
| 510 | TCAATAGTTCAATCATATATCCATC | 1774 | GATGGATATATGATTGAACTATTGA |
| 511 | TTCAATAGTTCAATCATATATCCAT | 1775 | ATGGATATATGATTGAACTATTGAA |
| 512 | TTTCAATAGTTCAATCATATATCCA | 1776 | TGGATATATGATTGAACTATTGAAA |
| 513 | CTTTCAATAGTTCAATCATATATCC | 1777 | GGATATATGATTGAACTATTGAAAG |
| 514 | ACTTTCAATAGTTCAATCATATATC | 1778 | GATATATGATTGAACTATTGAAAGT |
| 515 | TACTTTCAATAGTTCAATCATATAT | 1779 | ATATATGATTGAACTATTGAAAGTA |
| 516 | ATACTTTCAATAGTTCAATCATATA | 1780 | TATATGATTGAACTATTGAAAGTAT |
| 517 | TATACTTTCAATAGTTCAATCATAT | 1781 | ATATGATTGAACTATTGAAAGTATA |
| 518 | CTATACTTTCAATAGTTCAATCATA | 1782 | TATGATTGAACTATTGAAAGTATAG |
| 519 | TCTATACTTTCAATAGTTCAATCAT | 1783 | ATGATTGAACTATTGAAAGTATAGA |
| 520 | ATCTATACTTTCAATAGTTCAATCA | 1784 | TGATTGAACTATTGAAAGTATAGAT |
| 521 | CATCTATACTTTCAATAGTTCAATC | 1785 | GATTGAACTATTGAAAGTATAGATG |
| 522 | ACATCTATACTTTCAATAGTTCAAT | 1786 | ATTGAACTATTGAAAGTATAGATGT |
| 523 | TACATCTATACTTTCAATAGTTCAA | 1787 | TTGAACTATTGAAAGTATAGATGTA |
| 524 | ATACATCTATACTTTCAATAGTTCA | 1788 | TGAACTATTGAAAGTATAGATGTAT |
| 525 | CATACATCTATACTTTCAATAGTTC | 1789 | GAACTATTGAAAGTATAGATGTATG |
| 526 | CCATACATCTATACTTTCAATAGTT | 1790 | AACTATTGAAAGTATAGATGTATGG |
| 527 | TCCATACATCTATACTTTCAATAGT | 1791 | ACTATTGAAAGTATAGATGTATGGA |
| 528 | ATCCATACATCTATACTTTCAATAG | 1792 | CTATTGAAAGTATAGATGTATGGAT |
| 529 | CATCCATACATCTATACTTTCAATA | 1793 | TATTGAAAGTATAGATGTATGGATG |
| 530 | CCATCCATACATCTATACTTTCAAT | 1794 | ATTGAAAGTATAGATGTATGGATGG |
| 531 | CCCATCCATACATCTATACTTTCAA | 1795 | TTGAAAGTATAGATGTATGGATGGG |
| 532 | ACCCATCCATACATCTATACTTTCA | 1796 | TGAAAGTATAGATGTATGGATGGGT |
| 533 | CACCCATCCATACATCTATACTTTC | 1797 | GAAAGTATAGATGTATGGATGGGTG |
| 534 | TCACCCATCCATACATCTATACTTT | 1798 | AAAGTATAGATGTATGGATGGGTGA |
| 535 | TTCACCCATCCATACATCTATACTT | 1799 | AAGTATAGATGTATGGATGGGTGAA |
| 536 | ATTCACCCATCCATACATCTATACT | 1800 | AGTATAGATGTATGGATGGGTGAAT |
| 537 | AATTCACCCATCCATACATCTATAC | 1801 | GTATAGATGTATGGATGGGTGAATT |
| 538 | AAATTCACCCATCCATACATCTATA | 1802 | TATAGATGTATGGATGGGTGAATTT |
| 539 | CAAATTCACCCATCCATACATCTAT | 1803 | ATAGATGTATGGATGGGTGAATTTG |
| 540 | CCAAATTCACCCATCCATACATCTA | 1804 | TAGATGTATGGATGGGTGAATTTGG |
| 541 | CCCAAATTCACCCATCCATACATCT | 1805 | AGATGTATGGATGGGTGAATTTGGG |
| 542 | CCCCAAATTCACCCATCCATACATC | 1806 | GATGTATGGATGGGTGAATTTGGGG |
| 543 | CCCCCAAATTCACCCATCCATACAT | 1807 | ATGTATGGATGGGTGAATTTGGGGG |
| 544 | ACCCCCAAATTCACCCATCCATACA | 1808 | TGTATGGATGGGTGAATTTGGGGGT |
| 545 | TACCCCCAAATTCACCCATCCATAC | 1809 | GTATGGATGGGTGAATTTGGGGGTA |
| 546 | TTACCCCCAAATTCACCCATCCATA | 1810 | TATGGATGGGTGAATTTGGGGGTAA |
| 547 | ATTACCCCCAAATTCACCCATCCAT | 1811 | ATGGATGGGTGAATTTGGGGGTAAT |
| 548 | AATTACCCCCAAATTCACCCATCCA | 1812 | TGGATGGGTGAATTTGGGGGTAATT |
| 549 | CAATTACCCCCAAATTCACCCATCC | 1813 | GGATGGGTGAATTTGGGGGTAATTG |
| 550 | ACAATTACCCCCAAATTCACCCATC | 1814 | GATGGGTGAATTTGGGGGTAATTGT |
| 551 | AACAATTACCCCCAAATTCACCCAT | 1815 | ATGGGTGAATTTGGGGGTAATTGTT |
| 552 | TAACAATTACCCCCAAATTCACCCA | 1816 | TGGGTGAATTTGGGGGTAATTGTTA |
| 553 | CTAACAATTACCCCCAAATTCACCC | 1817 | GGGTGAATTTGGGGGTAATTGTTAG |
| 554 | TCTAACAATTACCCCCAAATTCACC | 1818 | GGTGAATTTGGGGGTAATTGTTAGA |
| 555 | ATCTAACAATTACCCCCAAATTCAC | 1819 | GTGAATTTGGGGGTAATTGTTAGAT |
| 556 | CATCTAACAATTACCCCCAAATTCA | 1820 | TGAATTTGGGGGTAATTGTTAGATG |
| 557 | TCATCTAACAATTACCCCCAAATTC | 1821 | GAATTTGGGGGTAATTGTTAGATGA |
| 558 | ATCATCTAACAATTACCCCCAAATT | 1822 | AATTTGGGGGTAATTGTTAGATGAT |
| 559 | CATCATCTAACAATTACCCCCAAAT | 1823 | ATTTGGGGGTAATTGTTAGATGATG |
| 560 | CCATCATCTAACAATTACCCCCAAA | 1824 | TTTGGGGGTAATTGTTAGATGATGG |
| 561 | TCCATCATCTAACAATTACCCCCAA | 1825 | TTGGGGGTAATTGTTAGATGATGGA |
| 562 | ATCCATCATCTAACAATTACCCCCA | 1826 | TGGGGGTAATTGTTAGATGATGGAT |
| 563 | CATCCATCATCTAACAATTACCCCC | 1827 | GGGGGTAATTGTTAGATGATGGATG |
| 564 | TCATCCATCATCTAACAATTACCCC | 1828 | GGGGTAATTGTTAGATGATGGATGA |
| 565 | CTCATCCATCATCTAACAATTACCC | 1829 | GGGTAATTGTTAGATGATGGATGAG |
| 566 | ACTCATCCATCATCTAACAATTACC | 1830 | GGTAATTGTTAGATGATGGATGAGT |
| 567 | TACTCATCCATCATCTAACAATTAC | 1831 | GTAATTGTTAGATGATGGATGAGTA |
| 568 | ATACTCATCCATCATCTAACAATTA | 1832 | TAATTGTTAGATGATGGATGAGTAT |
| 569 | TATACTCATCCATCATCTAACAATT | 1833 | AATTGTTAGATGATGGATGAGTATA |
| 570 | CTATACTCATCCATCATCTAACAAT | 1834 | ATTGTTAGATGATGGATGAGTATAG |
| 571 | TCTATACTCATCCATCATCTAACAA | 1835 | TTGTTAGATGATGGATGAGTATAGA |
| 572 | ATCTATACTCATCCATCATCTAACA | 1836 | TGTTAGATGATGGATGAGTATAGAT |
| 573 | CATCTATACTCATCCATCATCTAAC | 1837 | GTTAGATGATGGATGAGTATAGATG |
| 574 | TCATCTATACTCATCCATCATCTAA | 1838 | TTAGATGATGGATGAGTATAGATGA |
| 575 | TTCATCTATACTCATCCATCATCTA | 1839 | TAGATGATGGATGAGTATAGATGAA |
| 576 | ATTCATCTATACTCATCCATCATCT | 1840 | AGATGATGGATGAGTATAGATGAAT |
| 577 | CATTCATCTATACTCATCCATCATC | 1841 | GATGATGGATGAGTATAGATGAATG |
| 578 | TCATTCATCTATACTCATCCATCAT | 1842 | ATGATGGATGAGTATAGATGAATGA |
| 579 | ATCATTCATCTATACTCATCCATCA | 1843 | TGATGGATGAGTATAGATGAATGAT |
| 580 | CATCATTCATCTATACTCATCCATC | 1844 | GATGGATGAGTATAGATGAATGATG |
| 581 | CCATCATTCATCTATACTCATCCAT | 1845 | ATGGATGAGTATAGATGAATGATGG |
| 582 | TCCATCATTCATCTATACTCATCCA | 1846 | TGGATGAGTATAGATGAATGATGGA |
| 583 | ATCCATCATTCATCTATACTCATCC | 1847 | GGATGAGTATAGATGAATGATGGAT |
| 584 | CATCCATCATTCATCTATACTCATC | 1848 | GATGAGTATAGATGAATGATGGATG |
| 585 | CCATCCATCATTCATCTATACTCAT | 1849 | ATGAGTATAGATGAATGATGGATGG |
| 586 | TCCATCCATCATTCATCTATACTCA | 1850 | TGAGTATAGATGAATGATGGATGGA |
| 587 | ATCCATCCATCATTCATCTATACTC | 1851 | GAGTATAGATGAATGATGGATGGAT |
| 588 | TATCCATCCATCATTCATCTATACT | 1852 | AGTATAGATGAATGATGGATGGATA |
| 589 | TTATCCATCCATCATTCATCTATAC | 1853 | GTATAGATGAATGATGGATGGATAA |
| 590 | GTTATCCATCCATCATTCATCTATA | 1854 | TATAGATGAATGATGGATGGATAAC |
| 591 | AGTTATCCATCCATCATTCATCTAT | 1855 | ATAGATGAATGATGGATGGATAACT |
| 592 | AAGTTATCCATCCATCATTCATCTA | 1856 | TAGATGAATGATGGATGGATAACTT |
| 593 | CAAGTTATCCATCCATCATTCATCT | 1857 | AGATGAATGATGGATGGATAACTTG |
| 594 | TCAAGTTATCCATCCATCATTCATC | 1858 | GATGAATGATGGATGGATAACTTGA |
| 595 | ATCAAGTTATCCATCCATCATTCAT | 1859 | ATGAATGATGGATGGATAACTTGAT |
| 596 | CATCAAGTTATCCATCCATCATTCA | 1860 | TGAATGATGGATGGATAACTTGATG |
| 597 | TCATCAAGTTATCCATCCATCATTC | 1861 | GAATGATGGATGGATAACTTGATGA |
| 598 | CTCATCAAGTTATCCATCCATCATT | 1862 | AATGATGGATGGATAACTTGATGAG |
| 599 | ACTCATCAAGTTATCCATCCATCAT | 1863 | ATGATGGATGGATAACTTGATGAGT |
| 600 | CACTCATCAAGTTATCCATCCATCA | 1864 | TGATGGATGGATAACTTGATGAGTG |
| 601 | CCACTCATCAAGTTATCCATCCATC | 1865 | GATGGATGGATAACTTGATGAGTGG |
| 602 | TCCACTCATCAAGTTATCCATCCAT | 1866 | ATGGATGGATAACTTGATGAGTGGA |
| 603 | ATCCACTCATCAAGTTATCCATCCA | 1867 | TGGATGGATAACTTGATGAGTGGAT |
| 604 | TATCCACTCATCAAGTTATCCATCC | 1868 | GGATGGATAACTTGATGAGTGGATA |
| 605 | CTATCCACTCATCAAGTTATCCATC | 1869 | GATGGATAACTTGATGAGTGGATAG |
| 606 | TCTATCCACTCATCAAGTTATCCAT | 1870 | ATGGATAACTTGATGAGTGGATAGA |
| 607 | ATCTATCCACTCATCAAGTTATCCA | 1871 | TGGATAACTTGATGAGTGGATAGAT |
| 608 | TATCTATCCACTCATCAAGTTATCC | 1872 | GGATAACTTGATGAGTGGATAGATA |
| 609 | CTATCTATCCACTCATCAAGTTATC | 1873 | GATAACTTGATGAGTGGATAGATAG |
| 610 | TCTATCTATCCACTCATCAAGTTAT | 1874 | ATAACTTGATGAGTGGATAGATAGA |
| 611 | ATCTATCTATCCACTCATCAAGTTA | 1875 | TAACTTGATGAGTGGATAGATAGAT |
| 612 | AATCTATCTATCCACTCATCAAGTT | 1876 | AACTTGATGAGTGGATAGATAGATT |
| 613 | CAATCTATCTATCCACTCATCAAGT | 1877 | ACTTGATGAGTGGATAGATAGATTG |
| 614 | GCAATCTATCTATCCACTCATCAAG | 1878 | CTTGATGAGTGGATAGATAGATTGC |
| 615 | AGCAATCTATCTATCCACTCATCAA | 1879 | TTGATGAGTGGATAGATAGATTGCT |
| 616 | CAGCAATCTATCTATCCACTCATCA | 1880 | TGATGAGTGGATAGATAGATTGCTG |
| 617 | CCAGCAATCTATCTATCCACTCATC | 1881 | GATGAGTGGATAGATAGATTGCTGG |
| 618 | TCCAGCAATCTATCTATCCACTCAT | 1882 | ATGAGTGGATAGATAGATTGCTGGA |
| 619 | ATCCAGCAATCTATCTATCCACTCA | 1883 | TGAGTGGATAGATAGATTGCTGGAT |
| 620 | TATCCAGCAATCTATCTATCCACTC | 1884 | GAGTGGATAGATAGATTGCTGGATA |
| 621 | CTATCCAGCAATCTATCTATCCACT | 1885 | AGTGGATAGATAGATTGCTGGATAG |
| 622 | TCTATCCAGCAATCTATCTATCCAC | 1886 | GTGGATAGATAGATTGCTGGATAGA |
| 623 | ATCTATCCAGCAATCTATCTATCCA | 1887 | TGGATAGATAGATTGCTGGATAGAT |
| 624 | CATCTATCCAGCAATCTATCTATCC | 1888 | GGATAGATAGATTGCTGGATAGATG |
| 625 | TCATCTATCCAGCAATCTATCTATC | 1889 | GATAGATAGATTGCTGGATAGATGA |
| 626 | ATCATCTATCCAGCAATCTATCTAT | 1890 | ATAGATAGATTGCTGGATAGATGAT |
| 627 | AATCATCTATCCAGCAATCTATCTA | 1891 | TAGATAGATTGCTGGATAGATGATT |
| 628 | CAATCATCTATCCAGCAATCTATCT | 1892 | AGATAGATTGCTGGATAGATGATTG |
| 629 | TCAATCATCTATCCAGCAATCTATC | 1893 | GATAGATTGCTGGATAGATGATTGA |
| 630 | GTCAATCATCTATCCAGCAATCTAT | 1894 | ATAGATTGCTGGATAGATGATTGAC |
| 631 | AGTCAATCATCTATCCAGCAATCTA | 1895 | TAGATTGCTGGATAGATGATTGACT |
| 632 | CAGTCAATCATCTATCCAGCAATCT | 1896 | AGATTGCTGGATAGATGATTGACTG |
| 633 | CCAGTCAATCATCTATCCAGCAATC | 1897 | GATTGCTGGATAGATGATTGACTGG |
| 634 | CCCAGTCAATCATCTATCCAGCAAT | 1898 | ATTGCTGGATAGATGATTGACTGGG |
| 635 | ACCCAGTCAATCATCTATCCAGCAA | 1899 | TTGCTGGATAGATGATTGACTGGGT |
| 636 | CACCCAGTCAATCATCTATCCAGCA | 1900 | TGCTGGATAGATGATTGACTGGGTG |
| 637 | CCACCCAGTCAATCATCTATCCAGC | 1901 | GCTGGATAGATGATTGACTGGGTGG |
| 638 | TCCACCCAGTCAATCATCTATCCAG | 1902 | CTGGATAGATGATTGACTGGGTGGA |
| 639 | ATCCACCCAGTCAATCATCTATCCA | 1903 | TGGATAGATGATTGACTGGGTGGAT |
| 640 | TATCCACCCAGTCAATCATCTATCC | 1904 | GGATAGATGATTGACTGGGTGGATA |
| 641 | CTATCCACCCAGTCAATCATCTATC | 1905 | GATAGATGATTGACTGGGTGGATAG |
| 642 | TCTATCCACCCAGTCAATCATCTAT | 1906 | ATAGATGATTGACTGGGTGGATAGA |
| 643 | ATCTATCCACCCAGTCAATCATCTA | 1907 | TAGATGATTGACTGGGTGGATAGAT |
| 644 | CATCTATCCACCCAGTCAATCATCT | 1908 | AGATGATTGACTGGGTGGATAGATG |
| 645 | TCATCTATCCACCCAGTCAATCATC | 1909 | GATGATTGACTGGGTGGATAGATGA |
| 646 | TTCATCTATCCACCCAGTCAATCAT | 1910 | ATGATTGACTGGGTGGATAGATGAA |
| 647 | TTTCATCTATCCACCCAGTCAATCA | 1911 | TGATTGACTGGGTGGATAGATGAAA |
| 648 | ATTTCATCTATCCACCCAGTCAATC | 1912 | GATTGACTGGGTGGATAGATGAAAT |
| 649 | CATTTCATCTATCCACCCAGTCAAT | 1913 | ATTGACTGGGTGGATAGATGAAATG |
| 650 | ACATTTCATCTATCCACCCAGTCAA | 1914 | TTGACTGGGTGGATAGATGAAATGT |
| 651 | AACATTTCATCTATCCACCCAGTCA | 1915 | TGACTGGGTGGATAGATGAAATGTT |
| 652 | CAACATTTCATCTATCCACCCAGTC | 1916 | GACTGGGTGGATAGATGAAATGTTG |
| 653 | CCAACATTTCATCTATCCACCCAGT | 1917 | ACTGGGTGGATAGATGAAATGTTGG |
| 654 | TCCAACATTTCATCTATCCACCCAG | 1918 | CTGGGTGGATAGATGAAATGTTGGA |
| 655 | ATCCAACATTTCATCTATCCACCCA | 1919 | TGGGTGGATAGATGAAATGTTGGAT |
| 656 | CATCCAACATTTCATCTATCCACCC | 1920 | GGGTGGATAGATGAAATGTTGGATG |
| 657 | TCATCCAACATTTCATCTATCCACC | 1921 | GGTGGATAGATGAAATGTTGGATGA |
| 658 | CTCATCCAACATTTCATCTATCCAC | 1922 | GTGGATAGATGAAATGTTGGATGAG |
| 659 | GCTCATCCAACATTTCATCTATCCA | 1923 | TGGATAGATGAAATGTTGGATGAGC |
| 660 | TGCTCATCCAACATTTCATCTATCC | 1924 | GGATAGATGAAATGTTGGATGAGCA |
| 661 | CTGCTCATCCAACATTTCATCTATC | 1925 | GATAGATGAAATGTTGGATGAGCAG |
| 662 | TCTGCTCATCCAACATTTCATCTAT | 1926 | ATAGATGAAATGTTGGATGAGCAGA |
| 663 | ATCTGCTCATCCAACATTTCATCTA | 1927 | TAGATGAAATGTTGGATGAGCAGAT |
| 664 | AATCTGCTCATCCAACATTTCATCT | 1928 | AGATGAAATGTTGGATGAGCAGATT |
| 665 | TAATCTGCTCATCCAACATTTCATC | 1929 | GATGAAATGTTGGATGAGCAGATTA |
| 666 | TTAATCTGCTCATCCAACATTTCAT | 1930 | ATGAAATGTTGGATGAGCAGATTAA |
| 667 | CTTAATCTGCTCATCCAACATTTCA | 1931 | TGAAATGTTGGATGAGCAGATTAAG |
| 668 | ACTTAATCTGCTCATCCAACATTTC | 1932 | GAAATGTTGGATGAGCAGATTAAGT |
| 669 | AACTTAATCTGCTCATCCAACATTT | 1933 | AAATGTTGGATGAGCAGATTAAGTT |
| 670 | CAACTTAATCTGCTCATCCAACATT | 1934 | AATGTTGGATGAGCAGATTAAGTTG |
| 671 | ACAACTTAATCTGCTCATCCAACAT | 1935 | ATGTTGGATGAGCAGATTAAGTTGT |
| 672 | TACAACTTAATCTGCTCATCCAACA | 1936 | TGTTGGATGAGCAGATTAAGTTGTA |
| 673 | ATACAACTTAATCTGCTCATCCAAC | 1937 | GTTGGATGAGCAGATTAAGTTGTAT |
| 674 | AATACAACTTAATCTGCTCATCCAA | 1938 | TTGGATGAGCAGATTAAGTIGTATT |
| 675 | CAATACAACTTAATCTGCTCATCCA | 1939 | TGGATGAGCAGATTAAGTTGTATTG |
| 676 | CCAATACAACTTAATCTGCTCATCC | 1940 | GGATGAGCAGATTAAGTTGTATTGG |
| 677 | TCCAATACAACTTAATCTGCTCATC | 1941 | GATGAGCAGATTAAGTTGTATTGGA |
| 678 | ATCCAATACAACTTAATCTGCTCAT | 1942 | ATGAGCAGATTAAGTTGTATTGGAT |
| 679 | CATCCAATACAACTTAATCTGCTCA | 1943 | TGAGCAGATTAAGTTGTATTGGATG |
| 680 | CCATCCAATACAACTTAATCTGCTC | 1944 | GAGCAGATTAAGTTGTATTGGATGG |
| 681 | CCCATCCAATACAACTTAATCTGCT | 1945 | AGCAGATTAAGTTGTATTGGATGGG |
| 682 | TCCCATCCAATACAACTTAATCTGC | 1946 | GCAGATTAAGTTGTATTGGATGGGA |
| 683 | ATCCCATCCAATACAACTTAATCTG | 1947 | CAGATTAAGTTGTATTGGATGGGAT |
| 684 | CATCCCATCCAATACAACTTAATCT | 1948 | AGATTAAGTTGTATTGGATGGGATG |
| 685 | CCATCCCATCCAATACAACTTAATC | 1949 | GATTAAGTTGTATTGGATGGGATGG |
| 686 | TCCATCCCATCCAATACAACTTAAT | 1950 | ATTAAGTTGTATTGGATGGGATGGA |
| 687 | ATCCATCCCATCCAATACAACTTAA | 1951 | TTAAGTTGTATTGGATGGGATGGAT |
| 688 | CATCCATCCCATCCAATACAACTTA | 1952 | TAAGTTGTATTGGATGGGATGGATG |
| 689 | CCATCCATCCCATCCAATACAACTT | 1953 | AAGTTGTATTGGATGGGATGGATGG |
| 690 | TCCATCCATCCCATCCAATACAACT | 1954 | AGTTGTATTGGATGGGATGGATGGA |
| 691 | TTCCATCCATCCCATCCAATACAAC | 1955 | GTTGTATTGGATGGGATGGATGGAA |
| 692 | CTTCCATCCATCCCATCCAATACAA | 1956 | TTGTATTGGATGGGATGGATGGAAG |
| 693 | ACTTCCATCCATCCCATCCAATACA | 1957 | TGTATTGGATGGGATGGATGGAAGT |
| 694 | CACTTCCATCCATCCCATCCAATAC | 1958 | GTATTGGATGGGATGGATGGAAGTG |
| 695 | ACACTTCCATCCATCCCATCCAATA | 1959 | TATTGGATGGGATGGATGGAAGTGT |
| 696 | CACACTTCCATCCATCCCATCCAAT | 1960 | ATTGGATGGGATGGATGGAAGTGTG |
| 697 | CCACACTTCCATCCATCCCATCCAA | 1961 | TTGGATGGGATGGATGGAAGTGTGG |
| 698 | ACCACACTTCCATCCATCCCATCCA | 1962 | TGGATGGGATGGATGGAAGTGTGGT |
| 699 | AACCACACTTCCATCCATCCCATCC | 1963 | GGATGGGATGGATGGAAGTGTGGTT |
| 700 | CAACCACACTTCCATCCATCCCATC | 1964 | GATGGGATGGATGGAAGTGTGGTTG |
| 701 | TCAACCACACTTCCATCCATCCCAT | 1965 | ATGGGATGGATGGAAGTGTGGTTGA |
| 702 | CTCAACCACACTTCCATCCATCCCA | 1966 | TGGGATGGATGGAAGTGTGGTTGAG |
| 703 | ACTCAACCACACTTCCATCCATCCC | 1967 | GGGATGGATGGAAGTGTGGTTGAGT |
| 704 | AACTCAACCACACTTCCATCCATCC | 1968 | GGATGGATGGAAGTGTGGTTGAGTT |
| 705 | TAACTCAACCACACTTCCATCCATC | 1969 | GATGGATGGAAGTGTGGTTGAGTTA |
| 706 | ATAACTCAACCACACTTCCATCCAT | 1970 | ATGGATGGAAGTGTGGTTGAGTTAT |
| 707 | AATAACTCAACCACACTTCCATCCA | 1971 | TGGATGGAAGTGTGGTTGAGTTATT |
| 708 | TAATAACTCAACCACACTTCCATCC | 1972 | GGATGGAAGTGTGGTTGAGTTATTA |
| 709 | CTAATAACTCAACCACACTTCCATC | 1973 | GATGGAAGTGTGGTTGAGTTATTAG |
| 710 | TCTAATAACTCAACCACACTTCCAT | 1974 | ATGGAAGTGTGGTTGAGTTATTAGA |
| 711 | TTCTAATAACTCAACCACACTTCCA | 1975 | TGGAAGTGTGGTTGAGTTATTAGAA |
| 712 | CTTCTAATAACTCAACCACACTTCC | 1976 | GGAAGTGTGGTTGAGTTATTAGAAG |
| 713 | CCTTCTAATAACTCAACCACACTTC | 1977 | GAAGTGTGGTTGAGTTATTAGAAGG |
| 714 | TCCTTCTAATAACTCAACCACACTT | 1978 | AAGTGTGGTTGAGTTATTAGAAGGA |
| 715 | TTCCTTCTAATAACTCAACCACACT | 1979 | AGTGTGGTTGAGTTATTAGAAGGAA |
| 716 | CTTCCTTCTAATAACTCAACCACAC | 1980 | GTGTGGTTGAGTTATTAGAAGGAAG |
| 717 | TCTTCCTTCTAATAACTCAACCACA | 1981 | TGTGGTTGAGTTATTAGAAGGAAGA |
| 718 | ATCTTCCTTCTAATAACTCAACCAC | 1982 | GTGGTTGAGTTATTAGAAGGAAGAT |
| 719 | AATCTTCCTTCTAATAACTCAACCA | 1983 | TGGTTGAGTTATTAGAAGGAAGATT |
| 720 | CAATCTTCCTTCTAATAACTCAACC | 1984 | GGTTGAGTTATTAGAAGGAAGATTG |
| 721 | TCAATCTTCCTTCTAATAACTCAAC | 1985 | GTTGAGTTATTAGAAGGAAGATTGA |
| 722 | CTCAATCTTCCTTCTAATAACTCAA | 1986 | TTGAGTTATTAGAAGGAAGATTGAG |
| 723 | ACTCAATCTTCCTTCTAATAACTCA | 1987 | TGAGTTATTAGAAGGAAGATTGAGT |
| 724 | TACTCAATCTTCCTTCTAATAACTC | 1988 | GAGTTATTAGAAGGAAGATTGAGTA |
| 725 | CTACTCAATCTTCCTTCTAATAACT | 1989 | AGTTATTAGAAGGAAGATTGAGTAG |
| 726 | TCTACTCAATCTTCCTTCTAATAAC | 1990 | GTTATTAGAAGGAAGATTGAGTAGA |
| 727 | ATCTACTCAATCTTCCTTCTAATAA | 1991 | TTATTAGAAGGAAGATTGAGTAGAT |
| 728 | TATCTACTCAATCTTCCTTCTAATA | 1992 | TATTAGAAGGAAGATTGAGTAGATA |
| 729 | CTATCTACTCAATCTTCCTTCTAAT | 1993 | ATTAGAAGGAAGATTGAGTAGATAG |
| 730 | CCTATCTACTCAATCTTCCTTCTAA | 1994 | TTAGAAGGAAGATTGAGTAGATAGG |
| 731 | ACCTATCTACTCAATCTTCCTTCTA | 1995 | TAGAAGGAAGATTGAGTAGATAGGT |
| 732 | CACCTATCTACTCAATCTTCCTTCT | 1996 | AGAAGGAAGATTGAGTAGATAGGTG |
| 733 | TCACCTATCTACTCAATCTTCCTTC | 1997 | GAAGGAAGATTGAGTAGATAGGTGA |
| 734 | TTCACCTATCTACTCAATCTTCCTT | 1998 | AAGGAAGATTGAGTAGATAGGTGAA |
| 735 | ATTCACCTATCTACTCAATCTTCCT | 1999 | AGGAAGATTGAGTAGATAGGTGAAT |
| 736 | AATTCACCTATCTACTCAATCTTCC | 2000 | GGAAGATTGAGTAGATAGGTGAATT |
| 737 | AAATTCACCTATCTACTCAATCTTC | 2001 | GAAGATTGAGTAGATAGGTGAATTT |
| 738 | CAAATTCACCTATCTACTCAATCTT | 2002 | AAGATTGAGTAGATAGGTGAATTTG |
| 739 | ACAAATTCACCTATCTACTCAATCT | 2003 | AGATTGAGTAGATAGGTGAATTTGT |
| 740 | AACAAATTCACCTATCTACTCAATC | 2004 | GATTGAGTAGATAGGTGAATTTGTT |
| 741 | CAACAAATTCACCTATCTACTCAAT | 2005 | ATTGAGTAGATAGGTGAATTTGTTG |
| 742 | TCAACAAATTCACCTATCTACTCAA | 2006 | TTGAGTAGATAGGTGAATTTGTTGA |
| 743 | ATCAACAAATTCACCTATCTACTCA | 2007 | TGAGTAGATAGGTGAATTTGTTGAT |
| 744 | TATCAACAAATTCACCTATCTACTC | 2008 | GAGTAGATAGGTGAATTTGTTGATA |
| 745 | CTATCAACAAATTCACCTATCTACT | 2009 | AGTAGATAGGTGAATTTGTTGATAG |
| 746 | ACTATCAACAAATTCACCTATCTAC | 2010 | GTAGATAGGTGAATTTGTTGATAGT |
| 747 | GACTATCAACAAATTCACCTATCTA | 2011 | TAGATAGGTGAATTTGTTGATAGTC |
| 748 | TGACTATCAACAAATTCACCTATCT | 2012 | AGATAGGTGAATTTGTTGATAGTCA |
| 749 | CTGACTATCAACAAATTCACCTATC | 2013 | GATAGGTGAATTTGTTGATAGTCAG |
| 750 | TCTGACTATCAACAAATTCACCTAT | 2014 | ATAGGTGAATTTGTTGATAGTCAGA |
| 751 | ATCTGACTATCAACAAATTCACCTA | 2015 | TAGGTGAATTTGTTGATAGTCAGAT |
| 752 | CATCTGACTATCAACAAATTCACCT | 2016 | AGGTGAATTTGTTGATAGTCAGATG |
| 753 | CCATCTGACTATCAACAAATTCACC | 2017 | GGTGAATTTGTTGATAGTCAGATGG |
| 754 | CCCATCTGACTATCAACAAATTCAC | 2018 | GTGAATTTGTTGATAGTCAGATGGG |
| 755 | ACCCATCTGACTATCAACAAATTCA | 2019 | TGAATTTGTTGATAGTCAGATGGGT |
| 756 | TACCCATCTGACTATCAACAAATTC | 2020 | GAATTTGTTGATAGTCAGATGGGTA |
| 757 | CTACCCATCTGACTATCAACAAATT | 2021 | AATTTGTTGATAGTCAGATGGGTAG |
| 758 | TCTACCCATCTGACTATCAACAAAT | 2022 | ATTTGTTGATAGTCAGATGGGTAGA |
| 759 | ATCTACCCATCTGACTATCAACAAA | 2023 | TTTGTTGATAGTCAGATGGGTAGAT |
| 760 | TATCTACCCATCTGACTATCAACAA | 2024 | TTGTTGATAGTCAGATGGGTAGATA |
| 761 | CTATCTACCCATCTGACTATCAACA | 2025 | TGTTGATAGTCAGATGGGTAGATAG |
| 762 | CCTATCTACCCATCTGACTATCAAC | 2026 | GTTGATAGTCAGATGGGTAGATAGG |
| 763 | ACCTATCTACCCATCTGACTATCAA | 2027 | TTGATAGTCAGATGGGTAGATAGGT |
| 764 | TACCTATCTACCCATCTGACTATCA | 2028 | TGATAGTCAGATGGGTAGATAGGTA |
| 765 | CTACCTATCTACCCATCTGACTATC | 2029 | GATAGTCAGATGGGTAGATAGGTAG |
| 766 | TCTACCTATCTACCCATCTGACTAT | 2030 | ATAGTCAGATGGGTAGATAGGTAGA |
| 767 | ATCTACCTATCTACCCATCTGACTA | 2031 | TAGTCAGATGGGTAGATAGGTAGAT |
| 768 | CATCTACCTATCTACCCATCTGACT | 2032 | AGTCAGATGGGTAGATAGGTAGATG |
| 769 | CCATCTACCTATCTACCCATCTGAC | 2033 | GTCAGATGGGTAGATAGGTAGATGG |
| 770 | TCCATCTACCTATCTACCCATCTGA | 2034 | TCAGATGGGTAGATAGGTAGATGGA |
| 771 | ATCCATCTACCTATCTACCCATCTG | 2035 | CAGATGGGTAGATAGGTAGATGGAT |
| 772 | CATCCATCTACCTATCTACCCATCT | 2036 | AGATGGGTAGATAGGTAGATGGATG |
| 773 | CCATCCATCTACCTATCTACCCATC | 2037 | GATGGGTAGATAGGTAGATGGATGG |
| 774 | TCCATCCATCTACCTATCTACCCAT | 2038 | ATGGGTAGATAGGTAGATGGATGGA |
| 775 | ATCCATCCATCTACCTATCTACCCA | 2039 | TGGGTAGATAGGTAGATGGATGGAT |
| 776 | CATCCATCCATCTACCTATCTACCC | 2040 | GGGTAGATAGGTAGATGGATGGATG |
| 777 | CCATCCATCCATCTACCTATCTACC | 2041 | GGTAGATAGGTAGATGGATGGATGG |
| 778 | TCCATCCATCCATCTACCTATCTAC | 2042 | GTAGATAGGTAGATGGATGGATGGA |
| 779 | ATCCATCCATCCATCTACCTATCTA | 2043 | TAGATAGGTAGATGGATGGATGGAT |
| 780 | CATCCATCCATCCATCTACCTATCT | 2044 | AGATAGGTAGATGGATGGATGGATG |
| 781 | CCATCCATCCATCCATCTACCTATC | 2045 | GATAGGTAGATGGATGGATGGATGG |
| 782 | TCCATCCATCCATCCATCTACCTAT | 2046 | ATAGGTAGATGGATGGATGGATGGA |
| 783 | ATCCATCCATCCATCCATCTACCTA | 2047 | TAGGTAGATGGATGGATGGATGGAT |
| 784 | CATCCATCCATCCATCCATCTACCT | 2048 | AGGTAGATGGATGGATGGATGGATG |
| 785 | CCATCCATCCATCCATCCATCTACC | 2049 | GGTAGATGGATGGATGGATGGATGG |
| 786 | TCCATCCATCCATCCATCCATCTAC | 2050 | GTAGATGGATGGATGGATGGATGGA |
| 787 | ATCCATCCATCCATCCATCCATCTA | 2051 | TAGATGGATGGATGGATGGATGGAT |
| 788 | CATCCATCCATCCATCCATCCATCT | 2052 | AGATGGATGGATGGATGGATGGATG |
| 789 | ACATCCATCCATCCATCCATCCATC | 2053 | GATGGATGGATGGATGGATGGATGT |
| 790 | TACATCCATCCATCCATCCATCCAT | 2054 | ATGGATGGATGGATGGATGGATGTA |
| 791 | ATACATCCATCCATCCATCCATCCA | 2055 | TGGATGGATGGATGGATGGATGTAT |
| 792 | TATACATCCATCCATCCATCCATCC | 2056 | GGATGGATGGATGGATGGATGTATA |
| 793 | CTATACATCCATCCATCCATCCATC | 2057 | GATGGATGGATGGATGGATGTATAG |
| 794 | CCTATACATCCATCCATCCATCCAT | 2058 | ATGGATGGATGGATGGATGTATAGG |
| 795 | GCCTATACATCCATCCATCCATCCA | 2059 | TGGATGGATGGATGGATGTATAGGC |
| 796 | TGCCTATACATCCATCCATCCATCC | 2060 | GGATGGATGGATGGATGTATAGGCA |
| 797 | CTGCCTATACATCCATCCATCCATC | 2061 | GATGGATGGATGGATGTATAGGCAG |
| 798 | TCTGCCTATACATCCATCCATCCAT | 2062 | ATGGATGGATGGATGTATAGGCAGA |
| 799 | ATCTGCCTATACATCCATCCATCCA | 2063 | TGGATGGATGGATGTATAGGCAGAT |
| 800 | CATCTGCCTATACATCCATCCATCC | 2064 | GGATGGATGGATGTATAGGCAGATG |
| 801 | CCATCTGCCTATACATCCATCCATC | 2065 | GATGGATGGATGTATAGGCAGATGG |
| 802 | TCCATCTGCCTATACATCCATCCAT | 2066 | ATGGATGGATGTATAGGCAGATGGA |
| 803 | GTCCATCTGCCTATACATCCATCCA | 2067 | TGGATGGATGTATAGGCAGATGGAC |
| 804 | TGTCCATCTGCCTATACATCCATCC | 2068 | GGATGGATGTATAGGCAGATGGACA |
| 805 | TTGTCCATCTGCCTATACATCCATC | 2069 | GATGGATGTATAGGCAGATGGACAA |
| 806 | TTTGTCCATCTGCCTATACATCCAT | 2070 | ATGGATGTATAGGCAGATGGACAAA |
| 807 | ATTTGTCCATCTGCCTATACATCCA | 2071 | TGGATGTATAGGCAGATGGACAAAT |
| 808 | CATTTGTCCATCTGCCTATACATCC | 2072 | GGATGTATAGGCAGATGGACAAATG |
| 809 | CCATTTGTCCATCTGCCTATACATC | 2073 | GATGTATAGGCAGATGGACAAATGG |
| 810 | TCCATTTGTCCATCTGCCTATACAT | 2074 | ATGTATAGGCAGATGGACAAATGGA |
| 811 | ATCCATTTGTCCATCTGCCTATACA | 2075 | TGTATAGGCAGATGGACAAATGGAT |
| 812 | CATCCATTTGTCCATCTGCCTATAC | 2076 | GTATAGGCAGATGGACAAATGGATG |
| 813 | TCATCCATTTGTCCATCTGCCTATA | 2077 | TATAGGCAGATGGACAAATGGATGA |
| 814 | TTCATCCATTTGTCCATCTGCCTAT | 2078 | ATAGGCAGATGGACAAATGGATGAA |
| 815 | ATTCATCCATTTGTCCATCTGCCTA | 2079 | TAGGCAGATGGACAAATGGATGAAT |
| 816 | CATTCATCCATTTGTCCATCTGCCT | 2080 | AGGCAGATGGACAAATGGATGAATG |
| 817 | CCATTCATCCATTTGTCCATCTGCC | 2081 | GGCAGATGGACAAATGGATGAATGG |
| 818 | CCCATTCATCCATTTGTCCATCTGC | 2082 | GCAGATGGACAAATGGATGAATGGG |
| 819 | ACCCATTCATCCATTTGTCCATCTG | 2083 | CAGATGGACAAATGGATGAATGGGT |
| 820 | CACCCATTCATCCATTTGTCCATCT | 2084 | AGATGGACAAATGGATGAATGGGTG |
| 821 | CCACCCATTCATCCATTTGTCCATC | 2085 | GATGGACAAATGGATGAATGGGTGG |
| 822 | CCCACCCATTCATCCATTTGTCCAT | 2086 | ATGGACAAATGGATGAATGGGTGGG |
| 823 | ACCCACCCATTCATCCATTTGTCCA | 2087 | TGGACAAATGGATGAATGGGTGGGT |
| 824 | CACCCACCCATTCATCCATTTGTCC | 2088 | GGACAAATGGATGAATGGGTGGGTG |
| 825 | CCACCCACCCATTCATCCATTTGTC | 2089 | GACAAATGGATGAATGGGTGGGTGG |
| 826 | TCCACCCACCCATTCATCCATTTGT | 2090 | ACAAATGGATGAATGGGTGGGTGGA |
| 827 | ATCCACCCACCCATTCATCCATTTG | 2091 | CAAATGGATGAATGGGTGGGTGGAT |
| 828 | CATCCACCCACCCATTCATCCATTT | 2092 | AAATGGATGAATGGGTGGGTGGATG |
| 829 | TCATCCACCCACCCATTCATCCATT | 2093 | AATGGATGAATGGGTGGGTGGATGA |
| 830 | TTCATCCACCCACCCATTCATCCAT | 2094 | ATGGATGAATGGGTGGGTGGATGAA |
| 831 | ATTCATCCACCCACCCATTCATCCA | 2095 | TGGATGAATGGGTGGGTGGATGAAT |
| 832 | CATTCATCCACCCACCCATTCATCC | 2096 | GGATGAATGGGTGGGTGGATGAATG |
| 833 | CCATTCATCCACCCACCCATTCATC | 2097 | GATGAATGGGTGGGTGGATGAATGG |
| 834 | TCCATTCATCCACCCACCCATTCAT | 2098 | ATGAATGGGTGGGTGGATGAATGGA |
| 835 | TTCCATTCATCCACCCACCCATTCA | 2099 | TGAATGGGTGGGTGGATGAATGGAA |
| 836 | CTTCCATTCATCCACCCACCCATTC | 2100 | GAATGGGTGGGTGGATGAATGGAAG |
| 837 | CCTTCCATTCATCCACCCACCCATT | 2101 | AATGGGTGGGTGGATGAATGGAAGG |
| 838 | TCCTTCCATTCATCCACCCACCCAT | 2102 | ATGGGTGGGTGGATGAATGGAAGGA |
| 839 | ATCCTTCCATTCATCCACCCACCCA | 2103 | TGGGTGGGTGGATGAATGGAAGGAT |
| 840 | CATCCTTCCATTCATCCACCCACCC | 2104 | GGGTGGGTGGATGAATGGAAGGATG |
| 841 | ACATCCTTCCATTCATCCACCCACC | 2105 | GGTGGGTGGATGAATGGAAGGATGT |
| 842 | CACATCCTTCCATTCATCCACCCAC | 2106 | GTGGGTGGATGAATGGAAGGATGTG |
| 843 | ACACATCCTTCCATTCATCCACCCA | 2107 | TGGGTGGATGAATGGAAGGATGTGT |
| 844 | CACACATCCTTCCATTCATCCACCC | 2108 | GGGTGGATGAATGGAAGGATGTGTG |
| 845 | CCACACATCCTTCCATTCATCCACC | 2109 | GGTGGATGAATGGAAGGATGTGTGG |
| 846 | ACCACACATCCTTCCATTCATCCAC | 2110 | GTGGATGAATGGAAGGATGTGTGGT |
| 847 | AACCACACATCCTTCCATTCATCCA | 2111 | TGGATGAATGGAAGGATGTGTGGTT |
| 848 | CAACCACACATCCTTCCATTCATCC | 2112 | GGATGAATGGAAGGATGTGTGGTTG |
| 849 | TCAACCACACATCCTTCCATTCATC | 2113 | GATGAATGGAAGGATGTGTGGTTGA |
| 850 | TTCAACCACACATCCTTCCATTCAT | 2114 | ATGAATGGAAGGATGTGTGGTTGAA |
| 851 | GTTCAACCACACATCCTTCCATTCA | 2115 | TGAATGGAAGGATGTGTGGTTGAAC |
| 852 | AGTTCAACCACACATCCTTCCATTC | 2116 | GAATGGAAGGATGTGTGGTTGAACT |
| 853 | TAGTTCAACCACACATCCTTCCATT | 2117 | AATGGAAGGATGTGTGGTTGAACTA |
| 854 | ATAGTTCAACCACACATCCTTCCAT | 2118 | ATGGAAGGATGTGTGGTTGAACTAT |
| 855 | AATAGTTCAACCACACATCCTTCCA | 2119 | TGGAAGGATGTGTGGTTGAACTATT |
| 856 | CAATAGTTCAACCACACATCCTTCC | 2120 | GGAAGGATGTGTGGTTGAACTATTG |
| 857 | GCAATAGTTCAACCACACATCCTTC | 2121 | GAAGGATGTGTGGTTGAACTATTGC |
| 858 | TGCAATAGTTCAACCACACATCCTT | 2122 | AAGGATGTGTGGTTGAACTATTGCA |
| 859 | TTGCAATAGTTCAACCACACATCCT | 2123 | AGGATGTGTGGTTGAACTATTGCAA |
| 860 | CTTGCAATAGTTCAACCACACATCC | 2124 | GGATGTGTGGTTGAACTATTGCAAG |
| 861 | ACTTGCAATAGTTCAACCACACATC | 2125 | GATGTGTGGTTGAACTATTGCAAGT |
| 862 | TACTTGCAATAGTTCAACCACACAT | 2126 | ATGTGTGGTTGAACTATTGCAAGTA |
| 863 | ATACTTGCAATAGTTCAACCACACA | 2127 | TGTGTGGTTGAACTATTGCAAGTAT |
| 864 | AATACTTGCAATAGTTCAACCACAC | 2128 | GTGTGGTTGAACTATTGCAAGTATT |
| 865 | CAATACTTGCAATAGTTCAACCACA | 2129 | TGTGGTTGAACTATTGCAAGTATTG |
| 866 | TCAATACTTGCAATAGTTCAACCAC | 2130 | GTGGTTGAACTATTGCAAGTATTGA |
| 867 | ATCAATACTTGCAATAGTTCAACCA | 2131 | TGGTTGAACTATTGCAAGTATTGAT |
| 868 | TATCAATACTTGCAATAGTTCAACC | 2132 | GGTTGAACTATTGCAAGTATTGATA |
| 869 | TTATCAATACTTGCAATAGTTCAAC | 2133 | GTTGAACTATTGCAAGTATTGATAA |
| 870 | ATTATCAATACTTGCAATAGTTCAA | 2134 | TTGAACTATTGCAAGTATTGATAAT |
| 871 | AATTATCAATACTTGCAATAGTTCA | 2135 | TGAACTATTGCAAGTATTGATAATT |
| 872 | CAATTATCAATACTTGCAATAGTTC | 2136 | GAACTATTGCAAGTATTGATAATTG |
| 873 | CCAATTATCAATACTTGCAATAGTT | 2137 | AACTATTGCAAGTATTGATAATTGG |
| 874 | CCCAATTATCAATACTTGCAATAGT | 2138 | ACTATTGCAAGTATTGATAATTGGG |
| 875 | ACCCAATTATCAATACTTGCAATAG | 2139 | CTATTGCAAGTATTGATAATTGGGT |
| 876 | AACCCAATTATCAATACTTGCAATA | 2140 | TATTGCAAGTATTGATAATTGGGTT |
| 877 | GAACCCAATTATCAATACTTGCAAT | 2141 | ATTGCAAGTATTGATAATTGGGTTC |
| 878 | TGAACCCAATTATCAATACTTGCAA | 2142 | TTGCAAGTATTGATAATTGGGTTCA |
| 879 | ATGAACCCAATTATCAATACTTGCA | 2143 | TGCAAGTATTGATAATTGGGTTCAT |
| 880 | TATGAACCCAATTATCAATACTTGC | 2144 | GCAAGTATTGATAATTGGGTTCATA |
| 881 | TTATGAACCCAATTATCAATACTTG | 2145 | CAAGTATTGATAATTGGGTTCATAA |
| 882 | ATTATGAACCCAATTATCAATACTT | 2146 | AAGTATTGATAATTGGGTTCATAAT |
| 883 | AATTATGAACCCAATTATCAATACT | 2147 | AGTATTGATAATTGGGTTCATAATT |
| 884 | AAATTATGAACCCAATTATCAATAC | 2148 | GTATTGATAATTGGGTTCATAATTT |
| 885 | GAAATTATGAACCCAATTATCAATA | 2149 | TATTGATAATTGGGTTCATAATTTC |
| 886 | AGAAATTATGAACCCAATTATCAAT | 2150 | ATTGATAATTGGGTTCATAATTTCT |
| 887 | CAGAAATTATGAACCCAATTATCAA | 2151 | TTGATAATTGGGTTCATAATTTCTG |
| 888 | TCAGAAATTATGAACCCAATTATCA | 2152 | TGATAATTGGGTTCATAATTTCTGA |
| 889 | TTCAGAAATTATGAACCCAATTATC | 2153 | GATAATTGGGTTCATAATTTCTGAA |
| 890 | ATTCAGAAATTATGAACCCAATTAT | 2154 | ATAATTGGGTTCATAATTTCTGAAT |
| 891 | TATTCAGAAATTATGAACCCAATTA | 2155 | TAATTGGGTTCATAATTTCTGAATA |
| 892 | ATATTCAGAAATTATGAACCCAATT | 2156 | AATTGGGTTCATAATTTCTGAATAT |
| 893 | AATATTCAGAAATTATGAACCCAAT | 2157 | ATTGGGTTCATAATTTCTGAATATT |
| 894 | AAATATTCAGAAATTATGAACCCAA | 2158 | TTGGGTTCATAATTTCTGAATATTT |
| 895 | TAAATATTCAGAAATTATGAACCCA | 2159 | TGGGTTCATAATTTCTGAATATTTA |
| 896 | CTAAATATTCAGAAATTATGAACCC | 2160 | GGGTTCATAATTTCTGAATATTTAG |
| 897 | TCTAAATATTCAGAAATTATGAACC | 2161 | GGTTCATAATTTCTGAATATTTAGA |
| 898 | ATCTAAATATTCAGAAATTATGAAC | 2162 | GTTCATAATTTCTGAATATTTAGAT |
| 899 | CATCTAAATATTCAGAAATTATGAA | 2163 | TTCATAATTTCTGAATATTTAGATG |
| 900 | CCATCTAAATATTCAGAAATTATGA | 2164 | TCATAATTTCTGAATATTTAGATGG |
| 901 | TCCATCTAAATATTCAGAAATTATG | 2165 | CATAATTTCTGAATATTTAGATGGA |
| 902 | ATCCATCTAAATATTCAGAAATTAT | 2166 | ATAATTTCTGAATATTTAGATGGAT |
| 903 | CATCCATCTAAATATTCAGAAATTA | 2167 | TAATTTCTGAATATTTAGATGGATG |
| 904 | CCATCCATCTAAATATTCAGAAATT | 2168 | AATTTCTGAATATTTAGATGGATGG |
| 905 | ACCATCCATCTAAATATTCAGAAAT | 2169 | ATTTCTGAATATTTAGATGGATGGT |
| 906 | AACCATCCATCTAAATATTCAGAAA | 2170 | TTTCTGAATATTTAGATGGATGGTT |
| 907 | CAACCATCCATCTAAATATTCAGAA | 2171 | TTCTGAATATTTAGATGGATGGTTG |
| 908 | ACAACCATCCATCTAAATATTCAGA | 2172 | TCTGAATATTTAGATGGATGGTTGT |
| 909 | CACAACCATCCATCTAAATATTCAG | 2173 | CTGAATATTTAGATGGATGGTTGTG |
| 910 | TCACAACCATCCATCTAAATATTCA | 2174 | TGAATATTTAGATGGATGGTTGTGA |
| 911 | CTCACAACCATCCATCTAAATATTC | 2175 | GAATATTTAGATGGATGGTTGTGAG |
| 912 | ACTCACAACCATCCATCTAAATATT | 2176 | AATATTTAGATGGATGGTTGTGAGT |
| 913 | CACTCACAACCATCCATCTAAATAT | 2177 | ATATTTAGATGGATGGTTGTGAGTG |
| 914 | CCACTCACAACCATCCATCTAAATA | 2178 | TATTTAGATGGATGGTTGTGAGTGG |
| 915 | GCCACTCACAACCATCCATCTAAAT | 2179 | ATTTAGATGGATGGTTGTGAGTGGC |
| 916 | AGCCACTCACAACCATCCATCTAAA | 2180 | TTTAGATGGATGGTTGTGAGTGGCT |
| 917 | CAGCCACTCACAACCATCCATCTAA | 2181 | TTAGATGGATGGTTGTGAGTGGCTG |
| 918 | CCAGCCACTCACAACCATCCATCTA | 2182 | TAGATGGATGGTTGTGAGTGGCTGG |
| 919 | ACCAGCCACTCACAACCATCCATCT | 2183 | AGATGGATGGTTGTGAGTGGCTGGT |
| 920 | CACCAGCCACTCACAACCATCCATC | 2184 | GATGGATGGTTGTGAGTGGCTGGTG |
| 921 | CCACCAGCCACTCACAACCATCCAT | 2185 | ATGGATGGTTGTGAGTGGCTGGTGG |
| 922 | TCCACCAGCCACTCACAACCATCCA | 2186 | TGGATGGTTGTGAGTGGCTGGTGGA |
| 923 | GTCCACCAGCCACTCACAACCATCC | 2187 | GGATGGTTGTGAGTGGCTGGTGGAC |
| 924 | TGTCCACCAGCCACTCACAACCATC | 2188 | GATGGTTGTGAGTGGCTGGTGGACA |
| 925 | CTGTCCACCAGCCACTCACAACCAT | 2189 | ATGGTTGTGAGTGGCTGGTGGACAG |
| 926 | TCTGTCCACCAGCCACTCACAACCA | 2190 | TGGTTGTGAGTGGCTGGTGGACAGA |
| 927 | GTCTGTCCACCAGCCACTCACAACC | 2191 | GGTTGTGAGTGGCTGGTGGACAGAC |
| 928 | CGTCTGTCCACCAGCCACTCACAAC | 2192 | GTTGTGAGTGGCTGGTGGACAGACG |
| 929 | TCGTCTGTCCACCAGCCACTCACAA | 2193 | TTGTGAGTGGCTGGTGGACAGACGA |
| 930 | TTCGTCTGTCCACCAGCCACTCACA | 2194 | TGTGAGTGGCTGGTGGACAGACGAA |
| 931 | TTTCGTCTGTCCACCAGCCACTCAC | 2195 | GTGAGTGGCTGGTGGACAGACGAAA |
| 932 | TTTTCGTCTGTCCACCAGCCACTCA | 2196 | TGAGTGGCTGGTGGACAGACGAAAA |
| 933 | TTTTTCGTCTGTCCACCAGCCACTC | 2197 | GAGTGGCTGGTGGACAGACGAAAAA |
| 934 | ATTTTTCGTCTGTCCACCAGCCACT | 2198 | AGTGGCTGGTGGACAGACGAAAAAT |
| 935 | CATTTTTCGTCTGTCCACCAGCCAC | 2199 | GTGGCTGGTGGACAGACGAAAAATG |
| 936 | CCATTTTTCGTCTGTCCACCAGCCA | 2200 | TGGCTGGTGGACAGACGAAAAATGG |
| 937 | TCCATTTTTCGTCTGTCCACCAGCC | 2201 | GGCTGGTGGACAGACGAAAAATGGA |
| 938 | ATCCATTTTTCGTCTGTCCACCAGC | 2202 | GCTGGTGGACAGACGAAAAATGGAT |
| 939 | CATCCATTTTTCGTCTGTCCACCAG | 2203 | CTGGTGGACAGACGAAAAATGGATG |
| 940 | CCATCCATTTTTCGTCTGTCCACCA | 2204 | TGGTGGACAGACGAAAAATGGATGG |
| 941 | ACCATCCATTTTTCGTCTGTCCACC | 2205 | GGTGGACAGACGAAAAATGGATGGT |
| 942 | AACCATCCATTTTTCGTCTGTCCAC | 2206 | GTGGACAGACGAAAAATGGATGGTT |
| 943 | CAACCATCCATTTTTCGTCTGTCCA | 2207 | TGGACAGACGAAAAATGGATGGTTG |
| 944 | CCAACCATCCATTTTTCGTCTGTCC | 2208 | GGACAGACGAAAAATGGATGGTTGG |
| 945 | TCCAACCATCCATTTTTCGTCTGTC | 2209 | GACAGACGAAAAATGGATGGTTGGA |
| 946 | ATCCAACCATCCATTTTTCGTCTGT | 2210 | ACAGACGAAAAATGGATGGTTGGAT |
| 947 | TATCCAACCATCCATTTTTCGTCTG | 2211 | CAGACGAAAAATGGATGGTTGGATA |
| 948 | TTATCCAACCATCCATTTTTCGTCT | 2212 | AGACGAAAAATGGATGGTTGGATAA |
| 949 | TTTATCCAACCATCCATTTTTCGTC | 2213 | GACGAAAAATGGATGGTTGGATAAA |
| 950 | ATTTATCCAACCATCCATTTTTCGT | 2214 | ACGAAAAATGGATGGTTGGATAAAT |
| 951 | AATTTATCCAACCATCCATTTTTCG | 2215 | CGAAAAATGGATGGTTGGATAAATT |
| 952 | CAATTTATCCAACCATCCATTTTTC | 2216 | GAAAAATGGATGGTTGGATAAATTG |
| 953 | TCAATTTATCCAACCATCCATTTTT | 2217 | AAAAATGGATGGTTGGATAAATTGA |
| 954 | ATCAATTTATCCAACCATCCATTTT | 2218 | AAAATGGATGGTTGGATAAATTGAT |
| 955 | CATCAATTTATCCAACCATCCATTT | 2219 | AAATGGATGGTTGGATAAATTGATG |
| 956 | CCATCAATTTATCCAACCATCCATT | 2220 | AATGGATGGTTGGATAAATTGATGG |
| 957 | CCCATCAATTTATCCAACCATCCAT | 2221 | ATGGATGGTTGGATAAATTGATGGG |
| 958 | ACCCATCAATTTATCCAACCATCCA | 2222 | TGGATGGTTGGATAAATTGATGGGT |
| 959 | CACCCATCAATTTATCCAACCATCC | 2223 | GGATGGTTGGATAAATTGATGGGTG |
| 960 | CCACCCATCAATTTATCCAACCATC | 2224 | GATGGTTGGATAAATTGATGGGTGG |
| 961 | TCCACCCATCAATTTATCCAACCAT | 2225 | ATGGTTGGATAAATTGATGGGTGGA |
| 962 | ATCCACCCATCAATTTATCCAACCA | 2226 | TGGTTGGATAAATTGATGGGTGGAT |
| 963 | CATCCACCCATCAATTTATCCAACC | 2227 | GGTTGGATAAATTGATGGGTGGATG |
| 964 | CCATCCACCCATCAATTTATCCAAC | 2228 | GTTGGATAAATTGATGGGTGGATGG |
| 965 | TCCATCCACCCATCAATTTATCCAA | 2229 | TTGGATAAATTGATGGGTGGATGGA |
| 966 | ATCCATCCACCCATCAATTTATCCA | 2230 | TGGATAAATTGATGGGTGGATGGAT |
| 967 | CATCCATCCACCCATCAATTTATCC | 2231 | GGATAAATTGATGGGTGGATGGATG |
| 968 | CCATCCATCCACCCATCAATTTATC | 2232 | GATAAATTGATGGGTGGATGGATGG |
| 969 | ACCATCCATCCACCCATCAATTTAT | 2233 | ATAAATTGATGGGTGGATGGATGGT |
| 970 | AACCATCCATCCACCCATCAATTTA | 2234 | TAAATTGATGGGTGGATGGATGGTT |
| 971 | CAACCATCCATCCACCCATCAATTT | 2235 | AAATTGATGGGTGGATGGATGGTTG |
| 972 | CCAACCATCCATCCACCCATCAATT | 2236 | AATTGATGGGTGGATGGATGGTTGG |
| 973 | ACCAACCATCCATCCACCCATCAAT | 2237 | ATTGATGGGTGGATGGATGGTTGGT |
| 974 | AACCAACCATCCATCCACCCATCAA | 2238 | TTGATGGGTGGATGGATGGTTGGTT |
| 975 | CAACCAACCATCCATCCACCCATCA | 2239 | TGATGGGTGGATGGATGGTTGGTTG |
| 976 | ACAACCAACCATCCATCCACCCATC | 2240 | GATGGGTGGATGGATGGTTGGTTGT |
| 977 | TACAACCAACCATCCATCCACCCAT | 2241 | ATGGGTGGATGGATGGTTGGTTGTA |
| 978 | ATACAACCAACCATCCATCCACCCA | 2242 | TGGGTGGATGGATGGTTGGTTGTAT |
| 979 | CATACAACCAACCATCCATCCACCC | 2243 | GGGTGGATGGATGGTTGGTTGTATG |
| 980 | TCATACAACCAACCATCCATCCACC | 2244 | GGTGGATGGATGGTTGGTTGTATGA |
| 981 | TTCATACAACCAACCATCCATCCAC | 2245 | GTGGATGGATGGTTGGTTGTATGAA |
| 982 | TTTCATACAACCAACCATCCATCCA | 2246 | TGGATGGATGGTTGGTTGTATGAAA |
| 983 | CTTTCATACAACCAACCATCCATCC | 2247 | GGATGGATGGTTGGTTGTATGAAAG |
| 984 | TCTTTCATACAACCAACCATCCATC | 2248 | GATGGATGGTTGGTTGTATGAAAGA |
| 985 | TTCTTTCATACAACCAACCATCCAT | 2249 | ATGGATGGTTGGTTGTATGAAAGAA |
| 986 | ATTCTTTCATACAACCAACCATCCA | 2250 | TGGATGGTTGGTTGTATGAAAGAAT |
| 987 | CATTCTTTCATACAACCAACCATCC | 2251 | GGATGGTTGGTTGTATGAAAGAATG |
| 988 | TCATTCTTTCATACAACCAACCATC | 2252 | GATGGTTGGTTGTATGAAAGAATGA |
| 989 | TTCATTCTTTCATACAACCAACCAT | 2253 | ATGGTTGGTTGTATGAAAGAATGAA |
| 990 | ATTCATTCTTTCATACAACCAACCA | 2254 | TGGTTGGTTGTATGAAAGAATGAAT |
| 991 | CATTCATTCTTTCATACAACCAACC | 2255 | GGTTGGTTGTATGAAAGAATGAATG |
| 992 | TCATTCATTCTTTCATACAACCAAC | 2256 | GTTGGTTGTATGAAAGAATGAATGA |
| 993 | ATCATTCATTCTTTCATACAACCAA | 2257 | TTGGTTGTATGAAAGAATGAATGAT |
| 994 | AATCATTCATTCTTTCATACAACCA | 2258 | TGGTTGTATGAAAGAATGAATGATT |
| 995 | CAATCATTCATTCTTTCATACAACC | 2259 | GGTTGTATGAAAGAATGAATGATTG |
| 996 | CCAATCATTCATTCTTTCATACAAC | 2260 | GTTGTATGAAAGAATGAATGATTGG |
| 997 | CCCAATCATTCATTCTTTCATACAA | 2261 | TTGTATGAAAGAATGAATGATTGGG |
| 998 | ACCCAATCATTCATTCTTTCATACA | 2262 | TGTATGAAAGAATGAATGATTGGGT |
| 999 | TACCCAATCATTCATTCTTTCATAC | 2263 | GTATGAAAGAATGAATGATTGGGTA |
| 1000 | CTACCCAATCATTCATTCTTTCATA | 2264 | TATGAAAGAATGAATGATTGGGTAG |
| 1001 | CCTACCCAATCATTCATTCTTTCAT | 2265 | ATGAAAGAATGAATGATTGGGTAGG |
| 1002 | ACCTACCCAATCATTCATTCTTTCA | 2266 | TGAAAGAATGAATGATTGGGTAGGT |
| 1003 | CACCTACCCAATCATTCATTCTTTC | 2267 | GAAAGAATGAATGATTGGGTAGGTG |
| 1004 | CCACCTACCCAATCATTCATTCTTT | 2268 | AAAGAATGAATGATTGGGTAGGTGG |
| 1005 | TCCACCTACCCAATCATTCATTCTT | 2269 | AAGAATGAATGATTGGGTAGGTGGA |
| 1006 | ATCCACCTACCCAATCATTCATTCT | 2270 | AGAATGAATGATTGGGTAGGTGGAT |
| 1007 | AATCCACCTACCCAATCATTCATTC | 2271 | GAATGAATGATTGGGTAGGTGGATT |
| 1008 | TAATCCACCTACCCAATCATTCATT | 2272 | AATGAATGATTGGGTAGGTGGATTA |
| 1009 | TTAATCCACCTACCCAATCATTCAT | 2273 | ATGAATGATTGGGTAGGTGGATTAA |
| 1010 | CTTAATCCACCTACCCAATCATTCA | 2274 | TGAATGATTGGGTAGGTGGATTAAG |
| 1011 | ACTTAATCCACCTACCCAATCATTC | 2275 | GAATGATTGGGTAGGTGGATTAAGT |
| 1012 | AACTTAATCCACCTACCCAATCATT | 2276 | AATGATTGGGTAGGTGGATTAAGTT |
| 1013 | CAACTTAATCCACCTACCCAATCAT | 2277 | ATGATTGGGTAGGTGGATTAAGTTG |
| 1014 | GCAACTTAATCCACCTACCCAATCA | 2278 | TGATTGGGTAGGTGGATTAAGTTGC |
| 1015 | CGCAACTTAATCCACCTACCCAATC | 2279 | GATTGGGTAGGTGGATTAAGTTGCG |
| 1016 | CCGCAACTTAATCCACCTACCCAAT | 2280 | ATTGGGTAGGTGGATTAAGTTGCGG |
| 1017 | TCCGCAACTTAATCCACCTACCCAA | 2281 | TTGGGTAGGTGGATTAAGTTGCGGA |
| 1018 | ATCCGCAACTTAATCCACCTACCCA | 2282 | TGGGTAGGTGGATTAAGTTGCGGAT |
| 1019 | GATCCGCAACTTAATCCACCTACCC | 2283 | GGGTAGGTGGATTAAGTTGCGGATC |
| 1020 | TGATCCGCAACTTAATCCACCTACC | 2284 | GGTAGGTGGATTAAGTTGCGGATCA |
| 1021 | TTGATCCGCAACTTAATCCACCTAC | 2285 | GTAGGTGGATTAAGTTGCGGATCAA |
| 1022 | ATTGATCCGCAACTTAATCCACCTA | 2286 | TAGGTGGATTAAGTTGCGGATCAAT |
| 1023 | CATTGATCCGCAACTTAATCCACCT | 2287 | AGGTGGATTAAGTTGCGGATCAATG |
| 1024 | ACATTGATCCGCAACTTAATCCACC | 2288 | GGTGGATTAAGTTGCGGATCAATGT |
| 1025 | TACATTGATCCGCAACTTAATCCAC | 2289 | GTGGATTAAGTTGCGGATCAATGTA |
| 1026 | ATACATTGATCCGCAACTTAATCCA | 2290 | TGGATTAAGTTGCGGATCAATGTAT |
| 1027 | CATACATTGATCCGCAACTTAATCC | 2291 | GGATTAAGTTGCGGATCAATGTATG |
| 1028 | CCATACATTGATCCGCAACTTAATC | 2292 | GATTAAGTTGCGGATCAATGTATGG |
| 1029 | CCCATACATTGATCCGCAACTTAAT | 2293 | ATTAAGTTGCGGATCAATGTATGGG |
| 1030 | TCCCATACATTGATCCGCAACTTAA | 2294 | TTAAGTTGCGGATCAATGTATGGGA |
| 1031 | ATCCCATACATTGATCCGCAACTTA | 2295 | TAAGTTGCGGATCAATGTATGGGAT |
| 1032 | CATCCCATACATTGATCCGCAACTT | 2296 | AAGTTGCGGATCAATGTATGGGATG |
| 1033 | CCATCCCATACATTGATCCGCAACT | 2297 | AGTTGCGGATCAATGTATGGGATGG |
| 1034 | TCCATCCCATACATTGATCCGCAAC | 2298 | GTTGCGGATCAATGTATGGGATGGA |
| 1035 | ATCCATCCCATACATTGATCCGCAA | 2299 | TTGCGGATCAATGTATGGGATGGAT |
| 1036 | CATCCATCCCATACATTGATCCGCA | 2300 | TGCGGATCAATGTATGGGATGGATG |
| 1037 | TCATCCATCCCATACATTGATCCGC | 2301 | GCGGATCAATGTATGGGATGGATGA |
| 1038 | TTCATCCATCCCATACATTGATCCG | 2302 | CGGATCAATGTATGGGATGGATGAA |
| 1039 | ATTCATCCATCCCATACATTGATCC | 2303 | GGATCAATGTATGGGATGGATGAAT |
| 1040 | CATTCATCCATCCCATACATTGATC | 2304 | GATCAATGTATGGGATGGATGAATG |
| 1041 | CCATTCATCCATCCCATACATTGAT | 2305 | ATCAATGTATGGGATGGATGAATGG |
| 1042 | TCCATTCATCCATCCCATACATTGA | 2306 | TCAATGTATGGGATGGATGAATGGA |
| 1043 | ATCCATTCATCCATCCCATACATTG | 2307 | CAATGTATGGGATGGATGAATGGAT |
| 1044 | CATCCATTCATCCATCCCATACATT | 2308 | AATGTATGGGATGGATGAATGGATG |
| 1045 | CCATCCATTCATCCATCCCATACAT | 2309 | ATGTATGGGATGGATGAATGGATGG |
| 1046 | TCCATCCATTCATCCATCCCATACA | 2310 | TGTATGGGATGGATGAATGGATGGA |
| 1047 | ATCCATCCATTCATCCATCCCATAC | 2311 | GTATGGGATGGATGAATGGATGGAT |
| 1048 | CATCCATCCATTCATCCATCCCATA | 2312 | TATGGGATGGATGAATGGATGGATG |
| 1049 | CCATCCATCCATTCATCCATCCCAT | 2313 | ATGGGATGGATGAATGGATGGATGG |
| 1050 | TCCATCCATCCATTCATCCATCCCA | 2314 | TGGGATGGATGAATGGATGGATGGA |
| 1051 | ATCCATCCATCCATTCATCCATCCC | 2315 | GGGATGGATGAATGGATGGATGGAT |
| 1052 | CATCCATCCATCCATTCATCCATCC | 2316 | GGATGGATGAATGGATGGATGGATG |
| 1053 | CCATCCATCCATCCATTCATCCATC | 2317 | GATGGATGAATGGATGGATGGATGG |
| 1054 | TCCATCCATCCATCCATTCATCCAT | 2318 | ATGGATGAATGGATGGATGGATGGA |
| 1055 | ATCCATCCATCCATCCATTCATCCA | 2319 | TGGATGAATGGATGGATGGATGGAT |
| 1056 | CATCCATCCATCCATCCATTCATCC | 2320 | GGATGAATGGATGGATGGATGGATG |
| 1057 | ACATCCATCCATCCATCCATTCATC | 2321 | GATGAATGGATGGATGGATGGATGT |
| 1058 | CACATCCATCCATCCATCCATTCAT | 2322 | ATGAATGGATGGATGGATGGATGTG |
| 1059 | ACACATCCATCCATCCATCCATTCA | 2323 | TGAATGGATGGATGGATGGATGTGT |
| 1060 | CACACATCCATCCATCCATCCATTC | 2324 | GAATGGATGGATGGATGGATGTGTG |
| 1061 | CCACACATCCATCCATCCATCCATT | 2325 | AATGGATGGATGGATGGATGTGTGG |
| 1062 | ACCACACATCCATCCATCCATCCAT | 2326 | ATGGATGGATGGATGGATGTGTGGT |
| 1063 | AACCACACATCCATCCATCCATCCA | 2327 | TGGATGGATGGATGGATGTGTGGTT |
| 1064 | CAACCACACATCCATCCATCCATCC | 2328 | GGATGGATGGATGGATGTGTGGTTG |
| 1065 | TCAACCACACATCCATCCATCCATC | 2329 | GATGGATGGATGGATGTGTGGTTGA |
| 1066 | TTCAACCACACATCCATCCATCCAT | 2330 | ATGGATGGATGGATGTGTGGTTGAA |
| 1067 | ATTCAACCACACATCCATCCATCCA | 2331 | TGGATGGATGGATGTGTGGTTGAAT |
| 1068 | AATTCAACCACACATCCATCCATCC | 2332 | GGATGGATGGATGTGTGGTTGAATT |
| 1069 | TAATTCAACCACACATCCATCCATC | 2333 | GATGGATGGATGTGTGGTTGAATTA |
| 1070 | GTAATTCAACCACACATCCATCCAT | 2334 | ATGGATGGATGTGTGGTTGAATTAC |
| 1071 | AGTAATTCAACCACACATCCATCCA | 2335 | TGGATGGATGTGTGGTTGAATTACT |
| 1072 | CAGTAATTCAACCACACATCCATCC | 2336 | GGATGGATGTGTGGTTGAATTACTG |
| 1073 | TCAGTAATTCAACCACACATCCATC | 2337 | GATGGATGTGTGGTTGAATTACTGA |
| 1074 | TTCAGTAATTCAACCACACATCCAT | 2338 | ATGGATGTGTGGTTGAATTACTGAA |
| 1075 | TTTCAGTAATTCAACCACACATCCA | 2339 | TGGATGTGTGGTTGAATTACTGAAA |
| 1076 | CTTTCAGTAATTCAACCACACATCC | 2340 | GGATGTGTGGTTGAATTACTGAAAG |
| 1077 | CCTTTCAGTAATTCAACCACACATC | 2341 | GATGTGTGGTTGAATTACTGAAAGG |
| 1078 | ACCTTTCAGTAATTCAACCACACAT | 2342 | ATGTGTGGTTGAATTACTGAAAGGT |
| 1079 | AACCTTTCAGTAATTCAACCACACA | 2343 | TGTGTGGTTGAATTACTGAAAGGTT |
| 1080 | CAACCTTTCAGTAATTCAACCACAC | 2344 | GTGTGGTTGAATTACTGAAAGGTTG |
| 1081 | CCAACCTTTCAGTAATTCAACCACA | 2345 | TGTGGTTGAATTACTGAAAGGTTGG |
| 1082 | TCCAACCTTTCAGTAATTCAACCAC | 2346 | GTGGTTGAATTACTGAAAGGTTGGA |
| 1083 | TTCCAACCTTTCAGTAATTCAACCA | 2347 | TGGTTGAATTACTGAAAGGTTGGAA |
| 1084 | CTTCCAACCTTTCAGTAATTCAACC | 2348 | GGTTGAATTACTGAAAGGTTGGAAG |
| 1085 | TCTTCCAACCTTTCAGTAATTCAAC | 2349 | GTTGAATTACTGAAAGGTTGGAAGA |
| 1086 | CTCTTCCAACCTTTCAGTAATTCAA | 2350 | TTGAATTACTGAAAGGTTGGAAGAG |
| 1087 | ACTCTTCCAACCTTTCAGTAATTCA | 2351 | TGAATTACTGAAAGGTTGGAAGAGT |
| 1088 | CACTCTTCCAACCTTTCAGTAATTC | 2352 | GAATTACTGAAAGGTTGGAAGAGTG |
| 1089 | CCACTCTTCCAACCTTTCAGTAATT | 2353 | AATTACTGAAAGGTTGGAAGAGTGG |
| 1090 | TCCACTCTTCCAACCTTTCAGTAAT | 2354 | ATTACTGAAAGGTTGGAAGAGTGGA |
| 1091 | ATCCACTCTTCCAACCTTTCAGTAA | 2355 | TTACTGAAAGGTTGGAAGAGTGGAT |
| 1092 | CATCCACTCTTCCAACCTTTCAGTA | 2356 | TACTGAAAGGTTGGAAGAGTGGATG |
| 1093 | CCATCCACTCTTCCAACCTTTCAGT | 2357 | ACTGAAAGGTTGGAAGAGTGGATGG |
| 1094 | CCCATCCACTCTTCCAACCTTTCAG | 2358 | CTGAAAGGTTGGAAGAGTGGATGGG |
| 1095 | ACCCATCCACTCTTCCAACCTTTCA | 2359 | TGAAAGGTTGGAAGAGTGGATGGGT |
| 1096 | CACCCATCCACTCTTCCAACCTTTC | 2360 | GAAAGGTTGGAAGAGTGGATGGGTG |
| 1097 | TCACCCATCCACTCTTCCAACCTTT | 2361 | AAAGGTTGGAAGAGTGGATGGGTGA |
| 1098 | TTCACCCATCCACTCTTCCAACCTT | 2362 | AAGGTTGGAAGAGTGGATGGGTGAA |
| 1099 | TTTCACCCATCCACTCTTCCAACCT | 2363 | AGGTTGGAAGAGTGGATGGGTGAAA |
| 1100 | ATTTCACCCATCCACTCTTCCAACC | 2364 | GGTTGGAAGAGTGGATGGGTGAAAT |
| 1101 | AATTTCACCCATCCACTCTTCCAAC | 2365 | GTTGGAAGAGTGGATGGGTGAAATT |
| 1102 | AAATTTCACCCATCCACTCTTCCAA | 2366 | TTGGAAGAGTGGATGGGTGAAATTT |
| 1103 | CAAATTTCACCCATCCACTCTTCCA | 2367 | TGGAAGAGTGGATGGGTGAAATTTG |
| 1104 | CCAAATTTCACCCATCCACTCTTCC | 2368 | GGAAGAGTGGATGGGTGAAATTTGG |
| 1105 | CCCAAATTTCACCCATCCACTCTTC | 2369 | GAAGAGTGGATGGGTGAAATTTGGG |
| 1106 | CCCCAAATTTCACCCATCCACTCTT | 2370 | AAGAGTGGATGGGTGAAATTTGGGG |
| 1107 | ACCCCAAATTTCACCCATCCACTCT | 2371 | AGAGTGGATGGGTGAAATTTGGGGT |
| 1108 | TACCCCAAATTTCACCCATCCACTC | 2372 | GAGTGGATGGGTGAAATTTGGGGTA |
| 1109 | CTACCCCAAATTTCACCCATCCACT | 2373 | AGTGGATGGGTGAAATTTGGGGTAG |
| 1110 | ACTACCCCAAATTTCACCCATCCAC | 2374 | GTGGATGGGTGAAATTTGGGGTAGT |
| 1111 | AACTACCCCAAATTTCACCCATCCA | 2375 | TGGATGGGTGAAATTTGGGGTAGTT |
| 1112 | TAACTACCCCAAATTTCACCCATCC | 2376 | GGATGGGTGAAATTTGGGGTAGTTA |
| 1113 | CTAACTACCCCAAATTTCACCCATC | 2377 | GATGGGTGAAATTTGGGGTAGTTAG |
| 1114 | TCTAACTACCCCAAATTTCACCCAT | 2378 | ATGGGTGAAATTTGGGGTAGTTAGA |
| 1115 | ATCTAACTACCCCAAATTTCACCCA | 2379 | TGGGTGAAATTTGGGGTAGTTAGAT |
| 1116 | CATCTAACTACCCCAAATTTCACCC | 2380 | GGGTGAAATTTGGGGTAGTTAGATG |
| 1117 | CCATCTAACTACCCCAAATTTCACC | 2381 | GGTGAAATTTGGGGTAGTTAGATGG |
| 1118 | CCCATCTAACTACCCCAAATTTCAC | 2382 | GTGAAATTTGGGGTAGTTAGATGGG |
| 1119 | ACCCATCTAACTACCCCAAATTTCA | 2383 | TGAAATTTGGGGTAGTTAGATGGGT |
| 1120 | CACCCATCTAACTACCCCAAATTTC | 2384 | GAAATTTGGGGTAGTTAGATGGGTG |
| 1121 | CCACCCATCTAACTACCCCAAATTT | 2385 | AAATTTGGGGTAGTTAGATGGGTGG |
| 1122 | CCCACCCATCTAACTACCCCAAATT | 2386 | AATTTGGGGTAGTTAGATGGGTGGG |
| 1123 | ACCCACCCATCTAACTACCCCAAAT | 2387 | ATTTGGGGTAGTTAGATGGGTGGGT |
| 1124 | CACCCACCCATCTAACTACCCCAAA | 2388 | TTTGGGGTAGTTAGATGGGTGGGTG |
| 1125 | ACACCCACCCATCTAACTACCCCAA | 2389 | TTGGGGTAGTTAGATGGGTGGGTGT |
| 1126 | CACACCCACCCATCTAACTACCCCA | 2390 | TGGGGTAGTTAGATGGGTGGGTGTG |
| 1127 | ACACACCCACCCATCTAACTACCCC | 2391 | GGGGTAGTTAGATGGGTGGGTGTGT |
| 1128 | CACACACCCACCCATCTAACTACCC | 2392 | GGGTAGTTAGATGGGTGGGTGTGTG |
| 1129 | CCACACACCCACCCATCTAACTACC | 2393 | GGTAGTTAGATGGGTGGGTGTGTGG |
| 1130 | TCCACACACCCACCCATCTAACTAC | 2394 | GTAGTTAGATGGGTGGGTGTGTGGA |
| 1131 | ATCCACACACCCACCCATCTAACTA | 2395 | TAGTTAGATGGGTGGGTGTGTGGAT |
| 1132 | CATCCACACACCCACCCATCTAACT | 2396 | AGTTAGATGGGTGGGTGTGTGGATG |
| 1133 | CCATCCACACACCCACCCATCTAAC | 2397 | GTTAGATGGGTGGGTGTGTGGATGG |
| 1134 | TCCATCCACACACCCACCCATCTAA | 2398 | TTAGATGGGTGGGTGTGTGGATGGA |
| 1135 | ATCCATCCACACACCCACCCATCTA | 2399 | TAGATGGGTGGGTGTGTGGATGGAT |
| 1136 | TATCCATCCACACACCCACCCATCT | 2400 | AGATGGGTGGGTGTGTGGATGGATA |
| 1137 | TTATCCATCCACACACCCACCCATC | 2401 | GATGGGTGGGTGTGTGGATGGATAA |
| 1138 | TTTATCCATCCACACACCCACCCAT | 2402 | ATGGGTGGGTGTGTGGATGGATAAA |
| 1139 | TTTTATCCATCCACACACCCACCCA | 2403 | TGGGTGGGTGTGTGGATGGATAAAA |
| 1140 | CTTTTATCCATCCACACACCCACCC | 2404 | GGGTGGGTGTGTGGATGGATAAAAG |
| 1141 | TCTTTTATCCATCCACACACCCACC | 2405 | GGTGGGTGTGTGGATGGATAAAAGA |
| 1142 | CTCTTTTATCCATCCACACACCCAC | 2406 | GTGGGTGTGTGGATGGATAAAAGAG |
| 1143 | ACTCTTTTATCCATCCACACACCCA | 2407 | TGGGTGTGTGGATGGATAAAAGAGT |
| 1144 | TACTCTTTTATCCATCCACACACCC | 2408 | GGGTGTGTGGATGGATAAAAGAGTA |
| 1145 | CTACTCTTTTATCCATCCACACACC | 2409 | GGTGTGTGGATGGATAAAAGAGTAG |
| 1146 | TCTACTCTTTTATCCATCCACACAC | 2410 | GTGTGTGGATGGATAAAAGAGTAGA |
| 1147 | ATCTACTCTTTTATCCATCCACACA | 2411 | TGTGTGGATGGATAAAAGAGTAGAT |
| 1148 | CATCTACTCTTTTATCCATCCACAC | 2412 | GTGTGGATGGATAAAAGAGTAGATG |
| 1149 | TCATCTACTCTTTTATCCATCCACA | 2413 | TGTGGATGGATAAAAGAGTAGATGA |
| 1150 | TTCATCTACTCTTTTATCCATCCAC | 2414 | GTGGATGGATAAAAGAGTAGATGAA |
| 1151 | ATTCATCTACTCTTTTATCCATCCA | 2415 | TGGATGGATAAAAGAGTAGATGAAT |
| 1152 | CATTCATCTACTCTTTTATCCATCC | 2416 | GGATGGATAAAAGAGTAGATGAATG |
| 1153 | TCATTCATCTACTCTTTTATCCATC | 2417 | GATGGATAAAAGAGTAGATGAATGA |
| 1154 | TTCATTCATCTACTCTTTTATCCAT | 2418 | ATGGATAAAAGAGTAGATGAATGAA |
| 1155 | ATTCATTCATCTACTCTTTTATCCA | 2419 | TGGATAAAAGAGTAGATGAATGAAT |
| 1156 | AATTCATTCATCTACTCTTTTATCC | 2420 | GGATAAAAGAGTAGATGAATGAATT |
| 1157 | TAATTCATTCATCTACTCTTTTATC | 2421 | GATAAAAGAGTAGATGAATGAATTA |
| 1158 | TTAATTCATTCATCTACTCTTTTAT | 2422 | ATAAAAGAGTAGATGAATGAATTAA |
| 1159 | ATTAATTCATTCATCTACTCTTTTA | 2423 | TAAAAGAGTAGATGAATGAATTAAT |
| 1160 | CATTAATTCATTCATCTACTCTTTT | 2424 | AAAAGAGTAGATGAATGAATTAATG |
| 1161 | TCATTAATTCATTCATCTACTCTTT | 2425 | AAAGAGTAGATGAATGAATTAATGA |
| 1162 | TTCATTAATTCATTCATCTACTCTT | 2426 | AAGAGTAGATGAATGAATTAATGAA |
| 1163 | ATTCATTAATTCATTCATCTACTCT | 2427 | AGAGTAGATGAATGAATTAATGAAT |
| 1164 | TATTCATTAATTCATTCATCTACTC | 2428 | GAGTAGATGAATGAATTAATGAATA |
| 1165 | TTATTCATTAATTCATTCATCTACT | 2429 | AGTAGATGAATGAATTAATGAATAA |
| 1166 | TTTATTCATTAATTCATTCATCTAC | 2430 | GTAGATGAATGAATTAATGAATAAA |
| 1167 | GTTTATTCATTAATTCATTCATCTA | 2431 | TAGATGAATGAATTAATGAATAAAC |
| 1168 | TGTTTATTCATTAATTCATTCATCT | 2432 | AGATGAATGAATTAATGAATAAACA |
| 1169 | CTGTTTATTCATTAATTCATTCATC | 2433 | GATGAATGAATTAATGAATAAACAG |
| 1170 | CCTGTTTATTCATTAATTCATTCAT | 2434 | ATGAATGAATTAATGAATAAACAGG |
| 1171 | GCCTGTTTATTCATTAATTCATTCA | 2435 | TGAATGAATTAATGAATAAACAGGC |
| 1172 | TGCCTGTTTATTCATTAATTCATTC | 2436 | GAATGAATTAATGAATAAACAGGCA |
| 1173 | CTGCCTGTTTATTCATTAATTCATT | 2437 | AATGAATTAATGAATAAACAGGCAG |
| 1174 | TCTGCCTGTTTATTCATTAATTCAT | 2438 | ATGAATTAATGAATAAACAGGCAGA |
| 1175 | ATCTGCCTGTTTATTCATTAATTCA | 2439 | TGAATTAATGAATAAACAGGCAGAT |
| 1176 | CATCTGCCTGTTTATTCATTAATTC | 2440 | GAATTAATGAATAAACAGGCAGATG |
| 1177 | CCATCTGCCTGTTTATTCATTAATT | 2441 | AATTAATGAATAAACAGGCAGATGG |
| 1178 | TCCATCTGCCTGTTTATTCATTAAT | 2442 | ATTAATGAATAAACAGGCAGATGGA |
| 1179 | ATCCATCTGCCTGTTTATTCATTAA | 2443 | TTAATGAATAAACAGGCAGATGGAT |
| 1180 | CATCCATCTGCCTGTTTATTCATTA | 2444 | TAATGAATAAACAGGCAGATGGATG |
| 1181 | TCATCCATCTGCCTGTTTATTCATT | 2445 | AATGAATAAACAGGCAGATGGATGA |
| 1182 | ATCATCCATCTGCCTGTTTATTCAT | 2446 | ATGAATAAACAGGCAGATGGATGAT |
| 1183 | CATCATCCATCTGCCTGTTTATTCA | 2447 | TGAATAAACAGGCAGATGGATGATG |
| 1184 | ACATCATCCATCTGCCTGTTTATTC | 2448 | GAATAAACAGGCAGATGGATGATGT |
| 1185 | TACATCATCCATCTGCCTGTTTATT | 2449 | AATAAACAGGCAGATGGATGATGTA |
| 1186 | TTACATCATCCATCTGCCTGTTTAT | 2450 | ATAAACAGGCAGATGGATGATGTAA |
| 1187 | CTTACATCATCCATCTGCCTGTTTA | 2451 | TAAACAGGCAGATGGATGATGTAAG |
| 1188 | GCTTACATCATCCATCTGCCTGTTT | 2452 | AAACAGGCAGATGGATGATGTAAGC |
| 1189 | AGCTTACATCATCCATCTGCCTGTT | 2453 | AACAGGCAGATGGATGATGTAAGCT |
| 1190 | CAGCTTACATCATCCATCTGCCTGT | 2454 | ACAGGCAGATGGATGATGTAAGCTG |
| 1191 | GCAGCTTACATCATCCATCTGCCTG | 2455 | CAGGCAGATGGATGATGTAAGCTGC |
| 1192 | GGCAGCTTACATCATCCATCTGCCT | 2456 | AGGCAGATGGATGATGTAAGCTGCC |
| 1193 | GGGCAGCTTACATCATCCATCTGCC | 2457 | GGCAGATGGATGATGTAAGCTGCCC |
| 1194 | GGGGCAGCTTACATCATCCATCTGC | 2458 | GCAGATGGATGATGTAAGCTGCCCC |
| 1195 | TGGGGCAGCTTACATCATCCATCTG | 2459 | CAGATGGATGATGTAAGCTGCCCCA |
| 1196 | CTGGGGCAGCTTACATCATCCATCT | 2460 | AGATGGATGATGTAAGCTGCCCCAG |
| 1197 | TCTGGGGCAGCTTACATCATCCATC | 2461 | GATGGATGATGTAAGCTGCCCCAGA |
| 1198 | GTCTGGGGCAGCTTACATCATCCAT | 2462 | ATGGATGATGTAAGCTGCCCCAGAC |
| 1199 | GGTCTGGGGCAGCTTACATCATCCA | 2463 | TGGATGATGTAAGCTGCCCCAGACC |
| 1200 | GGGTCTGGGGCAGCTTACATCATCC | 2464 | GGATGATGTAAGCTGCCCCAGACCC |
| 1201 | AGGGTCTGGGGCAGCTTACATCATC | 2465 | GATGATGTAAGCTGCCCCAGACCCT |
| 1202 | CAGGGTCTGGGGCAGCTTACATCAT | 2466 | ATGATGTAAGCTGCCCCAGACCCTG |
| 1203 | CCAGGGTCTGGGGCAGCTTACATCA | 2467 | TGATGTAAGCTGCCCCAGACCCTGG |
| 1204 | CCCAGGGTCTGGGGCAGCTTACATC | 2468 | GATGTAAGCTGCCCCAGACCCTGGG |
| 1205 | TCCCAGGGTCTGGGGCAGCTTACAT | 2469 | ATGTAAGCTGCCCCAGACCCTGGGA |
| 1206 | GTCCCAGGGTCTGGGGCAGCTTACA | 2470 | TGTAAGCTGCCCCAGACCCTGGGAC |
| 1207 | GGTCCCAGGGTCTGGGGCAGCTTAC | 2471 | GTAAGCTGCCCCAGACCCTGGGACC |
| 1208 | AGGTCCCAGGGTCTGGGGCAGCTTA | 2472 | TAAGCTGCCCCAGACCCTGGGACCT |
| 1209 | GAGGTCCCAGGGTCTGGGGCAGCTT | 2473 | AAGCTGCCCCAGACCCTGGGACCTC |
| 1210 | AGAGGTCCCAGGGTCTGGGGCAGCT | 2474 | AGCTGCCCCAGACCCTGGGACCTCT |
| 1211 | CAGAGGTCCCAGGGTCTGGGGCAGC | 2475 | GCTGCCCCAGACCCTGGGACCTCTG |
| 1212 | TCAGAGGTCCCAGGGTCTGGGGCAG | 2476 | CTGCCCCAGACCCTGGGACCTCTGA |
| 1213 | GTCAGAGGTCCCAGGGTCTGGGGCA | 2477 | TGCCCCAGACCCTGGGACCTCTGAC |
| 1214 | GGTCAGAGGTCCCAGGGTCTGGGGC | 2478 | GCCCCAGACCCTGGGACCTCTGACC |
| 1215 | GGGTCAGAGGTCCCAGGGTCTGGGG | 2479 | CCCCAGACCCTGGGACCTCTGACCC |
| 1216 | GGGGTCAGAGGTCCCAGGGTCTGGG | 2480 | CCCAGACCCTGGGACCTCTGACCCC |
| 1217 | GGGGGTCAGAGGTCCCAGGGTCTGG | 2481 | CCAGACCCTGGGACCTCTGACCCCC |
| 1218 | CGGGGGTCAGAGGTCCCAGGGTCTG | 2482 | CAGACCCTGGGACCTCTGACCCCCG |
| 1219 | CCGGGGGTCAGAGGTCCCAGGGTCT | 2483 | AGACCCTGGGACCTCTGACCCCCGG |
| 1220 | GCCGGGGGTCAGAGGTCCCAGGGTC | 2484 | GACCCTGGGACCTCTGACCCCCGGC |
| 1221 | CGCCGGGGGTCAGAGGTCCCAGGGT | 2485 | ACCCTGGGACCTCTGACCCCCGGCG |
| 1222 | TCGCCGGGGGTCAGAGGTCCCAGGG | 2486 | CCCTGGGACCTCTGACCCCCGGCGA |
| 1223 | GTCGCCGGGGGTCAGAGGTCCCAGG | 2487 | CCTGGGACCTCTGACCCCCGGCGAC |
| 1224 | GGTCGCCGGGGGTCAGAGGTCCCAG | 2488 | CTGGGACCTCTGACCCCCGGCGACC |
| 1225 | GGGTCGCCGGGGGTCAGAGGTCCCA | 2489 | TGGGACCTCTGACCCCCGGCGACCC |
| 1226 | GGGGTCGCCGGGGGTCAGAGGTCCC | 2490 | GGGACCTCTGACCCCCGGCGACCCC |
| 1227 | AGGGGTCGCCGGGGGTCAGAGGTCC | 2491 | GGACCTCTGACCCCCGGCGACCCCT |
| 1228 | AAGGGGTCGCCGGGGGTCAGAGGTC | 2492 | GACCTCTGACCCCCGGCGACCCCTT |
| 1229 | CAAGGGGTCGCCGGGGGTCAGAGGT | 2493 | ACCTCTGACCCCCGGCGACCCCTTG |
| 1230 | GCAAGGGGTCGCCGGGGGTCAGAGG | 2494 | CCTCTGACCCCCGGCGACCCCTTGC |
| 1231 | TGCAAGGGGTCGCCGGGGGTCAGAG | 2495 | CTCTGACCCCCGGCGACCCCTTGCA |
| 1232 | GTGCAAGGGGTCGCCGGGGGTCAGA | 2496 | TCTGACCCCCGGCGACCCCTTGCAC |
| 1233 | AGTGCAAGGGGTCGCCGGGGGTCAG | 2497 | CTGACCCCCGGCGACCCCTTGCACT |
| 1234 | GAGTGCAAGGGGTCGCCGGGGGTCA | 2498 | TGACCCCCGGCGACCCCTTGCACTC |
| 1235 | AGAGTGCAAGGGGTCGCCGGGGGTC | 2499 | GACCCCCGGCGACCCCTTGCACTCT |
| 1236 | GAGAGTGCAAGGGGTCGCCGGGGGT | 2500 | ACCCCCGGCGACCCCTTGCACTCTC |
| 1237 | GGAGAGTGCAAGGGGTCGCCGGGGG | 2501 | CCCCCGGCGACCCCTTGCACTCTCC |
| 1238 | TGGAGAGTGCAAGGGGTCGCCGGGG | 2502 | CCCCGGCGACCCCTTGCACTCTCCA |
| 1239 | ATGGAGAGTGCAAGGGGTCGCCGGG | 2503 | CCCGGCGACCCCTTGCACTCTCCAT |
| 1240 | CATGGAGAGTGCAAGGGGTCGCCGG | 2504 | CCGGCGACCCCTTGCACTCTCCATG |
| 1241 | TCATGGAGAGTGCAAGGGGTCGCCG | 2505 | CGGCGACCCCTTGCACTCTCCATGA |
| 1242 | GTCATGGAGAGTGCAAGGGGTCGCC | 2506 | GGCGACCCCTTGCACTCTCCATGAC |
| 1243 | TGTCATGGAGAGTGCAAGGGGTCGC | 2507 | GCGACCCCTTGCACTCTCCATGACA |
| 1244 | GTGTCATGGAGAGTGCAAGGGGTCG | 2508 | CGACCCCTTGCACTCTCCATGACAC |
| 1245 | AGTGTCATGGAGAGTGCAAGGGGTC | 2509 | GACCCCTTGCACTCTCCATGACACT |
| 1246 | AAGTGTCATGGAGAGTGCAAGGGGT | 2510 | ACCCCTTGCACTCTCCATGACACTT |
| 1247 | AAAGTGTCATGGAGAGTGCAAGGGG | 2511 | CCCCTTGCACTCTCCATGACACTTT |
| 1248 | GAAAGTGTCATGGAGAGTGCAAGGG | 2512 | CCCTTGCACTCTCCATGACACTTTC |
| 1249 | AGAAAGTGTCATGGAGAGTGCAAGG | 2513 | CCTTGCACTCTCCATGACACTTTCT |
| 1250 | GAGAAAGTGTCATGGAGAGTGCAAG | 2514 | CTTGCACTCTCCATGACACTTTCTC |
| 1251 | AGAGAAAGTGTCATGGAGAGTGCAA | 2515 | TTGCACTCTCCATGACACTTTCTCT |
| 1252 | GAGAGAAAGTGTCATGGAGAGTGCA | 2516 | TGCACTCTCCATGACACTTTCTCTC |
| 1253 | GGAGAGAAAGTGTCATGGAGAGTGC | 2517 | GCACTCTCCATGACACTTTCTCTCC |
| 1254 | GGGAGAGAAAGTGTCATGGAGAGTG | 2518 | CACTCTCCATGACACTTTCTCTCCC |
| 1255 | TGGGAGAGAAAGTGTCATGGAGAGT | 2519 | ACTCTCCATGACACTTTCTCTCCCA |
| 1256 | ATGGGAGAGAAAGTGTCATGGAGAG | 2520 | CTCTCCATGACACTTTCTCTCCCAT |
| 1257 | CATGGGAGAGAAAGTGTCATGGAGA | 2521 | TCTCCATGACACTTTCTCTCCCATG |
| 1258 | CCATGGGAGAGAAAGTGTCATGGAG | 2522 | CTCCATGACACTTTCTCTCCCATGG |
| 1259 | ACCATGGGAGAGAAAGTGTCATGGA | 2523 | TCCATGACACTTTCTCTCCCATGGT |
| 1260 | CACCATGGGAGAGAAAGTGTCATGG | 2524 | CCATGACACTTTCTCTCCCATGGTG |
| 1261 | CCACCATGGGAGAGAAAGTGTCATG | 2525 | CATGACACTTTCTCTCCCATGGTGG |
| 1262 | GCCACCATGGGAGAGAAAGTGTCAT | 2526 | ATGACACTTTCTCTCCCATGGTGGC |
| 1263 | TGCCACCATGGGAGAGAAAGTGTCA | 2527 | TGACACTTTCTCTCCCATGGTGGCA |
| 1264 | CTGCCACCATGGGAGAGAAAGTGTC | 2528 | GACACTTTCTCTCCCATGGTGGCAG |

*At least one (i.e., one or more) nucleoside linkage of the oligonucleotide sequence is independently selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

### UNC13A Transcript

In various embodiments, an UNC13A mRNA transcript comprises the sequence
provided as SEQ ID NO: 5057.
GCCCCCGGTGCTGAACCAAGATGGCCGGTGGCGGCCGGGCCCCGGCGTGAGCCAAGCGCGGGCTGCAGCC
GGGAGATGCCCCAGCCCAGCGGCCGCTGAGCCCGACCCGACAGAGCCGGCCCGGCCGCCTCCGGCCCACC
TGCGAGCTCGGAGACATGTCTCTGCTTTGCGTTGGAGTCAAAAAAGCCAAGTTTGATGGTGCCCAAGAGA
AATTCAACACGTACGTGACCCTGAAAGTGCAGAATGTCAAGAGCACGACCATCGCGGTGCGGGGCAGCCA
GCCCAGCTGGGAGCAGGATTTCATGTTCGAGATTAACCGTCTGGATTTGGGACTGACGGTGGAGGTGTGG
AATAAGGGTCTCATCTGGGACACAATGGTGGGCACTGTGTGGATCCCACTGAGGACCATCCGCCAGTCCA
ATGAGGAGGGCCCTGGAGAGTGGCTGACGCTGGACTCCCAGGTCATCATGGCAGACAGTGAGATCTGTGG
CACCAAGGACCCCACCTTCCACCGCATCCTCCTGGACACGCGCTTTGAGCTACCCTTAGACATTCCTGAA
GAGGAGGCTCGCTACTGGGCCAAGAAGCTGGAGCAGCTCAATGCTATGCGGGACCAGGATGAATATTCGT
TCCAAGATGAGCAAGACAAGCCTCTGCCTGTCCCCAGCAACCAGTGCTGCAACTGGAATTATTTTGGCTG
GGGTGAGCAGCACAACGATGACCCCGACAGTGCAGTGGATGATCGTGACAGTGACTACCGCAGTGAAACG
AGCAACAGCATCCCGCCGCCCTATTATACTACGTCACAACCCAACGCCTCAGTCCACCAATATTCTGTTC
GCCCACCACCCCTGGGCTCCCGGGAGTCCTACAGTGACTCCATGCACAGTTACGAGGAGTTCTCTGAGCC
ACAAGCCCTCAGCCCCACGGGTAGCAGCCGCTATGCCTCTTCCGGGGAGCTGAGCCAGGGAAGCTCTCAG
CTGAGCGAGGACTTCGACCCTGACGAGCACAGCCTGCAGGGCTCCGACATGGAGGATGAGCGGGACCGGG
ACTCCTACCACTCCTGCCACAGCTCGGTCAGCTACCACAAAGACTCGCCTCGCTGGGACCAGGATGAGGA
AGAGCTGGAGGAGGACCTGGAGGACTTCCTGGAGGAGGAGGAGCTGCCTGAAGATGAGGAGGAGCTGGAG
GAGGAGGAGGAGGAGGTGCCTGACGATTTGGGCAGCTATGCCCAGCGTGAAGACGTAGCTGTGGCTGAGC
CCAAAGACTTCAAACGCATCAGCCTCCCGCCAGCTGCCCCAGGGAAGGAGGACAAGGCCCCAGTGGCACC
CACCGAGGCCCCCGACATGGCCAAGGTGGCCCCCAAGCCAGCCACGCCCGACAAGGTGCCTGCAGCTGAG
CAGATCCCTGAGGCTGAGCCACCCAAGGACGAGGAGAGTTTCAGGCCGAGAGAGGATGAGGAAGGCCAGG
AGGGGCAGGACTCCATGTCCAGGGCCAAGGCCAACTGGCTGCGTGCCTTCAACAAGGTGCGGATGCAGCT
GCAGGAGGCCCGGGGAGAAGGAGAGATGTCTAAATCCCTATGGTTCAAAGGCGGCCCAGGGGGCGGTCTC
ATCATCATCGACAGCATGCCAGACATCCGCAAGAGGAAACCTATCCCACTCGTGAGCGACTTGGCCATGT
CCCTGGTCCAGTCCAGGAAAGCGGGCATCACCTCGGCCTTGGCCTCCAGCACGTTGAACAACGAGGAGCT
GAAAAACCACGTTTACAAGAAGACCCTGCAAGCCTTAATCTACCCCATCTCGTGCACGACGCCACACAAC
TTCGAAGTGTGGACGGCCACCACGCCCACCTACTGCTACGAGTGCGAGGGGCTGCTGTGGGGCATCGCGA
GGCAGGGCATGCGCTGCACCGAGTGCGGTGTCAAGTGCCACGAGAAGTGCCAGGACCTGCTCAACGCCGA
CTGCCTGCAGCGGGCTGCGGAGAAGAGCTCCAAGCACGGGGCGGAGGACCGGACACAGAACATCATCATG
GTGCTCAAGGACCGCATGAAGATCCGGGAGCGCAACAAGCCCGAGATCTTCGAGCTCATCCAGGAGATCT
TCGCGGTGACCAAGACGGCGCACACGCAGCAGATGAAGGCGGTCAAGCAGAGCGTGCTGGACGGCACGTC
CAAGTGGTCCGCCAAGATCAGCATCACCGTGGTCTGCGCCCAGGGCTTGCAGGCAAAGGACAAGACAGGA
TCCAGTGACCCCTATGTCACCGTCCAGGTCGGGAAGACCAAGAAACGGACAAAAACCATCTATGGGAACC
TCAACCCGGTGTGGGAGGAGAATTTCCACTTTGAATGTCACAATTCCTCCGACCGCATCAAGGTGCGCGT
CTGGGACGAGGATGACGACATCAAATCCCGCGTGAAACAGAGGTTCAAGAGGGAATCTGACGATTTCCTG
GGGCAGACGATCATTGAGGTGCGGACGCTCAGCGGCGAGATGGACGTGTGGTACAACCTGGACAAGCGAA
CTGACAAATCTGCCGTGTCGGGTGCCATCCGGCTCCACATCAGTGTGGAGATCAAAGGCGAGGAGAAGGT
GGCCCCGTACCATGTCCAGTACACCTGTCTGCATGAGAACCTGTTCCACTTCGTGACCGACGTGCAGAAC
AATGGGGTCGTGAAGATCCCAGATGCCAAGGGTGACGATGCCTGGAAGGTTTACTACGATGAGACAGCCC
AGGAGATTGTGGACGAGTTTGCCATGCGCTACGGCGTCGAGTCCATCTACCAAGCCATGACCCACTTTGC
CTGCCTCTCCTCCAAGTATATGTGCCCAGGGGTGCCTGCCGTCATGAGCACCCTGCTCGCCAACATCAAT
GCCTACTACGCACACACCACCGCCTCCACCAACGTGTCTGCCTCCGACCGCTTCGCCGCCTCCAACTTTG
GGAAAGAGCGCTTCGTGAAACTCCTGGACCAGCTGCATAACTCCCTGCGGATTGACCTCTCCATGTACCG
GAATAACTTCCCAGCCAGCAGCCCGGAGAGACTCCAGGACCTCAAATCCACTGTGGACCTTCTCACCAGC
ATCACCTTCTTTCGGATGAAGGTACAAGAACTCCAGAGCCCGCCCCGAGCCAGCCAGGTGGTAAAGGACT
GTGTGAAAGCCTGCCTTAATTCTACCTACGAGTACATCTTCAATAACTGCCATGAACTGTACAGCCGGGA
GTACCAGACAGACCCGGCCAAGAAGGGGGAAGTTCTCCCAGAGGAACAGGGGCCCAGCATCAAGAACCTC
GACTTCTGGTCCAAGCTGATTACCCTCATAGTGTCCATCATTGAGGAAGACAAGAATTCCTACACTCCCT
GCCTCAACCAGTTTCCCCAGGAGCTGAATGTGGGTAAAATCAGCGCTGAAGTGATGTGGAATCTGTTTGC
CCAAGACATGAAGTACGCCATGGAGGAGCACGACAAGCATCGTCTATGCAAGAGTGCCGACTACATGAAC
CTCCACTTCAAGGTGAAATGGCTCTACAATGAGTATGTGACGGAACTTCCCGCCTTCAAGGACCGCGTGC
CTGAGTACCCTGCATGGTTTGAACCCTTCGTCATCCAGTGGCTGGATGAGAATGAGGAGGTGTCCCGGGA
TTTCCTGCACGGTGCCCTGGAGCGAGACAAGAAGGATGGGTTCCAGCAGACCTCAGAGCATGCCCTATTC
TCCTGCTCCGTGGTGGATGTTTTCTCCCAACTCAACCAGAGCTTTGAAATCATCAAGAAACTCGAGTGTC
CCGACCCTCAGATCGTGGGGCACTACATGAGGCGCTTTGCCAAGACCATCAGTAATGTGCTCCTCCAGTA
TGCAGACATCATCTCCAAGGACTTTGCCTCCTACTGCTCCAAGGAGAAGGAGAAAGTGCCCTGCATTCTC
ATGAATAACACTCAACAGCTACGAGTTCAGCTGGAGAAGATGTTCGAAGCCATGGGAGGAAAGGAGCTGG
ATGCTGAAGCCAGTGACATCCTGAAGGAGCTTCAGGTGAAACTCAATAACGTCTTGGATGAGCTCAGCCG
GGTGTTTGCTACCAGCTTCCAGCCGCACATTGAAGAGTGTGTCAAACAGATGGGTGACATCCTTAGCCAG
GTTAAGGGCACAGGCAATGTGCCAGCCAGTGCCTGCAGCAGCGTGGCCCAGGACGCGGACAATGTGTTGC
AGCCCATCATGGACCTGCTGGACAGCAACCTGACCCTCTTTGCCAAAATCTGTGAGAAGACTGTGCTGAA
GCGAGTGCTGAAGGAGCTGTGGAAGCTGGTTATGAACACCATGGAGAAAACCATCGTCCTGCCGCCCCTC
ACTGACCAGACGATGATCGGGAACCTCTTGAGAAAACATGGCAAGGGATTAGAAAAGGGCAGGGTGAAAT
TGCCAAGCCACTCAGACGGAACCCAGATGATCTTCAATGCAGCCAAGGAGCTGGGTCAGCTGTCCAAACT
CAAGGATCACATGGTACGAGAAGAAGCCAAGAGCTTGACCCCAAAGCAGTGCGCGGTTGTTGAGTTGGCC
CTGGACACCATCAAGCAATATTTCCACGCGGGTGGCGTGGGCCTCAAGAAGACCTTCCTGGAGAAGAGCC
CGGACCTGCAATCCTTGCGCTATGCCCTGTCGCTCTACACGCAGGCCACCGACCTGCTAATCAAGACCTT
TGTACAGACGCAATCGGCCCAGGGCTTGGGTGTAGAAGACCCTGTGGGTGAAGTCTCTGTCCATGTTGAG
CTGTTCACTCATCCAGGAACTGGGGAACACAAGGTCACAGTGAAAGTGGTGGCTGCCAATGACCTCAAGT
GGCAGACTTCTGGCATCTTCCGGCCGTTCATCGAGGTCAACATCATTGGGCCCCAGCTCAGCGACAAGAA
ACGCAAGTTTGCGACCAAATCCAAGAACAATAGCTGGGCTCCCAAGTACAATGAGAGCTTCCAGTTCACG
CTGAGCGCCGACGCGGGTCCCGAGTGCTATGAGCTGCAGGTGTGCGTCAAGGACTACTGCTTCGCGCGCG
AGGACCGCACGGTGGGGCTGGCCGTGCTGCAGCTGCGTGAGCTGGCCCAGCGCGGGAGCGCCGCCTGCTG
GCTGCCGCTCGGCCGCCGCATCCACATGGACGACACGGGCCTCACGGTGCTGCGAATCCTCTCGCAGCGC
AGCAACGACGAGGTGGCCAAGGAGTTCGTGAAGCTCAAGTCGGACACGCGCTCCGCCGAGGAGGGCGGTG
CCGCGCCTGCGCCTTAGCGCGGGCGGTCGGCCGAGCGGCACTGCGCCTGCGCGGAGGGCGCTGGGCGGGG
AGGGACGGGGCTTGCGCCTTGGTGGGACCTCCCCAGGGGCGGGGCTCGGGGGGCTCCACGCCAAGGGTGG
GCTGCGCCTACGCCCTTGACTCAGCTTTCCCTTTTGGGGAATTAGGAATGGAGGATGCCCCGCCCTCTCG
GGAGGCCACGCCCAAGGGCGCGACGAAGGAAGGAGCCACATCCCCAACTTGAGGCCACGCCCCCAGCACC
TAGGGGGCATTTTGAGCTGGGATGGGGGAAACCTCGTCCCTATGGAGGAGGCCACATCCCGGGGCTCTGG
TACCGGGAGGCACCACCTCATGTCCCCTGGAAAAGCCATAAGATGGGACCCAGACCCCTGGGACCCCAGA
CCAATTGCCAAGTATGGAAATCTCAGCTCCCTCGAGGGGGGGCCCTGGGCAAGGGGTAGGGCTCTCTGGA
GCGCCCCTCTAGGTGGCCTGGGGACTGGAGGGACCAGGATGCTGGTTGGAGGGCCCCGGAATACCGGAGT
CCCTTTAGATATTTGTGCAAAAAATAAATGGGGGGAGGGGGGAGGATGGGATTTCAAAAGCACATGCGCC
CTTGGGCGCCCAAACCCTGGGGGCCGAGGGGACGGCTCTGGTTCCCCACGCTGCCCCTACTTCCCTTTGG
GAGTTTGCCTCTCCCTCTCCCCCAACAAACCCAGTCCTCATATCATAGAGTTCAACACACCCATTTGACA
GATGGCAAAACTGAGGCTTAAAGAGCTGCTTGAGACTTGGCCAAGGTTCCAGGTGCCATACCCTCTGTGC
CCCTCCCTTAGGCCTGTGTGCCCCATGGAAGGGTGGGCTGAGATCGGGATGACCTGACACAGCTCCCTAT
TGCTGCTAATTCCCCCTCGGCCTCCTCCAAGGGGTGGGAATTCCAGGCCAAGACCCCTACTTCGCCTTTC
CTTCTCCGGCTGCCAAGCAGGACCTTTGCCCTCAGCCCTTTCTCCTGGGATCTCCATGGGGGATGCCATG
AGGGCCTCCCACCACAAAAGAGAATTTGGGATCCCCTGGTCCCAGGTTTCTCCATCCCTTCTTCCTTTTC
CAGAATTTTCCAAATAGGAAAGAACAGAAGGAGACCAGAAACTCTAGGGGGGAGAAAGAGAATGAGAGAA
AGAGAATGAGAGAGAGAGAAACACAAACACAGTGACACAGTGAGAGCTTAGTCTCCAAGAGCCTATTCAT
TGATTCAAACACCCAAGCCACAGGATACCTCAGATGGCCCTCTTGCCAGCTGGAAGCTCTTTCTCCAATG
AGCAAAGTTACAGTGACCTGGCTGGAGTTACCTGGTGCACATAGGACCTTAGGGGAAAGTTCAGCGTGGA
CTACACTTGCTCTGGGATCTGCTTTTCCACATGTGTGTATGGCACGCCTTTTTCTGCTGGATTGGGAAGG
ACAAGATTTTGCTGTGCTAGGGAGAAATGAAAACGGGGTGAGCTGAGTAGCTGGGTTTCTGGAGGATAGA
ACATCAGATGGGGAGGCTTTCCGAGGTGAAGAATGAGAGGGAACCACTTACTAGAGAGAAAAGAGCTCCA
GGCCTGGGGAACAGCACGTGCGAAGGCCAGGAGAGAAGAACTGTTGAAACAACGAGAAGGGTGGCACGGC
TGGAGCTGAGCCAGCAAGGGGGATCGTGAGGAGCCTTGGGGTTGGGGAGATCTGCAGAAGCATCAGACCA
GGCAGGGCCTCGTACGCAGTCCTGAGGAGTTTTACTTTTATTCTAAGACAGTTGGGGAGCTCCAGGAGCT
GTTTTAAGTTGGGGAGAGACTGGATTCCAGCCTGCAAAAGCTGTTTTGTGAAGACTAAAACCAGTGAGGA
GAGGTGGAGGTGCTTTGGGGACACTGAAATGGATTCTTGGAAAGATTCTGAAGGCTGTGTTGAAAAGACA
CCTATAGCTGTGGGGACATGACTATAATCCCAGCATTTGGGGAGACCGAGGCTGGCAGATCACTTAAGGT
CAGGAGTTTGAGACCAGCCTGGCCAACATGGCGAAACCCCATCTCTGCTAAAAATACAAAAATTAGCTGG
GTGCAGTGGTGCATGCCTGTAGTCCCAGCTACTCAGGAGACTGAGGCGGGAGAATTGTTTGAACCCTGGA
GGCAGAGGTTGTAGTGAGTCGTGATCACACAACTGCACTCCAGCCTGGGCAACAGAACAATACTCCATTC
CCTCCCCTCTACCCCACCAAAAAAAAAAAAAAATCCTGCCCTTAGATGAGCTCTAGGGCTGCTGAGTACA
GTTGTCCCAGTTGCACAGTGCCCAAGGGTTTGGCATTGCTAAGAAGGCCACGTGCAAATCCTAGATATTG
AGTGTTGTATGTTTGTGACGTTGGTTTCCCGACATGTGAATGGCCCAAGTGTCTGGAAGAAGTGGCGCCA
CTTTCTAATTTGCTTGGAGATGTTGCATGTCCCTTAAATTCAGACAGGTGCAGGTAACTGGAGGTTCTGA
ACCAAAGGTTAAAATGCAAATTCTCATACAGGGTTGGGAAGTTGTAGCCAGGGATAAGCTTATGTGACTG
TTATATGGACTGAGGAGCAGATGTGAATTTCGAACCATGACATGGCTGAGGGTAGGGGTCGGGTGGATGG
ATGATTCAGGGTTGTAACCCATAGAGCCCAAAGGGGAAGTGATCTGTGACCTGGGGTGAGGGTGATCTGG
AAGATTTTTGGATGGCTGGAAAGAAATGGGGAAGTCGAGCTGCCTGAGAGAGCCAAGTTATTTCCCAAAA
GATTCCTTAGGAGTCTTTCTGTTCAAGACCTCCGTGTGTGTGTGTGTGTGTTTAGGGTTCCCCAGCAATG
GCCCAGGCATGTGAAGGAAACAAGCTTCTTCAGGGAATATTTGTTGAATGAGTTTTCCTGACTCCCAGGC
TAGAACTGTTTTTGCAATTTCCACCCTCTTTTCTTTCCCCCAGAGAACTCCTATTCGTCCTTCAAAACCC
ATCACGGAAACCCCTCTTGGAGAAAACCCTCCTTCCTTCCCCTCAGGACTTTCCCAGCCACCGTCTCTCC
TCCAGTCCAGCCTGATGCCATGGGACTGGGGGTTTCTCTGTCCAGCTCTGTTTCTCCCAGACTGGGGTCT
GAGGACTCTCAGGACCCCCAACTTTACCTAGCACAGGCTGGGCACAAGTGGGTGACAGGGAGTCTACGCC
TAGTGGAATTATGTATTGGGGCAGGGTCAGTGTGAGAATACACATCCGCATGCATGTCTGTCCATGTCTG
TCCGTACCAACCTTCCCCTTCCACACGGACCTGGGCACATAGGAGGTGTCTGAGCCTGACACATGGGACA
GAGAGTGGACATGGCTGAGACACGGACAGAGAAAAGACAAGGAGTCCAGGGGGCTGAAAGCCTTTTGAAA
TCAGGAAGTTCCTGTATTGGCAGAACAAAGCCCAGAGAGGAGCAGGGCTTTCCTCAACGCCACCCAGCAA
GTGGACACAGAGCCCGGCCTTGGATGACACCTCCAGGGTTCTGAACCCTGGACCTCGCTTTATGCAAGGA
GCTGGCCCCACATTTCCATGAATCGGGGAAACAGCACAAGAAGGTTGGCCTGTGGCAGGGCAAGGGTTAA
AGGGGTGACATTGAGGGATGCCTCAGAGTCAAAGTCCCCTGACCAAGAGGAATAGAGTAGAAAACACAGA
GACAGAGGGTGAGATCACGCCCCGATGAGGACGGAGAGAGACAGAGATGGAGAGAGACATAGAGGTGGAA
ATATACAGAGAAAGATAAATGCAGAGACCAAGGCAGGGAGTGTCGGGGGAAGTAAAGAGGGTGTCCTGAA
GAAAGAAGGATCTGTTCACTCTTACCAGTCTGTCCTCGAATGATTTGCATAAAATGAGGAGGTGCCTGTC
CACACCCCCAATTCCTCTCTCAGGCCCCAGAGCCTGAGACCTCACCATGCCCCCATCAGAGATGCAAAAA
ACTAAACACCCAACTAGAAATCCTTGGGACCTCTCTCGGCTGGGATCTCAGAGCCTTTCTGTCCCCTACC
CCTACCCCATGTGCTGTCGATTTTGCAGATGGGGACAACCTGGGGCCTCCCGGAACTCTGCCACCCTGGG
GAAGTTGGGGGAGGGCCTTAGTCCCGGATCACAACCCCGTCTGCTCCCCAGAATCCTTTCCTAAGAATCG
TTGAGGACCAAAGTTGTCTTTGCTGACACGTGTTGCTTTTCTCTTTGCCTTTTATTGTTTCAGAGAAAAA
TCAAGTTGACTGTGTCAAGTAACACCCCACCCCTTACCCCCGTCCAGCCATAGTGGCTCTCTGGAGACAC
AGGTCACAGGCGGAGGGTCCCCTGATCATCCCCAACCACACAGCCAGGGGGACTTGACCCCTGTCCACCC
CTGTCTCGTGCTCCCTCAGACCCCCACAAACCGGCCAAGCAGTCCGGGGAGGCTTCCCCTCCACACAACT
CTTAGCATGTGATTGCAGATGTGAAATCAAAACGTTGTTTGTTTTTTGTTTTGTTTTGATTCTACCCCGT
CGGTCCAGTGTCTGCACAGACGCCTTCATTTCTCTGTAAATATGTGACTTGGAACAAATGTTTAACACAA
ACGAGAAGTGGTCATGAATGCATGGTGTTGAGATGTTTTGCACTATTCTGACTTTTTGGTCTCTGTAAAA
ATATTTTATTAACAGCAGACATTAAAAAAAGAAAAACCACACACA
(SEQ ID NO: 5057 (SOURCE NCBI Reference Sequence NM_001080421.3)).

In various embodiments, an UNC13A mRNA transcript comprises the sequence provided as SEQ ID NO: 5058.

GCCCCCGGTGCTGAACCAAGATGGCCGGTGGCGGCCGGGCCCCGGCGTGAGCCAAGCGCGGGCTGCAGCC
GGGAGATGCCCCAGCCCAGCGGCCGCTGAGCCCGACCCGACAGAGCCGGCCCGGCCGCCTCCGGCCCACC
TGCGAGCTCGGAGACATGTCTCTGCTTTGCGTTGGAGTCAAAAAAGCCAAGTTTGATGGTGCCCAAGAGA
AATTCAACACGTACGTGACCCTGAAAGTGCAGAATGTCAAGAGCACGACCATCGCGGTGCGGGGCAGCCA
GCCCAGCTGGGAGCAGGATTTCATGTTCGAGATTAACCGTCTGGATTTGGGACTGACGGTGGAGGTGTGG
AATAAGGGTCTCATCTGGGACACAATGGTGGGCACTGTGTGGATCCCACTGAGGACCATCCGCCAGTCCA
ATGAGGAGGGCCCTGGAGAGTGGCTGACGCTGGACTCCCAGGTCATCATGGCAGACAGTGAGATCTGTGG
CACCAAGGACCCCACCTTCCACCGCATCCTCCTGGACACGCGCTTTGAGCTACCCTTAGACATTCCTGAA
GAGGAGGCTCGCTACTGGGCCAAGAAGCTGGAGCAGCTCAATGCTATGCGGGACCAGGATGAATATTCGT
TCCAAGATGAGCAAGACAAGCCTCTGCCTGTCCCCAGCAACCAGTGCTGCAACTGGAATTATTTTGGCTG
GGGTGAGCAGCACAACGATGACCCCGACAGTGCAGTGGATGATCGTGACAGTGACTACCGCAGTGAAACG
AGCAACAGCATCCCGCCGCCCTATTATACTACGTCACAACCCAACGCCTCAGTCCACCAATATTCTGTTC
GCCCACCACCCCTGGGCTCCCGGGAGTCCTACAGTGACTCCATGCACAGTTACGAGGAGTTCTCTGAGCC
ACAAGCCCTCAGCCCCACGGGTAGCAGCCGCTATGCCTCTTCCGGGGAGCTGAGCCAGGGAAGCTCTCAG
CTGAGCGAGGACTTCGACCCTGACGAGCACAGCCTGCAGGGCTCCGACATGGAGGATGAGCGGGACCGGG
ACTCCTACCACTCCTGCCACAGCTCGGTCAGCTACCACAAAGACTCGCCTCGCTGGGACCAGGATGAGGA
AGAGCTGGAGGAGGACCTGGAGGACTTCCTGGAGGAGGAGGAGCTGCCTGAAGATGAGGAGGAGCTGGAG
GAGGAGGAGGAGGAGGTGCCTGACGATTTGGGCAGCTATGCCCAGCGTGAAGACGTAGCTGTGGCTGAGC
CCAAAGACTTCAAACGCATCAGCCTCCCGCCAGCTGCCCCAGGGAAGGAGGACAAGGCCCCAGTGGCACC
CACCGAGGCCCCCGACATGGCCAAGGTGGCCCCCAAGCCAGCCACGCCCGACAAGGTGCCTGCAGCTGAG
CAGATCCCTGAGGCTGAGCCACCCAAGGACGAGGAGAGTTTCAGGCCGAGAGAGGATGAGGAAGGCCAGG
AGGGGCAGGACTCCATGTCCAGGGCCAAGGCCAACTGGCTGCGTGCCTTCAACAAGGTGCGGATGCAGCT
GCAGGAGGCCCGGGGAGAAGGAGAGATGTCTAAATCCCTATGGTTCAAAGGCGGCCCAGGGGGCGGTCTC
ATCATCATCGACAGCATGCCAGACATCCGCAAGAGGAAACCTATCCCACTCGTGAGCGACTTGTCCCTGG
TCCAGTCCAGGAAAGCGGGCATCACCTCGGCCTTGGCCTCCAGCACGTTGAACAACGAGGAGCTGAAAAA
CCACGTTTACAAGAAGACCCTGCAAGCCTTAATCTACCCCATCTCGTGCACGACGCCACACAACTTCGAA
GTGTGGACGGCCACCACGCCCACCTACTGCTACGAGTGCGAGGGGCTGCTGTGGGGCATCGCGAGGCAGG
GCATGCGCTGCACCGAGTGCGGTGTCAAGTGCCACGAGAAGTGCCAGGACCTGCTCAACGCCGACTGCCT
GCAGCGGGCTGCGGAGAAGAGCTCCAAGCACGGGGCGGAGGACCGGACACAGAACATCATCATGGTGCTC
AAGGACCGCATGAAGATCCGGGAGCGCAACAAGCCCGAGATCTTCGAGCTCATCCAGGAGATCTTCGCGG
TGACCAAGACGGCGCACACGCAGCAGATGAAGGCGGTCAAGCAGAGCGTGCTGGACGGCACGTCCAAGTG
GTCCGCCAAGATCAGCATCACCGTGGTCTGCGCCCAGGGCTTGCAGGCAAAGGACAAGACAGGATCCAGT
GACCCCTATGTCACCGTCCAGGTCGGGAAGACCAAGAAACGGACAAAAACCATCTATGGGAACCTCAACC
CGGTGTGGGAGGAGAATTTCCACTTTGAATGTCACAATTCCTCCGACCGCATCAAGGTGCGCGTCTGGGA
CGAGGATGACGACATCAAATCCCGCGTGAAACAGAGGTTCAAGAGGGAATCTGACGATTTCCTGGGGCAG
ACGATCATTGAGGTGCGGACGCTCAGCGGCGAGATGGACGTGTGGTACAACCTGGACAAGCGAACTGACA
AATCTGCCGTGTCGGGTGCCATCCGGCTCCACATCAGTGTGGAGATCAAAGGCGAGGAGAAGGTGGCCCC
GTACCATGTCCAGTACACCTGTCTGCATGAGAACCTGTTCCACTTCGTGACCGACGTGCAGAACAATGGG
GTCGTGAAGATCCCAGATGCCAAGGGTGACGATGCCTGGAAGGTTTACTACGATGAGACAGCCCAGGAGA
TTGTGGACGAGTTTGCCATGCGCTACGGCGTCGAGTCCATCTACCAAGCCATGACCCACTTTGCCTGCCT
CTCCTCCAAGTATATGTGCCCAGGGGTGCCTGCCGTCATGAGCACCCTGCTCGCCAACATCAATGCCTAC
TACGCACACACCACCGCCTCCACCAACGTGTCTGCCTCCGACCGCTTCGCCGCCTCCAACTTTGGGAAAG
AGCGCTTCGTGAAACTCCTGGACCAGCTGCATAACTCCCTGCGGATTGACCTCTCCATGTACCGGAATAA
CTTCCCAGCCAGCAGCCCGGAGAGACTCCAGGACCTCAAATCCACTGTGGACCTTCTCACCAGCATCACC
TTCTTTCGGATGAAGGTACAAGAACTCCAGAGCCCGCCCCGAGCCAGCCAGGTGGTAAAGGACTGTGTGA
AAGCCTGCCTTAATTCTACCTACGAGTACATCTTCAATAACTGCCATGAACTGTACAGCCGGGAGTACCA
GACAGACCCGGCCAAGAAGGGGGAAGTTCTCCCAGAGGAACAGGGGCCCAGCATCAAGAACCTCGACTTC
TGGTCCAAGCTGATTACCCTCATAGTGTCCATCATTGAGGAAGACAAGAATTCCTACACTCCCTGCCTCA
ACCAGTTTCCCCAGGAGCTGAATGTGGGTAAAATCAGCGCTGAAGTGATGTGGAATCTGTTTGCCCAAGA
CATGAAGTACGCCATGGAGGAGCACGACAAGCATCGTCTATGCAAGAGTGCCGACTACATGAACCTCCAC
TTCAAGGTGAAATGGCTCTACAATGAGTATGTGACGGAACTTCCCGCCTTCAAGGACCGCGTGCCTGAGT
ACCCTGCATGGTTTGAACCCTTCGTCATCCAGTGGCTGGATGAGAATGAGGAGGTGTCCCGGGATTTCCT
GCACGGTGCCCTGGAGCGAGACAAGAAGGATGGGTTCCAGCAGACCTCAGAGCATGCCCTATTCTCCTGC
TCCGTGGTGGATGTTTTCTCCCAACTCAACCAGAGCTTTGAAATCATCAAGAAACTCGAGTGTCCCGACC
CTCAGATCGTGGGGCACTACATGAGGCGCTTTGCCAAGACCATCAGTAATGTGCTCCTCCAGTATGCAGA
CATCATCTCCAAGGACTTTGCCTCCTACTGCTCCAAGGAGAAGGAGAAAGTGCCCTGCATTCTCATGAAT
AACACTCAACAGCTACGAGTTCAGCTGGAGAAGATGTTCGAAGCCATGGGAGGAAAGGAGCTGGATGCTG
AAGCCAGTGACATCCTGAAGGAGCTTCAGGTGAAACTCAATAACGTCTTGGATGAGCTCAGCCGGGTGTT
TGCTACCAGCTTCCAGCCGCACATTGAAGAGTGTGTCAAACAGATGGGTGACATCCTTAGCCAGGTTAAG
GGCACAGGCAATGTGCCAGCCAGTGCCTGCAGCAGCGTGGCCCAGGACGCGGACAATGTGTTGCAGCCCA
TCATGGACCTGCTGGACAGCAACCTGACCCTCTTTGCCAAAATCTGTGAGAAGACTGTGCTGAAGCGAGT
GCTGAAGGAGCTGTGGAAGCTGGTTATGAACACCATGGAGAAAACCATCGTCCTGCCGCCCCTCACTGAC
CAGACGGGGAACCTCTTGAGAAAACATGGCAAGGGATTAGAAAAGGGCAGGGTGAAATTGCCAAGCCACT
CAGACGGAACCCAGATGATCTTCAATGCAGCCAAGGAGCTGGGTCAGCTGTCCAAACTCAAGGATCACAT
GGTACGAGAAGAAGCCAAGAGCTTGACCCCAAAGCAGTGCGCGGTTGTTGAGTTGGCCCTGGACACCATC
AAGCAATATTTCCACGCGGGTGGCGTGGGCCTCAAGAAGACCTTCCTGGAGAAGAGCCCGGACCTGCAAT
CCTTGCGCTATGCCCTGTCGCTCTACACGCAGGCCACCGACCTGCTAATCAAGACCTTTGTACAGACGCA
ATCGGCCCAGGGCTTGGGTGTAGAAGACCCTGTGGGTGAAGTCTCTGTCCATGTTGAGCTGTTCACTCAT
CCAGGAACTGGGGAACACAAGGTCACAGTGAAAGTGGTGGCTGCCAATGACCTCAAGTGGCAGACTTCTG
GCATCTTCCGGCCGTTCATCGAGGTCAACATCATTGGGCCCCAGCTCAGCGACAAGAAACGCAAGTTTGC
GACCAAATCCAAGAACAATAGCTGGGCTCCCAAGTACAATGAGAGCTTCCAGTTCACGCTGAGCGCCGAC
GCGGGTCCCGAGTGCTATGAGCTGCAGGTGTGCGTCAAGGACTACTGCTTCGCGCGCGAGGACCGCACGG
TGGGGCTGGCCGTGCTGCAGCTGCGTGAGCTGGCCCAGCGCGGGAGCGCCGCCTGCTGGCTGCCGCTCGG
CCGCCGCATCCACATGGACGACACGGGCCTCACGGTGCTGCGAATCCTCTCGCAGCGCAGCAACGACGAG
GTGGCCAAGGAGTTCGTGAAGCTCAAGTCGGACACGCGCTCCGCCGAGGAGGGCGGTGCCGCGCCTGCGC
CTTAGCGCGGGCGGTCGGCCGAGCGGCACTGCGCCTGCGCGGAGGGCGCTGGGCGGGGAGGGACGGGGCT
TGCGCCTTGGTGGGACCTCCCCAGGGGCGGGGCTCGGGGGGCTCCACGCCAAGGGTGGGCTGCGCCTACG
CCCTTGACTCAGCTTTCCCTTTTGGGGAATTAGGAATGGAGGATGCCCCGCCCTCTCGGGAGGCCACGCC
CAAGGGCGCGACGAAGGAAGGAGCCACATCCCCAACTTGAGGCCACGCCCCCAGCACCTAGGGGGCATTT
TGAGCTGGGATGGGGGAAACCTCGTCCCTATGGAGGAGGCCACATCCCGGGGCTCTGGTACCGGGAGGCA
CCACCTCATGTCCCCTGGAAAAGCCATAAGATGGGACCCAGACCCCTGGGACCCCAGACCAATTGCCAAG
TATGGAAATCTCAGCTCCCTCGAGGGGGGGCCCTGGGCAAGGGGTAGGGCTCTCTGGAGCGCCCCTCTAG
GTGGCCTGGGGACTGGAGGGACCAGGATGCTGGTTGGAGGGCCCCGGAATACCGGAGTCCCTTTAGATAT
TTGTGCAAAAAATAAATGGGGGGAGGGGGGAGGATGGGATTTCAAAAGCACATGCGCCCTTGGGCGCCCA
AACCCTGGGGGCCGAGGGGACGGCTCTGGTTCCCCACGCTGCCCCTACTTCCCTTTGGGAGTTTGCCTCT
CCCTCTCCCCCAACAAACCCAGTCCTCATATCATAGAGTTCAACACACCCATTTGACAGATGGCAAAACT
GAGGCTTAAAGAGCTGCTTGAGACTTGGCCAAGGTTCCAGGTGCCATACCCTCTGTGCCCCTCCCTTAGG
CCTGTGTGCCCCATGGAAGGGTGGGCTGAGATCGGGATGACCTGACACAGCTCCCTATTGCTGCTAATTC
CCCCTCGGCCTCCTCCAAGGGGTGGGAATTCCAGGCCAAGACCCCTACTTCGCCTTTCCTTCTCCGGCTG
CCAAGCAGGACCTTTGCCCTCAGCCCTTTCTCCTGGGATCTCCATGGGGGATGCCATGAGGGCCTCCCAC
CACAAAAGAGAATTTGGGATCCCCTGGTCCCAGGTTTCTCCATCCCTTCTTCCTTTTCCAGAATTTTCCA
AATAGGAAAGAACAGAAGGAGACCAGAAACTCTAGGGGGGAGAAAGAGAATGAGAGAAAGAGAATGAGAG
AGAGAGAAACACAAACACAGTGACACAGTGAGAGCTTAGTCTCCAAGAGCCTATTCATTGATTCAAACAC
CCAAGCCACAGGATACCTCAGATGGCCCTCTTGCCAGCTGGAAGCTCTTTCTCCAATGAGCAAAGTTACA
GTGACCTGGCTGGAGTTACCTGGTGCACATAGGACCTTAGGGGAAAGTTCAGCGTGGACTACACTTGCTC
TGGGATCTGCTTTTCCACATGTGTGTATGGCACGCCTTTTTCTGCTGGATTGGGAAGGACAAGATTTTGC
TGTGCTAGGGAGAAATGAAAACGGGGTGAGCTGAGTAGCTGGGTTTCTGGAGGATAGAACATCAGATGGG
GAGGCTTTCCGAGGTGAAGAATGAGAGGGAACCACTTACTAGAGAGAAAAGAGCTCCAGGCCTGGGGAAC
AGCACGTGCGAAGGCCAGGAGAGAAGAACTGTTGAAACAACGAGAAGGGTGGCACGGCTGGAGCTGAGCC
AGCAAGGGGGATCGTGAGGAGCCTTGGGGTTGGGGAGATCTGCAGAAGCATCAGACCAGGCAGGGCCTCG
TACGCAGTCCTGAGGAGTTTTACTTTTATTCTAAGACAGTTGGGGAGCTCCAGGAGCTGTTTTAAGTTGG
GGAGAGACTGGATTCCAGCCTGCAAAAGCTGTTTTGTGAAGACTAAAACCAGTGAGGAGAGGTGGAGGTG
CTTTGGGGACACTGAAATGGATTCTTGGAAAGATTCTGAAGGCTGTGTTGAAAAGACACCTATAGCTGTG
GGGACATGACTATAATCCCAGCATTTGGGGAGACCGAGGCTGGCAGATCACTTAAGGTCAGGAGTTTGAG
ACCAGCCTGGCCAACATGGCGAAACCCCATCTCTGCTAAAAATACAAAAATTAGCTGGGTGCAGTGGTGC
ATGCCTGTAGTCCCAGCTACTCAGGAGACTGAGGCGGGAGAATTGTTTGAACCCTGGAGGCAGAGGTTGT
AGTGAGTCGTGATCACACAACTGCACTCCAGCCTGGGCAACAGAACAATACTCCATTCCCTCCCCTCTAC
CCCACCAAAAAAAAAAAAAAATCCTGCCCTTAGATGAGCTCTAGGGCTGCTGAGTACAGTTGTCCCAGTT
GCACAGTGCCCAAGGGTTTGGCATTGCTAAGAAGGCCACGTGCAAATCCTAGATATTGAGTGTTGTATGT
TTGTGACGTTGGTTTCCCGACATGTGAATGGCCCAAGTGTCTGGAAGAAGTGGCGCCACTTTCTAATTTG
CTTGGAGATGTTGCATGTCCCTTAAATTCAGACAGGTGCAGGTAACTGGAGGTTCTGAACCAAAGGTTAA
AATGCAAATTCTCATACAGGGTTGGGAAGTTGTAGCCAGGGATAAGCTTATGTGACTGTTATATGGACTG
AGGAGCAGATGTGAATTTCGAACCATGACATGGCTGAGGGTAGGGGTCGGGTGGATGGATGATTCAGGGT
TGTAACCCATAGAGCCCAAAGGGGAAGTGATCTGTGACCTGGGGTGAGGGTGATCTGGAAGATTTTTGGA
TGGCTGGAAAGAAATGGGGAAGTCGAGCTGCCTGAGAGAGCCAAGTTATTTCCCAAAAGATTCCTTAGGA
GTCTTTCTGTTCAAGACCTCCGTGTGTGTGTGTGTGTGTTTAGGGTTCCCCAGCAATGGCCCAGGCATGT
GAAGGAAACAAGCTTCTTCAGGGAATATTTGTTGAATGAGTTTTCCTGACTCCCAGGCTAGAACTGTTTT
TGCAATTTCCACCCTCTTTTCTTTCCCCCAGAGAACTCCTATTCGTCCTTCAAAACCCATCACGGAAACC
CCTCTTGGAGAAAACCCTCCTTCCTTCCCCTCAGGACTTTCCCAGCCACCGTCTCTCCTCCAGTCCAGCC
TGATGCCATGGGACTGGGGGTTTCTCTGTCCAGCTCTGTTTCTCCCAGACTGGGGTCTGAGGACTCTCAG
GACCCCCAACTTTACCTAGCACAGGCTGGGCACAAGTGGGTGACAGGGAGTCTACGCCTAGTGGAATTAT
GTATTGGGGCAGGGTCAGTGTGAGAATACACATCCGCATGCATGTCTGTCCATGTCTGTCCGTACCAACC
TTCCCCTTCCACACGGACCTGGGCACATAGGAGGTGTCTGAGCCTGACACATGGGACAGAGAGTGGACAT
GGCTGAGACACGGACAGAGAAAAGACAAGGAGTCCAGGGGGCTGAAAGCCTTTTGAAATCAGGAAGTTCC
TGTATTGGCAGAACAAAGCCCAGAGAGGAGCAGGGCTTTCCTCAACGCCACCCAGCAAGTGGACACAGAG
CCCGGCCTTGGATGACACCTCCAGGGTTCTGAACCCTGGACCTCGCTTTATGCAAGGAGCTGGCCCCACA
TTTCCATGAATCGGGGAAACAGCACAAGAAGGTTGGCCTGTGGCAGGGCAAGGGTTAAAGGGGTGACATT
GAGGGATGCCTCAGAGTCAAAGTCCCCTGACCAAGAGGAATAGAGTAGAAAACACAGAGACAGAGGGTGA
GATCACGCCCCGATGAGGACGGAGAGAGACAGAGATGGAGAGAGACATAGAGGTGGAAATATACAGAGAA
AGATAAATGCAGAGACCAAGGCAGGGAGTGTCGGGGGAAGTAAAGAGGGTGTCCTGAAGAAAGAAGGATC
TGTTCACTCTTACCAGTCTGTCCTCGAATGATTTGCATAAAATGAGGAGGTGCCTGTCCACACCCCCAAT
TCCTCTCTCAGGCCCCAGAGCCTGAGACCTCACCATGCCCCCATCAGAGATGCAAAAAACTAAACACCCA
ACTAGAAATCCTTGGGACCTCTCTCGGCTGGGATCTCAGAGCCTTTCTGTCCCCTACCCCTACCCCATGT
GCTGTCGATTTTGCAGATGGGGACAACCTGGGGCCTCCCGGAACTCTGCCACCCTGGGGAAGTTGGGGGA
GGGCCTTAGTCCCGGATCACAACCCCGTCTGCTCCCCAGAATCCTTTCCTAAGAATCGTTGAGGACCAAA
GTTGTCTTTGCTGACACGTGTTGCTTTTCTCTTTGCCTTTTATTGTTTCAGAGAAAAATCAAGTTGACTG
TGTCAAGTAACACCCCACCCCTTACCCCCGTCCAGCCATAGTGGCTCTCTGGAGACACAGGTCACAGGCG
GAGGGTCCCCTGATCATCCCCAACCACACAGCCAGGGGGACTTGACCCCTGTCCACCCCTGTCTCGTGCT
CCCTCAGACCCCCACAAACCGGCCAAGCAGTCCGGGGAGGCTTCCCCTCCACACAACTCTTAGCATGTGA
TTGCAGATGTGAAATCAAAACGTTGTTTGTTTTTTGTTTTGTTTTGATTCTACCCCGTCGGTCCAGTGTC
TGCACAGACGCCTTCATTTCTCTGTAAATATGTGACTTGGAACAAATGTTTAACACAAACGAGAAGTGGT
CATGAATGCATGGTGTTGAGATGTTTTGCACTATTCTGACTTTTTGGTCTCTGTAAAAATATTTTATTAA
CAGCAGACATTAAAAAAAGAAAAACCACACACA
(SEQ ID NO: 5058 (SOURCE NCBI Reference Sequence NM_001387021.1)

In various embodiments, an UNC13A mRNA transcript comprises the sequence provided as SEQ ID NO: 5059.

GCCCCCGGTGCTGAACCAAGATGGCCGGTGGCGGCCGGGCCCCGGCGTGAGCCAAGCGCGGGCTGCAGCC
GGGAGATGCCCCAGCCCAGCGGCCGCTGAGCCCGACCCGACAGAGCCGGCCCGGCCGCCTCCGGCCCACC
TGCGAGCTCGGAGACATGTCTCTGCTTTGCGTTGGAGTCAAAAAAGCCAAGTTTGATGGTGCCCAAGAGA
AATTCAACACGTACGTGACCCTGAAAGTGCAGAATGTCAAGAGCACGACCATCGCGGTGCGGGGCAGCCA
GCCCAGCTGGGAGCAGGATTTCATGTTCGAGATTAACCGTCTGGATTTGGGACTGACGGTGGAGGTGTGG
AATAAGGGTCTCATCTGGGACACAATGGTGGGCACTGTGTGGATCCCACTGAGGACCATCCGCCAGTCCA
ATGAGGAGGGCCCTGGAGAGTGGCTGACGCTGGACTCCCAGGTCATCATGGCAGACAGTGAGATCTGTGG
CACCAAGGACCCCACCTTCCACCGCATCCTCCTGGACACGCGCTTTGAGCTACCCTTAGACATTCCTGAA
GAGGAGGCTCGCTACTGGGCCAAGAAGCTGGAGCAGCTCAATGCTATGCGGGACCAGGATGAATATTCGT
TCCAAGATGAGCAAGACAAGCCTCTGCCTGTCCCCAGCAACCAGTGCTGCAACTGGAATTATTTTGGCTG
GGGTGAGCAGCACAACGATGACCCCGACAGTGCAGTGGATGATCGTGACAGTGACTACCGCAGTGAAACG
AGCAACAGCATCCCGCCGCCCTATTATACTACGTCACAACCCAACGCCTCAGTCCACCAATATTCTGTTC
GCCCACCACCCCTGGGCTCCCGGGAGTCCTACAGTGACTCCATGCACAGTTACGAGGAGTTCTCTGAGCC
ACAAGCCCTCAGCCCCACGGGTAGCAGCCGCTATGCCTCTTCCGGGGAGCTGAGCCAGGGAAGCTCTCAG
CTGAGCGAGGACTTCGACCCTGACGAGCACAGCCTGCAGGGCTCCGACATGGAGGATGAGCGGGACCGGG
ACTCCTACCACTCCTGCCACAGCTCGGTCAGCTACCACAAAGACTCGCCTCGCTGGGACCAGGATGAGGA
AGAGCTGGAGGAGGACCTGGAGGACTTCCTGGAGGAGGAGGAGCTGCCTGAAGATGAGGAGGAGCTGGAG
GAGGAGGAGGAGGAGGTGCCTGACGATTTGGGCAGCTATGCCCAGCGTGAAGACGTAGCTGTGGCTGAGC
CCAAAGACTTCAAACGCATCAGCCTCCCGCCAGCTGCCCCAGGGAAGGAGGACAAGGCCCCAGTGGCACC
CACCGAGGCCCCCGACATGGCCAAGGTGGCCCCCAAGCCAGCCACGCCCGACAAGGTGCCTGCAGCTGAG
CAGATCCCTGAGGCTGAGCCACCCAAGGACGAGGAGAGTTTCAGGCCGAGAGAGGATGAGGAAGGCCAGG
AGGGGCAGGACTCCATGTCCAGGGCCAAGGCCAACTGGCTGCGTGCCTTCAACAAGGTGCGGATGCAGCT
GCAGGAGGCCCGGGGAGAAGGAGAGATGTCTAAATCCCTATGGTTCAAAGGCGGCCCAGGGGGCGGTCTC
ATCATCATCGACAGCATGCCAGACATCCGCAAGAGGAAACCTATCCCACTCGTGAGCGACTTGTCCCTGG
TCCAGTCCAGGAAAGCGGGCATCACCTCGGCCTTGGCCTCCAGCACGTTGAACAACGAGGAGCTGAAAAA
CCACGTTTACAAGAAGACCCTGCAAGCCTTAATCTACCCCATCTCGTGCACGACGCCACACAACTTCGAA
GTGTGGACGGCCACCACGCCCACCTACTGCTACGAGTGCGAGGGGCTGCTGTGGGGCATCGCGAGGCAGG
GCATGCGCTGCACCGAGTGCGGTGTCAAGTGCCACGAGAAGTGCCAGGACCTGCTCAACGCCGACTGCCT
GCAGCGGGCTGCGGAGAAGAGCTCCAAGCACGGGGCGGAGGACCGGACACAGAACATCATCATGGTGCTC
AAGGACCGCATGAAGATCCGGGAGCGCAACAAGCCCGAGATCTTCGAGCTCATCCAGGAGATCTTCGCGG
TGACCAAGACGGCGCACACGCAGCAGATGAAGGCGGTCAAGCAGAGCGTGCTGGACGGCACGTCCAAGTG
GTCCGCCAAGATCAGCATCACCGTGGTCTGCGCCCAGGGCTTGCAGGCAAAGGACAAGACAGGATCCAGT
GACCCCTATGTCACCGTCCAGGTCGGGAAGACCAAGAAACGGACAAAAACCATCTATGGGAACCTCAACC
CGGTGTGGGAGGAGAATTTCCACTTTGAATGTCACAATTCCTCCGACCGCATCAAGGTGCGCGTCTGGGA
CGAGGATGACGACATCAAATCCCGCGTGAAACAGAGGTTCAAGAGGGAATCTGACGATTTCCTGGGGCAG
ACGATCATTGAGGTGCGGACGCTCAGCGGCGAGATGGACGTGTGGTACAACCTGGACAAGCGAACTGACA
AATCTGCCGTGTCGGGTGCCATCCGGCTCCACATCAGTGTGGAGATCAAAGGCGAGGAGAAGGTGGCCCC
GTACCATGTCCAGTACACCTGTCTGCATGAGAACCTGTTCCACTTCGTGACCGACGTGCAGAACAATGGG
GTCGTGAAGATCCCAGATGCCAAGGGTGACGATGCCTGGAAGGTTTACTACGATGAGACAGCCCAGGAGA
TTGTGGACGAGTTTGCCATGCGCTACGGCGTCGAGTCCATCTACCAAGCCATGACCCACTTTGCCTGCCT
CTCCTCCAAGTATATGTGCCCAGGGGTGCCTGCCGTCATGAGCACCCTGCTCGCCAACATCAATGCCTAC
TACGCACACACCACCGCCTCCACCAACGTGTCTGCCTCCGACCGCTTCGCCGCCTCCAACTTTGGGAAAG
AGCGCTTCGTGAAACTCCTGGACCAGCTGCATAACTCCCTGCGGATTGACCTCTCCATGTACCGGAATAA
CTTCCCAGCCAGCAGCCCGGAGAGACTCCAGGACCTCAAATCCACTGTGGACCTTCTCACCAGCATCACC
TTCTTTCGGATGAAGGTACAAGAACTCCAGAGCCCGCCCCGAGCCAGCCAGGTGGTAAAGGACTGTGTGA
AAGCCTGCCTTAATTCTACCTACGAGTACATCTTCAATAACTGCCATGAACTGTACAGCCGGGAGTACCA
GACAGACCCGGCCAAGAAGGGGGAAGTTCTCCCAGAGGAACAGGGGCCCAGCATCAAGAACCTCGACTTC
TGGTCCAAGCTGATTACCCTCATAGTGTCCATCATTGAGGAAGACAAGAATTCCTACACTCCCTGCCTCA
ACCAGTTTCCCCAGGAGCTGAATGTGGGTAAAATCAGCGCTGAAGTGATGTGGAATCTGTTTGCCCAAGA
CATGAAGTACGCCATGGAGGAGCACGACAAGCATCGTCTATGCAAGAGTGCCGACTACATGAACCTCCAC
TTCAAGGTGAAATGGCTCTACAATGAGTATGTGACGGAACTTCCCGCCTTCAAGGACCGCGTGCCTGAGT
ACCCTGCATGGTTTGAACCCTTCGTCATCCAGTGGCTGGATGAGAATGAGGAGGTGTCCCGGGATTTCCT
GCACGGTGCCCTGGAGCGAGACAAGAAGGATGGGTTCCAGCAGACCTCAGAGCATGCCCTATTCTCCTGC
TCCGTGGTGGATGTTTTCTCCCAACTCAACCAGAGCTTTGAAATCATCAAGAAACTCGAGTGTCCCGACC
CTCAGATCGTGGGGCACTACATGAGGCGCTTTGCCAAGACCATCAGTAATGTGCTCCTCCAGTATGCAGA
CATCATCTCCAAGGACTTTGCCTCCTACTGCTCCAAGGAGAAGGAGAAACCCTGCATTCTCATGAATAAC
ACTCAACAGCTACGAGTTCAGCTGGAGAAGATGTTCGAAGCCATGGGAGGAAAGGAGCTGGATGCTGAAG
CCAGTGACATCCTGAAGGAGCTTCAGGTGAAACTCAATAACGTCTTGGATGAGCTCAGCCGGGTGTTTGC
TACCAGCTTCCAGCCGCACATTGAAGAGTGTGTCAAACAGATGGGTGACATCCTTAGCCAGGTTAAGGGC
ACAGGCAATGTGCCAGCCAGTGCCTGCAGCAGCGTGGCCCAGGACGCGGACAATGTGTTGCAGCCCATCA
TGGACCTGCTGGACAGCAACCTGACCCTCTTTGCCAAAATCTGTGAGAAGACTGTGCTGAAGCGAGTGCT
GAAGGAGCTGTGGAAGCTGGTTATGAACACCATGGAGAAAACCATCGTCCTGCCGCCCCTCACTGACCAG
ACGGGGAACCTCTTGAGAAAACATGGCAAGGGATTAGAAAAGGGCAGGGTGAAATTGCCAAGCCACTCAG
ACGGAACCCAGATGATCTTCAATGCAGCCAAGGAGCTGGGTCAGCTGTCCAAACTCAAGGATCACATGGT
ACGAGAAGAAGCCAAGAGCTTGACCCCAAAGCAGTGCGCGGTTGTTGAGTTGGCCCTGGACACCATCAAG
CAATATTTCCACGCGGGTGGCGTGGGCCTCAAGAAGACCTTCCTGGAGAAGAGCCCGGACCTGCAATCCT
TGCGCTATGCCCTGTCGCTCTACACGCAGGCCACCGACCTGCTAATCAAGACCTTTGTACAGACGCAATC
GGCCCAGGGCTTGGGTGTAGAAGACCCTGTGGGTGAAGTCTCTGTCCATGTTGAGCTGTTCACTCATCCA
GGAACTGGGGAACACAAGGTCACAGTGAAAGTGGTGGCTGCCAATGACCTCAAGTGGCAGACTTCTGGCA
TCTTCCGGCCGTTCATCGAGGTCAACATCATTGGGCCCCAGCTCAGCGACAAGAAACGCAAGTTTGCGAC
CAAATCCAAGAACAATAGCTGGGCTCCCAAGTACAATGAGAGCTTCCAGTTCACGCTGAGCGCCGACGCG
GGTCCCGAGTGCTATGAGCTGCAGGTGTGCGTCAAGGACTACTGCTTCGCGCGCGAGGACCGCACGGTGG
GGCTGGCCGTGCTGCAGCTGCGTGAGCTGGCCCAGCGCGGGAGCGCCGCCTGCTGGCTGCCGCTCGGCCG
CCGCATCCACATGGACGACACGGGCCTCACGGTGCTGCGAATCCTCTCGCAGCGCAGCAACGACGAGGTG
GCCAAGGAGTTCGTGAAGCTCAAGTCGGACACGCGCTCCGCCGAGGAGGGCGGTGCCGCGCCTGCGCCTT
AGCGCGGGCGGTCGGCCGAGCGGCACTGCGCCTGCGCGGAGGGCGCTGGGCGGGGAGGGACGGGGCTTGC
GCCTTGGTGGGACCTCCCCAGGGGCGGGGCTCGGGGGGCTCCACGCCAAGGGTGGGCTGCGCCTACGCCC
TTGACTCAGCTTTCCCTTTTGGGGAATTAGGAATGGAGGATGCCCCGCCCTCTCGGGAGGCCACGCCCAA
GGGCGCGACGAAGGAAGGAGCCACATCCCCAACTTGAGGCCACGCCCCCAGCACCTAGGGGGCATTTTGA
GCTGGGATGGGGGAAACCTCGTCCCTATGGAGGAGGCCACATCCCGGGGCTCTGGTACCGGGAGGCACCA
CCTCATGTCCCCTGGAAAAGCCATAAGATGGGACCCAGACCCCTGGGACCCCAGACCAATTGCCAAGTAT
GGAAATCTCAGCTCCCTCGAGGGGGGGCCCTGGGCAAGGGGTAGGGCTCTCTGGAGCGCCCCTCTAGGTG
GCCTGGGGACTGGAGGGACCAGGATGCTGGTTGGAGGGCCCCGGAATACCGGAGTCCCTTTAGATATTTG
TGCAAAAAATAAATGGGGGGAGGGGGGAGGATGGGATTTCAAAAGCACATGCGCCCTTGGGCGCCCAAAC
CCTGGGGGCCGAGGGGACGGCTCTGGTTCCCCACGCTGCCCCTACTTCCCTTTGGGAGTTTGCCTCTCCC
TCTCCCCCAACAAACCCAGTCCTCATATCATAGAGTTCAACACACCCATTTGACAGATGGCAAAACTGAG
GCTTAAAGAGCTGCTTGAGACTTGGCCAAGGTTCCAGGTGCCATACCCTCTGTGCCCCTCCCTTAGGCCT
GTGTGCCCCATGGAAGGGTGGGCTGAGATCGGGATGACCTGACACAGCTCCCTATTGCTGCTAATTCCCC
CTCGGCCTCCTCCAAGGGGTGGGAATTCCAGGCCAAGACCCCTACTTCGCCTTTCCTTCTCCGGCTGCCA
AGCAGGACCTTTGCCCTCAGCCCTTTCTCCTGGGATCTCCATGGGGGATGCCATGAGGGCCTCCCACCAC
AAAAGAGAATTTGGGATCCCCTGGTCCCAGGTTTCTCCATCCCTTCTTCCTTTTCCAGAATTTTCCAAAT
AGGAAAGAACAGAAGGAGACCAGAAACTCTAGGGGGGAGAAAGAGAATGAGAGAAAGAGAATGAGAGAGA
GAGAAACACAAACACAGTGACACAGTGAGAGCTTAGTCTCCAAGAGCCTATTCATTGATTCAAACACCCA
AGCCACAGGATACCTCAGATGGCCCTCTTGCCAGCTGGAAGCTCTTTCTCCAATGAGCAAAGTTACAGTG
ACCTGGCTGGAGTTACCTGGTGCACATAGGACCTTAGGGGAAAGTTCAGCGTGGACTACACTTGCTCTGG
GATCTGCTTTTCCACATGTGTGTATGGCACGCCTTTTTCTGCTGGATTGGGAAGGACAAGATTTTGCTGT
GCTAGGGAGAAATGAAAACGGGGTGAGCTGAGTAGCTGGGTTTCTGGAGGATAGAACATCAGATGGGGAG
GCTTTCCGAGGTGAAGAATGAGAGGGAACCACTTACTAGAGAGAAAAGAGCTCCAGGCCTGGGGAACAGC
ACGTGCGAAGGCCAGGAGAGAAGAACTGTTGAAACAACGAGAAGGGTGGCACGGCTGGAGCTGAGCCAGC
AAGGGGGATCGTGAGGAGCCTTGGGGTTGGGGAGATCTGCAGAAGCATCAGACCAGGCAGGGCCTCGTAC
GCAGTCCTGAGGAGTTTTACTTTTATTCTAAGACAGTTGGGGAGCTCCAGGAGCTGTTTTAAGTTGGGGA
GAGACTGGATTCCAGCCTGCAAAAGCTGTTTTGTGAAGACTAAAACCAGTGAGGAGAGGTGGAGGTGCTT
TGGGGACACTGAAATGGATTCTTGGAAAGATTCTGAAGGCTGTGTTGAAAAGACACCTATAGCTGTGGGG
ACATGACTATAATCCCAGCATTTGGGGAGACCGAGGCTGGCAGATCACTTAAGGTCAGGAGTTTGAGACC
AGCCTGGCCAACATGGCGAAACCCCATCTCTGCTAAAAATACAAAAATTAGCTGGGTGCAGTGGTGCATG
CCTGTAGTCCCAGCTACTCAGGAGACTGAGGCGGGAGAATTGTTTGAACCCTGGAGGCAGAGGTTGTAGT
GAGTCGTGATCACACAACTGCACTCCAGCCTGGGCAACAGAACAATACTCCATTCCCTCCCCTCTACCCC
ACCAAAAAAAAAAAAAAATCCTGCCCTTAGATGAGCTCTAGGGCTGCTGAGTACAGTTGTCCCAGTTGCA
CAGTGCCCAAGGGTTTGGCATTGCTAAGAAGGCCACGTGCAAATCCTAGATATTGAGTGTTGTATGTTTG
TGACGTTGGTTTCCCGACATGTGAATGGCCCAAGTGTCTGGAAGAAGTGGCGCCACTTTCTAATTTGCTT
GGAGATGTTGCATGTCCCTTAAATTCAGACAGGTGCAGGTAACTGGAGGTTCTGAACCAAAGGTTAAAAT
GCAAATTCTCATACAGGGTTGGGAAGTTGTAGCCAGGGATAAGCTTATGTGACTGTTATATGGACTGAGG
AGCAGATGTGAATTTCGAACCATGACATGGCTGAGGGTAGGGGTCGGGTGGATGGATGATTCAGGGTTGT
AACCCATAGAGCCCAAAGGGGAAGTGATCTGTGACCTGGGGTGAGGGTGATCTGGAAGATTTTTGGATGG
CTGGAAAGAAATGGGGAAGTCGAGCTGCCTGAGAGAGCCAAGTTATTTCCCAAAAGATTCCTTAGGAGTC
TTTCTGTTCAAGACCTCCGTGTGTGTGTGTGTGTGTTTAGGGTTCCCCAGCAATGGCCCAGGCATGTGAA
GGAAACAAGCTTCTTCAGGGAATATTTGTTGAATGAGTTTTCCTGACTCCCAGGCTAGAACTGTTTTTGC
AATTTCCACCCTCTTTTCTTTCCCCCAGAGAACTCCTATTCGTCCTTCAAAACCCATCACGGAAACCCCT
CTTGGAGAAAACCCTCCTTCCTTCCCCTCAGGACTTTCCCAGCCACCGTCTCTCCTCCAGTCCAGCCTGA
TGCCATGGGACTGGGGGTTTCTCTGTCCAGCTCTGTTTCTCCCAGACTGGGGTCTGAGGACTCTCAGGAC
CCCCAACTTTACCTAGCACAGGCTGGGCACAAGTGGGTGACAGGGAGTCTACGCCTAGTGGAATTATGTA
TTGGGGCAGGGTCAGTGTGAGAATACACATCCGCATGCATGTCTGTCCATGTCTGTCCGTACCAACCTTC
CCCTTCCACACGGACCTGGGCACATAGGAGGTGTCTGAGCCTGACACATGGGACAGAGAGTGGACATGGC
TGAGACACGGACAGAGAAAAGACAAGGAGTCCAGGGGGCTGAAAGCCTTTTGAAATCAGGAAGTTCCTGT
ATTGGCAGAACAAAGCCCAGAGAGGAGCAGGGCTTTCCTCAACGCCACCCAGCAAGTGGACACAGAGCCC
GGCCTTGGATGACACCTCCAGGGTTCTGAACCCTGGACCTCGCTTTATGCAAGGAGCTGGCCCCACATTT
CCATGAATCGGGGAAACAGCACAAGAAGGTTGGCCTGTGGCAGGGCAAGGGTTAAAGGGGTGACATTGAG
GGATGCCTCAGAGTCAAAGTCCCCTGACCAAGAGGAATAGAGTAGAAAACACAGAGACAGAGGGTGAGAT
CACGCCCCGATGAGGACGGAGAGAGACAGAGATGGAGAGAGACATAGAGGTGGAAATATACAGAGAAAGA
TAAATGCAGAGACCAAGGCAGGGAGTGTCGGGGGAAGTAAAGAGGGTGTCCTGAAGAAAGAAGGATCTGT
TCACTCTTACCAGTCTGTCCTCGAATGATTTGCATAAAATGAGGAGGTGCCTGTCCACACCCCCAATTCC
TCTCTCAGGCCCCAGAGCCTGAGACCTCACCATGCCCCCATCAGAGATGCAAAAAACTAAACACCCAACT
AGAAATCCTTGGGACCTCTCTCGGCTGGGATCTCAGAGCCTTTCTGTCCCCTACCCCTACCCCATGTGCT
GTCGATTTTGCAGATGGGGACAACCTGGGGCCTCCCGGAACTCTGCCACCCTGGGGAAGTTGGGGGAGGG
CCTTAGTCCCGGATCACAACCCCGTCTGCTCCCCAGAATCCTTTCCTAAGAATCGTTGAGGACCAAAGTT
GTCTTTGCTGACACGTGTTGCTTTTCTCTTTGCCTTTTATTGTTTCAGAGAAAAATCAAGTTGACTGTGT
CAAGTAACACCCCACCCCTTACCCCCGTCCAGCCATAGTGGCTCTCTGGAGACACAGGTCACAGGCGGAG
GGTCCCCTGATCATCCCCAACCACACAGCCAGGGGGACTTGACCCCTGTCCACCCCTGTCTCGTGCTCCC
TCAGACCCCCACAAACCGGCCAAGCAGTCCGGGGAGGCTTCCCCTCCACACAACTCTTAGCATGTGATTG
CAGATGTGAAATCAAAACGTTGTTTGTTTTTTGTTTTGTTTTGATTCTACCCCGTCGGTCCAGTGTCTGC
ACAGACGCCTTCATTTCTCTGTAAATATGTGACTTGGAACAAATGTTTAACACAAACGAGAAGTGGTCAT
GAATGCATGGTGTTGAGATGTTTTGCACTATTCTGACTTTTTGGTCTCTGTAAAAATATTTTATTAACAG
CAGACATTAAAAAAAGAAAAACCACACACA
(SEQ ID NO: 5059 (SOURCE NCBI Reference Sequence NM_001387022.1)).

In various embodiments, an UNC13A mRNA transcript comprises the sequence provided as SEQ ID NO: 5060.

GCCCCCGGTGCTGAACCAAGATGGCCGGTGGCGGCCGGGCCCCGGCGTGAGCCAAGCGCGGGCTGCAGCC
GGGAGATGCCCCAGCCCAGCGGCCGCTGAGCCCGACCCGACAGAGCCGGCCCGGCCGCCTCCGGCCCACC
TGCGAGCTCGGAGACATGTCTCTGCTTTGCGTTGGAGTCAAAAAAGCCAAGTTTGATGGTGCCCAAGAGA
AATTCAACACGTACGTGACCCTGAAAGTGCAGAATGTCAAGAGCACGACCATCGCGGTGCGGGGCAGCCA
GCCCAGCTGGGAGCAGGATTTCATGTTCGAGATTAACCGTCTGGATTTGGGACTGACGGTGGAGGTGTGG
AATAAGGGTCTCATCTGGGACACAATGGTGGGCACTGTGTGGATCCCACTGAGGACCATCCGCCAGTCCA
ATGAGGAGGGCCCTGGAGAGTGGCTGACGCTGGACTCCCAGGTCATCATGGCAGACAGTGAGATCTGTGG
CACCAAGGACCCCACCTTCCACCGCATCCTCCTGGACACGCGCTTTGAGCTACCCTTAGACATTCCTGAA
GAGGAGGCTCGCTACTGGGCCAAGAAGCTGGAGCAGCTCAATGCTATGCGGGACCAGGATGAATATTCGT
TCCAAGATGAGCAAGACAAGCCTCTGCCTGTCCCCAGCAACCAGTGCTGCAACTGGAATTATTTTGGCTG
GGGTGAGCAGCACAACGATGACCCCGACAGTGCAGTGGATGATCGTGACAGTGACTACCGCAGTGAAACG
AGCAACAGCATCCCGCCGCCCTATTATACTACGTCACAACCCAACGCCTCAGTCCACCAATATTCTGTTC
GCCCACCACCCCTGGGCTCCCGGGAGTCCTACAGTGACTCCATGCACAGTTACGAGGAGTTCTCTGAGCC
ACAAGCCCTCAGCCCCACGGGTAGCAGCCGCTATGCCTCTTCCGGGGAGCTGAGCCAGGGAAGCTCTCAG
CTGAGCGAGGACTTCGACCCTGACGAGCACAGCCTGCAGGGCTCCGACATGGAGGATGAGCGGGACCGGG
ACTCCTACCACTCCTGCCACAGCTCGGTCAGCTACCACAAAGACTCGCCTCGCTGGGACCAGGATGAGGA
AGAGCTGGAGGAGGACCTGGAGGACTTCCTGGAGGAGGAGGAGCTGCCTGAAGATGAGGAGGAGCTGGAG
GAGGAGGAGGAGGAGGTGCCTGACGATTTGGGCAGCTATGCCCAGCGTGAAGACGTAGCTGTGGCTGAGC
CCAAAGACTTCAAACGCATCAGCCTCCCGCCAGCTGCCCCAGGGAAGGAGGACAAGGCCCCAGTGGCACC
CACCGAGGCCCCCGACATGGCCAAGGTGGCCCCCAAGCCAGCCACGCCCGACAAGGTGCCTGCAGCTGAG
CAGATCCCTGAGGCTGAGCCACCCAAGGACGAGGAGAGTTTCAGGCCGAGAGAGGATGAGGAAGGCCAGG
AGGGGCAGGACTCCATGTCCAGGGCCAAGGCCAACTGGCTGCGTGCCTTCAACAAGGTGCGGATGCAGCT
GCAGGAGGCCCGGGGAGAAGGAGAGATGTCTAAATCCCTATGGTTCAAAGGCGGCCCAGGGGGCGGTCTC
ATCATCATCGACAGCATGCCAGACATCCGCAAGAGGAAACCTATCCCACTCGTGAGCGACTTGTCCCTGG
TCCAGTCCAGGAAAGCGGGCATCACCTCGGCCTTGGCCTCCAGCACGTTGAACAACGAGGAGCTGAAAAA
CCACGTTTACAAGAAGACCCTGCAAGCCTTAATCTACCCCATCTCGTGCACGACGCCACACAACTTCGAA
GTGTGGACGGCCACCACGCCCACCTACTGCTACGAGTGCGAGGGGCTGCTGTGGGGCATCGCGAGGCAGG
GCATGCGCTGCACCGAGTGCGGTGTCAAGTGCCACGAGAAGTGCCAGGACCTGCTCAACGCCGACTGCCT
GCAGCGGGCTGCGGAGAAGAGCTCCAAGCACGGGGCGGAGGACCGGACACAGAACATCATCATGGTGCTC
AAGGACCGCATGAAGATCCGGGAGCGCAACAAGCCCGAGATCTTCGAGCTCATCCAGGAGATCTTCGCGG
TGACCAAGACGGCGCACACGCAGCAGATGAAGGCGGTCAAGCAGAGCGTGCTGGACGGCACGTCCAAGTG
GTCCGCCAAGATCAGCATCACCGTGGTCTGCGCCCAGGGCTTGCAGGCAAAGGACAAGACAGGATCCAGT
GACCCCTATGTCACCGTCCAGGTCGGGAAGACCAAGAAACGGACAAAAACCATCTATGGGAACCTCAACC
CGGTGTGGGAGGAGAATTTCCACTTTGAATGTCACAATTCCTCCGACCGCATCAAGGTGCGCGTCTGGGA
CGAGGATGACGACATCAAATCCCGCGTGAAACAGAGGTTCAAGAGGGAATCTGACGATTTCCTGGGGCAG
ACGATCATTGAGGTGCGGACGCTCAGCGGCGAGATGGACGTGTGGTACAACCTGGACAAGCGAACTGACA
AATCTGCCGTGTCGGGTGCCATCCGGCTCCACATCAGTGTGGAGATCAAAGGCGAGGAGAAGGTGGCCCC
GTACCATGTCCAGTACACCTGTCTGCATGAGAACCTGTTCCACTTCGTGACCGACGTGCAGAACAATGGG
GTCGTGAAGATCCCAGATGCCAAGGGTGACGATGCCTGGAAGGTTTACTACGATGAGACAGCCCAGGAGA
TTGTGGACGAGTTTGCCATGCGCTACGGCGTCGAGTCCATCTACCAAGCCATGACCCACTTTGCCTGCCT
CTCCTCCAAGTATATGTGCCCAGGGGTGCCTGCCGTCATGAGCACCCTGCTCGCCAACATCAATGCCTAC
TACGCACACACCACCGCCTCCACCAACGTGTCTGCCTCCGACCGCTTCGCCGCCTCCAACTTTGGGAAAG
AGCGCTTCGTGAAACTCCTGGACCAGCTGCATAACTCCCTGCGGATTGACCTCTCCATGTACCGGAATAA
CTTCCCAGCCAGCAGCCCGGAGAGACTCCAGGACCTCAAATCCACTGTGGACCTTCTCACCAGCATCACC
TTCTTTCGGATGAAGGTACAAGAACTCCAGAGCCCGCCCCGAGCCAGCCAGGTGGTAAAGGACTGTGTGA
AAGCCTGCCTTAATTCTACCTACGAGTACATCTTCAATAACTGCCATGAACTGTACAGCCGGGAGTACCA
GACAGACCCGGCCAAGAAGGGGGAAGTTCTCCCAGAGGAACAGGGGCCCAGCATCAAGAACCTCGACTTC
TGGTCCAAGCTGATTACCCTCATAGTGTCCATCATTGAGGAAGACAAGAATTCCTACACTCCCTGCCTCA
ACCAGTTTCCCCAGGAGCTGAATGTGGGTAAAATCAGCGCTGAAGTGATGTGGAATCTGTTTGCCCAAGA
CATGAAGTACGCCATGGAGGAGCACGACAAGCATCGTCTATGCAAGAGTGCCGACTACATGAACCTCCAC
TTCAAGGTGAAATGGCTCTACAATGAGTATGTGACGGAACTTCCCGCCTTCAAGGACCGCGTGCCTGAGT
ACCCTGCATGGTTTGAACCCTTCGTCATCCAGTGGCTGGATGAGAATGAGGAGGTGTCCCGGGATTTCCT
GCACGGTGCCCTGGAGCGAGACAAGAAGGATGGGTTCCAGCAGACCTCAGAGCATGCCCTATTCTCCTGC
TCCGTGGTGGATGTTTTCTCCCAACTCAACCAGAGCTTTGAAATCATCAAGAAACTCGAGTGTCCCGACC
CTCAGATCGTGGGGCACTACATGAGGCGCTTTGCCAAGACCATCAGTAATGTGCTCCTCCAGTATGCAGA
CATCATCTCCAAGGACTTTGCCTCCTACTGCTCCAAGGAGAAGGAGAAAGTGCCCTGCATTCTCATGAAT
AACACTCAACAGCTACGAGTTCAGCTGGAGAAGATGTTCGAAGCCATGGGAGGAAAGGAGCTGGATGCTG
AAGCCAGTGACATCCTGAAGGAGCTTCAGGTGAAACTCAATAACGTCTTGGATGAGCTCAGCCGGGTGTT
TGCTACCAGCTTCCAGCCGCACATTGAAGAGTGTGTCAAACAGATGGGTGACATCCTTAGCCAGGTTAAG
GGCACAGGCAATGTGCCAGCCAGTGCCTGCAGCAGCGTGGCCCAGGACGCGGACAATGTGTTGCAGCCCA
TCATGGACCTGCTGGACAGCAACCTGACCCTCTTTGCCAAAATCTGTGAGAAGACTGTGCTGAAGCGAGT
GCTGAAGGAGCTGTGGAAGCTGGTTATGAACACCATGGAGAAAACCATCGTCCTGCCGCCCCTCACTGAC
CAGACGGGAACCCAGATGATCTTCAATGCAGCCAAGGAGCTGGGTCAGCTGTCCAAACTCAAGGATCACA
TGGTACGAGAAGAAGCCAAGAGCTTGACCCCAAAGCAGTGCGCGGTTGTTGAGTTGGCCCTGGACACCAT
CAAGCAATATTTCCACGCGGGTGGCGTGGGCCTCAAGAAGACCTTCCTGGAGAAGAGCCCGGACCTGCAA
TCCTTGCGCTATGCCCTGTCGCTCTACACGCAGGCCACCGACCTGCTAATCAAGACCTTTGTACAGACGC
AATCGGCCCAGGGCTTGGGTGTAGAAGACCCTGTGGGTGAAGTCTCTGTCCATGTTGAGCTGTTCACTCA
TCCAGGAACTGGGGAACACAAGGTCACAGTGAAAGTGGTGGCTGCCAATGACCTCAAGTGGCAGACTTCT
GGCATCTTCCGGCCGTTCATCGAGGTCAACATCATTGGGCCCCAGCTCAGCGACAAGAAACGCAAGTTTG
CGACCAAATCCAAGAACAATAGCTGGGCTCCCAAGTACAATGAGAGCTTCCAGTTCACGCTGAGCGCCGA
CGCGGGTCCCGAGTGCTATGAGCTGCAGGTGTGCGTCAAGGACTACTGCTTCGCGCGCGAGGACCGCACG
GTGGGGCTGGCCGTGCTGCAGCTGCGTGAGCTGGCCCAGCGCGGGAGCGCCGCCTGCTGGCTGCCGCTCG
GCCGCCGCATCCACATGGACGACACGGGCCTCACGGTGCTGCGAATCCTCTCGCAGCGCAGCAACGACGA
GGTGGCCAAGGAGTTCGTGAAGCTCAAGTCGGACACGCGCTCCGCCGAGGAGGGCGGTGCCGCGCCTGCG
CCTTAGCGCGGGCGGTCGGCCGAGCGGCACTGCGCCTGCGCGGAGGGCGCTGGGCGGGGAGGGACGGGGC
TTGCGCCTTGGTGGGACCTCCCCAGGGGCGGGGCTCGGGGGGCTCCACGCCAAGGGTGGGCTGCGCCTAC
GCCCTTGACTCAGCTTTCCCTTTTGGGGAATTAGGAATGGAGGATGCCCCGCCCTCTCGGGAGGCCACGC
CCAAGGGCGCGACGAAGGAAGGAGCCACATCCCCAACTTGAGGCCACGCCCCCAGCACCTAGGGGGCATT
TTGAGCTGGGATGGGGGAAACCTCGTCCCTATGGAGGAGGCCACATCCCGGGGCTCTGGTACCGGGAGGC
ACCACCTCATGTCCCCTGGAAAAGCCATAAGATGGGACCCAGACCCCTGGGACCCCAGACCAATTGCCAA
GTATGGAAATCTCAGCTCCCTCGAGGGGGGGCCCTGGGCAAGGGGTAGGGCTCTCTGGAGCGCCCCTCTA
GGTGGCCTGGGGACTGGAGGGACCAGGATGCTGGTTGGAGGGCCCCGGAATACCGGAGTCCCTTTAGATA
TTTGTGCAAAAAATAAATGGGGGGAGGGGGGAGGATGGGATTTCAAAAGCACATGCGCCCTTGGGCGCCC
AAACCCTGGGGGCCGAGGGGACGGCTCTGGTTCCCCACGCTGCCCCTACTTCCCTTTGGGAGTTTGCCTC
TCCCTCTCCCCCAACAAACCCAGTCCTCATATCATAGAGTTCAACACACCCATTTGACAGATGGCAAAAC
TGAGGCTTAAAGAGCTGCTTGAGACTTGGCCAAGGTTCCAGGTGCCATACCCTCTGTGCCCCTCCCTTAG
GCCTGTGTGCCCCATGGAAGGGTGGGCTGAGATCGGGATGACCTGACACAGCTCCCTATTGCTGCTAATT
CCCCCTCGGCCTCCTCCAAGGGGTGGGAATTCCAGGCCAAGACCCCTACTTCGCCTTTCCTTCTCCGGCT
GCCAAGCAGGACCTTTGCCCTCAGCCCTTTCTCCTGGGATCTCCATGGGGGATGCCATGAGGGCCTCCCA
CCACAAAAGAGAATTTGGGATCCCCTGGTCCCAGGTTTCTCCATCCCTTCTTCCTTTTCCAGAATTTTCC
AAATAGGAAAGAACAGAAGGAGACCAGAAACTCTAGGGGGGAGAAAGAGAATGAGAGAAAGAGAATGAGA
GAGAGAGAAACACAAACACAGTGACACAGTGAGAGCTTAGTCTCCAAGAGCCTATTCATTGATTCAAACA
CCCAAGCCACAGGATACCTCAGATGGCCCTCTTGCCAGCTGGAAGCTCTTTCTCCAATGAGCAAAGTTAC
AGTGACCTGGCTGGAGTTACCTGGTGCACATAGGACCTTAGGGGAAAGTTCAGCGTGGACTACACTTGCT
CTGGGATCTGCTTTTCCACATGTGTGTATGGCACGCCTTTTTCTGCTGGATTGGGAAGGACAAGATTTTG
CTGTGCTAGGGAGAAATGAAAACGGGGTGAGCTGAGTAGCTGGGTTTCTGGAGGATAGAACATCAGATGG
GGAGGCTTTCCGAGGTGAAGAATGAGAGGGAACCACTTACTAGAGAGAAAAGAGCTCCAGGCCTGGGGAA
CAGCACGTGCGAAGGCCAGGAGAGAAGAACTGTTGAAACAACGAGAAGGGTGGCACGGCTGGAGCTGAGC
CAGCAAGGGGGATCGTGAGGAGCCTTGGGGTTGGGGAGATCTGCAGAAGCATCAGACCAGGCAGGGCCTC
GTACGCAGTCCTGAGGAGTTTTACTTTTATTCTAAGACAGTTGGGGAGCTCCAGGAGCTGTTTTAAGTTG
GGGAGAGACTGGATTCCAGCCTGCAAAAGCTGTTTTGTGAAGACTAAAACCAGTGAGGAGAGGTGGAGGT
GCTTTGGGGACACTGAAATGGATTCTTGGAAAGATTCTGAAGGCTGTGTTGAAAAGACACCTATAGCTGT
GGGGACATGACTATAATCCCAGCATTTGGGGAGACCGAGGCTGGCAGATCACTTAAGGTCAGGAGTTTGA
GACCAGCCTGGCCAACATGGCGAAACCCCATCTCTGCTAAAAATACAAAAATTAGCTGGGTGCAGTGGTG
CATGCCTGTAGTCCCAGCTACTCAGGAGACTGAGGCGGGAGAATTGTTTGAACCCTGGAGGCAGAGGTTG
TAGTGAGTCGTGATCACACAACTGCACTCCAGCCTGGGCAACAGAACAATACTCCATTCCCTCCCCTCTA
CCCCACCAAAAAAAAAAAAAAATCCTGCCCTTAGATGAGCTCTAGGGCTGCTGAGTACAGTTGTCCCAGT
TGCACAGTGCCCAAGGGTTTGGCATTGCTAAGAAGGCCACGTGCAAATCCTAGATATTGAGTGTTGTATG
TTTGTGACGTTGGTTTCCCGACATGTGAATGGCCCAAGTGTCTGGAAGAAGTGGCGCCACTTTCTAATTT
GCTTGGAGATGTTGCATGTCCCTTAAATTCAGACAGGTGCAGGTAACTGGAGGTTCTGAACCAAAGGTTA
AAATGCAAATTCTCATACAGGGTTGGGAAGTTGTAGCCAGGGATAAGCTTATGTGACTGTTATATGGACT
GAGGAGCAGATGTGAATTTCGAACCATGACATGGCTGAGGGTAGGGGTCGGGTGGATGGATGATTCAGGG
TTGTAACCCATAGAGCCCAAAGGGGAAGTGATCTGTGACCTGGGGTGAGGGTGATCTGGAAGATTTTTGG
ATGGCTGGAAAGAAATGGGGAAGTCGAGCTGCCTGAGAGAGCCAAGTTATTTCCCAAAAGATTCCTTAGG
AGTCTTTCTGTTCAAGACCTCCGTGTGTGTGTGTGTGTGTTTAGGGTTCCCCAGCAATGGCCCAGGCATG
TGAAGGAAACAAGCTTCTTCAGGGAATATTTGTTGAATGAGTTTTCCTGACTCCCAGGCTAGAACTGTTT
TTGCAATTTCCACCCTCTTTTCTTTCCCCCAGAGAACTCCTATTCGTCCTTCAAAACCCATCACGGAAAC
CCCTCTTGGAGAAAACCCTCCTTCCTTCCCCTCAGGACTTTCCCAGCCACCGTCTCTCCTCCAGTCCAGC
CTGATGCCATGGGACTGGGGGTTTCTCTGTCCAGCTCTGTTTCTCCCAGACTGGGGTCTGAGGACTCTCA
GGACCCCCAACTTTACCTAGCACAGGCTGGGCACAAGTGGGTGACAGGGAGTCTACGCCTAGTGGAATTA
TGTATTGGGGCAGGGTCAGTGTGAGAATACACATCCGCATGCATGTCTGTCCATGTCTGTCCGTACCAAC
CTTCCCCTTCCACACGGACCTGGGCACATAGGAGGTGTCTGAGCCTGACACATGGGACAGAGAGTGGACA
TGGCTGAGACACGGACAGAGAAAAGACAAGGAGTCCAGGGGGCTGAAAGCCTTTTGAAATCAGGAAGTTC
CTGTATTGGCAGAACAAAGCCCAGAGAGGAGCAGGGCTTTCCTCAACGCCACCCAGCAAGTGGACACAGA
GCCCGGCCTTGGATGACACCTCCAGGGTTCTGAACCCTGGACCTCGCTTTATGCAAGGAGCTGGCCCCAC
ATTTCCATGAATCGGGGAAACAGCACAAGAAGGTTGGCCTGTGGCAGGGCAAGGGTTAAAGGGGTGACAT
TGAGGGATGCCTCAGAGTCAAAGTCCCCTGACCAAGAGGAATAGAGTAGAAAACACAGAGACAGAGGGTG
AGATCACGCCCCGATGAGGACGGAGAGAGACAGAGATGGAGAGAGACATAGAGGTGGAAATATACAGAGA
AAGATAAATGCAGAGACCAAGGCAGGGAGTGTCGGGGGAAGTAAAGAGGGTGTCCTGAAGAAAGAAGGAT
CTGTTCACTCTTACCAGTCTGTCCTCGAATGATTTGCATAAAATGAGGAGGTGCCTGTCCACACCCCCAA
TTCCTCTCTCAGGCCCCAGAGCCTGAGACCTCACCATGCCCCCATCAGAGATGCAAAAAACTAAACACCC
AACTAGAAATCCTTGGGACCTCTCTCGGCTGGGATCTCAGAGCCTTTCTGTCCCCTACCCCTACCCCATG
TGCTGTCGATTTTGCAGATGGGGACAACCTGGGGCCTCCCGGAACTCTGCCACCCTGGGGAAGTTGGGGG
AGGGCCTTAGTCCCGGATCACAACCCCGTCTGCTCCCCAGAATCCTTTCCTAAGAATCGTTGAGGACCAA
AGTTGTCTTTGCTGACACGTGTTGCTTTTCTCTTTGCCTTTTATTGTTTCAGAGAAAAATCAAGTTGACT
GTGTCAAGTAACACCCCACCCCTTACCCCCGTCCAGCCATAGTGGCTCTCTGGAGACACAGGTCACAGGC
GGAGGGTCCCCTGATCATCCCCAACCACACAGCCAGGGGGACTTGACCCCTGTCCACCCCTGTCTCGTGC
TCCCTCAGACCCCCACAAACCGGCCAAGCAGTCCGGGGAGGCTTCCCCTCCACACAACTCTTAGCATGTG
ATTGCAGATGTGAAATCAAAACGTTGTTTGTTTTTTGTTTTGTTTTGATTCTACCCCGTCGGTCCAGTGT
CTGCACAGACGCCTTCATTTCTCTGTAAATATGTGACTTGGAACAAATGTTTAACACAAACGAGAAGTGG
TCATGAATGCATGGTGTTGAGATGTTTTGCACTATTCTGACTTTTTGGTCTCTGTAAAAATATTTTATTA
ACAGCAGACATTAAAAAAAGAAAAACCACACACA
(SEQ ID NO: 5060 (SOURCE NCBI Reference Sequence NM_001387023.1)

In various embodiments, an UNC13A mRNA transcript comprises the sequence provided as SEQ ID NO: 5061.

CTGAACCAAGATGGCCGGTGGCGGCCGGGCCCCGGCGTGAGCCAAGCGCGGGCTGCAGCCGGGAGATGCC
CCAGCCCAGCGGCCGCTGAGCCCGACCCGACAGAGCCGGCCCGGCCGCCTCCGGCCCACCTGCGAGCTCG
GAGACATGTCTCTGCTTTGCGTTGGAGAGAAATTCAACACGTACGTGACCCTGAAAGTGCAGAATGTCAA
GAGCACGACCATCGCGGTGCGGGGCAGCCAGCCCAGCTGGGAGCAGGATTTCATGTTCGAGATTAACCGT
CTGGATTTGGGACTGACGGTGGAGGTGTGGAATAAGGGTCTCATCTGGGACACAATGGTGGGCACTGTGT
GGATCCCACTGAGGACCATCCGCCAGTCCAATGAGGAGGGCCCTGGAGAGTGGCTGACGCTGGACTCCCA
GGTCATCATGGCAGACAGTGAGATCTGTGGCACCAAGGACCCCACCTTCCACCGCATCCTCCTGGACACG
CGCTTTGAGCTACCCTTAGACATTCCTGAAGAGGAGGCTCGCTACTGGGCCAAGAAGCTGGAGCAGCTCA
ATGCTATGCGGGACCAGGATGAATATTCGTTCCAAGATGAGCAAGACAAGCCTCTGCCTGTCCCCAGCAA
CCAGTGCTGCAACTGGAATTATTTTGGCTGGGGTGAGCAGCACAACGATGACCCCGACAGTGCAGTGGAT
GATCGTGACAGTGACTACCGCAGTGAAACGAGCAACAGCATCCCGCCGCCCTATTATACTACGTCACAAC
CCAACGCCTCAGTCCACCAATATTCTGTTCGCCCACCACCCCTGGGCTCCCGGGAGTCCTACAGTGACTC
CATGCACAGTTACGAGGAGTTCTCTGAGCCACAAGCCCTCAGCCCCACGGGTAGCAGCCGCTATGCCTCT
TCCGGGGAGCTGAGCCAGGGAAGCTCTCAGCTGAGCGAGGACTTCGACCCTGACGAGCACAGCCTGCAGG
GCTCCGACATGGAGGATGAGCGGGACCGGGACTCCTACCACTCCTGCCACAGCTCGGTCAGCTACCACAA
AGACTCGCCTCGCTGGGACCAGGATGAGGAAGAGCTGGAGGAGGACCTGGAGGACTTCCTGGAGGAGGAG
GAGCTGCCTGAAGATGAGGAGGAGCTGGAGGAGGAGGAGGAGGAGGTGCCTGACGATTTGGGCAGCTATG
CCCAGCGTGAAGACGTAGCTGTGGCTGAGCCCAAAGACTTCAAACGCATCAGCCTCCCGCCAGCTGCCCC
AGGGAAGGAGGACAAGGCCCCAGTGGCACCCACCGAGGCCCCCGACATGGCCAAGGTGGCCCCCAAGCCA
GCCACGCCCGACAAGGTGCCTGCAGCTGAGCAGATCCCTGAGGCTGAGCCACCCAAGGACGAGGAGAGTT
TCAGGCCGAGAGAGGATGAGGAAGGCCAGGAGGGGCAGGACTCCATGTCCAGGGCCAAGGCCAACTGGCT
GCGTGCCTTCAACAAGGTGCGGATGCAGCTGCAGGAGGCCCGGGGAGAAGGAGAGATGTCTAAATCCCTA
TGGTTCAAAGGCGGCCCAGGGGGCGGTCTCATCATCATCGACAGCATGCCAGACATCCGCAAGAGGAAAC
CTATCCCACTCGTGAGCGACTTGGCCATGTCCCTGGTCCAGTCCAGGAAAGCGGGCATCACCTCGGCCTT
GGCCTCCAGCACGTTGAACAACGAGGAGCTGAAAAACCACGTTTACAAGAAGACCCTGCAAGCCTTAATC
TACCCCATCTCGTGCACGACGCCACACAACTTCGAAGTGTGGACGGCCACCACGCCCACCTACTGCTACG
AGTGCGAGGGGCTGCTGTGGGGCATCGCGAGGCAGGGCATGCGCTGCACCGAGTGCGGTGTCAAGTGCCA
CGAGAAGTGCCAGGACCTGCTCAACGCCGACTGCCTGCAGCGGGCTGCGGAGAAGAGCTCCAAGCACGGG
GCGGAGGACCGGACACAGAACATCATCATGGTGCTCAAGGACCGCATGAAGATCCGGGAGCGCAACAAGC
CCGAGATCTTCGAGCTCATCCAGGAGATCTTCGCGGTGACCAAGACGGCGCACACGCAGCAGATGAAGGC
GGTCAAGCAGAGCGTGCTGGACGGCACGTCCAAGTGGTCCGCCAAGATCAGCATCACCGTGGTCTGCGCC
CAGGGCTTGCAGGCAAAGGACAAGACAGGATCCAGTGACCCCTATGTCACCGTCCAGGTCGGGAAGACCA
AGAAACGGACAAAAACCATCTATGGGAACCTCAACCCGGTGTGGGAGGAGAATTTCCACTTTGAATGTCA
CAATTCCTCCGACCGCATCAAGGTGCGCGTCTGGGACGAGGATGACGACATCAAATCCCGCGTGAAACAG
AGGTTCAAGAGGGAATCTGACGATTTCCTGGGGCAGACGATCATTGAGGTGCGGACGCTCAGCGGCGAGA
TGGACGTGTGGTACAACCTGGACAAGCGAACTGACAAATCTGCCGTGTCGGGTGCCATCCGGCTCCACAT
CAGTGTGGAGATCAAAGGCGAGGAGAAGGTGGCCCCGTACCATGTCCAGTACACCTGTCTGCATGAGAAC
CTGTTCCACTTCGTGACCGACGTGCAGAACAATGGGGTCGTGAAGATCCCAGATGCCAAGGGTGACGATG
CCTGGAAGGTTTACTACGATGAGACAGCCCAGGAGATTGTGGACGAGTTTGCCATGCGCTACGGCGTCGA
GTCCATCTACCAAGCCATGACCCACTTTGCCTGCCTCTCCTCCAAGTATATGTGCCCAGGGGTGCCTGCC
GTCATGAGCACCCTGCTCGCCAACATCAATGCCTACTACGCACACACCACCGCCTCCACCAACGTGTCTG
CCTCCGACCGCTTCGCCGCCTCCAACTTTGGGAAAGAGCGCTTCGTGAAACTCCTGGACCAGCTGCATAA
CTCCCTGCGGATTGACCTCTCCATGTACCGGAATAACTTCCCAGCCAGCAGCCCGGAGAGACTCCAGGAC
CTCAAATCCACTGTGGACCTTCTCACCAGCATCACCTTCTTTCGGATGAAGGTACAAGAACTCCAGAGCC
CGCCCCGAGCCAGCCAGGTGGTAAAGGACTGTGTGAAAGCCTGCCTTAATTCTACCTACGAGTACATCTT
CAATAACTGCCATGAACTGTACAGCCGGGAGTACCAGACAGACCCGGCCAAGAAGGGGGAAGTTCTCCCA
GAGGAACAGGGGCCCAGCATCAAGAACCTCGACTTCTGGTCCAAGCTGATTACCCTCATAGTGTCCATCA
TTGAGGAAGACAAGAATTCCTACACTCCCTGCCTCAACCAGTTTCCCCAGGAGCTGAATGTGGGTAAAAT
CAGCGCTGAAGTGATGTGGAATCTGTTTGCCCAAGACATGAAGTACGCCATGGAGGAGCACGACAAGCAT
CGTCTATGCAAGAGTGCCGACTACATGAACCTCCACTTCAAGGTGAAATGGCTCTACAATGAGTATGTGA
CGGAACTTCCCGCCTTCAAGGACCGCGTGCCTGAGTACCCTGCATGGTTTGAACCCTTCGTCATCCAGTG
GCTGGATGAGAATGAGGAGGTGTCCCGGGATTTCCTGCACGGTGCCCTGGAGCGAGACAAGAAGGATGGG
TTCCAGCAGACCTCAGAGCATGCCCTATTCTCCTGCTCCGTGGTGGATGTTTTCTCCCAACTCAACCAGA
GCTTTGAAATCATCAAGAAACTCGAGTGTCCCGACCCTCAGATCGTGGGGCACTACATGAGGCGCTTTGC
CAAGACCATCAGTAATGTGCTCCTCCAGTATGCAGACATCATCTCCAAGGACTTTGCCTCCTACTGCTCC
AAGGAGAAGGAGAAAGTGCCCTGCATTCTCATGAATAACACTCAACAGCTACGAGTTCAGCTGGAGAAGA
TGTTCGAAGCCATGGGAGGAAAGGAGCTGGATGCTGAAGCCAGTGACATCCTGAAGGAGCTTCAGGTGAA
ACTCAATAACGTCTTGGATGAGCTCAGCCGGGTGTTTGCTACCAGCTTCCAGCCGCACATTGAAGAGTGT
GTCAAACAGATGGGTGACATCCTTAGCCAGGTTAAGGGCACAGGCAATGTGCCAGCCAGTGCCTGCAGCA
GCGTGGCCCAGGACGCGGACAATGTGTTGCAGCCCATCATGGACCTGCTGGACAGCAACCTGACCCTCTT
TGCCAAAATCTGTGAGAAGACTGTGCTGAAGCGAGTGCTGAAGGAGCTGTGGAAGCTGGTTATGAACACC
ATGGAGAAAACCATCGTCCTGCCGCCCCTCACTGACCAGACGATGATCGGGAACCTCTTGAGAAAACATG
GCAAGGGATTAGAAAAGGGCAGGGTGAAATTGCCAAGCCACTCAGACGGAACCCAGATGATCTTCAATGC
AGCCAAGGAGCTGGGTCAGCTGTCCAAACTCAAGGATCACATGGTACGAGAAGAAGCCAAGAGCTTGACC
CCAAAGCAGTGCGCGGTTGTTGAGTTGGCCCTGGACACCATCAAGCAATATTTCCACGCGGGTGGCGTGG
GCCTCAAGAAGACCTTCCTGGAGAAGAGCCCGGACCTGCAATCCTTGCGCTATGCCCTGTCGCTCTACAC
GCAGGCCACCGACCTGCTAATCAAGACCTTTGTACAGACGCAATCGGCCCAGGGCTTGGGTGTAGAAGAC
CCTGTGGGTGAAGTCTCTGTCCATGTTGAGCTGTTCACTCATCCAGGAACTGGGGAACACAAGGTCACAG
TGAAAGTGGTGGCTGCCAATGACCTCAAGTGGCAGACTTCTGGCATCTTCCGGCCGTTCATCGAGGTCAA
CATCATTGGGCCCCAGCTCAGCGACAAGAAACGCAAGTTTGCGACCAAATCCAAGAACAATAGCTGGGCT
CCCAAGTACAATGAGAGCTTCCAGTTCACGCTGAGCGCCGACGCGGGTCCCGAGTGCTATGAGCTGCAGG
TGTGCGTCAAGGACTACTGCTTCGCGCGCGAGGACCGCACGGTGGGGCTGGCCGTGCTGCAGCTGCGTGA
GCTGGCCCAGCGCGGGAGCGCCGCCTGCTGGCTGCCGCTCGGCCGCCGCATCCACATGGACGACACGGGC
CTCACGGTGCTGCGAATCCTCTCGCAGCGCAGCAACGACGAGGTGGCCAAGGAGTTCGTGAAGCTCAAGT
CGGACACGCGCTCCGCCGAGGAGGGCGGTGCCGCGCCTGCGCCTTAGCGCGGGCGGTCGGCCGAGCGGCA
CTGCGCCTGCGCGGAGGGCGCTGGGCGGGGAGGGACGGGGCTTGCGCCTTGGTGGGACCTCCCCAGGGGC
GGGGCTCGGGGGGCTCCACGCCAAGGGTGGGCTGCGCCTACGCCCTTGACTCAGCTTTCCCTTTTGGGGA
ATTAGGAATGGAGGATGCCCCGCCCTCTCGGGAGGCCACGCCCAAGGGCGCGACGAAGGAAGGAGCCACA
TCCCCAACTTGAGGCCACGCCCCCAGCACCTAGGGGGCATTTTGAGCTGGGATGGGGGAAACCTCGTCCC
TATGGAGGAGGCCACATCCCGGGGCTCTGGTACCGGGAGGCACCACCTCATGTCCCCTGGAAAAGCCATA
AGATGGGACCCAGACCCCTGGGACCCCAGACCAATTGCCAAGTATGGAAATCTCAGCTCCCTCGAGGGGG
GGCCCTGGGCAAGGGGTAGGGCTCTCTGGAGCGCCCCTCTAGGTGGCCTGGGGACTGGAGGGACCAGGAT
GCTGGTTGGAGGGCCCCGGAATACCGGAGTCCCTTTAGATATTTGTGCAAAAAATAAATGGGGGGAGGGG
GGAGGATGGGATTTCAAAAGCACATGCGCCCTTGGGCGCCCAAACCCTGGGGGCCGAGGGGACGGCTCTG
GTTCCCCACGCTGCCCCTACTTCCCTTTGGGAGTTTGCCTCTCCCTCTCCCCCAACAAACCCAGTCCTCA
TATCATAGAGTTCAACACACCCATTTGACAGATGGCAAAACTGAGGCTTAAAGAGCTGCTTGAGACTTGG
CCAAGGTTCCAGGTGCCATACCCTCTGTGCCCCTCCCTTAGGCCTGTGTGCCCCATGGAAGGGTGGGCTG
AGATCGGGATGACCTGACACAGCTCCCTATTGCTGCTAATTCCCCCTCGGCCTCCTCCAAGGGGTGGGAA
TTCCAGGCCAAGACCCCTACTTCGCCTTTCCTTCTCCGGCTGCCAAGCAGGACCTTTGCCCTCAGCCCTT
TCTCCTGGGATCTCCATGGGGGATGCCATGAGGGCCTCCCACCACAAAAGAGAATTTGGGATCCCCTGGT
CCCAGGTTTCTCCATCCCTTCTTCCTTTTCCAGAATTTTCCAAATAGGAAAGAACAGAAGGAGACCAGAA
ACTCTAGGGGGGAGAAAGAGAATGAGAGAAAGAGAATGAGAGAGAGAGAAACACAAACACAGTGACACAG
TGAGAGCTTAGTCTCCAAGAGCCTATTCATTGATTCAAACACCCAAGCCACAGGATACCTCAGATGGCCC
TCTTGCCAGCTGGAAGCTCTTTCTCCAATGAGCAAAGTTACAGTGACCTGGCTGGAGTTACCTGGTGCAC
ATAGGACCTTAGGGGAAAGTTCAGCGTGGACTACACTTGCTCTGGGATCTGCTTTTCCACATGTGTGTAT
GGCACGCCTTTTTCTGCTGGATTGGGAAGGACAAGATTTTGCTGTGCTAGGGAGAAATGAAAACGGGGTG
AGCTGAGTAGCTGGGTTTCTGGAGGATAGAACATCAGATGGGGAGGCTTTCCGAGGTGAAGAATGAGAGG
GAACCACTTACTAGAGAGAAAAGAGCTCCAGGCCTGGGGAACAGCACGTGCGAAGGCCAGGAGAGAAGAA
CTGTTGAAACAACGAGAAGGGTGGCACGGCTGGAGCTGAGCCAGCAAGGGGGATCGTGAGGAGCCTTGGG
GTTGGGGAGATCTGCAGAAGCATCAGACCAGGCAGGGCCTCGTACGCAGTCCTGAGGAGTTTTACTTTTA
TTCTAAGACAGTTGGGGAGCTCCAGGAGCTGTTTTAAGTTGGGGAGAGACTGGATTCCAGCCTGCAAAAG
CTGTTTTGTGAAGACTAAAACCAGTGAGGAGAGGTGGAGGTGCTTTGGGGACACTGAAATGGATTCTTGG
AAAGATTCTGAAGGCTGTGTTGAAAAGACACCTATAGCTGTGGGGACATGACTATAATCCCAGCATTTGG
GGAGACCGAGGCTGGCAGATCACTTAAGGTCAGGAGTTTGAGACCAGCCTGGCCAACATGGCGAAACCCC
ATCTCTGCTAAAAATACAAAAATTAGCTGGGTGCAGTGGTGCATGCCTGTAGTCCCAGCTACTCAGGAGA
CTGAGGCGGGAGAATTGTTTGAACCCTGGAGGCAGAGGTTGTAGTGAGTCGTGATCACACAACTGCACTC
CAGCCTGGGCAACAGAACAATACTCCATTCCCTCCCCTCTACCCCACCAAAAAAAAAAAAAAATCCTGCC
CTTAGATGAGCTCTAGGGCTGCTGAGTACAGTTGTCCCAGTTGCACAGTGCCCAAGGGTTTGGCATTGCT
AAGAAGGCCACGTGCAAATCCTAGATATTGAGTGTTGTATGTTTGTGACGTTGGTTTCCCGACATGTGAA
TGGCCCAAGTGTCTGGAAGAAGTGGCGCCACTTTCTAATTTGCTTGGAGATGTTGCATGTCCCTTAAATT
CAGACAGGTGCAGGTAACTGGAGGTTCTGAACCAAAGGTTAAAATGCAAATTCTCATACAGGGTTGGGAA
GTTGTAGCCAGGGATAAGCTTATGTGACTGTTATATGGACTGAGGAGCAGATGTGAATTTCGAACCATGA
CATGGCTGAGGGTAGGGGTCGGGTGGATGGATGATTCAGGGTTGTAACCCATAGAGCCCAAAGGGGAAGT
GATCTGTGACCTGGGGTGAGGGTGATCTGGAAGATTTTTGGATGGCTGGAAAGAAATGGGGAAGTCGAGC
TGCCTGAGAGAGCCAAGTTATTTCCCAAAAGATTCCTTAGGAGTCTTTCTGTTCAAGACCTCCGTGTGTG
TGTGTGTGTGTTTAGGGTTCCCCAGCAATGGCCCAGGCATGTGAAGGAAACAAGCTTCTTCAGGGAATAT
TTGTTGAATGAGTTTTCCTGACTCCCAGGCTAGAACTGTTTTTGCAATTTCCACCCTCTTTTCTTTCCCC
CAGAGAACTCCTATTCGTCCTTCAAAACCCATCACGGAAACCCCTCTTGGAGAAAACCCTCCTTCCTTCC
CCTCAGGACTTTCCCAGCCACCGTCTCTCCTCCAGTCCAGCCTGATGCCATGGGACTGGGGGTTTCTCTG
TCCAGCTCTGTTTCTCCCAGACTGGGGTCTGAGGACTCTCAGGACCCCCAACTTTACCTAGCACAGGCTG
GGCACAAGTGGGTGACAGGGAGTCTACGCCTAGTGGAATTATGTATTGGGGCAGGGTCAGTGTGAGAATA
CACATCCGCATGCATGTCTGTCCATGTCTGTCCGTACCAACCTTCCCCTTCCACACGGACCTGGGCACAT
AGGAGGTGTCTGAGCCTGACACATGGGACAGAGAGTGGACATGGCTGAGACACGGACAGAGAAAAGACAA
GGAGTCCAGGGGGCTGAAAGCCTTTTGAAATCAGGAAGTTCCTGTATTGGCAGAACAAAGCCCAGAGAGG
AGCAGGGCTTTCCTCAACGCCACCCAGCAAGTGGACACAGAGCCCGGCCTTGGATGACACCTCCAGGGTT
CTGAACCCTGGACCTCGCTTTATGCAAGGAGCTGGCCCCACATTTCCATGAATCGGGGAAACAGCACAAG
AAGGTTGGCCTGTGGCAGGGCAAGGGTTAAAGGGGTGACATTGAGGGATGCCTCAGAGTCAAAGTCCCCT
GACCAAGAGGAATAGAGTAGAAAACACAGAGACAGAGGGTGAGATCACGCCCCGATGAGGACGGAGAGAG
ACAGAGATGGAGAGAGACATAGAGGTGGAAATATACAGAGAAAGATAAATGCAGAGACCAAGGCAGGGAG
TGTCGGGGGAAGTAAAGAGGGTGTCCTGAAGAAAGAAGGATCTGTTCACTCTTACCAGTCTGTCCTCGAA
TGATTTGCATAAAATGAGGAGGTGCCTGTCCACACCCCCAATTCCTCTCTCAGGCCCCAGAGCCTGAGAC
CTCACCATGCCCCCATCAGAGATGCAAAAAACTAAACACCCAACTAGAAATCCTTGGGACCTCTCTCGGC
TGGGATCTCAGAGCCTTTCTGTCCCCTACCCCTACCCCATGTGCTGTCGATTTTGCAGATGGGGACAACC
TGGGGCCTCCCGGAACTCTGCCACCCTGGGGAAGTTGGGGGAGGGCCTTAGTCCCGGATCACAACCCCGT
CTGCTCCCCAGAATCCTTTCCTAAGAATCGTTGAGGACCAAAGTTGTCTTTGCTGACACGTGTTGCTTTT
CTCTTTGCCTTTTATTGTTTCAGAGAAAAATCAAGTTGACTGTGTCAAGTAACACCCCACCCCTTACCCC
CGTCCAGCCATAGTGGCTCTCTGGAGACACAGGTCACAGGCGGAGGGTCCCCTGATCATCCCCAACCACA
CAGCCAGGGGGACTTGACCCCTGTCCACCCCTGTCTCGTGCTCCCTCAGACCCCCACAAACCGGCCAAGC
AGTCCGGGGAGGCTTCCCCTCCACACAACTCTTAGCATGTGATTGCAGATGTGAAATCAAAACGTTGTTT
GTTTTTTGTTTTGTTTTGATTCTACCCCGTCGGTCCAGTGTCTGCACAGACGCCTTCATTTCTCTGTAAA
TATGTGACTTGGAACAAATGTTTAACACAAACGAGAAGTGGTCATGAATGCATGGTGTTGAGATGTTTTG
CACTATTCTGACTTTTTGGTCTCTGTAAAAATATTTTATTAACAGCAGACATTAAAAAAAGAAAAACCAC
ACACAGCCTTGGA
(SEQ ID NO: 5061 (SOURCE NCBI Reference Sequence XM_011527810.2)).

In various embodiments, an UNC13A mRNA transcript comprises the sequence provided as SEQ ID NO: 5062.

CTGAACCAAGATGGCCGGTGGCGGCCGGGCCCCGGCGTGAGCCAAGCGCGGGCTGCAGCCGGGAGATGCC
CCAGCCCAGCGGCCGCTGAGCCCGACCCGACAGAGCCGGCCCGGCCGCCTCCGGCCCACCTGCGAGCTCG
GAGACATGTCTCTGCTTTGCGTTGGAGTCAAAAAAGCCAAGTTTGATGGTGCCCAAGAGAAATTCAACAC
GTACGTGACCCTGAAAGTGCAGAATGTCAAGAGCACGACCATCGCGGTGCGGGGCAGCCAGCCCAGCTGG
GAGCAGGATTTCATGTTCGAGATTAACCGTCTGGATTTGGGACTGACGGTGGAGGTGTGGAATAAGGGTC
TCATCTGGGACACAATGGTGGGCACTGTGTGGATCCCACTGAGGACCATCCGCCAGTCCAATGAGGAGGG
CCCTGGAGAGTGGCTGACGCTGGACTCCCAGGTCATCATGGCAGACAGTGAGATCTGTGGCACCAAGGAC
CCCACCTTCCACCGCATCCTCCTGGACACGCGCTTTGAGCTACCCTTAGACATTCCTGAAGAGGAGGCTC
GCTACTGGGCCAAGAAGCTGGAGCAGCTCAATGCTATGCGGGACCAGGATGAATATTCGTTCCAAGATGA
GCAAGACAAGCCTCTGCCTGTCCCCAGCAACCAGTGCTGCAACTGGAATTATTTTGGCTGGGGTGAGCAG
CACAACGATGACCCCGACAGTGCAGTGGATGATCGTGACAGTGACTACCGCAGTGAAACGAGCAACAGCA
TCCCGCCGCCCTATTATACTACGTCACAACCCAACGCCTCAGTCCACCAATATTCTGTTCGCCCACCACC
CCTGGGCTCCCGGGAGTCCTACAGTGACTCCATGCACAGTTACGAGGAGTTCTCTGAGCCACAAGCCCTC
AGCCCCACGGGTAGCAGCCGCTATGCCTCTTCCGGGGAGCTGAGCCAGGGAAGCTCTCAGCTGAGCGAGG
ACTTCGACCCTGACGAGCACAGCCTGCAGGGCTCCGACATGGAGGATGAGCGGGACCGGGACTCCTACCA
CTCCTGCCACAGCTCGGTCAGCTACCACAAAGACTCGCCTCGCTGGGACCAGGATGAGGAAGAGCTGGAG
GAGGACCTGGAGGACTTCCTGGAGGAGGAGGAGCTGCCTGAAGATGAGGAGGAGCTGGAGGAGGAGGAGG
AGGAGGTGCCTGACGATTTGGGCAGCTATGCCCAGCGTGAAGACGTAGCTGTGGCTGAGCCCAAAGACTT
CAAACGCATCAGCCTCCCGCCAGCTGCCCCAGGGAAGGAGGACAAGGCCCCAGTGGCACCCACCGAGGCC
CCCGACATGGCCAAGGTGGCCCCCAAGCCAGCCACGCCCGACAAGGTGCCTGCAGCTGAGCAGATCCCTG
AGGCTGAGCCACCCAAGGACGAGGAGAGTTTCAGGCCGAGAGAGGATGAGGAAGGCCAGGAGGGGCAGGA
CTCCATGTCCAGGGCCAAGGCCAACTGGCTGCGTGCCTTCAACAAGGTGCGGATGCAGCTGCAGGAGGCC
CGGGGAGAAGGAGAGATGTCTAAATCCCTATGGTTCAAAGGCGGCCCAGGGGGCGGTCTCATCATCATCG
ACAGCATGCCAGACATCCGCAAGAGGAAACCTATCCCACTCGTGAGCGACTTGGCCATGTCCCTGGTCCA
GTCCAGGAAAGCGGGCATCACCTCGGCCTTGGCCTCCAGCACGTTGAACAACGAGGAGCTGAAAAACCAC
GTTTACAAGAAGACCCTGCAAGCCTTAATCTACCCCATCTCGTGCACGACGCCACACAACTTCGAAGTGT
GGACGGCCACCACGCCCACCTACTGCTACGAGTGCGAGGGGCTGCTGTGGGGCATCGCGAGGCAGGGCAT
GCGCTGCACCGAGTGCGGTGTCAAGTGCCACGAGAAGTGCCAGGACCTGCTCAACGCCGACTGCCTGCAG
CGGGCTGCGGAGAAGAGCTCCAAGCACGGGGCGGAGGACCGGACACAGAACATCATCATGGTGCTCAAGG
ACCGCATGAAGATCCGGGAGCGCAACAAGCCCGAGATCTTCGAGCTCATCCAGGAGATCTTCGCGGTGAC
CAAGACGGCGCACACGCAGCAGATGAAGGCGGTCAAGCAGAGCGTGCTGGACGGCACGTCCAAGTGGTCC
GCCAAGATCAGCATCACCGTGGTCTGCGCCCAGGGCTTGCAGGCAAAGGACAAGACAGGATCCAGTGACC
CCTATGTCACCGTCCAGGTCGGGAAGACCAAGAAACGGACAAAAACCATCTATGGGAACCTCAACCCGGT
GTGGGAGGAGAATTTCCACTTTGAATGTCACAATTCCTCCGACCGCATCAAGGTGCGCGTCTGGGACGAG
GATGACGACATCAAATCCCGCGTGAAACAGAGGTTCAAGAGGGAATCTGACGATTTCCTGGGGCAGACGA
TCATTGAGGTGCGGACGCTCAGCGGCGAGATGGACGTGTGGTACAACCTGGACAAGCGAACTGACAAATC
TGCCGTGTCGGGTGCCATCCGGCTCCACATCAGTGTGGAGATCAAAGGCGAGGAGAAGGTGGCCCCGTAC
CATGTCCAGTACACCTGTCTGCATGAGAACCTGTTCCACTTCGTGACCGACGTGCAGAACAATGGGGTCG
TGAAGATCCCAGATGCCAAGGGTGACGATGCCTGGAAGGTTTACTACGATGAGACAGCCCAGGAGATTGT
GGACGAGTTTGCCATGCGCTACGGCGTCGAGTCCATCTACCAAGCCATGACCCACTTTGCCTGCCTCTCC
TCCAAGTATATGTGCCCAGGGGTGCCTGCCGTCATGAGCACCCTGCTCGCCAACATCAATGCCTACTACG
CACACACCACCGCCTCCACCAACGTGTCTGCCTCCGACCGCTTCGCCGCCTCCAACTTTGGGAAAGAGCG
CTTCGTGAAACTCCTGGACCAGCTGCATAACTCCCTGCGGATTGACCTCTCCATGTACCGGAATAACTTC
CCAGCCAGCAGCCCGGAGAGACTCCAGGACCTCAAATCCACTGTGGACCTTCTCACCAGCATCACCTTCT
TTCGGATGAAGGTACAAGAACTCCAGAGCCCGCCCCGAGCCAGCCAGGTGGTAAAGGACTGTGTGAAAGC
CTGCCTTAATTCTACCTACGAGTACATCTTCAATAACTGCCATGAACTGTACAGCCGGGAGTACCAGACA
GACCCGGCCAAGAAGGGGGAAGTTCTCCCAGAGGAACAGGGGCCCAGCATCAAGAACCTCGACTTCTGGT
CCAAGCTGATTACCCTCATAGTGTCCATCATTGAGGAAGACAAGAATTCCTACACTCCCTGCCTCAACCA
GTTTCCCCAGGAGCTGAATGTGGGTAAAATCAGCGCTGAAGTGATGTGGAATCTGTTTGCCCAAGACATG
AAGTACGCCATGGAGGAGCACGACAAGCATCGTCTATGCAAGAGTGCCGACTACATGAACCTCCACTTCA
AGGTGAAATGGCTCTACAATGAGTATGTGACGGAACTTCCCGCCTTCAAGGACCGCGTGCCTGAGTACCC
TGCATGGTTTGAACCCTTCGTCATCCAGTGGCTGGATGAGAATGAGGAGGTGTCCCGGGATTTCCTGCAC
GGTGCCCTGGAGCGAGACAAGAAGGATGGGTTCCAGCAGACCTCAGAGCATGCCCTATTCTCCTGCTCCG
TGGTGGATGTTTTCTCCCAACTCAACCAGAGCTTTGAAATCATCAAGAAACTCGAGTGTCCCGACCCTCA
GATCGTGGGGCACTACATGAGGCGCTTTGCCAAGACCATCAGTAATGTGCTCCTCCAGTATGCAGACATC
ATCTCCAAGGACTTTGCCTCCTACTGCTCCAAGGAGAAGGAGAAACCCTGCATTCTCATGAATAACACTC
AACAGCTACGAGTTCAGCTGGAGAAGATGTTCGAAGCCATGGGAGGAAAGGAGCTGGATGCTGAAGCCAG
TGACATCCTGAAGGAGCTTCAGGTGAAACTCAATAACGTCTTGGATGAGCTCAGCCGGGTGTTTGCTACC
AGCTTCCAGCCGCACATTGAAGAGTGTGTCAAACAGATGGGTGACATCCTTAGCCAGGTTAAGGGCACAG
GCAATGTGCCAGCCAGTGCCTGCAGCAGCGTGGCCCAGGACGCGGACAATGTGTTGCAGCCCATCATGGA
CCTGCTGGACAGCAACCTGACCCTCTTTGCCAAAATCTGTGAGAAGACTGTGCTGAAGCGAGTGCTGAAG
GAGCTGTGGAAGCTGGTTATGAACACCATGGAGAAAACCATCGTCCTGCCGCCCCTCACTGACCAGACGA
TGATCGGGAACCTCTTGAGAAAACATGGCAAGGGATTAGAAAAGGGCAGGGTGAAATTGCCAAGCCACTC
AGACGGAACCCAGATGATCTTCAATGCAGCCAAGGAGCTGGGTCAGCTGTCCAAACTCAAGGATCACATG
GTACGAGAAGAAGCCAAGAGCTTGACCCCAAAGCAGTGCGCGGTTGTTGAGTTGGCCCTGGACACCATCA
AGCAATATTTCCACGCGGGTGGCGTGGGCCTCAAGAAGACCTTCCTGGAGAAGAGCCCGGACCTGCAATC
CTTGCGCTATGCCCTGTCGCTCTACACGCAGGCCACCGACCTGCTAATCAAGACCTTTGTACAGACGCAA
TCGGCCCAGGGCTTGGGTGTAGAAGACCCTGTGGGTGAAGTCTCTGTCCATGTTGAGCTGTTCACTCATC
CAGGAACTGGGGAACACAAGGTCACAGTGAAAGTGGTGGCTGCCAATGACCTCAAGTGGCAGACTTCTGG
CATCTTCCGGCCGTTCATCGAGGTCAACATCATTGGGCCCCAGCTCAGCGACAAGAAACGCAAGTTTGCG
ACCAAATCCAAGAACAATAGCTGGGCTCCCAAGTACAATGAGAGCTTCCAGTTCACGCTGAGCGCCGACG
CGGGTCCCGAGTGCTATGAGCTGCAGGTGTGCGTCAAGGACTACTGCTTCGCGCGCGAGGACCGCACGGT
GGGGCTGGCCGTGCTGCAGCTGCGTGAGCTGGCCCAGCGCGGGAGCGCCGCCTGCTGGCTGCCGCTCGGC
CGCCGCATCCACATGGACGACACGGGCCTCACGGTGCTGCGAATCCTCTCGCAGCGCAGCAACGACGAGG
TGGCCAAGGAGTTCGTGAAGCTCAAGTCGGACACGCGCTCCGCCGAGGAGGGCGGTGCCGCGCCTGCGCC
TTAGCGCGGGCGGTCGGCCGAGCGGCACTGCGCCTGCGCGGAGGGCGCTGGGGGGGGAGGGACGGGGCTT
GCGCCTTGGTGGGACCTCCCCAGGGGCGGGGCTCGGGGGGCTCCACGCCAAGGGTGGGCTGCGCCTACGC
CCTTGACTCAGCTTTCCCTTTTGGGGAATTAGGAATGGAGGATGCCCCGCCCTCTCGGGAGGCCACGCCC
AAGGGCGCGACGAAGGAAGGAGCCACATCCCCAACTTGAGGCCACGCCCCCAGCACCTAGGGGGCATTTT
GAGCTGGGATGGGGGAAACCTCGTCCCTATGGAGGAGGCCACATCCCGGGGCTCTGGTACCGGGAGGCAC
CACCTCATGTCCCCTGGAAAAGCCATAAGATGGGACCCAGACCCCTGGGACCCCAGACCAATTGCCAAGT
ATGGAAATCTCAGCTCCCTCGAGGGGGGGCCCTGGGCAAGGGGTAGGGCTCTCTGGAGCGCCCCTCTAGG
TGGCCTGGGGACTGGAGGGACCAGGATGCTGGTTGGAGGGCCCCGGAATACCGGAGTCCCTTTAGATATT
TGTGCAAAAAATAAATGGGGGGAGGGGGGAGGATGGGATTTCAAAAGCACATGCGCCCTTGGGCGCCCAA
ACCCTGGGGGCCGAGGGGACGGCTCTGGTTCCCCACGCTGCCCCTACTTCCCTTTGGGAGTTTGCCTCTC
CCTCTCCCCCAACAAACCCAGTCCTCATATCATAGAGTTCAACACACCCATTTGACAGATGGCAAAACTG
AGGCTTAAAGAGCTGCTTGAGACTTGGCCAAGGTTCCAGGTGCCATACCCTCTGTGCCCCTCCCTTAGGC
CTGTGTGCCCCATGGAAGGGTGGGCTGAGATCGGGATGACCTGACACAGCTCCCTATTGCTGCTAATTCC
CCCTCGGCCTCCTCCAAGGGGTGGGAATTCCAGGCCAAGACCCCTACTTCGCCTTTCCTTCTCCGGCTGC
CAAGCAGGACCTTTGCCCTCAGCCCTTTCTCCTGGGATCTCCATGGGGGATGCCATGAGGGCCTCCCACC
ACAAAAGAGAATTTGGGATCCCCTGGTCCCAGGTTTCTCCATCCCTTCTTCCTTTTCCAGAATTTTCCAA
ATAGGAAAGAACAGAAGGAGACCAGAAACTCTAGGGGGGAGAAAGAGAATGAGAGAAAGAGAATGAGAGA
GAGAGAAACACAAACACAGTGACACAGTGAGAGCTTAGTCTCCAAGAGCCTATTCATTGATTCAAACACC
CAAGCCACAGGATACCTCAGATGGCCCTCTTGCCAGCTGGAAGCTCTTTCTCCAATGAGCAAAGTTACAG
TGACCTGGCTGGAGTTACCTGGTGCACATAGGACCTTAGGGGAAAGTTCAGCGTGGACTACACTTGCTCT
GGGATCTGCTTTTCCACATGTGTGTATGGCACGCCTTTTTCTGCTGGATTGGGAAGGACAAGATTTTGCT
GTGCTAGGGAGAAATGAAAACGGGGTGAGCTGAGTAGCTGGGTTTCTGGAGGATAGAACATCAGATGGGG
AGGCTTTCCGAGGTGAAGAATGAGAGGGAACCACTTACTAGAGAGAAAAGAGCTCCAGGCCTGGGGAACA
GCACGTGCGAAGGCCAGGAGAGAAGAACTGTTGAAACAACGAGAAGGGTGGCACGGCTGGAGCTGAGCCA
GCAAGGGGGATCGTGAGGAGCCTTGGGGTTGGGGAGATCTGCAGAAGCATCAGACCAGGCAGGGCCTCGT
ACGCAGTCCTGAGGAGTTTTACTTTTATTCTAAGACAGTTGGGGAGCTCCAGGAGCTGTTTTAAGTTGGG
GAGAGACTGGATTCCAGCCTGCAAAAGCTGTTTTGTGAAGACTAAAACCAGTGAGGAGAGGTGGAGGTGC
TTTGGGGACACTGAAATGGATTCTTGGAAAGATTCTGAAGGCTGTGTTGAAAAGACACCTATAGCTGTGG
GGACATGACTATAATCCCAGCATTTGGGGAGACCGAGGCTGGCAGATCACTTAAGGTCAGGAGTTTGAGA
CCAGCCTGGCCAACATGGCGAAACCCCATCTCTGCTAAAAATACAAAAATTAGCTGGGTGCAGTGGTGCA
TGCCTGTAGTCCCAGCTACTCAGGAGACTGAGGCGGGAGAATTGTTTGAACCCTGGAGGCAGAGGTTGTA
GTGAGTCGTGATCACACAACTGCACTCCAGCCTGGGCAACAGAACAATACTCCATTCCCTCCCCTCTACC
CCACCAAAAAAAAAAAAAAATCCTGCCCTTAGATGAGCTCTAGGGCTGCTGAGTACAGTTGTCCCAGTTG
CACAGTGCCCAAGGGTTTGGCATTGCTAAGAAGGCCACGTGCAAATCCTAGATATTGAGTGTTGTATGTT
TGTGACGTTGGTTTCCCGACATGTGAATGGCCCAAGTGTCTGGAAGAAGTGGCGCCACTTTCTAATTTGC
TTGGAGATGTTGCATGTCCCTTAAATTCAGACAGGTGCAGGTAACTGGAGGTTCTGAACCAAAGGTTAAA
ATGCAAATTCTCATACAGGGTTGGGAAGTTGTAGCCAGGGATAAGCTTATGTGACTGTTATATGGACTGA
GGAGCAGATGTGAATTTCGAACCATGACATGGCTGAGGGTAGGGGTCGGGTGGATGGATGATTCAGGGTT
GTAACCCATAGAGCCCAAAGGGGAAGTGATCTGTGACCTGGGGTGAGGGTGATCTGGAAGATTTTTGGAT
GGCTGGAAAGAAATGGGGAAGTCGAGCTGCCTGAGAGAGCCAAGTTATTTCCCAAAAGATTCCTTAGGAG
TCTTTCTGTTCAAGACCTCCGTGTGTGTGTGTGTGTGTTTAGGGTTCCCCAGCAATGGCCCAGGCATGTG
AAGGAAACAAGCTTCTTCAGGGAATATTTGTTGAATGAGTTTTCCTGACTCCCAGGCTAGAACTGTTTTT
GCAATTTCCACCCTCTTTTCTTTCCCCCAGAGAACTCCTATTCGTCCTTCAAAACCCATCACGGAAACCC
CTCTTGGAGAAAACCCTCCTTCCTTCCCCTCAGGACTTTCCCAGCCACCGTCTCTCCTCCAGTCCAGCCT
GATGCCATGGGACTGGGGGTTTCTCTGTCCAGCTCTGTTTCTCCCAGACTGGGGTCTGAGGACTCTCAGG
ACCCCCAACTTTACCTAGCACAGGCTGGGCACAAGTGGGTGACAGGGAGTCTACGCCTAGTGGAATTATG
TATTGGGGCAGGGTCAGTGTGAGAATACACATCCGCATGCATGTCTGTCCATGTCTGTCCGTACCAACCT
TCCCCTTCCACACGGACCTGGGCACATAGGAGGTGTCTGAGCCTGACACATGGGACAGAGAGTGGACATG
GCTGAGACACGGACAGAGAAAAGACAAGGAGTCCAGGGGGCTGAAAGCCTTTTGAAATCAGGAAGTTCCT
GTATTGGCAGAACAAAGCCCAGAGAGGAGCAGGGCTTTCCTCAACGCCACCCAGCAAGTGGACACAGAGC
CCGGCCTTGGATGACACCTCCAGGGTTCTGAACCCTGGACCTCGCTTTATGCAAGGAGCTGGCCCCACAT
TTCCATGAATCGGGGAAACAGCACAAGAAGGTTGGCCTGTGGCAGGGCAAGGGTTAAAGGGGTGACATTG
AGGGATGCCTCAGAGTCAAAGTCCCCTGACCAAGAGGAATAGAGTAGAAAACACAGAGACAGAGGGTGAG
ATCACGCCCCGATGAGGACGGAGAGAGACAGAGATGGAGAGAGACATAGAGGTGGAAATATACAGAGAAA
GATAAATGCAGAGACCAAGGCAGGGAGTGTCGGGGGAAGTAAAGAGGGTGTCCTGAAGAAAGAAGGATCT
GTTCACTCTTACCAGTCTGTCCTCGAATGATTTGCATAAAATGAGGAGGTGCCTGTCCACACCCCCAATT
CCTCTCTCAGGCCCCAGAGCCTGAGACCTCACCATGCCCCCATCAGAGATGCAAAAAACTAAACACCCAA
CTAGAAATCCTTGGGACCTCTCTCGGCTGGGATCTCAGAGCCTTTCTGTCCCCTACCCCTACCCCATGTG
CTGTCGATTTTGCAGATGGGGACAACCTGGGGCCTCCCGGAACTCTGCCACCCTGGGGAAGTTGGGGGAG
GGCCTTAGTCCCGGATCACAACCCCGTCTGCTCCCCAGAATCCTTTCCTAAGAATCGTTGAGGACCAAAG
TTGTCTTTGCTGACACGTGTTGCTTTTCTCTTTGCCTTTTATTGTTTCAGAGAAAAATCAAGTTGACTGT
GTCAAGTAACACCCCACCCCTTACCCCCGTCCAGCCATAGTGGCTCTCTGGAGACACAGGTCACAGGCGG
AGGGTCCCCTGATCATCCCCAACCACACAGCCAGGGGGACTTGACCCCTGTCCACCCCTGTCTCGTGCTC
CCTCAGACCCCCACAAACCGGCCAAGCAGTCCGGGGAGGCTTCCCCTCCACACAACTCTTAGCATGTGAT
TGCAGATGTGAAATCAAAACGTTGTTTGTTTTTTGTTTTGTTTTGATTCTACCCCGTCGGTCCAGTGTCT
GCACAGACGCCTTCATTTCTCTGTAAATATGTGACTTGGAACAAATGTTTAACACAAACGAGAAGTGGTC
ATGAATGCATGGTGTTGAGATGTTTTGCACTATTCTGACTTTTTGGTCTCTGTAAAAATATTTTATTAAC
AGCAGACATTAAAAAAAGAAAAACCACACACAGCCTTGGA
(SEQ ID NO: 5062 (SOURCE (NCBI Reference Sequence XM_017026502.1)).

In various embodiments, an UNC13A mRNA transcript comprises the sequence provided as SEQ ID NO: 5063.

CTGAACCAAGATGGCCGGTGGCGGCCGGGCCCCGGCGTGAGCCAAGCGCGGGCTGCAGCCGGGAGATGCC
CCAGCCCAGCGGCCGCTGAGCCCGACCCGACAGAGCCGGCCCGGCCGCCTCCGGCCCACCTGCGAGCTCG
GAGACATGTCTCTGCTTTGCGTTGGAGTCAAAAAAGCCAAGTTTGATGGTGCCCAAGAGAAATTCAACAC
GTACGTGACCCTGAAAGTGCAGAATGTCAAGAGCACGACCATCGCGGTGCGGGGCAGCCAGCCCAGCTGG
GAGCAGGATTTCATGTTCGAGATTAACCGTCTGGATTTGGGACTGACGGTGGAGGTGTGGAATAAGGGTC
TCATCTGGGACACAATGGTGGGCACTGTGTGGATCCCACTGAGGACCATCCGCCAGTCCAATGAGGAGGG
CCCTGGAGAGTGGCTGACGCTGGACTCCCAGGTCATCATGGCAGACAGTGAGATCTGTGGCACCAAGGAC
CCCACCTTCCACCGCATCCTCCTGGACACGCGCTTTGAGCTACCCTTAGACATTCCTGAAGAGGAGGCTC
GCTACTGGGCCAAGAAGCTGGAGCAGCTCAATGCTATGCGGGACCAGGATGAATATTCGTTCCAAGATGA
GCAAGACAAGCCTCTGCCTGTCCCCAGCAACCAGTGCTGCAACTGGAATTATTTTGGCTGGGGTGAGCAG
CACAACGATGACCCCGACAGTGCAGTGGATGATCGTGACAGTGACTACCGCAGTGAAACGAGCAACAGCA
TCCCGCCGCCCTATTATACTACGTCACAACCCAACGCCTCAGTCCACCAATATTCTGTTCGCCCACCACC
CCTGGGCTCCCGGGAGTCCTACAGTGACTCCATGCACAGTTACGAGGAGTTCTCTGAGCCACAAGCCCTC
AGCCCCACGGGTAGCAGCCGCTATGCCTCTTCCGGGGAGCTGAGCCAGGGAAGCTCTCAGCTGAGCGAGG
ACTTCGACCCTGACGAGCACAGCCTGCAGGGCTCCGACATGGAGGATGAGCGGGACCGGGACTCCTACCA
CTCCTGCCACAGCTCGGTCAGCTACCACAAAGACTCGCCTCGCTGGGACCAGGATGAGGAAGAGCTGGAG
GAGGACCTGGAGGACTTCCTGGAGGAGGAGGAGCTGCCTGAAGATGAGGAGGAGCTGGAGGAGGAGGAGG
AGGAGGTGCCTGACGATTTGGGCAGCTATGCCCAGCGTGAAGACGTAGCTGTGGCTGAGCCCAAAGACTT
CAAACGCATCAGCCTCCCGCCAGCTGCCCCAGGGAAGGAGGACAAGGCCCCAGTGGCACCCACCGAGGCC
CCCGACATGGCCAAGGTGGCCCCCAAGCCAGCCACGCCCGACAAGGTGCCTGCAGCTGAGCAGATCCCTG
AGGCTGAGCCACCCAAGGACGAGGAGAGTTTCAGGCCGAGAGAGGATGAGGAAGGCCAGGAGGGGCAGGA
CTCCATGTCCAGGGCCAAGGCCAACTGGCTGCGTGCCTTCAACAAGGTGCGGATGCAGCTGCAGGAGGCC
CGGGGAGAAGGAGAGATGTCTAAATCCCTATGGTTCAAAGGCGGCCCAGGGGGCGGTCTCATCATCATCG
ACAGCATGCCAGACATCCGCAAGAGGAAACCTATCCCACTCGTGAGCGACTTGGCCATGTCCCTGGTCCA
GTCCAGGAAAGCGGGCATCACCTCGGCCTTGGCCTCCAGCACGTTGAACAACGAGGAGCTGAAAAACCAC
GTTTACAAGAAGACCCTGCAAGCCTTAATCTACCCCATCTCGTGCACGACGCCACACAACTTCGAAGTGT
GGACGGCCACCACGCCCACCTACTGCTACGAGTGCGAGGGGCTGCTGTGGGGCATCGCGAGGCAGGGCAT
GCGCTGCACCGAGTGCGGTGTCAAGTGCCACGAGAAGTGCCAGGACCTGCTCAACGCCGACTGCCTGCAG
CGGGCTGCGGAGAAGAGCTCCAAGCACGGGGCGGAGGACCGGACACAGAACATCATCATGGTGCTCAAGG
ACCGCATGAAGATCCGGGAGCGCAACAAGCCCGAGATCTTCGAGCTCATCCAGGAGATCTTCGCGGTGAC
CAAGACGGCGCACACGCAGCAGATGAAGGCGGTCAAGCAGAGCGTGCTGGACGGCACGTCCAAGTGGTCC
GCCAAGATCAGCATCACCGTGGTCTGCGCCCAGGGCTTGCAGGCAAAGGACAAGACAGGATCCAGTGACC
CCTATGTCACCGTCCAGGTCGGGAAGACCAAGAAACGGACAAAAACCATCTATGGGAACCTCAACCCGGT
GTGGGAGGAGAATTTCCACTTTGAATGTCACAATTCCTCCGACCGCATCAAGGTGCGCGTCTGGGACGAG
GATGACGACATCAAATCCCGCGTGAAACAGAGGTTCAAGAGGGAATCTGACGATTTCCTGGGGCAGACGA
TCATTGAGGTGCGGACGCTCAGCGGCGAGATGGACGTGTGGTACAACCTGGACAAGCGAACTGACAAATC
TGCCGTGTCGGGTGCCATCCGGCTCCACATCAGTGTGGAGATCAAAGGCGAGGAGAAGGTGGCCCCGTAC
CATGTCCAGTACACCTGTCTGCATGAGAACCTGTTCCACTTCGTGACCGACGTGCAGAACAATGGGGTCG
TGAAGATCCCAGATGCCAAGGGTGACGATGCCTGGAAGGTTTACTACGATGAGACAGCCCAGGAGATTGT
GGACGAGTTTGCCATGCGCTACGGCGTCGAGTCCATCTACCAAGCCATGACCCACTTTGCCTGCCTCTCC
TCCAAGTATATGTGCCCAGGGGTGCCTGCCGTCATGAGCACCCTGCTCGCCAACATCAATGCCTACTACG
CACACACCACCGCCTCCACCAACGTGTCTGCCTCCGACCGCTTCGCCGCCTCCAACTTTGGGAAAGAGCG
CTTCGTGAAACTCCTGGACCAGCTGCATAACTCCCTGCGGATTGACCTCTCCATGTACCGGAATAACTTC
CCAGCCAGCAGCCCGGAGAGACTCCAGGACCTCAAATCCACTGTGGACCTTCTCACCAGCATCACCTTCT
TTCGGATGAAGGTACAAGAACTCCAGAGCCCGCCCCGAGCCAGCCAGGTGGTAAAGGACTGTGTGAAAGC
CTGCCTTAATTCTACCTACGAGTACATCTTCAATAACTGCCATGAACTGTACAGCCGGGAGTACCAGACA
GACCCGGCCAAGAAGGGGGAAGTTCTCCCAGAGGAACAGGGGCCCAGCATCAAGAACCTCGACTTCTGGT
CCAAGCTGATTACCCTCATAGTGTCCATCATTGAGGAAGACAAGAATTCCTACACTCCCTGCCTCAACCA
GTTTCCCCAGGAGCTGAATGTGGGTAAAATCAGCGCTGAAGTGATGTGGAATCTGTTTGCCCAAGACATG
AAGTACGCCATGGAGGAGCACGACAAGCATCGTCTATGCAAGAGTGCCGACTACATGAACCTCCACTTCA
AGGTGAAATGGCTCTACAATGAGTATGTGACGGAACTTCCCGCCTTCAAGGACCGCGTGCCTGAGTACCC
TGCATGGTTTGAACCCTTCGTCATCCAGTGGCTGGATGAGAATGAGGAGGTGTCCCGGGATTTCCTGCAC
GGTGCCCTGGAGCGAGACAAGAAGGATGGGTTCCAGCAGACCTCAGAGCATGCCCTATTCTCCTGCTCCG
TGGTGGATGTTTTCTCCCAACTCAACCAGAGCTTTGAAATCATCAAGAAACTCGAGTGTCCCGACCCTCA
GATCGTGGGGCACTACATGAGGCGCTTTGCCAAGACCATCAGTAATGTGCTCCTCCAGTATGCAGACATC
ATCTCCAAGGACTTTGCCTCCTACTGCTCCAAGGAGAAGGAGAAAGTGCCCTGCATTCTCATGAATAACA
CTCAACAGCTACGAGTTCAGCTGGAGAAGATGTTCGAAGCCATGGGAGGAAAGGAGCTGGATGCTGAAGC
CAGTGACATCCTGAAGGAGCTTCAGGTGAAACTCAATAACGTCTTGGATGAGCTCAGCCGGGTGTTTGCT
ACCAGCTTCCAGCCGCACATTGAAGAGTGTGTCAAACAGATGGGTGACATCCTTAGCCAGGTTAAGGGCA
CAGGCAATGTGCCAGCCAGTGCCTGCAGCAGCGTGGCCCAGGACGCGGACAATGTGTTGCAGCCCATCAT
GGACCTGCTGGACAGCAACCTGACCCTCTTTGCCAAAATCTGTGAGAAGACTGTGCTGAAGCGAGTGCTG
AAGGAGCTGTGGAAGCTGGTTATGAACACCATGGAGAAAACCATCGTCCTGCCGCCCCTCACTGACCAGA
CGATGATCGGAACCCAGATGATCTTCAATGCAGCCAAGGAGCTGGGTCAGCTGTCCAAACTCAAGGATCA
CATGGTACGAGAAGAAGCCAAGAGCTTGACCCCAAAGCAGTGCGCGGTTGTTGAGTTGGCCCTGGACACC
ATCAAGCAATATTTCCACGCGGGTGGCGTGGGCCTCAAGAAGACCTTCCTGGAGAAGAGCCCGGACCTGC
AATCCTTGCGCTATGCCCTGTCGCTCTACACGCAGGCCACCGACCTGCTAATCAAGACCTTTGTACAGAC
GCAATCGGCCCAGGGCTTGGGTGTAGAAGACCCTGTGGGTGAAGTCTCTGTCCATGTTGAGCTGTTCACT
CATCCAGGAACTGGGGAACACAAGGTCACAGTGAAAGTGGTGGCTGCCAATGACCTCAAGTGGCAGACTT
CTGGCATCTTCCGGCCGTTCATCGAGGTCAACATCATTGGGCCCCAGCTCAGCGACAAGAAACGCAAGTT
TGCGACCAAATCCAAGAACAATAGCTGGGCTCCCAAGTACAATGAGAGCTTCCAGTTCACGCTGAGCGCC
GACGCGGGTCCCGAGTGCTATGAGCTGCAGGTGTGCGTCAAGGACTACTGCTTCGCGCGCGAGGACCGCA
CGGTGGGGCTGGCCGTGCTGCAGCTGCGTGAGCTGGCCCAGCGCGGGAGCGCCGCCTGCTGGCTGCCGCT
CGGCCGCCGCATCCACATGGACGACACGGGCCTCACGGTGCTGCGAATCCTCTCGCAGCGCAGCAACGAC
GAGGTGGCCAAGGAGTTCGTGAAGCTCAAGTCGGACACGCGCTCCGCCGAGGAGGGCGGTGCCGCGCCTG
CGCCTTAGCGCGGGCGGTCGGCCGAGCGGCACTGCGCCTGCGCGGAGGGCGCTGGGCGGGGAGGGACGGG
GCTTGCGCCTTGGTGGGACCTCCCCAGGGGCGGGGCTCGGGGGGCTCCACGCCAAGGGTGGGCTGCGCCT
ACGCCCTTGACTCAGCTTTCCCTTTTGGGGAATTAGGAATGGAGGATGCCCCGCCCTCTCGGGAGGCCAC
GCCCAAGGGCGCGACGAAGGAAGGAGCCACATCCCCAACTTGAGGCCACGCCCCCAGCACCTAGGGGGCA
TTTTGAGCTGGGATGGGGGAAACCTCGTCCCTATGGAGGAGGCCACATCCCGGGGCTCTGGTACCGGGAG
GCACCACCTCATGTCCCCTGGAAAAGCCATAAGATGGGACCCAGACCCCTGGGACCCCAGACCAATTGCC
AAGTATGGAAATCTCAGCTCCCTCGAGGGGGGGCCCTGGGCAAGGGGTAGGGCTCTCTGGAGCGCCCCTC
TAGGTGGCCTGGGGACTGGAGGGACCAGGATGCTGGTTGGAGGGCCCCGGAATACCGGAGTCCCTTTAGA
TATTTGTGCAAAAAATAAATGGGGGGAGGGGGGAGGATGGGATTTCAAAAGCACATGCGCCCTTGGGCGC
CCAAACCCTGGGGGCCGAGGGGACGGCTCTGGTTCCCCACGCTGCCCCTACTTCCCTTTGGGAGTTTGCC
TCTCCCTCTCCCCCAACAAACCCAGTCCTCATATCATAGAGTTCAACACACCCATTTGACAGATGGCAAA
ACTGAGGCTTAAAGAGCTGCTTGAGACTTGGCCAAGGTTCCAGGTGCCATACCCTCTGTGCCCCTCCCTT
AGGCCTGTGTGCCCCATGGAAGGGTGGGCTGAGATCGGGATGACCTGACACAGCTCCCTATTGCTGCTAA
TTCCCCCTCGGCCTCCTCCAAGGGGTGGGAATTCCAGGCCAAGACCCCTACTTCGCCTTTCCTTCTCCGG
CTGCCAAGCAGGACCTTTGCCCTCAGCCCTTTCTCCTGGGATCTCCATGGGGGATGCCATGAGGGCCTCC
CACCACAAAAGAGAATTTGGGATCCCCTGGTCCCAGGTTTCTCCATCCCTTCTTCCTTTTCCAGAATTTT
CCAAATAGGAAAGAACAGAAGGAGACCAGAAACTCTAGGGGGGAGAAAGAGAATGAGAGAAAGAGAATGA
GAGAGAGAGAAACACAAACACAGTGACACAGTGAGAGCTTAGTCTCCAAGAGCCTATTCATTGATTCAAA
CACCCAAGCCACAGGATACCTCAGATGGCCCTCTTGCCAGCTGGAAGCTCTTTCTCCAATGAGCAAAGTT
ACAGTGACCTGGCTGGAGTTACCTGGTGCACATAGGACCTTAGGGGAAAGTTCAGCGTGGACTACACTTG
CTCTGGGATCTGCTTTTCCACATGTGTGTATGGCACGCCTTTTTCTGCTGGATTGGGAAGGACAAGATTT
TGCTGTGCTAGGGAGAAATGAAAACGGGGTGAGCTGAGTAGCTGGGTTTCTGGAGGATAGAACATCAGAT
GGGGAGGCTTTCCGAGGTGAAGAATGAGAGGGAACCACTTACTAGAGAGAAAAGAGCTCCAGGCCTGGGG
AACAGCACGTGCGAAGGCCAGGAGAGAAGAACTGTTGAAACAACGAGAAGGGTGGCACGGCTGGAGCTGA
GCCAGCAAGGGGGATCGTGAGGAGCCTTGGGGTTGGGGAGATCTGCAGAAGCATCAGACCAGGCAGGGCC
TCGTACGCAGTCCTGAGGAGTTTTACTTTTATTCTAAGACAGTTGGGGAGCTCCAGGAGCTGTTTTAAGT
TGGGGAGAGACTGGATTCCAGCCTGCAAAAGCTGTTTTGTGAAGACTAAAACCAGTGAGGAGAGGTGGAG
GTGCTTTGGGGACACTGAAATGGATTCTTGGAAAGATTCTGAAGGCTGTGTTGAAAAGACACCTATAGCT
GTGGGGACATGACTATAATCCCAGCATTTGGGGAGACCGAGGCTGGCAGATCACTTAAGGTCAGGAGTTT
GAGACCAGCCTGGCCAACATGGCGAAACCCCATCTCTGCTAAAAATACAAAAATTAGCTGGGTGCAGTGG
TGCATGCCTGTAGTCCCAGCTACTCAGGAGACTGAGGCGGGAGAATTGTTTGAACCCTGGAGGCAGAGGT
TGTAGTGAGTCGTGATCACACAACTGCACTCCAGCCTGGGCAACAGAACAATACTCCATTCCCTCCCCTC
TACCCCACCAAAAAAAAAAAAAAATCCTGCCCTTAGATGAGCTCTAGGGCTGCTGAGTACAGTTGTCCCA
GTTGCACAGTGCCCAAGGGTTTGGCATTGCTAAGAAGGCCACGTGCAAATCCTAGATATTGAGTGTTGTA
TGTTTGTGACGTTGGTTTCCCGACATGTGAATGGCCCAAGTGTCTGGAAGAAGTGGCGCCACTTTCTAAT
TTGCTTGGAGATGTTGCATGTCCCTTAAATTCAGACAGGTGCAGGTAACTGGAGGTTCTGAACCAAAGGT
TAAAATGCAAATTCTCATACAGGGTTGGGAAGTTGTAGCCAGGGATAAGCTTATGTGACTGTTATATGGA
CTGAGGAGCAGATGTGAATTTCGAACCATGACATGGCTGAGGGTAGGGGTCGGGTGGATGGATGATTCAG
GGTTGTAACCCATAGAGCCCAAAGGGGAAGTGATCTGTGACCTGGGGTGAGGGTGATCTGGAAGATTTTT
GGATGGCTGGAAAGAAATGGGGAAGTCGAGCTGCCTGAGAGAGCCAAGTTATTTCCCAAAAGATTCCTTA
GGAGTCTTTCTGTTCAAGACCTCCGTGTGTGTGTGTGTGTGTTTAGGGTTCCCCAGCAATGGCCCAGGCA
TGTGAAGGAAACAAGCTTCTTCAGGGAATATTTGTTGAATGAGTTTTCCTGACTCCCAGGCTAGAACTGT
TTTTGCAATTTCCACCCTCTTTTCTTTCCCCCAGAGAACTCCTATTCGTCCTTCAAAACCCATCACGGAA
ACCCCTCTTGGAGAAAACCCTCCTTCCTTCCCCTCAGGACTTTCCCAGCCACCGTCTCTCCTCCAGTCCA
GCCTGATGCCATGGGACTGGGGGTTTCTCTGTCCAGCTCTGTTTCTCCCAGACTGGGGTCTGAGGACTCT
CAGGACCCCCAACTTTACCTAGCACAGGCTGGGCACAAGTGGGTGACAGGGAGTCTACGCCTAGTGGAAT
TATGTATTGGGGCAGGGTCAGTGTGAGAATACACATCCGCATGCATGTCTGTCCATGTCTGTCCGTACCA
ACCTTCCCCTTCCACACGGACCTGGGCACATAGGAGGTGTCTGAGCCTGACACATGGGACAGAGAGTGGA
CATGGCTGAGACACGGACAGAGAAAAGACAAGGAGTCCAGGGGGCTGAAAGCCTTTTGAAATCAGGAAGT
TCCTGTATTGGCAGAACAAAGCCCAGAGAGGAGCAGGGCTTTCCTCAACGCCACCCAGCAAGTGGACACA
GAGCCCGGCCTTGGATGACACCTCCAGGGTTCTGAACCCTGGACCTCGCTTTATGCAAGGAGCTGGCCCC
ACATTTCCATGAATCGGGGAAACAGCACAAGAAGGTTGGCCTGTGGCAGGGCAAGGGTTAAAGGGGTGAC
ATTGAGGGATGCCTCAGAGTCAAAGTCCCCTGACCAAGAGGAATAGAGTAGAAAACACAGAGACAGAGGG
TGAGATCACGCCCCGATGAGGACGGAGAGAGACAGAGATGGAGAGAGACATAGAGGTGGAAATATACAGA
GAAAGATAAATGCAGAGACCAAGGCAGGGAGTGTCGGGGGAAGTAAAGAGGGTGTCCTGAAGAAAGAAGG
ATCTGTTCACTCTTACCAGTCTGTCCTCGAATGATTTGCATAAAATGAGGAGGTGCCTGTCCACACCCCC
AATTCCTCTCTCAGGCCCCAGAGCCTGAGACCTCACCATGCCCCCATCAGAGATGCAAAAAACTAAACAC
CCAACTAGAAATCCTTGGGACCTCTCTCGGCTGGGATCTCAGAGCCTTTCTGTCCCCTACCCCTACCCCA
TGTGCTGTCGATTTTGCAGATGGGGACAACCTGGGGCCTCCCGGAACTCTGCCACCCTGGGGAAGTTGGG
GGAGGGCCTTAGTCCCGGATCACAACCCCGTCTGCTCCCCAGAATCCTTTCCTAAGAATCGTTGAGGACC
AAAGTTGTCTTTGCTGACACGTGTTGCTTTTCTCTTTGCCTTTTATTGTTTCAGAGAAAAATCAAGTTGA
CTGTGTCAAGTAACACCCCACCCCTTACCCCCGTCCAGCCATAGTGGCTCTCTGGAGACACAGGTCACAG
GCGGAGGGTCCCCTGATCATCCCCAACCACACAGCCAGGGGGACTTGACCCCTGTCCACCCCTGTCTCGT
GCTCCCTCAGACCCCCACAAACCGGCCAAGCAGTCCGGGGAGGCTTCCCCTCCACACAACTCTTAGCATG
TGATTGCAGATGTGAAATCAAAACGTTGTTTGTTTTTTGTTTTGTTTTGATTCTACCCCGTCGGTCCAGT
GTCTGCACAGACGCCTTCATTTCTCTGTAAATATGTGACTTGGAACAAATGTTTAACACAAACGAGAAGT
GGTCATGAATGCATGGTGTTGAGATGTTTTGCACTATTCTGACTTTTTGGTCTCTGTAAAAATATTTTAT
TAACAGCAGACATTAAAAAAAGAAAAACCACACACAGCCTTGGA
(SEQ ID NO: 5063 (SOURCE NCBI Reference Sequence XM_011527811.2)).

In various embodiments, a UNC13A transcript is a pre-mRNA UNC13A transcript. In various embodiments, a UNC13A pre-mRNA transcript comprises a sequence provided as SEQ ID NO: 5064.

CAGTGGCTCACACCTGTAATCCCAGCACTTTGGGAGGCCGAGGTGGGTGGATCACTTGAGGTCAGGAGTT
GGAGACCAGCTTGGCCAACATGGTGAAACCCTGTCTCTACAAAAAATACAAAAATTAGCCGGGCATGATA
GAGAGTGTACCTGTAATCCCAGCTACTCGGGAGGCTGAGGCAGGACAATCCCTTGAATCTGCGAGACAGG
TTACAGTGAGCTGAGATCTCAACACTGCACTCCAGCCTGGGCGACAGAGCAAGACTCCATCTCAAAATGT
ACCCTGTAACAATATATAAGATGTCAACATTAGTAGAAGCACTCAAGCCTAGATGATAGAGAGAGATTCA
GTCTCAAAAAAAAAAAAAAAAAAAAAAAGAGAAAGAAATGGATAAAAGACTTGAACAGGCATTTCACCAT
AAAAAGAAACCAGTAATCACATAAAAAGTTCAACGTCAAGAGCAATCAGAAAGCAAACCAAAACCCAAAT
GAGGCACTCCTTCCCTCCCTCCACATAGGCCCAGTGAAAAAACTTGGACAATTCAAAGTGTTGGCAAGAA
CTTAGAGCCAGGCACGGTACCTCATGCCTGGAATTCCAGCACTTTGGCAGGCCGAGGCAGGAGGATCACT
GGAGCTCAGGAGTTTGAGAACAGCTTGGGCAACATAGGGAGACCCCATCTAAAAAAAATAAAATGAGCCA
GGCATGGTAGCACAGGCTTGTAGTCCCAGCTCCTTGGGAAGCTGAGACAGGAGGATCGCTTGAGCCCAGG
AGGTGGAGACTGCAGTGAGCTGTGATTGCACCATTGCACTCCAGCCTGGGCCTCAGAGTGAGACTATCTC
AAGAAAAAACAAAAACCAATAACAAAACAAAAACAAAACATAGAGGAACCGATGATTATACTGTGTTGGT
ATGATGAAAATCGCCAGACTCCCAATAAAAAACATGACATTATCAGGTGAAGCTGAGCATATATGTATTT
TCTTTTCTTTCTTTCCCTCCTTTTTTTTTTTTTTTTTTGAGACAGAGTCTTGCTCTGTTTCCAGGCTAGA
GTGCAGTGGCGTGATATCGGCTCACTGCAACTTCCGCCTCCTGGATTCAAGCAATTCTCCTGCCTCAGCC
TCCTGAGTAGCTGGGACTACAGGGGTGTGCCATAATGCCTGGCTATTTTTGTGTGTGTTTTTAGTAGAGA
CAGGGTTTCACCATGTTGGCCAGGATGGTCTCGATCTCTTGACCTCATGATCTGCCCATCTCAGCCTCCC
AAAATGCTGGGATTACAGGCATGAACCACCATGTCTGGCCACATATATGTATTTTCTGACCCAGCCATCC
TTACTCATGTGTGCCAGCACACAAGTCTGAAGTCTGAGGATGGTCATAAAAACTTTTTTTTGTAATAAAC
CTGAGCTTGAAACAACCCAAAAGTCCATGACCTGGAGAACAGATGAATAGCCAATAGAAGATTCATACAA
AGGAATACTATGCAGCAGTAAAAAATGCAAGCCTTACAGCCACATGCAGCGATATGGATGAATATCAGCA
GAATCACATTGAGCAAACAAAACAGATTAGCAGTCCAGGTGCTGTGGCTCACACCTGTAATCCCAGCACT
TTGGGAGGCTGAGGTGGGTGGATCACCTGAGGTCAGGAGTTTGAGACCAGCCTGGCCAACATGGTGAAAC
CCTGTCTCTACTAAAAATACAAAAATTATCACTGCAACCTCCATGGCCAGGCATAGTGGCTCATGCCTGT
AATCCCAGCATTTTGGGAGGCCGAGATGGGCAGATCACCTGAGGTCAGGAGTTCGAGACCAGCCTGACCA
ACATGGAGAAACCCCGTCTCTACTAAAAATTACAAAAAAATTAGCTGGGCATGGTGGCACATGCCTGTAA
TCCCAGCTACTCAGGAGGCTGAGGCAGGAGAATTGCTTGAACCTGGGAGGCGGAGGTTGCGGTGAGCTGA
GATCGTGCCATTGCACTCCAGCTTGGGCAACAAGAGCAAAACTCTGTCTCAAAAAAAAAAAAAAAAAAAA
AAAAAAAAAAAAAGCCAGGCAAGGTGATGGGCGCCTGTAATCCCAGCTACTCGGGAGGCTGAGGTAGGAG
AATCGCTTGAACCTGGGAGGCAGAGATTGCAGTGAGCGGAGATTGTGCCACTGCACTCCAGCCTGGGTGA
CAGAGTGCAGCTCTGTTTCAAAAAACAAACAAACAAACAGATTAGTAAAGAATGTGAACAAAGTCAAATG
AGGTGGCTCATGCCTGTAATTTCAGCACTTTGGGAGTCCAAGGCAGGCAGATTGTTTGAACCCCAGAGTT
CAAGACAAGCCTGGGCAACATAGTGAGACCCTCATCTCTAATTAGCCAGGCATGGAGGTGCATGCCTGTT
GTTCCAGGTACTTGGGAGGCTGAGACAGGAGGATTGTCTGAGCTCAGGAGCTCACACCACTGTACTACAG
CCTGGGTGACACAATGAGACCTTGTCTCAAAAAAATAGAAACATGCAAGAGGTAACCATGTATTGTTTGC
TAAGGAGCATGTGGTAAACTATAAGGAAAAGGAAGAAAATGAAAAACAAAATTTGTGATAGTTCTTCCAT
CTGCAGGGAAGGATTATGAGATTGGGGAGAGGCACATGGGGGTCTTTTATTGTGATGGTATTTTTTTTTT
TTTTTGAGACGGAGTCTCTCTCTGTCACCCAGGCTGGAGTACAGTGGCCCGATCTCGGCTCACTGCAAGC
TCCGCCTCCCGGATTCACGCCTTTCTCCTGCCTCAGCCTCCTGAGTAGCTGGGAATACAGGCGCCCGCCA
CCACGCCCGGCTAATTTTTTGTATTTTTAGTAGAGACGGGTTTTACCGTGTTAGCCAGGATGGTCTCGAT
CTCCTGACCTTGTGATCACCCCGCCTCAGCCTCCCAAAGTGCTGGTATTATAGGCGTGAGCCACCGAGCC
CGGTCGATGGTAATTTTTTTTATCCAAGCCTGCTCCTCATAGTTTATCTACTCATTGATGTTTAATAAAA
CATCAATGTTTTATTGATTATCAATGTTTAATAAAACATCTACTCATTGATGTTTAATAAAAATTTAAGA
CCAGGCATGGTGGCTCATGAGCCTGTAATCCCAACATTTTGGGAGGCTGAGACGGGAGGATCACTTGAGC
CCAGGAGTTTGAGACAGCCTGTGCGACATGGTGAAACTCTGTCTCTACAAAAAATACAAAAATTAGCTGT
GCTGATTTTTTTAAAAATTTAAAGCAGTGACAGATACAAACATGGTTACTATTAGGCATTAAGATTACAA
CTGGGGCCAGGCGCGGTGGCTCATTCGTGTAATCCCAGCATTTTGGGAGGCCACGGTGGGTGGATCACTT
GAGGTCAGGAGTTTGAGACCAGCCTGGCCAACATGGTGAAACCCCATCTCTACAAAAAATACAAAAAAAT
TAGCTGGATGTGGTGGCGAGCGCCTGTAATCCCAGCTACTCAGGAGGCTGAGGCAGGAAGATCACTTGGA
GGTTGAGGTTGCAGTGAACTGAGATGGTGCCACTGCACTCCAGCCTGGGCCACAGATTGAGACTACACCT
CCAAAAAAAAAAAAATTACAACTGGTTTCACATGTTAACTCATGTAATACCTCCAGCAACCTTGTGAGGC
AGGACTTGTTATCATATTCATTCTACAGGTGCGGAAACTGAGGCACGAGGAGGTCACAAGACCTGCCCAA
GGCCATGCTTGTGATGAGAGACAGAGTTTGGTTTTGAACAATGGCCTCACTCAACTCCATGCACATTCTG
CATTTATGAAGCTCTTTCATGTGGGCTGGGGGCAGGAGACCCTGTGGGGCTCAGCCTTTCATGCTAGTTT
AGGTGCCAGTGATACAGCGAGCAGGTTGCAGGGCAAGGGGCCCACATGGAGCTGCCCAGTTTGATATGTG
GGGAGGGGGCCAGGCGCAGTGGCTCAGGTCTGAATCCCAGCACTTTGGGAGGCCGAGATGGGCGGATCCC
CTGAGGTCAAGTGTTCAAGACCAGGCTGGCCAACATGGTGAAACCTCGTCTCTACTAAAAATAAAAAAAA
TAAAATAAAAAAAAAAAGCTGGGCATGGTGGCATGCGCCTGTAGTCCCAGCTACTCGGGGAGGCTGAGGC
AGGAGAATCTCTTGATCCCAGGAGGCAGAGGTTGCAGTGAGCCAAGATTGCGTCACTGCATTCCAGCCTG
GGCTGCAGAGGGAGACTCCATCTAAAAAAAAGAAAAGTGGGGAGGAGTGGCAGAGTTTCGTTCGGGTCAG
GGCCCAATCAGAGGAAGGGATAGGGGCTGGGGGAGTCCAGAGAGGAGAAACAAAGGCTCAGCTTGGGGCA
GTCAGGGAAGCCTTCCTGGAGGAGAGGATATTCAGACAAGACATTGATGGATGAAAAGGAGTTCAGAGTG
TGGGCAAGGACCCATGGGCTGGATGGCTTAGAGACATAGAGAGAGACCTAGACAGGATGCAGTAGAGCTG
CAAAAAGACTTTACAGAGAGAGGCTAAGAGACACATGACTGAGAGAGCAGTGGACAGAGTCAGAGGAACA
GAGACCCAGGGAGAGAAAGAGACCAGGCAGTAGAGTTAGAGAGACAGAGACGTAGAGACAGAGGAAGAGA
GATACTCAGAGAACAGGACCATCTGCCCGGTGCTGGTGCCGGGCGTACACACAAGAGCTTGCATTTGCCT
GTACTTGCTTGAGTATTGCAGGGAGGCGTTTGCCCAGGTTACCAGGGCAGGAATGTCCATGCATGGGTGG
CGGGGGCACGTGCACCATTTCAGTGCAGGTGAGGGTCTGCACAGGAGGAAACCTGCTTGGGGGCCGCCGT
CGCCCCTTGTCCCCCTGCAACGAGGAGGTGTGGGGCGAGGCGCCCCTTCCCGGAGCCAGGCCCTTTAACG
CCCCCCCAGCCCGCAGCGCCCCCGCCCCATCAGGCGTCCCTGGGCTCCGCCTGCCCGCCCCGCATCAGCG
CCACGTCAGGCGCTGTCCGCGCCCCCGGTGCTGAACCAAGATGGCCGGTGGCGGCCGGGCCCCGGCGTGA
GCCAAGCGCGGGCTGCAGCCGGGAGATGCCCCAGCCCAGCGGCCGCTGAGCCCGACCCGACAGAGCCGGC
CCGGCCGCCTCCGGCCCACCTGCGAGCTCGGAGACATGTCTCTGCTTTGCGTTGGAGGTAGGAGGCGAGG
CTGGGGGTCGGGGGACCCGTGTGGACGCGGGCTGGGGTCGGGGTCCGCGTGGATGCGGCTCCCAGGGGGT
TCCTGGGGGGTGACCCGAGTCCAGGTGGCTGGACTTTTGAGAGGGGTAGCCCTTGTCGACGCAGCTGGGC
CCCGGAGGGGGTGATCCGTGTAGACGCGGCAGGGCTCTCGGGGGTCCTGGTGGCCGCTGCTGGAGGGGTC
GTGTGGACGCGGCTGAACGCGGAGGCCCGTGTGGACGTGGACGGGTATCCGGGGTTCTACCCGGAAACGA
TCTAGACTGGGGCGTCTGTGTAGACCAGGTTTTTAGAGGGTCAGTGAGGAAGAGTCTGGGCTCGGTAGTT
TGATTGAAGCCTGCAGGGGTGCTTGGAAGGGGTCTGGTGTTGTGGGCTGGGTTACGGGTCCTCATGGACG
CGGACCCGGCTGGAAATTCGAAGGAGAGTGCGGTGGGAGGGTTCTCCAAGCCGTGGGGATGTAAGATATA
GGTAGAGGGGCTTGGCCATCAGGGCATCCACGAGTTCTAGGACTCTCCCAGCCCTAGGTGTAAAGAGGAC
TGGAGTGGGGGGAGTGTGGAGACAGGAGACCCCTCCCAGCGTCCAGAGAGGGCTGTGACTGGAGGTCTGG
GGGCGCTTTTCCAGTTAACTCTTTGGCCTCCCGAGTGAGGGGGTTGGAGGAATGTCCAGAAGGTCTTCCC
GCTCCCTGCCCCCTCCGGTTCTCCCAAACACGTTTCAGGGCCTTCTCGGCAGGGAGCCTACTTCTTGGAA
GAGCAATGTCTCCTGGTCCCCCTCCTGGCCCATCCTCACCTCACCCACTTTGCACTGGAGAGAGGGAGGG
AGGGAGACGTGAGGTGGGGGGGGAGCTATTTTGGGGCCAGAACGCCATCTGCACAGAGGCCCACGCCCAG
GCCCACGCTCTGCGTTGGGGTTGGAGGAGGGAGGGAGACGTGGGAGGGGCATGACCCCCCACCCCACACC
TTTATGGCTCTGGGGTCGTGGGGAAGCCAGCCCAACCGAACCCAGGCGTTCTGGGCACTCCCGTTTGAGA
AATCAAACTCTGGGAGACCTTGGGCGGCAGGGGTGGATGTGGACCGCCGTTCCACGCCTACAGAGCTCTG
AACCCCTGGTTCTTAAGGACTGGGGTCCCCCCGTCACTCCCACCCATTTCAAGAAGGCTGGGGTGGGGGA
AGGGGTGTGTCTGCCAAGCCTGAGACTGCAGGCTTCGGGAGACCTCGGGCGACAGGAAGCGTGCGTGTGC
TGAACTCCTGCTGCCTGGACACAGGGTGGGCAGGCCGGGGAGCCGGGAACCGCCCCCGCCGCCCCTCCTC
CTCCCTCCCCTCCCCCTCGGCCGATTCATTCACAAACCCTGCCCCGCCCGCCCGCTCCCCGCCGCGGCAG
CTTCCGAGTGCAAATCCGGCCGGGCTGGGCCGGCGTGGACGCGGGTGTTCTTAAAGGGACAGGGGCCTAC
TTTCTCTAAGCTCGTTTCTGCTGGACGGGGCTGGGGGTGGTGGAAGAAGGGTCCCTAGAGTCAAAGGTCA
ACTCCTCCATCCCCTAGCCTCCAGGCCTATGGAGTTAGGGGAGTAGGGAAGTGAAGGAGACCCCTAGGCG
ACCCCCACCGCCTAGGCCTGAGGACGGAATGGGAGGAGAACTTGAATGTCCAGACCCCCTACTGACCTCT
CCCCCTGCTCTAAGTAGCCGGGCTCTTAAAGGTGGCACTGGAGGAGGTTCCGACGTGGGTGTTCTTGGCT
GACTCCTCTATCCTCTCTCTTCTCCCCCCAGCATCCCGCCCCACCCCGCCTCACATCCTCCAAGTTGGGC
TTCTCTGGAAGGTTTGCCTAGTGGAGTGGGGGGGGGGGGGGGGGGGGGGGGATCTCACCCTTAACTCTGA
CGCAAACAGGAGGCTCGGCTGCGGGGGTGGCATTAAGGGGGGGCAGCAGGAGGCAAGAGGGATGAGGCTT
CCAGAAACAGAGACGTTGGGGGAGGACTGTGCTGGATTCTGTGGACTTTACAGGGAGGGGCCCGGCTGAG
TGGAGAGGAATCGCTGGGAGGGGCAAGGCTGGGGAAGAGGTTGACAGGGGTGGGAGGTAAGGCCCTCAGG
TGTAAAGGGAGGTGTGAGGTTGGAGTGAGGAGGAGTAGGAGGCAGGGAGGGGTGGGGAGAGGGAGAAGTG
CTGGGTGAGGCCAGTCTCCCAGGGAGAGGTGGGATGGTGCCCAGAGAGGGGAGGGGCTGGGATGGCTGAG
CACGAGAGATGTTGGGGTGGGCTTGGCAGGAGTGAAGCTAGGCTGAGATGGGTTGCCGTGTTGCCTGAGC
TCAGCTGAGGAAATACCTGTCCCCCCTCCCCTCCACCGGGGACCCACAGGCGTTTGCCAATCCCTACACA
CATTCATACCCAGACTTGTTCCTGTGTGGACACAAATGCTTAGGATGTATGAAGGCTCTACGCAGACCAT
GTGCCTGGGAAGGCACATTCCGTAGGCACACGTGTGTACTCAAATGCCTGTGTGAACACAAGCATACACG
TGTATATAGGACACAGTCTGCCGCTACAGATATAGTCCCAGCAACACACGTGTGAGTGTGTGTGTCTGCC
AGCACGCATATAGATGTATCCACGTGTCTGATGAATGCTCACAATATACATGTCACATAAAGATCCACCC
ATGTCCAGCCGAGCGCGGTGGCTCAGGCCTGTAATCTCATCACTTTGACTTTGGGAGGCCAAGGTGGGCA
GATCACTTGAGGTCAGGAGTTCAAGACCAGCCTGGCCAACATGGTGAAACTTTGTCACTGCTAAAAAATA
AAAAAAATTAGCCAGGTGTGGTGGCGCGCACCTGTAGTCTCAGCTACTCGGGAGGCTGAGGAAGGAGAAT
CACTTGAATAGGGGAGGTGGAGGTTGCAGTGAGCTGAGATCCTGCCACTGTACTCCAGCCTGGGCAACAC
AGTGAGACTCTGTCTCAAAAAAAAAGATTCACTCATGTCCTAACACTCAGACATGTACCCATACATATCT
GTTAAGGTCCCCAACCCCATGAAGCACATATATATTCATATAAGCACATGTCAAAGCCATGTGTGTATGC
ACATGACAACACTTGCCTGGACATTCAAAGCAGCCCCTTTCAACAAGTGGATGATGATGCATACAAGTAA
ATACACATTTGAGTATTTCATTGCACACAAGTGCATACATGTATGCAGATAAACAAATACAGCTGCCACA
GAACTGCATCCATGTACACATGCAAACATCAATATGTGTGTTTTCCACCTAACATATCACCCACAAGCTC
ACACACACCAGTGTGTCTCTGATGTAGTGACACCCCACTCCTAGACACACATGTGCAGACTCCCACCTGC
ACTTAAATGGTGCACATACTCTGCACACATGCACACTCATATCCATGTAGAGGCAGGCACCTGCCTGCCC
TGGTAAGATGAGCCAACTACTGCTGCAAATACCCAAGCAAGTACAGGCAAATGCAAGCTCTTGTGTATAC
ACCCAGCACCAACACATAGCAGACCACCCTGTATGGTGTATGGGCCTTATCTCTGCCCGCTTCATTTATT
TATTCATGTGGTGCCCCGCTGCTTCCCAACATCGTAATCCACTGACTAGGTCTTGGCAGGATCCTGAGGC
TGTGAATGGTAAATGCCACCTGGTTCCTTCATGAAGTCACTTATTTTATTTATGCAACAAGCATTAATTG
AGCACCTACTGTATACCAGGCACTGTTCCTGGCTCTGGGACCCAGCCCTATTCCTAGCCATCACTCCTGC
ACGTCCCATGGGGTTAGAATGAAAGAGATGAGGGAGGGCTAAGGGAGCTGAGGAGTGGGGTATCCAGGGC
TCTCTTCCTCTGGGAAGCCTACCCTGATTCCACCACCACCATTTACTTGAGTCTCAGAAGATGTGATGAG
GGGGAGATGAGCAGGAGGGAGATGGTGCTCTGGGTGGAGGGAACAGCATGAGCAAAAGCATGGTGGTGTG
AAGGGTTACTTCCAGATCTTCCTATGTGCCTCACAGCATTTTGCACAGGCCACTTGATGTTTTGTTTTGT
TTTGAGACAGAGTCTCACTCAAAGCCTGTTACTCAGGCTGCAGTGCAGTGGCATGATCTCAGCTTGCTGC
AGCCTTGACCTCCAGGGCTCAAGTGATCCTCCTGCCTCAGCCTCCCAAGTAGCTGGGAGTATAGGCATGC
ACCACCACACCCGGCTGACTTTTTTCTTTTTAATTTTTTGCAGAAGCAGGGTTTTGCCCTGCTGCCCAGG
CTGGTCTTGAACTCCTGGGCTCAAGTGATTCACCCTCCTCAGCCTCCCAAAGTGCTAGGATTACAGGTGT
GAGCCACCACGCCCAACTGATGTTTTAAAGGCTCTATAAACATGTTATGAATAAATGACATGAACAAATC
AGTGAATTTAGGTGAGGGTGGGTTTGCTTCACAACTTACAATAATATCAACAACAAACCCCATTTATCCA
GCTTTTTTGTTTGTTTGTTTGTTTTTTGAGACACGGTCTCACTCTGTCACCCGGACTGCAGTGTAGTGGT
GTGATCTCGGTTCACTGAAACCTCCACCTTCCAAGTTCAAGCGATTTTCCTGCCTCAGCCTCCCGAGTAG
CTGGGATTACAGGCACGTGCTACCACACCTAGCTAGTTTTTGTATTTTTAGTAGAGATGGGGTTTCTCAT
CTCTACTAAAAATACAAATACATCTCTAGTGGCCAGGCTGGTCTTGAACTCCTGACTTCAAGTGATCCTC
TTGCCTCGGCCTCCCAATGTACTGAGATTACAGGCTCGAGCCACCGTGCCCAGCCCAGCATTTCATAGGC
ATGATGTTAAGGATTTTTATGCCCATGCCCTCTTTAACTCTTACAGTAGCCCCATTGTACAGATGAGGAA
AACTGAGGCTCAGGGAGCTTTAACAACTTGCCTGGTGGAACAGAGCCAGAGGTGGAGCTGACTGACTCTA
GAGCCTACTGGGGTTGGCAGAAGTTTGAGCCTTCTCTCAGACCCCAGGACCTCGGAGACAGCCAAGTCCC
TTCTGTGGAGTGATGTTCTGCCCTGGGATTGCCTTCTTCTTTCTATTTATTTATTTATTGGAGACAGAGT
CTGGCTCTGTTGCCCAAGCTGGAGTGCAGTGGCACAATTTCAGTTCACTGTAACCTCCTCCTCCCGGGTT
CAAGCAATTCTCCTGCCTCAGCCTCCTGAGTAGCTGAGATTACAGATGCACACCATCATGCCCAGCTAAT
TTTTTGTATTTTTAGTGGAGATGGGGTTTCACCATGCTAGCCAGGCTGGCCTCGAACTCCTGACCACCAA
GTGATCTACCTGCTTTGGCCTCCCAAAGTGCTGGGATTACAGGCATGAGCCACTGTGCCCAGCCAGGATT
GCTTTCTTCTCTCATGCATCTAGTAAACAGACATGTCACAATCTCTCCATTCATCGGGGCTATTCCCCTG
CTCCTTGCCTAAGAAGCCTTCACACATCCCTCCGTTCCTGGCTACTCAGTCCCTGATACCCTCCAGCTAG
GAGGAGCTGCATCTAGGTCTTAGCTCCTACCGTTCTCATGGGATGCCCTCCAGAGTTGCATCTTCAGGGC
TGCAGCCTAACCCTTTCCCTAACCTGGCCTCACTTTTCTTCCACATACAATGGACCTCTTCTGCAAGGTG
CCAATAAGGGCATCCTGGGTCCAGCAGGCCTGAGTGTGAATTGCTAAGTCCACCACTTCCTGGCAAGTCT
CACATCCTCTGCATTCCTTGGTGTGCTCATCTGTGGAATGGGCACAGGAGTAGCATCCACCATCTACGTT
TGCCAGAACTCTCTAATGAGGCTTGCAGAGCCCCTGGCATGACTCCTGGCCTACGGGAAGGCCCAATAAT
CGTGAGCAAAAGACATGAAAAATCAATGATGGGCCGGCCCCGGTGGCTCACGCCTGTAATCCCAGCACTT
TGGGAGGCCTAGGCGGGCAGATCTCCTGAGCTCAGGAGTTCAAGACCACCCTGGGCAACATGGTGAAACC
CTGTCTTTACTAAAATACAAAAAAATTAGCTGGGTATGGTGGCGCGCGCCTGTAGTCCCAGCTACTCAGG
GGGCTGAGGCACGAGAATCGCTTAAGCCCCGAAGGCGGAGGTTGCAGTGAGCCGAGGTCGCGCCACTGCA
CTCCAGCTTGGGCTACAGAGTGAGACTCCGTCAAAAAAAAAAAAAAAAAAAAATCAATGATGATGTTTTT
ATTAATGGAGACTAAAAGCTTTCTGGAAGGCGGTTCTGAGTGCAGGACATTTCTGAGAGTCCACTGCATT
CTGGGGCCCGAGGAGCGCTCCAAGAGGGGTCCCCCAGGGCCACTCCAGGAGGGAGCCTAGTTCGTTGAGG
GTACACGGGTGGGACCGAGCATTCAGCACCGTGGACAGCGCCTGCCCGCGCCTCTGGGGGACAGCTCTTA
GCTCAGCCTCCCAGGATTAATGGAGGAGGTGACGTCATTCCTTCCGTGAATTACTATTTAATGAGCACCT
ACTATAGGCTAGGCCTACTTCTAAGTGGTTCATGGTAAACAACACACACACAAATCCCTTATTCCCGAAG
CTCTCTGGAACTCCAGGTTCTTGGTGTTTTTTACACTGAAGGCTCAGTCTGGCTCTGGTCTATTTGTGTC
AACGGAGGATAAGGGTCCTCATTTAGTGTCTCCCAGATGCGAAACCAGCACAGCTGAGGTCTTTTATCCT
AAAGCCCATCCATCTCCCGCTGAAATGCCCTCCACCAACATCACACACAAAACGATCAAATCTCCTATAA
TCTATTTCTGGATTCTCTATGCTTCCTCCCTTCCCCCAAGCTAATCCTGGGGGGGGTGGGCTTTCAGAAG
CCCTAGACGGCTGGATCTCCACCTCCTGTCTCCCCACCCCATCCCACGTGGCAGGTCAGGGAGCCAGTGC
CCCAAGACTGCAGTCCCCCCTAATTAGAATCCAGGGTCAGTCAGCCATGTGGTTCACGCCTGTAATTCCA
ACACTTTGGGAGGCCGAGGGGATGGCTTGAGCCTTGAAGCCAGCCATAGCAACATAGCAAGACCCCATCT
CTACAAAAAAAAAAAAAAAAAAAAAGGAATAGCCAAGCATGGTGATGCGCACCTGCAGTCTCAGCTACTC
GAGAGGCTGAGTGGGGAGGATCGCTTGAACCTGGGAGATCGAGGCTGCAGTGAGCCATGATCACACCACT
GCCCTCCAGCCTGGAGCCTGGGCAACAGAGAGAGACCCTGTCAAAAAAAAAAAAAAAAAAAAAAAGAAAA
GAAAAGAAAAAAAAAGAAGAAGAAGAAGAGAAAAGAAAGAAAAAGAAAAGAATCCAGGGTATTCAAATCC
TAGAATCCAGGCCAGGGACACCTTTAGAACACAACCAAATAATAAAAACAGTAAAGATATCAGTAATAAT
AATAATAGTATAAATGACACTATAATCATTCTGCAGAAGGGGTGAGGGAGGAAGAGGAGGGAAGAAGAGG
AGGAGAAAGGAGAGGGAAGGGAGATCAGCCACTCCGTCTCCCCATTTTCCAGATGCAGAAACGGAGGCGC
GAAGCCAGGCTGCAGGGGTGTCCCGCAGCCTGAACTCCTGCCTCAGCCTCCCCGGGTACGGCCACTACAT
TCATCAGTCAGCGCTTCTGGGGGCCCCAAAGACCTTTGAGGCCGCATGACGCGGTGTCTTCACCCGCTCG
TCGCTGGGGACAGCAGTGGGCACCCCCCTCATCCCCGCCCCCCTGTCCGCCGGCGCGCCTCCCACGCCCT
CCGCGCGCCCACGCTATAAATAGCCTCATGCGTGTGACCCGAAGCCCGCTGGGGAAGGGTGGCGCTTCCG
CCCCGCTGACCCTGATACAGCAACCCGGCGCAGGCGGGGAGGTGGCGGGATGTGACGACGAAGACGGTGA
CTCAGGGACCATTTACTGACCGGGGAAGACAGGGAGAGAGACTGGGTTCCTTCTCTGAGCCCCCTTCCTC
CTCCAAGCCTCTCCCCCGATACCACCCTCCCCTCTGCGCCCATCCCCCTTCCCGAATCCCCTCCCTCACC
GCGAACACCCTTCCCTCCTGAGAACCCCCTCGCCACTCTCGGCCCCCTTTCCTGTCCCCTTTTCCCTCTG
AGACCCCTCTCCCACTGGGAGCCCCAGTGATGAAAAATCATGGATGTCCTCAACCTGGACCTCGGCAGGG
AGCTAAGTGCAGGAGCCTGGCTTCAGCTATCAGAGGGTACAGCCCTCCAGCCGGTCCCCCACTGCCTGAG
GACCCCAACGGAGACCATTCCCCTCTAGCCAAGAGAGGCTGCAGGAAGGAGCTGAGGCTTAGGGAAGAGG
AGCCGGACTGACCAAGGGAGGGGCACAAGAAGGGCAGAGACAGTGAGAGAGAGAAGGAGAGAACAGAGAC
CAAAAGAAGACGACCAGAGTGGAGAGAAATAGAACGGGGAGGAGGGAGATGAGAGACAGGGATTGGGAAA
GAGAGAGAGAGAACAGATCAAGGAGCTACTGGGCTCCCCGCTGAGCCTAAATCCATGGTTTATAAAACAG
GGCACAGTTGGAGGAGCTTCAAGTGAGCCCAGCCCAGTCTAGACCCCAGTGAGCCCCTTGTATCACCCCC
CAGAGCCTGTCACTTTCCCACCCCCATATGCATCCCTACTTTTCTCCCCCAACTGCCTCTCCTCTCCCCA
GTGTCTCCTCCACCTGCCTGCCCTCCCTAGTGGAAATCTTTCCCCCTCTCTCTTTTATTATTATTATTAT
TATTATATATTTTTTGAGACAGAGTCTTGCTCTGTCGCCCAGGCTGGAGTGCAATGGCGTGATCTTGGCT
CACTGCAACCTCCGCCTCCTGGGTTCAAGCGATTCTCCTGCCTCAGCCTCCCGAGTAGCTGGGACTACAG
GCGTGCACCATCATGCCCGGCTAATTTTTGTATTTTTAATAGAGATGGGGTTTTACCATGTTGCCCAGGC
TGGTCTCGAACTCCTGACCTCATGTGATCCACCTGCCTCGGCCTCAAAGTGCTGGGATTACAGATGTGGG
CTACCACGCCCAGCCTCTCTCCCTTTTAAATTATTATTTTTAGACATGGGATCTTACTACATTGCCCAGG
CTGGTCTTGAACTCCTGGGCTCAAGGGATCCTCCTGCCTTAGCCTTCCTAGTAGCTGGGATTACAGGCAT
GTGCCTCTATGCCTGGTACTCTCGCTGTCTCCTTGTGTCTCTCTATTTCTCTCCATCTGTCCAGACTCTC
CTCTCTTGCTTTCATGCCCATCATTTCTTCCTCTCTCCTCTCCCTTCCTCTCCCTGTATGAGTCCCTCCA
GCTGTGGGTGCCTTTCTCAGCCTCTGTCAGCATCCCCTCCACCCCCGATCCCGTCACATTCTACCTCTTG
CCCCTTCCCCTGCCCACCCCCTTCACAGAGAGTCCCCACGCCTGCAGGCTGGACCTCCTCCAGGAGCCAG
GCCCAAAGCTCCTGCTGGGCACATTCCTGAGTGTTCCTGAGCCTCTTTCAGTCCCAGGGGTGAGGTGCTG
GGTGGGTGCGCTCCCTGGGGGCTGGGTGGGGCTGAGGATGTGAGCGAACAAACCAGACCTAGAAAAAAAA
AAGACAGAGATGGAAGCAGAGATGAAGGCCCAGAGAGAGGGGTTCACTTGCCCAGGGTCACACAGCTCAG
GAATCTGAACCTGGCTCAAACCAGCATTCACTTCTCTCAAGGCCCACCCAAGGGCTGCAGTGCAGTGGCA
CAATCTCAGCTCACTGCAACCTCTGCCTTCCAGGTTAAAGGGATTCTCCTGCCTCAGCCTCCCGAGTAGC
TGGGATTAGAGGTGCGTGCCACCACACCCGGCTAATTTTTGTTATTTTAGGAGAGACGAGGTTTCCCCAT
GTTGTTCAGGCTGGTCTCGAACTCCTGACGTCAAGTGATCCACCTGCCTCGGCCTCCCAAAGTGCTGGGA
TTACAGGCATGAGCCACCGCGCCTGGCCAGGAACAGTCTTTACTGACATGTGTTTAGGGCCATCGCACTT
CCCCCGCTCTTCACGGGAATTCTCTCTTCACTGGCTCCACTCAGGTGCCTTTTGTGAGACAGAAATTCAA
GCGATGAGGACAGAGATACAAACCCGGGATGGGAGGCTGTGACATCTGCCCATAGAAACACAATTCTGTT
TCTAGGAATTTATCCTTCAACTCGCTAGTACACGCTCTTCAAGACATACTGAAAAAACATGAAGGATTAG
AACAGTGGGTGTCGAATGCTACCATTTGTCTTGGAAATCAAGCAAAACGAACGTGGGTATGGTTGCCGAC
GCATGGACCATCTCCAGTGGAACAAGGAACTGCCAACGTTGATTGCATTTGGGAGGGAGATGAAGGGCTG
GGATGGAAAGAGCTTTATTCTCAGTGCCTCATCTTTTGGTTGTTTTGGATTTTACAACATGTGCATGCAT
TGCCTACACAAACAAAGACACTAATTTGAAAAGGAAAAGGGGAGAAGAAAAGAGGGAGGAAAGAAAAGGG
GCCGGTGTGGCAGCTCACACCTGTCATCCCAGCACCTTGGGAGGCCAAGGCGGATGGATCACCTGAGATC
AGGAGTTTGAGACCAGCCTGGCCGACATATACTGAAACCCTGTCTCTACTAAAAAATACAAAAATTAGCT
GGGCATGGTGGCGCACGCCTGTAGTCCCAGCTACTTGGGAAGCTGAGGCAGGAGAATTGCTTGAATCTGG
GAGGTAGAAGTTGCGGTGAGCTGAGATGGCACCACTGCACTCCAGCCTGGGCAATAGAGCAAGACTCCGT
CCCTAAAAAAAAAAAGAAAAAAAAAAAGAAAAGAAAGAAAGGAAAGGGAGGGAAGAGCTCAGTGTGGGAG
AAGATTGGGGGTGGGAGTAGAGAATTAATGGAGGAAAAAGATGTGGGGGACAAAAGCAAGGTGTGAGAGG
TGTTGGCAATGGAAACTGAATACCTGTTGAGAAGAGCCAGGAGGGGGCGACATGGCTTTGAATCTCTTGG
GGCTGTGAGATTATGTGAAATGTGAGAAGACTGGACCTCAGTGGGGTGCCTGACATATATTCAGGTTACA
CCTCCCACAAGCAGGTGGGGCTGGACAATCTCTGTCAACCCTCCAGCCAGGACCCAAGTGTCTCCCTGTA
CATTACTTTTGGTTGTTGTTGAGACAAGAATCTCACTCTGCTGCCCAGGCTGGAGTGCAGTGGCATGATC
TTGGCTCACTGCAATCTCTGCCTCCTGGGTTCAAGTGATTCTCCTACCTCAGCCTCCCAAGTAGCTGAGA
CTATAGGCGCCTGCCACCACACCTGGCAAATTTTTGTATTTTTAGTAGAGACAAGGATTCACCATGTTGG
CCAGGCTGGTCTTGAACTCCTGACCTCAGGTGATCCACCTGCTTCAGCCACCTAAAGTGCTGGGGTTATA
GGTGTGAGCCACCGTGCCTCGCCCTCCACATGCATTTTGAGAGCCAGCTCTCTGGGTTGCTTTGTCCCAA
CTCACCCAGCCTTCTTTGAGGCTTCCCCAAACCCTGGGAGCATTTGACATTGCAGACTGAGCTGGACAAT
CAAGGGAGAGGTCTTGCTGACCTGTCGGGGCTGGTGGAGTCATCAGAGATTGTCTTTATCCTCGCCTTCT
TCTTCTCCACCTCCTCAACTTCCCCTCTTCTTCTGGGGCAATCCTGGAGGGCTTCTTGTAGGAGGCCTCT
CCCACCATCTGCCCAGCTCTTTCTTTCTAATCTTGTTCAATTGTGGGTAAAGGCACAGCTAGTTGGGAGC
TTGGGGAGTTTGATGGGGACAGGGGATGCAGGGAGAGGGAGGGGCAGGGAATGCTCTCAGGGTCCAAATT
TCCTTCCATTCTAGGGTTGCCTGTGCTCATCTGTCTGATGGCTTCTCTCTCTCCCTCTCATCCCTGCCTC
CTCTTTCCTACCTCTCCTCCTCCCCTTGTCTCTGTCTCTGACATTGCTTCTATCTTTCTTTTTTAACATC
TGTCTCTTTTGCTTTTTATCTTTGCCTGTCTCTCCTCTGTGTCTCTCTCCTTATGTCTCCATCTTGGTGT
GTCTCTGAATCTCTCCGTATCTCTGCCTCCTCCTCCCCACATCTCTCCCCACCTCCCTTCCCTATCTCTG
TCTCTCTCCACAGTCAAAAAAGCCAAGTTTGATGGTGCCCAAGGTAAGTGGCTTCTCTTGCTGTCCTGTC
CCGTGTTGTCTGTCTGTCTGTCTGTCTCTTGGAGAGACTGGGAGGGGGTCCCAGCCCAGACTTAGAGTTG
GGCTTTATGTCTTCTGAGCATCAGGAGAGGGGTCAGAAGATTAAATAGGGGGAGGGGCAGCCATTGGCCT
GGGGTCCCTGCAGTTTGTATGGGGGTCCCCGGGTCTCCCCAGCCATCCTTGGAGAGTCTGTAAGGGGTGG
GGCTTGCCCTGAGCCAGTGGCTCTTCCCAGGGTCCTAAGAGGTAGAATGGGGACTCTTAGATGGGGCTAC
AGGTGGGAAAAATGAGGCTCAGAGAGGCGAAAGGAGTTTCTTTCTTTTCTTCTTCTTCTTTTTTTTTTTT
TTTTGAGACAGAGTCTCCTTCTGTTGCCCAGGCTGGAGTGCAGTGGCGTGATCTCGGCTCACTGCAACCT
CTGCCTCTGGGTTCAAGCAATTCTCCTGCCTCAGCCTCCCAAGTAGCTGGGATTACAGGCGCCCTCTACC
ACGCCCGGCTAATTTTTTTATTTTTAGTAGAGATGGGGTTTCGCCATGTTGGCCAGGCTGGTCTCAAACT
TCTGACCTTAAATGATCCGCCCGCCTCGGCCTCCCAAAGTGCTAGGATTGCAGGCGTGAGCTACCGTGCC
CTGCCTTTTTATTATTATTATTTTTATTTAAAATTTTTATTTTTTGAGGCTGGAGTGCCGTGGTGCAATC
ATGTTTTACTACAGCCTCAACCTCCCAGGCTCAAGCAATCCTCCCACCTCAGCCTTCCAAGTAGCTGGGA
CCACAGGCGTGCACCACTGCACCCAGCTAATTATTTTTTTGTATTTTTTAGTAGAGATAGGGTTTTGCCA
AGTTGCCCAGGCTGGTCTTGAACTGCTGGGCTCAAGCAATCCTCCCCCCTTGACCTCCCAAAGTGCTGGC
ATTATAGGCGCGAGCCACTGCACCCAGCCTGCAAAGGGGGTTTCTTACAGTCAATTTTGTTCCCCTTTTC
TGGGACCTCAGTCTTTTTAGCTGAAAACCAATGGCATGGTCATTTCGAAGCAGCTGGATGAAGTATCCCA
AAATTCCACGTCTCCCCACACTGAGGCCCCTCCCTTGTGCCCTGAGGGTGGGGTTGTGGGTTAGGGGGTG
AGTGGCCCAGAAATTCCTGAGGGGATCACAGCAGATGGTCTAGAGCTTGGGAAGGGGCATTGGAGAGTCC
CTGAGCCCCAGCGCTGACCCCTACTCTCACTGCCACAGAGAAATTCAACACGTACGTGACCCTGAAAGTG
CAGAATGTCAAGAGCACGACCATCGCGGTGCGGGGCAGCCAGCCCAGCTGGGAGCAGGATTTCATGTTGT
GAGTCTGCAGCGTCTGGCATGACCTCACTCCCTGGCATAGCCCAGCACCTCGCTGGCCCCTCAGGGCAGA
GCTGGACACTGGGGAATCCCGGGTGACTCCCCTCTGTCCTCCCTCTTCCCCTGTCCACCTTATCACCTTC
ACCTCCAAAACACATCCCCAATCCAATCACTTCCCTTGGTCTCCGCAGCCACCAGCCAAGTCCCTGCTGC
CCGCCAGCCTCCGCCCTGGCCTCCTGTGCCATCCACCTCCTACAATCTGTTCTCCTCCAGGCAGCCAGGG
GGATATTTTTAAAACTGCAAATCACATCCTGTCGCTCCCATGTTCCAAACTTGTCCGTGGCTCATCGACC
TCACAATAAAACCCACAGCCTTCCCCGTGATTCCCACCAGGTTCTCGCTCTATCTCCCTCACCTGCCCCA
GCCACACCCACCTCTGCCCCTTTCTCCAACACTTCCCAGCTTGTTCTTGCCCCAGGGCCCTTGCACTTGC
TCTCCTGTCCCCAGCTGGCTCTTCTCCTTCAGGTCTCAGCTTGTGTCACCTCCTCAGTGAAGCCCTCCCT
GACCACTACCTCCAAGCTACAATTTCAGGGCATCATGCCTTCCCAGGCCTTGCCACCAATTTTGTTTGTT
TGTTTGTTTGTTTTGAGGCAGAGCCTCACTCTGTTGCCCAGGCTGGAGTACAGTGGCATAATCTCGGCTC
ACTTCAACCTCCACCTCCCAGGTTCAAGTGATCCTCCTGCCTCAGCCTCCCAACTAGCTGGGATTACAAG
CGTGTGCCACCACACCCGGCTAATTTTTGTGTTTTAAGTAGAGATGGTGTTTCACTATGGTGGCCAGGCT
AGTCTCAAACTCCTGGACTCAGGTGATCCGCCCACCTCAGCCTCCCAAGGTGCTGGGATTATAGATGTGA
GCCACCATGCCTGGCCAATGGAATTTTTTTCCTTTTTTTTTTGTTTTGTTTTGAGACAGAGCCTCACTCT
GTGGCCCAGGCTGGAGTGTAGTGGTGCAATCTCGGCTCACTGCAACCTCTGCCTCCTGGGTTCAAGCTAT
TCTCCTGCCTTAGCCTCCCGAGTAGCTGGGACTACATGTGCGTGCCACCATGCCCGGCTAATTTTTGTGT
ATTTAGTAAAGACCGGGTTTCACCATGTTGGCCAGGCTGGTCTTGAACTTTTAACCTCAGGTGATCCACC
CGCCTCGGCCTCCCAAAGTGCTGGGATTATAGGCATGAGCCAGGAGCCACCAATCCCAGCCAACAGATAT
ATATATCAGATATATATATACTTTTTTTTTTTTTTTTGAGACTGAGTTTTGCTTTTGTTGCCCAGGCTGG
AGTGCAATGGCGTGATCTCGGCTCACCGCAACCTCCGCCTCCTGGGTTCAAGCAATTCTCCTGCCTCAGC
CTCCCGAGTAGCTGGAATTACAGACATGCACCACCACGCCCGGCTAATTTTGTATTTTTAGTGGAGATGG
GGTTTCTCCATGTTGGTCAGGCTGGTCTTAAATTCCCAACCTCAGGTGATCCGCCCATCTCGGCCTCCCA
AAGTGCTGGGATTACAGGCGTGAGCCACCGTGGTTGGCCATATTTTAAAAATATATTTCTTAAGATAAAT
CTTTGGTAAACTGTAAATTCAGTGAATCTCACATTTCACCAAGTTTCCTGCTCCCCCGAATTCACATATG
TCTAGGGCGAGGCCTGCAGGGAAAGTGCTTTGAGGTGGGATCGTGTCCTCACATCATTAGATGGGGATGA
CACTGGGAAGGACAGAGGTGACCCGAATGTCCTTATCCTGTAGGTAAAGGGAGCTATGGAGGGTATTTGA
GCCTCTTCCTCACTTACCCAGCATGGTCAGTGTTGGAGGGCCCAAGGAAGGGCTCAGAAAGATGAAGGTG
CAGGCGATGTGTAACATCTGTCCCCACGCCATCCTTCCCGGCTTTGAATCCTGGTTCTTTTGTGATACCC
CGAGCTCTTAACTTCCCCCACTGGCCTCAGTTTCCCCTCTATAAAATGAGAACCACCATGTTACCCTACC
ACCTAGGACACTCCCAGGCCTAGGTTTGAATTGCAGTATGCTGTGTGTTCTCAGGCGAATCAACTAAACT
CCCTGGGCCCCCGTTTCCTCCCTCCTGCTCCCTCGACGCCCCCTCTCCTTTTTCTGCAGCGAGATTAACC
GTCTGGATTTGGGACTGACGGTGGAGGTGTGGAATAAGGGTCTCATCTGGGACACAATGGTGGGCACTGT
GTGGATCCCACTGAGGACCATCCGCCAGTCCAATGAGGTGAGTGGCGGGTGGCCTGAGGGTGAAGAAGGA
GGAGTGCCTTGCAGAAGCCCAGAGACCAGTGGACTAAACAAGATGGGCATTTATCTCCTATCCCCTGACA
ACATCAATGTAGACACTTGGGATTGCCACGAACCTTCCAAAGTCACTAGAGCCTATACACCTTCCATCTT
GCTCTGCCTCTCTCTCCATGTGGCTTCCACCTTGTGGCCCCAAATGGCTGCTTTAGGCTCACCATTGCAT
CTTCATTCCAACCACCGAAAGGTGCTGGTGGTGGGGGAGGGGGGTGGTTCATGCACATCTCATTGGCCAG
AACTTAGACATGTGGCCAACACCTTGCTGCAAGGGATTCTGGGAAATGTAGTCCTAGGCTGGAAAATGTA
GTCCTGGGCTGTGCTTCCCTAGGCACAGCTAAAAATGTATTACCTTGGAAGGAGAAAAGGGATAATATAG
GCAATTAGCATTTTGTGTCCCAGATGGGCAGCAGCAGGAAGCTTACACCAACTCATAAGAAGGTGCTAGG
TAACAACCCTGGCCCCTCAGCCTGTGCATCATTGTCAGCAGGTAGTAGTGATGTCTATTTTTTATTTAAT
TATTTATTTTTGAGACAGGGTCTCACTCTGTTGCCCAGGCTGGAGTGCATTGGCATGATCATGGCCCACT
GCAGCCTCGACTTCCCAGGCTCAAGTGATCCTCCCACCTCAGCCTCCTGAGTAGCTGGGACTGCAGGTGC
ATGCCACCAGGCCCCCCTTATTTTTTTTGTATTTTTAATAGAGACAGGGTTTTGCCATGTTGCCCAGGCT
GGTTTCGAACTCCTGGGCTCAAGCAATCCACCCACCTCAGCCTCCCAAAGTGCTGGTATTACAGGTGCGA
GCCACCATGCCAGGCCGTGGTCATGTCTATTCATCTGTCCCCACAGCCCAATCCCTGGCCTCCTAACCTC
AAGTCTCAACCTCTCCATGCTGACCCCAGGAAGGCCTTATTAGCTGGAGCTCTGATCTGTCCTCCCCTGT
CCATCTGCCTTGGGAGGCCCTTCAGATGGCACCTGCCCACTAGCCTCATCTCCCCCCACACTCTAACCTC
TACCCCTCCCAGATTCCACCCTCTGAACCTTTGCATAGTGTGTTCCCTGAGCTTCAAGTGTCCTCCTTTC
CATCACCTTCTAAATTCCCATGTGCCCACCTCCAGGAAGCCCTCTCTGACTTCTCTCTAGATCCTTTAGC
CCTCCTTCCTCCCTCTGGGCTATGACCTCATGGGGCTGGGGGTGCCTGTGTCCAGCCCTGTTTCCAAGGG
CTATGGGCTCTCAGGAGCCAGGGTGATGATGGCTGGGCCTCTTGGGGACCCTGGCATCACCCAATATGAA
CCAGGCATAGAGGAGGGACTGGGGACTGTTTTTTGTTTGTTCGTTTTTGTCATTTTATTTTATTTTATTT
TATTTTATTTTATTTTATTTTATTTTATTTTATTTTACTGTGAGATGGAGTCTCACTCTGTCACCCAGGC
TGGAGTGCAATGGCACAATCTTGGCTCACTGCAACCTCTGCCTCCCTGGCTTAAGTGATTCTCCTGCCTC
AACCTCCTGAGTAGCTGGGATTACAGGGGCCTGCCACCACACCCAACTAATTTTTGCATTTTTAGTAGAG
ATGGGGTTTCGCCATGTTGGCCAGGCTGGTCTTGAACTCCTGACCTCAAGTGATCCGCCCACCTTGGCCT
CGCAAAGTGCTGGGATTACAGGTGTGAGTCACCGCACCTGGCCAGTGAGTGTTTGTTTTTGAATGAATGA
ATGCAGAGGGACGAAATGGTTGCAACAATCTAGGTAAGCGAGTCTAAAAGGAAGCCTTGGCTGGGCATGG
TGGCTCACACCTGTAATCCCAGCACTTTGGGAGGCTGAGGCAGGTGGATTGCCTGAGCTCAGCAGACCAG
CCTGGGCAACATGGTGAAACCCCGTCTCTACTCAAAAATACAAAAAATTAGCCAGGAGTGCTGGCACGTA
CCTGTAATCCCAGCAGCTCAGGAGGCTGAGTTAGGAGAATCACTTGAGCCTGGGAAGCGGAGGTTGCAGT
GAGCTGAGATCACGCCACTGCACTCCAGCCTGGGTGATAGAGAGACTCTGTCTCAAAAAAATTAAATTAG
GCCGGGCATGGTGGCTCATGCCTGTAATCCCAGCACTTTGGGAGGCCGAGATGGGTGGATCACGAGGTCA
GGAGATTGAGACCATCCTGGCTAACACGGTGAAACCCCGTCTCTACTAAATATACAAAAAATCAGCCAGG
CGTGGTGGCGGGTGCCTGTAGTCCCAGCTACTCGGGAAGCTGAGGCAGGAGAATGGCGTGAACCCAGGAG
GCAGAGCTTGCAGTGAGCCGAGATCGCGCCACTGCACTCCAGCCTGGGCGACAGAGCGAGACTCTGTCTC
AAAAAAAAAAAAAAAAGAAAAGAAAAGAAAAAAGAGAGAGAAAGGAAGGAAGGAAAAGAAAGGAATAAAG
GAAGGAAGGGAGGAAGGGAAAGAAAGAAAGGAAGGAAGCAGGGAGGGAGGGAGGGAAGGAAGGAAGGGAA
GGAAGGAAGGACGGAAGGAAAGATAGAGAGGGGACATGACTTTGGTTTTGGGGTAGGGGTGAGGGGGATG
TCTCATGCGAGAGTAGGGGCCCTGGGGACTAGTGACCAGCAGATTCCTGACCCACACACCTCCTCCCCCA
ACCCCCAGGAGGGCCCTGGAGAGTGGCTGACGCTGGACTCCCAGGTCATCATGGCAGACAGTGAGATCTG
TGGCACCAAGGACCCCACCTTCCACCGCATCCTCCTGGACACGCGCTTTGAGCTACCCTTAGGTGAGTAC
AGGGGCCCGGGACCTTCACCCAGCCCCAGCCCCAGTTGTGGACTCACTCAGCTATTCCAGGAGAGTGAAC
AGGCCTGGGTAGGTCTAGCTGACCCTTCAGGCTGAGAATGAGGGTCCTTCGCCCCGGAGGAGAGAGAGTT
TCTTGGGTTGCAGAGAACAGGGATGTGAACCTGCTGGGTGCGGGCCATGACCTGGGCCCCGTAGCTTCCC
AGTGAGCTGCAAGTGCTTCACGACCGAGGGCGTTCAGGGTTGAGCTATCCTGGCTGTTCCTTGGTGATGC
CTGAGACTGCAACAGTGAACTGGGGATAGGAGGAGAGACAGACAGAGACAGAGAGACAAAGAGAGACAGA
GAGAGACATGTAGACAGAGGCTGAGAACAGATAGTCCCCTCTTCCTTTCCTGGAAGCACCTTGAGTCTCT
CCACCTCATTCTTCTAAGAACCCATGGCTGGCCAGGCATAGCTTCTCATGTCTGTAATCCCAGCACTTTG
GGAGGCCAAGGGGGGAGGATCATTTGAGGCCAGGAGTTTGAGACCAGCCTGGGCCATCTAGTGAGATCCC
ATCTCTATGAAAAAGTTAAAAATTCACCAGGCATGGTGCTGGGTTTCTGTAGTCCCAACTATTTGGGAGG
CTGAGGCAAGAGGATGGCTCAAGCCCAGGAGTTTGAGGCTTCAGTGAGTTGTGATTGTGCCACTGCACTC
CAGCCTGGGTGACAGAGCGAGACCCTGTCTCTAAAAAAATAAAAATAAAAACAATGGGCTGGGCGTGGTG
GCTCATGCCTATAATCCCAGCACTTTGAGAGGCCGAGGCAGATGGATCACCTGATGTCAGGAGTTTGAGA
GCAGCCTGGCCAACATGGAGAAACCCTGTCTCTACTCAAAATACAAAAAAATTAACTGGGCATGGTGGTG
CACACCTGTAATCCCAGCTACTCGGGAGGCTGAGGCAGGAGAATCGCTTGAACCCAGGAGGTGGAGGTTG
CAGTGAACTGAGATTGTACCACTTCACTCCAGCCTGGGCAACAGAGTGAGACTCCGTCTCAAAAAACAAT
TTAAAAATTAAAAAATTAAATAAAAACAATGGCTTCCCCACCCTCGTAAAGGCTAGGAACCATCTCCTGT
CATCCTCCAACTATTCTCCATTCAGCTCTGGGAAGAGGGGCGTCAGAGGCACCACGGTACCTCCCCTTCA
TCTCCATCCACCCTCCTGAGACCGCAGACCAACCCACCCCTACTTCTGAGGCTTTGCCAAAGACCCCAGA
AGCCAGGGCCCGGGTGGAGCTCAGTAGGCCTGGAGGATGGAGGCTGAGCGGCAGGGAGGGTCTTCCAGGC
TCACAGACAGGGCTCTGCTTGCAGACATTCCTGAAGAGGAGGCTCGCTACTGGGCCAAGAAGCTGGAGCA
GCTCAATGCTATGCGGGACCAGGATGAAGTGAGTGTTGGGGTGGGAGGGGACTGTTTCTTCCTTGTTTCT
GTCTCTGTCTCCCCATGATTTTCACTTACAGATGCTGTTTCTCTCTAAGCTTGTCTTTCCCTGCATCTCT
GTTTCTAACTCTTTGTAACCTGACTGCATCCATGACCCATGTGGTCCAGTCTCTAGACAATGATGGGGTC
AAGAAACACACAGAGGCCAGGCGCAGTGGCTCACGCCTGTAATCCCAGCACTTTGAGAGGCGAAGGTGGG
CAGATCACCTGAGGTCAGGAGTTCAAGACCAGCCTGGTCAACACAGTGAAACCCCGTCTCTACTAAAAAA
AAAAAAAAAAAAAAAAAATTAGCCAGGCTTAGTGGCTCACACCTGTAATCCCAGCAATTTGGGAGGCTGT
GGCGGGCAGATTATTTGAGGCCAGGAGTTCGAGACCAGCCTGGCCAACACGGTGAAACCCCGTCTCTACT
AAAAGTACAAAAAATTAGCTAGGCATGGTGGCGGGCGCCTGTCATTCCAGCTCCTTGGGAGGCTGAGGTG
GGAGAATCACTTGAATCTGGGAGGCAGAAGTTGCAGTGAGCCGAGATCACGCTGCTGCACTCCAGCCTGG
GCAACAGAGCAAGGCTCTGTCTCAAAATAATAATATTGATAATAATAATAAAATAAAAAATTTTACTTTT
CATTTGGCGGATACATGTGCAGGTTTGTTACACGAATATATTGCATGATGCTAAGGTCTGGAATTCCATG
GCATTACTCAAATTGTAAACACAGTACCTAACGGGTAGTTTTTTCACTCTTGTCCTGCTCCCTCCTTCCT
CCCTTCTGGAGTCCCCAATGTCTACTGTTCCCATCTTTTTTTTTTTTTCTTTTTTGAGACGGAGTCTCGC
TGTGTCACCAGGCTGGAGTGCAGTGGCACGATCTTGGCTCACTGCAACCTCCACCTCCCGGGTTCAAGTG
ATTCTCCTGCCTCGGCCTCCCGGGTAACTGGGACTACAGGCATGCGCCACCATACCTGGCTAATTTTTGT
ATTTTTAGTAGAGACGGGGTTTCACCGTTTAGCTAGGATGGTCTCGATCTCTTGACCTCGTGATCTGCCC
GCTTCGGCCTCCCAAAGTGCTGGGATTACAGGTGTGAGTCACTGCACCTGGCCCCTAGTCTTTTTTCTAT
CTCTGTTTCTTACTTTTTCTGTCTCTGTCTCTGTCTCTGTCTTTCTCTCTCTCTCTCTCTCTCTCTCTCT
CTCGTGTGTGTGTGTGTCTTTCTCTCCTTTTCTCTCTTACTCCCTGTATCCTTCTCATAGAGAGGGAGAG
AATTTGGGAGGTAGAGGGAAGTCGGGATGGGACAGAACAGAGGACTATCCTGGTCCCCTGGCCTTTGGCC
CCTCTGAGAGAAGCCCTTTCTCCTTCCTCCACCCCTTCAGTATTCGTTCCAAGATGAGCAAGACAAGCCT
CTGCCTGTCCCCAGCAACCAGTGCTGTAAGTACCTCTTGATTCTTCCTTGGGTATCAGCTGAAATCCTCA
TCCCTCCCCTACCCCATGACTGTCCTGTCTAAAGGCTCCTCCAGTCCGGAGATACAAGTCCTGAGAGTGC
AGTGTCCACTTACAGTGTATTAGAAATCAGCGTTCTGAGGCTGGGCATGGTGGCTCACCCCTGTAATCCC
AGCACTTTGGGAAGCCGAGGTCAGGAGTTCGAGACCAGCCTGGCCAACAGGGCAAAACCCCATCTCTACT
ATAAATACAAAAAAAATGAACAGGCATGGTGGTGGGTGCCTGTAATCCCAGCTACTCAGAAGGCTGAGGC
AGGAGAATTGCTTGAACCCAGGAGGCAGAATCTGCAGTGGGCCAAGATCATGCCACTGTACTCCAGCCTG
GGTGACAGAGCAAGACTCTGACTCAAAAAAAGAAGGAAGGAAAGAAAGAGAAAGAAAGAGAGAAAGAGAG
AGAGAGAAAGAAAGAAAGGAAGGAAGGAGAGGAGAGGAAAGAAAGAAAGAAAAAGAAAGAAAAGAAAAGA
GAAAAGAAAAGAAAGGAAGGGAAGGAAAGAAAGGAAAGGAAGGAAAGGAAAGAGAAGAGTTCTGAGACTT
GGGCTTGATGAAAGACAAGAAGGAAGAAAGGAAGGAAGGAAGGAGAGTTCTGAGACTTGGGCTTGATTCC
CACCTTGCTATGTGGTCATGGGCAGGCCAGTGTCCCTCTTCGATATTTAGGGGCCCTCTCTTTGAAATAG
CGCATCTGACAGCCCCCTCACTCTGAGGGCTGTTGGAAGGATCTGGGACATCTAGAGACATGAAAAACAC
TTCGTAGATGCTTCACTTCCATTCATCAGATCCTGGATGAGGGCGTATAACTCATTCCACAAACTATTCA
TGGGGCCAGCACAAACCCCGAGCAGCCCACGTCAGATGGTGCAGACAGACACAGGCAGAAGATCCTGCCA
GCACATGTACAATGGGCGCACGTACAACAGACGAAGGCTGTTTCACTAACCCATTTTCTTAGATGGAGAA
ACTGAGGCCCAAGAGGAGATGTGATTTACCCAAGGCCACACACCCGGCAACTGGCAGAGCCAGGATTTGA
ACCCAGGTCTGAGGGACTCCAACTTTGCTTCCCTTTCCACTGCACCTTGTCTGTTAATGATGATGATGAT
CACGTTCCTCTTCCTATTTCCATCAAGGCTGCAGTTGTCAAGATTTTTTATCTTCCTGGGCCTTGTGGAC
ACTCAGATGCATCCAGACCAGGTCTTTTCATGGATGGAGGAGCTGTCTCCCTCCTTAAAATGACTTCCCA
TTGCTCTGTGGTGGAGGGGAAGTAGCAAATCATATAGCCTCAGGGCAGAGCTCCCACCTGCTGACACCAA
AACCCCACTGCACTCTTTAGCCTCCTTCTCCTTTCCCATTTACTTTCTTTTCTTCTTCTCTTTTTTTTTT
TCTTGGAGATAGAGTCTCCCTCTGTCACCCAGACTGGACTGCAATAGTACTATCTTGGCTCACTGCAGCC
TCTGCCTCCCGGATTTGAGTGATTCTTGTGCCTCAGCCTCTTGAGCAGTGGGGATTACAGGCACATGCCA
CCACGCCCAGCTAATTTTTGTATTTTTAGTAGAGATGGGGTTTTGCCATGTTGGCCAGGCTGGTCTCAAA
TTTTTTACCTCATGTGATCTGCCCACCTCAGCTTCCCAAAGTGCTGGGATTACAGGTGTGAGCCACCATG
CCTGGCACCTTTTCCATTTGCTTTTCCATCTTCCTTTTCTCCCCTCTCACAGCTCTCCCTGTATCTGTGT
CTCCCTCACCATGTGTCCAGGAGGGTGGTAATGGAACCGGGGTTGAGGAGGGCTTGGGATACTCCAGCTG
GGGTACCCTGTTTCCACACTAACTTGGGGAGTGATATTGAGAAAGTTCCTTGGCTTCCCTGGCCAGGGAG
GGGTCATCACTGCCTGGAAGGAATTACAAACCCCCGCTTGGTGAATGGGGTCATTGAGAGGTTGCAATGG
GGGCGGGGCCAGGCAGAGTGGAGGCAGGAAGGCAATGGCAGAGAGCCCCTGGGCAGATGGACTAGAGCTA
GGGGCAGGTTCATTTTGGGAAGTGGAGACCACCACTCAGTATTTACCCACAAGTCCTCCCTTGGAGTATT
TTGTCACCCTATTTCTCAGAGAAAGCAGCCAATGGTCAGAGGAGCCAGGATTCTGGTCCAGGACATTTTT
CTGAAGTGCCAGGTACTGCACGTGATAGGCAAAGGTGGTTCTCGCCCTGGCCCTGATGCGCAGAGGCTCT
GTGACTTTGGGCTGCTCACTCCATCCCTCTGAGCCCCATAGTTTCACCCATAAAATGAGTAGAATGATAA
AATCCACCCAACAGGGGTGTTTCAAACCTTAGGGTTTCCAATTTAGTGTGTCTTTCCTTATCCAAGAGCT
GGGAGAGGAAAAGGGTGGAGACTGGGAGACTGCTAACCCTAGACATCATAGTCAGAAAACCCTGGGCCCA
AATACCCACTTTGTTACTTCCTGACTGAGCCTTTCCTCTCATTGAAAACTGAGCCAGGCACAGTGGCTCA
TGCCTGTAAATCCCAGCTACTCGGCCTCACTTGAGACCACGTGTTTGAGACCAGCCTGACCAACATAGCA
AGATCCATCTCCACACAAAAAAACAAAAAGAAAACCACCAAGAACCAAAACATTAGCTGAACATGGTGGT
GTGTGCCTGTAATCTCAGTTACTCAGGAGGCTGAGGTGGGAGGATCACTTGAGCTTGGAAATTGGAGGCT
GCAGTAAGCCGTGATTGCACCACTCTACTCCAGGCTGGATGACAGAGTGAGACCGTGTTTCAAAAAAAAA
ATTAAAGAAAAGAAAAAAATAAACAAACAGGGATATCCCACCTTCCTCCCAAGGTGGAGAGCAGAGACAC
AGCAAGTGTCAGCAATGAAGGTGATACTCAGGGAAGGTACTCAGGACAAGAGGGACTTTTGGGGTGGGGG
GAGCAGTTGGGGAGAGTGAGGACAGCAGGGGAGCCCCCTTCCTACACCCACCCCTCTGTCTGCTCCCTTT
TATTATTTCTGCCCCTTCTCTTTGTAGGCAACTGGAATTATTTTGGCTGGGGTGAGCAGCACAGTAAGTA
CTCAGCCTGATTGATGTTCTGGGAGTTACATGGGAAGGTGGGCACCTTGGTGATCCCAAGGCCCCTCCCC
ACTACCACACAGGGAAGCAAGGAGCCTGTGACGAGCACGTGGGGGCCAGAGGGCTCAGCAAGACCCCTTT
CAAGCTTTCCTGTACCCTGTTCATTAGTGCCTGTCTTGGGAGGCCTGCCATCTGCTGGGGAGTGGGCACC
GTCATTGGGGTGGGGTGGGGGGGGTTAGCCTGGAGGAGTGGACATTTAGACCAACAACCTGCTTGTCTCC
AGCTGGGCACTGTGACCCCCGCACCCCACTCCGCCGTATAACCTCACCTCCTCTCCTGTAGCCCCTGCCT
TTTGGTCTTCCTATCTTCCTTCTCCACTTGGAAATGTGTCCAGTGTCACTCAGAGGGTAGTGGGTGGGCG
GGGTCTCTAACAGAACGGCAGGCCAGGGACCTCAGGGAGATGCACACAGGGACCTCAGCACCCCCCAGGT
CCACCCACACTCACACCTCACCTTCCACCTTCATTTGCTCTCCCAGGGAATAAGGCAAGCCACCCCCTCC
CTAAATTAAATGTCCATTTTCCTTTTCCTTTTTTTTTTTTTTTTTGAGACGGAGTCTCACTCTATCACCC
AGGCTGGAGGGCAGTGGCACGATCTCAGCTCACCGCAAGCTCTGCCTCCAGGGTTCACGCCATTCTCCTG
CCTCAGCCTCCCGAGTAGCTGGGACTAAAGGCGTCCGCCAACACGCCCGGCTAATTTTTTGTATTTTTAG
TAGAGACGGGGTTTCACCGTGTTAGCCAGGAGGGTCTCGATCTCCTGACCTCGTGATCCACCTGCCTTGG
CCTCCCAAAGTGCTGGGATTACAGGCATGAGCCATGGCACCTGGCCCTAATTAAATGTCCGTTTTTCTAC
CTAACAGCGGTTCCCAACCTTTTTGACACAAGGGACCAGTTTCATGGAAGACAATTTTTTTATGGACTGG
GGTTGGGGGATGGTTTCTGGATGATTCAAGTTCACTGCATTTGTTGTGCACTTTATTTTTATTATTATTA
TTATTACATTATAGTATGTAATGAAATAATGATACAACTCATCATCATGTAGAAGCAGTGGGAGCCCTGA
ACTTGTTTTCCTGCAATTAGACAGTCCCATCTTAGGGTGATGGGGGACAGTGACAGATCATCAGGCATTA
GATTCTCATAAGGAGCACACAACCTAGATCCCGCACATGCACAGTTCACAGTAGGGTTCACGCTCCCGTG
AGAATCTTTTTTTTTTTTTGAAACAGAGTCTTGCTCTGTTGCCCAGGCTGGATGGAGTACAGTGGCGCAA
TCTCGCCTCACTGCAACCTCTGCCTCCGAGTTCAAGTGATTCTCGTACCTGAGCCTCCCAAGTAGCTGGG
ATTACAGGCGCGTGCCACCATGCCCAGCTAATTTTTTGTATTTTTAGTAGAGATGGGGTTTCACCATGTT
GGCCAGGCTGGTTGTGAACTCCTGACTTCAGGTGATCCACCTGCCTTGGCCTCCCAAAGGGCTGGGATTA
CAAGTTTGAGCCACTGCGCCCGGCCTTCTATGAGAATCTAATGCTGCCGCTGATCTGACAGGAGGTGGAA
CTCAGGCGGTAATGTGAGTGATGGGGAGCAACTGTAAATACAGATGAAACTTCACTCGCTCACCCAACCG
CTGCCCACCTCCTGCTGTGCGCCCCAGCTCCTAACAGGCCACAGACCAGATCCATGCCCTAAAGAGTTGC
TCCCTTTCTCCCATACCCCGCCTCTTCTAGAGAAATCCAGTCCTCTATTCTTTCTTTCCTTCTTTCTTTT
TTCTTTTTTTTGAGATGGAGTCTTGCTCTGTCACCCAGACTGGAGTATAGTGGTGCGATCACTGCTCACT
GCAACCTCCACCTCCCGGGTTCAAGCAGTTCTCTTGCCTCAGCCTCCCGAGTAGTTGGGATTACAGGTGC
ACGCCACTATGCCTGGTTAATTTTTGTATTTTTAGTAGAGATGGGGTTTCACCATGTTGGCCAGGCTGGT
CTTGAACTCCTGACCTCAAGCAATCCTCCCGCCTTGGCCTCCCAAAGTGCTGGGATTATAGGTGTGAGCT
ACCATGCCTGGCCAGTCCTCTATTTTGAGGATAAATCTGCCTCCTTCTCTCTCCTTCAACTGGGGAGTCC
TTCACTCCTTCCTCATATCCTGGTGGACAAACCTAACTCCAGGACCAGTGAAACTGTTGACAAACACTTT
GGTCCACCACAATTTATCAAAAACAAGTTGGTTAAAAATCAACTTAGGCCAGCCACACTGGCTCACACCT
GTAATTCCAACACTTTGGGAGGCTGAGGCGGGAGGGACGCTTGAGCTCCAAAGTCTGAAATCAGCCTGGG
CAACAGAGCAAGACTCCATCTCTACAAAAAAAAAAAAAAAGGGCCGGGTGTGGTGGCACGCCCCTGTAGT
CCCAGCTATTTGGGAGGCTGAGGTGGGAGGGTTGTTTGAGCCCAGGAGGTTGGGACTGCAGTGAGCCATG
ATTGTGCTACTAGACTTTAGCCTAGGCAACAGTGTGAGACCCTGTCTCTAAAAAATAAATAGATTAAAAT
AAATAAATAAAATCAATTTTGCCAAAATGAATGCTTGGCAGACAGGGCAAGCTCGATAACAACCAACTTG
CCACAAACGAACAGTACGGTCAAAAGCAAAGATAGACCAGGCGTGGCAGCTCACACCATAATCCCAGCAC
TTTGGGAGGCCGAGGTAGGCAGATCACTTGAGGTCAGGAGTTCGAGACCAGCCTGGCCAACGTGACGAAA
CCCCATCTCTACTAAAAATACAAAAATTAGCCAGGCATGGTGGTGCATGCCTTTAATCCCAGCTACTCGG
GAGGCTGAGGCAGGAGAATCATTTGAACCTGGGAGGTGGAGGCTGCAGTGAGCCGAGATCGTACCATTGC
ACTCCAGCCTGGGTGACAGAGCGAGACTCTGTCTCAAAAAAAAAAAAAACCCAAAAACCAACAAACAAAC
AAAACAAAACACCAATGCAGCTAAAGCTGATAGATAATTGGGTCAAAAAGGAAAGACAATTGAACCGAAA
ACAGACCGTTGGGTTAAAACAAAAGTGGTTGAAAATAGCCAGAAGCAGGCAATTTAGTCAAGAGCTACTT
AGTAGAAAGCAGAGCCTGGGCAAAAGCAAAAAGAATCCAACTCAAAGTAATTTACCCTTCATAAGCAGAT
AATTTGTCAGGAAAAAAAAAAACTGGGTAAACATGTGCACAGCCTCATGCAATTTGGCTGGAAACATCTC
AGCCCGGTCAAGGACAATTGGATAGAAAGAATGCTGGCCGGCCGAGGTGGCTCACGGCTAATCCCAGCAC
TGTGGGAAGCTGAGGCGGGAGGATCGCTTGAGCCCAGGAGTTCAAGATCAGCCTGGGCAATATAGGGAGA
TCCCATCTCTACAAAATAATAAATAAATAAAATTAGCTGGGTGTGGTGGTGTGTGCCTGTGGTCCCAGCT
ACTGTGGAGGCTGAGATGCGAGGATCTCTTGAGCCCAGGAGGTCGAGGTTTCAGTGAGTTTTGATCGCGC
CACTGCGCTCCAGCCCAAGTGAGAGAGACCCTGTCTAAATGAATGGATGAATACAGCAAAAGTCTGAAGG
TTTCACTCATTGCTCTGAGATTTTGGGGTCTGGGGGTTTTTTCATTTTTTTACATCCTCTTCCCCTTACC
CGTACTTCTCATGTTTTCTTCATGGACTACTTTTTCTTTTTTTCTGGATACAGGGTCTCATTCTGACACC
CAGACTGCAGTGCAGAGGCGTAATCATAGCTCACTGCTACCTCGACCTCCTGGGCTCCAGCAATGCTCCT
GCCATACCCTCCCGAATAGCTGGGACTACAGGCGCCCACCACCACGCCCAGCTAATTTTTAAATTTTTTT
AAAGAGATGGGGCCTTACTATGTTGCCCAGGCTGATCTTGAACTCCTGGGCTCAAGTGATCCTCCTGCCT
TGGCCTCCCAAAGTGCTGGGATTACAGGCATGAGCCACTTTGCCTGGCCCATGGACTAGGTTTTACTTTA
CTTTTATTTTATTTTATTTTTTGGAGACAGGGTCTTGTGTCACCCAGCCCCTGGAGTGCAGAGGTGTGAT
CTTGGCTCACAGCAATCTCCACCTCCTGGGTTCCAGAGATTCTCCTGCCTCAGCCTCCCAAGGAGCTGGG
ATTACAGGCACATGCCACCACACCTGGCTAATTTTTGTATTTTTAGTAGAGACAGGGTCTTGGTATGTTG
CCCAGGCTGGTCTTGAACTCCTGGCCTCAAGTAATCTGCCCGCCTCCGCCTCCCAAAGTGCTGGGATTAC
AGGTGTGAGCCACTGTGCCCGGCCTTTGCTTTACTTTTAAAATAAAGATAACTTTATCATTCTTTTTAGT
TAAAAAATTAAAAAGATTGCATTATTGAAATCTTTTTTTTCAGAAGGTACAGAAAAGAATAAAGAAGAAA
ATGTACAATCCCCAATTATGCCACCACCCAGACATGTTGCTTTTAGAATTGGAATTTTTTTTTTTAGGAT
TTAGAAAACAAACTGTTCTTTTTTCTAAGGAAACCAAACTAAACCAAAATATCTTTTAGTTCTTATTAAA
CATGAAGTTTGGCAACTCTACCACATTCCATTTTAGCAATAGATTCCAAGTGTGTGGTGGCTTTGCCAAG
CTAAGAATCTAATCCTTTTTGAAAAAGACAGAACCATGTCTCAGCATCTTCTTGGTTCTTGCATATTTGT
TAAGTTCAGGTCTGGGGTCGTCACCCTCAGATATCAGGAAATCTCTAAGGGTTAAAGAGCGTTCCGTCCA
CTTTGAACTCCTGATCTTCTTCCAACTTCCCCCTGACTGTCTGTGTGACTCCTGTTATGGGAAACTAACT
GCTCAGTGGAAAAAAATCCCATGTCATTTTCAGAGATCCTGACCTTCAGCAAAGTTGTTCTTCACATTTG
AGCTGAAGGATGCCCTCTACTCTGTCCCTGTTTTCTTCACATTGTAGGTCCTACCCTAGCCCCTAGACTT
TAGGTCCAATTTGACCCACATAAAGCAAAGCAGGGTTTTCACCTCCCTTGTTCTGAAGCCTATATCTCTA
TTAACATGACCCACAATTCTTTATCCATACTAGTAAAAAATTCTCTTCTTCGGTAGCGGCACCGTAAGTC
TGGGAAATTGTATTGGCTCCCATCATGTGCACTCTCTCTTCCTCACCCTCTTCCCATACCGCCTCTCTCT
TCCAGACGATGACCCCGACAGTGCAGTGGATGATCGTGACAGTGACTACCGCAGTGAAACGAGCAACAGC
ATCCCGCCGCCCTATTATACTACGTCACAACCCAACGCCTCAGTCCACCAATATTCTGTTCGCCCACCAC
CCCTGGGCTCCCGGGAGTCCTACAGTGACTCCATGCACAGTTACGAGGAGTTCTCTGAGCCACAAGCCCT
CAGGTAGGTGCTGCGGAGTGCAGCATGCATGTGGGTCCTGCTGTGTCTCCTGGAGTGGAGAGAGGGGTAC
GGGGCTGGACAGAGCTGGAGGTGGAGTTCCAGAAGGGCAGAATTCACCCATCCAGGAGGGCAGAATTCAC
ATTTTCTTGTGGGAATTAACCATTTTTTTCTTTTTTTTTTTCTTTTCTTTTTTTTTTAGACAGAGTCTTG
CTCTGTCACCCAGGCTGGAGTGCAGTGGCACCATTTCGGCTCACTGCAACCTCCACCACCCAGACTCAAG
CGATTCTCCTGCCTCAGCCTCCCCAGTAGCTGGGACTACAGGCACAGGCCGCCACACCAAGCTAATTTAT
TTTTATTTTTTGTGGAGATGGGGGTCTTGCTCCATTGCCTGGGCTAGTCTTGAACCCCTTTTTATTCCTA
TAGCATCCTCGAGGCTGGCCAGGGAAGAGGGTCTCAGGCGCTAAGATTGGAGGAGGTATCTAATCGGGAA
ATGATAAGGTTCGTTGAATGACTAAGTGAATGGTTAGAAGAGGGCTGAGTGGTGGCCCCTATGTCCCCCT
GCCTGTATTTAGACAGAGACAGATATTGCTGTTGACGTTGAGCACGATCCTGGGTTCACATAAGCAGCAC
ATAACAGAGGGCCTGACCTGGCCAGACAGTGGGGGGCATTTGCACAAGTAAGTGATGTTTCAGTGGGTAC
CAGAGGGTGGGTAGGAGTTTGCTAGATAAAGCAGACAAGGAAGAGACCACGGCAAGTGCACAGGTTTATG
GTGTTGGAGAGACAACGAGGTGGCCAGCGTGGCCGGAATGGAGGAGGCGGGGAGAAGTGGAGGGAGGTAT
GCAGGGGGAGTTTGGAGCGTATGGGCCACACAGGGCTAGCACAGATTTTATTCCAAGGCACTAGGGAGCC
ATGGTAGGATTTAGAGCAGGGGAGGGAGGTGATTGGATTTACAATTCCCTGTGGCTTCCAAGGGAAGCAG
GAACTGTGGAGACAGCATCTGGTAGAGACAGCAGAGGCTGGCCTGGGTTGGGGCCCGGGGTACGGGGAAG
TGGATGCATTTCCCATACTTTTTCCAGGTGGAGGACAGGAGACCTGCTGCGGGGCTGTGGTGATGGCGCT
GACCCTGAGTTCCCAGCACAGTCAATTGCCTGTTCCCCACAGACCGCAAATGCCACAAAAGTGGGAATTT
TTTTTTTTTTTTTTTTTTTTTTGAGACGGAATTTCGCTCTTGTTGCCCAGGCTGGAGTGCAATGGCATGA
TCCCAGCTCACTGCAACCTCCACCTCCCGGGTTCAAGTGATTCTTCTACCTCAGCCTCCCGAGTAGCTGG
GATTACAGGCATACGCCACCATGCGCAGCTAATTTTTGTATTTTTGGTAGAGACAGGGTTTCACCATGTT
GGCCAGGCTGGTCTTGAACTCCTGACCTCAGGTGATCTGCCCACCTCAGCCTCCCAAAGTGCTGGGATTA
CAGGCGTGAGCCACCGCACCTGGCCAAAAGCGGGGATTTTTGTCTTTTCTGATCATGGCTGTGTTTTCCA
GCCCCTAGAGCAGGACCTGGCACATCGTAGGTGCTCAGTCGGTGATGAGTCAATGCCTGTGTGACTGGGG
CTCAGGTGGGCAGTGACTCGGTACCCCAGTCCCTGAACCCTGTTCCATCCCCACAGCCCCACGGGTAGCA
GCCGCTATGCCTCTTCCGGGGAGCTGAGCCAGGGAAGCTCTCAGCTGAGCGAGGACTTCGACCCTGACGA
GCACAGCCTGCAGGGCTCCGACATGGAGGATGAGCGGGACCGGGACTCCTACCACTCCTGCCACAGCTCG
GTCAGCTACCACAAAGACTCGCCTCGCTGGGACCAGGATGAGGAAGAGCTGGAGGAGGACCTGGAGGACT
TCCTGGAGGAGGAGGAGCTGCCTGAAGATGAGGAGGAGCTGGAGGAGGAGGAGGAGGAGGTGCCTGACGA
TTTGGGCAGCTATGCCCAGCGTGAAGACGTAGCTGTGGCTGAGCCCAAAGACTTCAAACGCATCAGCCTC
CCGCCAGCTGCCCCAGGGAAGGAGGACAAGGCCCCAGTGGCACCCACCGAGGCCCCCGACATGGCCAAGG
TGGCCCCCAAGCCAGCCACGCCCGACAAGGTGCCTGCAGCTGAGCAGATCCCTGAGGCTGAGCCACCCAA
GGACGAGGAGAGGTAACAGGACGGGGCCAAGGGGCCAGAGGGGCTGCCACAAGGTCACAGGGTCAGCAGG
GCTATGATGGGGTCAGCGTGGACCTGGGAAGTTGCTGGGGCTGAAGTTGTCACAGGCTCTTGGGTTTCTT
AAAGGGATCAGAGGTGACAGGGCAGAGTAGGGAGGTCAAAGAGGGTTCAAGGGGCCGTGAAGGAGACTTA
GAGTTTGTGATCTTAGTGGTTGCTGGGGTCGGAGAGGTCAAGATCCTGGGCAAATGGGGGGCCAGAGGGG
CTGGGCTGGTGAAAAGGAAGCCGAGGAACAGTGAAGTGTCACAGAAGCATTGGGTGTGTGGGAAGGGGTG
AGGGGCACTGAGGTGGGCCAGGGGACTCACGTGGGTCCTAATGTCTCTGAGGGGTCAGAGATACCATGGG
GAGCTATGCATCACTTGCACAGCTGGAGGGTCAAGGGAAGTTCAGGGGTCACGGAGAGCCAGCCCAGAGC
CTCATAGCGTTCCCTGCCTCAGTTTCAGGCCGAGAGAGGATGAGGAAGGCCAGGAGGGGCAGGACTCCAT
GTCCAGGGCCAAGGCCAACTGGCTGCGTGCCTTCAACAAGGTGCGGATGCAGCTGCAGGAGGTGAGTGAC
ACGCCCAGCCATGCCCCTGCCACACTTAGCCAGGCCACGCCCAATGGCAGGCCCACACATGTTTTGGCCA
CACCCACACCTATATTGGCCACGCCCAACACCCCACCCGTACCCAAGCCTAGCCTGGGCATGCCCAGTGC
AGTGCCCACACTGTGCCCATGCCCCAGCCACTCGAGCCCTTCCCTGGCCCCACCCTCATTCAGCCTGGCC
TCACCCAATGTCACCGATGCCCCACCCACTCTGACCCTTCCTTAGCCACATACACACACAGCCCAGGGTA
TCCCCATGGCCCGTCCACACCAGCCTGGCTATGCTAACTCCCAAACCATGCACCAAGTCTGGCCCCACAC
ATCCCCAGCCCAGCCTCCTTCCCAATGTCAGCCTGGGGTTTCTAGATCTAGAGGGAGGAGAGAAAACTAG
GGCTGAAACTGGGCTGAAGTCATGGTTAGGGATCAAGGTCAGGGTTAGTTCAGCCATCAAAGATCAGAGG
TCAGGGTTAGAATTGGTGTTAGGCAAAGGGGTCAGTGCTTAGGGCCAAATTGGAGGTCATTGTCAGGGTT
AGGGAAAGGTCTGGGGCCGGCTCAGTAGGGGAGTAGGAATCAAGGGAAGCAATCAGCATCAGGGGCAAGC
TGACTGTTTAGATGGGCAGCAGGGTTGAGGCCATTCGTTTATTCCTTCATTCAGCTCACCCTGGGCTGGG
GACAGCTGTGTTGCTCTTCAGAGTTACCCTCCAAACTGCAGTGGGATTGGCAGGAGGAGATTCTAAACAG
ACTCCCAAAGGTTTAGACCATCAAATATCAGAGCCACAAAATTACCAAACGAAATTTCTTAACAGTTTTA
AAATAGAAATCTATTTTTTGACTCAGCAGTACATACATATGGTTCTAAATGAGACAGGTGGAAGAGGACA
CAGTGAAAAGGCTGTTGGATAACTTCCACTAGGTCAAGAAATTAAATTGAAAAAATAGCAGCTACTATTG
GAACTATGTGATATACAAGAAAAACAAACTATGGAACTAGTAATATGACAAGAAAAAGACACATGGAAGG
CTCTCTGGGTTTCATAGGAAAAGCATAAAACTAATCCAAGGCAATCAGGAAAACTCCCTGGAGGAGATGA
CACTTAAGGAGAAGTGATGGCCGGGCGTGGTTGCTCAAGCCTGTAATCCCAGCACTTTGGGAGGCCGAGG
TGGGTGGATCACGAGGTCAGGAGATCGAGACCATCCTGGCTAACATGGTGAAACCCCGTCTCTAGTGAAA
GTACAAAAAAATTAGCCGGCTGTGGTTGCAGGCGCCTGTAGTCCCAGCTACTCGGGAGGCTGAGGCAGGA
GAATGGCGTGAACCCGGGAAGCGGAGCTGGCAGTGAGCCGAGATCGCGCCACTGCACACCAGCCTGGGTG
ACAGAGCGAGACTCCATTTCAAAAAAAAAAAAAAAAAAAAAAAGAGAAGTGATATCCAAAATATTTATTT
CAACAATATTTCAGGAGCTCATGCCTGTAATCCCAGCAATTTGGGAGGTCGAGGCGGGAGGCTCACTTGC
ACTCAGGAGTTTGAGACCAGCCTGGGCAACGTAGTGAGACCTCATCTCTAAAAGAAAAAAATAAAAAATA
TTTCAGAGCCGGATGCAGTGGCTCACGCCTGTAATCCCAGCACTTTGGGAGGCAGAGGTGGGTGGATCAC
CTGAGGTCAGGAGTTCGAGACCAGCCTGGCCAACATAGTGAAACCCCACCTCTACTAAAAATACAAAAAA
TTAATTGGGTGTGGTGGTGGGCACCTGTAATCCCAGCTACTAGGAAGGCTGAGGCAGGAGAATCGCTTGA
ACCCAGGAGGTGGAGGTTGCAGTGAGCTGAGATTGCGCCACTGCACTCCAGCCTGGGCAACAAAAGGGAA
ACTCCGTCTCAAACAAACAAACAAACAAAATATATATATATATATTTATTTCAGGAGCTGAGTGTGGTGG
CTTGTGTGTGTGGTCCCAGCTACTCAGGAGGCTGAAGCAGGAAGATCACATGAGGCCAGGAGTTCAAGAC
CAGTCTGGGCAATATAGCAAGACCCTGTCTTTAAAAAAAAACCAAACACCCCCTCAATATTTCAGAACAG
AAGAGTGCTGCCTGTGTTCCTCATAGGGCCACCCCTCCCCCCATGTTAACCCTTTAGTTACAAGATGGAG
GGAAGGTAGGGGCTGTGGACCCCACGTCTATGCTGAGACCCGAATGATAAATAGAGGCTGGTTGGCTGCA
GAAGGTGGGAAGAGGTACAAGCTGAAGTCACAACTCCTGGAAAGAGCCCGAGGCAAGAGAGCAGATGAAC
AAGTGAGACTATAGGGGGTTTAGGGAATAATCAATGAAGAGACTGAGGTCCATTGAGGTGGTTGAAAGAG
AAGAGCTTCTTTCTCCCCCAGCCCATCTTACAGATGGACAAACTAAGGCCCAGAGATAGCCACGGAGCTT
CCTAAAATCACTCAGCCAGGACTAGGACCCAGGCCATCTGACTCCCAGCCCAGAGGGGAGTCAGAAGTGG
GTGTGGAGCCTGCTCTGGGGAGGGGAGGGACAGCCACACTGACCATATCTGTCTGTCTGTCCTGCAGGCC
CGGGGAGAAGGAGAGATGTCTAAATCCCTATGGTTCAAAGGCGGGTAAGTAATGAAACTGTGAGCGGTGG
GAGGAAGATTTGAACCCCAAGTCCAAGGCTGGTTTGCGCCTGAGCCTCAGCCCAGAGAGGAGGAGGAGGG
GTTCCATTTAGACAGGGGACAGATTGAGCTTGGGCAAAATACGTCACTTTCATAAGGCACACAGCCATAC
GCAGACACAAAAGACAGTAATCTTTGAAGACAGGGCGGGGTGCGGTGGCTCATGCCTGTAATCCTAGCAC
TTTGGGAGGCCGAGGCGGGAGGATCATTTGAGGTCAGGAGTTCGAAACCAGCCTGGCCAACATGGTGAAA
CCCTGTCTCTACTAAAAACACAAAAATTAGCTGGGCATGCTGGCAGGCGCCTGTAATCCCAGCTACTTGG
GATGCTGAGGCAAGATAATTACTTGAACCCGAGAGGCAGAGGTTGTAGTGAGCTGAGATTGCACCACTGC
ACTCCAGCCTGGGAGACAGAGCGGGACACTGTCTCAAAAATAAACAAACAAATAAATAAATAAATAAATG
TAAGATAAAGCAGACACTATGAGGAACTAGACACTATTGGGAAATGTACACTATATATTTGTAATCCTGG
AGCAAAATTTTTGCGTTGATTCTATTTTTAAATCTCTTTGATTATTGCTACAGTTGTGGGCTCTTTAATT
AAAGATAGTTGTTTAGTCTTCAGTGGAGAATGTGGTCATGTCTAACCTCTTATGTTATGGATTTTCAGGT
TAGAGGATGCACAAGATATTTTAAAAATATGCTCAGGTTTTAAAATAGCAGCACGATAGTGACCTTCCTT
GTACGTCCTTGGCAAACAGGCAGGGAGACAGCAGCTGGGACTCCTAAATAAATGAGCAGGTACCCTAGCT
AGAGAGCCTTGGATGCGGGTCCTGAAATGCGGATTCCTGTGCACGTGATTTATGAAGGATGTGCTCTCGG
GAGAAACCTGTCAGACAAGGGGAGGCTGCCGAGAAAAGATGTGGTTTAGGAGTCTCTAAGCCTCAGTTGG
CTCCTGCAGAAAGCTTTGTAGGTTGAATAGCCCTGCAGGGCTTGTCTGGAGTCATTGGCTGCAGGCACCT
ATCTCACTGGCCAGTGAAAACCTTTTCGGGGCCAGGCTTGGTGGCTCACGCCTGTAATCCCAACACTTTG
GGAGGCTGAAGCAGGTGGATCGCCTGACGTCAGGAGTTCAAGACCAGCCTGGCCAACATGGCAAAATCCC
GTCTCTACTAAAATATACAAAAAATTAGCCAGGTGTGGTGGTGCATGCTTGTAATCCCAGCTATTTGGGA
GGCTGAGGCAGGAGAATCACTTGAGGCTAGGAGTTCAAGACCAGCCTGGGCAACATAGCGAGACCCCCAT
CTCTACAAAAGAATTTTTTTTTAATTAGCTATGTGTGGGCCGGGTGCAGTGGCTCATGCCTGTAATCCCA
GCACTTTGGGAGGCCTAGGCGGTTGATCACCTGAGGTCAGGAGTCTGAGACCAGCCTGGTCAACATGGTG
AAACCCCATATCTACGAAAAATACAGAAAATTAGCCAGGTGTCGTGGTGCGCACCTGTAATCCCAGCTAC
TTGGGAGGCTGAGATAGGAGAATTGCTTGAACCCAGGAGGCAGAGGTTGCAGTGAGCCAAGATCATACCA
TTGCATTCCAGCCTGGGCGACAGAGCGAGACTCCATCTCAAAAAAAAAAAAAAAAAAAAAATTTGCTGGG
TGTGGCGGTGTACACCTATGGTCCCAGCTACTCAGGAGGCTAAGGTGGGAGGATTGCTGTAGCCCAGGAG
GTGGAGGCTACAGTGAGCCGTGATTGTGCCACTGCACTCCAGCCTGGGTGACAGAGTGAGATCTTGTTTC
GAAAGATGGCTGGGCATGGTGGCTCACGCCTGTAATCTCAGCACTTTGGGAGGCCAAGGTGGGCGGATCG
CCTGAGGTCAGGAGTTCGAGACCAGCCTGGCCAACATGGTGAAAGCTTGTTTCTACTAAAAATACAAAAA
TTAGCCAGGCGTTGTGGTGGGCACCTGTAATCCCAGCTACTTGGGGAGCTGAGGCAGGAGAATTGCTTAA
ACCTGGGAGGCAGAGGTTGCAATGAGCCAAGAAAGGTTTCATCTATATTTTATTTTATTTTTGATACAGA
GTCTCGCTCTGTCACCCAGGCTGGATTGCAGTGGCGTGATCTCGACTCACTGCAACCTCTGCCTCCTGGT
TCAAGCAATTATCCTGCTTCAACCTCCTGAGTAGCTGGGACTACAGGCATGCACTACCATGCCTGGCTAA
TTTTTGTATTTTTAGTAGAGACGGGGTTTCACCATGTTGGTCAGGCTGGTCTCGAACTCCTAACCTCAGG
TGATCCGCCCGCCTCGGCCTCCCAAAGTGTTGGGATTATTACAGGCGTAAGCCACCACTCCTGGCCTCAT
TTATATTTTAGTATATTATTTAGATTTGCCTTAATTCACGTTTTCATTCAAGGTGGGCAATTACATCATC
TCCCCATTTTTCAGATGAGGAGGGTGAGGCCGGGTGTCTCCATTTCTTAGGACTGCTGTCACAAAGTACC
ACAGACGGGTAGCTTAAAACCACAGCAATGTATTCTCTCACAGTTCTGGAGGCCAGAAGTCTGAAATCAA
GGCATTGGTAGAGCCATGCTCCCTCTGAAATCTGTAGAAGAGAATTCCTCCCCTGCCTCTTCTAGCTTCT
AGTTTGCTTGAATTCCTTGGCCCCCGAGTATTGGTTTGTAGATACGTCGCTTTCTGCCTCTGCTGTCACA
GAGCCACCTTCTCCCCATGTATCTCTTCTTCTAAGGACACCAGTCATATTAGATTAAGTGTCTACCCTAC
TCCAGTATGACCTTATCTTAACTAATTACATCTGTGACAACCCCGTTTCCAAGTAAGGTCACCTTCTGAG
GTAGTGGGGGTTAGGGCATCAGCGTATCTTTTTTTTGAGGGACCCAATTCAACCCCTTAACACCTGGGGG
AGGTTGAATGTTCTCCCCAGGGCTCTATGTGAGGAATAGGGACATCTGGGCCCTCAGTGTCCCTCTGTGT
CTTTCAGCCCAGGGGGCGGTCTCATCATCATCGACAGCATGCCAGACATCCGCAAGAGGAAACCTATCCC
ACTCGTGAGCGACTTGGTAAGCACCCTCGCCCTTTACTCCCTGAGCAGAGCAGGCCTGCTATAAGCAGTG
CACAACCTGTGGAACTGCCCATGGTCAACCTGGGGTTGGGGGCCAGCTAGGCATTCTCTGAGACTAATGT
AGTCTGATTTCTACCCCCATGTGCTGCGTGGACACTTCAGGCCATGGTGAGTGATGGCGACATGGGCCTC
TTGGGATCTTCTCCCCAAACTCCCCCACTGGGATCTTCTAACAGGCCCCCAATTCATGGATTATGGGGGA
ACCTTGCCATGTCCCACTGTGTTGGATGCTGTGAGTCAGGGGTGCGGGGCTGCTCACTTTCCAGGGTCCC
TTCTAACTCAAAGCCTCTGTCACCTCCTAATTCTTTGTTGGGTCTGGCCCGGATGCTGTCGGTGACCTGT
TTCCAACTTGCGGTTGTGGCTCCAAAATGTGGCTGCTGGCCCCATCCGTGGCTGTGAACTCTCCTGGTGG
TGATCTCATGGATGTCAACCCCCCCGACTCCCTCTTCTGTGACCTCCCCAGTCCCTGGTCCAGTCCAGGA
AAGCGGGCATCACCTCGGCCTTGGCCTCCAGCACGTTGAACAACGAGGAGCTGGTGCGTGGGGCACCTCT
TTTTCCCCGGGTCAGGGATGGACTGGGCTAGGAGCTTCTGGAATTGTCCAAGCCTCTGTGGGTCCCCGGG
AAGAGGAAGGGTGTGTGTGTGACACAGACTGGCAGAAGGCAGCGTGGATTCCGACAGCGTGGAGGCAGGG
GAGGGCCTGGGACTTTACCGCGCTCAGTGATGGGGCGGAATGGGGCAGGACAGGGACAGCGCCAGGTTAG
GCGCCCCCAAACCGCACCCCATCCCTCCCTGCAGAAAAACCACGTTTACAAGAAGACCCTGCAAGCCTTA
ATCTACCCCATCTCGTGCACGACGCCACACAACTTCGAAGTGTGGACGGCCACCACGCCCACCTACTGCT
ACGAGTGCGAGGGGCTGCTGTGGGGCATCGCGAGGCAGGGCATGCGCTGCACCGAGTGCGGTGTCAAGTG
CCACGAGAAGTGCCAGGACCTGCTCAACGCCGACTGCCTGCAGCGTGAGCGCAGGGCCTGAGCGCAGGGC
CACGGGTTGGCGTGGGGAGCCCCGGGGCTTGCATGGCGGGGAAAGGCTGAAGGGCAAGGCGATGGGTGGG
GCACCATGGGGCGGGGCAAGGGGCGGGGCGTCGTGAGGCAGGGGAGGGACTCGCTGAAAGGGAGGGGTGG
GGTTAAAGGCGCAGGACTCCCTGGTGGAAGGGCTAGGGGGTCGAGGGGTGGGGAAAGAAGCTGTGAGGGT
GTCAGAGAGGGGTGGGGCAGGGCTCAGGGACTAGCTAGGTTGTGCAAAGGACAGGCTTAGTGAGAAGAGG
CAGCTCCCCTGGATAAAATGGGTTAGCTAGGGGTAGCTCTGAGAGGAAGAGGGGCTCAGAGAGGGAAGGG
GTTCAGACTGCGAGAGGACTGTGGAGGGGGGGATTGGAAGGGGGAGGGGACTCAGAGGAGGTGGGACCAA
GAGAAAGGGAGGGGTCTGAGAGATGGAAGGGGCTCAGGAATGTGGAGGGGACTCGCAGGGGTGGAGATCA
GAGACGAGGAGGGGATTCAGAGGAGTGGGGCTCAGAGAGGCAGCGGGACTCAGAGGGGTGGGACTCAGAG
AGGGGGCGGAGATTCAGAGGGGTGGGACGCAGAGGGGGCGGGGACTCAGAGGGGTGGGGCTTAGGGAAGA
GGCCTTAGAGGGGTGGGGCTCCAAAAACCAGTGGGGACTCCAAGGGGTGCGGGGCAGAGAAGGGCAGGAC
GCAGGGACATGGCGACTCAGAGGAGCATGGCTCCGAGAGGAGGTGGAAGGGTGAAGGGTCTGGGTGGGGG
GTACAGAAAGCAGGTAGGAAACCGGCTTCAGGGGGTGACACCTGAGTCCCCCCGGTTGATGAGTAGGGGC
GGGGCTCTCGCCTTGGTGAGGGGCCACTATGCAGGGCGCGCTGGGGTCAGCGCCGGCCTCGGGCGTCCTC
AGGGGCTGCGGAGAAGAGCTCCAAGCACGGGGCGGAGGACCGGACACAGAACATCATCATGGTGCTCAAG
GACCGCATGAAGATCCGGGAGCGCAACAAGCCCGAGATCTTCGAGCTCATCCAGGAGATCTTCGCGGTGA
CCAAGACGGCGCACACGCAGCAGATGAAGGCGGTCAAGCAGAGCGTGCTGGACGGCACGTCCAAGTGGTC
CGCCAAGATCAGCATCACCGGTGAGGGGGCGTGGGCCGCCATAGGGCCCCTCCTTCTCCACCCTCTGAGA
CCTGTCTCATGCCCTGTCCCATGGCCTGGTCCCTCTCCTTTGCAGCGCGCACTCACTCGGTCGGGTGCAT
TCCATCTCATCTCCGTGTGTGCACGCATATGGGGGGACATGCGTGCCTCCAAACGCCTGTTCATGGCACT
GTATCTGGGTGCGTCTTGGATGGTGCCCTCCAGAATGAGACTCCAAGAAGAGGAATTGAGGGTGCATGGT
TTATCTGGAAAGTGACCCCAGAAAGTCCTGGTGAGGGGCAAGGACTTGAGACAGGGATGGAAGGAGTCCT
GAAGGAGATGCCGTTGAGCAGACAATGCTGTGGGCAAGGGGGTCAGCCCATCCTGGGGCCACTGGGGACA
ATGTCGAGCATGCAGCTCAGTGTGGCCCCACTTAGGGGCGAGGATACTGGCTTTTCTCCACACCCAGCCT
CATGTGGGTTTCCCCATAAACAGACCTGAGGATGAGGATTTCAGGTTATGTGGGTTATCCAGGAAGTGAC
CCCAGGACGGAGCTGGGGTGGTAAGTCAGGGAGGGAAGGAACCCTGCAGGGGGCACCAACGAGCAGGAGA
TCTCGGGGGGCAATGGGTTCTTGTAAGTGTCTGACCTCAGGGAGTTCACACCTGAGAGAGGTCCTACCCA
TGCGCAAGGCAGCTGGGGCACCTTCCCATCCTTCATGGTTTGGGGACTCCTTCCCAGCACTTTGGGAGGC
TGAGGCTGAGGCAGGCGGATCACCTGAGGTCAGGAATTCGAGACCGACCTGGCCAACATGGTGAAACCCC
ATCTCTACTAAAAATGCAAAACGATTGGCCGGATGGGGTGGCACGTGCGGGTAATCCCAGCTACTTGGGA
GGCTGAGGCAGGAGAATTGCTTGAACCCGGGAGGCGGAGGTTGCACTCCAGCCTGGGTGACGAGCAAAAC
TCTGTCTCAAAAACAAACAAACAAACAAACAAACACACAAAAAACCTCTCTGGCAAGCCCAGCGTGCCCT
GCCATTGCTCTCCAGCCATGCTGCAGGTGTCCCAATTATCAGCCTGCAGCGTGTGGCAGTGACTGTCCCC
AGCATGCCTCGCTTCTTGAGTGCACACCTGTGTATGCCCTCTGTGTCTTCCAGTGGTCTGCGCCCAGGGC
TTGCAGGCAAAGGACAAGACAGGATCCAGTGACCCCTATGTCACCGTCCAGGTCGGGAAGACCAAGAAAC
GGACAAAAACCATCTATGGGAACCTCAACCCGGTGTGGGAGGAGAATTTCCACTTGTAAGTGCCTGGCTG
GGTCCCTGGCCCCATGTGGGGCATCCTGCTGGGGCAGCCAAAAGAGGGCTCCAAGGACAGGACTCACACT
GGCTGCCTGACCCCTGCCTCTGCCTCCTCCTCCCGCCACAGTGAATGTCACAATTCCTCCGACCGCATCA
AGGTGCGCGTCTGGGACGAGGATGACGACATCAAATCCCGCGTGAAACAGAGGTTCAAGAGGGAATCTGA
CGATTTCCTGGGGCAGACGATCATTGAGGTGCGGACGCTCAGCGGCGAGATGGACGTGTGGTACAACCTG
GGTGAGAGAAGCTGGGTTCTGAAGCGGGTGGGGACGGGTCCCAGGGATCCAGCCAGAGCATGGAGCCCAC
GAAGAGCAGAGAGATGTGTTCTCAGGAGGAGTTTGGTCGAGGAGCATAATCTGATGGGGGAGGCAGGGTC
TAGGAGCCCAGTTTGATGGGAGCAGGAAGCACCCAGGCCAGGAGGGTGAGCAGAGCCCAGGAACCTCTTA
GTTCAGCTTAGGAGGGAGGGTGGGGACAGAATCTCCATGTTCTAGTCTAATGGGGGAGGCAAGAAGCAAG
AACTGGGAATCCTAGTCTGAGGGGAGAGACAGAGACCTAGGAACTAAATCTAAGAAAGAAGATTGAGCTT
TAAGAATCCAGGCTGGGGCCAGGTATGGTGGCTCACGCCTGTAATCCCAGCACTTTGGGAAGCTGAGGCA
GGCAGATCACATGAGGTCAGGAGTTCGAGACCAGCCTGGCTAACATGGTGAAACCCCGTCTCCACTAAAA
ATACAAAAATTAGCCAGGCGTGGTGGTGGGTGCCTGTAGTCCCAGCTACTTGGGAGGCTGAGGCAGGAGA
ATCGCTTGAACCCAGGAAGCGGAGGTTGCAGTGAGCTGAGACTGCGCCACTGCACTCCAGCCTAGGCAAC
GAGAGCAAAACTCCCACTCAAAAAAAAAAAAAAAAGAATCCAGGCTGGGGGCGCCGGGCAGTGGCTCACA
TCTGTAATTAATCCTATCACTATGGGAGGCTGAGGCAGGCCAATCACTTGAGTCCAGGAGTTCAAGACCA
GCCTGGGCAACATAGTGAAACCCCATCTCTATAAAAGATACAAAAATTAGCTAGGCAGGGTGACACTCAC
CTGTGGTCCCACCTACTCTGGAGGCTAAGGTGGGAGGATCCCCTGAGCCTGAGAGGTAAAGGCTGCAGTG
AGCTGTGATCACACTGCTGCACTCCAGCCCAGGGAACAGAGTGAGACCCTTTCTCAAAAAACAAATATGG
CCGGGTGCAGTGTCTCACGCCTATAATCCCAACAGTTTGGGACGGCTGAGGCAGGCAGATCACTTGAGGT
CAGGAGTTTGAGACCAGATTGTCCAACATGGCGAGATCAAGCCCCTGCACTCCAGCCTGGGCAACAAAGG
AAGACTCCGTCTCAAAAAAAAAAAAAAAAAACAAAAGAAAAAGAAAAAAAAAATGGAATCCAGGCTGGGC
ATGGTGGCTCCCACCTGTTATCCCAACATATTGGTTGGGTGGCTGAGCTGGGAGGGTCACTTGAGCCCAG
GAGTTCGAGACCAGCCTGGGCAACATAACAAGGCCTTTGTCTCCATTAAAAAAAAAAAAATAGCTGGGTG
TGGTGATGCGTGCCTGTAGTCCCAGCCACTTGGGAGGCTGAGGTGGAAGCATCATTTGTACCTGGGAGGT
TGAAGCTGCAGTGAGCTATGATCATGCCACTGCACTCCAGCCTGGGCGATAGAGCAAGACTCTGTGTCCC
CCCGACCCCCCACCACAAGAAAAGAATCCAGTCTAAAGAGGGAGGTATTGTTTGGGAATCCTAATCTGAG
ATGGCTGCTACAGATCAAAGAACCCTGTGTAAGGAAGTTGAGTAGAATCTGGGGAGTCCAATCTAATGGA
GGAAGCAGGGTGTATGAGCCTAATCTGAGGGGGTAGAAAGGACCCAGCCTGAGAGAGGTGAGCAGAGCCC
GGAAACCTCTTAGATTGGAGGACATCTAGATATCCCATTCTGATGGGGGAGGTGGGAGACAAGACCAGGG
GGTCCTAGTCTGAGGAGAGAGGCACAGACCCAGGAACAGTGTCTAAGATGTGTAGTCTCCGGGGAGCATG
ATCTGATGGGGGAGGCAGGGCCTGGGAGCTCAGACTGGGAAAGAAGTGGGGCCTAGGGCTGCTAGTCTAA
GGAGGGAGGCACATAGCCAGGAACTTAATCTGAGTGAAGAAGCAAGGTCTGGGATCCTTGTTTGAAGGAA
GAGACAGGGCTTAAGGAATTCTAGCCTAAACAGGGAGACTCAAGCCTAGGGAGCCAATCAGAAGAGAGAC
ACCAAAGCCTTAGTTTCCGGAAGTCTGGGAGGACTACAGAATCGTGTATCTATTAAAAATTGCCTTGGGA
GGCCGAGGCGGGCGGATCACCTGAGGTCAGGAGTTCGAGACCAGCCTGGCCAACATGGTGAAACCTCGTC
TCTACTAAAAATAAAAAATTAGGTGGGTGTGATGGCACATGCCTGTAATCCCAGCTACTCGGGAGGCTGA
GGCAAGACAATTGCTTGAACTCAGGAGGTGGAGATTGCAGTGAGCCAAGATTGCGCCACCGCACTCTAAC
CTGGACCACAGATCTCAAAAAAAAATAAAATAAAAAAAAATTTGCCCATGGCTGGCCGGGCGCGGTGACT
CACGCCTGTAATTCCAGCACTTTGGGAGGCTGAGGTAGGTGGATCACCTGAGGTAAGGAGTTCGAGACCA
GCCTGGCCAACATGGTGAAACCCCGTCTCTACTAAAAGTATAAAAATTAGCTGGGCACGGTGGCAGGCAC
CTGTAATTCCAGCTAGTTGGGAGGCTGAGACAGGAGAATCGCTTGAACCCAGGAGGTGGAGGTTGCAGTG
AGCTGAGATCGTGCCACTGCACTCCAGCCTGGGTGACAAGAGCAAGACTCCCTCTTAAAAAAAAAAAAAT
TGCCCACTGCTATAGTGGGAAGTTCTGACACTGTCTTCTTTTTCTGCCCTGCAGACAAGCGAACTGACAA
ATCTGCCGTGTCGGGTGCCATCCGGCTCCACATCAGTGTGGAGATCAAAGGCGAGGAGAAGGTGGCCCCG
TACCATGTCCAGTACACCTGTCTGCATGAGGTGAGGGTCATTGCTCGGCCCCTCCCATGCCACTTCCACT
CACCATTCCTGCCTGCCCAGCTCTTCCTCTTTCTGGCCACACCATCCACACTCTCCTGGCCCTCTGAGAC
TGCCCGCCATGCCATTCCCTTTACCTGGAAAACTCCTCCCTATCCATCAAAGTCCAGATTCAGGGTCACC
TCCTCTGGGAAGCCCACCTTGGCCTCCAGGTTGACTCTCACTACTCATCATCAGGTTCTTCCTTCTATTC
CAGCCCTAACCACTCAGGATTGGGCCGTTTGTGTCTGGGTATGTCTCTTCCAGCTGCCTGGGTTTCCTGG
AAAGAACTCTTATCCCCAGGAACTAGTTTGTTGAATAAATGCTGGTGAATGAATGAATGATTGAACAGAT
GAATGAGTGATGAGTAGATAAAAGGATGGATGGAGAGATGGGTGAGTACATGGATGGATAGATGGATGAG
TTGGTGGGTAGATTCGTGGCTAGATGGATGATGGATGGATGGACAGATGGATGGATATATGATTGAACTA
TTGAAAGTATAGATGTATGGATGGGTGAATTTGGGGGTAATTGTTAGATGATGGATGAGTATAGATGAAT
GATGGATGGATAACTTGATGAGTGGATAGATAGATTGCTGGATAGATGATTGACTGGGTGGATAGATGAA
ATGTTGGATGAGCAGATTAAGTTGTATTGGATGGGATGGATGGAAGTGTGGTTGAGTTATTAGAAGGAAG
ATTGAGTAGATAGGTGAATTTGTTGATAGTCAGATGGGTAGATAGGTAGATGGATGGATGGATGGATGGA
TGTATAGGCAGATGGACAAATGGATGAATGGGTGGGTGGATGAATGGAAGGATGTGTGGTTGAACTATTG
CAAGTATTGATAATTGGGTTCATAATTTCTGAATATTTAGATGGATGGTTGTGAGTGGCTGGTGGACAGA
CGAAAAATGGATGGTTGGATAAATTGATGGGTGGATGGATGGTTGGTTGTATGAAAGAATGAATGATTGG
GTAGGTGGATTAAGTTGCGGATCAATGTATGGGATGGATGAATGGATGGATGGATGGATGTGTGGTTGAA
TTACTGAAAGGTTGGAAGAGTGGATGGGTGAAATTTGGGGTAGTTAGATGGGTGGGTGTGTGGATGGATA
AAAGAGTAGATGAATGAATTAATGAATAAACAGGCAGATGGATGATGTAAGCTGCCCCAGACCCTGGGAC
CTCTGACCCCCGGCGACCCCTTGCACTCTCCATGACACTTTCTCTCCCATGGTGGCAGAACCTGTTCCAC
TTCGTGACCGACGTGCAGAACAATGGGGTCGTGAAGATCCCAGATGCCAAGGGTGACGATGCCTGGAAGG
TTTACTACGATGAGACAGCCCAGGAGATTGTGGACGAGTTTGCCATGCGCTACGGCGTCGAGTCCATCTA
CCAAGCCATGACGTGAGACTGCAGGCTGGGGTGCAGGAAACAAGAGGGAGGTCACCAAGTGGGCACTGGC
AGGTCTGGGGTGGGAGGGATTCTCAGGGGGAAGACCCAGAGAGGAAGTGGGCGTGGTGGTGGTGGGTTCC
GTAATTCCCCTGGAGATCAGGAGAGCGCTGTGATTGACACCTGGAGTGACATGGAAGAGAGGGAAGACTT
AGAAGCAGAGATAGAGGCTGGGCACAGTGGCTCAAGCCTGTAATCCCAGCATTTTAGGAGGCTGAGGTGG
GCGGATCACTCCAGGTCCAGGAGTACAAGACCAGCCTGGCCAACATGGTGAAACCCCATCTCTACTAAAA
ATACAAAAGTTATCCAGGTGTGGTGGCTTGGGCCTGTAATCCCAGCTACTCGGGAAGTTAGGGTGGGAGA
ATTGCTTGAACCTGGGGGGCAGGGGTTGCAGTGAGCCGAGATTGCGCCACTAAACTCCAGCCTGGGTGAC
AGAGTGAGACTCCATCTCAAAAAAAAAAAAAAAAGAAAGAAAGAAAAAAAGAAGCAGAATTAGAGCAACT
CAAATGGATATAAAGATGGAGGGGAGAGGAGATGCGGAGACCCAAAGGACAGAATCAGGCAGGGGATGGT
TTAGGAGGTGACAGAGACCCACTCACAGATGCTATCTTGGAGGCATTCTAGGGGGGATCTTGGTCCATCC
TGGCTGGACCCCTGGCCTAGTGCCTCCTGCTCCCTCTCTCCAGCCACTTTGCCTGCCTCTCCTCCAAGTA
TATGTGCCCAGGGGTGCCTGCCGTCATGAGCACCCTGCTCGCCAACATCAATGCCTACTACGCACACACC
ACCGCCTCCACCAACGTGTCTGCCTCCGACCGCTTCGCCGCCTCCAACTTTGGGGTCAGTCCTGGGGACT
GGGATTGGAGAATTAGGGAGAGCCCAACTTTATGCCGGTCAGATCTGATGTGACTTCTGGGTCCCCAGTC
TAGACCTGATTGCTGTGTGACTTTCCACCAGTCACTTCCCGTCTCTGAGCCTCCATTTATTCCTCTGTAG
AATGCTATAGAATGGAGGTCGGGCGCAGTGGCTCATGCCTGTAATCCCAGCACTTTGGGAGGCCGAGGCT
GGAGAATCACTTGAGGCCAGGAGTTCGAGACCAGCCTGGCCAACACGGTGAAAACCCGTCTCTTCTAAAA
ATCCAAAAGAAAATTAGCTGGGTGTGGTGGCCTGTGCCTGTAGTCCCAGCCACTTGGGAGGCTGAGGCAG
GAGAATTGCTTGAGCCTAGGAGTCGGAGGTTGCAGTGAGCTGAGACTGCGCCACTACACTCCAGCCTGGG
CAACAGAGCAAGACTCTGTCTCAAAACAAAAAAAAAAATGCCATAGAATGGAAATGGACATTATGAGGGT
GGTGGATGAGGTATACTTACTGTACAGTAGGCGCTCAGCCACTATGGCGGCCATGTCCTGCCTCCATCCA
TCCTCCCTCCTCCCCTTCCTCCTCAGAAAGAGCGCTTCGTGAAACTCCTGGACCAGCTGCATAACTCCCT
GCGGATTGACCTCTCCATGTACCGGGTAGGAAGTGTGTGCATGAGAATTTGCTCACCCCACGCACATGTG
TGTGCCTGGAGGGGAAAGCTACCAGCAGGTGTGTGCATGGGAGGACCCATCTTCGCATGTACGATGTGTT
CCTCCAGCATGTGTGCACCTGCATGGGCACTACCTGCCGTAATGTGCTTGGTGCATCCAAGCAGATCCCC
AGTTGCTTCGAGGTGCTCTAATCAACCCCCAGCCTGAAACCGGGGAGCCTTTGTATCCCCCTGAAAAGCA
GTCTCCCATCCTCCCCACGCCTTCTTCCATAACCCCACATCTCTTCCTGCTCCCAACAGAATAACTTCCC
AGCCAGCAGCCCGGAGAGACTCCAGGACCTCAAATCCACTGTGGACCTTCTCACCAGCATCACCTTCTTT
CGGATGAAGGTAGGAAAGGATCCAGTTGCCTTTGCTCTCCAAGGAGGGTTCTCTAGGATCCCCAGCTCCC
AGGACAAGTGACATTCCTCCTGGAGAGTGGCAGCCTTTCTCAGTAACCTTTTCCTTTCATACCACTTCAT
TTCCTTAAACCCAAAATGACTCGCAGTCACTTCCTGGGGACCTCCTGTCACAGCTGTGGCCGCCTATGCC
TCTCTCCCTCCCTTCAGGTACAAGAACTCCAGAGCCCGCCCCGAGCCAGCCAGGTGGTAAAGGACTGTGT
GAAAGCCTGCCTTAATTCTACCTACGAGTACATCTTCAATAACTGCCATGAACTGTACAGCCGGGAGTAC
CAGACAGACCCGGTGAGACTCCAGATGGGTCAGAAGGGAACAGTGATGCCAACTAGACACAGGCTTCAGT
CCCAGCCCTGCCCCTTCCCGAGGGATTTAACTCTTCTGAACCTCAGTATCTTCTTCTATAAAATGGGGTC
ATTAACCCACATCTCAGGATTAGGGAGAAGAGTTCATGAGCTGTAAGTTTAGATAAAATATAGTCGGCAC
TCAGTACATTTGGTTGCGTAGTTGAGTTTGGCATAAAAAAAAATTTTTTTTTTTTGAGATAAAGTTTCAC
TCTTGCTTCCCAGGCTGGAGTGCAGTGGTGTGATCTCAGCTCACTGCAACCTCTGCCTCCCAGGTTTAAA
TGATTCTCCTGCCTCAGACTCCCGAGTAGCTGGGATTACAGGTGCCCACCACCACACCTAGTTAATTTTT
TGTATTTTTAGTAAAAACGGGGTTTCACCATGTTGGCCAGGCTGGTCTCGAACTCCTTGACCTCAGGTGA
TCCACCTGCCTCGGCCTCCCAAAGTACTGAGATTATAGGCGTGAGCCACCACTCCCAGCCTGGCATAAAA
ATTTATGTCTGGCATACAGTCACTGTTCAGTAAATGTTGAGTTGAATTGAATTTATTTATTTATTTTGAG
ATGGGGTCTTGCTCTGTTGCCCAGGCTGGAGTGCAGTGATGCAATCATGGCTCACTGCAGCCTCGACCTC
CCAGGCTCAAGTGATCCTCCCACCTCAGCCTCCCAAGTAGCTGGGACTATAGGCACGCACCACCATGACA
GGCTAGTTTTTGTATTTTTGTGGAGATGGCATCTCACTATGTTGCCCAGGCTGATCTCCTGGGCTCAAGC
GATCCTCCTGCCTCAGCCTCCCAAAGTGCTGGGATTACAGGCATGAGCCACCACCTGTAAGGAGCTTATA
GCCTATTATGAGGGAAAGATGTACAAACTGGAAATTTACAAACAGGGATGGTTAGTATTGAGATAGTGGG
ATCTCAGGAGGCTGTGGGAGCTGGAGGCAGCCCCTACCCAGGTTCTGATAGGATATAACACATAAGCTGT
GTTCAGTTCAATTTGATTTCATTCTGTTCCTTTACATTCAGTTGACTAGAGTTGGAATCAGTCTCACTCT
CATTTGAATTGAGTTGAGGTGAACTGGATTAAATTAGGCTATAAGGAGCTTATAGCCTATTATGAGGGAA
AGATGTACAAACTGGAAATTTACAAACAGGGATGGTTAGTATTGAGATAGTGGGATCTCAGGAGGCTGTG
GGAGCTGGAGGCAGCCCCTACCCAGGTTCTGATAGGATATAACACATAAGCTGTGTTCAGTTCAATTTGA
TTTCATTCTGTTCCTTTACATTCAGTTGACTAGAGTTGGAATCAGTCTCACTCTCATTTGAATTGAGTTG
AGGTGAACTGGATTAAATTAGATTGTTTAGTTCAACCATAATCAGTTTAGTTATATTTATTTCTATTTGG
TTGACTTAAGTTAAAATTGTTCTTGACGGCCAGGCACAGTGGCTCACGCCTGTAATCCCAGCACTTTGAG
AGGCCGAGGTGGGTGGATCATGAGGTCAGGAGATCGAGACCATCCTGGCTAACACAGTGAAACCCCGTCT
CTACTAAAAATACAAAAAAATTAGCCGGGCGTGGTGGCGGGCACCTGTAGTCCCAGATACTTGGGAGGCT
GAGGCAGGAGAATGGTGTGAACCTGGGAGGCAGAGCTTGCAGTGAGCTGAGATCGCATCACTGCACTCCG
GCCTGGGTGACAGAGCGAGACTCCGTCTCAAAAAAAAAAAAAAAATTGTTCTTGACAAATTAGTGAGCCA
GGCTGGGCTCAGTGGCTCACACCTGTAATGTCAGCACTTTAGGAGGCTGAGGCAGGAGGATCCCTTGAGG
CCAGGAGCTCGAGACCAGTTTGGGCAACATAGCGAGACCCTATCTTTGCAAAATATACAAAAATTAGCCA
GGTGTGGTGGCGCACCTGTAGTCCTGGCTACTTTGGAGGCTGAGGTGGAAGGATCACTTGAGCCCAGGAG
TTCGAGTCTATGGTGAGCCATGATTGCACCACTGCGCTCCAGCCTGGGTGACAGAATGAGACTCTGTCTC
TTGAAAGAAAAAAAAAAAAAATAGAGAGAAGAACAAGTTGGTTGATTGCATTGAGTTTTATTGAGTACAA
ATTCATTCATTTCCATTTAGTTCATTTACATTCAAGTCAAGTTGTTCTCAACTGGGTTAAATTGTCTTGA
GTTAGGATGAACTGGGTTGTTGGATTAGATGCAATTGATTTCAGTTGAATTGGGTTGAATTGGGCTAATT
TCAGCTGATTATATTCATTTCCATTGATTAACAAAGGTGGGTTGAATTGCCCTGCGTATGTTCAGTTGGG
TTTAGATAGGTAGAATCGGATTGGGTTGACTTGGGTAGAGTTGAGTTCGAGTCGATTTATTTCCATTATT
TTGATTTGGGTTGATGTTGATCTTGGACAAGTTGATTAGGTTCAATCCAACGGAATTCATTTGAGTTTAA
TTCAATCCAGTTAGGTTGAATTGCACTCCATTCAGTGTGATTGGCCCAAGTTCAGTTCCATTGATTTAAC
TCAGAGGAAGAACTTACTTCCGTTAAGCTAAGTGGGCTTAATTTGCATTCATTTCATTTCCATTTGGTTT
AGTGGAGCTGGGTTTGTTCAACTAAATTTGGTTTCATTTATTTCCATTCACTTTTAGAGTGGAGTCTAAA
AATCTTAAGCTTTATCAAGCTGCATTGACATGAGCTCTGTTTGGGCCAGTTTATGAGTTCAATTCCTCTC
TATATAGATTTCTTACCTGAACCATACACATGATGATGGGATGCATTCCTCTAAAACCTCTTGTTCTTCA
GTAATAAACTGACCGAGGCACCAACCTCTGGACCCCCTATAACCGAGGTCTCCACTGTCCATTTCAGGCC
AAGAAGGGGGAAGTTCTCCCAGAGGAACAGGGGCCCAGCATCAAGAACCTCGACTTCTGGTCCAAGCTGA
TTACCCTCATAGTGTCCATCATTGAGGAAGACAAGAATTCCTACACTCCCTGCCTCAACCAGTGAGTTGT
GTATCTGTGTAGTTAATTATCTGGGTGTCATCGTGTGTATGTATACAACTTTGTGTCTTGTGTGTCAAGA
TGATTTTGTCTAATTACACAAATGTGCACCTGGGGGTAATTGTGGGTCTGATTATACCTGTGCAAAATTC
AAATATGGGTACAATTTGCAAACATAAACATAAAATTGTGTGTATGTTCACAATCACTGGTATAATCAGA
CATGTATTTTGTATGTATTCATTTGCATGCATAACTGTGTGTACATATGTGTATAATTTTTTATGTGTAT
GTAGGTTAATTAATAATAGTGTTGCTCAAGTATTACTATGTGCTACCCTAATACATATCATACATTTAAA
TTGCATTATATCTTATATGTATGGATGTAAGTAATTTTGTATATTGTTGTGGTCAGTGGGTTTTGGGTTT
AATTTGTATATGTGTAAAATCATTGAATGCAATTCAACGTACAATATTGTACAATATACAATATTGTGTA
TTGTGTATGAATATGGGCACAGTTTTCATTTGTGGATTTATATGTCTGTAGAATTTTCATCTGTGTTAGC
TGAATGAGCTACTGTGTTCTATTTCTCTGCCAGATGAGTGCTAGAGAGGACTCTAGCTTTGGGGCGTTAA
CATATCCTGGTTAGGGAATTAAGATCAGGGATTTGGATGGATTGAAGGGTGGGTGGATGAATGGATGGAT
TAATAAACAGATGGGGTCAGGCACAGTGGCTCACGCCTGTAATCCCAGCACTTTGGGAGGCCAAGGTGGG
TGGATCATTTGAGGTCAGGAGTTTGAGACCAGCCTGGCCAACATGGTGAAACCCTATCTTTACTAAAAAT
ACAAAAATTGGCCACGCGCGGTGGCTCACCCCTGTAATCCCAGCACTTTGGGAGGCCGAGGCAGGCGGAT
CATGAGGTCAGATGGAGACCATCCTGGCTAACATGGTGAAACCTTGTCTCTACTAAAAATACAAAAATTA
GCCGGGAGTGGTGGTGGGCGCCTGTAGTCCCAGCTACTCGGGAGGCTGAGGCAGGAGAATGGCGTGAACC
CAGGAGGTGGAGCTTGCAGTGAGCCGAGATTGGCCACTGCACTCCAGCCTGGGCAACAGAGTGAGACTCC
GTCTCAAAAAAAACAGAAAAAACAAAACAAAAATTAACCGGGCGTGGTGGTACACGTCTGTAATCCCAGC
TAGTGGGAGACTGAGGCAGGAAAATTGCTTGAACCCAGGAGGCAGAGGTTGCAGTGAGTCGAGATTGCGC
CACTGCACTCCAGCCTGGGGTGACAGAGCGAGACTCCGTCTCTAAATAAATAAGTAAATAACAGATGGAC
AGATAGATTGGTAGAAGGATGACTGTGTGGATAAAGGAACGGATGGGCCAGGTGTGGCAGCTCACGCTTG
TAATCCCAGCACTTTGGGAGGCCGAGGCGGGAGGATCACCTGAGGTCAGGAGTTTGAGACCAGCCTGGCC
AACATGGCAAAACCCCATCTCTACTAAAAATATAAAAAATTAGCCGGGCATAGTGGCTCGCGCCTGTAAT
CCCAGCTACTTGGGAGGCTGAGGCACGAGAATCATTTGAGCCCGGGTGGTGGAGGTTGCAGTGAGCTGAA
ATCGCACCACTGCACTCCAGCCTGGGTGACAGAGTGAGACTCCGTCTCTAAATAAAAAAAAAGGAATGGA
TGGATGGGTGGACAGATGGATGGGTAAATGGATGGGTGGATAGCTGCATAAGTGAGTAAATAAATGGATA
GAGGGATGGATGGATAGGTGGATGGATGAATAGATAGATGGGTCATTTAATGGGTGGGTGGATAGTTGTA
TGAGTGGGCGAATGAATGGATGGAAGGACAGATAAATAGGTGGATGATGGAAGGATGGACGGATGGATGG
ATGAACAGATAGACGGGTAGGTGGCTGGATGGACAGTTGGATGAATGGGTGGAGGGATAGATGGATGAAC
AGGTAGATGAGTGGTTGAGTGGATGGATGAATAGTTGGATGAATGAGCAAATGAATGGATGGAGGGATGG
ATGGATAGATGAATAGATAGATGGATAGGTGAACGGATGGGTGGATAGTTGCATAATTAGGTGAATGAAT
GGAAAGAGGGATGGATGGATGGATGGGTGGATAGATGAATAGATAGATGGGTAGGTGGATGGATGGATAG
TTGGATGAATGGGTAGAGGGATAGATGGATGAACAGATAGATGGGTGGTTGAGTGGATGGATGAATAGTT
GGATGAATGAGTGAATGAGTGGATGGAGGGATGGATGGATAAATAGATAGATGGATAGGTGAATGAATGG
GTGGATAGTTGCATAAGTAGGTGAATGAATGGATAGAGGGATGGATGGATGGGTGGATGGATGAATAGAT
AGATGGGTAGGTGGAGGGATAGATAGTTGCATGAGTGGGTGAATGAATTGATGAAGGGATGGTTGGATGG
GTGGATAATGGGTGGATGGATGAATAGATAGATGGGTACATGGATGGGTGGACAGTTGCATGAGTGGGTG
AATGAATGGATGGAAGGATGGATGAATGGATGGAGGAATAGATAAATGAGTTGTTGAATGTATGGGTGGA
TAGTTGCATGAGTGGATGAATGAATGAATGGAGGGACAAATGGATGAGTGGGTGGAGGCTCAAAGGGAAA
CAAAGCCTTAGTGCAGAGTCAGTCAGCAGTGGCTATGAGGGAACCTGAACCCAAATCCCTCTACCCTACC
CTACACTCCTCTGTGGGGCAGGGAAGGACAAAGCAGCCCATCCCATCTGCCTTCTGCCTGCCAAAGTTTT
ATACTTCCATGACTTTGCCAGGTTTCCCCAGGAGCTGAATGTGGGTAAAATCAGCGCTGAAGTGATGTGG
AATCTGTTTGCCCAAGACATGAAGTACGCCATGGAGGGTGAGTTTCCCACCCCAGTGTTCCCCAGCCTGG
CCACACCAGGTGGCTGTACCTCTGTGTCCCCTCCAAGGCTGTGAGCACCCTATAATACCCTCATGCCTGA
GTTGGAAATCAGTGATGGCCAGAGTAGGCAGCCTCTCTGCCGAGACAGAGGGTGGCAGACCCTTCCAGCT
GTGCCATCCTCTTTGTCCCTGGCCAGAGCACGACAAGCATCGTCTATGCAAGAGTGCCGACTACATGAAC
CTCCACTTCAAGGTGAAATGGCTCTACAATGAGTATGTGACGGAACTTCCCGCCTTCAAGGACCGCGTGC
CTGAGTACCCTGCGTAAGTCCCCTGCCCCAGGCCCAACCCAAGCCAGAACTGTAGCACTGACAGCTAGAA
GGAAAGCCAGAGAGGAAGACTGACCTACTTAGGGTTACCTGGGCATCAGGGGCATGGGTGGGGCTTGAAT
CCAGTGTTCTCCACTCTCAGCCATTGAGCAGTTGACTCCTACAGCCCAGAAAATTCAGTCAACAGCAACA
GGAACCATGTGACTTTCTCATTTTATCCTTTTGACAATTCTATGAGTTAAGGTGGGATCATTTTAATTCC
TATTTTGCGGAGGAGAAATTGGAGAGTTAAAGATGCCAAATGGCCAGGCATGGTGGCACATGCCTGTAGC
CCCGGCAATCTGGGAGACTGAGGCAGGACTTGCTTGAGCCCAGGAGTTCGAGACCAGCCTGGAAAAGATA
GTCAGATCCCATCTCTAAAATTAATGAATGGGTCAGGCACAGTGGCTTGTGCCTGTAATCCCAGGACTTT
GGGAGGCCGAAGTGGGCAGATCACCTGAGGTCAGGAGTTCGAGACCAGCATAGCCAATAGGGCGAAACCC
CAGCTCTACAAAAATACAAAAATTGGCCGGGCGCAGTGGCTCATGCCTGTAATCTCAGCACTTTGGGAGG
CCAAGGTGGGTGGATCACCAGAGGTCAGGAGTTTGAGACCGGCCTGGCCAACATGGTGAGACCCTGTCTC
TACTAAAAATACAAAAATTAGCCAGGTGTGGTGGCAGATGCCTGTAATCCCAGCTACTTGGGAGGCTGAG
GCATGAGAATTGCTTGAACCCAGGAGGCGGAGGTTGCAGTGAGCCGAGATTGCACCACTGGATTCCAGCT
GGGGCAACAAAGTGAGACTCCGTCTCAAAAACAAAGAAAAAAATTATCCGGGTGTGATGGCACACACCTG
TAGTCCCAGCTACTCGGGAGGCCGAAGCAGGAGGATCGCTTGAACTCAGGAGGCAGAGGTTGCAGTAAGC
GGAAATCGTGTCACTGCACTCCAGCCTGGGCGACAGAGTGAGACTCCATTTCAAAAAATAAATAAATATA
AAAATAAAATGAATGAGTGAATAAATGCCACATTCCTTGCCCACGGTGACTCCCAGTACATGACAGACCC
GAGATTTGAACTCGGGACATCTGATTCCTGGGCTGTGTGTTGGAGGATAGTACGGTGCCGCCGGTTCCTG
AGAATTGTGGTGTCGTTCTCTGCTCCCAGCAGCACAGGAGGATGGCTAGTCTTCTGTTGTACAAGAAACG
AAAGCTCTGGCTGGATGCGGTGGCTCATTCCTGTAATCCCAACATTTTGGGAGGCCAAGGTGGGAGGATC
ACTTGAGGCCAGGAGTTCAAGGCCTGCCTGGGCAACACAGGGAAATCCTGTCTCTACAAAGAAGACAAAA
AAAAAAATTAGCCGGGCATGGTGGCATGTGCCTGTAGTGCCAGCTATTCGGGAAGCTGAGGTGGGAGGAT
TGCTTGAACCCAGCAGGTCAAGGCTGCAGTGAGCTGTGATCGCACCACTGCACTTTAGCCTGGGCAACAG
AACAAGACCCAGGAAGGAAGGAAGGAAGGAAGAAGGAAGGAAGGAAGGAAGGGAGGGAGGGAGGGAAGGA
AGGAAAGGAGGGAGGGAGGGAGGGAGGGAGAAAACAAGAGCTCAGAGAATAAACCCACTTGCCATGGGCA
GTTTATCTGAAGAAGGAAGGAAGGAAGGAAGGAAGGAAGGAGGGAGGGAGGGAGGGAAGGATGGACGGAG
GACTCAGAGAGGACTCAGAGAATAAACCCACTTGCCATGGGCAGCTTATCTGAACTAGGATTCCCACAGG
CCTGGGCCCTGTTGTAAAAGGGGCCACTCAATCTATTTCATGAGCAAAGTAACTGAGGTTCAGTGCAGGA
GGCGAGCAGAGGAGCAGCCACTCAAGGCTGCAGGGAGAGCTCCAGGCAGAGCAGGAGCAGCCATAGCTCC
ACTCGTTAGTGGGTCAGAACCAGGCGGCGCAGAGGTTGAGGAGGACAAGAGGTGGCAGAGCCCCACCCCG
GCTCTTCATTCCAGATGGTTTGAACCCTTCGTCATCCAGTGGCTGGATGAGAATGAGGAGGTGTCCCGGG
ATTTCCTGCACGGTGCCCTGGAGCGAGACAAGAAGGATGGGGTAAGAAGGCCCACCCACAACCAAGTTCC
TAATCTTCCCACTGGGGTCAATTTAGGTGGAAATTTGCCCTTGAGATGAGGCCAGTCTCTGGTAGGTGTC
CGCCATCTTTGTCTTGCTCATGGTTTTTAAAATCTTTTGTTCATTCATCAACTACAAAATGTGAGTTCCA
TCAAGCCGGACACCTCCAGGCACCCCATGGCTTCTGCGTCCACCTCTAGAGTTGACTCTAAGTCTACCTC
GCCCTCTGTCCATCTCACCCCGGGGAAATTGGTCTCTGGCTCTCCCCGTGGAGAGTGGGAGAGTTGGGCT
CTAGGGATTCTCTGGTGCTGATCTCCTCCTTCCTCTTGGCCTCCTTCCCCCTGCAGTTCCAGCAGACCTC
AGAGCATGCCCTATTCTCCTGCTCCGTGGTGGATGTTTTCTCCCAACTCAACCAGAGCTTTGAAATCATC
AAGAAACTCGAGTGTCCCGACCCTCAGATCGTGGGGCACTACATGAGGCGCTTTGCCAAGGTGTGGGAGA
GTGGGTAGGGTTGGGGCTAGGGGACAGGGTCAGGGTTAGGGAATTGAAGTCTGAGTTGGAGATGGGCTAA
GGTTGAGGTTGAGGTTGGAGTTGGTGATGTCATTGAGGTTGGGATTGAAGTTGGAGTTTGGGTTGAGGTT
GGAGTTGAAGTTTGGGTTGAGGTTGAGGTTGAGATGAGGTTGGAGTTGAGGTTTGAATGGAGGTTGGAGT
TGGGGTTAGGGTTTAAATTGAGATTGGAGTTGAGATTGGGGTTGGAGTTGAGGTGTAGGGTAAGGTTGGA
GTTGAGGTGTGGGTTGAGATCAGGGTTGGAATTGAGGTCATTAGTGCGAGTTGGCAATGCTGTTTGGTTG
GAGTTTGAGGCTGTGCTGGGTCTTGATTTGGAATTGAGATTGGAGTGAGTGCTGAGATTGATGCACAATT
TGAGTGAGTCTTGGGGTGAGTTTGAGGGTTGGGATCATTGGGGTTGAGGTTTGATTCATTGGCATTAGGC
TGTGTTGACCTTGGGAATTACAAGTAGGAATTGGCTTTTGATTTGAGATCAGGTCATCTTGTGGTTGAGG
CTGGGTTAATATGATGCTTGGTTGCTCTGAAAAAGAGCTGGGTCTACATAAAATGATCATCTTCATCGAG
CGGTATCTCCTCTAGGTCTTGATGGTCATCTTGAGTGGAGAATTTGCCTAGCCCAAAGGCCCATCTTAGG
TCCTAATAGGGTTTGGGTTCATCACTGATCTCACTAGTGGCCTTGATGGGACGACTGGCGCCTGCCTTGG
TGCCAGCCTCTCCTCTCACCCACACTCTGTGTCTTCCCCAGACCATCAGTAATGTGCTCCTCCAGTATGC
AGACATCATCTCCAAGGACTTTGCCTCCTACTGCTCCAAGGAGAAGGAGAAAGTGGTGACCTGAGGGGGC
CCCTGACCTATCTCCCTCCTCAGCCCCATTCAGCATCCCCAGGACTCCCTTCTCCCCAGGGCCCTGACAC
CTGGGGCTATGTATGTCCACCTGTGTGTGATCTGACTGGATTGTCTGTGTGTTTAGAGGTGCTTGTTTGA
ATGTGTGTGATCAGAGTGTATCTAGTTGTGTGTGTCTGAGTGTATGGGAAGTAAGTGCATGTGTCTGACT
GTGTATTGTCTGTGTTGACTGGTATACTCTGCGTGTGTGTGTGTGTGTGAGTCTGTGCACATCTGAGTGT
GCGTGTCTGATCATGTTTTGTCTGGCTTTGTTAACAGGGTATGGCATGAATGTGTGTGTGAGTCCAACTG
TGCACGTCTGAGTGTGCGTGTGTGATTGTGTTTCGTCTGGTTGTGTTAACTGGGTATAACATGAGTGTGT
GTATCTGTGTGATTGTGTTTCGTCTGGCTGTGTTAACTGGGTATAACATGAATGTGTGTATCTGTGTGTG
TCTGTACATATCTGAGTGTGGGTATCTAGCCATGTTTCATCTGTGTTAACCGGGTATAGTGTGAGTGTAT
GTATGTGTGAGTATGTGTGTTCAACTATACAACACATCTAGGTGTGTGTCTCATATTTTATCTAGCTGTG
TTAACTGGTTATAGTGTGAGTGTGTGTATGCTTGTGTGTGTGTCCAGCTGTGCATGTCTGGGTGTGTGTG
ACCGTGTTTTGTCTGGCTGAGTTAACTGCATATAGTGTGAGTGTGTGTGTGTGTTTGTGCACGTCTGGGT
GGGTGTGTCTCACCAGGTTTGGTATGGCGGTGTTGAATGTGCGTGGTGTGAGTGTGTGTCTATGGCTGTG
TCTGGCTGTGCATGTCTGAGTGTGTGTATCTGTGGGGGGGGTCTCGTGTGTATATCTCGGGGTGAGGGCA
CACGCCTTCAGTGTGTCACTGTCTGGCTGTTGCAAATGAAGCCTTCAGGGTGTGAGCTGCAAAGCTTTGG
GCTCTTGCAGGAAACTCTGCTACTCCCAGAGTTCCCAGCCCCTCTCATGGACCCCCCACCAGGCCTGGGG
TCGTGGGATTACTTGAGTCCTCCTTGCCCCCACCAGTCCCCAGAGATGCCCGCCCCAACAACACGCCCAG
GAACCTGAGCACAAAAGCTCCCTTCCATTTGCAGGTGACCCCATGGGACCCCAGCTTCCTCCAGACCCAG
ACCCTGAAACCATCCCAAGGTCCCTGGCGGTGGGTAGGGGAGGCTCTGGGGAGAGGTCCTGAGGCTTGAC
TCCCCTCTCTGTTCCCACCCACAGCCCTGCATTCTCATGAATAACACTCAACAGCTACGAGTTCAGCTGG
AGAAGATGTTCGAAGCCATGGGAGGAAAGGAGGTGAGGCAGGGCCGAGGCAGGGCTGGAGAAGGGATGGG
GCACCCCCACCACTGTGTCCCTGCAGGCCCCTGAGCTCATATGCCACCAGGCCTGGGGATGAATTCCAGC
TCAACCATGGCCTTAAACCCAGGCTTGCAGCATAACGGTCTGTGCCTCAGTTTCCCCTGCAGCCCCTTGG
GATCCCTTTCCCTGGGTCAGATGAGAAAATGCCCAGTACATGTGGATACTTACTGAACCTGGCCTGGTGT
CCCTTCTCCCTCCTTAGCTGGATGCTGAAGCCAGTGACATCCTGAAGGAGCTTCAGGTGAAACTCAATAA
CGTCTTGGATGAGCTCAGCCGGGTGTTTGCTACCAGGTGGGAGCATCTCCAGGGCTGGGGGCAGTGGGGA
GGGAACAGCCTTCAGCCCTCTAAGCCTGGGGCATCTGGAGGCTCAGCAGATGTAGAGGTGAAGATTGCAG
GCTCAGAGTTAGACAAGGCTGAGTTCAAATCTTGGCTTTACTGCTTACTAGCTGTGTGGCCTTGGGTGAG
TCACTTAACTTCTCTGAGCCTTATTTTTTTTTTTATTGCTCTGTAAATTTGGGGTGATGACAATAATAGT
ACCTGGGTAGGATTTTGAGAATGAAATGAACAATGCGGGCAAATGTCTTATCCCTTTGCCTGGTTCTGCA
GTTACTATTTTCCACTCTTTTTTTTTTTTTGAGACGAAGTCTCGTTCTGTCACCAGGCTGGAGTGCAGTG
GCGCGATCTCGGCTCACTGCACCCTCCACTTCCTGGGTTCAAGTAGCTGGGACTACAGGTGCATGCCACC
ACACCCAGCTAATTTTTGTATTTTTAGTAGAGACAGGGTTTCACCATGTTGGCCAGGGTGGTCTCGATCT
CTTGACCTCGTGATCCGCCCGCCTTGGCCTCCCAAAGTGCTTGGATTAAAGGCGTGAGCCACCGCATCTG
GCCTGTTCTCCATTCTTGCTCGGTTATGTGCTGTGATGCAAATGATATGACCTCAAGGACCACAGATCAG
CATCTGTGTCCTCATCCAGCCCTTCCTGTGGTCTGAGCCCTTCCCTTCTGTGTCCCCAGCTTCCAGCCGC
ACATTGAAGAGTGTGTCAAACAGATGGGTGACATCCTTAGCCAGGTTAAGGGCACAGGCAATGTGCCAGC
CAGTGCCTGCAGCAGCGTGGCCCAGGACGCGGACAATGTGTTGCAGCCCATCATGGACCTGCTGGACAGC
AAGTGAGCTGGGCCAGGTGGACTGGGCTGGCCGGGGAGGGGCTGGGGCAGGGCCATAGAGACCCAGGGAC
TGGCTGGAGAGGGTGTGCAGGGAATGTTTGCTGGGTGGTCACTGGATAGTTGGATGGCTGAGTAGATGGT
GGCATGAAGGGGATGGAAAGTTGGGTGGGTAAATAATTGGAAGGATTGGTGAGTAAATATTTGTGTGGAT
GAATGAATGGTGGATGAATAGGTGACATGAGGGATGGGTAGGTGGGTGGACGCATGGATTAAAAGATAAA
TGTTTAGAAGGATAGAGGGAGGATAGATGTTTTAAAAAGATGGATGGGTGGATAGTTGGGTAAATATTTG
GATGGATTAATAAATGTTTTGATGCATAGATGGATTGATGCTTAGATGGATGGATGTCTGGCAGGCTGGC
TTGATGGATGGTTGGATGGACGATGGATGGATGGATGGATGGATGGATGGATGGATGGATGGATAAATGG
TTGGATATGTGGATAAATATTTGGATGGATGAATAAATGTTTTGATGCATAAATGGATGGACAGATGGGT
GGATGGATGAATAAATGGATGGGTGGGTAGATGGATGGATAGATAGGTGGATAAATATTTGGATGAATGA
ATAAATGTTTTGATGTATAGATGGATTGATGCATAGATGTCTGTCTGGCTGGCTGGATGGATGGATGGAT
GAATGGATGGATGGATAGGTCAATAGATATTTAGATCATTGAATAAATGTTTTAATGCATAAATGGATGG
ACAGATGGGTGGATGGATGAATGGATGGTTGGATGGGTGGGTGGGTAGATGGATGGATGCTGGATAGATA
TAGGTGGATAAATATTTGGATGGATAAATAAATGTTTTGATGCATAAATGGTTGGACAGATGGGGGGGTG
GGTGGATGAGTGGGTCGATGGATGGATGGATTAATAGGTGAGAAATATTTGGATGGATGAATAAATGTTT
TGATGCATAGATGGATGGACAGATGGATGAATGGACAGGTGGATGGATAGATAAATGGATAAGTGGATGG
ATGGGTAGAAGGATGCATGGAAGGATGGATGGATGAACAGATGGATGGATGGATGGATAGAAAAAGAAAT
AGAGAATTAAGGACCACTGGGGGAGGGATGGATTGGTGGGTGACTGGATCAGTTGGTGGATGGATCTTGG
TGGACTGCCTGTCTCCTTCAACCCCTATCCATCCAACCACAATCTCTTTGCTGTTTTCCCTTTCAAGTCT
GCCCTCCTCTGACCATTCCCCTCCTGTTCCTCTTGGGCATGGCCTTCTCCCTCATAGTCCCTGATCTCCA
TCCTTCCTGTTTCGGTTCATCCCCCACACTGTTCTTTCAAACATGAAAGTCTGGCTGTGTCTCCCTCTTG
AACACTCCATGGCTCCCCACTACCCCCATCCTGATAAAACCCAAGCCTTCCTCCCAGACATTGGGGCCCC
TTCCCATCTGGTCCCTGCTGACTAGTCCAACCACCACTCACTCTTCTCTTCATGCATCAGATATCATAGC
CCCATCAAACCACCCAGGGGTCCCTGTACAGGCTGTGGGCCCTCTTTCCTATCTGTGGAATGCCTTGCCC
ACCTGTTAAGGGAAGGTGATCTGTGGGTGGGGGCGAGCTGGGCCCTCTCTCAGACCTGCCCCTCGTCCCC
AGCCTGACCCTCTTTGCCAAAATCTGTGAGAAGACTGTGCTGAAGCGAGTGCTGAAGGAGCTGTGGAAGC
TGGTTATGAACACCATGGAGAAAACCATCGTCCTGCCGCCCCTCACTGACCAGACGGTGAGACCTGCAGG
GGGCCCGAGGGGACATTTAGGCCACCTCCCTGGCGAGAGCCCAGAAAACTTGGTGCCTAGAGGCTGGGGG
GTAAGAACAAAGGCATCCGGTCTCAGAGAGGTCATCCAGGCTCAAGGGCCATTCAAGGGTCATGGAAGCC
ACCAGAGGTCAGTGGGGGGCCATTCAGAGGTCAGAGAGTTCACACAGGGGTTAAAGATCATCGAAGAGTT
AAAGAGGTCATTCAGAGTCCATTGTATTTTCTCTGGGGTCAAAGACATCAGGTAGAGTCAAGAGACCACT
AAAGTCATAGAGGTCACATGTAGGTCAAAATAGCTTTCAAAGGTCAGAGGTCATCTAGAAAACAGGTCAA
TTTTGGGATCAAGGTTATCCTTGAGCCACGGAAGGCATAGACATTGGCCAGGCACCGTGGCTCACGCCTG
CAATCCCAGCACTTTGGGAGGCTCGAGGCGGGCAGATTGCTTGAGGTCAGGAGTTCGAGACCAGCCTGGG
CAACATGGTGAAATCTCGTCTCTACTAAAAATACAAAAATTAGCTGGGTGTGATCCTGTGATCCTGGCTT
CTTGGGAAACTGAGGCACGAAAACTGTTTGAACCTAAGAGGTAGAGGCTGCAGTGAGCTGAGATGGCGCC
ACTGCACACTCCAGCCTGGGCAACAGAACGAGACCCTTTCTCAAAAAAACAAAAAAAAAAAAGAGAAGGC
ATAGACATCAATGGGTTTTTCTAAGGGCTTTGGGGCTCATTCAAAAGCCAAAGAGGGGAGACACCATGGA
CTTCAGACATCAGAGATCAATAGACCAAGGGGCTCCTGGAAGTCAGCCAGAGGTCATGGAGGGCATCCAA
GGTCAGAGAGGTCAAGCAGAGATCATTGGCCATCCAGAGACCAGGACCGATAGACCAGAGGCCATAATCT
CTAGGGACCAAGGGGACCCAAACATCTGTAGAGGGATGTAGCTGGGCGTGGGTCCCTCCGGGAGGCATCA
AGAACCTGAGGACAGCAATGGCATGTGGGCTGTGGGCACATTAGGGAGTGGGGAGGTCTTTCCCAGGTGG
ACCACGGAGGCTTGGGGAGGGTGAGCGGGGGCCATAGATGGGCGACGATGGTCGGGGAGGCTCGGACTTC
CCATCCTTCCACCTCTTCTGGGCTGGGCTGGGGCAGGGGTCTTGCCCCTCCGTGCTGCCATCTCTGCCCC
CTCCCTGGCCTTCCCGGGAGACTCATGGTACCTTATTCCCCCCTCCCCCACCGCCCCGCCCCCATCACGG
ATGACAGATGATCGTAAGTAGAGTCCCGTCTGTCCGTCTGTCTGTCCGTCCTCTCTGTGTCTGGACTCGG
CCCACCGCGTCGCTGCACCCCTCTCCCCACCCACCCAGCGCCCCCTCCTCACCCACCATCCTCCCTCCCG
CCTAGATGTGCCTGTGTCTGTCTGGGGCGGGGCATGGGGAGGGGGCGGGGCTGGGAGGGAGGGAGACCAC
GCCCACCCTCCCTGTCCCACTGAAGTCCAGAGGAGCCCCACTACCCACTCCCAAACCCCAAGCCCAGCTC
CTCCTTCGCAGCGGACCTGCCGTCTAAGGGTAGAATCCAACCTTGCTCCCGTGCCCAGATGTCACCAGAC
GCCCTGAAGCTAGAAAGAGGATCAACCCCAGGATTCCTCTGACCTCCTCTTTGGGCTCTGAACTGTCAGC
AGCTTCCCAGAATGCAGGTCCTCACCATGAGGGTCTCTACTTCTGATTCCTCTGAAATTCCATGCTCCAG
ACCATTCCTTGGTACCCCACCACCCTGTCTACCCAACTCCCGCCATCCACACCGACACACACCAATCCCA
GACATACACCCAGGTGCCCAAGTGGGAGACTTTTGTCATGGCTTTGATGTCAGGTAGACTTGGGGTCAAA
TCCTAGCCCAGCCACTGTCTCTCTCTGTGACCTTGGGAAGTCACTTGACCTCCCAGCCTCTGTTTCCTTG
TCTGGAAAATGGGGCTCATTGCATCACCTCCCTGAGGGTAGTTCTAAGAACCAAATGGCATGATGCATTA
AAATACTCATCTCAGTATTGCCTGAACCCAGGAAGCAGAGGTTGCAGTGAGCCGAGATCGCGCCACTGCA
CTCTCAGTGACCGGCCCAGAGGCAGAGCTAATGGTGGTGGTGGTGGTGGTGGTGGTGGTGGTGATATGCA
GACATGTGTAAGAGCAGAGACAAAACAACTTCAGATTTTCCCTTTGTGGGCAAGACAAGGCTGTTACAAG
ATTTCACTTTGTTAAAAGTTTACAAGGGCTCTTACTTCCTGGTAGCTGCAGCCTGGGTCTTTGGCTATAA
CCACTTGGTGGCAGCATACCCAACCACAGGCAATGCATTTGTAGGCAGGGGGAATAAAGGAATTCGCATT
TAATGACTCCCCATATGCCAGGCCTTCTCATAGCTCCTGTTTCAGCTCACCTTCATGATAACCCTTTGAG
TGAGGTGGATATTATTATCCCCATTTTGCAGATGAGGAAAGCTGAGGCTCAGAGAGGAGTCACTTGTTCA
AGGTCATATAGTAAATATATGCCAGAATCAGGATTCAAATTCAGATCTGTCTGAATATGCAGCCCATTTG
CTTTCCTGTTAACCCAAGAACCAAGAGTCTCATGTGTACAGTAAATGTAAATGGATTATATTTTGGCAGT
GGCTTTTTTCCCTTCTTTCTGACTTTCTTTAGCTCTGTATCTGTATCTTTCTCTCTCTTTCCTGTCTCTG
CCTCTCACACCATTTAAGAGTGAGTCACTAAAGCGCATGCACGTGTGTGCACACACACAGACACACACAC
AAACACACTATCCCCATGCAACCCAGTCACCAGTACCCTGTGCAGTGTGGATTCCCCCATCCCCATGAAT
ATTCCTGTGAGCATCCCAGCTTCCCACCCTGCACGACTTGCCAGGTTGATCACCCTGTGACATTATCTCT
GCAGGGGAACCTCTTGAGAAAACATGGCAAGGGATTAGAAAAGGTAATGAGGGCCTCGTCTCACTGAGGC
CCTGAGCTCCAGTGTCTGGGCATGTGTGTGCATGTGTGTGCGTCTGTGCACCTGGGCAGGAAGGGCAGCT
GGGGGTCGTGGGTAGGAGATAGCTCTCCTAACCAAGTCCTGCAAAACCAGAAGTCAGAGATGAAGTGATT
CTCCAGGGACAGAACGTGCAGTGTGTGTGTCCACATGTGAACTAATGTGTGTGCACAAACAGCTCCCATC
CTCCCACAGCAATACCCACAGGGCTCATCTGAGCCCTGGGCTCAGCTGAAATTCTGTGCCTTCTTGAGGA
GCCTACACGCTAGATCACCACCCTGGACTGAGACGTGTTGTTTTTTACGTAGATTTCTTTTTGTTGCTGT
GCTAATGGGTGTTTGTTGTTAAGGAGATGGGAGCTACAGCAACACGCAAGCAGCTGTCTTGCTGTACACT
TGTGCAGAAGATATCGTTGTACATTTGTGTCTACAAATGGAGTCTTAGCTTTGTGAATGTGGGTCACAGG
GGTTCCGTACTCTCTCTGCCAGCCCTTTGCGGGGCACATTCAGTGTCCCAGGCACCCTATATGTGGATGT
GAATCCGTGTGGGGTCAAGAAGGGTGTGTGGGTTTTTGTGTGTGATATAATTGTGTACGTGCCTGCAAGT
GTTTAGGAGTGCACGTATGTAGTTTAGTGTATGGGTTCTTCAGCACTGAGTGAACTTACAGTATATGTTA
ATATGTTTTTTGTAGACTTGGGTGCTGGGATATAGGGGCAGCCCCTTAGGGAATGGAGGAGGAGACAGAT
TCCTAGCCCTGTCTCATCTCCCCACCTCCCCAGGTGCCCCATCCTGGGGCAGGCTGACTGTGGGATGAGG
CTGCTTCCAGCCTCCTGGGTACCCCGAGAGTGTGCTGGCAGTCAAGGAGTCTGACCTCTAAACCCCTGCT
AGGGGACCAGAGGAGGTGCTAGCTGGGGAGGGGGCCCACCATTAGACCCAGGACCTGGGGAGGAGCTCCT
GGGCTGGGAAGTGAGACTGTGCTTTGAGGGGGCAGGGAAGTCCCACTGAGTATCCTCCCAACAGTCACCA
GAACTCTGACCTCCACCTGATTCTCTCCACAGGGCAGGGTGAAATTGCCAAGCCACTCAGACGTAAGACC
GGCCTCTCACTGTCTGTCTGGCTCCCATCCCACTCCCCCCCACCCCACCCCTGTGCCCCCTTCCACCTCT
GTGGTTCGTGCCGTCCGAGGGCGTACTGTGAACCTGTCTGTTGGTCTCTTCTTCCTTTGTCTGTCTGGCT
TGCTCGTGGGGGGTGGTGGGTGGGGGGTTGGTGGGTAGGGCAGAGGGTACCCAGGAAGGCCCCTGAGCCC
TGCCAGACTACAGGTGGGTGCAGCAGGGGAAACTGAGGCCCAGTCCGCTGGACCCCTGAGTAACTGTGCC
TTGTAAGAACCTCAGGATCACAGACAAGAAATAAGAATAGTAACAGCCAAGATGATAATAGTAATACCTC
TAGGAATGACCAGATGGCCAGCTGGTAGACTATGAACATAATTCCAAATATTTCCACAGTAAATGGGCCC
CCTCCCCACCTAGCCTCACCCAGGACCCCCAGACCACCCCAGGACCCAGTAAGACAAGACTTGTTACCTC
AATACTCAGCCCCACCCTGTCCCCCAAGTTACCATGGGTATCCCTGTTAGCCACAGTGGGAATTGAGGCT
CAGAGCATTCAGGGACAAGCCTGGGGCCATCCAGCAGTGACAGAGGAGGCTAGACACATGCCTTCCAGGC
TGGGACCCCGTCTCCCTTAGCATTGCCCGTGACTTTGAGACCAAGTGATAATTAGCGAACACTGAGCAAA
GACTTGTTCAGTCCAGACTAAGCCACAAAGTGGATATAATTTTCCTCATCTAGATGGGAAAACTGAGGCT
CAGAGAGGTTAAGTGCCTTGCCCAGGGCCACACAGCAGGACTGCAATTTGAACCCAGGCCCATCTGGCTT
GTCAGAGCCATCACCACATCACCATCTTGCTAGTGTCTGTTTGGGGGTGGGGTGGAGGACGGATATGGAG
GTGTTTGTATAGAGGGAGGATTTTTCCTTTAAAAGGTCAGACTGGCTGGCAATGTGGCTCACGCCTGTAA
TCCCAGCACTTTGGGAGGCTGAGGTAGGAGGATCGATTGAGCCCAGGAATTCAAGACCAGCCTGGACAAC
ACAACAAGACCCCATCTCTATGAAAAAAATTTCAAAAACTAGCTGGATATGGTGGCTCACACGTGTAGTC
CCAGCTACTGGGGAGGCGGGAGGATTGCTTGAGCCCAGGAGTTTGAGGCTGCAATGAGCTATGATCATGC
CACTGCACTCCAGCCTGGGCAACAGAGGGAGACTCTGTCTCAAAAAAAAAAAAAAAGAAAAGAAAAATCA
GACTGAAATTCCTCCCAGATTACAGGGGAAATTTCCCTGCAGAGGGGATGCTCCTTGAGGAGGAGGAAGT
GTTTGACCAGAGCCATTTTCTGCAACCTCAGCCTAGCCTATTGTGTTCCCAAAGCTCCCTTTCCCTGGAA
ACCCTGCCCACACCCACAAACACCACCGTCTGAGGCTCCCCCAACCCACTGCCCCTCACAGTCTGGTCCC
TGTCTGTTTCTCCTGTTACACAAGCCTGGGTGGGGCTGAGAGGGTGGGGGACTATCCTCACAGGACCGGG
ATTTTCAGGGACAAGGTCACCACATCCTCCTCAGTTCTTCCTGGGGAATTCCAGACCTCTGTCCCTGGGA
CACAATTATTGCCCTGCCCAGAGCCTTTGGGCAAGAACCAAGATGGTCTCTGGGGCTGGAGTGTCAGCTG
TAGCACCCCTGCCCTCACCCAGCTGTATGTTGTTACCTGCAGGGAACCCAGATGATCTTCAATGCAGCCA
AGGAGCTGGGTCAGCTGTCCAAACTCAAGGTGAGGAGTGGGGATAGATTATGGCGTGAGGGACTGCCCCC
CAAACTCATGTGGCTTCCAGGTTGCCACCATCCTGGGGGAGGGGTCCCAGGATGATGACATGCATTACAG
GCTCAAAGGACAGAAACTCTACTCACTGAAGCACAAGATATTTGCTCCCCAGTGTGTCAGAAAGATGTGA
ATGGTTCAGCTTCAGGTACAGCTTGATCAAGAGGGCAAGTCATTTATTCAACCAATATTTTTTGAGCACC
TGCTGTGTACCAGTCACATATTGGGTGCTGGGATGATGAAAACATAGACCCTCAGCCCAGCTCCTCCAAG
TTGAACAGGGATGAGACCCATAGACACAGATTCCAAAGGTGGAGCCCACTCCACCCACATGGCCCCTCTC
CCCATCCATCCAGGATCACATGGTACGAGAAGAAGCCAAGAGCTTGACCCCAAAGCAGTGCGCGGTTGTT
GAGTTGGCCCTGGACACCATCAAGGTGAGAGGCCCTACCCCTCCCCATCCCAGGTGGGGGATGTAGAGGG
TGTGGCTGCTGTGGTCATGGACTTAGGCTCGCCTCGGACCCACATTAATTTGTTCAATTTATGGACACTC
CCTCGTTCCCCTGTCTGTGCCAGAGCTGGGCTGGCTAATGCTGGAGCCTTAAGGAGCCCCAGGTCGGGGA
AGACTGCTGGATACAGACATTCTCAGCCTTGTAGGATTAGGCTTGGGGTAGAGGGAAGGATGTGGGCTGG
GGGAGCCCAAAGAGTGAGAAGGTGTAGAGGGAAGGATGTAAAGGACCTCGTGCCAGGCATTTCTTGAGGT
GTAGAAGGGTAGAGGGAAGGATGGGGGCTGGGCGAGCCCAAGGTGGGAGAAGCCAGGCCTTCCTGGAAGA
CGGCATGTGAGAGGTGGGTTCTGAGGAATGCATAGGAGTTCACTAGGCAAAGCAAAGGAAGAGGTGAGAA
AGTAGTAGCGGGAAGAATTGGGAGTGGGGACAGCAGGGCTACCCAGCCCTATGAGTGGGTAGAGGTTCCC
ACCATCCTCACCTCTCCCCACCCTGTCCTCCCCAGCAATATTTCCACGCGGGTGGCGTGGGCCTCAAGAA
GACCTTCCTGGAGAAGAGCCCGGACCTGCAATCCTTGCGCTATGCCCTGTCGCTCTACACGCAGGCCACC
GACCTGCTAATCAAGACCTTTGTACAGACGCAATCGGCCCAGGGTAAGGGTACCCAGACCGCATCACCCT
TTCCCGCTCCGCGGAGACAGCCCCGCCCCAAGCCTGAACGGCTGCCATTGGCTCACTCCCCTGTGACGTC
ATGGATGGGGCGGAATTGACATCTCTGCACCAAGTCCAGGGACCAATGAGCGATCAAAGGTTCATGGGGG
CGGGGCCTCTGACGTCACAGGCCTGGAGGTGATCGTCAACCCCTGGGAAAATCTGTGATGTCCTGGGGCC
AGACAATGATTAACAGCTCTGGGATGGAATGTCTTTGACATCAAGGACCAGGGTAGGATTGATGACTCTT
AGGTGTGGAACCTGTGACGTCGTAGGGCCACAAGGTGATTGTCATCTCCTGGGAAAATCAATGGTGTCCC
AGGGCCAATGAATGATTGTCAGCTCCTGAGGGACCTGGACAGTCATGGGTCCAGGGAATCGTTGACAGCT
CTGGGAGACTTCAGGAGTGATTGACAGCTCAGGATGGGCAGGACCTCTGATGTCAAGGGGTCAAGGAATG
ATTGGCAGGTCCATGGGTGGGAACTTTGACGTCATGGTCCCAGAGAGTGGTTGAACTCCCCTGAGGAGGG
ACCTCTGTGATGTCATGGGGCTAAGGAATGATTGACAGCTCCGAGGGCGGGAGGGACCTCGACTTGGTGG
GGGAGGGTGGACGGAGAGGGAGTTCCCATTTCTCAAACTCTCTCTTTCTCTCTGTATTTTGCTTCGGTCT
CCTGTCCCTCTCTGAGCGCACACACACGGCAGCTGGGGCTGGATGGAGGGCAGGATTCCCTGGGGACCCC
CCCTTTTCAAAATTGGTCCCGACAAGGGGAAGAAGTTTGGCGGCTTGAACTCAGGAGGGGGAGGGAGAAT
CGCCATCGACACCCAGCCTGGGGCGAGGGGAGCAAAGCCCTGTGGCCTCCAACGGCCCCCTTCTCCCCCG
ATCCAGCCAGCCCAAGGGCCCTCCGTGGGCGCTGGGTCTCTGAGCTCAGCTTCCCTTCCAGTCCTGGCTG
CTTTTAGTGTCCCTGGTGTCCCTGGAGCAGGAGGCTGTCTGTCTCTCCGTCTGTCTGCAGCTGACGTTTC
TCTCTCTCTTGTTTTTCCTCCCTCTCACTCGCCGTCCCCTCTCCGCGCCTCCCCTGCCCCCTCCACACAC
ACTCCTCCATCCTCACCTCTCACTGGCTCGTCTCTCCCCGCTCCCCATGCTTCCCACCTCTCTCCCCCTT
CCACCCCCACCCCCAATTAGTCCATGGTGGAAAAGGTACTAGGTTTACCCTTAGTGAAGACATTTATCCT
GAGAAGGGTACGTGCCTCCGCCTCCCGCCCGCCGCCCGCGCCTGGTGCCCGGTGCCTGCGCCCCTCCTGG
CCCCGGCTAGTAAAAGGATCGTCCTTCTGCCTCAAGGGCCTGCAGGGCCGCCGCCCAGGCCTGAGCGGCT
TGGAGGGCCCAGGCGGGTGAGGGGGAAGGACGATTTGCTGGTTCTACATGGGGAAGAAGCGATTCCAGTG
GGAATCCCAGAATCCATTTCTCCTGATATGACATATTTTTTAAGCCAGATCCTGGGACCCCGGGACAGGC
ACCTGTCAGGTTAAGAGCTGGGCTCTGATTCCAGCCCAGCCACTCCACGGGTAGGCACCTTGGGCAAGAA
TCTGTCTCCCTGAGCCTCAGTTTTCTCATCTGTAAAATGGGGACAGTCAAGGACCCTACTTCATAGGATT
ATTGTTATTGATATTATTATTTAGAGACAAGGTCTCCTTCTGTCACCTAGGCTGGAATGCAGTGGTGCAA
TCACTGCTCACTGCAGCCTCCCTCCTGTGCTCAAGTGATCCTCCTGCCCCAGCCTCCCGAGGAGCTGGGA
CTACAGGCACACGTCACATGGCTAGCTAATTTTTAATTTTTTTTGTAGAGAGGTGGCCTCACTTTGTTGC
CCAGGCTGATCTCAGTGAATACTTATCAGGCTCTAAGGCTGAGCAGGACCTTGTGCCAGGCATTTCTTGA
AGAATCTGTGTTTGGTTTTGTTGTTGTTGTTTGAGACAGAGTCTCACTCTGTCACCCAGGCTGGAATGCA
GTGGGGAAATCTCAGCTCACTGCAACCTCCGTCCGCCTCCTGGTTTCAAGCGATTCTTGTGCCTCAGCCT
CCTGAGTAGCTGGGATTACAGGCCCGCACCACCACGCCTGGCTAATTTTTGTATTTTTAGTAGAGACGGG
GTTTTGCCATGTTGGCCAGGCTGGTCTTGAACTCTTGGCCTCAAGTGGTCCACCCATCTTGGCCTCGGCC
TTCCAAAGTGCTAGGATGACAGGTGTAATCCCAGCGCCTGGCCATTTTGTTTTTAATTTGAGGCAGACGC
TGTCACCCAGGCTGGAGTGCAATGGCGAAATCTCAGGTCACTGCAGCCGCCACCTCCTGGGCTATGTTGC
CCAGGCTGGTCTCGAACTCCTGGCCTCAAATGATCCTCCCACCTTGGCCTCCCAAAGTGCTGGGATTACA
GGCATGAGCCTCCATGCCTAGCCTTCTTGGGGAAACTGAAACAGATTTCTTTAACTGCCCCACGAGGGGT
ACATCATTCCACAGATGAGATACTGAGAGCCAAAGGAGATTCAACCACTTGTCCGAGGAACCTTCCCCTG
CAGGGGTCCCGGGACTTGGGGAGTGGGGAGTGGGGTTGGAAGAAGCACTGAGAGGCCCAGGAAAGACCCT
TCCCCGCGAAGCCAGCAAAGCACGGGATGGGGGCACCCAGCACTGCCTCCCTCCCCCCTTCTTACTAGCT
TTGGCCTTGGTCTGACCCCTCACCTGACCCACCACTGTCCAATTCCCCCTGGCACCCCCGGCACCCCAGT
CTGGCCCGGATTGAGCTTGCTATGAAGCATCGGCCTGGGGGAGGGCGGGGCGGCTGAGCATGTGACTGGT
GGCGGGGGGTGGGGGTGGAATGGGAGCCTGAGGCAACAAGGAAGACGGCCTGGGGCGTGCAGCAAAGACC
GGGGCCACATACCCCAGTACTGGGGTGATGGGGGTCTCCAGGCCCCTGTGCTCCCCAACCCTCAGTCACT
TCCTGTCCAGCGTGAGGTAACCCAATCAGCGTTTGACTGACCCAACCTCCCACCCTCACCTCTGACTTTC
CCCTTCTTCTCCCCGTGCATGGGGTGGTCCAGGAGTGGGGAGTTGCCTGGTGGGGGGTGGGGAGGGGTTG
GTGGAGGTGCTGGCGGTCACAGGTCCACAGGCCCCACAGGATGCCCTTTTGAGTCACAGAAGGCGGGACC
GGGAGAGATTCTTTTAAGCTGCCCCATCAGTGATCCCCATTGGCACCATGGAGGACACGGCTGGTGGCCC
CAAACAGAAGCCCACCACACCATGATTCAAGCAGACAAGGGAACTTTTTGACTCCATAATGGAAAAATTA
GGGGTTGAGGCCTGGCGCGGTGGTGCATGCCTGTAATCCCAGCACTTTGGGAGGCCAAGTTGGGCGGATC
ACTTGAAGTCAGGAGTTTGAGACCAGCCTGGCCAACATGGTGAAACCCCATCTCTACTAAAAATAACAAA
AATTAGCCAGGCGTGTTGGTGGATGCCTGTAATCCCAGCTACTCAGGAGGCTGAGGCAGGAGAATCGCTT
TAACCTGGGAGGCGGAGGTTGCAGTGAGCCGAGATGGTGCCATTACACTCCAGCCTGGTGACACAGCAAG
ACTCTATCTCAAAAACAAAAAAGAAGGCCAGGTGCGGTGGCTCATGCCTGTAATCCCAGCACTTTGGGAG
GCCGAGGCGGGCAGATCACCAGGTCAAGAGATCGAGACCATCCTGGCCAACATGGTGAAACCCCGTCTCA
AAACTACAAAAATTAGCTGGGTGTGGTGGTGTGCACCTGTAGTCCCAGCTACTCAGGAGGCTGAGGCAGG
AGAATCGCTTGAACCTGGGAGGCCGAGGTTGCAGTGAGCCGAGATTTCGCCATTGCACTCCAGCCTGGGC
GACAAAGCAAGACTCCATCTCAAAAAAAAGAAAAAAAAAAAAAAAAAAAAAAGAAACTTAAGATTCTGCA
CTAGAAGGGAAATAGGGGTAAGGAAAATAAAAGATGCCGCTATGAGCTGTCACATCAGGAGGGATGAGGA
TTCTGGGCCAGGTGATGCAATGAGGTGACCTGTGGGGCTGGGTCAGAGTTAACAAGGGCAAGAATGGGGT
GAGTGGGGAGAGGTAATAGTAACCTATTGGAAAGGGCCACTATTGGAAGGGGTGTTGCAGGTCACAAAAA
CCACTTTGATTATCTCAGAGTGAGGTGGGAGCCATGGAAGAGTTTGGTTTTGCTTTGTTTTGTTTTTTGA
GACAGTCTTGCTCTGTCACCCAGACTGAAGTGCAGTGGCGGGATCATAGCTCACTGCAGCCTCAACCAGC
TGGGATTAAGCGATCCTCCTGCCTCAGCCTCCTAGATAGCTGGGACTACAGGTGCATGCATCACCACACC
TGGCTAATTTTCTTTTCTCTCTCTTTTTTTTTTGAGACAGGGTCTCTCTCTGTCACCCAGGCTGGAGTGC
AGTGGTGCAATCTCAGCTCACTGCAACCTCTGCCTCCCAGGTTCAAGTGATTCTCCTGTCTCAGCTTCCC
AAGTAGCTGGGATTACAGGCGTGCACTACCACACCCGGCTAATTTTTGTATTTTTAGTAGAGACAGGGTT
TCGCCATGTTGGCCAGGATGGGCTTGAACTCTTGACCTCAAGTTATCTGCCCACCTCAGCCTCCCAAAAT
GCTAGGATTACAAGCATGAGCCTCCCAAAGTACTGGGGTTACAGGCATGAGCCACCACTGCCCGACCCAT
GGAAGAGTTTTGAGCAGAGGAGGGACATGATCTGACTTTTAAATGTTATTTATTTATTTTTTAATTTTCT
GAGACAGGGTCTTGCTCTGTCACCCAGGTTGAAGTGCAGTGGTGCGATCTCGTCTCACTGCAGCCTGGAC
CCTTCTGAGCTCAAGTGTTCCTCCCACCTCAGCCACCAGAGTAGCTGGGGCTACAAGCACGCCCCACCAT
GGCCACTAATTTTTGTATTTATTGTAGAGATGGGGTTTTGCCATGTTGCCCAGGCTAGTCTCAAACTCCT
GAGCTCAAGTGAGTTTGCCTCACCCTCCCAAAATGCTGGGATTACAGCTGTGAGCCACCGCGCCCTGCCG
ACATGATCTGACTTAATAGGATTTTTCTGGCTGGTATACTGAGAGTAGATGAAAGAGTGGAAGGAAGACC
AGCAAGGAGGCTACTGCAATAGTCCAGGCATGGGATGCTGGGGGCAGGTGTAGGATACAGGGAAAAGACA
TGGCTCTGCTTCTGGATGTATTTTGAAGGTAGAGCTGGCAGGATTTTCCAGTTTATGGGCACCTAGGGAG
GAAGTGAAGACAAAGAAGAGTGTCAAATATTGAGGTGCAGGGTCAGTTTGAGGACCCACGGAGGAGAAAA
TGACAAAAGGGGGTTGGAGGGAGTGACAACTATGTAGGATGAGAATCGAGTTGGTGACATTGTTGCAGCC
AAGAGGACAAACAGCCACAGGAGGCCAGGGTGCCAGATGCTGCCAAGATGTAATGTAACATGAAGACTGA
ATTTTGACAACATGGCAATCAATGGAATAAGAAAAAAGAAAAGAATTGCATTAGGCTCAGAGAGGTTAAG
CAATCTACCCAAGATCACACAGCAAGGCAGTAAAAGGCCAGAACCCAGGATTCTTGGCTTCCCAAGAGCA
CCTCCCATCCTGGCCCCCCATCAGTGCCTCCCTATCAGGGCTGATGACCTGGTGCCGCCAGCACAGGTGG
GTGGGAGGCCGTCAAGGTCCCTCCTGGTGGGTGGGAAAGAACAGAGCTCACGCTGACCATCCTCCCCTGC
CCTGCCCAGGCTTGGGTGTAGAAGACCCTGTGGGTGAAGTCTCTGTCCATGTTGAGCTGTTCACTCATCC
AGGAACTGGGGAACACAAGGTCACAGTGAAAGGTGAGTGGTCTGAGGTGGGAGGGACTTGCTAGGTTCTA
AAACAGAAACCAAACTCAAACTGGCTTAAGCAACACCCTTTTTTTGTTGTTGTTGTTTAATGGTCCATGA
AACGGAAACGTCCAAAGGTGGCCACCTTCAGGCATGGCTGGATCCAGCAGTGTAAACAGTTTTGTAAGAC
TGAATCCCACTTTCTCCATCTCTCAGCCCTGCTTTCCTTTTCTTGGCTTTTGTTGACTAGAACACAGATT
GGGCTGTTTTTGACATGGCTTAAATAAGACAGAATTAGGCCAGGCACGGTGGCTCATGCCTGTAATCCCA
GCACTTTGGGAGGCCAAAGCTGGCGGATCAGGCCAGGAGTTCAGACCAGCCTGGCCAACATGGTGAAACC
CTGTTTCTACTAAAAATTCAAAAAGTAGCTGGGTGTGGTGGCGTGTGCTTGTAATCCCAGCTACTCAGGA
GGCTGAGGTGGGAGGATCGCTTGAACCCGGGAGGTGGAGTTTGCAGTGAGCTGAGATTGCGCCACTGCAC
TCCAGCCTGGGTGACAGAGTGAGACTCCATGTAAACAAACAAACAAAAATGATAGAATTGGTTTTTCTCT
GGGATAACAGTTTAAGTAGCAACCAGCCTCGAGCTGATATGGAGGCTTCGCAGTGTTGGTGATCTGTGCT
GCTGCCATCTTTTGTTGCATTTTCATCCCTAATGTGTTGTTGTTGTTGTTTGTTTGTTTTAGAGGCCAGA
GTCTCACTCTGTTGCCCAGGTTGGAGCGCTGTGGTGCAATCACAGCACACTGCAGCCTCAAACTCCTGGG
CTCTAGCGATCCTCCTGCCTCAGCTTCCGGAGTCGCTGGGATTGCAGGCATGCAGCATCACACCTGGCTA
CTTTTTAAATTATATTTTTGTAGAGATGGTGTCTCACTATGGTATCCAGGCTGGTCTCAAACTCCTGGCC
TCAAGCAGTCCTCCCACCTGGGCCTCCCAAAGTGCTGGGATTCCAGACGTGAGCCACCATACCTGGACCC
AAATGTGTTTTTCCTCCACTATAGGGTCACAAAGAGCTCAGCCCCCACAAGCATTGCAGACTGCGGCTAG
AGGAAGGAGAAAGTTTGGGGTTCACTCCCTGCCTTTTAAGCCACAGTGGAGAAGTAGTACACGTGATTTT
TTTCTCTCACCTCATTGGCCAAAACATAGTCATATGACCACACTTAGCTTCATGGCAATGCTGGGAAATG
TAGTCTTTATTTCAGACAGGGTTTTTGGCGGCGGGCGAGGGGTGGGGTGTTTTTATTTTTTATTTTTTTT
AGACAGGATCTTGCTTTGTCACACAGGCTGGAGTGCAGTGGCACAATCTCAGCTCACTGCAACTGCCGCC
ACCCAGGCTCAAGCAATCCTCCTGCTTTAGCCCCACAAGTATGTGGGACTACAGGCGCGTGCCACCACGC
CCAGCTGATTTTTGTATTTTTCGTAGAGACAGGGTTTCACCATGTTGCCCAGGCTGGTCTCAAACTCCTG
GACTCAAGTGATCCACCCGCCTTGGCCTCCCGAAGTGGTGGGATTACAAGCGTGAGCCACTGCGCCCAGC
CTTGGGCAGGTTTTGAGAGCCACAAACATCTACTTTTTCCAACTGGGAGAACCTGACCTGTCTTACCCTC
TACTTCCTCCAACAGTGGTGGCTGCCAATGACCTCAAGTGGCAGACTTCTGGCATCTTCCGGCCGTTCAT
CGAGGTCAACATCATTGGGCCCCAGCTCAGCGACAAGAAACGCAAGTTTGCGACCAAATCCAAGAACAAT
AGCTGGGCTCCCAAGTACAATGAGAGCTTCCAGTTGTGAGTTTGGGATGCTTTGAAGAGCATGGGGGCAA
GGGAGGGGGCATCCGCCAGGTGAACCCCAAAGCCAGCTAGGCAGGAGGTATCTGGAATCTGGGCCCGGGA
ATGGGAGGGGCCACATTCTAGGTGGGGGTGCTGGAAAGGGAGACCCCTTAGTAGTGCAGGAATGACATCA
GGGAGGGGACACGAGGATGGAGGTCACCTTTGGGGGCTGGCAGATCCAAGGGGAGGCAATGAGTAGAGTG
GGGTAACTTCATGATGAAGACTTGCAGAGGGGAGGGAAAGTCTGGGCAGGAAGTGCATGGTGGGCAGGGC
CATTGCTGGGGCTGGAGACTCTGTCTGGTGAAGGAATGAGGGTGGCAGTGATGACGCTTATGAGCGGGGG
GCTGAGGGACCTGGGGACAGGACTGAGGTTGCATGGGGGTGGGGACTACTGGAGTCTACATTGAGCCTTG
TCAGGACCGTTGGGGCAGCCCATCTGGAGGAGTCGGCTCTGGGGACAGGAGTGGAGTGTAGATGGATCCC
AGGCTTGGGGGTGGGTGTCACGTGGGGTGGAGGTGGGGTCACATTGGATGGAGGTTCTTGGAGTTGGGAG
TGGGCCTAGGCTGCGGGTATCAGGATAGGGTCTAGAGGATGCTGATGATGTGGACAGCATAAAAAAGTCT
GCTCTGGCCGAGTGTGGTGGCTCACACCTGTCATCCCAGCACTCTGGGACGCCGAGGCAGACAGATCACC
TGAGGTCAGGAGTTCAAGACCAGCCTGGCCAACATGGTGAAACCCCGTCTCTACTAAAAATACAAAAAAA
AAAAAAAAAATTAGCCGGGTGTGGTGGCGCACGCCTGTGATCCCAGCTACTCGGGATGCTGAGGCAGAAG
AATCGCTTGAGTCCGGGAGGCGGAGGGTGCAGTGAGCCAAGATCGAGCCATTGCACTCCAGCCTGGGCAA
CAAAAGCGAAACTCCACCTCAAAAAAAAAAAAAAAGTCTGCTCTGGCCGAGCATGGTGGCTCACGCCTGT
AAGCTCAACACTTTGGGAGGCCAAGGTGGGCAGATAACTTGAGGCCAGGAGTTCGAGACCAGCCTGGCCA
ACATGGTGAAACTCCATCTCTATTAAAAATATAAAATATGGCTGGGTGTGGTGGCTCACACCTGTAATCC
CAGCACTTTGGGAGGCCAAGGCGGGCAGATCACGAGGTCAGGAGATCGAGACCATCCTGGCTAACACGGT
GAAACCCCGTCTCTACTAAAAATACAAAACCTAGCCAGGCGTGGTGGCGGGCGCCTGTAGTCCCAGCTAC
TCGGGAGGCTGAGGCAGGAGAATGGCGTGAACCCGGGAGGCAGAGCTTGCAGTGAGCCGAGATCGCACCA
CTGCACTCCAGCCTGGGCAACAGAGCAAGACTCCGTCTAAAAGAAATATATATTATATAATTATATATAA
AATATGTATTTATATTATATAATTATATATAAAATATGTATTTATATTATATTATATAATATATAATAAA
TATATATCATATAAATATATTTTTATATATTATATATAAATATATATATATTTATATACATATATAATAT
ATATTTATATTATATATAAAATTATATATAGTATATATTATATAGTATAAATAAAATTATATATAGTATA
TATTATATTATATATAAAAGTATATATATTATATATTTCATATATTATATATTGTATATAATATAGTATA
TACAAAAAATATTAGCTGTGTGTTGTGGTGGGCGCCTGTAATCCCAGCTACGCGGGAGGCTGAGACAGGA
GAATCGCTTGAACCCAGGAGGTGGAGGCTGCAATGACCCAAGATGGCGCTACTGCAGTCCAGCCTGGGCT
ACAGAGTGAGACTCAAAAAAATAAAAAATACAGAAATTAGCCGGGTGTGGTGGCAGGCGCTTGTAATCCC
AGCTACTCCAGAGGGTGAGGGGTACGAGAATCGCTTGAACCCAGGAGGCACAGGTTGCAGTGAGCCATGA
TTGTACCACGGCACTCCAGCCTGGGCGACAGAGCAAGACTCTGCCTCAAAAAAAAAAAAAAAAAAAAAGT
CTGTTCACGGGGGTAGATGGATGGTGGTGGTTAGCAAGGGTAGAGTTGGGGTCATCCGGGGTAGATCCTT
GGGTCGAGTGTCAGCCTCACAGAGCACTGGGGCAGGGCAATGTTGTTGGATGTTCTTAGGAGACAGAAGT
GCAGAAGAAAGTAGTCCCAGCTACCTGGGAGGCTGGGGCAGGAAGATCGCTTGAGCTCAGGAGTTTAAGG
CTGCAATGAGCTATGATTGTGCCACTGCACTCCAGCCTGGGTGACCGACATAGCGAGACCCTGTCTTTAA
AAAAACAAAAAACTATGGGCCAGGCGCGGTGGCTGACGCTTGTAATTCCAACACTTTGGGAGGCCAAGGC
AGTGGATCACCTGAGGTTAGGAGTTCGAGACCAGCGTGGCCAACATGGTGAGACCCCCCCCCCCGCCACC
CCCATCTGTACTAAAAACACAAAAAATCAGCCCGGTGTGGTGGTGCATGCCTGTAATCCCAGCTACTAGG
GAGGCTGAGGCAGAAGAATCGTTTGAACCCGGGAGACAGAGGTTGCAGTGAGCCGCGATTGTGCCACTGC
ACTCCAGCCTGGGTGACAGAGGGAGATGCCCACTCAAAAAATAAAAATAAATAAAAATAAATAAAAATGC
ACAGGGGTAAGGGTAAATCAGAGCTGGTTAGCTGGTATTATCTAGGGGGCCTGGGAGCTCTTATGGGACA
AGATTGAAGCCAGTTCCATGTCGTGTCAGCACAGGGCTCTTGGTCAGAAGGTTTTGGGGCGATTGGCCCC
TTTAGGGGATACTCAGGAGGAGAAGCATGGTCTAGTGGGAGGTCAGATGTCAGCCAGGGTGGGCCGGAGT
ACTTCAGGGATAGAGGTACAACCTGGGAGGTGGACAAGAGGGAGAAGCTGGGCGTGACCCTGTAGGAGGG
GCTCTTATCCAGCTGGCGTCAGGGCAGGTCACTGGGACTGCTTAGGGGCAGGAGCACAGGTGAGGGGCAT
GCGGGAGGCGGTGTCACCCATGGTGAGGCACCAGGGCAGGCTCATGTCTGGAGGGCATCTTGGAAGTAAG
AGGGGTGGGCTGGGGGTGTGGGGGGCTGGGGAAAGGGCCACGTCGTGGGTCTAGAGGAGAGCCTGGTTAA
AGAGCTTCTGGGTGGGACATTAGGGGCAGGGCCGTGCGGGCAGCAGTTCAGCCGGCAGAGGGCACAGGCG
CGGGTATCTCAGGGTGGGAGCATGGCCTTGCAGGGCGGTGTCTGAGGGGCGGGAGCTGGATGTAAAACTA
TGCGGTCATTTTTCGGTGGGTGGAAAAATGGGAAGAACAGGCGTGGGCGTGGCCCAGCTCGGGGTCAGCT
GCTGTGGGCGCGGCCAAGCACAGGGTTGTAGGTCAAGTAACGGGGCAGGGGCGGGGCTAGCGTAGCGTGG
GAGGTGACAGGTGTGGGCACCCCGGTGTGGGCGTGGCCCGCAAATGCGGTGGGGACGGGGCCGTGGGCGG
GGCATCACAGTAGGTGTGGCCTGTCTCACGTTCCGCCCCTCCCCACGCAGCACGCTGAGCGCCGACGCGG
GTCCCGAGTGCTATGAGCTGCAGGTGTGCGTCAAGGACTACTGCTTCGCGCGCGAGGACCGCACGGTGGG
GCTGGCCGTGCTGCAGCTGCGTGAGCTGGCCCAGCGCGGGAGCGCCGCCTGCTGGCTGCCGCTCGGCCGC
CGCATCCACATGGACGACACGGGCCTCACGGTGCTGCGAATCCTCTCGCAGCGCAGCAACGACGAGGTGG
CCAAGGAGTTCGTGAAGCTCAAGTCGGACACGCGCTCCGCCGAGGAGGGCGGTGCCGCGCCTGCGCCTTA
GCGCGGGCGGTCGGCCGAGCGGCACTGCGCCTGCGCGGAGGGCGCTGGGCGGGGAGGGACGGGGCTTGCG
CCTTGGTGGGACCTCCCCAGGGGCGGGGCTCGGGGGGCTCCACGCCAAGGGTGGGCTGCGCCTACGCCCT
TGACTCAGCTTTCCCTTTTGGGGAATTAGGAATGGAGGATGCCCCGCCCTCTCGGGAGGCCACGCCCAAG
GGCGCGACGAAGGAAGGAGCCACATCCCCAACTTGAGGCCACGCCCCCAGCACCTAGGGGGCATTTTGAG
CTGGGATGGGGGAAACCTCGTCCCTATGGAGGAGGCCACATCCCGGGGCTCTGGTACCGGGAGGCACCAC
CTCATGTCCCCTGGAAAAGCCATAAGATGGGACCCAGACCCCTGGGACCCCAGACCAATTGCCAAGTATG
GAAATCTCAGCTCCCTCGAGGGGGGGCCCTGGGCAAGGGGTAGGGCTCTCTGGAGCGCCCCTCTAGGTGG
CCTGGGGACTGGAGGGACCAGGATGCTGGTTGGAGGGCCCCGGAATACCGGAGTCCCTTTAGATATTTGT
GCAAAAAATAAATGGGGGGAGGGGGGAGGATGGGATTTCAAAAGCACATGCGCCCTTGGGCGCCCAAACC
CTGGGGGCCGAGGGGACGGCTCTGGTTCCCCACGCTGCCCCTACTTCCCTTTGGGAGTTTGCCTCTCCCT
CTCCCCCAACAAACCCAGTCCTCATATCATAGAGTTCAACACACCCATTTGACAGATGGCAAAACTGAGG
CTTAAAGAGCTGCTTGAGACTTGGCCAAGGTTCCAGGTGCCATACCCTCTGTGCCCCTCCCTTAGGCCTG
TGTGCCCCATGGAAGGGTGGGCTGAGATCGGGATGACCTGACACAGCTCCCTATTGCTGCTAATTCCCCC
TCGGCCTCCTCCAAGGGGTGGGAATTCCAGGCCAAGACCCCTACTTCGCCTTTCCTTCTCCGGCTGCCAA
GCAGGACCTTTGCCCTCAGCCCTTTCTCCTGGGATCTCCATGGGGGATGCCATGAGGGCCTCCCACCACA
AAAGAGAATTTGGGATCCCCTGGTCCCAGGTTTCTCCATCCCTTCTTCCTTTTCCAGAATTTTCCAAATA
GGAAAGAACAGAAGGAGACCAGAAACTCTAGGGGGGAGAAAGAGAATGAGAGAAAGAGAATGAGAGAGAG
AGAAACACAAACACAGTGACACAGTGAGAGCTTAGTCTCCAAGAGCCTATTCATTGATTCAAACACCCAA
GCCACAGGATACCTCAGATGGCCCTCTTGCCAGCTGGAAGCTCTTTCTCCAATGAGCAAAGTTACAGTGA
CCTGGCTGGAGTTACCTGGTGCACATAGGACCTTAGGGGAAAGTTCAGCGTGGACTACACTTGCTCTGGG
ATCTGCTTTTCCACATGTGTGTATGGCACGCCTTTTTCTGCTGGATTGGGAAGGACAAGATTTTGCTGTG
CTAGGGAGAAATGAAAACGGGGTGAGCTGAGTAGCTGGGTTTCTGGAGGATAGAACATCAGATGGGGAGG
CTTTCCGAGGTGAAGAATGAGAGGGAACCACTTACTAGAGAGAAAAGAGCTCCAGGCCTGGGGAACAGCA
CGTGCGAAGGCCAGGAGAGAAGAACTGTTGAAACAACGAGAAGGGTGGCACGGCTGGAGCTGAGCCAGCA
AGGGGGATCGTGAGGAGCCTTGGGGTTGGGGAGATCTGCAGAAGCATCAGACCAGGCAGGGCCTCGTACG
CAGTCCTGAGGAGTTTTACTTTTATTCTAAGACAGTTGGGGAGCTCCAGGAGCTGTTTTAAGTTGGGGAG
AGACTGGATTCCAGCCTGCAAAAGCTGTTTTGTGAAGACTAAAACCAGTGAGGAGAGGTGGAGGTGCTTT
GGGGACACTGAAATGGATTCTTGGAAAGATTCTGAAGGCTGTGTTGAAAAGACACCTATAGCTGTGGGGA
CATGACTATAATCCCAGCATTTGGGGAGACCGAGGCTGGCAGATCACTTAAGGTCAGGAGTTTGAGACCA
GCCTGGCCAACATGGCGAAACCCCATCTCTGCTAAAAATACAAAAATTAGCTGGGTGCAGTGGTGCATGC
CTGTAGTCCCAGCTACTCAGGAGACTGAGGCGGGAGAATTGTTTGAACCCTGGAGGCAGAGGTTGTAGTG
AGTCGTGATCACACAACTGCACTCCAGCCTGGGCAACAGAACAATACTCCATTCCCTCCCCTCTACCCCA
CCAAAAAAAAAAAAAAATCCTGCCCTTAGATGAGCTCTAGGGCTGCTGAGTACAGTTGTCCCAGTTGCAC
AGTGCCCAAGGGTTTGGCATTGCTAAGAAGGCCACGTGCAAATCCTAGATATTGAGTGTTGTATGTTTGT
GACGTTGGTTTCCCGACATGTGAATGGCCCAAGTGTCTGGAAGAAGTGGCGCCACTTTCTAATTTGCTTG
GAGATGTTGCATGTCCCTTAAATTCAGACAGGTGCAGGTAACTGGAGGTTCTGAACCAAAGGTTAAAATG
CAAATTCTCATACAGGGTTGGGAAGTTGTAGCCAGGGATAAGCTTATGTGACTGTTATATGGACTGAGGA
GCAGATGTGAATTTCGAACCATGACATGGCTGAGGGTAGGGGTCGGGTGGATGGATGATTCAGGGTTGTA
ACCCATAGAGCCCAAAGGGGAAGTGATCTGTGACCTGGGGTGAGGGTGATCTGGAAGATTTTTGGATGGC
TGGAAAGAAATGGGGAAGTCGAGCTGCCTGAGAGAGCCAAGTTATTTCCCAAAAGATTCCTTAGGAGTCT
TTCTGTTCAAGACCTCCGTGTGTGTGTGTGTGTGTTTAGGGTTCCCCAGCAATGGCCCAGGCATGTGAAG
GAAACAAGCTTCTTCAGGGAATATTTGTTGAATGAGTTTTCCTGACTCCCAGGCTAGAACTGTTTTTGCA
ATTTCCACCCTCTTTTCTTTCCCCCAGAGAACTCCTATTCGTCCTTCAAAACCCATCACGGAAACCCCTC
TTGGAGAAAACCCTCCTTCCTTCCCCTCAGGACTTTCCCAGCCACCGTCTCTCCTCCAGTCCAGCCTGAT
GCCATGGGACTGGGGGTTTCTCTGTCCAGCTCTGTTTCTCCCAGACTGGGGTCTGAGGACTCTCAGGACC
CCCAACTTTACCTAGCACAGGCTGGGCACAAGTGGGTGACAGGGAGTCTACGCCTAGTGGAATTATGTAT
TGGGGCAGGGTCAGTGTGAGAATACACATCCGCATGCATGTCTGTCCATGTCTGTCCGTACCAACCTTCC
CCTTCCACACGGACCTGGGCACATAGGAGGTGTCTGAGCCTGACACATGGGACAGAGAGTGGACATGGCT
GAGACACGGACAGAGAAAAGACAAGGAGTCCAGGGGGCTGAAAGCCTTTTGAAATCAGGAAGTTCCTGTA
TTGGCAGAACAAAGCCCAGAGAGGAGCAGGGCTTTCCTCAACGCCACCCAGCAAGTGGACACAGAGCCCG
GCCTTGGATGACACCTCCAGGGTTCTGAACCCTGGACCTCGCTTTATGCAAGGAGCTGGCCCCACATTTC
CATGAATCGGGGAAACAGCACAAGAAGGTTGGCCTGTGGCAGGGCAAGGGTTAAAGGGGTGACATTGAGG
GATGCCTCAGAGTCAAAGTCCCCTGACCAAGAGGAATAGAGTAGAAAACACAGAGACAGAGGGTGAGATC
ACGCCCCGATGAGGACGGAGAGAGACAGAGATGGAGAGAGACATAGAGGTGGAAATATACAGAGAAAGAT
AAATGCAGAGACCAAGGCAGGGAGTGTCGGGGGAAGTAAAGAGGGTGTCCTGAAGAAAGAAGGATCTGTT
CACTCTTACCAGTCTGTCCTCGAATGATTTGCATAAAATGAGGAGGTGCCTGTCCACACCCCCAATTCCT
CTCTCAGGCCCCAGAGCCTGAGACCTCACCATGCCCCCATCAGAGATGCAAAAAACTAAACACCCAACTA
GAAATCCTTGGGACCTCTCTCGGCTGGGATCTCAGAGCCTTTCTGTCCCCTACCCCTACCCCATGTGCTG
TCGATTTTGCAGATGGGGACAACCTGGGGCCTCCCGGAACTCTGCCACCCTGGGGAAGTTGGGGGAGGGC
CTTAGTCCCGGATCACAACCCCGTCTGCTCCCCAGAATCCTTTCCTAAGAATCGTTGAGGACCAAAGTTG
TCTTTGCTGACACGTGTTGCTTTTCTCTTTGCCTTTTATTGTTTCAGAGAAAAATCAAGTTGACTGTGTC
AAGTAACACCCCACCCCTTACCCCCGTCCAGCCATAGTGGCTCTCTGGAGACACAGGTCACAGGCGGAGG
GTCCCCTGATCATCCCCAACCACACAGCCAGGGGGACTTGACCCCTGTCCACCCCTGTCTCGTGCTCCCT
CAGACCCCCACAAACCGGCCAAGCAGTCCGGGGAGGCTTCCCCTCCACACAACTCTTAGCATGTGATTGC
AGATGTGAAATCAAAACGTTGTTTGTTTTTTGTTTTGTTTTGATTCTACCCCGTCGGTCCAGTGTCTGCA
CAGACGCCTTCATTTCTCTGTAAATATGTGACTTGGAACAAATGTTTAACACAAACGAGAAGTGGTCATG
AATGCATGGTGTTGAGATGTTTTGCACTATTCTGACTTTTTGGTCTCTGTAAAAATATTTTATTAACAGC
AGACATTAAAAAAAGAAAAACCACACACAGCCTTGGACACGTGGTTGCCTCCTCCTTGCATTCCTTTATC
AGCAAAAAAACAAAAACAAAAACCTCACGTACGATTAGCACCTACTAGGTAGGCGCAGACTCAGTAGAAC
ACACTGCTTGCCCATGAACGCGGCTCATCCGCAACTGGAGTCATCCCCATTTTACAGAGAAGGAAACTGA
AGTACAGAGAGACAAGATCTTGCTCAGATCACATAGCCAGGAAGAGATGGAGCTGGGACTTGAACTCAGA
ACATTTGCAACCATTGCTTTTTTTTTTTTTTTTTGAGATGAAGTCTCTGTTGCCCAGGCTGGAATGCAGT
GGTGCAATCTCAGCTCACTGCAATCTCCACCTCCCAGATTCAAGCGATTCTCCTGCCTTAGCCTCCTGAG
TAGCTGGGATTACAGGCGCCCGCCACCTTGCCTGGCTAATTTTTGTATTTTTAGTAGAGATAGGATTTCA
CCATGTTGGCCAGGCTGGTCGCGAACCCCTGACCTCAGGTGATCCACTCCCTCGGCCTCCCAAAGTGCTG
GGATTACAGGCGTGAGCCACCGCACCCGGCCATCTGTTGCCCTTAAGGAAGTTAAGATACAAACACTAGG
AATACATGCCCTTAATTCAACCCACACCCCCATGCTTGTTTGGTTTTGTTGGGTTTTTTGGGATCTTCTT
GTTTGTTTGTTTTTGAGACAGGGTCTTGCTCTGTCACCCAGGATGGAGTTCAGTGTCACAATCTCAACTC
ACTGCATCCTCAACCTCCCAGGCTCAGGTGATCCTCCCACCTCAGCCTCCTGAGTAGTGTGCACCACCAC
ACCTGGCTAATTTGTGTATTTTTTGTAGTGATAGGATGTCGCCATGTTGCCCAGACTGCTCTCGAACTCC
TCACTTTGGGCAATCCTCTCTCCTCAGCCCTCCAAAGTGTTGGGATTACAGGCGTGAGCCACTGTGCCTG
GCCATGTCTGTTTTAAATGTAGCAGCTGGGGCCAGGCGCAGTGGCTCACGCCTGTAATCCCAGCACTTTA
GGAGGCCGAGGCGGGTGGATCACGAGGTCAGGAGATCGAGACCATCCTGGCTAACACGGTGAAACCCCGT
CTCTACTAAAAGTACCAAAAATTAGCCGGGCACGGTGGCAGGCACCTGTAGTTCCAGCTACTCGGGAGGC
TGAGGCAGGAGAATGGTGTGAACTGGGGAGGCGGAGCTTGCAGTGAGCCAAGATCACGCCACTGCATTCC
AGCCTGGGTGACGGAGCGAGACTCCATCTCAAAAAAAAAAAAAGAAAAAAAAGTAGCAGCTGGGAATCTC
ATTTCATAAACTCAGATGTGTGAGGAATGAAACTCCATTTTCCAACAGCTGAATTTAATTTCTGTTTTGA
GTCTCCATCCCACGAGGAAGCAGAAATGGAATCTCATTCAGCCCAGTTTCCTCGGTAAATTCCGAAATTC
CCCTCCTTATGTCTGGGGTTATTTCTACATTTGAGCGGACAGGACGGGAGCTGCGGGATCCACCACCAGG
AGTGGGTTCAGTGAGTGCTTTTGGTTGCAAGTAACAGACAGCAGGACTAAAAGTGGGGCCAGAAAGTGGC
TTACACCAGGAGTTATCCAGCACGCAGCAATCCCGCTCCTGGGGGACTTTTCTCCTTTGAGAAACTTGCA
CGTGGGGGTGTGGTATCTTCTCAAAGAATAGCATATAGCTGGGAAAAGGGGACAAACCGCAGCTGTCTGC
ATGATGTCTGCAGAGGCAGCCTGCAGGTCCTTGAGTGCCCCTGCTCATTAGCTGTTTCTGTACCCAAGTG
CATAGGCTACGCACTGTGACTGATGTGGATCTATTTGCCCCTCGCTACAAAAGGGTCTCAAGTCTTGTCC
TTCAGTGTGTGGGACCCATCGTGTTACCAATGGGGCTTGGGGACTGAGGCTCAAGACTGCTTGCTGGGAA
ATGTAACCCCTTTGTTCTGGACAGGTAGAAGGAGAGGAGAATGGTCATCCAGGCCTAATGTAAAGTTTCT
GCAACCC
(SEQ ID NO: 5064 (SOURCE NCBI Reference Sequence NG_052872.1)).

In various embodiments, a UNC13A transcript is a pre-mRNA UNC13A transcript. In various embodiments, a UNC13A pre-mRNA transcript comprises a sequence provided as SEQ ID NO: 5065. NCBI Reference Sequence NC_000019.10 Reference GRCh38.p 13 Primary Assembly is SEQ ID NO: 5065.

UNC13A Transcript with a Cryptic Exon

In some embodiments, an UNC13A AON targets a region of an UNC13A transcript comprising a cryptic exon sequence, the UNC13A mRNA transcript comprising the sequence provided as SEQ ID NO: 5206.

(SEQ ID NO: 5206)
TTCTGCCACCATGGGAGAGAAAGTGTCATGGAGAGTGCAAGGGGTCGCCG
GGGGTCAGAGGTCCCAGGGTCTGGGGCAGCTTACATCATCCATCTGCCTG
TTTATTCATTAATTCATTCATCTACTCTTTTATCCATCCACACACCCACC
CATCTAACTACCCCAAATTTCACCCATCCACTCTTCCAACCTTTCAGTAA
TTCAACCACACATCCATCCATCCATCCATTCATCCATCCCATACATTGAT
CCGCAACTTAATCCACCTACCCAATCATTCATTCTTTCATACAACCAACC
ATCCATCCACCCATCAATTTATCCAACCATCCATTTTTCGTCTGTCCACC
AGCCACTCACAACCATCCATCTAAATATTCAGAAATTATGAACCCAATTA
TCAATACTTGCAATAGTTCAACCACACATCCTTCCATTCATCCACCCACC
CATTCATCCATTTGTCCATCTGCCTATCATCCATCCATCCATCCATCCA
TCTACCTATCTACCCATCTGACTATCAACAAATTCACCTATCTACTCAAT
CTTCCTTCTAATAACTCAACCACACTTCCATCCATCCCATCCAATACAAC
TTAATCTGCTCATCCAACATTTCATCTATCCACCCAGTCAATCATCTATC
CAGCAATCTATCTATCCACTCATCAAGTTATCCATCCATCATTCATCTAT
ACTCATCCATCATCTAACAATTACCCCCAAATTCACCCATCCATACATCT
ATACTTTCAATAGTTCAATCATATATCCATCCATCTGTCCATCCATCCAT
CATCCATCTAGCCACGAATCTACCCACCAACTCATCCATCTATCCATCCA
TGTACTCACCCATCTCTCCATCCATCCTTTTATCTACTCATCACTCATTC
ATCTGTTCAATCATTCATTCATTCACCAGCATTTATTCAACAAACTAGTT
CCTGGGGATAAGAGTTCTTTCCAGGAAACCCAGGCAGCTGGAAGAGACAT
ACCCAGACACAAACGGCCCAATCCTGAGTGGTTAGGGCTGGAATAGAAGG
AAGAACCTGATGATGAGTAGTGAGAGTCAACCTGGAGGCCAAGGTGGGCT
TCCCAGAGGAGGTGACCCTGAATCTGGACTTTGATGGATAGGGAGGAGTT
TTCCAGGTAAAGGGAATGGCATGGCGGGCAGTCTCAGAGGGCCAGGAGAG
TGTGGATGGTGTGGCCAGAAAGAGGAAGAGCTGGGCAGGCAGGAATGGTG
AGTGGAAGTGGCATGGGAGGGGCCGAGCAATGACCCTCAC
where underlined = TDP-43 binding site.

In some embodiments, a cryptic exon sequence within the UNC13A mRNA transcript is provided as SEQ ID NO: 5207.

(SEQ ID NO: 5207)
CCATCTCTCCATCCATCCTTTTATCTACTCATCACTCATTCATCTGTTCA
ATCATTCATTCATTCACCAGCATTTATTCAACAAACTAGTTCCTGGGGAT
AAGAGTTCTTTCCAGGAAACCCAGGCAGCTGGAAGAGACATACCCAGACA
CAAACGGCCCAATCCTGAGTGGTTAGGG.

In some embodiments, a cryptic exon sequence within the UNC13A mRNA transcript is provided as SEQ ID NO: 5208.

(SEQ ID NO: 5208)
CCATCTCTCCATCCATCCTTTTATCTACTCATCACTCATTCATCTGTTCA
ATCATTCATTCATTCACCAGCATTTATTCAACAAACTAGTTCCTGGGGAT
AAGAGTTCTTTCCAGGAAACCCAGGCAG.

### UNC13A Oligonucleotides Targeting Regions of the UNC13A Transcript

In various embodiments, UNC13A AON disclosed herein are complementary to specific regions of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065 or SEQ ID NOs: 5206-5208. In some embodiments, a UNC13A AON comprises a sequence that is complementary to a specific region of the UNC13A transcript comprising a sequence that shares at least 90% (e.g., 90%, 91%, 92%, 93%, 94%, 95%, 96%, 97%, 98%, 99%, or 100%) identity to any one of SEQ ID NOs: 5057-5065 or SEQ ID NOs: 5206-5208. In some embodiments, a UNC13A AON comprises a sequence that is at least 85% complementary to a specific region of the UNC13A transcript. In some embodiments, a UNC13A AON comprises a sequence that is at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% complementary to a specific region of the UNC13A transcript. In some embodiments, a UNC13A AON comprises a sequence that is 90 to 99% complementary to a specific region of the UNC13A transcript. In some embodiments, a UNC13A AON comprises a sequence that is 90 to 95% complementary to a specific region of the UNC13A transcript. In some embodiments, a UNC13A AON comprises a sequence that is 95 to 99% complementary to a specific region of the UNC13A transcript.

In some embodiments, the UNC13A AON (e.g., UNC13A AON) has a segment that has, at most, 7 linked nucleosides. In some embodiments, the UNC13A AON has a segment that has, at most, 6, 5, 4, 3, or 2 linked nucleosides. The segments of the UNC13A AON may be separated from other segments of the UNC13A AON through a spacer. The segment of the UNC13A AON is complementary to a specific region of the UNC13A transcript (for example, a UNC13A transcript) comprising a sequence that shares at least 90% (e.g., 90%, 91%, 92%, 93%, 94%, 95%, 96%, 97%, 98%, 99%, or 100%) identity to any one of SEQ ID NOs: 5057-5063 or an UNC13A pre-mRNA transcript transcribed from SEQ ID NO: 5064 or 5065 or SEQ ID NOs: 5206-5208.

### UNC13A Oligonucleotide Variants

In various embodiments, UNC13A AONs include different variants, hereafter referred to as UNC13A AON variants. A UNC13A AON variant may be an oligonucleotide sequence of 5 to 100 nucleobases in length, for example, 10 to 40 nucleobases in length, for example, 14 to 40 nucleobases in length, 10 to 30 nucleobases in length, for example, 14 to 30 nucleobases in length, for example, 16 to 28 nucleobases in length, for example, 19 to 23 nucleobases in length, for example, 21 to 23 nucleobases in length, for example, or 18, 19, 20, 21, 22, 23, 24, 25, 26, 27, 28, 29, or 30 nucleobases in length. A UNC13A AON variant may be an oligonucleotide sequence complementary to a portion of a UNC13A pre-mRNA sequence or a UNC13A gene sequence.

In various embodiments, a UNC13A AON variant represents a modified version of a corresponding UNC13A parent oligonucleotide that includes a nucleobase sequence selected from any one of SEQ ID NOs: 1-1264. In some embodiments, a UNC13A AON variant includes a nucleobase sequence that represents a shortened version of a nucleobase sequence of a UNC13A AON selected from any one of SEQ ID NOs: 1-1264. As one example, if a UNC13A parent oligonucleotide includes a 25mer (e.g., 25 oligonucleotide units in length) a variant (e.g., a UNC13A variant) may include a shorter version (e.g., 15mer, 16mer, 17mer, 18mer, 19mer, 20mer, 21mer, or 23mer) of the 25mer UNC13A parent oligonucleotide. In one embodiment, a nucleobase sequence of a UNC13A AON variant differs from a corresponding nucleobase sequence of a UNC13A parent oligonucleotide in that 1, 2, 3, 4, 5, or 6 oligonucleotide units are removed from one or both of the 3′ and 5′ ends of the nucleobase sequence of the UNC13A parent oligonucleotide. In one embodiment, the corresponding UNC13A AON variant may include a 23mer where two oligonucleotide units were removed from one of the 3′ or 5′ end of a 25mer included in the UNC13A parent oligonucleotide. In one embodiment, the corresponding UNC13A AON variant may include a 23mer where one nucleotide base is removed from each of the 3′ and 5′ ends of the 25mer included in the UNC13A parent oligonucleotide. In one embodiment, the corresponding UNC13A AON variant may include a 21mer where two oligonucleotide units are removed from each of the 3′ and 5′ ends of the 25mer included in the UNC13A parent oligonucleotide. In one embodiment, the corresponding UNC13A AON variant may include a 21mer where four oligonucleotide units are removed from either the 3′ or 5′ end of the 25mer included in the UNC13A parent oligonucleotide. In one embodiment, the corresponding UNC13A AON variant may include a 20mer where two oligonucleotide units are removed from the 3′ end of the 25mer included in the UNC13A parent oligonucleotide and three oligonucleotide units are removed from the 5′ end of the 25mer included in the UNC13A parent oligonucleotide. In one embodiment, the corresponding UNC13A AON variant may include a 20mer where three oligonucleotide units are removed from the 3′ end of the 25mer included in the UNC13A parent oligonucleotide and two oligonucleotide units are removed from the 5′ end of the 25mer included in the UNC13A parent oligonucleotide. In one embodiment, the corresponding UNC13A AON variant may include a 20mer where five oligonucleotide units are removed from either the 3′ or 5′ end of the 25mer included in the UNC13A parent oligonucleotide. In one embodiment, the corresponding UNC13A AON variant may include a 19mer where three oligonucleotide units are removed from each of the 3′ and 5′ ends of the 25mer included in the UNC13A parent oligonucleotide. In one embodiment, the corresponding UNC13A AON variant may include a 19mer where two oligonucleotide units are removed from the 3′ end of the 25mer included in the UNC13A parent oligonucleotide and four oligonucleotide units are removed from the 5′ end of the 25mer included in the UNC13A parent oligonucleotide. In one embodiment, the corresponding UNC13A AON variant may include a 19mer where four oligonucleotide units are removed from the 3′ end of the 25mer included in the UNC13A parent oligonucleotide and two oligonucleotide units are removed from the 5′ end of the 25mer included in the UNC13A parent oligonucleotide. In one embodiment, the corresponding UNC13A AON variant may include a 19mer where six oligonucleotide units are removed from either the 3′ or 5′ end of the 25mer included in the UNC13A parent oligonucleotide.

In one embodiment, the corresponding UNC13A AON variant may include a 18mer where two oligonucleotide units are removed from the 3′ end of the 25mer included in the UNC13A parent oligonucleotide and five oligonucleotide units are removed from the 5′ end of the 25mer included in the UNC13A parent oligonucleotide. In one embodiment, the corresponding UNC13A AON variant may include a 18mer where five oligonucleotide units are removed from the 3′ end of the 25mer included in the UNC13A parent oligonucleotide and two oligonucleotide units are removed from the 5′ end of the 25mer included in the UNC13A parent oligonucleotide. In one embodiment, the corresponding UNC13A AON variant may include a 18mer where one oligonucleotide unit is removed from the 3′ end of the 25mer included in the UNC13A parent oligonucleotide and six oligonucleotide units are removed from the 5′ end of the 25mer included in the UNC13A parent oligonucleotide. In one embodiment, the corresponding UNC13A AON variant may include a 18mer where six oligonucleotide units are removed from the 3′ end of the 25mer included in the UNC13A parent oligonucleotide and one oligonucleotide unit is removed from the 5′ end of the 25mer included in the UNC13A parent oligonucleotide. In one embodiment, the corresponding UNC13A AON variant may include a 18mer where seven oligonucleotide units are removed from either the 3′ or 5′ end of the 25mer included in the UNC13A parent oligonucleotide.

In one embodiment, the corresponding UNC13A AON variant may include a 17mer where two oligonucleotide units are removed from the 3′ end of the 25mer included in the UNC13A parent oligonucleotide and six oligonucleotide units are removed from the 5′ end of the 25mer included in the UNC13A parent oligonucleotide. In one embodiment, the corresponding UNC13A AON variant may include a 17mer where six oligonucleotide units are removed from the 3′ end of the 25mer included in the UNC13A parent oligonucleotide and two oligonucleotide units are removed from the 5′ end of the 25mer included in the UNC13A parent oligonucleotide. In one embodiment, the corresponding UNC13A AON variant may include a 17mer where eight oligonucleotide units are removed from either the 3′ or 5′ end of the 25mer included in the UNC13A parent oligonucleotide.

In one embodiment, the corresponding UNC13A AON variant may include a 16mer where two oligonucleotide units are removed from the 3′ end of the 25mer included in the UNC13A parent oligonucleotide and seven oligonucleotide units are removed from the 5′ end of the 25mer included in the UNC13A parent oligonucleotide. In one embodiment, the corresponding UNC13A AON variant may include a 16mer where seven oligonucleotide units are removed from the 3′ end of the 25mer included in the UNC13A parent oligonucleotide and two oligonucleotide units are removed from the 5′ end of the 25mer included in the UNC13A parent oligonucleotide. In one embodiment, the corresponding UNC13A AON variant may include a 16mer where nine oligonucleotide units are removed from either the 3′ or 5′ end of the 25mer included in the UNC13A parent oligonucleotide.

In one embodiment, the corresponding UNC13A AON variant may include a 15mer where two oligonucleotide units are removed from the 3′ end of the 25mer included in the UNC13A parent oligonucleotide and eight oligonucleotide units are removed from the 5′ end of the 25mer included in the UNC13A parent oligonucleotide. In one embodiment, the corresponding UNC13A AON variant may include a 15mer where eight oligonucleotide units are removed from the 3′ end of the 25mer included in the UNC13A parent oligonucleotide and two oligonucleotide units are removed from the 5′ end of the 25mer included in the UNC13A parent oligonucleotide. In one embodiment, the corresponding UNC13A AON variant may include a 15mer where ten oligonucleotide units are removed from either the 3′ or 5′ end of the 25mer included in the UNC13A parent oligonucleotide. Example sequences of UNC13A AON variants are shown below in Table 2A and Table 2B.

TABLE 2A
Example UNC13A Oligonucleotide Variant Sequences

| | AON Sequence* | | Target Sequence |
|---|---|---|---|
| SEQ ID NO: | (5′ → 3′) | SEQ ID NO: | (5′ → 3′) |
| 2529 | GGAGGGGCCGAGCAATGACCCTC | 3793 | GAGGGTCATTGCTCGGCCCCTCC |
| 2530 | GGGAGGGGCCGAGCAATGACCCT | 3794 | AGGGTCATTGCTCGGCCCCTCCC |
| 2531 | TGGGAGGGGCCGAGCAATGACCC | 3795 | GGGTCATTGCTCGGCCCCTCCCA |
| 2532 | ATGGGAGGGGCCGAGCAATGACC | 3796 | GGTCATTGCTCGGCCCCTCCCAT |
| 2533 | CATGGGAGGGGCCGAGCAATGAC | 3797 | GTCATTGCTCGGCCCCTCCCATG |
| 2534 | GCATGGGAGGGGCCGAGCAATGA | 3798 | TCATTGCTCGGCCCCTCCCATGC |
| 2535 | GGCATGGGAGGGGCCGAGCAATG | 3799 | CATTGCTCGGCCCCTCCCATGCC |
| 2536 | TGGCATGGGAGGGGCCGAGCAAT | 3800 | ATTGCTCGGCCCCTCCCATGCCA |
| 2537 | GTGGCATGGGAGGGGCCGAGCAA | 3801 | TTGCTCGGCCCCTCCCATGCCAC |
| 2538 | AGTGGCATGGGAGGGGCCGAGCA | 3802 | TGCTCGGCCCCTCCCATGCCACT |
| 2539 | AAGTGGCATGGGAGGGGCCGAGC | 3803 | GCTCGGCCCCTCCCATGCCACTT |
| 2540 | GAAGTGGCATGGGAGGGGCCGAG | 3804 | CTCGGCCCCTCCCATGCCACTTC |
| 2541 | GGAAGTGGCATGGGAGGGGCCGA | 3805 | TCGGCCCCTCCCATGCCACTTCC |
| 2542 | TGGAAGTGGCATGGGAGGGGCCG | 3806 | CGGCCCCTCCCATGCCACTTCCA |
| 2543 | GTGGAAGTGGCATGGGAGGGGCC | 3807 | GGCCCCTCCCATGCCACTTCCAC |
| 2544 | AGTGGAAGTGGCATGGGAGGGGC | 3808 | GCCCCTCCCATGCCACTTCCACT |
| 2545 | GAGTGGAAGTGGCATGGGAGGGG | 3809 | CCCCTCCCATGCCACTTCCACTC |
| 2546 | TGAGTGGAAGTGGCATGGGAGGG | 3810 | CCCTCCCATGCCACTTCCACTCA |
| 2547 | GTGAGTGGAAGTGGCATGGGAGG | 3811 | CCTCCCATGCCACTTCCACTCAC |
| 2548 | GGTGAGTGGAAGTGGCATGGGAG | 3812 | CTCCCATGCCACTTCCACTCACC |
| 2549 | TGGTGAGTGGAAGTGGCATGGGA | 3813 | TCCCATGCCACTTCCACTCACCA |
| 2551 | AATGGTGAGTGGAAGTGGCATGG | 3815 | CCATGCCACTTCCACTCACCATT |
| 2552 | GAATGGTGAGTGGAAGTGGCATG | 3816 | CATGCCACTTCCACTCACCATTC |
| 2553 | GGAATGGTGAGTGGAAGTGGCAT | 3817 | ATGCCACTTCCACTCACCATTCC |
| 2554 | AGGAATGGTGAGTGGAAGTGGCA | 3818 | TGCCACTTCCACTCACCATTCCT |
| 2555 | CAGGAATGGTGAGTGGAAGTGGC | 3819 | GCCACTTCCACTCACCATTCCTG |
| 2556 | GCAGGAATGGTGAGTGGAAGTGG | 3820 | CCACTTCCACTCACCATTCCTGC |
| 2557 | GGCAGGAATGGTGAGTGGAAGTG | 3821 | CACTTCCACTCACCATTCCTGCC |
| 2558 | AGGCAGGAATGGTGAGTGGAAGT | 3822 | ACTTCCACTCACCATTCCTGCCT |
| 2559 | CAGGCAGGAATGGTGAGTGGAAG | 3823 | CTTCCACTCACCATTCCTGCCTG |
| 2560 | GCAGGCAGGAATGGTGAGTGGAA | 3824 | TTCCACTCACCATTCCTGCCTGC |
| 2561 | GGCAGGCAGGAATGGTGAGTGGA | 3825 | TCCACTCACCATTCCTGCCTGCC |
| 2562 | GGGCAGGCAGGAATGGTGAGTGG | 3826 | CCACTCACCATTCCTGCCTGCCC |
| 2563 | TGGGCAGGCAGGAATGGTGAGTG | 3827 | CACTCACCATTCCTGCCTGCCCA |
| 2564 | CTGGGCAGGCAGGAATGGTGAGT | 3828 | ACTCACCATTCCTGCCTGCCCAG |
| 2565 | GCTGGGCAGGCAGGAATGGTGAG | 3829 | CTCACCATTCCTGCCTGCCCAGC |
| 2566 | AGCTGGGCAGGCAGGAATGGTGA | 3830 | TCACCATTCCTGCCTGCCCAGCT |
| 2567 | GAGCTGGGCAGGCAGGAATGGTG | 3831 | CACCATTCCTGCCTGCCCAGCTC |
| 2568 | AGAGCTGGGCAGGCAGGAATGGT | 3832 | ACCATTCCTGCCTGCCCAGCTCT |
| 2569 | AAGAGCTGGGCAGGCAGGAATGG | 3833 | CCATTCCTGCCTGCCCAGCTCTT |
| 2570 | GAAGAGCTGGGCAGGCAGGAATG | 3834 | CATTCCTGCCTGCCCAGCTCTTC |
| 2571 | GGAAGAGCTGGGCAGGCAGGAAT | 3835 | ATTCCTGCCTGCCCAGCTCTTCC |
| 2572 | AGGAAGAGCTGGGCAGGCAGGAA | 3836 | TTCCTGCCTGCCCAGCTCTTCCT |
| 2573 | GAGGAAGAGCTGGGCAGGCAGGA | 3837 | TCCTGCCTGCCCAGCTCTTCCTC |
| 2574 | AGAGGAAGAGCTGGGCAGGCAGG | 3838 | CCTGCCTGCCCAGCTCTTCCTCT |
| 2575 | AAGAGGAAGAGCTGGGCAGGCAG | 3839 | CTGCCTGCCCAGCTCTTCCTCTT |
| 2576 | AAAGAGGAAGAGCTGGGCAGGCA | 3840 | TGCCTGCCCAGCTCTTCCTCTTT |
| 2577 | GAAAGAGGAAGAGCTGGGCAGGC | 3841 | GCCTGCCCAGCTCTTCCTCTTTC |
| 2578 | AGAAAGAGGAAGAGCTGGGCAGG | 3842 | CCTGCCCAGCTCTTCCTCTTTCT |
| 2579 | CAGAAAGAGGAAGAGCTGGGCAG | 3843 | CTGCCCAGCTCTTCCTCTTTCTG |
| 2580 | CCAGAAAGAGGAAGAGCTGGGCA | 3844 | TGCCCAGCTCTTCCTCTTTCTGG |
| 2581 | GCCAGAAAGAGGAAGAGCTGGGC | 3845 | GCCCAGCTCTTCCTCTTTCTGGC |
| 2582 | GGCCAGAAAGAGGAAGAGCTGGG | 3846 | CCCAGCTCTTCCTCTTTCTGGCC |
| 2583 | TGGCCAGAAAGAGGAAGAGCTGG | 3847 | CCAGCTCTTCCTCTTTCTGGCCA |
| 2584 | GTGGCCAGAAAGAGGAAGAGCTG | 3848 | CAGCTCTTCCTCTTTCTGGCCAC |
| 2585 | TGTGGCCAGAAAGAGGAAGAGCT | 3849 | AGCTCTTCCTCTTTCTGGCCACA |
| 2586 | GTGTGGCCAGAAAGAGGAAGAGC | 3850 | GCTCTTCCTCTTTCTGGCCACAC |
| 2587 | GGTGTGGCCAGAAAGAGGAAGAG | 3851 | CTCTTCCTCTTTCTGGCCACACC |
| 2588 | TGGTGTGGCCAGAAAGAGGAAGA | 3852 | TCTTCCTCTTTCTGGCCACACCA |
| 2589 | ATGGTGTGGCCAGAAAGAGGAAG | 3853 | CTTCCTCTTTCTGGCCACACCAT |
| 2590 | GATGGTGTGGCCAGAAAGAGGAA | 3854 | TTCCTCTTTCTGGCCACACCATC |
| 2591 | GGATGGTGTGGCCAGAAAGAGGA | 3855 | TCCTCTTTCTGGCCACACCATCC |
| 2592 | TGGATGGTGTGGCCAGAAAGAGG | 3856 | CCTCTTTCTGGCCACACCATCCA |
| 2594 | TGTGGATGGTGTGGCCAGAAAGA | 3858 | TCTTTCTGGCCACACCATCCACA |
| 2595 | GTGTGGATGGTGTGGCCAGAAAG | 3859 | CTTTCTGGCCACACCATCCACAC |
| 2596 | AGTGTGGATGGTGTGGCCAGAAA | 3860 | TTTCTGGCCACACCATCCACACT |
| 2597 | GAGTGTGGATGGTGTGGCCAGAA | 3861 | TTCTGGCCACACCATCCACACTC |
| 2598 | AGAGTGTGGATGGTGTGGCCAGA | 3862 | TCTGGCCACACCATCCACACTCT |
| 2599 | GAGAGTGTGGATGGTGTGGCCAG | 3863 | CTGGCCACACCATCCACACTCTC |
| 2600 | GGAGAGTGTGGATGGTGTGGCCA | 3864 | TGGCCACACCATCCACACTCTCC |
| 2601 | AGGAGAGTGTGGATGGTGTGGCC | 3865 | GGCCACACCATCCACACTCTCCT |
| 2602 | CAGGAGAGTGTGGATGGTGTGGC | 3866 | GCCACACCATCCACACTCTCCTG |
| 2603 | CCAGGAGAGTGTGGATGGTGTGG | 3867 | CCACACCATCCACACTCTCCTGG |
| 2604 | GCCAGGAGAGTGTGGATGGTGTG | 3868 | CACACCATCCACACTCTCCTGGC |
| 2605 | GGCCAGGAGAGTGTGGATGGTGT | 3869 | ACACCATCCACACTCTCCTGGCC |
| 2606 | GGGCCAGGAGAGTGTGGATGGTG | 3870 | CACCATCCACACTCTCCTGGCCC |
| 2607 | AGGGCCAGGAGAGTGTGGATGGT | 3871 | ACCATCCACACTCTCCTGGCCCT |
| 2608 | GAGGGCCAGGAGAGTGTGGATGG | 3872 | CCATCCACACTCTCCTGGCCCTC |
| 2609 | AGAGGGCCAGGAGAGTGTGGATG | 3873 | CATCCACACTCTCCTGGCCCTCT |
| 2610 | CAGAGGGCCAGGAGAGTGTGGAT | 3874 | ATCCACACTCTCCTGGCCCTCTG |
| 2611 | TCAGAGGGCCAGGAGAGTGTGGA | 3875 | TCCACACTCTCCTGGCCCTCTGA |
| 2612 | CTCAGAGGGCCAGGAGAGTGTGG | 3876 | CCACACTCTCCTGGCCCTCTGAG |
| 2613 | TCTCAGAGGGCCAGGAGAGTGTG | 3877 | CACACTCTCCTGGCCCTCTGAGA |
| 2614 | GTCTCAGAGGGCCAGGAGAGTGT | 3878 | ACACTCTCCTGGCCCTCTGAGAC |
| 2615 | AGTCTCAGAGGGCCAGGAGAGTG | 3879 | CACTCTCCTGGCCCTCTGAGACT |
| 2616 | CAGTCTCAGAGGGCCAGGAGAGT | 3880 | ACTCTCCTGGCCCTCTGAGACTG |
| 2617 | GCAGTCTCAGAGGGCCAGGAGAG | 3881 | CTCTCCTGGCCCTCTGAGACTGC |
| 2618 | GGCAGTCTCAGAGGGCCAGGAGA | 3882 | TCTCCTGGCCCTCTGAGACTGCC |
| 2619 | GGGCAGTCTCAGAGGGCCAGGAG | 3883 | CTCCTGGCCCTCTGAGACTGCCC |
| 2620 | CGGGCAGTCTCAGAGGGCCAGGA | 3884 | TCCTGGCCCTCTGAGACTGCCCG |
| 2621 | GCGGGCAGTCTCAGAGGGCCAGG | 3885 | CCTGGCCCTCTGAGACTGCCCGC |
| 2622 | GGCGGGCAGTCTCAGAGGGCCAG | 3886 | CTGGCCCTCTGAGACTGCCCGCC |
| 2623 | TGGCGGGCAGTCTCAGAGGGCCA | 3887 | TGGCCCTCTGAGACTGCCCGCCA |
| 2624 | ATGGCGGGCAGTCTCAGAGGGCC | 3888 | GGCCCTCTGAGACTGCCCGCCAT |
| 2625 | CATGGCGGGCAGTCTCAGAGGGC | 3889 | GCCCTCTGAGACTGCCCGCCATG |
| 2626 | GCATGGCGGGCAGTCTCAGAGGG | 3890 | CCCTCTGAGACTGCCCGCCATGC |
| 2627 | GGCATGGCGGGCAGTCTCAGAGG | 3891 | CCTCTGAGACTGCCCGCCATGCC |
| 2628 | TGGCATGGCGGGCAGTCTCAGAG | 3892 | CTCTGAGACTGCCCGCCATGCCA |
| 2629 | ATGGCATGGCGGGCAGTCTCAGA | 3893 | TCTGAGACTGCCCGCCATGCCAT |
| 2630 | AATGGCATGGCGGGCAGTCTCAG | 3894 | CTGAGACTGCCCGCCATGCCATT |
| 2631 | GAATGGCATGGCGGGCAGTCTCA | 3895 | TGAGACTGCCCGCCATGCCATTC |
| 2632 | GGAATGGCATGGCGGGCAGTCTC | 3896 | GAGACTGCCCGCCATGCCATTCC |
| 2633 | GGGAATGGCATGGCGGGCAGTCT | 3897 | AGACTGCCCGCCATGCCATTCCC |
| 2634 | AGGGAATGGCATGGCGGGCAGTC | 3898 | GACTGCCCGCCATGCCATTCCCT |
| 2635 | AAGGGAATGGCATGGCGGGCAGT | 3899 | ACTGCCCGCCATGCCATTCCCTT |
| 2637 | TAAAGGGAATGGCATGGGGGCA | 3901 | TGCCCGCCATGCCATTCCCTTTA |
| 2638 | GTAAAGGGAATGGCATGGCGGGC | 3902 | GCCCGCCATGCCATTCCCTTTAC |
| 2639 | GGTAAAGGGAATGGCATGGCGGG | 3903 | CCCGCCATGCCATTCCCTTTACC |
| 2640 | AGGTAAAGGGAATGGCATGGCGG | 3904 | CCGCCATGCCATTCCCTTTACCT |
| 2641 | CAGGTAAAGGGAATGGCATGGCG | 3905 | CGCCATGCCATTCCCTTTACCTG |
| 2642 | CCAGGTAAAGGGAATGGCATGGC | 3906 | GCCATGCCATTCCCTTTACCTGG |
| 2643 | TCCAGGTAAAGGGAATGGCATGG | 3907 | CCATGCCATTCCCTTTACCTGGA |
| 2644 | TTCCAGGTAAAGGGAATGGCATG | 3908 | CATGCCATTCCCTTTACCTGGAA |
| 2645 | TTTCCAGGTAAAGGGAATGGCAT | 3909 | ATGCCATTCCCTTTACCTGGAAA |
| 2646 | TTTTCCAGGTAAAGGGAATGGCA | 3910 | TGCCATTCCCTTTACCTGGAAAA |
| 2647 | GTTTTCCAGGTAAAGGGAATGGC | 3911 | GCCATTCCCTTTACCTGGAAAAC |
| 2648 | AGTTTTCCAGGTAAAGGGAATGG | 3912 | CCATTCCCTTTACCTGGAAAACT |
| 2649 | GAGTTTTCCAGGTAAAGGGAATG | 3913 | CATTCCCTTTACCTGGAAAACTC |
| 2650 | GGAGTTTTCCAGGTAAAGGGAAT | 3914 | ATTCCCTTTACCTGGAAAACTCC |
| 2651 | AGGAGTTTTCCAGGTAAAGGGAA | 3915 | TTCCCTTTACCTGGAAAACTCCT |
| 2652 | GAGGAGTTTTCCAGGTAAAGGGA | 3916 | TCCCTTTACCTGGAAAACTCCTC |
| 2653 | GGAGGAGTTTTCCAGGTAAAGGG | 3917 | CCCTTTACCTGGAAAACTCCTCC |
| 2654 | GGGAGGAGTTTTCCAGGTAAAGG | 3918 | CCTTTACCTGGAAAACTCCTCCC |
| 2655 | AGGGAGGAGTTTTCCAGGTAAAG | 3919 | CTTTACCTGGAAAACTCCTCCCT |
| 2656 | TAGGGAGGAGTTTTCCAGGTAAA | 3920 | TTTACCTGGAAAACTCCTCCCTA |
| 2657 | ATAGGGAGGAGTTTTCCAGGTAA | 3921 | TTACCTGGAAAACTCCTCCCTAT |
| 2658 | GATAGGGAGGAGTTTTCCAGGTA | 3922 | TACCTGGAAAACTCCTCCCTATC |
| 2659 | GGATAGGGAGGAGTTTTCCAGGT | 3923 | ACCTGGAAAACTCCTCCCTATCC |
| 2660 | TGGATAGGGAGGAGTTTTCCAGG | 3924 | CCTGGAAAACTCCTCCCTATCCA |
| 2661 | ATGGATAGGGAGGAGTTTTCCAG | 3925 | CTGGAAAACTCCTCCCTATCCAT |
| 2662 | GATGGATAGGGAGGAGTTTTCCA | 3926 | TGGAAAACTCCTCCCTATCCATC |
| 2663 | TGATGGATAGGGAGGAGTTTTCC | 3927 | GGAAAACTCCTCCCTATCCATCA |
| 2664 | TTGATGGATAGGGAGGAGTTTTC | 3928 | GAAAACTCCTCCCTATCCATCAA |
| 2665 | TTTGATGGATAGGGAGGAGTTTT | 3929 | AAAACTCCTCCCTATCCATCAAA |
| 2666 | CTTTGATGGATAGGGAGGAGTTT | 3930 | AAACTCCTCCCTATCCATCAAAG |
| 2667 | ACTTTGATGGATAGGGAGGAGTT | 3931 | AACTCCTCCCTATCCATCAAAGT |
| 2668 | GACTTTGATGGATAGGGAGGAGT | 3932 | ACTCCTCCCTATCCATCAAAGTC |
| 2669 | GGACTTTGATGGATAGGGAGGAG | 3933 | CTCCTCCCTATCCATCAAAGTCC |
| 2670 | TGGACTTTGATGGATAGGGAGGA | 3934 | TCCTCCCTATCCATCAAAGTCCA |
| 2671 | CTGGACTTTGATGGATAGGGAGG | 3935 | CCTCCCTATCCATCAAAGTCCAG |
| 2672 | TCTGGACTTTGATGGATAGGGAG | 3936 | CTCCCTATCCATCAAAGTCCAGA |
| 2673 | ATCTGGACTTTGATGGATAGGGA | 3937 | TCCCTATCCATCAAAGTCCAGAT |
| 2674 | AATCTGGACTTTGATGGATAGGG | 3938 | CCCTATCCATCAAAGTCCAGATT |
| 2675 | GAATCTGGACTTTGATGGATAGG | 3939 | CCTATCCATCAAAGTCCAGATTC |
| 2676 | TGAATCTGGACTTTGATGGATAG | 3940 | CTATCCATCAAAGTCCAGATTCA |
| 2677 | CTGAATCTGGACTTTGATGGATA | 3941 | TATCCATCAAAGTCCAGATTCAG |
| 2678 | CCTGAATCTGGACTTTGATGGAT | 3942 | ATCCATCAAAGTCCAGATTCAGG |
| 2680 | ACCCTGAATCTGGACTTTGATGG | 3944 | CCATCAAAGTCCAGATTCAGGGT |
| 2681 | GACCCTGAATCTGGACTTTGATG | 3945 | CATCAAAGTCCAGATTCAGGGTC |
| 2682 | TGACCCTGAATCTGGACTTTGAT | 3946 | ATCAAAGTCCAGATTCAGGGTCA |
| 2683 | GTGACCCTGAATCTGGACTTTGA | 3947 | TCAAAGTCCAGATTCAGGGTCAC |
| 2684 | GGTGACCCTGAATCTGGACTTTG | 3948 | CAAAGTCCAGATTCAGGGTCACC |
| 2685 | AGGTGACCCTGAATCTGGACTTT | 3949 | AAAGTCCAGATTCAGGGTCACCT |
| 2686 | GAGGTGACCCTGAATCTGGACTT | 3950 | AAGTCCAGATTCAGGGTCACCTC |
| 2687 | GGAGGTGACCCTGAATCTGGACT | 3951 | AGTCCAGATTCAGGGTCACCTCC |
| 2688 | AGGAGGTGACCCTGAATCTGGAC | 3952 | GTCCAGATTCAGGGTCACCTCCT |
| 2689 | GAGGAGGTGACCCTGAATCTGGA | 3953 | TCCAGATTCAGGGTCACCTCCTC |
| 2690 | AGAGGAGGTGACCCTGAATCTGG | 3954 | CCAGATTCAGGGTCACCTCCTCT |
| 2691 | CAGAGGAGGTGACCCTGAATCTG | 3955 | CAGATTCAGGGTCACCTCCTCTG |
| 2692 | CCAGAGGAGGTGACCCTGAATCT | 3956 | AGATTCAGGGTCACCTCCTCTGG |
| 2693 | CCCAGAGGAGGTGACCCTGAATC | 3957 | GATTCAGGGTCACCTCCTCTGGG |
| 2694 | TCCCAGAGGAGGTGACCCTGAAT | 3958 | ATTCAGGGTCACCTCCTCTGGGA |
| 2695 | TTCCCAGAGGAGGTGACCCTGAA | 3959 | TTCAGGGTCACCTCCTCTGGGAA |
| 2696 | CTTCCCAGAGGAGGTGACCCTGA | 3960 | TCAGGGTCACCTCCTCTGGGAAG |
| 2697 | GCTTCCCAGAGGAGGTGACCCTG | 3961 | CAGGGTCACCTCCTCTGGGAAGC |
| 2698 | GGCTTCCCAGAGGAGGTGACCCT | 3962 | AGGGTCACCTCCTCTGGGAAGCC |
| 2699 | GGGCTTCCCAGAGGAGGTGACCC | 3963 | GGGTCACCTCCTCTGGGAAGCCC |
| 2700 | TGGGCTTCCCAGAGGAGGTGACC | 3964 | GGTCACCTCCTCTGGGAAGCCCA |
| 2701 | GTGGGCTTCCCAGAGGAGGTGAC | 3965 | GTCACCTCCTCTGGGAAGCCCAC |
| 2702 | GGTGGGCTTCCCAGAGGAGGTGA | 3966 | TCACCTCCTCTGGGAAGCCCACC |
| 2703 | AGGTGGGCTTCCCAGAGGAGGTG | 3967 | CACCTCCTCTGGGAAGCCCACCT |
| 2704 | AAGGTGGGCTTCCCAGAGGAGGT | 3968 | ACCTCCTCTGGGAAGCCCACCTT |
| 2705 | CAAGGTGGGCTTCCCAGAGGAGG | 3969 | CCTCCTCTGGGAAGCCCACCTTG |
| 2706 | CCAAGGTGGGCTTCCCAGAGGAG | 3970 | CTCCTCTGGGAAGCCCACCTTGG |
| 2707 | GCCAAGGTGGGCTTCCCAGAGGA | 3971 | TCCTCTGGGAAGCCCACCTTGGC |
| 2708 | GGCCAAGGTGGGCTTCCCAGAGG | 3972 | CCTCTGGGAAGCCCACCTTGGCC |
| 2709 | AGGCCAAGGTGGGCTTCCCAGAG | 3973 | CTCTGGGAAGCCCACCTTGGCCT |
| 2710 | GAGGCCAAGGTGGGCTTCCCAGA | 3974 | TCTGGGAAGCCCACCTTGGCCTC |
| 2711 | GGAGGCCAAGGTGGGCTTCCCAG | 3975 | CTGGGAAGCCCACCTTGGCCTCC |
| 2712 | TGGAGGCCAAGGTGGGCTTCCCA | 3976 | TGGGAAGCCCACCTTGGCCTCCA |
| 2713 | CTGGAGGCCAAGGTGGGCTTCCC | 3977 | GGGAAGCCCACCTTGGCCTCCAG |
| 2714 | CCTGGAGGCCAAGGTGGGCTTCC | 3978 | GGAAGCCCACCTTGGCCTCCAGG |
| 2715 | ACCTGGAGGCCAAGGTGGGCTTC | 3979 | GAAGCCCACCTTGGCCTCCAGGT |
| 2716 | AACCTGGAGGCCAAGGTGGGCTT | 3980 | AAGCCCACCTTGGCCTCCAGGTT |
| 2717 | CAACCTGGAGGCCAAGGTGGGCT | 3981 | AGCCCACCTTGGCCTCCAGGTTG |
| 2718 | TCAACCTGGAGGCCAAGGTGGGC | 3982 | GCCCACCTTGGCCTCCAGGTTGA |
| 2719 | GTCAACCTGGAGGCCAAGGTGGG | 3983 | CCCACCTTGGCCTCCAGGTTGAC |
| 2720 | AGTCAACCTGGAGGCCAAGGTGG | 3984 | CCACCTTGGCCTCCAGGTTGACT |
| 2721 | GAGTCAACCTGGAGGCCAAGGTG | 3985 | CACCTTGGCCTCCAGGTTGACTC |
| 2723 | GAGAGTCAACCTGGAGGCCAAGG | 3987 | CCTTGGCCTCCAGGTTGACTCTC |
| 2724 | TGAGAGTCAACCTGGAGGCCAAG | 3988 | CTTGGCCTCCAGGTTGACTCTCA |
| 2725 | GTGAGAGTCAACCTGGAGGCCAA | 3989 | TTGGCCTCCAGGTTGACTCTCAC |
| 2726 | AGTGAGAGTCAACCTGGAGGCCA | 3990 | TGGCCTCCAGGTTGACTCTCACT |
| 2727 | TAGTGAGAGTCAACCTGGAGGCC | 3991 | GGCCTCCAGGTTGACTCTCACTA |
| 2728 | GTAGTGAGAGTCAACCTGGAGGC | 3992 | GCCTCCAGGTTGACTCTCACTAC |
| 2729 | AGTAGTGAGAGTCAACCTGGAGG | 3993 | CCTCCAGGTTGACTCTCACTACT |
| 2730 | GAGTAGTGAGAGTCAACCTGGAG | 3994 | CTCCAGGTTGACTCTCACTACTC |
| 2731 | TGAGTAGTGAGAGTCAACCTGGA | 3995 | TCCAGGTTGACTCTCACTACTCA |
| 2732 | ATGAGTAGTGAGAGTCAACCTGG | 3996 | CCAGGTTGACTCTCACTACTCAT |
| 2733 | GATGAGTAGTGAGAGTCAACCTG | 3997 | CAGGTTGACTCTCACTACTCATC |
| 2734 | TGATGAGTAGTGAGAGTCAACCT | 3998 | AGGTTGACTCTCACTACTCATCA |
| 2735 | ATGATGAGTAGTGAGAGTCAACC | 3999 | GGTTGACTCTCACTACTCATCAT |
| 2736 | GATGATGAGTAGTGAGAGTCAAC | 4000 | GTTGACTCTCACTACTCATCATC |
| 2737 | TGATGATGAGTAGTGAGAGTCAA | 4001 | TTGACTCTCACTACTCATCATCA |
| 2738 | CTGATGATGAGTAGTGAGAGTCA | 4002 | TGACTCTCACTACTCATCATCAG |
| 2739 | CCTGATGATGAGTAGTGAGAGTC | 4003 | GACTCTCACTACTCATCATCAGG |
| 2740 | ACCTGATGATGAGTAGTGAGAGT | 4004 | ACTCTCACTACTCATCATCAGGT |
| 2741 | AACCTGATGATGAGTAGTGAGAG | 4005 | CTCTCACTACTCATCATCAGGTT |
| 2742 | GAACCTGATGATGAGTAGTGAGA | 4006 | TCTCACTACTCATCATCAGGTTC |
| 2743 | AGAACCTGATGATGAGTAGTGAG | 4007 | CTCACTACTCATCATCAGGTTCT |
| 2744 | AAGAACCTGATGATGAGTAGTGA | 4008 | TCACTACTCATCATCAGGTTCTT |
| 2745 | GAAGAACCTGATGATGAGTAGTG | 4009 | CACTACTCATCATCAGGTTCTTC |
| 2746 | GGAAGAACCTGATGATGAGTAGT | 4010 | ACTACTCATCATCAGGTTCTTCC |
| 2747 | AGGAAGAACCTGATGATGAGTAG | 4011 | CTACTCATCATCAGGTTCTTCCT |
| 2748 | AAGGAAGAACCTGATGATGAGTA | 4012 | TACTCATCATCAGGTTCTTCCTT |
| 2749 | GAAGGAAGAACCTGATGATGAGT | 4013 | ACTCATCATCAGGTTCTTCCTTC |
| 2750 | AGAAGGAAGAACCTGATGATGAG | 4014 | CTCATCATCAGGTTCTTCCTTCT |
| 2751 | TAGAAGGAAGAACCTGATGATGA | 4015 | TCATCATCAGGTTCTTCCTTCTA |
| 2752 | ATAGAAGGAAGAACCTGATGATG | 4016 | CATCATCAGGTTCTTCCTTCTAT |
| 2753 | AATAGAAGGAAGAACCTGATGAT | 4017 | ATCATCAGGTTCTTCCTTCTATT |
| 2754 | GAATAGAAGGAAGAACCTGATGA | 4018 | TCATCAGGTTCTTCCTTCTATTC |
| 2755 | GGAATAGAAGGAAGAACCTGATG | 4019 | CATCAGGTTCTTCCTTCTATTCC |
| 2756 | TGGAATAGAAGGAAGAACCTGAT | 4020 | ATCAGGTTCTTCCTTCTATTCCA |
| 2757 | CTGGAATAGAAGGAAGAACCTGA | 4021 | TCAGGTTCTTCCTTCTATTCCAG |
| 2758 | GCTGGAATAGAAGGAAGAACCTG | 4022 | CAGGTTCTTCCTTCTATTCCAGC |
| 2759 | GGCTGGAATAGAAGGAAGAACCT | 4023 | AGGTTCTTCCTTCTATTCCAGCC |
| 2760 | GGGCTGGAATAGAAGGAAGAACC | 4024 | GGTTCTTCCTTCTATTCCAGCCC |
| 2761 | AGGGCTGGAATAGAAGGAAGAAC | 4025 | GTTCTTCCTTCTATTCCAGCCCT |
| 2762 | TAGGGCTGGAATAGAAGGAAGAA | 4026 | TTCTTCCTTCTATTCCAGCCCTA |
| 2763 | TTAGGGCTGGAATAGAAGGAAGA | 4027 | TCTTCCTTCTATTCCAGCCCTAA |
| 2764 | GTTAGGGCTGGAATAGAAGGAAG | 4028 | CTTCCTTCTATTCCAGCCCTAAC |
| 2766 | TGGTTAGGGCTGGAATAGAAGGA | 4030 | TCCTTCTATTCCAGCCCTAACCA |
| 2767 | GTGGTTAGGGCTGGAATAGAAGG | 4031 | CCTTCTATTCCAGCCCTAACCAC |
| 2768 | AGTGGTTAGGGCTGGAATAGAAG | 4032 | CTTCTATTCCAGCCCTAACCACT |
| 2769 | GAGTGGTTAGGGCTGGAATAGAA | 4033 | TTCTATTCCAGCCCTAACCACTC |
| 2770 | TGAGTGGTTAGGGCTGGAATAGA | 4034 | TCTATTCCAGCCCTAACCACTCA |
| 2771 | CTGAGTGGTTAGGGCTGGAATAG | 4035 | CTATTCCAGCCCTAACCACTCAG |
| 2772 | CCTGAGTGGTTAGGGCTGGAATA | 4036 | TATTCCAGCCCTAACCACTCAGG |
| 2773 | TCCTGAGTGGTTAGGGCTGGAAT | 4037 | ATTCCAGCCCTAACCACTCAGGA |
| 2774 | ATCCTGAGTGGTTAGGGCTGGAA | 4038 | TTCCAGCCCTAACCACTCAGGAT |
| 2775 | AATCCTGAGTGGTTAGGGCTGGA | 4039 | TCCAGCCCTAACCACTCAGGATT |
| 2776 | CAATCCTGAGTGGTTAGGGCTGG | 4040 | CCAGCCCTAACCACTCAGGATTG |
| 2777 | CCAATCCTGAGTGGTTAGGGCTG | 4041 | CAGCCCTAACCACTCAGGATTGG |
| 2778 | CCCAATCCTGAGTGGTTAGGGCT | 4042 | AGCCCTAACCACTCAGGATTGGG |
| 2779 | GCCCAATCCTGAGTGGTTAGGGC | 4043 | GCCCTAACCACTCAGGATTGGGC |
| 2780 | GGCCCAATCCTGAGTGGTTAGGG | 4044 | CCCTAACCACTCAGGATTGGGCC |
| 2781 | CGGCCCAATCCTGAGTGGTTAGG | 4045 | CCTAACCACTCAGGATTGGGCCG |
| 2782 | ACGGCCCAATCCTGAGTGGTTAG | 4046 | CTAACCACTCAGGATTGGGCCGT |
| 2783 | AACGGCCCAATCCTGAGTGGTTA | 4047 | TAACCACTCAGGATTGGGCCGTT |
| 2784 | AAACGGCCCAATCCTGAGTGGTT | 4048 | AACCACTCAGGATTGGGCCGTTT |
| 2785 | CAAACGGCCCAATCCTGAGTGGT | 4049 | ACCACTCAGGATTGGGCCGTTTG |
| 2786 | ACAAACGGCCCAATCCTGAGTGG | 4050 | CCACTCAGGATTGGGCCGTTTGT |
| 2787 | CACAAACGGCCCAATCCTGAGTG | 4051 | CACTCAGGATTGGGCCGTTTGTG |
| 2788 | ACACAAACGGCCCAATCCTGAGT | 4052 | ACTCAGGATTGGGCCGTTTGTGT |
| 2789 | GACACAAACGGCCCAATCCTGAG | 4053 | CTCAGGATTGGGCCGTTTGTGTC |
| 2790 | AGACACAAACGGCCCAATCCTGA | 4054 | TCAGGATTGGGCCGTTTGTGTCT |
| 2791 | CAGACACAAACGGCCCAATCCTG | 4055 | CAGGATTGGGCCGTTTGTGTCTG |
| 2792 | CCAGACACAAACGGCCCAATCCT | 4056 | AGGATTGGGCCGTTTGTGTCTGG |
| 2793 | CCCAGACACAAACGGCCCAATCC | 4057 | GGATTGGGCCGTTTGTGTCTGGG |
| 2794 | ACCCAGACACAAACGGCCCAATC | 4058 | GATTGGGCCGTTTGTGTCTGGGT |
| 2795 | TACCCAGACACAAACGGCCCAAT | 4059 | ATTGGGCCGTTTGTGTCTGGGTA |
| 2796 | ATACCCAGACACAAACGGCCCAA | 4060 | TTGGGCCGTTTGTGTCTGGGTAT |
| 2797 | CATACCCAGACACAAACGGCCCA | 4061 | TGGGCCGTTTGTGTCTGGGTATG |
| 2798 | ACATACCCAGACACAAACGGCCC | 4062 | GGGCCGTTTGTGTCTGGGTATGT |
| 2799 | GACATACCCAGACACAAACGGCC | 4063 | GGCCGTTTGTGTCTGGGTATGTC |
| 2800 | AGACATACCCAGACACAAACGGC | 4064 | GCCGTTTGTGTCTGGGTATGTCT |
| 2801 | GAGACATACCCAGACACAAACGG | 4065 | CCGTTTGTGTCTGGGTATGTCTC |
| 2802 | AGAGACATACCCAGACACAAACG | 4066 | CGTTTGTGTCTGGGTATGTCTCT |
| 2803 | AAGAGACATACCCAGACACAAAC | 4067 | GTTTGTGTCTGGGTATGTCTCTT |
| 2804 | GAAGAGACATACCCAGACACAAA | 4068 | TTTGTGTCTGGGTATGTCTCTTC |
| 2805 | GGAAGAGACATACCCAGACACAA | 4069 | TTGTGTCTGGGTATGTCTCTTCC |
| 2806 | TGGAAGAGACATACCCAGACACA | 4070 | TGTGTCTGGGTATGTCTCTTCCA |
| 2807 | CTGGAAGAGACATACCCAGACAC | 4071 | GTGTCTGGGTATGTCTCTTCCAG |
| 2809 | AGCTGGAAGAGACATACCCAGAC | 4073 | GTCTGGGTATGTCTCTTCCAGCT |
| 2810 | CAGCTGGAAGAGACATACCCAGA | 4074 | TCTGGGTATGTCTCTTCCAGCTG |
| 2811 | GCAGCTGGAAGAGACATACCCAG | 4075 | CTGGGTATGTCTCTTCCAGCTGC |
| 2812 | GGCAGCTGGAAGAGACATACCCA | 4076 | TGGGTATGTCTCTTCCAGCTGCC |
| 2813 | AGGCAGCTGGAAGAGACATACCC | 4077 | GGGTATGTCTCTTCCAGCTGCCT |
| 2814 | CAGGCAGCTGGAAGAGACATACC | 4078 | GGTATGTCTCTTCCAGCTGCCTG |
| 2815 | CCAGGCAGCTGGAAGAGACATAC | 4079 | GTATGTCTCTTCCAGCTGCCTGG |
| 2816 | CCCAGGCAGCTGGAAGAGACATA | 4080 | TATGTCTCTTCCAGCTGCCTGGG |
| 2817 | ACCCAGGCAGCTGGAAGAGACAT | 4081 | ATGTCTCTTCCAGCTGCCTGGGT |
| 2818 | AACCCAGGCAGCTGGAAGAGACA | 4082 | TGTCTCTTCCAGCTGCCTGGGTT |
| 2819 | AAACCCAGGCAGCTGGAAGAGAC | 4083 | GTCTCTTCCAGCTGCCTGGGTTT |
| 2820 | GAAACCCAGGCAGCTGGAAGAGA | 4084 | TCTCTTCCAGCTGCCTGGGTTTC |
| 2821 | GGAAACCCAGGCAGCTGGAAGAG | 4085 | CTCTTCCAGCTGCCTGGGTTTCC |
| 2822 | AGGAAACCCAGGCAGCTGGAAGA | 4086 | TCTTCCAGCTGCCTGGGTTTCCT |
| 2823 | CAGGAAACCCAGGCAGCTGGAAG | 4087 | CTTCCAGCTGCCTGGGTTTCCTG |
| 2824 | CCAGGAAACCCAGGCAGCTGGAA | 4088 | TTCCAGCTGCCTGGGTTTCCTGG |
| 2825 | TCCAGGAAACCCAGGCAGCTGGA | 4089 | TCCAGCTGCCTGGGTTTCCTGGA |
| 2826 | TTCCAGGAAACCCAGGCAGCTGG | 4090 | CCAGCTGCCTGGGTTTCCTGGAA |
| 2827 | TTTCCAGGAAACCCAGGCAGCTG | 4091 | CAGCTGCCTGGGTTTCCTGGAAA |
| 2828 | CTTTCCAGGAAACCCAGGCAGCT | 4092 | AGCTGCCTGGGTTTCCTGGAAAG |
| 2829 | TCTTTCCAGGAAACCCAGGCAGC | 4093 | GCTGCCTGGGTTTCCTGGAAAGA |
| 2830 | TTCTTTCCAGGAAACCCAGGCAG | 4094 | CTGCCTGGGTTTCCTGGAAAGAA |
| 2831 | GTTCTTTCCAGGAAACCCAGGCA | 4095 | TGCCTGGGTTTCCTGGAAAGAAC |
| 2832 | AGTTCTTTCCAGGAAACCCAGGC | 4096 | GCCTGGGTTTCCTGGAAAGAACT |
| 2833 | GAGTTCTTTCCAGGAAACCCAGG | 4097 | CCTGGGTTTCCTGGAAAGAACTC |
| 2834 | AGAGTTCTTTCCAGGAAACCCAG | 4098 | CTGGGTTTCCTGGAAAGAACTCT |
| 2835 | AAGAGTTCTTTCCAGGAAACCCA | 4099 | TGGGTTTCCTGGAAAGAACTCTT |
| 2836 | TAAGAGTTCTTTCCAGGAAACCC | 4100 | GGGTTTCCTGGAAAGAACTCTTA |
| 2837 | ATAAGAGTTCTTTCCAGGAAACC | 4101 | GGTTTCCTGGAAAGAACTCTTAT |
| 2838 | GATAAGAGTTCTTTCCAGGAAAC | 4102 | GTTTCCTGGAAAGAACTCTTATC |
| 2839 | GGATAAGAGTTCTTTCCAGGAAA | 4103 | TTTCCTGGAAAGAACTCTTATCC |
| 2840 | GGGATAAGAGTTCTTTCCAGGAA | 4104 | TTCCTGGAAAGAACTCTTATCCC |
| 2841 | GGGGATAAGAGTTCTTTCCAGGA | 4105 | TCCTGGAAAGAACTCTTATCCCC |
| 2842 | TGGGGATAAGAGTTCTTTCCAGG | 4106 | CCTGGAAAGAACTCTTATCCCCA |
| 2843 | CTGGGGATAAGAGTTCTTTCCAG | 4107 | CTGGAAAGAACTCTTATCCCCAG |
| 2844 | CCTGGGGATAAGAGTTCTTTCCA | 4108 | TGGAAAGAACTCTTATCCCCAGG |
| 2845 | TCCTGGGGATAAGAGTTCTTTCC | 4109 | GGAAAGAACTCTTATCCCCAGGA |
| 2846 | TTCCTGGGGATAAGAGTTCTTTC | 4110 | GAAAGAACTCTTATCCCCAGGAA |
| 2847 | GTTCCTGGGGATAAGAGTTCTTT | 4111 | AAAGAACTCTTATCCCCAGGAAC |
| 2848 | AGTTCCTGGGGATAAGAGTTCTT | 4112 | AAGAACTCTTATCCCCAGGAACT |
| 2849 | TAGTTCCTGGGGATAAGAGTTCT | 4113 | AGAACTCTTATCCCCAGGAACTA |
| 2850 | CTAGTTCCTGGGGATAAGAGTTC | 4114 | GAACTCTTATCCCCAGGAACTAG |
| 2852 | AACTAGTTCCTGGGGATAAGAGT | 4116 | ACTCTTATCCCCAGGAACTAGTT |
| 2853 | AAACTAGTTCCTGGGGATAAGAG | 4117 | CTCTTATCCCCAGGAACTAGTTT |
| 2854 | CAAACTAGTTCCTGGGGATAAGA | 4118 | TCTTATCCCCAGGAACTAGTTTG |
| 2855 | ACAAACTAGTTCCTGGGGATAAG | 4119 | CTTATCCCCAGGAACTAGTTTGT |
| 2856 | AACAAACTAGTTCCTGGGGATAA | 4120 | TTATCCCCAGGAACTAGTTTGTT |
| 2857 | CAACAAACTAGTTCCTGGGGATA | 4121 | TATCCCCAGGAACTAGTTTGTTG |
| 2858 | TCAACAAACTAGTTCCTGGGGAT | 4122 | ATCCCCAGGAACTAGTTTGTTGA |
| 2859 | TTCAACAAACTAGTTCCTGGGGA | 4123 | TCCCCAGGAACTAGTTTGTTGAA |
| 2860 | ATTCAACAAACTAGTTCCTGGGG | 4124 | CCCCAGGAACTAGTTTGTTGAAT |
| 2861 | TATTCAACAAACTAGTTCCTGGG | 4125 | CCCAGGAACTAGTTTGTTGAATA |
| 2862 | TTATTCAACAAACTAGTTCCTGG | 4126 | CCAGGAACTAGTTTGTTGAATAA |
| 2863 | TTTATTCAACAAACTAGTTCCTG | 4127 | CAGGAACTAGTTTGTTGAATAAA |
| 2864 | ATTTATTCAACAAACTAGTTCCT | 4128 | AGGAACTAGTTTGTTGAATAAAT |
| 2865 | CATTTATTCAACAAACTAGTTCC | 4129 | GGAACTAGTTTGTTGAATAAATG |
| 2866 | GCATTTATTCAACAAACTAGTTC | 4130 | GAACTAGTTTGTTGAATAAATGC |
| 2867 | AGCATTTATTCAACAAACTAGTT | 4131 | AACTAGTTTGTTGAATAAATGCT |
| 2868 | CAGCATTTATTCAACAAACTAGT | 4132 | ACTAGTTTGTTGAATAAATGCTG |
| 2869 | CCAGCATTTATTCAACAAACTAG | 4133 | CTAGTTTGTTGAATAAATGCTGG |
| 2870 | ACCAGCATTTATTCAACAAACTA | 4134 | TAGTTTGTTGAATAAATGCTGGT |
| 2871 | CACCAGCATTTATTCAACAAACT | 4135 | AGTTTGTTGAATAAATGCTGGTG |
| 2872 | TCACCAGCATTTATTCAACAAAC | 4136 | GTTTGTTGAATAAATGCTGGTGA |
| 2873 | TTCACCAGCATTTATTCAACAAA | 4137 | TTTGTTGAATAAATGCTGGTGAA |
| 2874 | ATTCACCAGCATTTATTCAACAA | 4138 | TTGTTGAATAAATGCTGGTGAAT |
| 2875 | CATTCACCAGCATTTATTCAACA | 4139 | TGTTGAATAAATGCTGGTGAATG |
| 2876 | TCATTCACCAGCATTTATTCAAC | 4140 | GTTGAATAAATGCTGGTGAATGA |
| 2877 | TTCATTCACCAGCATTTATTCAA | 4141 | TTGAATAAATGCTGGTGAATGAA |
| 2878 | ATTCATTCACCAGCATTTATTCA | 4142 | TGAATAAATGCTGGTGAATGAAT |
| 2879 | CATTCATTCACCAGCATTTATTC | 4143 | GAATAAATGCTGGTGAATGAATG |
| 2880 | TCATTCATTCACCAGCATTTATT | 4144 | AATAAATGCTGGTGAATGAATGA |
| 2881 | TTCATTCATTCACCAGCATTTAT | 4145 | ATAAATGCTGGTGAATGAATGAA |
| 2882 | ATTCATTCATTCACCAGCATTTA | 4146 | TAAATGCTGGTGAATGAATGAAT |
| 2883 | CATTCATTCATTCACCAGCATTT | 4147 | AAATGCTGGTGAATGAATGAATG |
| 2884 | TCATTCATTCATTCACCAGCATT | 4148 | AATGCTGGTGAATGAATGAATGA |
| 2885 | ATCATTCATTCATTCACCAGCAT | 4149 | ATGCTGGTGAATGAATGAATGAT |
| 2886 | AATCATTCATTCATTCACCAGCA | 4150 | TGCTGGTGAATGAATGAATGATT |
| 2887 | CAATCATTCATTCATTCACCAGC | 4151 | GCTGGTGAATGAATGAATGATTG |
| 2888 | TCAATCATTCATTCATTCACCAG | 4152 | CTGGTGAATGAATGAATGATTGA |
| 2889 | TTCAATCATTCATTCATTCACCA | 4153 | TGGTGAATGAATGAATGATTGAA |
| 2890 | GTTCAATCATTCATTCATTCACC | 4154 | GGTGAATGAATGAATGATTGAAC |
| 2891 | TGTTCAATCATTCATTCATTCAC | 4155 | GTGAATGAATGAATGATTGAACA |
| 2892 | CTGTTCAATCATTCATTCATTCA | 4156 | TGAATGAATGAATGATTGAACAG |
| 2893 | TCTGTTCAATCATTCATTCATTC | 4157 | GAATGAATGAATGATTGAACAGA |
| 2895 | CATCTGTTCAATCATTCATTCAT | 4159 | ATGAATGAATGATTGAACAGATG |
| 2896 | TCATCTGTTCAATCATTCATTCA | 4160 | TGAATGAATGATTGAACAGATGA |
| 2897 | TTCATCTGTTCAATCATTCATTC | 4161 | GAATGAATGATTGAACAGATGAA |
| 2898 | ATTCATCTGTTCAATCATTCATT | 4162 | AATGAATGATTGAACAGATGAAT |
| 2899 | CATTCATCTGTTCAATCATTCAT | 4163 | ATGAATGATTGAACAGATGAATG |
| 2900 | TCATTCATCTGTTCAATCATTCA | 4164 | TGAATGATTGAACAGATGAATGA |
| 2901 | CTCATTCATCTGTTCAATCATTC | 4165 | GAATGATTGAACAGATGAATGAG |
| 2902 | ACTCATTCATCTGTTCAATCATT | 4166 | AATGATTGAACAGATGAATGAGT |
| 2903 | CACTCATTCATCTGTTCAATCAT | 4167 | ATGATTGAACAGATGAATGAGTG |
| 2904 | TCACTCATTCATCTGTTCAATCA | 4168 | TGATTGAACAGATGAATGAGTGA |
| 2905 | ATCACTCATTCATCTGTTCAATC | 4169 | GATTGAACAGATGAATGAGTGAT |
| 2906 | CATCACTCATTCATCTGTTCAAT | 4170 | ATTGAACAGATGAATGAGTGATG |
| 2907 | TCATCACTCATTCATCTGTTCAA | 4171 | TTGAACAGATGAATGAGTGATGA |
| 2908 | CTCATCACTCATTCATCTGTTCA | 4172 | TGAACAGATGAATGAGTGATGAG |
| 2909 | ACTCATCACTCATTCATCTGTTC | 4173 | GAACAGATGAATGAGTGATGAGT |
| 2910 | TACTCATCACTCATTCATCTGTT | 4174 | AACAGATGAATGAGTGATGAGTA |
| 2911 | CTACTCATCACTCATTCATCTGT | 4175 | ACAGATGAATGAGTGATGAGTAG |
| 2912 | TCTACTCATCACTCATTCATCTG | 4176 | CAGATGAATGAGTGATGAGTAGA |
| 2913 | ATCTACTCATCACTCATTCATCT | 4177 | AGATGAATGAGTGATGAGTAGAT |
| 2914 | TATCTACTCATCACTCATTCATC | 4178 | GATGAATGAGTGATGAGTAGATA |
| 2915 | TTATCTACTCATCACTCATTCAT | 4179 | ATGAATGAGTGATGAGTAGATAA |
| 2916 | TTTATCTACTCATCACTCATTCA | 4180 | TGAATGAGTGATGAGTAGATAAA |
| 2917 | TTTTATCTACTCATCACTCATTC | 4181 | GAATGAGTGATGAGTAGATAAAA |
| 2918 | CTTTTATCTACTCATCACTCATT | 4182 | AATGAGTGATGAGTAGATAAAAG |
| 2919 | CCTTTTATCTACTCATCACTCAT | 4183 | ATGAGTGATGAGTAGATAAAAGG |
| 2920 | TCCTTTTATCTACTCATCACTCA | 4184 | TGAGTGATGAGTAGATAAAAGGA |
| 2921 | ATCCTTTTATCTACTCATCACTC | 4185 | GAGTGATGAGTAGATAAAAGGAT |
| 2922 | CATCCTTTTATCTACTCATCACT | 4186 | AGTGATGAGTAGATAAAAGGATG |
| 2923 | CCATCCTTTTATCTACTCATCAC | 4187 | GTGATGAGTAGATAAAAGGATGG |
| 2924 | TCCATCCTTTTATCTACTCATCA | 4188 | TGATGAGTAGATAAAAGGATGGA |
| 2925 | ATCCATCCTTTTATCTACTCATC | 4189 | GATGAGTAGATAAAAGGATGGAT |
| 2926 | CATCCATCCTTTTATCTACTCAT | 4190 | ATGAGTAGATAAAAGGATGGATG |
| 2927 | CCATCCATCCTTTTATCTACTCA | 4191 | TGAGTAGATAAAAGGATGGATGG |
| 2928 | TCCATCCATCCTTTTATCTACTC | 4192 | GAGTAGATAAAAGGATGGATGGA |
| 2929 | CTCCATCCATCCTTTTATCTACT | 4193 | AGTAGATAAAAGGATGGATGGAG |
| 2930 | TCTCCATCCATCCTTTTATCTAC | 4194 | GTAGATAAAAGGATGGATGGAGA |
| 2931 | CTCTCCATCCATCCTTTTATCTA | 4195 | TAGATAAAAGGATGGATGGAGAG |
| 2932 | TCTCTCCATCCATCCTTTTATCT | 4196 | AGATAAAAGGATGGATGGAGAGA |
| 2933 | ATCTCTCCATCCATCCTTTTATC | 4197 | GATAAAAGGATGGATGGAGAGAT |
| 2934 | CATCTCTCCATCCATCCTTTTAT | 4198 | ATAAAAGGATGGATGGAGAGATG |
| 2935 | CCATCTCTCCATCCATCCTTTTA | 4199 | TAAAAGGATGGATGGAGAGATGG |
| 2936 | CCCATCTCTCCATCCATCCTTTT | 4200 | AAAAGGATGGATGGAGAGATGGG |
| 2938 | CACCCATCTCTCCATCCATCCTT | 4202 | AAGGATGGATGGAGAGATGGGTG |
| 2939 | TCACCCATCTCTCCATCCATCCT | 4203 | AGGATGGATGGAGAGATGGGTGA |
| 2940 | CTCACCCATCTCTCCATCCATCC | 4204 | GGATGGATGGAGAGATGGGTGAG |
| 2941 | ACTCACCCATCTCTCCATCCATC | 4205 | GATGGATGGAGAGATGGGTGAGT |
| 2942 | TACTCACCCATCTCTCCATCCAT | 4206 | ATGGATGGAGAGATGGGTGAGTA |
| 2943 | GTACTCACCCATCTCTCCATCCA | 4207 | TGGATGGAGAGATGGGTGAGTAC |
| 2944 | TGTACTCACCCATCTCTCCATCC | 4208 | GGATGGAGAGATGGGTGAGTACA |
| 2945 | ATGTACTCACCCATCTCTCCATC | 4209 | GATGGAGAGATGGGTGAGTACAT |
| 2946 | CATGTACTCACCCATCTCTCCAT | 4210 | ATGGAGAGATGGGTGAGTACATG |
| 2947 | CCATGTACTCACCCATCTCTCCA | 4211 | TGGAGAGATGGGTGAGTACATGG |
| 2948 | TCCATGTACTCACCCATCTCTCC | 4212 | GGAGAGATGGGTGAGTACATGGA |
| 2949 | ATCCATGTACTCACCCATCTCTC | 4213 | GAGAGATGGGTGAGTACATGGAT |
| 2950 | CATCCATGTACTCACCCATCTCT | 4214 | AGAGATGGGTGAGTACATGGATG |
| 2951 | CCATCCATGTACTCACCCATCTC | 4215 | GAGATGGGTGAGTACATGGATGG |
| 2952 | TCCATCCATGTACTCACCCATCT | 4216 | AGATGGGTGAGTACATGGATGGA |
| 2953 | ATCCATCCATGTACTCACCCATC | 4217 | GATGGGTGAGTACATGGATGGAT |
| 2954 | TATCCATCCATGTACTCACCCAT | 4218 | ATGGGTGAGTACATGGATGGATA |
| 2955 | CTATCCATCCATGTACTCACCCA | 4219 | TGGGTGAGTACATGGATGGATAG |
| 2956 | TCTATCCATCCATGTACTCACCC | 4220 | GGGTGAGTACATGGATGGATAGA |
| 2957 | ATCTATCCATCCATGTACTCACC | 4221 | GGTGAGTACATGGATGGATAGAT |
| 2958 | CATCTATCCATCCATGTACTCAC | 4222 | GTGAGTACATGGATGGATAGATG |
| 2959 | CCATCTATCCATCCATGTACTCA | 4223 | TGAGTACATGGATGGATAGATGG |
| 2960 | TCCATCTATCCATCCATGTACTC | 4224 | GAGTACATGGATGGATAGATGGA |
| 2961 | ATCCATCTATCCATCCATGTACT | 4225 | AGTACATGGATGGATAGATGGAT |
| 2962 | CATCCATCTATCCATCCATGTAC | 4226 | GTACATGGATGGATAGATGGATG |
| 2963 | TCATCCATCTATCCATCCATGTA | 4227 | TACATGGATGGATAGATGGATGA |
| 2964 | CTCATCCATCTATCCATCCATGT | 4228 | ACATGGATGGATAGATGGATGAG |
| 2965 | ACTCATCCATCTATCCATCCATG | 4229 | CATGGATGGATAGATGGATGAGT |
| 2966 | AACTCATCCATCTATCCATCCAT | 4230 | ATGGATGGATAGATGGATGAGTT |
| 2967 | CAACTCATCCATCTATCCATCCA | 4231 | TGGATGGATAGATGGATGAGTTG |
| 2968 | CCAACTCATCCATCTATCCATCC | 4232 | GGATGGATAGATGGATGAGTTGG |
| 2969 | ACCAACTCATCCATCTATCCATC | 4233 | GATGGATAGATGGATGAGTTGGT |
| 2970 | CACCAACTCATCCATCTATCCAT | 4234 | ATGGATAGATGGATGAGTTGGTG |
| 2971 | CCACCAACTCATCCATCTATCCA | 4235 | TGGATAGATGGATGAGTTGGTGG |
| 2972 | CCCACCAACTCATCCATCTATCC | 4236 | GGATAGATGGATGAGTTGGTGGG |
| 2973 | ACCCACCAACTCATCCATCTATC | 4237 | GATAGATGGATGAGTTGGTGGGT |
| 2974 | TACCCACCAACTCATCCATCTAT | 4238 | ATAGATGGATGAGTTGGTGGGTA |
| 2975 | CTACCCACCAACTCATCCATCTA | 4239 | TAGATGGATGAGTTGGTGGGTAG |
| 2976 | TCTACCCACCAACTCATCCATCT | 4240 | AGATGGATGAGTTGGTGGGTAGA |
| 2977 | ATCTACCCACCAACTCATCCATC | 4241 | GATGGATGAGTTGGTGGGTAGAT |
| 2978 | AATCTACCCACCAACTCATCCAT | 4242 | ATGGATGAGTTGGTGGGTAGATT |
| 2979 | GAATCTACCCACCAACTCATCCA | 4243 | TGGATGAGTTGGTGGGTAGATTC |
| 2981 | ACGAATCTACCCACCAACTCATC | 4245 | GATGAGTTGGTGGGTAGATTCGT |
| 2982 | CACGAATCTACCCACCAACTCAT | 4246 | ATGAGTTGGTGGGTAGATTCGTG |
| 2983 | CCACGAATCTACCCACCAACTCA | 4247 | TGAGTTGGTGGGTAGATTCGTGG |
| 2984 | GCCACGAATCTACCCACCAACTC | 4248 | GAGTTGGTGGGTAGATTCGTGGC |
| 2985 | AGCCACGAATCTACCCACCAACT | 4249 | AGTTGGTGGGTAGATTCGTGGCT |
| 2986 | TAGCCACGAATCTACCCACCAAC | 4250 | GTTGGTGGGTAGATTCGTGGCTA |
| 2987 | CTAGCCACGAATCTACCCACCAA | 4251 | TTGGTGGGTAGATTCGTGGCTAG |
| 2988 | TCTAGCCACGAATCTACCCACCA | 4252 | TGGTGGGTAGATTCGTGGCTAGA |
| 2989 | ATCTAGCCACGAATCTACCCACC | 4253 | GGTGGGTAGATTCGTGGCTAGAT |
| 2990 | CATCTAGCCACGAATCTACCCAC | 4254 | GTGGGTAGATTCGTGGCTAGATG |
| 2991 | CCATCTAGCCACGAATCTACCCA | 4255 | TGGGTAGATTCGTGGCTAGATGG |
| 2992 | TCCATCTAGCCACGAATCTACCC | 4256 | GGGTAGATTCGTGGCTAGATGGA |
| 2993 | ATCCATCTAGCCACGAATCTACC | 4257 | GGTAGATTCGTGGCTAGATGGAT |
| 2994 | CATCCATCTAGCCACGAATCTAC | 4258 | GTAGATTCGTGGCTAGATGGATG |
| 2995 | TCATCCATCTAGCCACGAATCTA | 4259 | TAGATTCGTGGCTAGATGGATGA |
| 2996 | ATCATCCATCTAGCCACGAATCT | 4260 | AGATTCGTGGCTAGATGGATGAT |
| 2997 | CATCATCCATCTAGCCACGAATC | 4261 | GATTCGTGGCTAGATGGATGATG |
| 2998 | CCATCATCCATCTAGCCACGAAT | 4262 | ATTCGTGGCTAGATGGATGATGG |
| 2999 | TCCATCATCCATCTAGCCACGAA | 4263 | TTCGTGGCTAGATGGATGATGGA |
| 3000 | ATCCATCATCCATCTAGCCACGA | 4264 | TCGTGGCTAGATGGATGATGGAT |
| 3001 | CATCCATCATCCATCTAGCCACG | 4265 | CGTGGCTAGATGGATGATGGATG |
| 3002 | CCATCCATCATCCATCTAGCCAC | 4266 | GTGGCTAGATGGATGATGGATGG |
| 3003 | TCCATCCATCATCCATCTAGCCA | 4267 | TGGCTAGATGGATGATGGATGGA |
| 3004 | ATCCATCCATCATCCATCTAGCC | 4268 | GGCTAGATGGATGATGGATGGAT |
| 3005 | CATCCATCCATCATCCATCTAGC | 4269 | GCTAGATGGATGATGGATGGATG |
| 3006 | CCATCCATCCATCATCCATCTAG | 4270 | CTAGATGGATGATGGATGGATGG |
| 3007 | TCCATCCATCCATCATCCATCTA | 4271 | TAGATGGATGATGGATGGATGGA |
| 3008 | GTCCATCCATCCATCATCCATCT | 4272 | AGATGGATGATGGATGGATGGAC |
| 3009 | TGTCCATCCATCCATCATCCATC | 4273 | GATGGATGATGGATGGATGGACA |
| 3010 | CTGTCCATCCATCCATCATCCAT | 4274 | ATGGATGATGGATGGATGGACAG |
| 3011 | TCTGTCCATCCATCCATCATCCA | 4275 | TGGATGATGGATGGATGGACAGA |
| 3012 | ATCTGTCCATCCATCCATCATCC | 4276 | GGATGATGGATGGATGGACAGAT |
| 3013 | CATCTGTCCATCCATCCATCATC | 4277 | GATGATGGATGGATGGACAGATG |
| 3014 | CCATCTGTCCATCCATCCATCAT | 4278 | ATGATGGATGGATGGACAGATGG |
| 3015 | TCCATCTGTCCATCCATCCATCA | 4279 | TGATGGATGGATGGACAGATGGA |
| 3016 | ATCCATCTGTCCATCCATCCATC | 4280 | GATGGATGGATGGACAGATGGAT |
| 3017 | CATCCATCTGTCCATCCATCCAT | 4281 | ATGGATGGATGGACAGATGGATG |
| 3018 | CCATCCATCTGTCCATCCATCCA | 4282 | TGGATGGATGGACAGATGGATGG |
| 3019 | TCCATCCATCTGTCCATCCATCC | 4283 | GGATGGATGGACAGATGGATGGA |
| 3020 | ATCCATCCATCTGTCCATCCATC | 4284 | GATGGATGGACAGATGGATGGAT |
| 3021 | TATCCATCCATCTGTCCATCCAT | 4285 | ATGGATGGACAGATGGATGGATA |
| 3022 | ATATCCATCCATCTGTCCATCCA | 4286 | TGGATGGACAGATGGATGGATAT |
| 3024 | ATATATCCATCCATCTGTCCATC | 4288 | GATGGACAGATGGATGGATATAT |
| 3025 | CATATATCCATCCATCTGTCCAT | 4289 | ATGGACAGATGGATGGATATATG |
| 3026 | TCATATATCCATCCATCTGTCCA | 4290 | TGGACAGATGGATGGATATATGA |
| 3027 | ATCATATATCCATCCATCTGTCC | 4291 | GGACAGATGGATGGATATATGAT |
| 3028 | AATCATATATCCATCCATCTGTC | 4292 | GACAGATGGATGGATATATGATT |
| 3029 | CAATCATATATCCATCCATCTGT | 4293 | ACAGATGGATGGATATATGATTG |
| 3030 | TCAATCATATATCCATCCATCTG | 4294 | CAGATGGATGGATATATGATTGA |
| 3031 | TTCAATCATATATCCATCCATCT | 4295 | AGATGGATGGATATATGATTGAA |
| 3032 | GTTCAATCATATATCCATCCATC | 4296 | GATGGATGGATATATGATTGAAC |
| 3033 | AGTTCAATCATATATCCATCCAT | 4297 | ATGGATGGATATATGATTGAACT |
| 3034 | TAGTTCAATCATATATCCATCCA | 4298 | TGGATGGATATATGATTGAACTA |
| 3035 | ATAGTTCAATCATATATCCATCC | 4299 | GGATGGATATATGATTGAACTAT |
| 3036 | AATAGTTCAATCATATATCCATC | 4300 | GATGGATATATGATTGAACTATT |
| 3037 | CAATAGTTCAATCATATATCCAT | 4301 | ATGGATATATGATTGAACTATTG |
| 3038 | TCAATAGTTCAATCATATATCCA | 4302 | TGGATATATGATTGAACTATTGA |
| 3039 | TTCAATAGTTCAATCATATATCC | 4303 | GGATATATGATTGAACTATTGAA |
| 3040 | TTTCAATAGTTCAATCATATATC | 4304 | GATATATGATTGAACTATTGAAA |
| 3041 | CTTTCAATAGTTCAATCATATAT | 4305 | ATATATGATTGAACTATTGAAAG |
| 3042 | ACTTTCAATAGTTCAATCATATA | 4306 | TATATGATTGAACTATTGAAAGT |
| 3043 | TACTTTCAATAGTTCAATCATAT | 4307 | ATATGATTGAACTATTGAAAGTA |
| 3044 | ATACTTTCAATAGTTCAATCATA | 4308 | TATGATTGAACTATTGAAAGTAT |
| 3045 | TATACTTTCAATAGTTCAATCAT | 4309 | ATGATTGAACTATTGAAAGTATA |
| 3046 | CTATACTTTCAATAGTTCAATCA | 4310 | TGATTGAACTATTGAAAGTATAG |
| 3047 | TCTATACTTTCAATAGTTCAATC | 4311 | GATTGAACTATTGAAAGTATAGA |
| 3048 | ATCTATACTTTCAATAGTTCAAT | 4312 | ATTGAACTATTGAAAGTATAGAT |
| 3049 | CATCTATACTTTCAATAGTTCAA | 4313 | TTGAACTATTGAAAGTATAGATG |
| 3050 | ACATCTATACTTTCAATAGTTCA | 4314 | TGAACTATTGAAAGTATAGATGT |
| 3051 | TACATCTATACTTTCAATAGTTC | 4315 | GAACTATTGAAAGTATAGATGTA |
| 3052 | ATACATCTATACTTTCAATAGTT | 4316 | AACTATTGAAAGTATAGATGTAT |
| 3053 | CATACATCTATACTTTCAATAGT | 4317 | ACTATTGAAAGTATAGATGTATG |
| 3054 | CCATACATCTATACTTTCAATAG | 4318 | CTATTGAAAGTATAGATGTATGG |
| 3055 | TCCATACATCTATACTTTCAATA | 4319 | TATTGAAAGTATAGATGTATGGA |
| 3056 | ATCCATACATCTATACTTTCAAT | 4320 | ATTGAAAGTATAGATGTATGGAT |
| 3057 | CATCCATACATCTATACTTTCAA | 4321 | TTGAAAGTATAGATGTATGGATG |
| 3058 | CCATCCATACATCTATACTTTCA | 4322 | TGAAAGTATAGATGTATGGATGG |
| 3059 | CCCATCCATACATCTATACTTTC | 4323 | GAAAGTATAGATGTATGGATGGG |
| 3060 | ACCCATCCATACATCTATACTTT | 4324 | AAAGTATAGATGTATGGATGGGT |
| 3061 | CACCCATCCATACATCTATACTT | 4325 | AAGTATAGATGTATGGATGGGTG |
| 3062 | TCACCCATCCATACATCTATACT | 4326 | AGTATAGATGTATGGATGGGTGA |
| 3063 | TTCACCCATCCATACATCTATAC | 4327 | GTATAGATGTATGGATGGGTGAA |
| 3064 | ATTCACCCATCCATACATCTATA | 4328 | TATAGATGTATGGATGGGTGAAT |
| 3065 | AATTCACCCATCCATACATCTAT | 4329 | ATAGATGTATGGATGGGTGAATT |
| 3067 | CAAATTCACCCATCCATACATCT | 4331 | AGATGTATGGATGGGTGAATTTG |
| 3068 | CCAAATTCACCCATCCATACATC | 4332 | GATGTATGGATGGGTGAATTTGG |
| 3069 | CCCAAATTCACCCATCCATACAT | 4333 | ATGTATGGATGGGTGAATTTGGG |
| 3070 | CCCCAAATTCACCCATCCATACA | 4334 | TGTATGGATGGGTGAATTTGGGG |
| 3071 | CCCCCAAATTCACCCATCCATAC | 4335 | GTATGGATGGGTGAATTTGGGGG |
| 3072 | ACCCCCAAATTCACCCATCCATA | 4336 | TATGGATGGGTGAATTTGGGGGT |
| 3073 | TACCCCCAAATTCACCCATCCAT | 4337 | ATGGATGGGTGAATTTGGGGGTA |
| 3074 | TTACCCCCAAATTCACCCATCCA | 4338 | TGGATGGGTGAATTTGGGGGTAA |
| 3075 | ATTACCCCCAAATTCACCCATCC | 4339 | GGATGGGTGAATTTGGGGGTAAT |
| 3076 | AATTACCCCCAAATTCACCCATC | 4340 | GATGGGTGAATTTGGGGGTAATT |
| 3077 | CAATTACCCCCAAATTCACCCAT | 4341 | ATGGGTGAATTTGGGGGTAATTG |
| 3078 | ACAATTACCCCCAAATTCACCCA | 4342 | TGGGTGAATTTGGGGGTAATTGT |
| 3079 | AACAATTACCCCCAAATTCACCC | 4343 | GGGTGAATTTGGGGGTAATTGTT |
| 3080 | TAACAATTACCCCCAAATTCACC | 4344 | GGTGAATTTGGGGGTAATTGTTA |
| 3081 | CTAACAATTACCCCCAAATTCAC | 4345 | GTGAATTTGGGGGTAATTGTTAG |
| 3082 | TCTAACAATTACCCCCAAATTCA | 4346 | TGAATTTGGGGGTAATTGTTAGA |
| 3083 | ATCTAACAATTACCCCCAAATTC | 4347 | GAATTTGGGGGTAATTGTTAGAT |
| 3084 | CATCTAACAATTACCCCCAAATT | 4348 | AATTTGGGGGTAATTGTTAGATG |
| 3085 | TCATCTAACAATTACCCCCAAAT | 4349 | ATTTGGGGGTAATTGTTAGATGA |
| 3086 | ATCATCTAACAATTACCCCCAAA | 4350 | TTTGGGGGTAATTGTTAGATGAT |
| 3087 | CATCATCTAACAATTACCCCCAA | 4351 | TTGGGGGTAATTGTTAGATGATG |
| 3088 | CCATCATCTAACAATTACCCCCA | 4352 | TGGGGGTAATTGTTAGATGATGG |
| 3089 | TCCATCATCTAACAATTACCCCC | 4353 | GGGGGTAATTGTTAGATGATGGA |
| 3090 | ATCCATCATCTAACAATTACCCC | 4354 | GGGGTAATTGTTAGATGATGGAT |
| 3091 | CATCCATCATCTAACAATTACCC | 4355 | GGGTAATTGTTAGATGATGGATG |
| 3092 | TCATCCATCATCTAACAATTACC | 4356 | GGTAATTGTTAGATGATGGATGA |
| 3093 | CTCATCCATCATCTAACAATTAC | 4357 | GTAATTGTTAGATGATGGATGAG |
| 3094 | ACTCATCCATCATCTAACAATTA | 4358 | TAATTGTTAGATGATGGATGAGT |
| 3095 | TACTCATCCATCATCTAACAATT | 4359 | AATTGTTAGATGATGGATGAGTA |
| 3096 | ATACTCATCCATCATCTAACAAT | 4360 | ATTGTTAGATGATGGATGAGTAT |
| 3097 | TATACTCATCCATCATCTAACAA | 4361 | TTGTTAGATGATGGATGAGTATA |
| 3098 | CTATACTCATCCATCATCTAACA | 4362 | TGTTAGATGATGGATGAGTATAG |
| 3099 | TCTATACTCATCCATCATCTAAC | 4363 | GTTAGATGATGGATGAGTATAGA |
| 3100 | ATCTATACTCATCCATCATCTAA | 4364 | TTAGATGATGGATGAGTATAGAT |
| 3101 | CATCTATACTCATCCATCATCTA | 4365 | TAGATGATGGATGAGTATAGATG |
| 3102 | TCATCTATACTCATCCATCATCT | 4366 | AGATGATGGATGAGTATAGATGA |
| 3103 | TTCATCTATACTCATCCATCATC | 4367 | GATGATGGATGAGTATAGATGAA |
| 3104 | ATTCATCTATACTCATCCATCAT | 4368 | ATGATGGATGAGTATAGATGAAT |
| 3105 | CATTCATCTATACTCATCCATCA | 4369 | TGATGGATGAGTATAGATGAATG |
| 3106 | TCATTCATCTATACTCATCCATC | 4370 | GATGGATGAGTATAGATGAATGA |
| 3107 | ATCATTCATCTATACTCATCCAT | 4371 | ATGGATGAGTATAGATGAATGAT |
| 3108 | CATCATTCATCTATACTCATCCA | 4372 | TGGATGAGTATAGATGAATGATG |
| 3110 | TCCATCATTCATCTATACTCATC | 4374 | GATGAGTATAGATGAATGATGGA |
| 3111 | ATCCATCATTCATCTATACTCAT | 4375 | ATGAGTATAGATGAATGATGGAT |
| 3112 | CATCCATCATTCATCTATACTCA | 4376 | TGAGTATAGATGAATGATGGATG |
| 3113 | CCATCCATCATTCATCTATACTC | 4377 | GAGTATAGATGAATGATGGATGG |
| 3114 | TCCATCCATCATTCATCTATACT | 4378 | AGTATAGATGAATGATGGATGGA |
| 3115 | ATCCATCCATCATTCATCTATAC | 4379 | GTATAGATGAATGATGGATGGAT |
| 3116 | TATCCATCCATCATTCATCTATA | 4380 | TATAGATGAATGATGGATGGATA |
| 3117 | TTATCCATCCATCATTCATCTAT | 4381 | ATAGATGAATGATGGATGGATAA |
| 3118 | GTTATCCATCCATCATTCATCTA | 4382 | TAGATGAATGATGGATGGATAAC |
| 3119 | AGTTATCCATCCATCATTCATCT | 4383 | AGATGAATGATGGATGGATAACT |
| 3120 | AAGTTATCCATCCATCATTCATC | 4384 | GATGAATGATGGATGGATAACTT |
| 3121 | CAAGTTATCCATCCATCATTCAT | 4385 | ATGAATGATGGATGGATAACTTG |
| 3122 | TCAAGTTATCCATCCATCATTCA | 4386 | TGAATGATGGATGGATAACTTGA |
| 3123 | ATCAAGTTATCCATCCATCATTC | 4387 | GAATGATGGATGGATAACTTGAT |
| 3124 | CATCAAGTTATCCATCCATCATT | 4388 | AATGATGGATGGATAACTTGATG |
| 3125 | TCATCAAGTTATCCATCCATCAT | 4389 | ATGATGGATGGATAACTTGATGA |
| 3126 | CTCATCAAGTTATCCATCCATCA | 4390 | TGATGGATGGATAACTTGATGAG |
| 3127 | ACTCATCAAGTTATCCATCCATC | 4391 | GATGGATGGATAACTTGATGAGT |
| 3128 | CACTCATCAAGTTATCCATCCAT | 4392 | ATGGATGGATAACTTGATGAGTG |
| 3129 | CCACTCATCAAGTTATCCATCCA | 4393 | TGGATGGATAACTTGATGAGTGG |
| 3130 | TCCACTCATCAAGTTATCCATCC | 4394 | GGATGGATAACTTGATGAGTGGA |
| 3131 | ATCCACTCATCAAGTTATCCATC | 4395 | GATGGATAACTTGATGAGTGGAT |
| 3132 | TATCCACTCATCAAGTTATCCAT | 4396 | ATGGATAACTTGATGAGTGGATA |
| 3133 | CTATCCACTCATCAAGTTATCCA | 4397 | TGGATAACTTGATGAGTGGATAG |
| 3134 | TCTATCCACTCATCAAGTTATCC | 4398 | GGATAACTTGATGAGTGGATAGA |
| 3135 | ATCTATCCACTCATCAAGTTATC | 4399 | GATAACTTGATGAGTGGATAGAT |
| 3136 | TATCTATCCACTCATCAAGTTAT | 4400 | ATAACTTGATGAGTGGATAGATA |
| 3137 | CTATCTATCCACTCATCAAGTTA | 4401 | TAACTTGATGAGTGGATAGATAG |
| 3138 | TCTATCTATCCACTCATCAAGTT | 4402 | AACTTGATGAGTGGATAGATAGA |
| 3139 | ATCTATCTATCCACTCATCAAGT | 4403 | ACTTGATGAGTGGATAGATAGAT |
| 3140 | AATCTATCTATCCACTCATCAAG | 4404 | CTTGATGAGTGGATAGATAGATT |
| 3141 | CAATCTATCTATCCACTCATCAA | 4405 | TTGATGAGTGGATAGATAGATTG |
| 3142 | GCAATCTATCTATCCACTCATCA | 4406 | TGATGAGTGGATAGATAGATTGC |
| 3143 | AGCAATCTATCTATCCACTCATC | 4407 | GATGAGTGGATAGATAGATTGCT |
| 3144 | CAGCAATCTATCTATCCACTCAT | 4408 | ATGAGTGGATAGATAGATTGCTG |
| 3145 | CCAGCAATCTATCTATCCACTCA | 4409 | TGAGTGGATAGATAGATTGCTGG |
| 3146 | TCCAGCAATCTATCTATCCACTC | 4410 | GAGTGGATAGATAGATTGCTGGA |
| 3147 | ATCCAGCAATCTATCTATCCACT | 4411 | AGTGGATAGATAGATTGCTGGAT |
| 3148 | TATCCAGCAATCTATCTATCCAC | 4412 | GTGGATAGATAGATTGCTGGATA |
| 3149 | CTATCCAGCAATCTATCTATCCA | 4413 | TGGATAGATAGATTGCTGGATAG |
| 3150 | TCTATCCAGCAATCTATCTATCC | 4414 | GGATAGATAGATTGCTGGATAGA |
| 3151 | ATCTATCCAGCAATCTATCTATC | 4415 | GATAGATAGATTGCTGGATAGAT |
| 3153 | TCATCTATCCAGCAATCTATCTA | 4417 | TAGATAGATTGCTGGATAGATGA |
| 3154 | ATCATCTATCCAGCAATCTATCT | 4418 | AGATAGATTGCTGGATAGATGAT |
| 3155 | AATCATCTATCCAGCAATCTATC | 4419 | GATAGATTGCTGGATAGATGATT |
| 3156 | CAATCATCTATCCAGCAATCTAT | 4420 | ATAGATTGCTGGATAGATGATTG |
| 3157 | TCAATCATCTATCCAGCAATCTA | 4421 | TAGATTGCTGGATAGATGATTGA |
| 3158 | GTCAATCATCTATCCAGCAATCT | 4422 | AGATTGCTGGATAGATGATTGAC |
| 3159 | AGTCAATCATCTATCCAGCAATC | 4423 | GATTGCTGGATAGATGATTGACT |
| 3160 | CAGTCAATCATCTATCCAGCAAT | 4424 | ATTGCTGGATAGATGATTGACTG |
| 3161 | CCAGTCAATCATCTATCCAGCAA | 4425 | TTGCTGGATAGATGATTGACTGG |
| 3162 | CCCAGTCAATCATCTATCCAGCA | 4426 | TGCTGGATAGATGATTGACTGGG |
| 3163 | ACCCAGTCAATCATCTATCCAGC | 4427 | GCTGGATAGATGATTGACTGGGT |
| 3164 | CACCCAGTCAATCATCTATCCAG | 4428 | CTGGATAGATGATTGACTGGGTG |
| 3165 | CCACCCAGTCAATCATCTATCCA | 4429 | TGGATAGATGATTGACTGGGTGG |
| 3166 | TCCACCCAGTCAATCATCTATCC | 4430 | GGATAGATGATTGACTGGGTGGA |
| 3167 | ATCCACCCAGTCAATCATCTATC | 4431 | GATAGATGATTGACTGGGTGGAT |
| 3168 | TATCCACCCAGTCAATCATCTAT | 4432 | ATAGATGATTGACTGGGTGGATA |
| 3169 | CTATCCACCCAGTCAATCATCTA | 4433 | TAGATGATTGACTGGGTGGATAG |
| 3170 | TCTATCCACCCAGTCAATCATCT | 4434 | AGATGATTGACTGGGTGGATAGA |
| 3171 | ATCTATCCACCCAGTCAATCATC | 4435 | GATGATTGACTGGGTGGATAGAT |
| 3172 | CATCTATCCACCCAGTCAATCAT | 4436 | ATGATTGACTGGGTGGATAGATG |
| 3173 | TCATCTATCCACCCAGTCAATCA | 4437 | TGATTGACTGGGTGGATAGATGA |
| 3174 | TTCATCTATCCACCCAGTCAATC | 4438 | GATTGACTGGGTGGATAGATGAA |
| 3175 | TTTCATCTATCCACCCAGTCAAT | 4439 | ATTGACTGGGTGGATAGATGAAA |
| 3176 | ATTTCATCTATCCACCCAGTCAA | 4440 | TTGACTGGGTGGATAGATGAAAT |
| 3177 | CATTTCATCTATCCACCCAGTCA | 4441 | TGACTGGGTGGATAGATGAAATG |
| 3178 | ACATTTCATCTATCCACCCAGTC | 4442 | GACTGGGTGGATAGATGAAATGT |
| 3179 | AACATTTCATCTATCCACCCAGT | 4443 | ACTGGGTGGATAGATGAAATGTT |
| 3180 | CAACATTTCATCTATCCACCCAG | 4444 | CTGGGTGGATAGATGAAATGTTG |
| 3181 | CCAACATTTCATCTATCCACCCA | 4445 | TGGGTGGATAGATGAAATGTTGG |
| 3182 | TCCAACATTTCATCTATCCACCC | 4446 | GGGTGGATAGATGAAATGTTGGA |
| 3183 | ATCCAACATTTCATCTATCCACC | 4447 | GGTGGATAGATGAAATGTTGGAT |
| 3184 | CATCCAACATTTCATCTATCCAC | 4448 | GTGGATAGATGAAATGTTGGATG |
| 3185 | TCATCCAACATTTCATCTATCCA | 4449 | TGGATAGATGAAATGTTGGATGA |
| 3186 | CTCATCCAACATTTCATCTATCC | 4450 | GGATAGATGAAATGTTGGATGAG |
| 3187 | GCTCATCCAACATTTCATCTATC | 4451 | GATAGATGAAATGTTGGATGAGC |
| 3188 | TGCTCATCCAACATTTCATCTAT | 4452 | ATAGATGAAATGTTGGATGAGCA |
| 3189 | CTGCTCATCCAACATTTCATCTA | 4453 | TAGATGAAATGTTGGATGAGCAG |
| 3190 | TCTGCTCATCCAACATTTCATCT | 4454 | AGATGAAATGTTGGATGAGCAGA |
| 3191 | ATCTGCTCATCCAACATTTCATC | 4455 | GATGAAATGTTGGATGAGCAGAT |
| 3192 | AATCTGCTCATCCAACATTTCAT | 4456 | ATGAAATGTTGGATGAGCAGATT |
| 3193 | TAATCTGCTCATCCAACATTTCA | 4457 | TGAAATGTTGGATGAGCAGATTA |
| 3194 | TTAATCTGCTCATCCAACATTTC | 4458 | GAAATGTTGGATGAGCAGATTAA |
| 3196 | ACTTAATCTGCTCATCCAACATT | 4460 | AATGTTGGATGAGCAGATTAAGT |
| 3197 | AACTTAATCTGCTCATCCAACAT | 4461 | ATGTTGGATGAGCAGATTAAGTT |
| 3198 | CAACTTAATCTGCTCATCCAACA | 4462 | TGTTGGATGAGCAGATTAAGTTG |
| 3199 | ACAACTTAATCTGCTCATCCAAC | 4463 | GTTGGATGAGCAGATTAAGTTGT |
| 3200 | TACAACTTAATCTGCTCATCCAA | 4464 | TTGGATGAGCAGATTAAGTIGTA |
| 3201 | ATACAACTTAATCTGCTCATCCA | 4465 | TGGATGAGCAGATTAAGTIGTAT |
| 3202 | AATACAACTTAATCTGCTCATCC | 4466 | GGATGAGCAGATTAAGTTGTATT |
| 3203 | CAATACAACTTAATCTGCTCATC | 4467 | GATGAGCAGATTAAGTTGTATTG |
| 3204 | CCAATACAACTTAATCTGCTCAT | 4468 | ATGAGCAGATTAAGTTGTATTGG |
| 3205 | TCCAATACAACTTAATCTGCTCA | 4469 | TGAGCAGATTAAGTTGTATTGGA |
| 3206 | ATCCAATACAACTTAATCTGCTC | 4470 | GAGCAGATTAAGTTGTATTGGAT |
| 3207 | CATCCAATACAACTTAATCTGCT | 4471 | AGCAGATTAAGTIGTATTGGATG |
| 3208 | CCATCCAATACAACTTAATCTGC | 4472 | GCAGATTAAGTTGTATTGGATGG |
| 3209 | CCCATCCAATACAACTTAATCTG | 4473 | CAGATTAAGTTGTATTGGATGGG |
| 3210 | TCCCATCCAATACAACTTAATCT | 4474 | AGATTAAGTTGTATTGGATGGGA |
| 3211 | ATCCCATCCAATACAACTTAATC | 4475 | GATTAAGTTGTATTGGATGGGAT |
| 3212 | CATCCCATCCAATACAACTTAAT | 4476 | ATTAAGTTGTATTGGATGGGATG |
| 3213 | CCATCCCATCCAATACAACTTAA | 4477 | TTAAGTTGTATTGGATGGGATGG |
| 3214 | TCCATCCCATCCAATACAACTTA | 4478 | TAAGTTGTATTGGATGGGATGGA |
| 3215 | ATCCATCCCATCCAATACAACTT | 4479 | AAGTTGTATTGGATGGGATGGAT |
| 3216 | CATCCATCCCATCCAATACAACT | 4480 | AGTTGTATTGGATGGGATGGATG |
| 3217 | CCATCCATCCCATCCAATACAAC | 4481 | GTTGTATTGGATGGGATGGATGG |
| 3218 | TCCATCCATCCCATCCAATACAA | 4482 | TTGTATTGGATGGGATGGATGGA |
| 3219 | TTCCATCCATCCCATCCAATACA | 4483 | TGTATTGGATGGGATGGATGGAA |
| 3220 | CTTCCATCCATCCCATCCAATAC | 4484 | GTATTGGATGGGATGGATGGAAG |
| 3221 | ACTTCCATCCATCCCATCCAATA | 4485 | TATTGGATGGGATGGATGGAAGT |
| 3222 | CACTTCCATCCATCCCATCCAAT | 4486 | ATTGGATGGGATGGATGGAAGTG |
| 3223 | ACACTTCCATCCATCCCATCCAA | 4487 | TTGGATGGGATGGATGGAAGTGT |
| 3224 | CACACTTCCATCCATCCCATCCA | 4488 | TGGATGGGATGGATGGAAGTGTG |
| 3225 | CCACACTTCCATCCATCCCATCC | 4489 | GGATGGGATGGATGGAAGTGTGG |
| 3226 | ACCACACTTCCATCCATCCCATC | 4490 | GATGGGATGGATGGAAGTGTGGT |
| 3227 | AACCACACTTCCATCCATCCCAT | 4491 | ATGGGATGGATGGAAGTGTGGTT |
| 3228 | CAACCACACTTCCATCCATCCCA | 4492 | TGGGATGGATGGAAGTGTGGTTG |
| 3229 | TCAACCACACTTCCATCCATCCC | 4493 | GGGATGGATGGAAGTGTGGTTGA |
| 3230 | CTCAACCACACTTCCATCCATCC | 4494 | GGATGGATGGAAGTGTGGTTGAG |
| 3231 | ACTCAACCACACTTCCATCCATC | 4495 | GATGGATGGAAGTGTGGTTGAGT |
| 3232 | AACTCAACCACACTTCCATCCAT | 4496 | ATGGATGGAAGTGTGGTTGAGTT |
| 3233 | TAACTCAACCACACTTCCATCCA | 4497 | TGGATGGAAGTGTGGTTGAGTTA |
| 3234 | ATAACTCAACCACACTTCCATCC | 4498 | GGATGGAAGTGTGGTTGAGTTAT |
| 3235 | AATAACTCAACCACACTTCCATC | 4499 | GATGGAAGTGTGGTTGAGTTATT |
| 3236 | TAATAACTCAACCACACTTCCAT | 4500 | ATGGAAGTGTGGTTGAGTTATTA |
| 3237 | CTAATAACTCAACCACACTTCCA | 4501 | TGGAAGTGTGGTTGAGTTATTAG |
| 3239 | TTCTAATAACTCAACCACACTTC | 4503 | GAAGTGTGGTTGAGTTATTAGAA |
| 3240 | CTTCTAATAACTCAACCACACTT | 4504 | AAGTGTGGTTGAGTTATTAGAAG |
| 3241 | CCTTCTAATAACTCAACCACACT | 4505 | AGTGTGGTTGAGTTATTAGAAGG |
| 3242 | TCCTTCTAATAACTCAACCACAC | 4506 | GTGTGGTTGAGTTATTAGAAGGA |
| 3243 | TTCCTTCTAATAACTCAACCACA | 4507 | TGTGGTTGAGTTATTAGAAGGAA |
| 3244 | CTTCCTTCTAATAACTCAACCAC | 4508 | GTGGTTGAGTTATTAGAAGGAAG |
| 3245 | TCTTCCTTCTAATAACTCAACCA | 4509 | TGGTTGAGTTATTAGAAGGAAGA |
| 3246 | ATCTTCCTTCTAATAACTCAACC | 4510 | GGTTGAGTTATTAGAAGGAAGAT |
| 3247 | AATCTTCCTTCTAATAACTCAAC | 4511 | GTTGAGTTATTAGAAGGAAGATT |
| 3248 | CAATCTTCCTTCTAATAACTCAA | 4512 | TTGAGTTATTAGAAGGAAGATTG |
| 3249 | TCAATCTTCCTTCTAATAACTCA | 4513 | TGAGTTATTAGAAGGAAGATTGA |
| 3250 | CTCAATCTTCCTTCTAATAACTC | 4514 | GAGTTATTAGAAGGAAGATTGAG |
| 3251 | ACTCAATCTTCCTTCTAATAACT | 4515 | AGTTATTAGAAGGAAGATTGAGT |
| 3252 | TACTCAATCTTCCTTCTAATAAC | 4516 | GTTATTAGAAGGAAGATTGAGTA |
| 3253 | CTACTCAATCTTCCTTCTAATAA | 4517 | TTATTAGAAGGAAGATTGAGTAG |
| 3254 | TCTACTCAATCTTCCTTCTAATA | 4518 | TATTAGAAGGAAGATTGAGTAGA |
| 3255 | ATCTACTCAATCTTCCTTCTAAT | 4519 | ATTAGAAGGAAGATTGAGTAGAT |
| 3256 | TATCTACTCAATCTTCCTTCTAA | 4520 | TTAGAAGGAAGATTGAGTAGATA |
| 3257 | CTATCTACTCAATCTTCCTTCTA | 4521 | TAGAAGGAAGATTGAGTAGATAG |
| 3258 | CCTATCTACTCAATCTTCCTTCT | 4522 | AGAAGGAAGATTGAGTAGATAGG |
| 3259 | ACCTATCTACTCAATCTTCCTTC | 4523 | GAAGGAAGATTGAGTAGATAGGT |
| 3260 | CACCTATCTACTCAATCTTCCTT | 4524 | AAGGAAGATTGAGTAGATAGGTG |
| 3261 | TCACCTATCTACTCAATCTTCCT | 4525 | AGGAAGATTGAGTAGATAGGTGA |
| 3262 | TTCACCTATCTACTCAATCTTCC | 4526 | GGAAGATTGAGTAGATAGGTGAA |
| 3263 | ATTCACCTATCTACTCAATCTTC | 4527 | GAAGATTGAGTAGATAGGTGAAT |
| 3264 | AATTCACCTATCTACTCAATCTT | 4528 | AAGATTGAGTAGATAGGTGAATT |
| 3265 | AAATTCACCTATCTACTCAATCT | 4529 | AGATTGAGTAGATAGGTGAATTT |
| 3266 | CAAATTCACCTATCTACTCAATC | 4530 | GATTGAGTAGATAGGTGAATTTG |
| 3267 | ACAAATTCACCTATCTACTCAAT | 4531 | ATTGAGTAGATAGGTGAATTTGT |
| 3268 | AACAAATTCACCTATCTACTCAA | 4532 | TTGAGTAGATAGGTGAATTTGTT |
| 3269 | CAACAAATTCACCTATCTACTCA | 4533 | TGAGTAGATAGGTGAATTTGTTG |
| 3270 | TCAACAAATTCACCTATCTACTC | 4534 | GAGTAGATAGGTGAATTTGTTGA |
| 3271 | ATCAACAAATTCACCTATCTACT | 4535 | AGTAGATAGGTGAATTTGTTGAT |
| 3272 | TATCAACAAATTCACCTATCTAC | 4536 | GTAGATAGGTGAATTTGTTGATA |
| 3273 | CTATCAACAAATTCACCTATCTA | 4537 | TAGATAGGTGAATTTGTTGATAG |
| 3274 | ACTATCAACAAATTCACCTATCT | 4538 | AGATAGGTGAATTTGTTGATAGT |
| 3275 | GACTATCAACAAATTCACCTATC | 4539 | GATAGGTGAATTTGTTGATAGTC |
| 3276 | TGACTATCAACAAATTCACCTAT | 4540 | ATAGGTGAATTTGTTGATAGTCA |
| 3277 | CTGACTATCAACAAATTCACCTA | 4541 | TAGGTGAATTTGTTGATAGTCAG |
| 3278 | TCTGACTATCAACAAATTCACCT | 4542 | AGGTGAATTTGTTGATAGTCAGA |
| 3279 | ATCTGACTATCAACAAATTCACC | 4543 | GGTGAATTTGTTGATAGTCAGAT |
| 3280 | CATCTGACTATCAACAAATTCAC | 4544 | GTGAATTTGTTGATAGTCAGATG |
| 3282 | CCCATCTGACTATCAACAAATTC | 4546 | GAATTTGTTGATAGTCAGATGGG |
| 3283 | ACCCATCTGACTATCAACAAATT | 4547 | AATTTGTTGATAGTCAGATGGGT |
| 3284 | TACCCATCTGACTATCAACAAAT | 4548 | ATTTGTTGATAGTCAGATGGGTA |
| 3285 | CTACCCATCTGACTATCAACAAA | 4549 | TTTGTTGATAGTCAGATGGGTAG |
| 3286 | TCTACCCATCTGACTATCAACAA | 4550 | TTGTTGATAGTCAGATGGGTAGA |
| 3287 | ATCTACCCATCTGACTATCAACA | 4551 | TGTTGATAGTCAGATGGGTAGAT |
| 3288 | TATCTACCCATCTGACTATCAAC | 4552 | GTTGATAGTCAGATGGGTAGATA |
| 3289 | CTATCTACCCATCTGACTATCAA | 4553 | TTGATAGTCAGATGGGTAGATAG |
| 3290 | CCTATCTACCCATCTGACTATCA | 4554 | TGATAGTCAGATGGGTAGATAGG |
| 3291 | ACCTATCTACCCATCTGACTATC | 4555 | GATAGTCAGATGGGTAGATAGGT |
| 3292 | TACCTATCTACCCATCTGACTAT | 4556 | ATAGTCAGATGGGTAGATAGGTA |
| 3293 | CTACCTATCTACCCATCTGACTA | 4557 | TAGTCAGATGGGTAGATAGGTAG |
| 3294 | TCTACCTATCTACCCATCTGACT | 4558 | AGTCAGATGGGTAGATAGGTAGA |
| 3295 | ATCTACCTATCTACCCATCTGAC | 4559 | GTCAGATGGGTAGATAGGTAGAT |
| 3296 | CATCTACCTATCTACCCATCTGA | 4560 | TCAGATGGGTAGATAGGTAGATG |
| 3297 | CCATCTACCTATCTACCCATCTG | 4561 | CAGATGGGTAGATAGGTAGATGG |
| 3298 | TCCATCTACCTATCTACCCATCT | 4562 | AGATGGGTAGATAGGTAGATGGA |
| 3299 | ATCCATCTACCTATCTACCCATC | 4563 | GATGGGTAGATAGGTAGATGGAT |
| 3300 | CATCCATCTACCTATCTACCCAT | 4564 | ATGGGTAGATAGGTAGATGGATG |
| 3301 | CCATCCATCTACCTATCTACCCA | 4565 | TGGGTAGATAGGTAGATGGATGG |
| 3302 | TCCATCCATCTACCTATCTACCC | 4566 | GGGTAGATAGGTAGATGGATGGA |
| 3303 | ATCCATCCATCTACCTATCTACC | 4567 | GGTAGATAGGTAGATGGATGGAT |
| 3304 | CATCCATCCATCTACCTATCTAC | 4568 | GTAGATAGGTAGATGGATGGATG |
| 3305 | CCATCCATCCATCTACCTATCTA | 4569 | TAGATAGGTAGATGGATGGATGG |
| 3306 | TCCATCCATCCATCTACCTATCT | 4570 | AGATAGGTAGATGGATGGATGGA |
| 3307 | ATCCATCCATCCATCTACCTATC | 4571 | GATAGGTAGATGGATGGATGGAT |
| 3308 | CATCCATCCATCCATCTACCTAT | 4572 | ATAGGTAGATGGATGGATGGATG |
| 3309 | CCATCCATCCATCCATCTACCTA | 4573 | TAGGTAGATGGATGGATGGATGG |
| 3310 | TCCATCCATCCATCCATCTACCT | 4574 | AGGTAGATGGATGGATGGATGGA |
| 3311 | ATCCATCCATCCATCCATCTACC | 4575 | GGTAGATGGATGGATGGATGGAT |
| 3312 | CATCCATCCATCCATCCATCTAC | 4576 | GTAGATGGATGGATGGATGGATG |
| 3313 | CCATCCATCCATCCATCCATCTA | 4577 | TAGATGGATGGATGGATGGATGG |
| 3314 | TCCATCCATCCATCCATCCATCT | 4578 | AGATGGATGGATGGATGGATGGA |
| 3315 | ATCCATCCATCCATCCATCCATC | 4579 | GATGGATGGATGGATGGATGGAT |
| 3316 | CATCCATCCATCCATCCATCCAT | 4580 | ATGGATGGATGGATGGATGGATG |
| 3317 | ACATCCATCCATCCATCCATCCA | 4581 | TGGATGGATGGATGGATGGATGT |
| 3318 | TACATCCATCCATCCATCCATCC | 4582 | GGATGGATGGATGGATGGATGTA |
| 3319 | ATACATCCATCCATCCATCCATC | 4583 | GATGGATGGATGGATGGATGTAT |
| 3320 | TATACATCCATCCATCCATCCAT | 4584 | ATGGATGGATGGATGGATGTATA |
| 3321 | CTATACATCCATCCATCCATCCA | 4585 | TGGATGGATGGATGGATGTATAG |
| 3322 | CCTATACATCCATCCATCCATCC | 4586 | GGATGGATGGATGGATGTATAGG |
| 3323 | GCCTATACATCCATCCATCCATC | 4587 | GATGGATGGATGGATGTATAGGC |
| 3325 | CTGCCTATACATCCATCCATCCA | 4589 | TGGATGGATGGATGTATAGGCAG |
| 3326 | TCTGCCTATACATCCATCCATCC | 4590 | GGATGGATGGATGTATAGGCAGA |
| 3327 | ATCTGCCTATACATCCATCCATC | 4591 | GATGGATGGATGTATAGGCAGAT |
| 3328 | CATCTGCCTATACATCCATCCAT | 4592 | ATGGATGGATGTATAGGCAGATG |
| 3329 | CCATCTGCCTATACATCCATCCA | 4593 | TGGATGGATGTATAGGCAGATGG |
| 3330 | TCCATCTGCCTATACATCCATCC | 4594 | GGATGGATGTATAGGCAGATGGA |
| 3331 | GTCCATCTGCCTATACATCCATC | 4595 | GATGGATGTATAGGCAGATGGAC |
| 3332 | TGTCCATCTGCCTATACATCCAT | 4596 | ATGGATGTATAGGCAGATGGACA |
| 3333 | TTGTCCATCTGCCTATACATCCA | 4597 | TGGATGTATAGGCAGATGGACAA |
| 3334 | TTTGTCCATCTGCCTATACATCC | 4598 | GGATGTATAGGCAGATGGACAAA |
| 3335 | ATTTGTCCATCTGCCTATACATC | 4599 | GATGTATAGGCAGATGGACAAAT |
| 3336 | CATTTGTCCATCTGCCTATACAT | 4600 | ATGTATAGGCAGATGGACAAATG |
| 3337 | CCATTTGTCCATCTGCCTATACA | 4601 | TGTATAGGCAGATGGACAAATGG |
| 3338 | TCCATTTGTCCATCTGCCTATAC | 4602 | GTATAGGCAGATGGACAAATGGA |
| 3339 | ATCCATTTGTCCATCTGCCTATA | 4603 | TATAGGCAGATGGACAAATGGAT |
| 3340 | CATCCATTTGTCCATCTGCCTAT | 4604 | ATAGGCAGATGGACAAATGGATG |
| 3341 | TCATCCATTTGTCCATCTGCCTA | 4605 | TAGGCAGATGGACAAATGGATGA |
| 3342 | TTCATCCATTTGTCCATCTGCCT | 4606 | AGGCAGATGGACAAATGGATGAA |
| 3343 | ATTCATCCATTTGTCCATCTGCC | 4607 | GGCAGATGGACAAATGGATGAAT |
| 3344 | CATTCATCCATTTGTCCATCTGC | 4608 | GCAGATGGACAAATGGATGAATG |
| 3345 | CCATTCATCCATTTGTCCATCTG | 4609 | CAGATGGACAAATGGATGAATGG |
| 3346 | CCCATTCATCCATTTGTCCATCT | 4610 | AGATGGACAAATGGATGAATGGG |
| 3347 | ACCCATTCATCCATTTGTCCATC | 4611 | GATGGACAAATGGATGAATGGGT |
| 3348 | CACCCATTCATCCATTTGTCCAT | 4612 | ATGGACAAATGGATGAATGGGTG |
| 3349 | CCACCCATTCATCCATTTGTCCA | 4613 | TGGACAAATGGATGAATGGGTGG |
| 3350 | CCCACCCATTCATCCATTTGTCC | 4614 | GGACAAATGGATGAATGGGTGGG |
| 3351 | ACCCACCCATTCATCCATTTGTC | 4615 | GACAAATGGATGAATGGGTGGGT |
| 3352 | CACCCACCCATTCATCCATTTGT | 4616 | ACAAATGGATGAATGGGTGGGTG |
| 3353 | CCACCCACCCATTCATCCATTTG | 4617 | CAAATGGATGAATGGGTGGGTGG |
| 3354 | TCCACCCACCCATTCATCCATTT | 4618 | AAATGGATGAATGGGTGGGTGGA |
| 3355 | ATCCACCCACCCATTCATCCATT | 4619 | AATGGATGAATGGGTGGGTGGAT |
| 3356 | CATCCACCCACCCATTCATCCAT | 4620 | ATGGATGAATGGGTGGGTGGATG |
| 335″ | TCATCCACCCACCCATTCATCCA | 4621 | TGGATGAATGGGTGGGTGGATGA |
| 3358 | TTCATCCACCCACCCATTCATCC | 4622 | GGATGAATGGGTGGGTGGATGAA |
| 3359 | ATTCATCCACCCACCCATTCATC | 4623 | GATGAATGGGTGGGTGGATGAAT |
| 3360 | CATTCATCCACCCACCCATTCAT | 4624 | ATGAATGGGTGGGTGGATGAATG |
| 3361 | CCATTCATCCACCCACCCATTCA | 4625 | TGAATGGGTGGGTGGATGAATGG |
| 3362 | TCCATTCATCCACCCACCCATTC | 4626 | GAATGGGTGGGTGGATGAATGGA |
| 3363 | TTCCATTCATCCACCCACCCATT | 4627 | AATGGGTGGGTGGATGAATGGAA |
| 3364 | CTTCCATTCATCCACCCACCCAT | 4628 | ATGGGTGGGTGGATGAATGGAAG |
| 3365 | CCTTCCATTCATCCACCCACCCA | 4629 | TGGGTGGGTGGATGAATGGAAGG |
| 3366 | TCCTTCCATTCATCCACCCACCC | 4630 | GGGTGGGTGGATGAATGGAAGGA |
| 3368 | CATCCTTCCATTCATCCACCCAC | 4632 | GTGGGTGGATGAATGGAAGGATG |
| 3369 | ACATCCTTCCATTCATCCACCCA | 4633 | TGGGTGGATGAATGGAAGGATGT |
| 3370 | CACATCCTTCCATTCATCCACCC | 4634 | GGGTGGATGAATGGAAGGATGTG |
| 3371 | ACACATCCTTCCATTCATCCACC | 4635 | GGTGGATGAATGGAAGGATGTGT |
| 3372 | CACACATCCTTCCATTCATCCAC | 4636 | GTGGATGAATGGAAGGATGTGTG |
| 3373 | CCACACATCCTTCCATTCATCCA | 4637 | TGGATGAATGGAAGGATGTGTGG |
| 3374 | ACCACACATCCTTCCATTCATCC | 4638 | GGATGAATGGAAGGATGTGTGGT |
| 3375 | AACCACACATCCTTCCATTCATC | 4639 | GATGAATGGAAGGATGTGTGGTT |
| 3376 | CAACCACACATCCTTCCATTCAT | 4640 | ATGAATGGAAGGATGTGTGGTTG |
| 3377 | TCAACCACACATCCTTCCATTCA | 4641 | TGAATGGAAGGATGTGTGGTTGA |
| 3378 | TTCAACCACACATCCTTCCATTC | 4642 | GAATGGAAGGATGTGTGGTTGAA |
| 3379 | GTTCAACCACACATCCTTCCATT | 4643 | AATGGAAGGATGTGTGGTTGAAC |
| 3380 | AGTTCAACCACACATCCTTCCAT | 4644 | ATGGAAGGATGTGTGGTTGAACT |
| 3381 | TAGTTCAACCACACATCCTTCCA | 4645 | TGGAAGGATGTGTGGTTGAACTA |
| 3382 | ATAGTTCAACCACACATCCTTCC | 4646 | GGAAGGATGTGTGGTTGAACTAT |
| 3383 | AATAGTTCAACCACACATCCTTC | 4647 | GAAGGATGTGTGGTTGAACTATT |
| 3384 | CAATAGTTCAACCACACATCCTT | 4648 | AAGGATGTGTGGTTGAACTATTG |
| 3385 | GCAATAGTTCAACCACACATCCT | 4649 | AGGATGTGTGGTTGAACTATTGC |
| 3386 | TGCAATAGTTCAACCACACATCC | 4650 | GGATGTGTGGTTGAACTATTGCA |
| 3387 | TTGCAATAGTTCAACCACACATC | 4651 | GATGTGTGGTTGAACTATTGCAA |
| 3388 | CTTGCAATAGTTCAACCACACAT | 4652 | ATGTGTGGTTGAACTATTGCAAG |
| 3389 | ACTTGCAATAGTTCAACCACACA | 4653 | TGTGTGGTTGAACTATTGCAAGT |
| 3390 | TACTTGCAATAGTTCAACCACAC | 4654 | GTGTGGTTGAACTATTGCAAGTA |
| 3391 | ATACTTGCAATAGTTCAACCACA | 4655 | TGTGGTTGAACTATTGCAAGTAT |
| 3392 | AATACTTGCAATAGTTCAACCAC | 4656 | GTGGTTGAACTATTGCAAGTATT |
| 3393 | CAATACTTGCAATAGTTCAACCA | 4657 | TGGTTGAACTATTGCAAGTATTG |
| 3394 | TCAATACTTGCAATAGTTCAACC | 4658 | GGTTGAACTATTGCAAGTATTGA |
| 3395 | ATCAATACTTGCAATAGTTCAAC | 4659 | GTTGAACTATTGCAAGTATTGAT |
| 3396 | TATCAATACTTGCAATAGTTCAA | 4660 | TTGAACTATTGCAAGTATTGATA |
| 3397 | TTATCAATACTTGCAATAGTTCA | 4661 | TGAACTATTGCAAGTATTGATAA |
| 3398 | ATTATCAATACTTGCAATAGTTC | 4662 | GAACTATTGCAAGTATTGATAAT |
| 3399 | AATTATCAATACTTGCAATAGTT | 4663 | AACTATTGCAAGTATTGATAATT |
| 3400 | CAATTATCAATACTTGCAATAGT | 4664 | ACTATTGCAAGTATTGATAATTG |
| 3401 | CCAATTATCAATACTTGCAATAG | 4665 | CTATTGCAAGTATTGATAATTGG |
| 3402 | CCCAATTATCAATACTTGCAATA | 4666 | TATTGCAAGTATTGATAATTGGG |
| 3403 | ACCCAATTATCAATACTTGCAAT | 4667 | ATTGCAAGTATTGATAATTGGGT |
| 3404 | AACCCAATTATCAATACTTGCAA | 4668 | TTGCAAGTATTGATAATTGGGTT |
| 3405 | GAACCCAATTATCAATACTTGCA | 4669 | TGCAAGTATTGATAATTGGGTTC |
| 3406 | TGAACCCAATTATCAATACTTGC | 4670 | GCAAGTATTGATAATTGGGTTCA |
| 3407 | ATGAACCCAATTATCAATACTTG | 4671 | CAAGTATTGATAATTGGGTTCAT |
| 3408 | TATGAACCCAATTATCAATACTT | 4672 | AAGTATTGATAATTGGGTTCATA |
| 3409 | TTATGAACCCAATTATCAATACT | 4673 | AGTATTGATAATTGGGTTCATAA |
| 3410 | ATTATGAACCCAATTATCAATAC | 4674 | GTATTGATAATTGGGTTCATAAT |
| 3411 | AATTATGAACCCAATTATCAATA | 4675 | TATTGATAATTGGGTTCATAATT |
| 3412 | AAATTATGAACCCAATTATCAAT | 4676 | ATTGATAATTGGGTTCATAATTT |
| 3413 | GAAATTATGAACCCAATTATCAA | 4677 | TTGATAATTGGGTTCATAATTTC |
| 3414 | AGAAATTATGAACCCAATTATCA | 4678 | TGATAATTGGGTTCATAATTTCT |
| 3415 | CAGAAATTATGAACCCAATTATC | 4679 | GATAATTGGGTTCATAATTTCTG |
| 3416 | TCAGAAATTATGAACCCAATTAT | 4680 | ATAATTGGGTTCATAATTTCTGA |
| 3417 | TTCAGAAATTATGAACCCAATTA | 4681 | TAATTGGGTTCATAATTTCTGAA |
| 3418 | ATTCAGAAATTATGAACCCAATT | 4682 | AATTGGGTTCATAATTTCTGAAT |
| 3419 | TATTCAGAAATTATGAACCCAAT | 4683 | ATTGGGTTCATAATTTCTGAATA |
| 3420 | ATATTCAGAAATTATGAACCCAA | 4684 | TTGGGTTCATAATTTCTGAATAT |
| 3421 | AATATTCAGAAATTATGAACCCA | 4685 | TGGGTTCATAATTTCTGAATATT |
| 3422 | AAATATTCAGAAATTATGAACCC | 4686 | GGGTTCATAATTTCTGAATATTT |
| 3423 | TAAATATTCAGAAATTATGAACC | 4687 | GGTTCATAATTTCTGAATATTTA |
| 3424 | CTAAATATTCAGAAATTATGAAC | 4688 | GTTCATAATTTCTGAATATTTAG |
| 3425 | TCTAAATATTCAGAAATTATGAA | 4689 | TTCATAATTTCTGAATATTTAGA |
| 3426 | ATCTAAATATTCAGAAATTATGA | 4690 | TCATAATTTCTGAATATTTAGAT |
| 3427 | CATCTAAATATTCAGAAATTATG | 4691 | CATAATTTCTGAATATTTAGATG |
| 3428 | CCATCTAAATATTCAGAAATTAT | 4692 | ATAATTTCTGAATATTTAGATGG |
| 3429 | TCCATCTAAATATTCAGAAATTA | 4693 | TAATTTCTGAATATTTAGATGGA |
| 3430 | ATCCATCTAAATATTCAGAAATT | 4694 | AATTTCTGAATATTTAGATGGAT |
| 3431 | CATCCATCTAAATATTCAGAAAT | 4695 | ATTTCTGAATATTTAGATGGATG |
| 3432 | CCATCCATCTAAATATTCAGAAA | 4696 | TTTCTGAATATTTAGATGGATGG |
| 3433 | ACCATCCATCTAAATATTCAGAA | 4697 | TTCTGAATATTTAGATGGATGGT |
| 3434 | AACCATCCATCTAAATATTCAGA | 4698 | TCTGAATATTTAGATGGATGGTT |
| 3435 | CAACCATCCATCTAAATATTCAG | 4699 | CTGAATATTTAGATGGATGGTTG |
| 3436 | ACAACCATCCATCTAAATATTCA | 4700 | TGAATATTTAGATGGATGGTTGT |
| 3437 | CACAACCATCCATCTAAATATTC | 4701 | GAATATTTAGATGGATGGTTGTG |
| 3438 | TCACAACCATCCATCTAAATATT | 4702 | AATATTTAGATGGATGGTTGTGA |
| 3439 | CTCACAACCATCCATCTAAATAT | 4703 | ATATTTAGATGGATGGTTGTGAG |
| 3440 | ACTCACAACCATCCATCTAAATA | 4704 | TATTTAGATGGATGGTTGTGAGT |
| 3441 | CACTCACAACCATCCATCTAAAT | 4705 | ATTTAGATGGATGGTTGTGAGTG |
| 3442 | CCACTCACAACCATCCATCTAAA | 4706 | TTTAGATGGATGGTTGTGAGTGG |
| 3443 | GCCACTCACAACCATCCATCTAA | 4707 | TTAGATGGATGGTTGTGAGTGGC |
| 3444 | AGCCACTCACAACCATCCATCTA | 4708 | TAGATGGATGGTTGTGAGTGGCT |
| 3445 | CAGCCACTCACAACCATCCATCT | 4709 | AGATGGATGGTTGTGAGTGGCTG |
| 3446 | CCAGCCACTCACAACCATCCATC | 4710 | GATGGATGGTTGTGAGTGGCTGG |
| 3447 | ACCAGCCACTCACAACCATCCAT | 4711 | ATGGATGGTTGTGAGTGGCTGGT |
| 3448 | CACCAGCCACTCACAACCATCCA | 4712 | TGGATGGTTGTGAGTGGCTGGTG |
| 3449 | CCACCAGCCACTCACAACCATCC | 4713 | GGATGGTTGTGAGTGGCTGGTGG |
| 3450 | TCCACCAGCCACTCACAACCATC | 4714 | GATGGTTGTGAGTGGCTGGTGGA |
| 3451 | GTCCACCAGCCACTCACAACCAT | 4715 | ATGGTTGTGAGTGGCTGGTGGAC |
| 3452 | TGTCCACCAGCCACTCACAACCA | 4716 | TGGTTGTGAGTGGCTGGTGGACA |
| 3454 | TCTGTCCACCAGCCACTCACAAC | 4718 | GTTGTGAGTGGCTGGTGGACAGA |
| 3455 | GTCTGTCCACCAGCCACTCACAA | 4719 | TTGTGAGTGGCTGGTGGACAGAC |
| 3456 | CGTCTGTCCACCAGCCACTCACA | 4720 | TGTGAGTGGCTGGTGGACAGACG |
| 3457 | TCGTCTGTCCACCAGCCACTCAC | 4721 | GTGAGTGGCTGGTGGACAGACGA |
| 3458 | TTCGTCTGTCCACCAGCCACTCA | 4722 | TGAGTGGCTGGTGGACAGACGAA |
| 3459 | TTTCGTCTGTCCACCAGCCACTC | 4723 | GAGTGGCTGGTGGACAGACGAAA |
| 3460 | TTTTCGTCTGTCCACCAGCCACT | 4724 | AGTGGCTGGTGGACAGACGAAAA |
| 3461 | TTTTTCGTCTGTCCACCAGCCAC | 4725 | GTGGCTGGTGGACAGACGAAAAA |
| 3462 | ATTTTTCGTCTGTCCACCAGCCA | 4726 | TGGCTGGTGGACAGACGAAAAAT |
| 3463 | CATTTTTCGTCTGTCCACCAGCC | 4727 | GGCTGGTGGACAGACGAAAAATG |
| 3464 | CCATTTTTCGTCTGTCCACCAGC | 4728 | GCTGGTGGACAGACGAAAAATGG |
| 3465 | TCCATTTTTCGTCTGTCCACCAG | 4729 | CTGGTGGACAGACGAAAAATGGA |
| 3466 | ATCCATTTTTCGTCTGTCCACCA | 4730 | TGGTGGACAGACGAAAAATGGAT |
| 3467 | CATCCATTTTTCGTCTGTCCACC | 4731 | GGTGGACAGACGAAAAATGGATG |
| 3468 | CCATCCATTTTTCGTCTGTCCAC | 4732 | GTGGACAGACGAAAAATGGATGG |
| 3469 | ACCATCCATTTTTCGTCTGTCCA | 4733 | TGGACAGACGAAAAATGGATGGT |
| 3470 | AACCATCCATTTTTCGTCTGTCC | 4734 | GGACAGACGAAAAATGGATGGTT |
| 3471 | CAACCATCCATTTTTCGTCTGTC | 4735 | GACAGACGAAAAATGGATGGTTG |
| 3472 | CCAACCATCCATTTTTCGTCTGT | 4736 | ACAGACGAAAAATGGATGGTTGG |
| 3473 | TCCAACCATCCATTTTTCGTCTG | 4737 | CAGACGAAAAATGGATGGTTGGA |
| 3474 | ATCCAACCATCCATTTTTCGTCT | 4738 | AGACGAAAAATGGATGGTTGGAT |
| 3475 | TATCCAACCATCCATTTTTCGTC | 4739 | GACGAAAAATGGATGGTTGGATA |
| 3476 | TTATCCAACCATCCATTTTTCGT | 4740 | ACGAAAAATGGATGGTTGGATAA |
| 3477 | TTTATCCAACCATCCATTTTTCG | 4741 | CGAAAAATGGATGGTTGGATAAA |
| 3478 | ATTTATCCAACCATCCATTTTTC | 4742 | GAAAAATGGATGGTTGGATAAAT |
| 3479 | AATTTATCCAACCATCCATTTTT | 4743 | AAAAATGGATGGTTGGATAAATT |
| 3480 | CAATTTATCCAACCATCCATTTT | 4744 | AAAATGGATGGTTGGATAAATTG |
| 3481 | TCAATTTATCCAACCATCCATTT | 4745 | AAATGGATGGTTGGATAAATTGA |
| 3482 | ATCAATTTATCCAACCATCCATT | 4746 | AATGGATGGTTGGATAAATTGAT |
| 3483 | CATCAATTTATCCAACCATCCAT | 4747 | ATGGATGGTTGGATAAATTGATG |
| 3484 | CCATCAATTTATCCAACCATCCA | 4748 | TGGATGGTTGGATAAATTGATGG |
| 3485 | CCCATCAATTTATCCAACCATCC | 4749 | GGATGGTTGGATAAATTGATGGG |
| 3486 | ACCCATCAATTTATCCAACCATC | 4750 | GATGGTTGGATAAATTGATGGGT |
| 3487 | CACCCATCAATTTATCCAACCAT | 4751 | ATGGTTGGATAAATTGATGGGTG |
| 3488 | CCACCCATCAATTTATCCAACCA | 4752 | TGGTTGGATAAATTGATGGGTGG |
| 3489 | TCCACCCATCAATTTATCCAACC | 4753 | GGTTGGATAAATTGATGGGTGGA |
| 3490 | ATCCACCCATCAATTTATCCAAC | 4754 | GTTGGATAAATTGATGGGTGGAT |
| 3491 | CATCCACCCATCAATTTATCCAA | 4755 | TTGGATAAATTGATGGGTGGATG |
| 3492 | CCATCCACCCATCAATTTATCCA | 4756 | TGGATAAATTGATGGGTGGATGG |
| 3493 | TCCATCCACCCATCAATTTATCC | 4757 | GGATAAATTGATGGGTGGATGGA |
| 3494 | ATCCATCCACCCATCAATTTATC | 4758 | GATAAATTGATGGGTGGATGGAT |
| 3495 | CATCCATCCACCCATCAATTTAT | 4759 | ATAAATTGATGGGTGGATGGATG |
| 3497 | ACCATCCATCCACCCATCAATTT | 4761 | AAATTGATGGGTGGATGGATGGT |
| 3498 | AACCATCCATCCACCCATCAATT | 4762 | AATTGATGGGTGGATGGATGGTT |
| 3499 | CAACCATCCATCCACCCATCAAT | 4763 | ATTGATGGGTGGATGGATGGTTG |
| 3500 | CCAACCATCCATCCACCCATCAA | 4764 | TTGATGGGTGGATGGATGGTTGG |
| 3501 | ACCAACCATCCATCCACCCATCA | 4765 | TGATGGGTGGATGGATGGTTGGT |
| 3502 | AACCAACCATCCATCCACCCATC | 4766 | GATGGGTGGATGGATGGTTGGTT |
| 3503 | CAACCAACCATCCATCCACCCAT | 4767 | ATGGGTGGATGGATGGTTGGTTG |
| 3504 | ACAACCAACCATCCATCCACCCA | 4768 | TGGGTGGATGGATGGTTGGTTGT |
| 3505 | TACAACCAACCATCCATCCACCC | 4769 | GGGTGGATGGATGGTTGGTTGTA |
| 3506 | ATACAACCAACCATCCATCCACC | 4770 | GGTGGATGGATGGTTGGTTGTAT |
| 3507 | CATACAACCAACCATCCATCCAC | 4771 | GTGGATGGATGGTTGGTTGTATG |
| 3508 | TCATACAACCAACCATCCATCCA | 4772 | TGGATGGATGGTTGGTTGTATGA |
| 3509 | TTCATACAACCAACCATCCATCC | 4773 | GGATGGATGGTTGGTTGTATGAA |
| 3510 | TTTCATACAACCAACCATCCATC | 4774 | GATGGATGGTTGGTTGTATGAAA |
| 3511 | CTTTCATACAACCAACCATCCAT | 4775 | ATGGATGGTTGGTTGTATGAAAG |
| 3512 | TCTTTCATACAACCAACCATCCA | 4776 | TGGATGGTTGGTTGTATGAAAGA |
| 3513 | TTCTTTCATACAACCAACCATCC | 4777 | GGATGGTTGGTTGTATGAAAGAA |
| 3514 | ATTCTTTCATACAACCAACCATC | 4778 | GATGGTTGGTTGTATGAAAGAAT |
| 3515 | CATTCTTTCATACAACCAACCAT | 4779 | ATGGTTGGTTGTATGAAAGAATG |
| 3516 | TCATTCTTTCATACAACCAACCA | 4780 | TGGTTGGTTGTATGAAAGAATGA |
| 3517 | TTCATTCTTTCATACAACCAACC | 4781 | GGTTGGTTGTATGAAAGAATGAA |
| 3518 | ATTCATTCTTTCATACAACCAAC | 4782 | GTTGGTTGTATGAAAGAATGAAT |
| 3519 | CATTCATTCTTTCATACAACCAA | 4783 | TTGGTTGTATGAAAGAATGAATG |
| 3520 | TCATTCATTCTTTCATACAACCA | 4784 | TGGTTGTATGAAAGAATGAATGA |
| 3521 | ATCATTCATTCTTTCATACAACC | 4785 | GGTTGTATGAAAGAATGAATGAT |
| 3522 | AATCATTCATTCTTTCATACAAC | 4786 | GTTGTATGAAAGAATGAATGATT |
| 3523 | CAATCATTCATTCTTTCATACAA | 4787 | TTGTATGAAAGAATGAATGATTG |
| 3524 | CCAATCATTCATTCTTTCATACA | 4788 | TGTATGAAAGAATGAATGATTGG |
| 3525 | CCCAATCATTCATTCTTTCATAC | 4789 | GTATGAAAGAATGAATGATTGGG |
| 3526 | ACCCAATCATTCATTCTTTCATA | 4790 | TATGAAAGAATGAATGATTGGGT |
| 3527 | TACCCAATCATTCATTCTTTCAT | 4791 | ATGAAAGAATGAATGATTGGGTA |
| 3528 | CTACCCAATCATTCATTCTTTCA | 4792 | TGAAAGAATGAATGATTGGGTAG |
| 3529 | CCTACCCAATCATTCATTCTTTC | 4793 | GAAAGAATGAATGATTGGGTAGG |
| 3530 | ACCTACCCAATCATTCATTCTTT | 4794 | AAAGAATGAATGATTGGGTAGGT |
| 3531 | CACCTACCCAATCATTCATTCTT | 4795 | AAGAATGAATGATTGGGTAGGTG |
| 3532 | CCACCTACCCAATCATTCATTCT | 4796 | AGAATGAATGATTGGGTAGGTGG |
| 3533 | TCCACCTACCCAATCATTCATTC | 4797 | GAATGAATGATTGGGTAGGTGGA |
| 3534 | ATCCACCTACCCAATCATTCATT | 4798 | AATGAATGATTGGGTAGGTGGAT |
| 3535 | AATCCACCTACCCAATCATTCAT | 4799 | ATGAATGATTGGGTAGGTGGATT |
| 3536 | TAATCCACCTACCCAATCATTCA | 4800 | TGAATGATTGGGTAGGTGGATTA |
| 3537 | TTAATCCACCTACCCAATCATTC | 4801 | GAATGATTGGGTAGGTGGATTAA |
| 3538 | CTTAATCCACCTACCCAATCATT | 4802 | AATGATTGGGTAGGTGGATTAAG |
| 3540 | AACTTAATCCACCTACCCAATCA | 4804 | TGATTGGGTAGGTGGATTAAGTT |
| 3541 | CAACTTAATCCACCTACCCAATC | 4805 | GATTGGGTAGGTGGATTAAGTTG |
| 3542 | GCAACTTAATCCACCTACCCAAT | 4806 | ATTGGGTAGGTGGATTAAGTTGC |
| 3543 | CGCAACTTAATCCACCTACCCAA | 4807 | TTGGGTAGGTGGATTAAGTTGCG |
| 3544 | CCGCAACTTAATCCACCTACCCA | 4808 | TGGGTAGGTGGATTAAGTTGCGG |
| 3545 | TCCGCAACTTAATCCACCTACCC | 4809 | GGGTAGGTGGATTAAGTTGCGGA |
| 3546 | ATCCGCAACTTAATCCACCTACC | 4810 | GGTAGGTGGATTAAGTTGCGGAT |
| 3547 | GATCCGCAACTTAATCCACCTAC | 4811 | GTAGGTGGATTAAGTTGCGGATC |
| 3548 | TGATCCGCAACTTAATCCACCTA | 4812 | TAGGTGGATTAAGTTGCGGATCA |
| 3549 | TTGATCCGCAACTTAATCCACCT | 4813 | AGGTGGATTAAGTTGCGGATCAA |
| 3550 | ATTGATCCGCAACTTAATCCACC | 4814 | GGTGGATTAAGTTGCGGATCAAT |
| 3551 | CATTGATCCGCAACTTAATCCAC | 4815 | GTGGATTAAGTTGCGGATCAATG |
| 3552 | ACATTGATCCGCAACTTAATCCA | 4816 | TGGATTAAGTTGCGGATCAATGT |
| 3553 | TACATTGATCCGCAACTTAATCC | 4817 | GGATTAAGTTGCGGATCAATGTA |
| 3554 | ATACATTGATCCGCAACTTAATC | 4818 | GATTAAGTTGCGGATCAATGTAT |
| 3555 | CATACATTGATCCGCAACTTAAT | 4819 | ATTAAGTTGCGGATCAATGTATG |
| 3556 | CCATACATTGATCCGCAACTTAA | 4820 | TTAAGTTGCGGATCAATGTATGG |
| 3557 | CCCATACATTGATCCGCAACTTA | 4821 | TAAGTTGCGGATCAATGTATGGG |
| 3558 | TCCCATACATTGATCCGCAACTT | 4822 | AAGTTGCGGATCAATGTATGGGA |
| 3559 | ATCCCATACATTGATCCGCAACT | 4823 | AGTTGCGGATCAATGTATGGGAT |
| 3560 | CATCCCATACATTGATCCGCAAC | 4824 | GTTGCGGATCAATGTATGGGATG |
| 3561 | CCATCCCATACATTGATCCGCAA | 4825 | TTGCGGATCAATGTATGGGATGG |
| 3562 | TCCATCCCATACATTGATCCGCA | 4826 | TGCGGATCAATGTATGGGATGGA |
| 3563 | ATCCATCCCATACATTGATCCGC | 4827 | GCGGATCAATGTATGGGATGGAT |
| 3564 | CATCCATCCCATACATTGATCCG | 4828 | CGGATCAATGTATGGGATGGATG |
| 3565 | TCATCCATCCCATACATTGATCC | 4829 | GGATCAATGTATGGGATGGATGA |
| 3566 | TTCATCCATCCCATACATTGATC | 4830 | GATCAATGTATGGGATGGATGAA |
| 3567 | ATTCATCCATCCCATACATTGAT | 4831 | ATCAATGTATGGGATGGATGAAT |
| 3568 | CATTCATCCATCCCATACATTGA | 4832 | TCAATGTATGGGATGGATGAATG |
| 3569 | CCATTCATCCATCCCATACATTG | 4833 | CAATGTATGGGATGGATGAATGG |
| 3570 | TCCATTCATCCATCCCATACATT | 4834 | AATGTATGGGATGGATGAATGGA |
| 3571 | ATCCATTCATCCATCCCATACAT | 4835 | ATGTATGGGATGGATGAATGGAT |
| 3572 | CATCCATTCATCCATCCCATACA | 4836 | TGTATGGGATGGATGAATGGATG |
| 3573 | CCATCCATTCATCCATCCCATAC | 4837 | GTATGGGATGGATGAATGGATGG |
| 3574 | TCCATCCATTCATCCATCCCATA | 4838 | TATGGGATGGATGAATGGATGGA |
| 3575 | ATCCATCCATTCATCCATCCCAT | 4839 | ATGGGATGGATGAATGGATGGAT |
| 3576 | CATCCATCCATTCATCCATCCCA | 4840 | TGGGATGGATGAATGGATGGATG |
| 3577 | CCATCCATCCATTCATCCATCCC | 4841 | GGGATGGATGAATGGATGGATGG |
| 3578 | TCCATCCATCCATTCATCCATCC | 4842 | GGATGGATGAATGGATGGATGGA |
| 3579 | ATCCATCCATCCATTCATCCATC | 4843 | GATGGATGAATGGATGGATGGAT |
| 3580 | CATCCATCCATCCATTCATCCAT | 4844 | ATGGATGAATGGATGGATGGATG |
| 3581 | CCATCCATCCATCCATTCATCCA | 4845 | TGGATGAATGGATGGATGGATGG |
| 3583 | ATCCATCCATCCATCCATTCATC | 4847 | GATGAATGGATGGATGGATGGAT |
| 3584 | CATCCATCCATCCATCCATTCAT | 4848 | ATGAATGGATGGATGGATGGATG |
| 3585 | ACATCCATCCATCCATCCATTCA | 4849 | TGAATGGATGGATGGATGGATGT |
| 3586 | CACATCCATCCATCCATCCATTC | 4850 | GAATGGATGGATGGATGGATGTG |
| 3587 | ACACATCCATCCATCCATCCATT | 4851 | AATGGATGGATGGATGGATGTGT |
| 3588 | CACACATCCATCCATCCATCCAT | 4852 | ATGGATGGATGGATGGATGTGTG |
| 3589 | CCACACATCCATCCATCCATCCA | 4853 | TGGATGGATGGATGGATGTGTGG |
| 3590 | ACCACACATCCATCCATCCATCC | 4854 | GGATGGATGGATGGATGTGTGGT |
| 3591 | AACCACACATCCATCCATCCATC | 4855 | GATGGATGGATGGATGTGTGGTT |
| 3592 | CAACCACACATCCATCCATCCAT | 4856 | ATGGATGGATGGATGTGTGGTTG |
| 3593 | TCAACCACACATCCATCCATCCA | 4857 | TGGATGGATGGATGTGTGGTTGA |
| 3594 | TTCAACCACACATCCATCCATCC | 4858 | GGATGGATGGATGTGTGGTTGAA |
| 3595 | ATTCAACCACACATCCATCCATC | 4859 | GATGGATGGATGTGTGGTTGAAT |
| 3596 | AATTCAACCACACATCCATCCAT | 4860 | ATGGATGGATGTGTGGTTGAATT |
| 3597 | TAATTCAACCACACATCCATCCA | 4861 | TGGATGGATGTGTGGTTGAATTA |
| 3598 | GTAATTCAACCACACATCCATCC | 4862 | GGATGGATGTGTGGTTGAATTAC |
| 3599 | AGTAATTCAACCACACATCCATC | 4863 | GATGGATGTGTGGTTGAATTACT |
| 3600 | CAGTAATTCAACCACACATCCAT | 4864 | ATGGATGTGTGGTTGAATTACTG |
| 3601 | TCAGTAATTCAACCACACATCCA | 4865 | TGGATGTGTGGTTGAATTACTGA |
| 3602 | TTCAGTAATTCAACCACACATCC | 4866 | GGATGTGTGGTTGAATTACTGAA |
| 3603 | TTTCAGTAATTCAACCACACATC | 4867 | GATGTGTGGTTGAATTACTGAAA |
| 3604 | CTTTCAGTAATTCAACCACACAT | 4868 | ATGTGTGGTTGAATTACTGAAAG |
| 3605 | CCTTTCAGTAATTCAACCACACA | 4869 | TGTGTGGTTGAATTACTGAAAGG |
| 3606 | ACCTTTCAGTAATTCAACCACAC | 4870 | GTGTGGTTGAATTACTGAAAGGT |
| 3607 | AACCTTTCAGTAATTCAACCACA | 4871 | TGTGGTTGAATTACTGAAAGGTT |
| 3608 | CAACCTTTCAGTAATTCAACCAC | 4872 | GTGGTTGAATTACTGAAAGGTTG |
| 3609 | CCAACCTTTCAGTAATTCAACCA | 4873 | TGGTTGAATTACTGAAAGGTTGG |
| 3610 | TCCAACCTTTCAGTAATTCAACC | 4874 | GGTTGAATTACTGAAAGGTTGGA |
| 3611 | TTCCAACCTTTCAGTAATTCAAC | 4875 | GTTGAATTACTGAAAGGTTGGAA |
| 3612 | CTTCCAACCTTTCAGTAATTCAA | 4876 | TTGAATTACTGAAAGGTTGGAAG |
| 3613 | TCTTCCAACCTTTCAGTAATTCA | 4877 | TGAATTACTGAAAGGTTGGAAGA |
| 3614 | CTCTTCCAACCTTTCAGTAATTC | 4878 | GAATTACTGAAAGGTTGGAAGAG |
| 3615 | ACTCTTCCAACCTTTCAGTAATT | 4879 | AATTACTGAAAGGTTGGAAGAGT |
| 3616 | CACTCTTCCAACCTTTCAGTAAT | 4880 | ATTACTGAAAGGTTGGAAGAGTG |
| 3617 | CCACTCTTCCAACCTTTCAGTAA | 4881 | TTACTGAAAGGTTGGAAGAGTGG |
| 3618 | TCCACTCTTCCAACCTTTCAGTA | 4882 | TACTGAAAGGTTGGAAGAGTGGA |
| 3619 | ATCCACTCTTCCAACCTTTCAGT | 4883 | ACTGAAAGGTTGGAAGAGTGGAT |
| 3620 | CATCCACTCTTCCAACCTTTCAG | 4884 | CTGAAAGGTTGGAAGAGTGGATG |
| 3621 | CCATCCACTCTTCCAACCTTTCA | 4885 | TGAAAGGTTGGAAGAGTGGATGG |
| 3622 | CCCATCCACTCTTCCAACCTTTC | 4886 | GAAAGGTTGGAAGAGTGGATGGG |
| 3623 | ACCCATCCACTCTTCCAACCTTT | 4887 | AAAGGTTGGAAGAGTGGATGGGT |
| 3624 | CACCCATCCACTCTTCCAACCTT | 4888 | AAGGTTGGAAGAGTGGATGGGTG |
| 3626 | TTCACCCATCCACTCTTCCAACC | 4890 | GGTTGGAAGAGTGGATGGGTGAA |
| 3627 | TTTCACCCATCCACTCTTCCAAC | 4891 | GTTGGAAGAGTGGATGGGTGAAA |
| 3628 | ATTTCACCCATCCACTCTTCCAA | 4892 | TTGGAAGAGTGGATGGGTGAAAT |
| 3629 | AATTTCACCCATCCACTCTTCCA | 4893 | TGGAAGAGTGGATGGGTGAAATT |
| 3630 | AAATTTCACCCATCCACTCTTCC | 4894 | GGAAGAGTGGATGGGTGAAATTT |
| 3631 | CAAATTTCACCCATCCACTCTTC | 4895 | GAAGAGTGGATGGGTGAAATTTG |
| 3632 | CCAAATTTCACCCATCCACTCTT | 4896 | AAGAGTGGATGGGTGAAATTTGG |
| 3633 | CCCAAATTTCACCCATCCACTCT | 4897 | AGAGTGGATGGGTGAAATTTGGG |
| 3634 | CCCCAAATTTCACCCATCCACTC | 4898 | GAGTGGATGGGTGAAATTTGGGG |
| 3635 | ACCCCAAATTTCACCCATCCACT | 4899 | AGTGGATGGGTGAAATTTGGGGT |
| 3636 | TACCCCAAATTTCACCCATCCAC | 4900 | GTGGATGGGTGAAATTTGGGGTA |
| 3637 | CTACCCCAAATTTCACCCATCCA | 4901 | TGGATGGGTGAAATTTGGGGTAG |
| 3638 | ACTACCCCAAATTTCACCCATCC | 4902 | GGATGGGTGAAATTTGGGGTAGT |
| 3639 | AACTACCCCAAATTTCACCCATC | 4903 | GATGGGTGAAATTTGGGGTAGTT |
| 3640 | TAACTACCCCAAATTTCACCCAT | 4904 | ATGGGTGAAATTTGGGGTAGTTA |
| 3641 | CTAACTACCCCAAATTTCACCCA | 4905 | TGGGTGAAATTTGGGGTAGTTAG |
| 3642 | TCTAACTACCCCAAATTTCACCC | 4906 | GGGTGAAATTTGGGGTAGTTAGA |
| 3643 | ATCTAACTACCCCAAATTTCACC | 4907 | GGTGAAATTTGGGGTAGTTAGAT |
| 3644 | CATCTAACTACCCCAAATTTCAC | 4908 | GTGAAATTTGGGGTAGTTAGATG |
| 3645 | CCATCTAACTACCCCAAATTTCA | 4909 | TGAAATTTGGGGTAGTTAGATGG |
| 3646 | CCCATCTAACTACCCCAAATTTC | 4910 | GAAATTTGGGGTAGTTAGATGGG |
| 3647 | ACCCATCTAACTACCCCAAATTT | 4911 | AAATTTGGGGTAGTTAGATGGGT |
| 3648 | CACCCATCTAACTACCCCAAATT | 4912 | AATTTGGGGTAGTTAGATGGGTG |
| 3649 | CCACCCATCTAACTACCCCAAAT | 4913 | ATTTGGGGTAGTTAGATGGGTGG |
| 3650 | CCCACCCATCTAACTACCCCAAA | 4914 | TTTGGGGTAGTTAGATGGGTGGG |
| 3651 | ACCCACCCATCTAACTACCCCAA | 4915 | TTGGGGTAGTTAGATGGGTGGGT |
| 3652 | CACCCACCCATCTAACTACCCCA | 4916 | TGGGGTAGTTAGATGGGTGGGTG |
| 3653 | ACACCCACCCATCTAACTACCCC | 4917 | GGGGTAGTTAGATGGGTGGGTGT |
| 3654 | CACACCCACCCATCTAACTACCC | 4918 | GGGTAGTTAGATGGGTGGGTGTG |
| 3655 | ACACACCCACCCATCTAACTACC | 4919 | GGTAGTTAGATGGGTGGGTGTGT |
| 3656 | CACACACCCACCCATCTAACTAC | 4920 | GTAGTTAGATGGGTGGGTGTGTG |
| 3657 | CCACACACCCACCCATCTAACTA | 4921 | TAGTTAGATGGGTGGGTGTGTGG |
| 3658 | TCCACACACCCACCCATCTAACT | 4922 | AGTTAGATGGGTGGGTGTGTGGA |
| 3659 | ATCCACACACCCACCCATCTAAC | 4923 | GTTAGATGGGTGGGTGTGTGGAT |
| 3660 | CATCCACACACCCACCCATCTAA | 4924 | TTAGATGGGTGGGTGTGTGGATG |
| 3661 | CCATCCACACACCCACCCATCTA | 4925 | TAGATGGGTGGGTGTGTGGATGG |
| 3662 | TCCATCCACACACCCACCCATCT | 4926 | AGATGGGTGGGTGTGTGGATGGA |
| 3663 | ATCCATCCACACACCCACCCATC | 4927 | GATGGGTGGGTGTGTGGATGGAT |
| 3664 | TATCCATCCACACACCCACCCAT | 4928 | ATGGGTGGGTGTGTGGATGGATA |
| 3665 | TTATCCATCCACACACCCACCCA | 4929 | TGGGTGGGTGTGTGGATGGATAA |
| 3666 | TTTATCCATCCACACACCCACCC | 4930 | GGGTGGGTGTGTGGATGGATAAA |
| 3667 | TTTTATCCATCCACACACCCACC | 4931 | GGTGGGTGTGTGGATGGATAAAA |
| 3669 | TCTTTTATCCATCCACACACCCA | 4933 | TGGGTGTGTGGATGGATAAAAGA |
| 3670 | CTCTTTTATCCATCCACACACCC | 4934 | GGGTGTGTGGATGGATAAAAGAG |
| 3671 | ACTCTTTTATCCATCCACACACC | 4935 | GGTGTGTGGATGGATAAAAGAGT |
| 3672 | TACTCTTTTATCCATCCACACAC | 4936 | GTGTGTGGATGGATAAAAGAGTA |
| 3673 | CTACTCTTTTATCCATCCACACA | 4937 | TGTGTGGATGGATAAAAGAGTAG |
| 3674 | TCTACTCTTTTATCCATCCACAC | 4938 | GTGTGGATGGATAAAAGAGTAGA |
| 3675 | ATCTACTCTTTTATCCATCCACA | 4939 | TGTGGATGGATAAAAGAGTAGAT |
| 3676 | CATCTACTCTTTTATCCATCCAC | 4940 | GTGGATGGATAAAAGAGTAGATG |
| 3677 | TCATCTACTCTTTTATCCATCCA | 4941 | TGGATGGATAAAAGAGTAGATGA |
| 3678 | TTCATCTACTCTTTTATCCATCC | 4942 | GGATGGATAAAAGAGTAGATGAA |
| 3679 | ATTCATCTACTCTTTTATCCATC | 4943 | GATGGATAAAAGAGTAGATGAAT |
| 3680 | CATTCATCTACTCTTTTATCCAT | 4944 | ATGGATAAAAGAGTAGATGAATG |
| 3681 | TCATTCATCTACTCTTTTATCCA | 4945 | TGGATAAAAGAGTAGATGAATGA |
| 3682 | TTCATTCATCTACTCTTTTATCC | 4946 | GGATAAAAGAGTAGATGAATGAA |
| 3683 | ATTCATTCATCTACTCTTTTATC | 4947 | GATAAAAGAGTAGATGAATGAAT |
| 3684 | AATTCATTCATCTACTCTTTTAT | 4948 | ATAAAAGAGTAGATGAATGAATT |
| 3685 | TAATTCATTCATCTACTCTTTTA | 4949 | TAAAAGAGTAGATGAATGAATTA |
| 3686 | TTAATTCATTCATCTACTCTTTT | 4950 | AAAAGAGTAGATGAATGAATTAA |
| 3687 | ATTAATTCATTCATCTACTCTTT | 4951 | AAAGAGTAGATGAATGAATTAAT |
| 3688 | CATTAATTCATTCATCTACTCTT | 4952 | AAGAGTAGATGAATGAATTAATG |
| 3689 | TCATTAATTCATTCATCTACTCT | 4953 | AGAGTAGATGAATGAATTAATGA |
| 3690 | TTCATTAATTCATTCATCTACTC | 4954 | GAGTAGATGAATGAATTAATGAA |
| 3691 | ATTCATTAATTCATTCATCTACT | 4955 | AGTAGATGAATGAATTAATGAAT |
| 3692 | TATTCATTAATTCATTCATCTAC | 4956 | GTAGATGAATGAATTAATGAATA |
| 3693 | TTATTCATTAATTCATTCATCTA | 4957 | TAGATGAATGAATTAATGAATAA |
| 3694 | TTTATTCATTAATTCATTCATCT | 4958 | AGATGAATGAATTAATGAATAAA |
| 3695 | GTTTATTCATTAATTCATTCATC | 4959 | GATGAATGAATTAATGAATAAAC |
| 3696 | TGTTTATTCATTAATTCATTCAT | 4960 | ATGAATGAATTAATGAATAAACA |
| 3697 | CTGTTTATTCATTAATTCATTCA | 4961 | TGAATGAATTAATGAATAAACAG |
| 3698 | CCTGTTTATTCATTAATTCATTC | 4962 | GAATGAATTAATGAATAAACAGG |
| 3699 | GCCTGTTTATTCATTAATTCATT | 4963 | AATGAATTAATGAATAAACAGGC |
| 3700 | TGCCTGTTTATTCATTAATTCAT | 4964 | ATGAATTAATGAATAAACAGGCA |
| 3701 | CTGCCTGTTTATTCATTAATTCA | 4965 | TGAATTAATGAATAAACAGGCAG |
| 3702 | TCTGCCTGTTTATTCATTAATTC | 4966 | GAATTAATGAATAAACAGGCAGA |
| 3703 | ATCTGCCTGTTTATTCATTAATT | 4967 | AATTAATGAATAAACAGGCAGAT |
| 3704 | CATCTGCCTGTTTATTCATTAAT | 4968 | ATTAATGAATAAACAGGCAGATG |
| 3705 | CCATCTGCCTGTTTATTCATTAA | 4969 | TTAATGAATAAACAGGCAGATGG |
| 3706 | TCCATCTGCCTGTTTATTCATTA | 4970 | TAATGAATAAACAGGCAGATGGA |
| 3707 | ATCCATCTGCCTGTTTATTCATT | 4971 | AATGAATAAACAGGCAGATGGAT |
| 3708 | CATCCATCTGCCTGTTTATTCAT | 4972 | ATGAATAAACAGGCAGATGGATG |
| 3709 | TCATCCATCTGCCTGTTTATTCA | 4973 | TGAATAAACAGGCAGATGGATGA |
| 3710 | ATCATCCATCTGCCTGTTTATTC | 4974 | GAATAAACAGGCAGATGGATGAT |
| 3712 | ACATCATCCATCTGCCTGTTTAT | 4976 | ATAAACAGGCAGATGGATGATGT |
| 3713 | TACATCATCCATCTGCCTGTTTA | 4977 | TAAACAGGCAGATGGATGATGTA |
| 3714 | TTACATCATCCATCTGCCTGTTT | 4978 | AAACAGGCAGATGGATGATGTAA |
| 3715 | CTTACATCATCCATCTGCCTGTT | 4979 | AACAGGCAGATGGATGATGTAAG |
| 3716 | GCTTACATCATCCATCTGCCTGT | 4980 | ACAGGCAGATGGATGATGTAAGC |
| 3717 | AGCTTACATCATCCATCTGCCTG | 4981 | CAGGCAGATGGATGATGTAAGCT |
| 3718 | CAGCTTACATCATCCATCTGCCT | 4982 | AGGCAGATGGATGATGTAAGCTG |
| 3719 | GCAGCTTACATCATCCATCTGCC | 4983 | GGCAGATGGATGATGTAAGCTGC |
| 3720 | GGCAGCTTACATCATCCATCTGC | 4984 | GCAGATGGATGATGTAAGCTGCC |
| 3721 | GGGCAGCTTACATCATCCATCTG | 4985 | CAGATGGATGATGTAAGCTGCCC |
| 3722 | GGGGCAGCTTACATCATCCATCT | 4986 | AGATGGATGATGTAAGCTGCCCC |
| 3723 | TGGGGCAGCTTACATCATCCATC | 4987 | GATGGATGATGTAAGCTGCCCCA |
| 3724 | CTGGGGCAGCTTACATCATCCAT | 4988 | ATGGATGATGTAAGCTGCCCCAG |
| 3725 | TCTGGGGCAGCTTACATCATCCA | 4989 | TGGATGATGTAAGCTGCCCCAGA |
| 3726 | GTCTGGGGCAGCTTACATCATCC | 4990 | GGATGATGTAAGCTGCCCCAGAC |
| 3727 | GGTCTGGGGCAGCTTACATCATC | 4991 | GATGATGTAAGCTGCCCCAGACC |
| 3728 | GGGTCTGGGGCAGCTTACATCAT | 4992 | ATGATGTAAGCTGCCCCAGACCC |
| 3729 | AGGGTCTGGGGCAGCTTACATCA | 4993 | TGATGTAAGCTGCCCCAGACCCT |
| 3730 | CAGGGTCTGGGGCAGCTTACATC | 4994 | GATGTAAGCTGCCCCAGACCCTG |
| 3731 | CCAGGGTCTGGGGCAGCTTACAT | 4995 | ATGTAAGCTGCCCCAGACCCTGG |
| 3732 | CCCAGGGTCTGGGGCAGCTTACA | 4996 | TGTAAGCTGCCCCAGACCCTGGG |
| 3733 | TCCCAGGGTCTGGGGCAGCTTAC | 4997 | GTAAGCTGCCCCAGACCCTGGGA |
| 3734 | GTCCCAGGGTCTGGGGCAGCTTA | 4998 | TAAGCTGCCCCAGACCCTGGGAC |
| 3735 | GGTCCCAGGGTCTGGGGCAGCTT | 4999 | AAGCTGCCCCAGACCCTGGGACC |
| 3736 | AGGTCCCAGGGTCTGGGGCAGCT | 5000 | AGCTGCCCCAGACCCTGGGACCT |
| 3737 | GAGGTCCCAGGGTCTGGGGCAGC | 5001 | GCTGCCCCAGACCCTGGGACCTC |
| 3738 | AGAGGTCCCAGGGTCTGGGGCAG | 5002 | CTGCCCCAGACCCTGGGACCTCT |
| 3739 | CAGAGGTCCCAGGGTCTGGGGCA | 5003 | TGCCCCAGACCCTGGGACCTCTG |
| 3740 | TCAGAGGTCCCAGGGTCTGGGGC | 5004 | GCCCCAGACCCTGGGACCTCTGA |
| 3741 | GTCAGAGGTCCCAGGGTCTGGGG | 5005 | CCCCAGACCCTGGGACCTCTGAC |
| 3742 | GGTCAGAGGTCCCAGGGTCTGGG | 5006 | CCCAGACCCTGGGACCTCTGACC |
| 3743 | GGGTCAGAGGTCCCAGGGTCTGG | 5007 | CCAGACCCTGGGACCTCTGACCC |
| 3744 | GGGGTCAGAGGTCCCAGGGTCTG | 5008 | CAGACCCTGGGACCTCTGACCCC |
| 3745 | GGGGGTCAGAGGTCCCAGGGTCT | 5009 | AGACCCTGGGACCTCTGACCCCC |
| 3746 | CGGGGGTCAGAGGTCCCAGGGTC | 5010 | GACCCTGGGACCTCTGACCCCCG |
| 3747 | CCGGGGGTCAGAGGTCCCAGGGT | 5011 | ACCCTGGGACCTCTGACCCCCGG |
| 3748 | GCCGGGGGTCAGAGGTCCCAGGG | 5012 | CCCTGGGACCTCTGACCCCCGGC |
| 3749 | CGCCGGGGGTCAGAGGTCCCAGG | 5013 | CCTGGGACCTCTGACCCCCGGCG |
| 3750 | TCGCCGGGGGTCAGAGGTCCCAG | 5014 | CTGGGACCTCTGACCCCCGGCGA |
| 3751 | GTCGCCGGGGGTCAGAGGTCCCA | 5015 | TGGGACCTCTGACCCCCGGCGAC |
| 3752 | GGTCGCCGGGGGTCAGAGGTCCC | 5016 | GGGACCTCTGACCCCCGGCGACC |
| 3753 | GGGTCGCCGGGGGTCAGAGGTCC | 5017 | GGACCTCTGACCCCCGGCGACCC |
| 3755 | AGGGGTCGCCGGGGGTCAGAGGT | 5019 | ACCTCTGACCCCCGGCGACCCCT |
| 3756 | AAGGGGTCGCCGGGGGTCAGAGG | 5020 | CCTCTGACCCCCGGCGACCCCTT |
| 3757 | CAAGGGGTCGCCGGGGGTCAGAG | 5021 | CTCTGACCCCCGGCGACCCCTTG |
| 3758 | GCAAGGGGTCGCCGGGGGTCAGA | 5022 | TCTGACCCCCGGCGACCCCTTGC |
| 3759 | TGCAAGGGGTCGCCGGGGGTCAG | 5023 | CTGACCCCCGGCGACCCCTTGCA |
| 3760 | GTGCAAGGGGTCGCCGGGGGTCA | 5024 | TGACCCCCGGCGACCCCTTGCAC |
| 3761 | AGTGCAAGGGGTCGCCGGGGGTC | 5025 | GACCCCCGGCGACCCCTTGCACT |
| 3762 | GAGTGCAAGGGGTCGCCGGGGGT | 5026 | ACCCCCGGCGACCCCTTGCACTC |
| 3763 | AGAGTGCAAGGGGTCGCCGGGGG | 5027 | CCCCCGGCGACCCCTTGCACTCT |
| 3764 | GAGAGTGCAAGGGGTCGCCGGGG | 5028 | CCCCGGCGACCCCTTGCACTCTC |
| 3765 | GGAGAGTGCAAGGGGTCGCCGGG | 5029 | CCCGGCGACCCCTTGCACTCTCC |
| 3766 | TGGAGAGTGCAAGGGGTCGCCGG | 5030 | CCGGCGACCCCTTGCACTCTCCA |
| 3767 | ATGGAGAGTGCAAGGGGTCGCCG | 5031 | CGGCGACCCCTTGCACTCTCCAT |
| 3768 | CATGGAGAGTGCAAGGGGTCGCC | 5032 | GGCGACCCCTTGCACTCTCCATG |
| 3769 | TCATGGAGAGTGCAAGGGGTCGC | 5033 | GCGACCCCTTGCACTCTCCATGA |
| 3770 | GTCATGGAGAGTGCAAGGGGTCG | 5034 | CGACCCCTTGCACTCTCCATGAC |
| 3771 | TGTCATGGAGAGTGCAAGGGGTC | 5035 | GACCCCTTGCACTCTCCATGACA |
| 3772 | GTGTCATGGAGAGTGCAAGGGGT | 5036 | ACCCCTTGCACTCTCCATGACAC |
| 3773 | AGTGTCATGGAGAGTGCAAGGGG | 5037 | CCCCTTGCACTCTCCATGACACT |
| 3774 | AAGTGTCATGGAGAGTGCAAGGG | 5038 | CCCTTGCACTCTCCATGACACTT |
| 3775 | AAAGTGTCATGGAGAGTGCAAGG | 5039 | CCTTGCACTCTCCATGACACTTT |
| 3776 | GAAAGTGTCATGGAGAGTGCAAG | 5040 | CTTGCACTCTCCATGACACTTTC |
| 3777 | AGAAAGTGTCATGGAGAGTGCAA | 5041 | TTGCACTCTCCATGACACTTTCT |
| 3778 | GAGAAAGTGTCATGGAGAGTGCA | 5042 | TGCACTCTCCATGACACTTTCTC |
| 3779 | AGAGAAAGTGTCATGGAGAGTGC | 5043 | GCACTCTCCATGACACTTTCTCT |
| 3780 | GAGAGAAAGTGTCATGGAGAGTG | 5044 | CACTCTCCATGACACTTTCTCTC |
| 3781 | GGAGAGAAAGTGTCATGGAGAGT | 5045 | ACTCTCCATGACACTTTCTCTCC |
| 3782 | GGGAGAGAAAGTGTCATGGAGAG | 5046 | CTCTCCATGACACTTTCTCTCCC |
| 3783 | TGGGAGAGAAAGTGTCATGGAGA | 5047 | TCTCCATGACACTTTCTCTCCCA |
| 3784 | ATGGGAGAGAAAGTGTCATGGAG | 5048 | CTCCATGACACTTTCTCTCCCAT |
| 3785 | CATGGGAGAGAAAGTGTCATGGA | 5049 | TCCATGACACTTTCTCTCCCATG |
| 3786 | CCATGGGAGAGAAAGTGTCATGG | 5050 | CCATGACACTTTCTCTCCCATGG |
| 3787 | ACCATGGGAGAGAAAGTGTCATG | 5051 | CATGACACTTTCTCTCCCATGGT |
| 3788 | CACCATGGGAGAGAAAGTGTCAT | 5052 | ATGACACTTTCTCTCCCATGGTG |
| 3789 | CCACCATGGGAGAGAAAGTGTCA | 5053 | TGACACTTTCTCTCCCATGGTGG |
| 3790 | GCCACCATGGGAGAGAAAGTGTC | 5054 | GACACTTTCTCTCCCATGGTGGC |
| 3791 | TGCCACCATGGGAGAGAAAGTGT | 5055 | ACACTTTCTCTCCCATGGTGGCA |
| 3792 | CTGCCACCATGGGAGAGAAAGTG | 5056 | CACTTTCTCTCCCATGGTGGCAG |

*At least one nucleoside linkage of the nucleobase sequence is selected from a
phosphorothioate linkage, an alkyl phosphate linkage, a phosphorodithioate linkage,
a phosphotriester linkage, an alkylphosphonate linkage, a 3-methoxypropyl phosphonate
linkage, a methylphosphonate linkage, an aminoalkylphosphotriester linkage, an
alkylene phosphonate linkage, a phosphinate linkage, a phosphoramidate linkage, a
phosphoramidothioate linkage, a thiophosphorodiamidate linkage, a phosphorodiamidate
(e.g., comprising a phosphorodiamidate morpholino (PMO), 3′amino ribose, or 5′ amino
ribose) linkage, an aminoalkylphosphoramidate linkage, a thiophosphoramidate linkage,
a thionoalkylphosphonate linkage, a thionoalkylphosphotriester linkage, a
thiophosphate linkage, a selenophosphate linkage, and a boranophosphate linkage.

TABLE 2B
Example UNC13A Oligonucleotide
Variant Sequences

| SEQ | | SEQ | |
|---|---|---|---|
| ID | AON Sequence* | ID | Target Sequence |
| NO: | (5′ → 3′) | NO: | (5′ → 3′) |
| 5209 | TCTTTCCAGGAAACCCAG | 5222 | TGCCTGGGTTTCCTGGAA |
| | GCA | | AGA |
| 5210 | CTTTCCAGGAAACCCAGG | 5223 | TGCCTGGGTTTCCTGGAA |
| | CA | | AG |
| 5211 | TCTTTCCAGGAAACCCAG | 5224 | GCCTGGGTTTCCTGGAAA |
| | GC | | GA |
| 5212 | TTTCCAGGAAACCCAGGC | 5225 | TGCCTGGGTTTCCTGGA |
| | A | | AA |
| 5213 | TCTTTCCAGGAAACCCAG | 5226 | CCTGGGTTTCCTGGAAA |
| | G | | GA |
| 5214 | TTCCAGGAAACCCAGGCA | 5227 | TGCCTGGGTTTCCTGGAA |
| 5215 | TCTTTCCAGGAAACCCAG | 5228 | CTGGGTTTCCTGGAAAGA |
| 5216 | TCCAGGAAACCCAGGCA | 5229 | TGCCTGGGTTTCCTGGA |
| 5217 | TCTTTCCAGGAAACCCA | 5230 | TGGGTTTCCTGGAAAGA |
| 5218 | CCAGGAAACCCAGGCA | 5231 | TGCCTGGGTTTCCTGG |
| 5219 | TCTTTCCAGGAAACCC | 5232 | GGGTTTCCTGGAAAGA |
| 5220 | CAGGAAACCCAGGCA | 5233 | TGCCTGGGTTTCCTG |
| 5221 | TCTTTCCAGGAAACC | 5234 | GGTTTCCTGGAAAGA |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, each of the nucleosides of antisense oligonucleotides shown in Table 2B are modified nucleosides with 2′-O-(2-methoxyethyl) (2′MOE) sugar moieties, and each “C” is replaced with a 5-methylcytosine (5-MeC). Additionally, each of the internucleoside linkages between the nucleosides are phosphorothioate internucleoside linkages.

Antisense Oligonucleotides with One or More Locked Nucleic Acids (LNAs)

In various embodiments, antisense oligonucleotides disclosed herein (e.g., UNC13A parent oligonucleotides and/or UNC13A oligonucleotide variants) comprise one or more locked nucleic acids (LNAs). In particular embodiments, an antisense oligonucleotide includes one LNA. In particular embodiments, an antisense oligonucleotide includes two LNAs. In particular embodiments, an antisense oligonucleotide includes three LNAs. Generally, a LNA refers to nucleic acid monomers having a bridge (e.g., methylene, ethylene, aminooxy, or oxyimino bridge) connecting two carbon atoms between the 4′ and 2′ position of the nucleoside sugar unit, thereby forming a bicyclic sugar.

In some embodiments, an antisense oligonucleotide disclosed herein includes, if counting from 5′ to 3′, a LNA at a 4^{th} position of the antisense oligonucleotide. In some embodiments, an antisense oligonucleotide disclosed herein includes, if counting from 5′ to 3′, a LNA at a 7^{th} position of the antisense oligonucleotide. In some embodiments, an antisense oligonucleotide disclosed herein includes, if counting from 5′ to 3′, a LNA at a 9^{th} position of the antisense oligonucleotide. In some embodiments, an antisense oligonucleotide disclosed herein includes, if counting from 5′ to 3′, a LNA at a 12^{th} position of the antisense oligonucleotide. In some embodiments, an antisense oligonucleotide disclosed herein includes, if counting from 5′ to 3′, a LNA at a 15^{th} position of the antisense oligonucleotide. In some embodiments, an antisense oligonucleotide disclosed herein includes, if counting from 5′ to 3′, a LNA at a 17^{th} position of the antisense oligonucleotide. In some embodiments, an antisense oligonucleotide disclosed herein includes, if counting from 5′ to 3′, a LNA at a 20^{th} position of the antisense oligonucleotide.

In various embodiments, antisense oligonucleotides disclosed herein (e.g., UNC13A parent oligonucleotides and/or UNC13A oligonucleotide variants) comprise two LNAs located at two different positions of the antisense oligonucleotide. In some embodiments, an antisense oligonucleotide disclosed herein includes, if counting from 5′ to 3′, a first LNA at a fourth position of the antisense oligonucleotide and a second LNA at a 20^{th} position of the antisense oligonucleotide. In some embodiments, an antisense oligonucleotide disclosed herein includes, if counting from 5′ to 3′, a first LNA at a 7^{th} position of the antisense oligonucleotide and a second LNA at a 15^{th} position of the antisense oligonucleotide. In some embodiments, an antisense oligonucleotide disclosed herein includes, if counting from 5′ to 3′, a first LNA at a 7^{th} position of the antisense oligonucleotide and a second LNA at a 17^{th} position of the antisense oligonucleotide. In some embodiments, an antisense oligonucleotide disclosed herein includes, if counting from 5′ to 3′, a first LNA at a 9^{th} position of the antisense oligonucleotide and a second LNA at a 17^{th} position of the antisense oligonucleotide.

In various embodiments, antisense oligonucleotides disclosed herein (e.g., UNC13A parent oligonucleotides and/or UNC13A oligonucleotide variants) comprise three LNAs located at three different positions of the antisense oligonucleotide. In some embodiments, an antisense oligonucleotide disclosed herein includes, if counting from 5′ to 3′, a first LNA at a 4^{th} position of the antisense oligonucleotide, a second LNA at a 12^{th} position of the antisense oligonucleotide, and a third LNA at a 20^{th} position of the antisense oligonucleotide.

Antisense Oligonucleotides with One or More Spacers

In various embodiments, antisense oligonucleotides comprise one or more spacers. In particular embodiments, an antisense oligonucleotide includes one spacer. In particular embodiments, an antisense oligonucleotide includes two spacers. In particular embodiments, an antisense oligonucleotide includes three spacers. Generally, a spacer refers to a nucleoside-replacement group lacking a nucleotide base and wherein the nucleoside sugar moiety is replaced by a non-sugar substitute group. The non-sugar substitute group is not capable of linking to a nucleobase, but is capable of linking with the 3′ and 5′ positions of nucleosides adjacent to the spacer through an internucleoside linking group.

In certain embodiments, an oligonucleotide with one or more spacers, such as disclosed herein, may be an oligonucleotide with 5 to 100 oligonucleotide units in length, for example, 10 to 60 oligonucleotide units in length, for example, 12 to 50 oligonucleotide units in length, 14 to 40 oligonucleotide units in length, 10 to 30 oligonucleotide units in length, for example, 14 to 30 oligonucleotide units in length, for example, 14 to 25 or 15 to 22 oligonucleotide units in length, or 18, 19, 20, 21, 22, 23, 24, or 25 oligonucleotide units in length. As used herein, an “oligonucleotide unit” refers to either a nucleoside (e.g., a nucleoside which includes a sugar and/or a nucleobase) or a nucleoside-replacement group (e.g., a spacer) of the oligonucleotide.

In particular embodiments, oligonucleotides with one or more spacers are 25 oligonucleotide units in length. In particular embodiments, the oligonucleotides with one or more spacers are 23 oligonucleotide units in length. In particular embodiments, the oligonucleotides with one or more spacers are 21 oligonucleotide units in length. In particular embodiments, the oligonucleotides with one or more spacers are 19 oligonucleotide units in length. In various embodiments, the oligonucleotides with one or more spacers are at least 18, at least 19, at least 20, at least 21, at least 22, at least 23, at least 24, or at least 25 oligonucleotide units in length. In various embodiments, the oligonucleotides with one or more spacers are at least 18 oligonucleotide units in length. In various embodiments, the oligonucleotides with one or more spacers are at least 19 oligonucleotide units in length. In various embodiments, the oligonucleotides with one or more spacers are at least 20 oligonucleotide units in length. In various embodiments, the oligonucleotides with one or more spacers are at least 21 oligonucleotide units in length. In various embodiments, the oligonucleotides with one or more spacers are at least 22 oligonucleotide units in length. In various embodiments, the oligonucleotides with one or more spacers are at least 23 oligonucleotide units in length. In various embodiments, the oligonucleotides with one or more spacers are at least 24 oligonucleotide units in length. In various embodiments, the oligonucleotides with one or more spacers are at least 25 oligonucleotide units in length.

In various embodiments, a UNC13A AON comprises a sequence that shares at least 80% identity with an equal length portion of any one of SEQ ID NOs: 5066-5166, SEQ ID NOs: 5168-5202, SEQ ID NOs: 5209-5221, or SEQ ID NOs: 5235-5292. In various embodiments, a UNC13A AON comprises a sequence that shares at least 85% identity with an equal length portion of any one of SEQ ID NOs: 5066-5166, SEQ ID NOs: 5168-5202, SEQ ID NOs: 5209-5221, or SEQ ID NOs: 5235-5292. In various embodiments, a UNC13A AON comprises a sequence that shares at least 90% identity with an equal length portion of any one of SEQ ID NOs: 5066-5166, SEQ ID NOs: 5168-5202, SEQ ID NOs: 5209-5221, or SEQ ID NOs: 5235-5292. In various embodiments, a UNC13A AON comprises a sequence that shares at least 95% identity with an equal length portion of any one of SEQ ID NOs: 5066-5166, SEQ ID NOs: 5168-5202, SEQ ID NOs: 5209-5221, or SEQ ID NOs: 5235-5292. In various embodiments, a UNC13A AON comprises a sequence that shares 100% identity with an equal length portion of any one of SEQ ID NOs: 5066-5166, SEQ ID NOs: 5168-5202, SEQ ID NOs: 5209-5221, or SEQ ID NOs: 5235-5292.

In some embodiments, the spacer is of Formula (X):

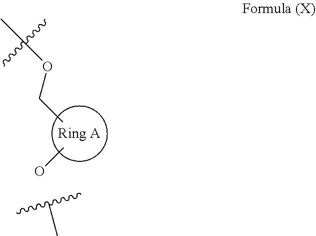

wherein ring A is as defined herein.

In some embodiments, the spacer is of Formula (Xa):

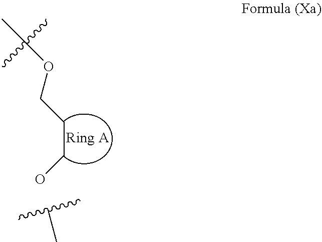

wherein ring A is as defined herein and the —CH₂—O— group is on a ring A atom adjacent to the —O— group.

As generally defined herein, ring A of formulae (X) and (Xa), is an optionally substituted 4-8 member monocyclic cycloalkyl group (e.g. ring A is cyclobutyl, cyclopentyl, cyclohexyl, cycloheptyl, cyclooctyl) or a 4-8 member monocyclic heterocyclyl group, wherein the heterocyclyl group contains 1 or 2 heteroatoms selected from O, S and N (e.g. ring A is oxetanyl, tetrahydrofuranyl, tetrahydropyranyl, 1,4-dioxanyl, pyrrolidinyl, piperidinyl, piperazinyl, morpholinyl, azepanyl). In some embodiments, ring A is tetrahydrofuranyl. In some embodiments, ring A is tetrahydropyranyl. In some embodiments, ring A is pyrrolidinyl. In some embodiments, ring A is cyclopentyl. In some embodiments, the monocyclic cycloalkyl or monocyclic heterocyclyl is not further substituted. In some embodiments, the cycloalkyl or heterocyclyl is further substituted with 0, 1, 2 or 3 substituents selected from halo (e.g., —F, —Cl), —OMe, —OEt —O(CH₂)OMe, —O(CH₂)₂OMe and CN.

In some embodiments, tetrahydrofuranyl is substituted with 1 or 2 substituents selected from halo (e.g., —F, —Cl), —OMe, -OEt —O(CH₂)OMe, —O(CH₂)₂OMe and CN. In some embodiments, tetrahydrofuranyl is substituted with 2 substituents selected from halo (e.g., —F, —Cl), —OMe, -OEt —O(CH₂)OMe, —O(CH₂)₂OMe and CN. In some embodiments, tetrahydrofuranyl is substituted with 1 substituent selected from halo (e.g., —F, —Cl), —OMe, -OEt —O(CH₂)OMe, —O(CH₂)₂OMe and CN. In some embodiments, tetrahydrofuranyl is substituted with —O(CH₂)₂OMe.

In some embodiments, the spacer is represented by Formula (I), wherein:

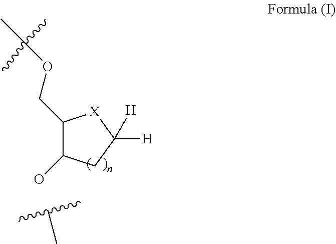

X is selected from —CH₂— and —O—; and
n is 0, 1, 2 or 3.

In some embodiments, the spacer is represented by Formula (I′), wherein:

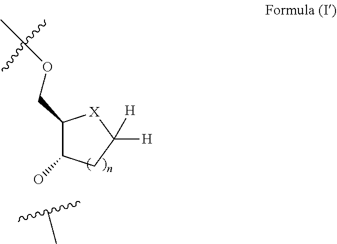

X is selected from —CH₂— and —O—; and
n is 0, 1, 2 or 3.

In some embodiments, the spacer is represented by Formula (Ia), wherein:

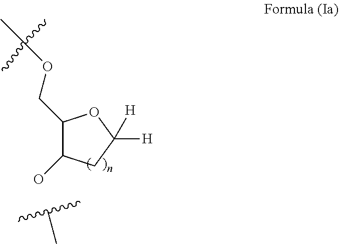

and n is 0, 1, 2 or 3.

In some embodiments, the spacer is represented by Formula (Ia′), wherein:

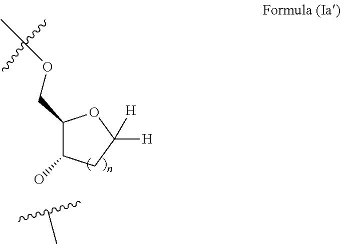

and n is 0, 1, 2 or 3.

As generally defined herein, X is selected from —CH₂— and —O—. In some embodiments, X is —CH₂—. In other embodiments, X is —O—.

As generally defined herein, n is 0, 1, 2 or 3. In some embodiments, n is 0. In some embodiments, n is 1 or 2. In some embodiments, n is 1. In other embodiments, n is 2. In certain embodiments, n is 3.

In some embodiments, the spacer is represented by Formula (II), wherein:

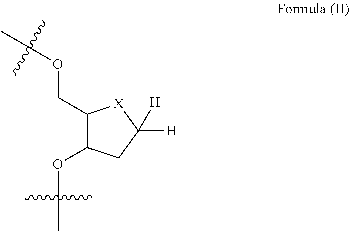

X is selected from —CH₂— and —O—.

In some embodiments, the spacer is represented by Formula (II′), wherein:

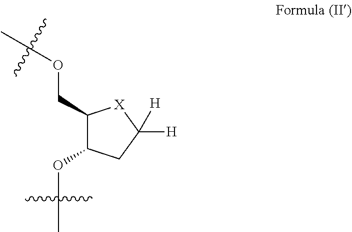

X is selected from —CH₂— and —O.

In some embodiments, the spacer is represented by Formula (Iia), wherein:

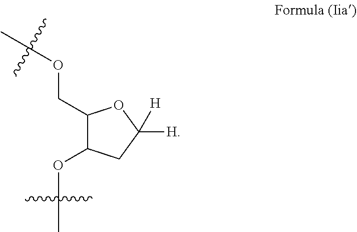

In some embodiments, the spacer is represented by Formula (Iia′), wherein:

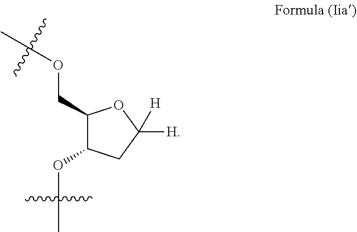

In some embodiments, the spacer is represented by Formula (IIi), wherein:

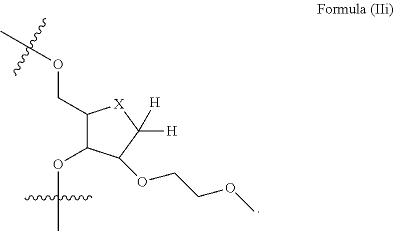

X is selected from —CH₂— and —O—.

In some embodiments, the spacer is represented by Formula (IIi′), wherein:

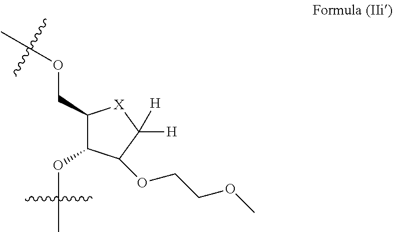

### Formula (IIi′)

X is selected from —CH₂— and —O.

In some embodiments, the spacer is represented by Formula (IIib), wherein:

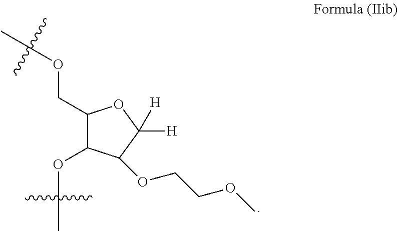 [SMILES: *OCC1OCC(OCCOC)C1O*]

In some embodiments, the spacer is represented by Formula (IIib′), wherein:

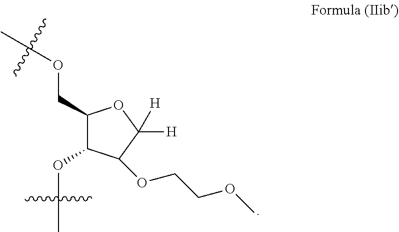 [SMILES: *OC[C@H]1OCC(OCCOC)[C@@H]1O*]

In some embodiments, the spacer is represented by Formula (III), wherein:

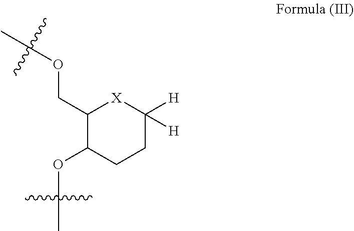

X is selected from —CH₂— and —O—.

In some embodiments, the spacer is represented by Formula (III′), wherein:

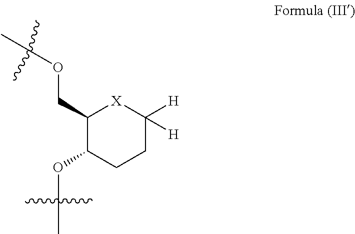

X is selected from —CH₂— and —O.

In some embodiments, the spacer is represented by Formula (IIIa), wherein:

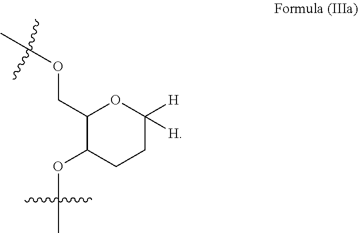

In some embodiments, the spacer is represented by Formula (IIIa′), wherein:

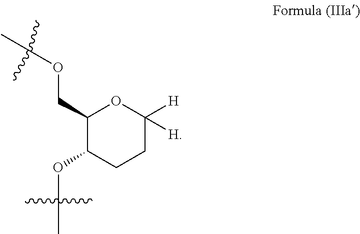

In some embodiments, the open positions of Formulae (I), (I′), (Ia), (Ia′), (II), (II′), (Iia), (Iia′), (III), (III′), (IIIa) and (IIIa′) (i.e., the positions not specifically depicted as bearing exclusively hydrogen atoms, including the —CH₂— group of X) are further substituted with 0-3 substituents independently selected from halo (e.g., —F, —Cl), -OMe, -OEt —O(CH₂)OMe, —O(CH₂)₂OMe and CN. In some embodiments, Formulae (I), (I′), (Ia), (Ia′), (II), (II′), (Iia), (Iia′), (III), (III′), (IIIa) and (IIIa′) are not further substituted.

As described further below, a UNC13A oligonucleotide with one or more spacers is described in reference to a corresponding UNC13A parent oligonucleotide or a corresponding UNC13A variant oligonucleotide. In various embodiments, a UNC13A oligonucleotide with a spacer differs from a UNC13A parent oligonucleotide or an UNC13A variant oligonucleotide in that the spacer replaces a nucleoside in the UNC13A parent oligonucleotide or an UNC13A variant oligonucleotide. As used hereafter, the “position” of the UNC13A oligonucleotide refers to a particular location as counted from the 5′ end of the UNC13A oligonucleotide. In various embodiments, the spacer replaces a nucleoside at any one of positions 1, 2, 3, 4, 5, 6, 7, 8, 9, 10, 11, 12, 13, 14, 15, 16, 17, 18, 19, 20, 21, 22, 23, 24, or 25 of the UNC13A parent oligonucleotide or an UNC13A variant oligonucleotide. In particular embodiments, a spacer replaces a nucleoside at one of positions 7, 8, 11, 14, 16, 19, or 22 of the UNC13A parent oligonucleotide or an UNC13A variant oligonucleotide.

In various embodiments, a UNC13A oligonucleotide includes one spacer that replaces a nucleoside in the UNC13A oligonucleotide (e.g., one spacer replaces one nucleoside of the UNC13A oligonucleotide). In particular embodiments, the spacer replaces a nucleoside between positions 9 and 15 of the UNC13A oligonucleotide. In particular embodiments, the spacer replaces a nucleoside between positions 9 and 12 of the UNC13A oligonucleotide. In particular embodiments, the spacer replaces a nucleoside at position 10 of the UNC13A oligonucleotide. In particular embodiments, the spacer replaces a nucleoside at position 11 of the UNC13A oligonucleotide. In particular embodiments, the spacer replaces a nucleoside at position 12 of the UNC13A oligonucleotide. In particular embodiments, the spacer replaces a nucleoside between positions 12 and 16 of the UNC13A oligonucleotide. In particular embodiments, the spacer replaces a nucleoside at position 15 of the UNC13A oligonucleotide.

In various embodiments, a UNC13A oligonucleotide including one spacer has 2 segments, where at least one of the 2 segments has at most 11 linked nucleosides. For example, the UNC13A oligonucleotide may be 23 oligonucleotide units in length, and the spacer can be located at position 12. Therefore, the UNC13A oligonucleotide has 2 segments divided by the spacer, where both of the 2 segments are 11 nucleobases in length. In various embodiments, a UNC13A oligonucleotide including one spacer has 2 segments, where at least one of the 2 segments has at most 10 linked nucleosides. For example, the UNC13A oligonucleotide may be 21 oligonucleotide units in length, and the spacer can be located at position 11. Therefore, the UNC13A oligonucleotide has 2 segments divided by the spacer, where both of the 2 segments are 10 nucleobases in length. As another example, the UNC13A oligonucleotide may be 25 oligonucleotide units in length, and the spacer can be located at position 15. Therefore, the UNC13A oligonucleotide has 2 segments divided by the spacer, where one of the 2 segments is 14 nucleobases in length and the second of the 2 segments is 10 nucleobases in length.

In various embodiments, a UNC13A oligonucleotide includes two spacers that each replace a nucleoside in the UNC13A oligonucleotide (e.g., two spacers replace two separate nucleosides of the UNC13A oligonucleotide). In various embodiments, a first spacer and a second spacer are separated by at least 5 nucleobases, at least 6 nucleobases, at least 7 nucleobases, at least 8 nucleobases, at least 9 nucleobases, or at least 10 nucleobases in the oligonucleotide. In particular embodiments, a first spacer and a second spacer are separated by at least 5 nucleobases, at least 6 nucleobases, or at least 7 nucleobases. In particular embodiments, the first spacer and the second spacer are not adjacent to one another in the oligonucleotide.

In particular embodiments, the first spacer replaces a nucleoside between positions 7 and 11 of the UNC13A oligonucleotide. In various embodiments, the first spacer replaces a nucleoside between positions 8 and 11, positions 9 and 11, positions 10 and 11, positions 7 and 10, positions 7 and 9, positions 7 and 8, positions 8 and 10, positions 8 and 9, or positions 9 and 10 of the UNC13A oligonucleotide. In particular embodiments, the second spacer replaces a nucleoside between positions 14 and 22 of the UNC13A oligonucleotide. In various embodiments, the second spacer replaces a nucleoside between positions 15 and 22, positions 16 and 22, positions 17 and 22, position 18 and 22, position 19 and 22, positions 20 and 22, positions 21 and 22, positions 15 and 21, position 16 and 21, positions 17 and 21, positions 18 and 21, positions 19 and 21, positions 20 and 21, positions 15 and 20, positions 16 and 20, positions 17 and 20, positions 18 and 20, positions 19 and 20, positions 15 and 19, positions 16 and 19, positions 17 and 19, positions 18 and 19, positions 15 and 18, position 16 and 18, position 17 and 18, positions 15 and 17, positions 16 and 17, or positions 15 and 16 of the UNC13A oligonucleotide.

In preferred embodiments, the first spacer replaces a nucleoside at position 7 of the UNC13A oligonucleotide and the second spacer replaces a nucleoside at position 14 of the UNC13A oligonucleotide. In preferred embodiments, the first spacer replaces a nucleoside at position 7 of the UNC13A oligonucleotide and the second spacer replaces a nucleoside at position 19 of the UNC13A oligonucleotide. In preferred embodiments, the first spacer replaces a nucleoside at position 8 of the UNC13A parent oligonucleotide and the second spacer replaces a nucleoside at position 16 of the UNC13A parent oligonucleotide. In preferred embodiments, the first spacer replaces a nucleoside at position 8 of the UNC13A oligonucleotide and the second spacer replaces a nucleoside at position 15 of the UNC13A oligonucleotide. In preferred embodiments, the first spacer replaces a nucleoside at position 11 of the UNC13A oligonucleotide and the second spacer replaces a nucleoside at position 22 of the UNC13A oligonucleotide. In preferred embodiments, the first spacer replaces a nucleoside at position 9 of the UNC13A oligonucleotide and the second spacer replaces a nucleoside at position 19 of the UNC13A oligonucleotide.

In various embodiments, a UNC13A oligonucleotide includes three spacers that each replace a nucleoside in the UNC13A oligonucleotide (e.g., three spacers replace three separate nucleosides of the UNC13A oligonucleotide). In particular embodiments, the first spacer replaces a nucleoside between positions 7 and 11 of the UNC13A oligonucleotide. In particular embodiments, the second spacer replaces a nucleoside between positions 14 and 22 of the UNC13A oligonucleotide. In particular embodiments, the third spacer replaces a nucleoside between positions 21 and 24 of the UNC13A oligonucleotide. In some embodiments, the first spacer replaces a nucleoside between positions 2 and 5 of the UNC13A oligonucleotide. In particular embodiments, the second spacer replaces a nucleoside between positions 8 and 12 of the UNC13A oligonucleotide. In particular embodiments, the third spacer replaces a nucleoside between positions 18 and 22 of the UNC13A oligonucleotide.

In various embodiments, the three spacers in a UNC13A oligonucleotide are positioned such that each of the four segments of the UNC13A oligonucleotide are at most 7 linked nucleosides in length. For example, a UNC13A oligonucleotide may have a first segment with 7 linked nucleosides connected to a first spacer, then a second segment with 7 linked nucleosides connected on one end to the first spacer and connected on another end to a second spacer, then a third segment with 6 linked nucleosides connected on one end to the second spacer and connected on another end to a third spacer, then a fourth segment with 6 linked nucleosides connected to the third spacer.

In various embodiments, the one or more spacers are positioned in the oligonucleotide to replace one or more adenosine or thymine nucleosides (as opposed to guanine or cytosine nucleosides). For example, the one or more spacers can replace one, two, three, four, five, six, seven, eight, or nine adenosine or thymine nucleosides in the oligonucleotide. In various embodiments, the one or more spacers are positioned in the oligonucleotide to replace one or more guanine or cytosine nucleosides (as opposed to adenosine or thymine nucleosides). For example, the one or more spacers can replace one, two, three, four, five, six, seven, eight, or nine guanine or cytosine nucleosides in the oligonucleotide. In various embodiments, the spacers are positioned in the oligonucleotide to replace an equal number of adenosine/thymine nucleosides and guanine/cytosine nucleosides. For example, a first spacer in the oligonucleotide may replace an adenosine/thymine nucleoside and a second spacer in the oligonucleotide may replace a guanine/cytosine nucleoside.

In various embodiments, the one or more spacers are positioned in the oligonucleotide to control the sequence content in the oligonucleotide. For example, the one or more spacers are positioned such that at least one of the spacers is located adjacent to a guanine group. In various embodiments, an oligonucleotide with spacers can include one spacer adjacent to a guanine group, two spacers adjacent to guanine groups, three spacers adjacent to guanine groups, four spacers adjacent to guanine groups, or five spacers adjacent to guanine groups. In one embodiment, if counting from the 5′ end of the oligonucleotide, a spacer immediately precedes a guanine group in the sequence. Thus, in various embodiments, an oligonucleotide with spacers can include one spacer that immediately precedes a guanine group, two spacers that each immediately precede a guanine group, three spacers that each immediately precede a guanine group, four spacers that each immediately precede a guanine group, or five spacers that each immediately precede a guanine group. In one embodiment, if counting from the 5′ end of the oligonucleotide, a guanine group is immediately succeeded by a spacer. Thus, in various embodiments, an oligonucleotide with spacers can include one spacer that immediately succeeds a guanine group, two spacers that each immediately succeed a guanine group, three spacers that each immediately succeed a guanine group, four spacers that each immediately succeed a guanine group, or five spacers that each immediately succeed a guanine group. In various embodiments, the spacers in the oligonucleotide can be positioned to maximize the number of spacers adjacent to guanine groups.

In various embodiments, the one or more spacers are positioned in the oligonucleotide to replace one or more adenosine or thymine nucleosides such that the one or more spacers are located adjacent guanine groups. For example, two spacers can replace adenosine or thymine nucleosides in the oligonucleotide, each of the two spacers being located adjacent to a guanine group.

In various embodiments, the UNC13A oligonucleotide with one or more spacers has a particular GC content. As used herein, GC content (or guanine-cytosine content) is the percentage of nitrogenous bases in the oligonucleotide that are either guanine (G) or cytosine (C). In various embodiments, the UNC13A oligonucleotide with one or more spacers has at least 10% GC content, at least 20% GC content, at least 25% GC content, at least 30% GC content, at least 35% GC content, at least 40% GC content, at least 45% GC content, at least 50% GC content, at least 55% GC content, at least 60% GC content, at least 65% GC content, at least 75% GC content, at least 80% GC content, at least 85% GC content, at least 90% GC content, or at least 95% GC content. In particular embodiments, the UNC13A oligonucleotide with one or more spacers has at least 30% GC content. In particular embodiments, the UNC13A oligonucleotide with one or more spacers has at least 40% GC content. In various embodiments, the one or more spacers are positioned in the UNC13A oligonucleotide to maximize GC content. For example, instead of selecting a guanine or cytosine for replacement by a spacer in the UNC13A oligonucleotide, a thymine or adenine can be selected for replacement by a spacer.

In various embodiments, a UNC13A oligonucleotide with spacers is designed such that 1) each segment of the UNC13A oligonucleotide has at most 7 linked nucleosides and 2) at least two, three, or four spacers are positioned adjacent to a guanine group. In some embodiments, a UNC13A oligonucleotide with spacers is designed such that 1) each segment of the UNC13A oligonucleotide has at most 7 linked nucleosides and 2) each of two spacers precede a guanine group.

In various embodiments, the inclusion of one or more spacers in the UNC13A oligonucleotide does not decrease the effectiveness of the UNC13A oligonucleotide with the spacers in restoring full length UNC13A protein or full length UNC13A mRNA in comparison to the effect of a corresponding UNC13A parent oligonucleotide. In various embodiments, the inclusion of one or more spacers in the UNC13A oligonucleotide increases the effectiveness of the UNC13A oligonucleotide with the spacers in restoring full length UNC13A protein or full length UNC13A mRNA in comparison to the effect of a corresponding UNC13A parent oligonucleotide. In various embodiments, the inclusion of one or more spacers in the UNC13A oligonucleotide does not decrease the effectiveness of the UNC13A oligonucleotide with the spacers in reducing quantity of UNC13A transcripts in comparison to the effect of a corresponding UNC13A parent oligonucleotide. In various embodiments, the inclusion of one or more spacers in the UNC13A oligonucleotide increases the effectiveness of the UNC13A oligonucleotide with the spacers in reducing quantity of UNC13A transcripts in comparison to the effect of a corresponding UNC13A parent oligonucleotide.

Tables 3A, 3B, 4, and 5 document example UNC13A oligonucleotides with one or more spacers and their relation to corresponding UNC13A parent oligonucleotides. Each UNC13A oligonucleotide is assigned a sequence name. As used hereafter, the nomenclature of the sequence name is expressed as “X_spA” (for a UNC13A AON with one spacer), “X_spA_spB” (for a UNC13A AON with two spacers), or “X_spA_spB_spC” (for a UNC13A AON with three spacers). Here, “X” refers to the length of the UNC13A AON, “A” refers to the position in the UNC13A AON where the first spacer is located, “B” refers to the position in the UNC13A AON where the second spacer is located, and if present, “C” refers to the position in the UNC13A AON where the third spacer is located.

In various embodiments, UNC13A oligonucleotides include one spacer. In various embodiments, the UNC13A oligonucleotides are oligonucleotide variants, such as any one of a 23mer, 21mer, or 19mer. In various embodiments, the inclusion of a spacer divides up the UNC13A oligonucleotide into two separate segments, where at least one of the segments is at most 11 linked nucleosides in length. In various embodiments, the inclusion of a spacer divides up the UNC13A oligonucleotide into two separate segments, where at least one of the segments is at most 10 linked nucleosides in length.

In various embodiments, the spacer is located between positions 10 and 15 of the oligonucleotide. In various embodiments, the spacer is located between positions 10 and 12 of the oligonucleotide. In particular embodiments, the spacer is located at position 10 of the oligonucleotide. In particular embodiments, the spacer is located at position 11 of the oligonucleotide. In particular embodiments, the spacer is located at position 12 of the oligonucleotide. In particular embodiments, the spacer is located at position 15 of the oligonucleotide. Example UNC13A AONs with one spacer are documented below in Table 3A.

TABLE 3A
Identification of UNC13A AONs with one spacer. Here, each UNC13A AON has 2
segments, where at least one of the segments has at most 11 linked nucleosides.

| | | | Sequence* (where X |
|---|---|---|---|
| | | Se- | indicates a nucleoside |
| | | quence | of the UNC13A parent |
| | | ID | oligonucleotide and S_{y} |
| | Relation to | Number | indicates presence of a |
| | UNC13A | (SEQ | Spacer where y |
| | oligonucleotide | ID | denotes the position) |
| Sequence name | variant | NO) | (5′ → 3′) |
| UNC13A parent | N/A | 5066 | XXXXXXXXXXXXXXXXXXXXXXXXX |
| oligonucleotide | | | (25 mer) |
| UNC13A | Nucleoside at | 5067 | XXXXXXXXXXXXXXXXXXXXXXXX |
| Oligonucleotide (25 mer) | position 15 of | | |
| with Spacer at position | 25 mer is | | |
| 15 | substituted with | | |
| (UNC13A AON | a spacer | | |
| 25_sp15) | | | |
| UNC13A | N/A | 5068 | XXXXXXXXXXXXXXXXXXXXXXX |
| oligonucleotide variant | | | (23 mer) |
| (23_mer) | | | |
| UNC13A | Nucleoside at | 5069 | XXXXXXXXXXXS₁₅XXXXXXXXXXX |
| Oligonucleotide (23 mer) | position 12 of | | |
| with Spacer at position | 23 mer is | | |
| 12 | substituted with | | |
| (UNC13A AON | a spacer | | |
| 23_sp12) | | | |
| UNC13A | N/A | 5070 | XXXXXXXXXXXXXXXXXXXXX |
| oligonucleotide variant | | | (21 mer) |
| (21_mer) | | | |
| UNC13A | Nucleoside at | 5071 | XXXXXXXXXXS₁₁XXXXXXXXXX |
| Oligonucleotide (21 mer) | position 11 of | | |
| with Spacer at position | 21 mer is | | |
| 11 | substituted with | | |
| (UNC13A AON | a spacer | | |
| 21_sp11) | | | |
| UNC13A oligonucleotide | N/A | 5072 | XXXXXXXXXXXXXXXXXXX |
| variant (19 mer) | | | (19 mer) |
| UNC13A | Nucleoside at | 5073 | XXXXXXXXXS₁₀XXXXXXXXX |
| Oligonucleotide (19 mer) | position 10 of | | |
| with Spacer at position | 19 mer is | | |
| 10 | substituted with | | |
| (UNC13A AON | a spacer | | |
| 19_sp10) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, UNC13A oligonucleotides include two spacers. In various embodiments, the inclusion of a spacer divides up the INC13A oligonucleotide into three separate segments, where at least one of the segments is at most 7 linked nucleosides in length. Example UNC13A AONs with two spacers are documented below in Table 3B.

TABLE 3B
Identification of UNC13A AONs with two spacers. Here, each UNC13A AON has 3
segments, where at least one of the segments has at most 7 linked nucleosides.

| | | | Sequence* (where X indicates a |
|---|---|---|---|
| | | | nucleoside of the UNC13A parent |
| | Relation to | Sequence ID | oligonucleotide and Sy indicates |
| | UNC13A parent | Number | presence of a Spacer where y denotes |
| Sequence name | oligonucleotide | (SEQ ID NO) | the position) (5′ → 3′) |
| UNC13A parent | N/A | 5074 | XXXXXXXXXXXXXXXXXXXXXXXXX |
| oligonucleotide | | | |
| UNC13A | Nucleosides at | 5075 | XXXXXXXXXXS11XXXXXXXXXXS22XXX |
| Oligonucleotide | positions 11 and | | |
| with Spacers at | 22 are each | | |
| positions 11 and 22 | substituted with | | |
| (UNC13A AON | a spacer | | |
| 25_sp11sp22) | | | |
| UNC13A | Nucleosides at | 5076 | XXXXXXS7XXXXXXS14XXXXXXXXXXX |
| Oligonucleotide | positions 7 and | | |
| with Spacers at | 14 are each | | |
| positions 7 and 14 | substituted with | | |
| (UNC13A AON | a spacer | | |
| 25_sp7sp14) | | | |
| UNC13A | Nucleosides at | 5077 | XXXXXXXS8XXXXXXXXXXS19XXXXXX |
| Oligonucleotide | positions 8 and | | |
| with Spacers at | 19 are | | |
| positions 8 and 19 | substituted with | | |
| (UNC13A AON | spacers | | |
| 25_sp8sp19) | | | |
| UNC13A | Nucleosides at | 5078 | XXXXXXXS9XXXXXS14XXXXXXXXXXX |
| Oligonucleotide | positions 8 and | | |
| with Spacers at | 14 are | | |
| positions 8 and 14 | substituted with | | |
| (UNC13A AON | spacers | | |
| 25_sp8sp14) | | | |
| UNC13A | Nucleosides at | 5079 | XXXXXXXXS9XXXXS14XXXXXXXXXXX |
| Oligonucleotide | positions 9 and | | |
| with Spacers at | 14 are | | |
| positions 9 and 14 | substituted with | | |
| (UNC13A AON | spacers | | |
| 25_sp9sp14) | | | |
| UNC13A | Nucleosides at | 5080 | XXXXXXXXXS10XXXS14XXXXXXXXXXX |
| Oligonucleotide | positions 10 and | | |
| with Spacers at | 14 are | | |
| positions 10 and 14 | substituted with | | |
| (UNC13A AON | spacers | | |
| 25_sp10sp14) | | | |
| UNC13A | Nucleosides at | 5081 | XXXXXXXXXXS11XXS14XXXXXXXXXXX |
| Oligonucleotide | positions 11 and | | |
| with Spacers at | 14 are | | |
| positions 11 and 14 | substituted with | | |
| (UNC13A AON | spacers | | |
| 25_sp11sp14) | | | |
| UNC13A | Nucleosides at | 5082 | XXXXXXXS8XXXXXXS15XXXXXXXXXX |
| Oligonucleotide | positions 8 and | | |
| with Spacers at | 15 are | | |
| positions 8 and 15 | substituted with | | |
| (UNC13A AON | spacers | | |
| 25_sp8sp15) | | | |
| UNC13A | Nucleosides at | 5083 | XXXXXXXXS9XXXXXS15XXXXXXXXXX |
| Oligonucleotide | positions 9 and | | |
| with Spacers at | 15 are | | |
| positions 9 and 15 | substituted with | | |
| (UNC13A AON | spacers | | |
| 25_sp9sp15) | | | |
| UNC13A | Nucleosides at | 5084 | XXXXXXXXXS10XXXXS15XXXXXXXXXX |
| Oligonucleotide | positions 10 and | | |
| with Spacers at | 15 are | | |
| positions 10 and 15 | substituted with | | |
| (UNC13A AON | spacers | | |
| 25_sp10sp15) | | | |
| UNC13A | Nucleosides at | 5085 | XXXXXXXXXXS11XXXS15XXXXXXXXXX |
| Oligonucleotide | positions 11 and | | |
| with Spacers at | 15 are | | |
| positions 11 and 15 | substituted with | | |
| (UNC13A AON | spacers | | |
| 25_sp11sp15) | | | |
| UNC13A | Nucleosides at | 5086 | XXXXXXXS8XXXXXXXS16XXXXXXXXX |
| Oligonucleotide | positions 8 and | | |
| with Spacers at | 16 are | | |
| positions 8 and 16 | substituted with | | |
| (UNC13A AON | spacers | | |
| 25_sp8sp16) | | | |
| UNC13A | Nucleosides at | 5087 | XXXXXXXXS9XXXXXXS16XXXXXXXXX |
| Oligonucleotide | positions 9 and | | |
| with Spacers at | 16 are | | |
| positions 9 and 16 | substituted with | | |
| (UNC13A AON | spacers | | |
| 25_sp9sp16) | | | |
| UNC13A | Nucleosides at | 5088 | XXXXXXXXXS10XXXXXS16XXXXXXXXX |
| Oligonucleotide | positions 10 and | | |
| with Spacers at | 16 are | | |
| positions 10 and 16 | | | |
| (UNC13A AON | substituted with | | |
| 25_sp10sp16) | spacers | | |
| UNC13A | Nucleosides at | 5089 | XXXXXXXXXXS11XXXXS16XXXXXXXXX |
| Oligonucleotide | positions 11 and | | |
| with Spacers at | 16 are | | |
| positions 11 and 16 | substituted with | | |
| (UNC13A AON | spacers | | |
| 25_spl1sp16) | | | |
| UNC13A | Nucleosides at | 5090 | XXXXXXXS8XXXXXXXXS17XXXXXXXX |
| Oligonucleotide | positions 8 and | | |
| with Spacers at | 17 are | | |
| positions 8 and 17 | substituted with | | |
| (UNC13A AON | spacers | | |
| 25_sp8sp17) | | | |
| UNC13A | Nucleosides at | 5091 | XXXXXXXXS9XXXXXXXS17XXXXXXXX |
| Oligonucleotide | positions 9 and | | |
| with Spacers at | 17 are | | |
| positions 9 and 17 | substituted with | | |
| (UNC13A AON | spacers | | |
| 25_sp9sp17) | | | |
| UNC13A | Nucleosides at | 5092 | XXXXXXXXXS10XXXXXXS17XXXXXXXX |
| Oligonucleotide | positions 10 and | | |
| with Spacers at | 17 are | | |
| positions 10 and 17 | substituted with | | |
| (UNC13A AON | spacers | | |
| 25_sp10sp17) | | | |
| UNC13A | Nucleosides at | 5093 | XXXXXXXXXXS11XXXXXS17XXXXXXXX |
| Oligonucleotide | positions 11 and | | |
| with Spacers at | 17 are | | |
| positions 11 and 17 | substituted with | | |
| (UNC13A AON | spacers | | |
| 25_sp11sp17) | | | |
| UNC13A | Nucleosides at | 5094 | XXXXXXXS8XXXXXXXXXXS18XXXXXXX |
| Oligonucleotide | positions 8 and | | |
| with Spacers at | 18 are | | |
| positions 8 and 18 | substituted with | | |
| (UNC13A ATON | spacers | | |
| 25_sp8sp18) | | | |
| UNC13A | Nucleosides at | 5095 | XXXXXXXXS9XXXXXXXXS18XXXXXXX |
| Oligonucleotide | positions 9 and | | |
| with Spacers at | 18 are | | |
| positions 9 and 18 | substituted with | | |
| (UNC13A AON | spacers | | |
| 25_sp9sp18) | | | |
| UNC13A | Nucleosides at | 5096 | XXXXXXXXXS10XXXXXXXS18XXXXXXX |
| Oligonucleotide | positions 10 and | | |
| with Spacers at | 18 are | | |
| positions 10 and 18 | substituted with | | |
| (UNC13A AON | spacers | | |
| 25_sp10sp18) | | | |
| UNC13A | Nucleosides at | 5097 | XXXXXXXXXXS11XXXXXXS18XXXXXXX |
| Oligonucleotide | positions 11 and | | |
| with Spacers at | 18 are | | |
| positions 11 and 18 | substituted with | | |
| (UNC13A AON | spacers | | |
| 25_spl1sp18) | | | |
| UNC13A | Nucleosides at | 5098 | XXXXXXXXS9XXXXXXXXXS19XXXXXX |
| Oligonucleotide | positions 9 and | | |
| with Spacers at | 19 are | | |
| positions 9 and 19 | substituted with | | |
| (UNC13A AON | spacers | | |
| 25_sp9sp19) | | | |
| UNC13A | Nucleosides at | 5099 | XXXXXXXXXS10XXXXXXXXS19XXXXXX |
| Oligonucleotide | positions 10 and | | |
| with Spacers at | 19 are | | |
| positions 10 and 19 | substituted with | | |
| (UNC13A AON | spacers | | |
| 25_sp10sp19) | | | |
| UNC13A | Nucleosides at | 5100 | XXXXXXXXXXS11XXXXXXXS19XXXXXX |
| Oligonucleotide | positions 11 and | | |
| with Spacers at | 19 are | | |
| positions 11 and 19 | substituted with | | |
| (UNC13A AON | spacers | | |
| 25_sp11sp19) | | | |
| UNC13A | Nucleosides at | 5101 | XXXXXXXXS9XXXXXXXXXXS20XXXXX |
| Oligonucleotide | positions 9 and | | |
| with Spacers at | 20 are | | |
| positions 9 and 20 | substituted with | | |
| (UNC13A AON | spacers | | |
| 25_sp9sp20) | | | |
| UNC13A | Nucleosides at | 5102 | XXXXXXXXXS10XXXXXXXXXS20XXXXX |
| Oligonucleotide | positions 10 and | | |
| with Spacers at | 20 are | | |
| positions 10 and 20 | substituted with | | |
| (UNC13A AON | spacers | | |
| 25_sp10sp20) | | | |
| UNC13A | Nucleosides at | 5103 | XXXXXXXXXXS11XXXXXXXXS20XXXXX |
| Oligonucleotide | positions 11 and | | |
| with Spacers at | 20 are | | |
| positions 11 and 20 | substituted with | | |
| (UNC13A AON | spacers | | |
| 25_sp11sp20) | | | |
| UNC13A | Nucleosides at | 5104 | XXXXXXXXXS10XXXXXXXXXXS21XXXX |
| Oligonucleotide | positions 10 and | | |
| with Spacers at | 21 are | | |
| positions 10 and 21 | substituted with | | |
| (UNC13A AON | spacers | | |
| 25_sp10sp21) | | | |
| UNC13A | Nucleosides at | 5105 | XXXXXXXXXXS11XXXXXXXXXS21XXXX |
| Oligonucleotide | positions 11 and | | |
| with Spacers at | 21 are | | |
| positions 11 and 21 | substituted with | | |
| (UNC13A AON | spacers | | |
| 25_spl1sp21) | | | |
| UNC13A | Nucleosides at | 5106 | XXXS₄XXXXXXXXXXS₁₅XXXXXXXXXX |
| Oligonucleotide | positions 4 and | | |
| with Spacers at | 15 are | | |
| positions 4 and 15 | substituted with | | |
| (UNC13A AON | spacers | | |
| 25_sp4sp15) | | | |
| UNC13A | Nucleosides at | 5107 | XXXXXXS7XXXXXXXXXXXS19XXXXXX |
| Oligonucleotide | positions 7 and | | |
| with Spacers at | 19 are | | |
| positions 7 and 19 | substituted with | | |
| (UNC13A AON | spacers | | |
| 25_sp7sp19) | | | |
| UNC13A | Nucleosides at | 5108 | XXXXXXS7XXXXXXXXXXS18XXXXXXX |
| Oligonucleotide | positions 7 and | | |
| with Spacers at | 18 are | | |
| positions 7 and 18 | substituted with | | |
| (UNC13A AON | spacers | | |
| 25_sp7sp18) | | | |
| UNC13A | Nucleosides at | 5109 | XXXXXXXXS9XXXXXXXXXXXS21XXXX |
| Oligonucleotide | positions 9 and | | |
| with Spacers at | 21 are | | |
| positions 9 and 21 | substituted with | | |
| (UNC13A AON | spacers | | |
| 25_sp9sp21) | | | |

*At least one nucleoside linkage of the nucleobase sequence is selected from a phosphorothioate
linkage, an alkyl phosphate linkage, a phosphorodithioate linkage, a phosphotriester linkage, an
alkylphosphonate linkage, a 3-methoxypropyl phosphonate linkage, a methylphosphonate linkage, an
aminoalkylphosphotriester linkage, an alkylene phosphonate linkage, a phosphinate linkage, a
phosphoramidate linkage, a phosphoramidothioate linkage, a phosphorodiamidate (e.g., comprising
a phosphorodiamidate morpholino (PMO), 3′ amino ribose, or 5′ amino ribose) linkage, an
aminoalkylphosphoramidate linkage, a thiophosphoramidate linkage, a thionoalkylphosphonate
linkage, a thionoalkylphosphotriester linkage, a thiophosphate linkage, a selenophosphate
linkage, and a boranophosphate linkage.

In various embodiments, UNC13A oligonucleotides include three spacers. The inclusion of three spacers divides up the UNC13A oligonucleotide into four separate segments. In various embodiments, the three spacers are located at different positions of the UNC13A oligonucleotide such that each of the segments of the UNC13A oligonucleotide are at most 7 linked nucleosides in length. Example UNC13A AONs with three spacers are documented below in Table 4.

TABLE 4
Identification of UNC13A AONs or AON variants with three spacers. Here, each
UNC13A AON has 4 segments, where each segment has at most 7 linked nucleosides.

| | | | Sequence* (where X indicates a |
|---|---|---|---|
| | Relation to | | nucleoside of the UNC13A parent |
| | UNC13A | Sequence ID | oligonucleotide and S_{y} indicates |
| | parent | Number | presence of a Spacer where y denotes |
| Sequence name | oligonucleotide | (SEQ ID NO) | the position) (5′ → 3′) |
| UNC13A | Nucleosides | 5110 | XXXXXXXS₈XXXXXXXS₁₆XXXXXXXS₂₄X |
| Oligonucleotide | at positions 8 | | |
| with Spacers at | and 16 and 24 | | |
| positions 8 and 16 | are substituted | | |
| and 24 (UNC13A | with spacers | | |
| AON | | | |
| 25_sp8sp16sp24) | | | |
| UNC13A | Nucleosides | 5111 | XXXXXXXS₈XXXXXXXS₁₆XXXXXXS₂₃XX |
| Oligonucleotide | at positions 8 | | |
| with Spacers at | and 16 and 23 | | |
| positions 8 and 16 | are substituted | | |
| and 23 (UNC13A | with spacers | | |
| AON | | | |
| 25_sp8sp16sp23) | | | |
| UNC13A | Nucleosides | 5112 | XS₂XXXXXXXS₁₀XXXXXXXS₁₈XXXXXXX |
| Oligonucleotide | at positions 2 | | |
| with Spacers at | and 10 and 18 | | |
| positions 2 and 10 | are substituted | | |
| and 18 (UNC13A | with spacers | | |
| AON | | | |
| 25_sp8sp16sp23) | | | |
| UNC13A | Nucleosides | 5113 | XXS₃XXXXXXS₁₀XXXXXXXS₁₈XXXXXXX |
| Oligonucleotide | at positions 3 | | |
| with Spacers at | and 10 and 18 | | |
| positions 3 and 10 | are substituted | | |
| and 18 (UNC13A | with spacers | | |
| AON | | | |
| 25_sp8sp16sp23) | | | |
| UNC13A | Nucleosides | 5114 | XXXS₄XXXXXXXS₁₂XXXXXXS₁₉XXXXXX |
| Oligonucleotide | at positions 4 | | |
| with Spacers at | and 12 and 19 | | |
| positions 4 and 12 | are substituted | | |
| and 19 (UNC13A | with spacers | | |
| AON | | | |
| 25_sp4sp12sp19) | | | |
| UNC13A | Nucleosides | 5115 | XXXXXXXS₈XXXXS₁₃XXXXS₁₈XXXXXXX |
| Oligonucleotide | at positions 8 | | |
| with Spacers at | and 13 and 18 | | |
| positions 8 and 13 | are substituted | | |
| and 18 (UNC13A | with spacers | | |
| AON | | | |
| 25_sp8sp13sp18) | | | |
| UNC13A | Nucleosides | 5116 | XXXXS₅XXXXXXXS₁₃XXXXXXXS₂₁XXXX |
| Oligonucleotide | at positions 5 | | |
| with Spacers at | and 13 and 21 | | |
| positions 5 and 13 | are substituted | | |
| and 21 (UNC13A | with spacers | | |
| AON | | | |
| 25_sp5sp13sp21) | | | |
| UNC13A | Nucleosides | 5117 | XXXXXXS₇XXXXXS₁₃XXXXXS₁₉XXXXXX |
| Oligonucleotide | at positions 7 | | |
| with Spacers at | and 13 and 19 | | |
| positions 7 and 13 | are substituted | | |
| and 19 (UNC13A | with spacers | | |
| AON | | | |
| 25_sp7sp13sp19) | | | |
| UNC13A | Nucleosides | 5118 | XXXXXS₆XXXXXXS₁₃XXXXXXS₂₀XXXXX |
| Oligonucleotide | at positions 6 | | |
| with Spacers at | and 13 and 20 | | |
| positions 6 and 13 | are substituted | | |
| and 20 (UNC13A | with spacers | | |
| AON | | | |
| 25_sp6sp13sp20) | | | |
| UNC13A | Nucleosides | 5119 | XXXXXXXS₈XXS₁₁XXXXXXXS₁₉XXXXXX |
| Oligonucleotide | at positions 8 | | |
| with Spacers at | and 11 and 19 | | |
| positions 8 and 11 | are substituted | | |
| and 19 (UNC13A | with spacers | | |
| AON | | | |
| 25_sp8sp11sp19) | | | |
| UNC13A | Nucleosides | 5120 | XXXXXXXS₈XXS₁₁XXXXS₁₆XXXXXXX |
| Oligonucleotide | at positions 8 | | |
| with Spacers at | and 11 and 16 | | |
| positions 8 and 11 | are substituted | | |
| and 16 (UNC13A | with spacers | | |
| AON | | | |
| 23_sp8sp11sp16) | | | |
| UNC13A | Nucleosides | 5121 | XXXXXXS₇XXXXXXS₁₄XXXXXXXS₂₂XXX |
| Oligonucleotide | at positions 7 | | |
| with Spacers at | and 14 and 22 | | |
| positions 7 and 14 | are substituted | | |
| and 22 (UNC13A | with spacers | | |
| AON | | | |
| 23_sp7sp14sp22) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In various embodiments, UNC13A AONs with one or more spacers are reduced in length in comparison to the UNC13A AONs described above in Tables 3B and 4. For example, such UNC13A AONs may be UNC13A oligonucleotide variants with one or more spacers. In various embodiments, the UNC13A oligonucleotide variants with one or more spacers are 23mers, 21mers, or 19mers. In various embodiments, UNC13A oligonucleotide variants include two spacers such that the UNC13A oligonucleotide variant includes three segments that are divided up by the two spacers. In various embodiments, at least one of the three segments has at most 7 linked nucleosides. In various embodiments, each of the three segments has at most 7 linked nucleosides. Example UNC13A oligonucleotide variants with one or more spacers are shown below in Table 5.

TABLE 5
Example UNC13A AON variants with two spacers. Here, each UNC13A AON variant
has 3 segments, where at least one segment has at most 7 linked nucleosides.

| | | | Sequence* (where X indicates a |
|---|---|---|---|
| | Relation to | | nucleoside of the UNC13A |
| | UNC13A | Sequence ID | oligonucleotide variant and Sy indicates |
| Sequence | oligonucleotide | Number | presence of a Spacer where y denotes the |
| name | variant | (SEQ ID NO) | position) (5′ → 3′) |
| UNC13A | N/A | 5122 | XXXXXXXXXXXXXXXXXXXXXXX (23 mer) |
| oligonucleotide | | | |
| variant (23 mer) | | | |
| UNC13A | Nucleosides at | 5123 | XXXXXXXS₈XXXXXXXS₁₆XXXXXXX |
| Variant | positions 8 and | | |
| Oligonucleotide | 16 are | | |
| (23 mer) with | substituted with | | |
| Spacers at | spacers | | |
| positions 8 and | | | |
| 16 (UNC13A | | | |
| AON variant | | | |
| 23_sp8sp16) | | | |
| UNC13A | N/A | 5124 | XXXXXXXXXXXXXXXXXXXXX |
| oligonucleotide | | | (21 mer) |
| variant (21 mer) | | | |
| UNC13A | Nucleosides at | 5125 | XXXXS₅XXXXXXS₁₂XXXXXXXXX |
| Variant | positions 5 and | | |
| Oligonucleotide | 12 are | | |
| (21 mer) with | substituted with | | |
| Spacers at | spacers | | |
| positions 5 and | | | |
| 12 (UNC13A | | | |
| AON variant | | | |
| 21_sp5sp12) | | | |
| UNC13A | Nucleosides at | 5126 | XXXXXXXS₈XXXXXXXS₁₆XXXXX |
| Variant | positions 8 and | | |
| Oligonucleotide | 16 are | | |
| (21 mer) with | substituted with | | |
| Spacers at | spacers | | |
| positions 8 and | | | |
| 16 (UNC13A | | | |
| AON variant | | | |
| 21_sp8sp16) | | | |
| UNC13A | Nucleosides at | 5127 | XXXXXS₆XXXXXXXS₁₄XXXXXXX |
| Variant | positions 6 and | | |
| Oligonucleotide | 14 are | | |
| (21 mer) with | substituted with | | |
| Spacers at | spacers | | |
| positions 6 and | | | |
| 14 (UNC13A | | | |
| AON variant | | | |
| 21_sp6sp14) | | | |
| UNC13A | Nucleosides at | 5128 | XXXXXXXS₈XXXXXS₁₄XXXXXXX |
| Variant | positions 8 and | | |
| Oligonucleotide | 14 are | | |
| (21 mer) with | substituted with | | |
| Spacers at | spacers | | |
| positions 8 and | | | |
| 14 (UNC13A | | | |
| AON variant | | | |
| 21_sp8sp14) | | | |
| UNC13A | Nucleosides at | 5129 | XXXXXS₆XXXXXXXXXXXXXS₂₀X |
| Variant | positions 6 and | | |
| Oligonucleotide | 20 are | | |
| (21 mer) with | substituted with | | |
| Spacers at | spacers | | |
| positions 8 and | | | |
| 14 (UNC13A | | | |
| AON variant | | | |
| 21_sp8sp14) | | | |
| UNC13A | N/A | 5130 | XXXXXXXXXXXXXXXXXXX |
| oligonucleotide | | | (19 mer) |
| variant (19 mer) | | | |
| UNC13A | Nucleosides at | 5131 | XXXXS₅XXXXXXS₁₂XXXXXXX |
| Variant | positions 5 and | | |
| Oligonucleotide | 12 are | | |
| (19 mer) with | substituted with | | |
| Spacers at | spacers | | |
| positions 5 and | | | |
| 12 (UNC13A | | | |
| AON variant | | | |
| 19_sp5sp12) | | | |
| UNC13A | Nucleosides at | 5132 | XXXXXXXS₈XXXXXXS₁₅XXXX |
| Variant | positions 8 and | | |
| Oligonucleotide | 15 are | | |
| (19 mer) with | substituted with | | |
| Spacers at | spacers | | |
| positions 8 and | | | |
| 15 (UNC13A | | | |
| AON variant | | | |
| 19_sp8sp15) | | | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In some embodiments, an antisense oligonucleotide disclosed herein (e.g., UNC13A parent oligonucleotides and/or UNC13A oligonucleotide variants) comprise one or more spacers as well as one or more locked nucleic acids (LNAs). In some embodiments, an antisense oligonucleotide disclosed herein (e.g., UNC13A parent oligonucleotides and/or UNC13A oligonucleotide variants) comprises two spacers and two LNAs. In some embodiments, an antisense oligonucleotide disclosed herein (e.g., UNC13A parent oligonucleotides and/or UNC13A oligonucleotide variants) comprises two spacers and three LNAs.

In various embodiments, a spacer and a LNA are located adjacent to one another in an antisense oligonucleotide. For example, if counting from 5′ to 3′, a LNA can be located at a 7^{th} position of the antisense oligonucleotide and a spacer can be located at a 8^{th} position of the antisense oligonucleotide. As another example, if counting from 5′ to 3′, a LNA can be located at a 9^{th} position of the antisense oligonucleotide and a spacer can be located at a 8^{th} position of the antisense oligonucleotide. As another example, if counting from 5′ to 3′, a LNA can be located at a 15^{th} position of the antisense oligonucleotide and a spacer can be located at a 16^{th} position of the antisense oligonucleotide. As another example, if counting from 5′ to 3′, a LNA can be located at a 17^{th} position of the antisense oligonucleotide and a spacer can be located at a 16^{th} position of the antisense oligonucleotide.

In particular embodiments, a first spacer is located adjacent to a first LNA and a second spacer is located adjacent to a second LNA in an antisense oligonucleotide. For example, if counting from 5′ to 3′, a first LNA can be located at a 7^{th} position of the antisense oligonucleotide, a first spacer can be located at a 8^{th} position of the antisense oligonucleotide, a second LNA can be located at a 15^{th} position of the antisense oligonucleotide, and a second spacer can be located at a 16^{th} position of the antisense oligonucleotide. As another example, if counting from 5′ to 3′, a first LNA can be located at a 7^{th} position of the antisense oligonucleotide, a first spacer can be located at a 8^{th} position of the antisense oligonucleotide, a second LNA can be located at a 17^{th} position of the antisense oligonucleotide, and a second spacer can be located at a 16^{th} position of the antisense oligonucleotide. As another example, if counting from 5′ to 3′, a first LNA can be located at a 9^{th} position of the antisense oligonucleotide, a first spacer can be located at a 8^{th} position of the antisense oligonucleotide, a second LNA can be located at a 17^{th} position of the antisense oligonucleotide, and a second spacer can be located at a 16^{th} position of the antisense oligonucleotide.

In various embodiments, one or more spacers and one or more LNAs are not located adjacent to one another in an antisense oligonucleotide. For example, if counting from 5′ to 3′, a LNA can be located at a 4^{th} position of the antisense oligonucleotide and a spacer can be located at a 8^{th} position of the antisense oligonucleotide. As example, if counting from 5′ to 3′, a LNA can be located at a 20^{th} position of the antisense oligonucleotide and a spacer can be located at a 16^{th} position of the antisense oligonucleotide. In particular embodiments, if counting from 5′ to 3′, a first LNA can be located at a 4^{th} position of the antisense oligonucleotide, a first spacer can be located at a 8^{th} position of the antisense oligonucleotide, a second LNA can be located at a 20^{th} position of the antisense oligonucleotide, and a second spacer can be located at a 16^{th} position of the antisense oligonucleotide. In particular embodiments, if counting from 5′ to 3′, a first LNA can be located at a 4^{th} position of the antisense oligonucleotide, a first spacer can be located at a 8^{th} position of the antisense oligonucleotide, a second LNA can be located at a 12^{th} position of the antisense oligonucleotide, a second spacer can be located at a 16^{th} position of the antisense oligonucleotide, and a third LNA can be located at a 20^{th} position of the antisense oligonucleotide.

### Performance of UNC13A Oligonucleotides

Generally, UNC13A oligonucleotides and/or UNC13A parent oligonucleotides (e.g., UNC13A oligonucleotides with sequences of any of SEQ ID NOs: 1-1264, SEQ ID NO: 2529-3792, SEQ ID NOs: 5066-5166, SEQ ID NOs: 5168-5202, SEQ ID NOs: 5209-5221, or SEQ ID NOs: 5235-5292) target UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to SEQ ID NO: 5057-5065 or SEQ ID NOs: 5206-5208 in order to increase, restore, rescue, or stabilize levels of expression of UNC13A mRNA that is capable of translation to produce a functional UNC13A protein (e.g., full length UNC13A). In various embodiments, UNC13A AONs can exhibit at least a 20%, 30%, 40%, 50%, 60%, 70%, 80%, or 90% increase of full length UNC13A mRNA. In various embodiments, UNC13A AONs can exhibit at least a 100%, 200%, 300%, or 400% increase of full length UNC13A mRNA. In various embodiments, UNC13A AONs can exhibit at least a 20%, 30%, 40%, 50%, 60%, 70%, 80%, or 90% reduction of mis-spliced UNC13A mRNA. In various embodiments, UNC13A AONs can exhibit at least a 20%, 30%, 40%, 50%, 60%, 70%, 80%, or 90% increase of full length UNC13A protein. In various embodiments, UNC13A AONs can exhibit at least a 100%, 200%, 300%, or 400% increase of full length UNC13A protein. In some embodiments, the percent increase of the full length UNC13A protein is an increase in comparison to a reduced level of full length UNC13A protein achieved using a TDP43 antisense oligonucleotide. For example, a TDP43 antisense oligonucleotide can be used to deplete full length UNC13A protein followed by increase of the full length UNC13A protein using a UNC13A AON.

In some embodiments, UNC13A AONs can exhibit at least a 10%, 20%, 30%, 40%, 50%, 60%, 70%, 80%, 90%, or 100% rescue of full length UNC13A protein. In some embodiments, the percent rescue of full length UNC13A refers to the % of full length UNC13A following depletion using a TDP43 antisense oligonucleotide and a treatment using UNC13A AONs in comparison to a negative control (e.g., cells that did not undergo depletion or treatment or cells that were treated with a vehicle solution).

In various embodiments, UNC13A AONs can exhibit at least a 30%, 40%, 50%, 60%, 70%, 80%, or 90% reduction of an UNC13A transcript with a cryptic exon. In various embodiments, UNC13A AONs can exhibit at least a 100% reduction of an UNC13A transcript with a cryptic exon. In various embodiments, reduction of an UNC13A transcript with a cryptic exon is measured in comparison to a level of UNC13A transcript with a cryptic exon detected using a TDP43 antisense oligonucleotide. In various embodiments, UNC13A AONs can exhibit at least a 10%, 20%, 30%, 40%, 50%, 60%, 70%, 80%, 90%, or 100% reduction of an UNC13A transcript with a cryptic exon.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′,3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a UNC13A nucleic acid comprise one or more modified internucleoside linkages. In certain embodiments, the modified internucleoside linkages are interspersed throughout the antisense compound. In certain embodiments, the modified internucleoside linkages are phosphorothioate linkages. In certain embodiments, each internucleoside linkage of an antisense compound is a phosphorothioate internucleoside linkage. In certain embodiments, the antisense compounds targeted to a UNC13A nucleic acid comprise at least one phosphodiester linkage and at least one phosphorothioate linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substituent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or 5), 4′-S, 2′-F, 2′-OCH₃, 2′-OCH₂CH₃, 2′—O CH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂S CH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ)—, where each Rₗ, Rₘ and Rₙ is, independently, H or substituted or unsubstituted C₁-C₁₀ alkyl.

Additional examples of modified sugar moieties include a 2′-OMe modified sugar moiety, bicyclic sugar moiety, 2′-O-(2-methoxyethyl) (2′-MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′—(CH₂)—O-2′ (LNA); 4′—(CH₂)—S-2′; 4′—(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′—CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′—CH₂—N(R)—O-2′, wherein R is H, C₁-C₁₂ alkyl, or a protecting group (see U.S. Pat. No. 7,427,672, issued on Sep. 23, 2008); 4′—CH₂—C(H)(CH₃)-2′ (see Chattopadhyaya et al., J. Org. Chem., 2009, 74, 118-134); and 4′—CH₂—C—(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. patent Ser. No. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—; wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[—[C(Rₐ)(R_{b})]ₙ—O—, —C(RₐR_{b})—N(R)—O— or —C(RₐR_{b})—O—N(R)—. In certain embodiments, the bridge is 4′—CH₂-2′,4′—(CH₂)₂-2′,4′—(CH₂)₃-2′,4′—CH₂—O-2′,4′—(CH₂)₂—O-2′,4′—CH₂—O—N(R)-2′ and 4′—CH₂—N(R)—O-2′— wherein each R is, independently, H, a protecting group or C₁-C₁₂ alkyl, each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group; and R is H, C₁-C₁₂ alkyl, or a protecting group (see U.S. Pat. No. 7,427,672, issued on Sep. 23, 2008).

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′—CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, α-L-methyleneoxy (4′—CH₂—O-2′) BNA, β-D-methyleneoxy (4′—CH₂—O-2′) BNA, ethyleneoxy (4′—(CH₂)₂—O-2) BNA, aminooxy (4′—CH₂—O—N(R)-2′) BNA, oxyamino (4′—CH₂—N(R)—O-2′) BNA, methyl(methyleneoxy) (4′-CH(CH₃)—O-2′) BNA, methylene-thio (4′—CH₂—S-2′) BNA, methylene-amino (4′—CH₂—N(R)-2′) BNA, methyl carbocyclic (4′—CH₂—CH(CH₃)-2′) BNA, and propylene carbocyclic (4′—(CH₂)₃-2′) BNA; wherein R is H, C₁-C₁₂ alkyl, or a protecting group (see U.S. Pat. No. 7,427,672, issued on Sep. 23, 2008).

The present disclosure provide, in some embodiments, methods for treating, ameliorating, or preventing a neurological disease and/or a neuropathy further include methods of administering, to a patient, a pharmaceutically acceptable composition, for example, a pharmaceutically acceptable formulation that includes one or more UNC13A oligonucleotides. UNC13A oligonucleotides can increase, restore, or stabilize UNC13A activity, for example, UNC13A activity, and/or levels of UNC13A expression, for example, UNC13A mRNA and/or protein expression.

The present disclosure also provides pharmaceutical compositions comprising a UNC13A oligonucleotide formulated together with one or more pharmaceutically or cosmetically acceptable excipients. These formulations include those suitable for oral, sublingual, intratracheal, intranasal, transdermal, pulmonary, intrathecal, intrathalamic, intracisternal, intracerebroventricular, parenteral (e.g., subcutaneous, intramuscular, intradermal, intraduodenal, or intravenous) administration, transmucosal (e.g., buccal, vaginal, and rectal), or for topical use, e.g., as part of a composition suitable for applying topically to skin and/or mucous membrane, for example, a composition in the form of a gel, a paste, a wax, a cream, a spray, a liquid, a foam, a lotion, an ointment, a topical solution, a transdermal patch, a powder, a vapor, or a tincture. Although the most suitable form of administration in any given case will depend on the degree and severity of the condition being treated and on the nature of the particular UNC13A oligonucleotide being used.

The present disclosure also provides a pharmaceutical composition comprising a UNC13A oligonucleotide or a pharmaceutically acceptable salt thereof (for example, a UNC13A AON that includes a sequence of any of SEQ ID NOs: 1-1264, SEQ ID NO: 2529-3792, SEQ ID NOs: 5066-5166, SEQ ID NOs: 5168-5202, SEQ ID NOs: 5209-5221, or SEQ ID NOs: 5235-5292).

The present disclosure also provides methods that include the use of pharmaceutical compositions comprising a UNC13A AON is formulated together with one or more pharmaceutically acceptable excipients. Exemplary compositions provided herein include compositions comprising a UNC13A AON, and one or more pharmaceutically acceptable excipients. Formulations include those suitable for oral, sublingual, intratracheal, intranasal, transdermal, pulmonary, intrathecal, intrathalamic, intracisternal, intracerebroventricular, parenteral (e.g., subcutaneous, intramuscular, intradermal, intraduodenal, or intravenous) administration, transmucosal (e.g., buccal, vaginal, and rectal), or for topical use. The most suitable form of administration in any given case will depend on the clinical symptoms, complications, or biochemical indicia of the state, disorder, disease, or condition that one is trying to prevent in a subject; the state, disorder, disease, or condition one is trying to prevent in a subject; and/or on the nature of the particular compound and/or the composition being used.

### Additional Chemically Modified UNC13A Oligonucleotides

UNC13A AONs described herein, can include chemically modified nucleosides, including modified ribonucleosides and modified deoxyribonucleosides. Chemically modified nucleosides include, but are not limited to, uracine, uridine, 2′-O-(2-methoxyethyl) modifications, for example, 2′-O-(2-methoxyethyl)guanosine, 2′-O-(2-methoxyethyl)adenosine, 2′-O-(2-methoxyethyl)cytosine, and 2′-O-(2-methoxyethyl)thymidine. In certain embodiments, mixed modalities, e.g., a combination of a UNC13A peptide nucleic acid (PNA) and a UNC13A locked nucleic acid (LNA). Chemically modified nucleosides also include, but are not limited to, locked nucleic acids (LNAs), 2′-O-methyl, 2′-fluoro, and 2′-fluoro-β-D-arabinonucleotide (FANA), and Fluoro Cyclohexenyl nucleic acid (F-CeNA) modifications. Chemically modified nucleosides that can be included in UNC13A AONs described herein are described in Johannes and Lucchino, (2018) “Current Challenges in Delivery and Cytosolic Translocation of Therapeutic RNAs” Nucleic Acid Ther. 28(3): 178-93; Rettig and Behlke, (2012) “Progress toward in vivo use of siRNAs-II” Mol Ther 20:483-512; and Khvorova and Watts, (2017) “The chemical evolution of oligonucleotide therapies of clinical utility” Nat Biotechnol., 35(3):238-48, the contents of each of which are incorporated by reference herein.

UNC13A AONs described herein can include chemical modifications that promote stabilization of an oligonucleotide's terminal 5′-phosphate and phosphatase-resistant analogs of 5′-phosphate. Chemical modifications that promote oligonucleotide terminal 5′-phosphate stabilization or which are phosphatase-resistant analogs of 5′-phosphate include, but are not limited to, 5′-methyl phosphonate, 5′-methylenephosphonate, 5′-methylenephosphonate analogs, 5′-E-vinyl phosphonate (5′-E-VP), 5′-phosphorothioate, and 5′-C-methyl analogs. Chemical modifications that promote AON terminal 5′-phosphate stabilization and phosphatase-resistant analogues of 5′-phosphate are described in Khvorova and Watts, (2017) “The chemical evolution of oligonucleotide therapies of clinical utility” Nat Biotechnol., 35(3):238-48, the contents of which are incorporated by reference herein.

In some embodiments described herein, UNC13A AONs described herein can include chemically modified nucleosides, for example, 2′ O-methyl ribonucleosides, for example, 2′ O-methyl cytidine, 2′ O-methyl guanosine, 2′ O-methyl uridine, and/or 2′ O-methyl adenosine. UNC13A AONs described herein can include one or more chemically modified bases, including a 5-methylpyrimidine, for example, 5-methylcytosine, and/or a 5-methylpurine, for example, 5-methylguanine. Chemically modified nucleosides can further include pseudo-uridine or 5′ methoxyuridine. UNC13A AONs described herein can include any of the following chemically modified nucleosides: 5-methyl-2′-O-methylcytidine, 5-methyl-2′-O-methylthymidine, 5-methylcytidine, 5-methyluridine, and/or 5-methyl 2′-deoxycytidine.

UNC13A AONs described herein can include a phosphate backbone where one or more of the oligonucleoside linkages is a phosphate linkage. UNC13A AONs described herein may include a modified oligonucleotide backbone, where one or more of the nucleoside linkages of the sequence is selected from the group consisting of a phosphorothioate linkage, a phosphorodithioate linkage, a phosphotriester linkage, an alkylphosphonate linkage, a 3-methoxypropyl phosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments of UNC13A AONs described herein, at least one (i.e., one or more) internucleoside linkage of the oligonucleotide is a phosphorothioate linkage. For example, in some embodiments of UNC13A AONs described herein, one, two, three, or more internucleoside linkages of the oligonucleotide is a phosphorothioate linkage. In preferred embodiments of UNC13A AONs described herein, all internucleoside linkages of the oligonucleotide are phosphorothioate linkages. Thus, in some embodiments, all of the nucleotide linkages of a UNC13A AON of any of SEQ ID NOs: 1-1264, SEQ ID NO: 2529-3792, SEQ ID NOs: 5066-5166, SEQ ID NOs: 5168-5202, SEQ ID NOs: 5209-5221, or SEQ ID NOs: 5235-5292 are phosphorothioate linkages. In some embodiments, one or more of the nucleotide linkages of a UNC13A AON of any of SEQ ID NOs: 1-1264, SEQ ID NO: 2529-3792, SEQ ID NOs: 5066-5166, SEQ ID NOs: 5168-5202, SEQ ID NOs: 5209-5221, or SEQ ID NOs: 5235-5292 are phosphorothioate linkages.

In various embodiments, nucleotide linkages of UNC13A AON described herein such as any of SEQ ID NOs: 1-1264, SEQ ID NO: 2529-3792, SEQ ID NOs: 5066-5166, SEQ ID NOs: 5168-5202, SEQ ID NOs: 5209-5221, or SEQ ID NOs: 5235-5292 include a mix of phosphodiester and phosphorothioate linkages.

In some embodiments, nucleoside linkages linking a base at position 3 of a UNC13A AON described herein are phosphodiester bonds. For example, the base at position 3 may be linked to each adjacent base (e.g., preceding base and succeeding base) through a phosphodiester bond. An example 25mer UNC13A AON with phosphodiester bonds linking the base at position 3 can be denoted as:

XXoDoXXXXXXXXXXXXXXXXXXXXXX

where “o” represents a phosphodiester bond and “D” represents the base at position 3. Any nucleobase in the AON can be a nucleobase analog.

In some embodiments, one of the nucleoside linkages linking a base at position 3 of a UNC13A AON described herein is a phosphodiester bond. For example, the base at position 3 may be linked to either the preceding base or the succeeding base through a phosphodiester bond. An example 25mer UNC13A AON with a phosphodiester bond linking the base at position 3 to a preceding base can be denoted as:

XXoDXXXXXXXXXXXXXXXXXXXXXX

where “o” represents a phosphodiester bond and “D” represents the base at position 3. Any nucleobase in the AON can be a nucleobase analog.

An example 25mer UNC13A AON with a phosphodiester bond linking the base at position 3 to a succeeding base can be denoted as:

XXDoXXXXXXXXXXXXXXXXXXXXXX

where “o” represents a phosphodiester bond and “D” represents the base at position 3. Any nucleobase in the AON can be a nucleobase analog.

In various embodiments, in addition to one of the nucleoside linkages linking a base at position 3 of a UNC13A AON described herein being a phosphodiester bond, the UNC13A AON further includes two spacers. The two spacers can be positioned in the UNC13A AON such that the UNC13A AON includes a segment with at most 7 linked nucleosides. An example 25mer UNC13A AON with two spacers and with a phosphodiester bond linking the base at position 3 to a preceding base can be denoted as:

XxoDS₁XXXXXXXXXS2XXXXXXXXXXX

where “S₁” represents a first spacer, “S₂” represents a second spacer, “o” represents a phosphodiester bond and “D” represents the base at position 3. Any nucleobase in the AON can be a nucleobase analog.

An example 25mer UNC13A AON with two spacers and with a phosphodiester bond linking the base at position 3 to a succeeding base can be denoted as:

XXDoXXXXXXXS₁XXXXXXXXXS2XXXX

where “S₁” represents a first spacer, “S₂” represents a second spacer, “o” represents a phosphodiester bond and “D” represents the base at position 3. Any nucleobase in the AON can be a nucleobase analog.

In some embodiments, nucleoside linkages linking a base at position 4 of a UNC13A AON described herein are phosphodiester bonds. For example, the base at position 4 may be linked to each adjacent base (e.g., preceding base and succeeding base) through a phosphodiester bond. An example 25mer UNC13A AON with phosphodiester bonds linking the base at position 4 can be denoted as:

XXXoDoXXXXXXXXXXXXXXXXXXXXX

where “o” represents a phosphodiester bond and “D” represents the base at position 4. Any nucleobase in the AON can be a nucleobase analog.

In some embodiments, one of the nucleoside linkages linking a base at position 4 of a UNC13A AON described herein is a phosphodiester bond. For example, the base at position 4 may be linked to either the preceding base or the succeeding base through a phosphodiester bond. An example 25mer UNC13A AON with a phosphodiester bond linking the base at position 4 to a preceding base can be denoted as:

XXXoDXXXXXXXXXXXXXXXXXXXXX

where “o” represents a phosphodiester bond and “D” represents the base at position 4. Any nucleobase in the AON can be a nucleobase analog.

An example 25mer UNC13A AON with a phosphodiester bond linking the base at position 4 to a succeeding base can be denoted as:

XXXDoXXXXXXXXXXXXXXXXXXXXX

where “o” represents a phosphodiester bond and “D” represents the base at position 4. Any nucleobase in the AON can be a nucleobase analog.

In some embodiments, nucleoside linkages linking both bases at position 3 and position 4 of a UNC13A AON described herein are phosphodiester bonds. For example, the base at position 3 may be linked to each adjacent base (e.g., preceding base and succeeding base) through a phosphodiester bond, and the base at position 4 may be linked to each adjacent base (e.g., preceding base and succeeding base) through a phosphodiester bond. An example 25mer UNC13A AON with phosphodiester bonds linking the bases at positions 3 and 4 can be denoted as:

XXoDoEoXXXXXXXXXXXXXXXXXXXXX

where “o” represents a phosphodiester bond, “D” represents the base at position 3, and “E” represents the base at position 4. In various embodiments, all other bases of the UNC13A AON are linked through phosphorothioate bonds. Any nucleobase in the AON can be a nucleobase analog.

In various embodiments, UNC13A AON described herein include one or more spacers and phosphodiester bonds are located relative to the one or more spacers. In some embodiments, the Y number of bases immediately preceding a spacer are linked through phosphodiester bonds. In various embodiments, Y is one, two, three, four, five, six, seven, eight, nine, ten, eleven, or twelve bases. In particular embodiments, Y is two bases. For example, if the spacer is located at position 15, the bases at positions 13 and 14 of the UNC13A AON are each linked to their respective adjacent bases through phosphodiester bonds. As described herein, the spacer can be located at various positions in the UNC13A AON and therefore, the 2 bases immediately preceding the spacer can vary within the UNC13A AON depending on where the spacer is situated.

In various embodiments, the UNC13A AON may include more than one spacer. In some embodiments, only one of the spacers has Y number of bases immediately preceding the spacer that are linked through phosphodiester bonds. In such embodiments, the other spacers are linked to respective preceding bases through phosphorothioate bonds. In various embodiments, two of the spacers have Y number of bases immediately preceding the spacers that are linked through phosphodiester bonds. In various embodiments, each of the spacers in the UNC13A AON have Y number of bases immediately preceding the spacers that are linked through phosphodiester bonds. In various embodiments, all other bases of the UNC13A AON are linked through phosphorothioate bonds.

In some embodiments, Y number of bases immediately preceding a spacer and Z number of bases immediately succeeding a spacer are linked through phosphodiester bonds. In various embodiments, Y is one, two, three, four, five, six, seven, eight, nine, ten, eleven, or twelve bases. In various embodiments, Z is one, two, three, four, five, six, seven, eight, nine, ten, eleven, or twelve bases. Y and Z can be independent of each other. In particular embodiments, Y is one base and Z is one base. For example, if the spacer is located at position 15, the bases at positions 14 and 16 of the UNC13A AON are each linked to their respective adjacent bases through phosphodiester bonds. To provide an example, such a UNC13A AON (e.g., 25mer) can be denoted as:

XXXXXXXXXXXXXoDoSoEoXXXXXXXXX

where “S” represents a spacer, “o” represents a phosphodiester bond, “D” represents a base immediately preceding the spacer, and “E” represents the base immediately succeeding the spacer. Any nucleobase in the AON can be a nucleobase analog.

As described herein, the spacer can be located at various positions in the UNC13A AON and therefore, the bases immediately preceding or immediately succeeding the spacer can vary within the UNC13A AON depending on where the spacer is situated.

In various embodiments, the UNC13A AON may include more than one spacer. In some embodiments, only one of the spacers has Y number of bases immediately preceding the spacer and Z number of bases immediately succeeding the spacer that are linked through phosphodiester bonds. In such embodiments, the other spacers of the UNC13A AON are linked to respective preceding and succeeding bases through phosphorothioate bonds. To provide an example, such a UNC13A AON (e.g., 25mer) can be denoted as:

XXXXoDoS1oEoXXXXXXXXXXXS2XXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

As another example, such a UNC13A AON (e.g., 25mer) can be denoted as:

XXXXXS1XXXXXXXXXXXoDoS₂oDoXXXXX

where “S₁” represents a first spacer, “S₂” represents a second spacer, “o” represents a phosphodiester bond, “D” represents a base immediately preceding the spacer, and “E” represents the base immediately succeeding the spacer. Any nucleobase in the AON can be a nucleobase analog.

In some embodiments, one of the spacers is linked to the immediately preceding base through a phosphodiester bond. For example, a UNC13A AON includes a first spacer that is linked to the immediately preceding base through a phosphodiester bond, which can be denoted as:

XXXXXXoS₁XXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a UNC13A AON includes a second spacer that is linked to the immediately preceding base through a phosphodiester bond, which can be denoted as:

XXXXXXS₁XXXXXXXXXXXoS₂XXXXXX

where “S₁” represents a first spacer, “S₂” represents a second spacer, “o” represents a phosphodiester bond. Any nucleobase in the AON can be a nucleobase analog.

In various embodiments, the UNC13A AON may be a AON variant (e.g., a 23mer, a 21mer, or a 19mer) where one of the spacers is linked to the immediately preceding base through a phosphodiester bond. For example, the UNC13A AON may be a 21mer with a first spacer that is linked to the immediately preceding base through a phosphodiester bond, which can be denoted as:

XXXXXXXoS₁XXXXXS₂XXXXXXX

where “S₁” represents a first spacer, “S₂” represents a second spacer, “o” represents a phosphodiester bond. Any nucleobase in the AON can be a nucleobase analog.

As another example, the UNC13A AON may be a 21mer with a second spacer that is linked to the immediately preceding base through a phosphodiester bond, which can be denoted as:

XXXXXXXS₁XXXXXoS₂XXXXXXX

where “S₁” represents a first spacer, “S₂” represents a second spacer, “o” represents a phosphodiester bond. Any nucleobase in the AON can be a nucleobase analog.

In some embodiments, the UNC13A AON may be a AON variant (e.g., a 23mer, a 21mer, or a 19mer) where one of the spacers is linked to the immediately preceding base through a phosphodiester bond and the immediately preceding base is further linked to the preceding base through a phosphodiester bond. An example 21mer UNC13A AON can be denoted as:

XXXEoDoS₁XXXXXXS₂XXXXXXX

where “S₁” represents a first spacer, “S₂” represents a second spacer, “o” represents a phosphodiester bond, “D” represents the base immediately preceding S₁ and “E” represents the base immediately preceding “D.” Any nucleobase in the AON can be a nucleobase analog.

As another example, a 21mer UNC13A AON can be denoted as:

XXXXXS₁XXXXEoDoS₂XXXXXXX

where “S₁” represents a first spacer, “S₂” represents a second spacer, “o” represents a phosphodiester bond, “D” represents the base immediately preceding S₂ and “E” represents the base immediately preceding “D.” Any nucleobase in the AON can be a nucleobase analog.

In some embodiments, the UNC13A AON may be a AON variant (e.g., a 23mer, a 21mer, or a 19mer) where a base that immediately precedes a first spacer is linked to another base through a phosphodiester bond. The base that immediately precedes the first spacer may be linked to the first spacer through a non-phosphodiester bond, such as a phosphorothioate bond. Additionally a second spacer is linked to an immediately preceding base through a phosphodiester bond. An example of a 21mer UNC13A AON can be denoted as:

XXXEoDS₁XXXXXXoS₂XXXXXXX

where “S₁” represents a first spacer, “S₂” represents a second spacer, “o” represents a phosphodiester bond, “D” represents the base immediately preceding S₁ and “E” represents the base immediately preceding “D.” Here, the base “D” is linked to the first spacer S₁ through a non-phosphodiester bond (e.g., phosphorothioate bond). Additionally, the base “D” is linked to base “E” through a phosphodiester bond. The second spacer S₂ is linked to an immediately preceding base through a phosphodiester bond. Any nucleobase in the AON can be a nucleobase analog.

Another example of such a 21mer UNC13A AON can be denoted as:

XXXXXoS₁XXXXEoDS₂XXXXXXX

where “S₁” represents a first spacer, “S₂” represents a second spacer, “o” represents a phosphodiester bond, “D” represents the base immediately preceding S₂ and “E” represents the base immediately preceding “D.” Here, the base “D” is linked to the second spacer S₂ through a non-phosphodiester bond (e.g., phosphorothioate bond). Additionally, the base “D” is linked to base “E” through a phosphodiester bond. The first spacer S₁ is linked to an immediately preceding base through a phosphodiester bond. Any nucleobase in the AON can be a nucleobase analog.

In some embodiments, one of the spacers is linked to the immediately succeeding base through a phosphodiester bond. For example, a UNC13A AON includes a first spacer that is linked to the immediately succeeding base through a phosphodiester bond, which can be denoted as:

XXXXXXS₁oXXXXXXXXXXXS₂XXXXXX

where “S₁” represents a first spacer, “S₂” represents a second spacer, “o” represents a phosphodiester bond. Any nucleobase in the AON can be a nucleobase analog.

As another example, a UNC13A AON includes a second spacer that is linked to the immediately succeeding base through a phosphodiester bond, which can be denoted as:

XXXXXXS₁XXXXXXXXXXXS₂oXXXXXX

where “S₁” represents a first spacer, “S₂” represents a second spacer, “o” represents a phosphodiester bond. Any nucleobase in the AON can be a nucleobase analog.

In various embodiments, the UNC13A AON may be a AON variant (e.g., a 23mer, a 21mer, or a 19mer) where one of the spacers is linked to the immediately succeeding base through a phosphodiester bond. For example, the UNC13A AON may be a 21mer with a first spacer that is linked to the immediately succeeding base through a phosphodiester bond, which can be denoted as:

XXXXXXXS₁oXXXXXS₂XXXXXXX

where “S₁” represents a first spacer, “S₂” represents a second spacer, “o” represents a phosphodiester bond. Any nucleobase in the AON can be a nucleobase analog.

As another example, the UNC13A AON may be a 21mer with a second spacer that is linked to the immediately succeeding base through a phosphodiester bond, which can be denoted as:

XXXXXXXS₁XXXXXS₂oXXXXXXX

where “S₁” represents a first spacer, “S₂” represents a second spacer, “o” represents a phosphodiester bond. Any nucleobase in the AON can be a nucleobase analog.

In various embodiments, two of the spacers have Y number of bases immediately preceding the spacers and Z number of bases immediately succeeding the spacers that are linked through phosphodiester bonds. In various embodiments, each of the spacers in the UNC13A AON have Y number of bases immediately preceding the spacers and Z number of bases immediately succeeding the spacers that are linked through phosphodiester bonds. An example of such a UNC13A AON (e.g., 25mer) can be denoted as:

XXXXoDoS₁oEoXXXXXXXXXXoFoS₂oHoXXXXX

where “S₁” represents a first spacer, “S₂” represents a second spacer, “o” represents a phosphodiester bond, “D” represents a base immediately preceding the first spacer, “E” represents the base immediately succeeding the first spacer, “F” represents a base immediately preceding the second spacer, and “H” represents the base immediately succeeding the second spacer. In various embodiments, all other bases of the UNC13A AON are linked through phosphorothioate bonds. Any nucleobase in the AON can be a nucleobase analog.

In various UNC13A AON includes two or more spacers and a range of bases located between the two spacers are linked through phosphodiester bonds. In various embodiments, the range of bases include two, three, four, five, six, or seven bases linked through phosphodiester bonds. In particular embodiments, the range of bases include two bases linked through phosphodiester bonds. In particular embodiments, the range of bases include four bases linked through phosphodiester bonds. In various embodiments, all other bases of the UNC13A AON are linked through phosphorothioate bonds. Any nucleobase in the AON can be a nucleobase analog.

In various embodiments, the range of bases linked through phosphodiester bonds are positioned Y number of bases succeeding the first spacer and Z number of preceding the second spacer. In various embodiments, Y is one, two, three, four, five, six, or seven bases. In various embodiments, Z is one, two, three, four, five, six, or seven bases. Y and Z can be independent on each other. Any nucleobase in the AON can be a nucleobase analog.

In particular embodiments, Y is five bases and Z is four bases. To provide an example, such a UNC13A AON (e.g., 25mer) can be denoted as:

XXXXXXXXS₁XXXXoDoEoFoHoXXXS₂XXXX

where “S₁” represents a first spacer, “S₂” represents a second spacer, and “o” represents a phosphodiester bond. The bases “D,” “E,” “F,” and “H” represent the range of bases that are linked through phosphodiester bonds. In this example, the range of bases is located five bases after the first spacer (e.g., D is positioned five bases after the first spacer) and the range of bases is located four bases preceding the second spacer (e.g., H is positioned four bases before the second spacer). Any nucleobase in the AON can be a nucleobase analog.

In particular embodiments, Y is four bases and Z is three bases. To provide an example, such a UNC13A AON (e.g., 23mer) can be denoted as:

XXXXXXXS₁XXXoDoEoXXS₂XXXXXXX

where “S₁” represents a first spacer, “S₂” represents a second spacer, and “o” represents a phosphodiester bond. The bases “D” and “E” represent the range of bases that are linked through phosphodiester bonds. In this example, the range of bases is located four bases after the first spacer (e.g., D is positioned four bases after the first spacer) and the range of bases is located three bases preceding the second spacer (e.g., E is positioned three bases before the second spacer). In various embodiments, the positions of the two spacers differ than shown above and therefore, the range of bases linked through phosphodiester bonds are differently positioned. In various embodiments, all other bases of the UNC13A AON are linked through phosphorothioate bonds. Any nucleobase in the AON can be a nucleobase analog.

In some embodiments, a disclosed UNC13A AON may have at least one modified nucleobase, e.g., 5-methylcytosine, and/or at least one methylphosphonate nucleotide, which is placed, for example, either at only one of the 5′ or 3′ ends or at both 5′ and 3′ ends or along the oligonucleotide sequence.

UNC13A AONs may include at least one modified sugar. For example, the sugar moiety of at least one nucleotide constituting the oligonucleotide is a ribose in which the 2′—OH group may be replaced by any one selected from the group consisting of OR, R, R′OR, SH, SR, NH₂, NR₂, N₃, CN, F, Cl, Br, and I (wherein R is an alkyl or aryl and R′ is an alkylene). Examples of a modified sugar moiety include a 2′-Ome modified sugar moiety, bicyclic sugar moiety, 2′-O-(2-methoxyethyl) (2′MOE or MOE), 2′-O—(N-methylacetamide), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

In some embodiments, UNC13A AONs comprise 2′OMe (e.g., a UNC13A AON comprising one or more 2′OMe modified sugar), 2′MOE or MOE (e.g., a UNC13A AON comprising one or more 2′MOE modified sugar), PNA (e.g., a UNC13A AON comprising one or more N-(2-aminoethyl)-glycine units linked by amide bonds or carbonyl methylene linkage as repeating units in place of a sugar-phosphate backbone), LNA (e.g., a UNC13A AON comprising one or more locked ribose, and can be a mixture of 2′-deoxy nucleotides or 2′OMe nucleotides), c-ET (e.g., a UNC13A AON comprising one or more cET sugar), cMOE (e.g., a UNC13A AON comprising one or more cMOE sugar), morpholino oligomer (e.g., a UNC13A AON comprising a backbone comprising one or more PMO), deoxy-2′-fluoro nucleoside (e.g., a UNC13A AON comprising one or more 2′-fluoro-β-D-arabinonucleoside), tcDNA (e.g., a UNC13A AON comprising one or more tcDNA modified sugar), ENA (e.g., a UNC13A AON comprising one or more ENA modified sugar), or HNA (e.g., a UNC13A AON comprising one or more HNA modified sugar). In some embodiments, a UNC13A AON comprises one or more phosphorothioate linkage, phosphodiester linkage, phosphotriester linkage, methylphosphonate linkage, phosphoramidate linkage, a phosphoramidothioate linkage, a thiophosphorodiamidate linkage, morpholino linkage, PNA linkage, or any combination of phosphorothioate linkage, phosphodiester linkage, a phosphotriester linkage, methylphosphonate linkage, phosphoramidate linkage, morpholino linkage, and PNA linkage. In some embodiments, a UNC13A AON comprises one or more phosphorothioate linkage, phosphodiester linkage, or a combination of phosphorothioate and phosphodiester linkages.

In some embodiments, UNC13A AONs with a sequence of any one of SEQ ID NOs: 1-1264, SEQ ID NOs: 2529-3792, SEQ ID NOs: 5066-5166, SEQ ID NOs: 5168-5202, SEQ ID NOs: 5209-5221, or SEQ ID NOs: 5235-5292 is a chirally controlled oligonucleotide, such as a chirally controlled oligonucleotide described in any of U.S. Pat. Nos. 9,982,257, 10,590,413, 10,724,035, 10,450,568, and PCT Publication No. WO2019200185, each of which is hereby incorporated by reference in its entirety.

For example, a UNC13A AON with a sequence of any one of SEQ ID NOs: 1-1264, SEQ ID NOs: 2529-3792, SEQ ID NOs: 5066-5166, SEQ ID NOs: 5168-5202, SEQ ID NOs: 5209-5221, or SEQ ID NOs: 5235-5292 is a chirally controlled oligonucleotide comprising a plurality of oligonucleotides of at least one type, wherein each type is defined by: 1) base sequence; 2) pattern of backbone linkages; 3) pattern of backbone chiral centers; and 4) pattern of backbone X-moieties (—X-L-R¹); wherein: the oligonucleotides of the at least one type comprise one or more phosphorothioate triester internucleotidic linkages and one or more phosphate diester linkage; the oligonucleotides of the at least one type comprise at least two consecutive modified internucleotidic linkages; and oligonucleotides of the at least one oligonucleotide type comprise one or more modified internucleotidic linkages independently having the structure of:

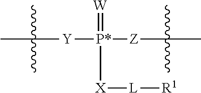

wherein: P* is an asymmetric phosphorus atom and is either Rp or Sp;
W is O, S or Se; each of X, Y and Z is independently —O—, —S—, —N(-L-R¹)—, or L; L is a covalent bond or an optionally substituted, linear or branched C₁-C₅₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; R¹ is halogen, R, or an optionally substituted C₁-C₁₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—; each R′ is independently —R, —C(O)R, —CO₂R, or —SO₂R, or: two R′ on the same nitrogen are taken together with their intervening atoms to form an optionally substituted heterocyclic or heteroaryl ring, or two R′ on the same carbon are taken together with their intervening atoms to form an optionally substituted aryl, carbocyclic, heterocyclic, or heteroaryl ring; -Cy- is an optionally substituted bivalent ring selected from phenylene, carbocyclylene, arylene, heteroarylene, or heterocyclylene; each R is independently hydrogen, or an optionally substituted group selected from C₁-C₆ aliphatic, phenyl, carbocyclyl, aryl, heteroaryl, or heterocyclyl; and each

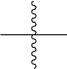

independently represents a connection to a nucleoside. In some embodiments, a UNC13A AON with a sequence of any one of SEQ ID NOs: 1-1264, SEQ ID NOs: 2529-3792, SEQ ID NOs: 5066-5166, SEQ ID NOs: 5168-5202, SEQ ID NOs: 5209-5221, or SEQ ID NOs: 5235-5292 is a chirally controlled oligonucleotide comprising certain chemical modifications (e.g., 2′F (2′ Fluoro, which contains a fluorine molecule at the 2′ ribose position (instead of 2′-hydroxyl group in an RNA monomer)), 2′-OMe, phosphorothioate linkages, lipid conjugation, etc.), as described in U.S. Pat. No. 10,450,568.

### Motor Neuron Diseases

Motor neuron diseases are a group of diseases characterized by loss of function of motor neurons that coordinate voluntary movement of muscles by the brain. Motor neuron diseases may affect upper and/or lower motor neurons, and may have sporadic or familial origins. Motor neuron diseases include amyotrophic lateral sclerosis (ALS or Lou Gehrig's disease), progressive bulbar palsy, pseudobulbar palsy, progressive muscular atrophy, primary lateral sclerosis, spinal muscular atrophy, post-polio syndrome, and ALS with frontotemporal dementia.

Symptoms of motor neuron diseases include muscle decay or weakening, muscle pain, spasms, slurred speech, difficulty swallowing, loss of muscle control, joint pain, stiff limbs, difficulty breathing, drooling, and complete loss of muscle control, including over basic functions such as breathing, swallowing, eating, speaking, and limb movement. These symptoms are also sometimes accompanied by depression, loss of memory, difficulty with planning, language deficits, altered behavior, and difficulty assessing spatial relationships and/or changes in personality.

Motor neuron diseases can be assessed and diagnosed by a clinician of skill, for example, a neurologist, using various tools and tests. For example, the presence or risk of developing a motor neuron disease can be assessed or diagnosed using blood and urine tests (for example, tests that assay for the presence of creatinine kinase), magnetic resonance imaging (MRI), electromyography (EMG), nerve conduction study (NCS), spinal tap, lumbar puncture, and/or muscle biopsy. Motor neuron diseases can be diagnosed with the aid of a physical exam and/or a neurological exam to assess motor and sensory skills, nerve function, hearing and speech, vision, coordination and balance, mental status, and changes in mood or behavior.

### Amyotrophic Lateral Sclerosis

ALS is a progressive motor neuron disease that disrupts signals to all voluntary muscles. ALS results in atrophy of both upper and lower motor neurons. Symptoms of ALS include weakening and wasting of the bulbar muscles, general and bilateral loss of strength, spasticity, muscle spasms, muscle cramps, fasciculations, slurred speech, and difficulty breathing or loss of ability to breathe. Some individuals with ALS also suffer from cognitive decline. At the molecular level, ALS is characterized by protein and RNA aggregates in the cytoplasm of motor neurons, including aggregates of the RNA-binding protein TDP43.

ALS is most common in males above 40 years of age, although it can also occur in women and children. Risk of ALS is also heightened in individuals who smoke, are exposed to chemicals such as lead, or who have served in the military. Most instances of ALS are sporadic, while only about 10% of cases are familial. Causes of ALS include sporadic or inherited genetic mutations, high levels of glutamate, protein mishandling. Genetic mutations associated with ALS include mutations in the genes SOD1, C9orf72, TARDBP, FUS, ANG, ATXN2, CHCHD10, CHMP2B, DCTN1, ErbB4, FIG4, HNRPA1, MATR3, NEFH, OPTN, PFN1, PRPH, SETX, SIGMAR1, SMN1, SPG11, SQSTM1, TBK1, TRPM7, TUBA4A, UBQLN2, VAPB, and VCP.

### Frontotemporal Dementia

Frontotemporal dementia (FTD) is a form of dementia that affects the frontal and temporal lobes of the brain. FTD includes frontotemporal lobar degeneration (FTLD). It has an earlier average age of onset than Alzheimer's disease—40 years of age. Symptoms of FTD include extreme changes in behavior and personality, speech and language problems, and movement-related symptoms such as tremor, rigidity, muscle spasm, weakness, and difficulty swallowing. Subtypes of FTD include behavior variant frontotemporal dementia (bvFTD), characterized by changes in personality and behavior, and primary progressive aphasia (PPA), which affects language skills, speaking, writing and comprehension. FTD is associated with tau protein accumulation (Pick bodies) and altered TDP43 function. About 30% of cases of FTD are familial, and no other risk factors other than family history of the disease are known. Genetic mutations associated with FTD include mutations in the genes C9orf72, Progranulin (GRN), microtubule-associated protein tau (MAPT), UBQLN2, VPC, CHMP2B, TARDBP, FUS, ITM2B, CHCHD10, SQSTM1, PSEN1, PSEN2, CTSF, CYP27A1, TBK1 and TBP.

Amyotrophic Lateral Sclerosis with Frontotemporal Dementia

Amyotrophic lateral sclerosis with frontotemporal dementia (ALS with FTD) is a clinical syndrome in which FTD and ALS occur in the same individual. Interestingly, mutations in C9orf72 are the most common cause of familial forms of ALS and FTD. Additionally, mutations in TBK1, VCP, SQSTM1, UBQLN2 and CHMP2B are also associated with ALS with FTD. Symptoms of ALS with FTD include dramatic changes in personality, as well as muscle weakness, muscle atrophy, fasciculations, spasticity, dysarthria, dysphagia, and degeneration of the spinal cord, motor neurons, and frontal and temporal lobes of the brain. At the molecular level, ALS with FTD is characterized by the accumulation of TDP-43 and/or FUS proteins. TBK1 mutations are associated with ALS, FTD, and ALS with FTD.

### Limbic-Predominant Age-Related TDP-43 Encephalopathy (LATE)

Limbic-predominant age-related TDP-43 encephalopathy (LATE) is characterized by accumulation of misfolded TDP-43 protein in the brain, specifically in the limbic system. LATE is a neurological disorder that typically manifests in older patients (e.g., greater than 80 years old). LATE can be a diagnosis for dementia and LATE often mimics the symptoms of Alzheimer's Disease including memory loss, confusion, and mood changes.

### Methods of Treatment

The disclosure contemplates, in part, treating neurological diseases including any of amyotrophic lateral sclerosis (ALS), frontotemporal dementia (FTD), Alzheimer's disease (AD), Parkinson's disease (PD), Huntington's disease, progressive supranuclear palsy (PSP), brain trauma, spinal cord injury, corticobasal degeneration (CBD), Limbic-predominant age-related TDP-43 encephalopathy (LATE), epilepsy, Cerebral Age-Related TDP-43 With Sclerosis (CARTS), facial onset sensory and motor neuronopathy, Guam Parkinson-dementia complex, multisystem proteinopathy, CTE, and synaptic diseases like autism) in a patient in need thereof comprising administering a UNC13A AON. In some embodiments, provided herein are methods for treatment of a neurological disease in a patient in need thereof, comprising administering a disclosed UNC13A AON. In some embodiments of the disclosure, an effective amount of a disclosed UNC13A oligonucleotide may be administered to a patient in need thereof to treat a neurological disease, and/or to increase, restore, or stabilize expression of UNC13A mRNA that is capable of translation to produce a functional UNC13A protein, thereby increase, restore, or stabilize UNC13A activity and/or function.

In some embodiments, treating a neurological disease comprises at least ameliorating or reducing one symptom associated with the neurological disease (for example, reducing muscle weakness in a patient with ALS). Methods of treating a neurological disease (for example, ALS, FTD, or ALS with FTD) in a patient suffering therefrom are provided, that include administering a disclosed UNC13A AON. In some embodiments, methods of slowing the progression of a neurological disease, for example, a motor neuron disease, are provided.

Provided herein are methods of treating, reducing the risk of developing, or delaying the onset of a neurological disease in a subject in need thereof comprising administering a disclosed UNC13A AON. The methods include for example, treating a subject at risk of developing a neurological disease; e.g., administering to the subject an effective amount of a disclosed UNC13A AON. Neurological diseases that can be treated in this manner include motor neuron diseases, ALS, FTD, ALS with FTD, progressive bulbar palsy, pseudobulbar palsy, progressive muscular atrophy, primary lateral sclerosis, spinal muscular atrophy, and post-polio syndrome.

Methods of preventing or treating neurological diseases (for example, PD, ALS, FTD, and ALS with FTD) form part of this disclosure. Such methods may comprise administering to a patient in need thereof or a patient at risk, a pharmaceutical preparation comprising a UNC13A AON disclosed herein. For example, a method of preventing or treating a neurological disease is provided comprising administering to a patient in need thereof a UNC13A AON disclosed herein.

Patients treated using an above method may experience an increase, restoration of, or stabilization of UNC13A mRNA expression, which is capable of translation to produce a functional UNC13A protein, of at least about 5%, 10%, 20%, 30%, 40% or even 50%, thereby increase, restore, or stabilize UNC13A activity and/or function in a target cell (for example, a motor neuron) after administering a UNC13A oligonucleotide e.g. after 1 day, 2 days, 1 week, 2 weeks, 3 weeks, 4 weeks, 5 weeks, 6 weeks, 7 weeks, 8 weeks, 1 month, 2 months, 3, months, 4 months, 5, months, or 6 months or more. In some embodiments, administering such a UNC13A oligonucleotide may be on, e.g., at least a daily basis. The UNC13A oligonucleotide may be administered orally. In some embodiments, the UNC13A oligonucleotide is administered intrathecally, intrathalamically, or intracistemally. For example, in an embodiment described herein, a UNC13A oligonucleotide is administered intrathecally, intrathalamically or intracisternally about every 3 months. The delay or amelioration of clinical manifestation of a neurological disease in a patient as a consequence of administering a UNC13A oligonucleotide disclosed here may be at least e.g., 6 months, 1 year, 18 months or even 2 years or more as compared to a patient who is not administered a UNC13A oligonucleotide, such as one disclosed herein.

UNC13A oligonucleotides can be used alone or in combination with each other whereby at least two UNC13A oligonucleotides are used together in a single composition or as part of a treatment regimen. UNC13A oligonucleotides may also be used in combination with other drugs or AON for treating neurological diseases or conditions.

In various embodiments, disclosed herein is a method for treating amyotrophic lateral sclerosis (ALS) in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs: 5066-5166, SEQ ID NOs: 5168-5202, SEQ ID NOs: 5209-5221, or SEQ ID NOs: 5235-5292, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, disclosed herein is a method for treating frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs: 5066-5166, SEQ ID NOs: 5168-5202, SEQ ID NOs: 5209-5221, or SEQ ID NOs: 5235-5292, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

In various embodiments, disclosed herein is a method for treating amyotrophic lateral sclerosis (ALS) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs: 5066-5166, SEQ ID NOs: 5168-5202, SEQ ID NOs: 5209-5221, or SEQ ID NOs: 5235-5292, or a pharmaceutically acceptable salt thereof; wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally wherein the oligonucleotide further comprises a spacer.

### Treatment and Evaluation

A patient, as described herein, refers to any animal at risk for, suffering from or diagnosed with a neurological disease, including, but not limited to, mammals, primates, and humans. In certain embodiments, the patient may be a non-human mammal such as, for example, a cat, a dog, or a horse. A patient may be an individual diagnosed with a high risk of developing a neurological disease, someone who has been diagnosed with a neurological disease, someone who previously suffered from a neurological disease, or an individual evaluated for symptoms or indications of a neurological disease, for example, any of the signs or symptoms associated with neurological diseases such as: amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), TDP43 proteinopathies (e.g., chronic traumatic encephalopathy, Perry Syndrome, Dementia with Lewy body in association with Alzheimer's disease, Parkinson's disease with or without dementia, and Limbic-predominant age-related TDP-43 encephalopathy (LATE)), epilepsy, Cerebral Age-Related TDP-43 With Sclerosis (CARTS), facial onset sensory and motor neuronopathy, Guam Parkinson-dementia complex, multisystem proteinopathy, CTE, and synaptic diseases like autism.

“A patient in need,” as used herein, refers to a patient suffering from any of the symptoms or manifestations of a neurological disease, a patient who may suffer from any of the symptoms or manifestations of a neurological disease, or any patient who might benefit from a method of the disclosure for treating a neurological disease. A patient in need may include a patient who is diagnosed with a risk of developing a neurological disease, a patient who has suffered from a neurological disease in the past, or a patient who has previously been treated for a neurological disease.

“Effective amount,” as used herein, refers to the amount of an agent that is sufficient to at least partially treat a condition when administered to a patient. The therapeutically effective amount will vary depending on the severity of the condition, the route of administration of the component, and the age, weight, etc. of the patient being treated. Accordingly, an effective amount of a disclosed UNC13A oligonucleotide is the amount of the UNC13A oligonucleotide necessary to treat a neurological disease in a patient such that administration of the agent prevents a neurological disease from occurring in a subject, prevents neurological disease progression (e.g., prevents the onset or increased severity of symptoms of the neurological such as muscle weakening, spasms, or fasciculation), or relieves or completely ameliorates all associated symptoms of a neurological disease, i.e. causes regression of the disease.

Efficacy of treatment may be evaluated by means of evaluation of gross symptoms associated with a neurological disease, analysis of tissue histology, biochemical assay, imaging methods such as, for example, magnetic resonance imaging, or other known methods. For instance, efficacy of treatment may be evaluated by analyzing gross symptoms of the disease such as changes in muscle strength and control or other aspects of gross pathology associated with a neurological disease following administration, to a patient suffering from a neurological disease, a disclosed UNC13A oligonucleotide.

Efficacy of treatment may also be evaluated at the tissue or cellular level, for example, by means of obtaining a tissue biopsy (e.g., a brain, spinal, muscle, motor neuron tissue biopsy, or olfactory neurosphere cell biopsy) and evaluating gross tissue or cell morphology or staining properties. Biochemical assays that examine protein or RNA expression may also be used to evaluate efficacy of treatment. For instance, one may evaluate levels of a protein or gene product indicative of a neurological disease, in dissociated cells or non-dissociated tissue via immunocytochemical, immunohistochemical, Western blotting, or Northern blotting methods, or methods useful for evaluating RNA levels such as quantitative or semi-quantitative polymerase chain (e.g., digital PCR (DigitalPCR, dPCR, or dePCR), qPCR etc.) reaction. One may also evaluate the presence or level of expression of useful biomarkers (e.g., neurofilament light (0), neurofilament heavy (NEFH), TDP-43 or p 75 extracellular domain (p75^{ECD})) found in spinal cord fluid, cerebrospinal fluid, extracellular vesicles (for example, exosome-like cerebrospinal fluid extracellular vesicles (“CSF exosomes”), such as those described in Welton et al., (2017) “Cerebrospinal fluid extracellular vesicle enrichment for protein biomarker discovery in neurological disease; multiple sclerosis” J Extracell Vesicles, 6(1):1-10; and Street et al., (2012) “Identification and proteomic profiling of exosomes in human cerebrospinal fluid” J Transl. Med., 10:5), urine, fecal matter, lymphatic fluid, blood, plasma, or serum to evaluate disease state and efficacy of treatment. One may also evaluate the presence or level of expression of useful biomarkers found in the plasma, neuronal extracellular vesicles/exosomes. Additional measurements of efficacy may include strength duration time constant (SDTC), short interval cortical inhibition (SICI), dynamometry, accurate test of limb isometric strength (ATLIS), compound muscle action potential (CMAP), and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in C₉ORF72-associated amyotrophic lateral sclerosis (c9ALS) patients. CSF pNFH as a prognostic biomarker for clinical trials, which will increase the likelihood of successfully developing a treatment for c9ALS.

In evaluating efficacy of treatment, suitable controls may be chosen to ensure a valid assessment. For instance, one can compare symptoms evaluated in a patient with a neurological disease following administration of a disclosed UNC13A oligonucleotide to those symptoms in the same patient prior to treatment or at an earlier point in the course of treatment or in another patient not diagnosed with the neurological disease. Alternatively, one may compare the results of biochemical or histological analysis of tissue following administration of a disclosed UNC13A oligonucleotide with those of tissue from the same patient or from an individual not diagnosed with the neurological disease or from the same patient prior to administration of the UNC13A oligonucleotide. Additionally, one may compare blood, plasma, serum, cell, urine, lymphatic fluid, spinal cord fluid, cerebrospinal fluid, or fecal samples following administration of the UNC13A oligonucleotide with comparable samples from an individual not diagnosed with the neurological disease or from the same patient prior to administration of the UNC13A oligonucleotide. In some embodiments one may compare extracellular vesicles (for example CSF exosomes), following administration of the UNC13A oligonucleotide with extracellular vesicles from an individual not diagnosed with the neurological disease or from the same patient prior to administration of the UNC13A oligonucleotide.

Validation of UNC13A oligonucleotides may be determined by direct or indirect assessment of UNC13A expression levels or activity. For instance, biochemical assays that measure UNC13A protein or RNA expression may be used to evaluate overall effect on UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065 or SEQ ID NOs: 5206-5208. For instance, one may measure UNC13A protein levels in cells or tissue by Western blot to evaluate overall UNC13A levels. One may also measure UNC13A mRNA levels by means of Northern blot or quantitative polymerase chain reaction to determine overall effect on UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065 or SEQ ID NOs: 5206-5208. One may also evaluate UNC13A protein levels or levels of another protein indicative of UNC13A signaling activity in dissociated cells, non-dissociated tissue, extracellular vesicles (for example, CSF exosomes), blood, serum, or fecal matter via immunocytochemical or immunohistochemical methods.

Modulation of expression levels of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065 or SEQ ID NOs: 5206-5208 may also be evaluated indirectly by measuring parameters such as autophagy, endocytosis, protein aggregation, and the presence or level of expression of useful biomarkers (e.g., neurofilament light (NEFL), neurofilament heavy (NEFH), TDP-43, or p75^{ECD} found in plasma, spinal cord fluid, cerebrospinal fluid, extracellular vesicles (for example, CSF exosomes), blood, urine, lymphatic fluid, fecal matter, or tissue to evaluate the modulation of expression of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065 or SEQ ID NOs: 5206-5208. Modulation of expression levels of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NOs: 5057-5065 or SEQ ID NOs: 5206-5208 may also be evaluated indirectly by measuring parameters such as autophagy, endocytosis, protein aggregation, and the presence or level of expression of physiological biomarkers such as compound muscle action potential (CMAP). Additional measurements may include strength duration time constant (SDTC), short interval cortical inhibition (SICI), dynamometry, accurate test of limb isometric strength (ATLIS), compound muscle action potential, and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in c9ALS patients. CSF pNFH as a prognostic biomarker for clinical trials, which will increase the likelihood of successfully developing a treatment for c9ALS.

The disclosure also provides methods of restoring expression of full length UNC13A transcripts in cells of a patient suffering from a neurological disease. Full length UNC13A transcripts may be restored in any cell in which UNC13A expression or activity occurs, including cells of the nervous system (including the central nervous system (e.g., spinal cord or brain), the peripheral nervous system, motor neurons, glial cells, astrocytes, oligodendrocytes, microglia, the brain, the brain stem, the frontal lobes, the temporal lobes, the spinal cord), the musculoskeletal system, spinal fluid, and cerebrospinal fluid. Cells of the musculoskeletal system include skeletal muscle cells (e.g., myocytes). Motor neurons include upper motor neurons and lower motor neurons.

### Pharmaceutical Compositions and Routes of Administration

The present disclosure also provides methods for treating a neurological disease via administration of a pharmaceutical composition comprising a disclosed UNC13A oligonucleotide. In another aspect, the disclosure provides a pharmaceutical composition for use in treating a neurological disease. The pharmaceutical composition may be comprised of a disclosed UNC13A oligonucleotide, and a pharmaceutically acceptable carrier. As used herein the term “pharmaceutical composition” means, for example, a mixture containing a specified amount of a therapeutic compound, e.g., a therapeutically effective amount, of a therapeutic compound in a pharmaceutically acceptable carrier to be administered to a mammal, e.g., a human, in order to treat a neurological disease. In some embodiments, described herein are pharmaceutical compositions comprising a disclosed UNC13A oligonucleotide, and a pharmaceutically acceptable carrier. In another aspect, the disclosure provides use of a disclosed UNC13A oligonucleotide in the manufacture of a medicament for treating a neurological disease. “Medicament,” as used herein, has essentially the same meaning as the term “pharmaceutical composition.”

As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art. In one embodiment the pharmaceutical composition is administered orally and includes an enteric coating suitable for regulating the site of absorption of the encapsulated substances within the digestive system or gut. For example, an enteric coating can include an ethylacrylate-methacrylic acid copolymer.

In one embodiment, a disclosed UNC13A oligonucleotide and any pharmaceutical composition thereof may be administered by one or several routes, including topically, intrathecally, intrathalamically, intracistemally, intracerebroventricularly, parenterally, orally, rectally, buccally, sublingually, vaginally, pulmonarily, intratracheally, intranasally, transdermally, or intraduodenally. The term parenteral as used herein includes subcutaneous injections, intrapancreatic administration, intravenous, intracistemal, intracerebroventricular, intrathecal, intrathalamic, intramuscular, intraperitoneal, intrasternal injection or infusion techniques. For example, a disclosed UNC13A oligonucleotide may be administered subcutaneously to a subject. In another example, a disclosed UNC13A oligonucleotide may be administered orally to a subject. In another example, a disclosed UNC13A oligonucleotide may be administered directly to the nervous system, or specific regions or cells of the nervous system (e.g., the brain, brain stem, lower motor neurons, spinal cord, upper motor neurons) via parenteral administration, for example, a disclosed UNC13A oligonucleotide may be administered intrathecally, intrathalamically intracisternally, or intracerebroventricularly.

In various embodiments, a UNC13A oligonucleotide, for example a UNC13A AON, can be exposed to calcium-containing buffers prior to administration. Such calcium-containing buffers can mitigate toxicity adverse effects of the UNC13A oligonucleotide. Further details of exposing an example antisense oligonucleotide to calcium-containing buffers is described in Moazami, et al., Quantifying and Mitigating Motor Phenotypes Induced by Antisense Oligonucleotides in the Central Nervous System, bioRxiv 2021.02.14.431096, which is hereby incorporated by reference in its entirety.

In some embodiments, a UNC13A oligonucleotide, for example a UNC13A AON, can be encapsulated in a nanoparticle coating. It is believed that nanoparticle encapsulation prevents AON degradation and enhances cellular uptake. For example, in some embodiments a UNC13A oligonucleotide is encapsulated in a coating of a cationic polymer, for example, a synthetic polymer (e.g., poly-L-lysine, polyamidoamine, a poly(β-amino ester), and polyethyleneimine) or a naturally occurring polymer (e.g., chitosan and a protamine). In some embodiments, a UNC13A oligonucleotide is encapsulated in a lipid or lipid-like material, for example, a cationic lipid, a cationic lipid-like material, or an ionizable lipid that is positively charged only at an acidic pH. An example of a lipid nanoparticle nucleotide therapy includes Exicure's XCUR—FXN, a lipid-nanoparticle spherical nucleic acid (SNA)-based therapeutic candidate. For example, in some embodiments, a UNC13A oligonucleotide is encapsulated in a lipid nanoparticle that includes hydrophobic moieties, e.g., cholesterol and/or a polyethylene glycol (PEG) lipid.

In various embodiments, a pharmaceutical composition comprising a disclosed UNC13A oligonucleotide may further comprise a bolaamphiphilic compound. Example bolaamphiphilic compounds are described in WO2014039493A1, WO2014039500A1, WO2014039502A1, WO2014039503A1, and WO2014039504A1, each of which is hereby incorporated by reference in its entirety. In particular embodiments, a bolaamphiphilic compound is a compound according to formula I: HG² L¹ HG¹, or a pharmaceutically acceptable salt, solvate, hydrate, prodrug, stereoisomer, tautomer, isotopic variant, or N-oxide thereof, or a combination thereof; wherein: each HG¹ and HG² is independently a hydrophilic head group; and L¹ is alkylene, alkenyl, heteroalkylene, or heteroalkenyl linker; unsubstituted or substituted with C1-C20 alkyl, hydroxyl, or oxo.

In one embodiment, with respect to the bolaamphiphilic compound of formula I, the bolaamphiphilic compound is a compound according to formula II, III, IV, V, or VI:

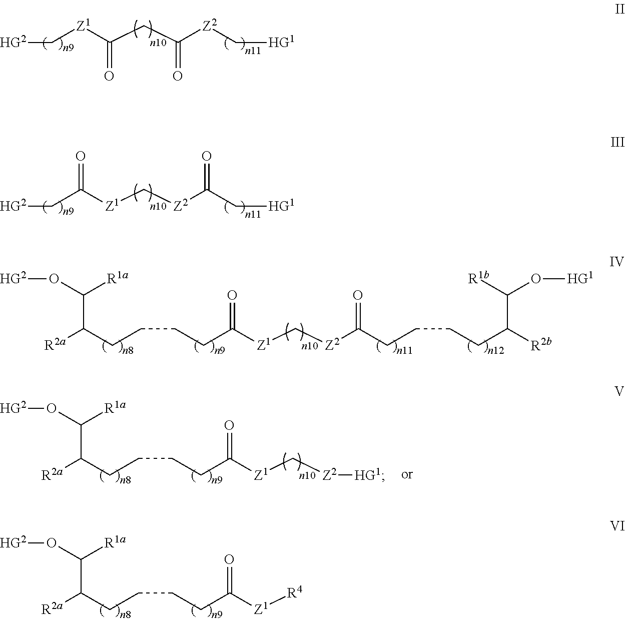

or a pharmaceutically acceptable salt, solvate, hydrate, prodrug, stereoisomer, tautomer, isotopic variant, or N-oxide thereof, or a combination thereof, wherein:
each HG¹ and HG² is independently a hydrophilic head group;
each Z¹ and Z² is independently —C(R³)₂—, —N(R³)— or —O—;
each R^{1a}, R^{1b}, R³, and R⁴ is independently H or Ci-C₈ alkyl;
each R^{2a} and R^{2b} is independently H, Ci-C₈ alkyl, OH, alkoxy, or O-HG¹ or O-HG²; each
n8, n9, n11, and n12 is independently an integer from 1-20;
n10 is an integer from 2-20; and
each dotted bond is independently a single or a double bond.

In one embodiment, with respect to the bolaamphiphilic compound of formula I, II, III, IV, V, or VI, each HG¹ and HG² is independently selected from:

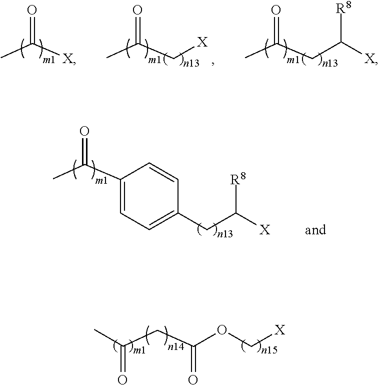

wherein: X is —NR^{5a}R^{5b}, or —N⁺R^{5a}R^{5b}R^{5c}; each R^{5a}, and R^{5b} is independently H or substituted or unsubstituted C₁-C₂₀ alkyl or R^{5a} and R^{5b} may join together to form an N containing substituted or unsubstituted heteroaryl, or substituted or unsubstituted heterocyclyl; each R^{5c} is independently substituted or unsubstituted C₁-C₂₀ alkyl; each R⁸ is independently H, substituted or unsubstituted C₁-C₂₀ alkyl, alkoxy, or carboxy; ml is 0 or 1; and each n13, n14, and n15 is independently an integer from 1-20.

In various embodiments, pharmaceutical compositions disclosed herein comprise complexes between bolaamphiphiles and pharmacologically or biologically active compounds (e.g., an UNC13A oligonucleotide disclosed herein). In various embodiments, the pharmaceutical compositions disclosed herein comprise a bolaamphiphile vesicle complexes comprising one or more bolaamphiphilic compounds and the biologically active compound is an oligonucleotide (e.g., an UNC13A oligonucleotide disclosed herein).

Pharmaceutical compositions containing a disclosed UNC13A oligonucleotide, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18^{th} ed. (Mack Publishing Company, 1990).

Pharmaceutical formulations, in some embodiments, are sterile. Sterilization can be accomplished, for example, by filtration through sterile filtration membranes. Where the composition is lyophilized, filter sterilization can be conducted prior to or following lyophilization and reconstitution.

### Parenteral Administration

The pharmaceutical compositions of the disclosure can be formulated for parenteral administration, e.g., formulated for injection via the intravenous, intracisternal, intracerebroventricular, intramuscular, subcutaneous, intrathecal, intrathalamic, intralesional, or intraperitoneal routes. The preparation of an aqueous composition, such as an aqueous pharmaceutical composition containing a disclosed UNC13A oligonucleotide, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including normal saline, artificial cerebrospinal fluid, sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In one embodiment, a disclosed UNC13A antisense oligonucleotide may be suspended in a carrier fluid comprising 1% (w/v) sodium carboxymethylcellulose and 0.1% (v/v) TWEEN™ 80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating a disclosed UNC13A antisense oligonucleotide in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of the disclosed oligonucleotide to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and for example, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfite, sodium metabisulfite, sodium thiosulfate, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose, petrolatum, polyethylene glycol, polyvinyl alcohol, polyvinylpyrrolidone, carboxymethylcellulose and the like.

### Oral Administration

In some embodiments, contemplated herein are compositions suitable for oral delivery of a disclosed UNC13A oligonucleotide, e.g., tablets that include an enteric coating, e.g., a gastro-resistant coating, such that the compositions may deliver a UNC13A oligonucleotide to, e.g., the gastrointestinal tract of a patient.

For example, a tablet for oral administration is provided that comprises granules (e.g., is at least partially formed from granules) that include a disclosed UNC13A oligonucleotide, e.g., a UNC13A oligonucleotide represented by any SEQ ID NOs: 1-1264, SEQ ID NO: 2529-3792, SEQ ID NOs: 5066-5166, SEQ ID NOs: 5168-5202, SEQ ID NOs: 5209-5221, or SEQ ID NOs: 5235-5292 that targets a UNC13A transcript comprising a sequence that shares at least 90% (e.g., 90%, 91%, 92%, 93%, 94%, 95%, 96%, 97%, 98%, 99%, or 100%) identity to any one of SEQ ID NO: 5057-5065 or SEQ ID NOs: 5206-5208, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed UNC13A oligonucleotide, e.g., a UNC13A oligonucleotide represented by any of SEQ ID NOs: 1-1264, SEQ ID NO: 2529-3792, SEQ ID NOs: 5066-5166, SEQ ID NOs: 5168-5202, SEQ ID NOs: 5209-5221, or SEQ ID NOs: 5235-5292 that targets a UNC13A transcript comprising a sequence that shares at least 90% (e.g., 90%, 91%, 92%, 93%, 94%, 95%, 96%, 97%, 98%, 99%, or 100%) identity to any one of SEQ ID NO: 5057-5065 or SEQ ID NOs: 5206-5208, and a pharmaceutically acceptable salt. In some embodiments, contemplated pharmaceutical formulations include an intra-granular phase that includes a disclosed UNC13A oligonucleotide, e.g., a UNC13A oligonucleotide represented by any of SEQ ID NOs: 1-1264, SEQ ID NO: 2529-3792, SEQ ID NOs: 5066-5166, SEQ ID NOs: 5168-5202, SEQ ID NOs: 5209-5221, or SEQ ID NOs: 5235-5292 that targets a UNC13A transcript comprising a sequence that shares at least 90% (e.g., 90%, 91%, 92%, 93%, 94%, 95%, 96%, 97%, 98%, 99%, or 100%) identity to any one of SEQ ID NO: 5057-5065 or SEQ ID NOs: 5206-5208, and a pharmaceutically acceptable filler. For example, a disclosed UNC13A oligonucleotide and a filler may be blended together, optionally, with other excipients, and formed into granules. In some embodiments, the intragranular phase may be formed using wet granulation, e.g., a liquid (e.g., water) is added to the blended UNC13A oligonucleotide and filler, and then the combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include an intragranular phase and/or an extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated formulations, e.g., that include an intragranular phase and/or an extragranular phase, may include a disintegrant such as but not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated formulation includes an intra-granular phase comprising a disclosed UNC13A oligonucleotide and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a contemplated formulation may include a lubricant, e.g. an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings may include a polymer that disintegrates at different rates according to pH. Enteric coatings may include for example, cellulose acetate phthalate, methyl acrylate-methacrylic acid copolymers, cellulose acetate succinate, hydroxypropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE®, Eudragit® grades. In some embodiments, an enteric coating may comprise about 5% to about 10%, about 5% to about 20%, 8% to about 15%, about 8% to about 20%, about 10% to about 20%, or about 12% to about 20%, or about 18% of a contemplated tablet by weight. For example, enteric coatings may include an ethylacrylate-methacrylic acid copolymer.

For example, in a contemplated embodiment, a tablet is provided that comprises or consists essentially of about 0.5% to about 70%, e.g., about 0.5% to about 10%, or about 1% to about 20%, by weight of a disclosed UNC13A oligonucleotide or a pharmaceutically acceptable salt thereof. Such a tablet may include for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of a disclosed UNC13A oligonucleotide, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g., about 2% to about 4% sodium starch glycolate by weight.

In another contemplated embodiment, a pharmaceutical tablet formulation for oral administration of a disclosed UNC13A oligonucleotide comprises an intra-granular phase, wherein the intra-granular phase includes a disclosed UNC13A AON or a pharmaceutically acceptable salt thereof (such as a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 20% by weight of the tablet. For example, a pharmaceutically acceptable tablet for oral use may comprise about 0.5% to 10% by weight of a disclosed UNC13A AON, e.g., a disclosed UNC13A AON or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In another example, a pharmaceutically acceptable tablet for oral use may comprise an intra-granular phase, comprising about 5 to about 10% by weight of a disclosed UNC13A AON, e.g., a disclosed UNC13A AON or a pharmaceutically acceptable salt thereof, about 40% by weight mannitol, about 8% by weight microcrystalline cellulose, about 5% by weight hydroxypropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments the pharmaceutical composition may contain an enteric coating comprising about 13% or about 15%, 16%, 17% or 18% by weight, e.g., AcyrlEZE® (see, e.g., PCT Publication No. WO 2010/054826, which is hereby incorporated by reference in its entirety).

The rate at which the coating dissolves and the active ingredient is released is its dissolution rate. In an embodiment, a contemplated tablet may have a dissolution profile, e.g., when tested in a USP/EP Type 2 apparatus (paddle) at 100 rpm and 37° C. in a phosphate buffer with a pH of 7.2, of about 50% to about 100% of the UNC13A oligonucleotide releasing after about 120 minutes to about 240 minutes, for example after 180 minutes. In another embodiment, a contemplated tablet may have a dissolution profile, e.g., when tested in a USP/EP Type 2 apparatus (paddle) at 100 rpm and 37° C. in diluted HCl with a pH of 1.0, where substantially none of the UNC13A oligonucleotide is released after 120 minutes. A contemplated tablet, in another embodiment, may have a dissolution profile, e.g., when tested in USP/EP Type 2 apparatus (paddle) at 100 rpm and 37° C. in a phosphate buffer with a pH of 6.6, of about 10% to about 30%, or not more than about 50% of the UNC13A oligonucleotide releasing after 30 minutes.

In some embodiments, methods provided herein may further include administering at least one other agent that is directed to treatment of diseases and disorders disclosed herein. In one embodiment, contemplated other agents may be co-administered (e.g., sequentially or simultaneously).

### Dosage and Frequency of Administration

The dosage or amounts described below refer either to the oligonucleotide or a pharmaceutically acceptable salt thereof.

In some embodiments, methods described herein include administering at least 1 μg, at least 5 μg, at least 10 μg, at least 20 μg, at least 30 μg, at least 40 μg, at least 50 μg, at least 60 μg, at least 70 μg, at least 80 μg, at least 90 μg, or at least 100 μg of a UNC13A antisense oligonucleotide e.g., a UNC13A oligonucleotide. In some embodiments, methods include administering from 10 mg to 500 mg, from 1 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of a UNC13A antisense oligonucleotide.

In some embodiments, methods described herein include administering formulations that include about 10 mg, 15 mg, 20 mg, 25 mg, 30 mg, 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 600 mg, 700 mg, 800 mg, 900 mg, 1 g, 1.5 g, 2.0 g, 2.5 g, 3.0 g, 3.5 g, 4.0 g, 4.5 g, or 5.0 g of a disclosed UNC13A oligonucleotide. In some embodiments, a formulation may include about 40 mg, 80 mg, or 160 mg of a disclosed UNC13A oligonucleotide. In some embodiments, a formulation may include at least 100 μg of a disclosed UNC13A oligonucleotide. For example, formulations may include about 0.1 mg, 0.2 mg, 0.3 mg, 0.4 mg, 0.5 mg, 1 mg, 5 mg, 10 mg, 15 mg, 20 mg, 25 mg, or 30 mg of a disclosed UNC13A oligonucleotide. The amount administered will depend on variables such as the type and extent of disease or indication to be treated, the overall health and size of the patient, the in vivo potency of the UNC13A oligonucleotide, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study. Dosing frequency can vary, depending on factors such as route of administration, dosage amount and the disease being treated. Exemplary dosing frequencies are once per day, once per week and once every two weeks. In some embodiments, dosing is once per day for 7 days. In some embodiments, dosing is once every 4 weeks, once every 5 weeks, once every 6 weeks, once every 7 weeks, once every 8 weeks, once every 9 weeks, once every 10 weeks, once every 11 weeks, or once every 12 weeks. In some embodiments, dosing is once a month to every three months. In some embodiments, dosing is once every 2 weeks for three dose, then monthly, bimonthly, or every three or four months.

### Combination Therapies

In various embodiments, a UNC13A AON as disclosed herein can be administered in combination with one or more additional therapies. The combination therapy of the disclosed oligonucleotide and the one or more additional therapies can, in some embodiments, be synergistic in treating any of amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), TDP43 proteinopathies (e.g., chronic traumatic encephalopathy, Perry Syndrome, Dementia with Lewy body in association with Parkinson's disease, Parkinson's disease with or without dementia, and Limbic-predominant age-related TDP-43 encephalopathy (LATE)), epilepsy, Cerebral Age-Related TDP-43 With Sclerosis (CARTS), facial onset sensory and motor neuronopathy, Guam Parkinson-dementia complex, multisystem proteinopathy, CTE, and synaptic diseases like autism.

Non-limiting examples of therapies for Parkinson's disease (PD) include: deep brain stimulation, levodopa and carbidopa (duopa, rytary, Sinemet, inbrija), istradefylline (nourianz), safinamide (xadago), pramipexole (Mirapex), rotigotine (neupro), ropinirole (requip), amantadine (gocovri, Symmetrel, osmolex), benztropine (Cogentin), trihexyphenidyl (artane), selegiline (eldepryl, zelapar), rasagiline, entacapone (comtan), opicapone (ongentys), tolcapone (tasmar), apomorphine (apokyn, kynmobi), exenatide, lingzhi, BIIB054, BIIB094, Caffeine, sarizotan, Nuplazid, and embryonic dopamine cell implantation.

Non-limiting examples of therapies for Alzheimer's disease (AD) include aducanamab (Aduhlem), memantine (Namenda), Donepezil (Aricept), Rivastigmine (Exelon), Galantamine (razadyne), Namzeric, Suvorexant (belsomra), and lecanemab.

A non-limiting example of a therapy for amyotrophic lateral sclerosis (ALS) is pridopidine.

Non-limiting examples of therapies for frontotemporal dementia (FTD) include olanzapine (Zyprexa), quetiapine (Seroquel), SSRIs (citalopram (Cipramil), dapoxetine (Priligy), escitalopram (Cipralex), fluoxetine (Prozac or Oxactin), fluvoxamine (Faverin), paroxetine (Seroxat), sertraline (Lustral), vortioxetine (Brintellix)), divalproex sodium (Depakote), carbamazepine (Tegretol), and medroxyprogestrone.

Non-limiting examples of therapies for epilepsy include Brivaracetam (briviact), cannabidiol (epidiolex), carbamazepine (carbatrol, Tegretol), cenobamate (xcopri), diazepam (valium), lorazepam (Ativan), clonazepam (klonopin), eslicarbazepine (aptiom), ethosuximide (zarontin), felbamate (felbatol), fenfluramine (fintepla), lacosamide (VIMPAT), lamotrigine (Lamictal), levetiracetam (Keppra), oxcarbazepine (oxtellar xr, Trileptal), perampanel (fycompa), phenobarbital, phenytoin (dilantin), pregabalin (lyrica), tiagabine (gabitril), topiramate (topamax), valproate (depakene, depakote), and zonisamide (zonegran).

Example additional therapies include any of Riluzole (Rilutek), PrimeC, Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), pridopidine, dual AON intrathecal administration (e.g., BIIB067, BIIB078, and BIIB105), BIIB100, levodopa/carbidopa, dopaminergic agents (e.g., ropinirole, pramipexole, rotigotine), medroxyprosterone, KCNQ2/KCNQ3 openers (e.g., retigabine, XEN1101, or QRL-101), bioactive scaffolds, anticonvulsants and psychostimulant agents. Additional therapies can further include breathing care, physical therapy, occupational therapy, speech therapy, and nutritional support. Further non-limiting examples of additional therapies include any of deep brain stimulation, levodopa and carbidopa (duopa, rytary, Sinemet, inbrija), istradefylline (nourianz), safinamide (xadago), pramipexole (Mirapex), rotigotine (neupro), ropinirole (requip), amantadine (gocovri, Symmetrel, osmolex), benztropine (Cogentin), trihexyphenidyl (artane), selegiline (eldepryl, zelapar), rasagiline, entacapone (comtan), opicapone (ongentys), tolcapone (tasmar), apomorphine (apokyn, kynmobi), exenatide, lingzhi, BIIB054, BIIB094, Caffeine, sarizotan, embryonic dopamine cell implantation, aducanamab (Aduhlem), memantine (Namenda), Donepezil (Aricept), Rivastigmine (Exelon), Galantamine (razadyne), Namzeric, Suvorexant (belsomra), lecanemab, olanzapine (Zyprexa), quetiapine (Seroquel), SSRIs (citalopram (Cipramil), dapoxetine (Priligy), escitalopram (Cipralex), fluoxetine (Prozac or Oxactin), fluvoxamine (Faverin), paroxetine (Seroxat), sertraline (Lustral), vortioxetine (Brintellix))), divalproex sodium (Depakote), carbamazepine (Tegretol), medroxyprogestrone, Brivaracetam (briviact), cannabidiol (epidiolex), carbamazepine (carbatrol, Tegretol), cenobamate (xcopri), diazepam (valium), lorazepam (Ativan), clonazepam (klonopin), eslicarbazepine (aptiom), ethosuximide (zarontin), felbamate (felbatol), fenfluramine (fintepla), lacosamide (VIMPAT), lamotrigine (Lamictal), levetiracetam (Keppra), oxcarbazepine (oxtellar xr, Trileptal), perampanel (fycompa), phenobarbital, phenytoin (dilantin), pregabalin (lyrica), tiagabine (gabitril), topiramate (topamax), valproate (depakene, depakote), and zonisamide (zonegran). In various embodiments, an additional therapy can be a second antisense oligonucleotide. As an example, the second antisense oligonucleotide may target a UNC13A transcript (e.g., UNC13A pre-mRNA, mature UNC13A mRNA) to modulate the expression levels of full length UNC13A protein.

Non-limiting examples of therapies for spinal cord injury includes bioactive scaffolds, such as bioactive scaffolds with enhanced supramolecular motion. Further details of example bioactive scaffolds as therapies for spinal cord injury is described in Alvarez et al., “Bioactive scaffolds with enhanced supramolecular motion promote recovery from spinal cord injury.” Science, 374, 848-856 (2021), which is hereby incorporated by reference in its entirety.

In various embodiments, the disclosed oligonucleotide and the one or more additional therapies can be conjugated to one another and provided in a conjugated form. Further description regarding conjugates involving the disclosed oligonucleotide is described below. In various embodiments, the disclosed oligonucleotide and one or more additional therapies are provided concurrently. In various embodiments, the disclosed oligonucleotide and one or more additional therapies are provided simultaneously. In various embodiments, the disclosed oligonucleotide and one or more additional therapies are provided sequentially.

### Conjugates

In certain embodiments, provided herein are oligomeric compounds, which comprise an oligonucleotide (e.g., UNC13A oligonucleotide) and optionally one or more conjugate groups and/or terminal groups. Conjugate groups include one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### Conjugate Groups

In certain embodiments, a UNC13A AON is covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge, and clearance. In particular embodiments, conjugate groups modify the circulation time (e.g., increase) of the oligonucleotides in the bloodstream such that increased concentrations of the oligonucleotides are delivered to the brain. In particular embodiments, conjugate groups modify the residence time (e.g., increase residence time) of the oligonucleotides in a target organ (e.g., brain) such that increased residence time of the oligonucleotides improves their performance (e.g., efficacy). In particular embodiments, conjugate groups increase the delivery of the oligonucleotide to the brain through the blood brain barrier and/or brain parenchyma (e.g., through receptor mediated transcytosis). In particular embodiments, conjugate groups enable the oligonucleotide to target a specific organ (e.g., the brain). In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. NY. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-0-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO20141/179620).

### Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, dyes, bile acids, and phenylbutyric acid. In particular embodiments, conjugate moieties are selected from a peptide, a lipid, N-acetylgalactosamine (GalNAc), cholesterol, vitamin E, lipoic acid, panthothenic acid, polyethylene glycol, an antibody (e.g., an antibody for crossing the blood brain barrier such as anti-transferrin receptor antibody), or a cell-penetrating peptide (e.g., transactivator of transcription (TAT) and penetratin).

In certain embodiments, a conjugate moiety comprises an active drug substance, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indomethacin, a barbiturate, a cephalosporin, a sulfa drug, an antidiabetic, an antibacterial or an antibiotic.

### Conjugate Linkers

Conjugate moieties are attached to a UNC13A AON through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbon chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise 3 linker-nucleosides.

In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid.

In certain embodiments, it is desirable for a conjugate group to be cleaved from the UNC13A AON. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxy adenosine.

### Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside. In various embodiments, terminal groups comprise one or more spacers.

### Diagnostic Methods

The disclosure also provides a method of diagnosing a patient with a neurological disease that relies upon detecting levels of UNC13A expression signal in one or more biological samples of a patient. As used herein, the term “UNC13A expression signal” can refer to any indication of UNC13A gene expression, or gene or gene product activity. UNC13A gene products include RNA (e.g., mRNA), peptides, and proteins. Indices of UNC13A gene expression that can be assessed include, but are not limited to, UNC13A gene or chromatin state, UNC13A gene interaction with cellular components that regulate gene expression, UNC13A gene product expression levels (e.g., expression levels of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to any one of SEQ ID NO: 5057-5065 or SEQ ID NOs: 5206-5208, or interaction of UNC13A RNA or protein with transcriptional, translational, or post-translational processing machinery.

Detection of UNC13A expression signal may be accomplished through in vivo, in vitro, or ex vivo methods. In a preferred embodiment, methods of the disclosure may be carried out in vitro. Methods of detecting may involve detection in blood, serum, fecal matter, tissue, cerebrospinal fluid, spinal fluid, urine, extracellular vesicles (for example, CSF exosomes), or cells of a patient. Detection may be achieved by measuring expression signal of UNC13A transcripts (for example, a UNC13A pre-mRNA) comprising a sequence that shares at least 90% (e.g., 90%, 91%, 92%, 93%, 94%, 95%, 96%, 97%, 98%, 99%, or 100%) identity to SEQ ID NO: 5057-5065 or SEQ ID NOs: 5206-5208 in whole tissue, tissue explants, cell cultures, dissociated cells, cell extract, extracellular vesicles (for example, CSF exosomes), or body fluids, including blood, spinal fluid, cerebrospinal fluid, urine, lymphatic fluid, plasma, or serum. Methods of detection include assays that measure levels of UNC13A gene product expression such as Western blotting, FACS, ELISA, other quantitative binding assays, cell or tissue growth assays, Northern blots, quantitative or semi-quantitative polymerase chain reaction, medical imaging methods (e.g., MRI), or immunostaining methods (e.g., immunohistochemistry or immunocytochemistry).

### Modifications in General

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine(methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or R such as for sugar anomers, or as (D) or (L), such as for amino acids, etc. Compounds provided herein that are drawn or described as having certain stereoisomeric configurations include only the indicated compounds. Compounds provided herein that contain stereocenters that are drawn or described with undefined stereochemistry included all such possible isomers, including their stereorandom and optically pure forms, unless specified otherwise. Likewise, all tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, 170 or 180 in place of 160, and ³³S, ³⁴S, ³⁵S, or ³⁶S in place of 32S. In certain embodiments, non-radioactive isotopic substitutions may impart new properties on the oligomeric compound that are beneficial for use as a therapeutic or research tool.

### EXAMPLES

The disclosure is further illustrated by the following examples. The examples are provided for illustrative purposes only, and are not to be construed as limiting the scope or content of the disclosure in any way.

### Example 1: Design and Selection of UNC13A Oligonucleotides

UNC13A AONs oligonucleotides that target a UNC13A transcript are designed and tested to identify UNC13A AONs capable of reducing quantity of UNC13A transcripts (e.g., mis-spliced UNC13A transcripts). Such UNC13A AONs include UNC13A parent oligonucleotides represented by any of SEQ ID NOs: 1-1264 or UNC13A oligonucleotide variants represented by SEQ ID NOs: 2529-3792. The UNC13A parent oligonucleotides are 25 nucleosides in length. Each of the nucleosides of the UNC13A parent oligonucleotides are modified nucleosides with 2′MOE sugar moieties, and each “C” is replaced with a 5-MeC. Additionally, each of the internucleoside linkages between the nucleosides of the UNC13A oligonucleotides are phosphorothioate internucleoside linkages.

Generally, the length of the UNC13A antisense oligonucleotides are 25 oligonucleotide units in length. However, variants of the UNC13A antisense oligonucleotides were also designed with varying lengths (e.g., 23mers, 21mers, or 19mers). Examples of these variant UNC13A antisense oligonucleotides were designed to include a subset of the sequences of SEQ ID NOs: 2529-3792.

TABLE 6A
Example UNC13A AONs (including UNC13A
oligonucleotides with two spacers)

| | Oligonucleotide Sequence | |
|---|---|---|
| SEQ | (where S indicates | |
| ID | presence of a Spacer)* | |
| No: | (5′ → 3′) | Length |
| 5133 | GGGCAGSCAGGAATGGTGSGTGGAA | 25 |
| 5134 | AGGGAGSAGTTTTCCAGGSAAAGGG | 25 |
| 5135 | GCAGCTSGAAGAGACATASCCAGAC | 25 |
| 5136 | TCCACCSGCCACTCACAASCATCCA | 25 |
| 5137 | GAAAGTSTCATGGAGAGTSCAAGGG | 25 |
| 5138 | GGGCAGGCSGGAATGGTGSGTGGAA | 25 |
| 5139 | AGGGAGGASTTTTCCAGGSAAAGGG | 25 |
| 5140 | GCAGCTGGSAGAGACATASCCAGAC | 25 |
| 5141 | TCCACCAGSCACTCACAASCATCCA | 25 |
| 5142 | GAAAGTGTSATGGAGAGTSCAAGGG | 25 |
| 5143 | GCAGGCASGAATGGTSAGTGGAA | 23 |
| 5144 | GGGCAGGSAGGAATGSTGAGTGG | 23 |
| 5145 | GGCAGGCSGGAATGGSGAGTGGA | 23 |
| 5146 | GTAAAGGSAATGGCASGGCGGGC | 23 |
| 5147 | AGGTAAASGGAATGGSATGGCGG | 23 |
| 5148 | GGTAAAGSGAATGGCSTGGCGGG | 23 |
| 5149 | AGGAGGTSACCCTGASTCTGGAC | 23 |
| 5150 | AGAGGAGSTGACCCTSAATCTGG | 23 |
| 5151 | GAGGAGGSGACCCTGSATCTGGA | 23 |
| 5152 | ACAAACGSCCCAATCSTGAGTGG | 23 |
| 5153 | ACACAAASGGCCCAASCCTGAGT | 23 |
| 5154 | CACAAACSGCCCAATSCTGAGTG | 23 |
| 5155 | TCTTTCCSGGAAACCSAGGCAGC | 23 |
| 5156 | GTTCTTTSCAGGAAASCCAGGCA | 23 |
| 5157 | TTCTTTCSAGGAAACSCAGGCAG | 23 |
| 5158 | TCCGCAASTTAATCCSCCTACCC | 23 |
| 5159 | GATCCGCSACTTAATSCACCTAC | 23 |
| 5160 | ATCCGCASCTTAATCSACCTACC | 23 |
| 5161 | CCCACCCSTCTAACTSCCCCAAA | 23 |
| 5162 | CACCCACSCATCTAASTACCCCA | 23 |
| 5163 | ACCCACCSATCTAACSACCCCAA | 23 |
| 5164 | AAGTGTCSTGGAGAGSGCAAGGG | 23 |
| 5165 | GAAAGTGSCATGGAGSGTGCAAG | 23 |
| 5166 | AAAGTGTSATGGAGASTGCAAGG | 23 |

* Unless otherwise noted, each of the nucleosides of antisense oligonucleotides shown are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer as indicated by S is not a nucleoside. S represents a spacer of Formula (Iia′) as disclosed herein.

TABLE 6B
Example UNC13A AONs (including UNC13A
oligonucleotides with two spacers)

| | Oligonucleotide Sequence | |
|---|---|---|
| SEQ | (where S indicates | |
| ID | presence of a Spacer)* | |
| No: | (5′ → 3′) | Length |
| 5168 | AGTTCASCCACACATCCTSCCATTC | 25 |
| 5169 | AGTTCAACSACACATCCTSCCATTC | 25 |
| 5170 | GTTCAACSACACATCSTTCCATT | 23 |
| 5171 | AGTTCAASCACACATSCTTCCAT | 23 |
| 5172 | TTCAACCSCACATCCSTCCATTC | 23 |
| 5173 | CTTGCASTAGTTCAACCASACATCC | 25 |
| 5174 | CTTGCAATSGTTCAACCASACATCC | 25 |
| 5175 | TTGCAATSGTTCAACSACACATC | 23 |
| 5176 | CTTGCAASAGTTCAASCACACAT | 23 |
| 5177 | TGCAATASTTCAACCSCACATCC | 23 |
| 5178 | ACACATSCATCCATCCATSCATTCA | 25 |
| 5179 | ACACATCCSTCCATCCATSCATTCA | 25 |
| 5180 | CACATCCSTCCATCCSTCCATTC | 23 |
| 5181 | ACACATCSATCCATCSATCCATT | 23 |
| 5182 | ACATCCASCCATCCASCCATTCA | 23 |
| 5183 | CAGTAASTCAACCACACASCCATCC | 25 |
| 5184 | CAGTAATTSAACCACACASCCATCC | 25 |
| 5185 | AGTAATTSAACCACASATCCATC | 23 |
| 5186 | CAGTAATSCAACCACSCATCCAT | 23 |
| 5187 | GTAATTCSACCACACSTCCATCC | 23 |
| 5188 | ACCTTTSAGTAATTCAACSACACAT | 25 |
| 5189 | ACCTTTCASTAATTCAACSACACAT | 25 |
| 5190 | CCTTTCASTAATTCASCCACACA | 23 |
| 5191 | ACCTTTCSGTAATTCSACCACAC | 23 |
| 5192 | CTTTCAGSAATTCAASCACACAT | 23 |
| 5193 | CTCTTTSATCCATCCACASACCCAC | 25 |
| 5194 | CTCTTTTASCCATCCACASACCCAC | 25 |
| 5195 | TCTTTTASCCATCCASACACCCA | 23 |
| 5196 | CTCTTTTSTCCATCCSCACACCC | 23 |
| 5197 | CTTTTATSCATCCACSCACCCAC | 23 |
| 5198 | ATCTACSCTTTTATCCATSCACACA | 25 |
| 5199 | ATCTACTCSTTTATCCATSCACACA | 25 |
| 5200 | TCTACTCSTTTATCCSTCCACAC | 23 |
| 5201 | ATCTACTSTTTTATCSATCCACA | 23 |
| 5202 | CTACTCTSTTATCCASCCACACA | 23 |

* Unless otherwise noted, each of the nucleosides of antisense oligonucleotides shown are modified nucleosides with 2′-O-(2-methoxyethyl) (2′-MOE) sugar moieties, each “C” is replaced with a 5-methylcytosine (5-MeC), and all internucleoside linkages are phosphorothioate linkages. A spacer as indicated by S is not a nucleoside. S represents a spacer of Formula (Iia′) as disclosed herein.

### Example 2: Methods for Evaluating UNC13A Antisense Oligonucleotides

UNC13A antisense oligonucleotides were evaluated in iPSC derived human motor neurons (hMN). The cells were seeded in 96-well plates at a density of 50,000 cells/well. Antisense oligonucleotide (AON) to TDP43 was transfected with Endoporter (Gene Tools, Philomath, OR, USA) to decrease expression of the full length UNC13a transcript. Vehicle control consisted of motor neuron treatment with Endoporter alone. Positive controls included cells that were treated with TDP43 AON alone (“AON TDP43” or “TDP43 AON”).

TDP43 AON is a gapmer oligonucleotide and has the following sequence and chemistry: 5′ A*A*G*G*C*T*T*C*A*T*A*T*T*G*T*A*C*T*T*T 3′ (SEQ ID NO: 5167) where *=phosphorothioate, underlined=DNA, other=2′MOE RNA; each “C” is 5-MeC.

To evaluate UNC13a AON ability to restore full length UNC13A (UNC13A FL) mRNA (also referred to as correctly spliced UNC13A (UNC13A CS) mRNA), antisense oligonucleotides to UNC13A were co-incubated with TDP43 AON in Endoporter in media before addition to the cells. After 72 hours, antisense oligonucleotides and Endoporter were washed out and replaced with fresh media alone. After 72 additional hours, RNA was collected from the 96-well plates for RT-qPCR. RNA was isolated, cDNA generated and multiplexed RT-qPCR assay performed with Taqman probes for UNC13A full length transcript and reference GAPDH quantification.

Transcript levels (e.g., UNC13A full length transcript or TDP43 transcript) were detected by RT-qPCR using Taqman. Specifically, RT-qPCR was performed for detecting GAPDH using Thermofisher® TaqMan Gene Expression Assay Hs03929097_g1. RT-qPCR was performed for detecting UNC13A-FL transcripts using Thermofisher® TaqMan Gene Expression Assay Hs00392638_ml.

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

UNC13A-FL (Ct) was normalized to GAPDH (deltaCt). To visualize the quantitative changes (e.g., % decrease of UNC13A-FL), the normalized UNC13A-FL signal was further normalized to the vehicle (treated with Endoporter alone, deltadeltaCt). Relative quantity (RQ) of transcript level was calculated using the equation RQ=2^{-deltadeltaCt} and is used to describe the treatment condition comparison to normal, healthy levels (1.0). RQ values for UNC13A FL were normalized using the following formula:

$$( ( ( RQAON - RQTDP 43 ) / ( R Qendo - RQTDP 43 ) ) * 100$$

Table 7 shows the RT-qPCR results of UNC13A AONs with spacers and performance of UNC13A AONs without spacers in human motor neurons. UNC13A AONs (e.g., UNC13A oligonucleotides without spacers or with one or two spacers were tested for their ability to increase or restore full-length UNC13A mRNA (i.e., mRNA from which full-length UNC13A protein is translated) levels. In some cases, UNC13A AONs with spacers increased full-length UNC13A mRNA (“UNC13A FL”), also referred to herein as correctly spliced UNC13A (UNC13A CS). In some cases, UNC13A AONs without spacers increased full-length UNC13A mRNA or correctly spliced UNC13A mRNA (UNC13A CS)). Specific AON sequences are labeled according to their corresponding SEQ ID NO.

As shown in Table 7, a 500 nM dose of SEQ ID NO: 5153 (ACACAAASGGCCCAASCCTGAGT with two spacers) rescued full length UNC13A mRNA to 55.69%. Comparatively, a 500 nM dose of SEQ ID NO: 2788 (ACACAAACGGCCCAATCCTGAGT with no spacers) rescued full length UNC13A mRNA to 41.87%. This indicates that the addition of spacers of SEQ ID NO: 5153 improves the performance in comparison to the UNC13A AON counterpart without spacers (e.g., SEQ ID NO: 2788).

Additionally shown in Table 7, a 200 nM dose of SEQ ID NO: 5159 (GATCCGCSACTTAATSCACCTAC with two spacers) rescued full length UNC13A mRNA to 55.88%. A 500 nM dose of SEQ ID NO: 5159 (GATCCGCSACTTAATSCACCTAC with two spacers) rescued full length UNC13A mRNA to 66.62%. Comparatively, a 200 nM dose of SEQ ID NO: 3547 (GATCCGCAACTTAATCCACCTAC with no spacers) rescued full length UNC13A mRNA to 43.88%. A 500 nM dose of SEQ ID NO: 3547 (GATCCGCAACTTAATCCACCTAC with no spacers) rescued full length UNC13A mRNA to 66.98%. This indicates that at 200 nM and 500 nM doses, the performance of an AON with SEQ ID NO: 5159 (with spacers) is similar to the performance of the UNC13A AON counterpart without spacers (e.g., SEQ ID NO: 3547).

Additionally shown in Table 7, a 200 nM dose of SEQ ID NO: 5162 (CACCCACSCATCTAASTACCCCA with two spacers) rescued full length UNC13A mRNA to 47.94%. A 500 nM dose of SEQ ID NO: 5162 (CACCCACSCATCTAASTACCCCA with two spacers) rescued full length UNC13A mRNA to 83.19%. Comparatively, a 200 nM dose of SEQ ID NO: 3652 (CACCCACCCATCTAACTACCCCA with no spacers) rescued full length UNC13A mRNA to 47.68%. A 500 nM dose of SEQ ID NO: 3652 (CACCCACCCATCTAACTACCCCA with no spacers) rescued full length UNC13A mRNA to 41.16%. This indicates that at a 200 nM dose, the performance of SEQ ID NO: 5162 (with spacers) is similar to the performance of the UNC13A AON counterpart without spacers (e.g., SEQ ID NO: 3652). However, at a 500 nM dose, the addition of spacers of SEQ ID NO: 5162 improves the performance in comparison to the UNC13A AON counterpart without spacers (e.g., SEQ ID NO: 3652).

Additionally shown in Table 7, a 500 nM dose of SEQ ID NO: 5161 (CCCACCCSTCTAACTSCCCCAAA with two spacers) rescued full length UNC13A mRNA to 43.10%. Comparatively, a 500 nM dose of SEQ ID NO: 3650 (CCCACCCATCTAACTACCCCAAA with no spacers) rescued full length UNC13A mRNA to 23.37%. This indicates that at a 500 nM dose, the addition of spacers improves the performance of an AON with SEQ ID NO: 5161 in comparison to the UNC13A AON counterpart without spacers (e.g., SEQ ID NO: 3650).

Additionally shown in Table 7, a 200 nM dose of SEQ ID NO: 5164 (AAGTGTCSTGGAGAGSGCAAGGG with two spacers) rescued full length UNC13A mRNA to 35.39%. A 500 nM dose of SEQ ID NO: 5164 (AAGTGTCSTGGAGAGSGCAAGGG with two spacers) rescued full length UNC13A mRNA to 6.60%. Comparatively, a 200 nM dose of SEQ ID NO: 3774 (AAGTGTCATGGAGAGTGCAAGGG with no spacers) rescued full length UNC13A mRNA to 19.04%. A 500 nM dose of SEQ ID NO: 3774 (AAGTGTCATGGAGAGTGCAAGGG with no spacers) rescued full length UNC13A mRNA to −34.58%. This indicates that at 200 nM and 500 nM doses, the addition of spacers improves the performance of an AON with SEQ ID NO: 5164 in comparison to the UNC13A AON counterpart without spacers (e.g., SEQ ID NO: 3774).

Additionally shown in Table 7, a 200 nM dose of SEQ ID NO: 5142 (GAAAGTGTSATGGAGAGTSCAAGGG with two spacers) rescued full length UNC13A mRNA to 67.77%. A 500 nM dose of SEQ ID NO: 5142 (GAAAGTGTSATGGAGAGTSCAAGGG with two spacers) rescued full length UNC13A mRNA to 84.79%. Comparatively, a 200 nM dose of SEQ ID NO: 1248 (GAAAGTGTCATGGAGAGTGCAAGGG with no spacers) rescued full length UNC13A mRNA to 22.48%. A 500 nM dose of SEQ ID NO: 1248 (GAAAGTGTCATGGAGAGTGCAAGGG with no spacers) rescued full length UNC13A mRNA to 24.84%. This indicates that at 200 nM and 500 nM doses, the addition of spacers improves the performance of an AON with SEQ ID NO: 5142 in comparison to the UNC13A AON counterpart without spacers (e.g., SEQ ID NO: 1248).

Altogether, these results demonstrate that different UNC13A AONs including two spacers are capable of increasing UNC13A-FL mRNA to levels that are comparable to or improved beyond their non-spacer counterparts.

TABLE 7
Performance of UNC13A AONs in UNC13A full length assay
(UNC13A oligonucleotides without spacers, or with one or two spacers).

| | Oligonucleotide | QPCR potency | | QPCR potency | |
|---|---|---|---|---|---|
| | Sequence* (where S | 200 nM | 200 nM | 500 nM | 500 nM |
| SEQ ID | indicates presence | Normalized | Normalized | Normalized | Normalized |
| NO: | of a Spacer) (5′ → 3′) | CS | CS SD | CS | CS SD |
| 42 | GAAGAGCTGGGCAGGCAGGAATGGT | 3.79367 | 0.480565 | −63.053 | 0.19433 |
| 66 | TGTGGATGGTGTGGCCAGAAAGAGG | 60.6986 | 0.724229 | −23.435 | 0.36397 |
| 77 | GGCCAGGAGAGTGTGGATGGTGTGG | 154.828 | 1.127288 | 434.631 | 2.32539 |
| 101 | ATGGCATGGCGGGCAGTCTCAGAGG | 37.1601 | 0.448286 | 12.6634 | 0.41631 |
| 108 | AAAGGGAATGGCATGGCGGGCAGTC | −26.139 | 0.13718 | 22.9464 | 0.49028 |
| 112 | AGGTAAAGGGAATGGCATGGCGGGC | −5.3242 | 0.383369 | −37.199 | 0.17903 |
| 2639 | GGTAAAGGGAATGGCATGGCGGG | −29.228 | 0.227001 | −33.566 | 0.20089 |
| 5148 | GGTAAAGSGAATGGCSTGGCGGG | −16.331 | 0.304608 | −29.739 | 0.22392 |
| 2640 | AGGTAAAGGGAATGGCATGGCGG | −13.385 | 0.322337 | −40.436 | 0.15956 |
| 5147 | AGGTAAASGGAATGGSATGGCGG | −13.664 | 0.320657 | −24.027 | 0.2583 |
| 2638 | GTAAAGGGAATGGCATGGCGGGC | −29.989 | 0.222419 | −26.677 | 0.24235 |
| 5146 | GTAAAGGSAATGGCASGGCGGGC | 25.771 | 0.557959 | −18.428 | 0.29199 |
| 115 | TCCAGGTAAAGGGAATGGCATGGCG | −7.8349 | 0.268854 | 40.2877 | 0.61503 |
| 127 | AGGGAGGAGTTTTCCAGGTAAAGGG | −32.255 | 0.392785 | −25.109 | 0.4256 |
| 5134 | AGGGAGSAGTTTTCCAGGSAAAGGG | 6.97605 | 0.572906 | −3.6889 | 0.52394 |
| 5139 | AGGGAGGASTTTTCCAGGSAAAGGG | −70.51 | 0.217148 | −78.696 | 0.17957 |
| 162 | AGAGGAGGTGACCCTGAATCTGGAC | 129.033 | 0.93262 | 110.34 | 1.0505 |
| 2689 | GAGGAGGTGACCCTGAATCTGGA | 169.855 | 1.024819 | 164.814 | 1.30762 |
| 5151 | GAGGAGGSGACCCTGSATCTGGA | −72.457 | 0.658323 | 138.629 | 1.28191 |
| 2690 | AGAGGAGGTGACCCTGAATCTGG | 171.498 | 1.135638 | 3.26987 | 0.54512 |
| 5150 | AGAGGAGSTGACCCTSAATCTGG | 7.49253 | 0.565048 | −57.946 | 0.25617 |
| 2688 | AGGAGGTGACCCTGAATCTGGAC | 44.7095 | 0.740715 | 125.001 | 1.1197 |
| 5149 | AGGAGGTSACCCTGASTCTGGAC | −44.26 | 0.320771 | 12.8179 | 0.59018 |
| 195 | GAGAGTCAACCTGGAGGCCAAGGTG | 32.5688 | 0.559505 | 57.0338 | 0.7355 |
| 200 | GTAGTGAGAGTCAACCTGGAGGCCA | 70.681 | 0.905428 | 67.6819 | 0.88671 |
| 203 | TGAGTAGTGAGAGTCAACCTGGAGG | 91.2119 | 1.033573 | 59.7857 | 0.83742 |
| 244 | CCTGAGTGGTTAGGGCTGGAATAGA | 46.7792 | 0.756244 | −24.332 | 0.3124 |
| 248 | CAATCCTGAGTGGTTAGGGCTGGAA | −8.5 | 0.411217 | 73.8646 | 0.9253 |
| 254 | ACGGCCCAATCCTGAGTGGTTAGGG | 66.8521 | 0.88153 | 45.3726 | 0.74746 |
| 260 | ACACAAACGGCCCAATCCTGAGTGG | −11.42 | 0.462541 | 75.8487 | 0.8839 |
| 2787 | CACAAACGGCCCAATCCTGAGTG | −11.128 | 0.463949 | −41.939 | 0.31518 |
| 5154 | CACAAACSGCCCAATSCTGAGTG | 50.7872 | 0.762897 | −14.314 | 0.44857 |
| 2788 | ACACAAACGGCCCAATCCTGAGT | −27.43 | 0.385239 | 41.8694 | 0.71984 |
| 5153 | ACACAAASGGCCCAASCCTGAGT | −14.732 | 0.44655 | 55.6902 | 0.78657 |
| 2786 | ACAAACGGCCCAATCCTGAGTGG | −49.751 | 0.277462 | −20.306 | 0.41964 |
| 5152 | ACAAACGSCCCAATCSTGAGTGG | 105.349 | 1.026341 | 47.8744 | 0.74883 |
| 263 | CAGACACAAACGGCCCAATCCTGAG | −40.7843 | 0.3272 | −5.4793 | 0.5486 |
| 269 | CATACCCAGACACAAACGGCCCAAT | −21.2475 | 0.3317 | 14.4015 | 0.5542 |
| 273 | GAGACATACCCAGACACAAACGGCC | 47.2141 | 0.7590 | 116.3656 | 1.1906 |
| 283 | GCAGCTGGAAGAGACATACCCAGAC | 4.7146 | 0.5625 | −66.6389 | 0.2349 |
| 5135 | GCAGCTSGAAGAGACATASCCAGAC | 33.7840 | 0.6960 | −6.4957 | 0.5111 |
| 5140 | GCAGCTGGSAGAGACATASCCAGAC | 3.7461 | 0.5581 | 35.3538 | 0.7032 |
| 301 | TCTTTCCAGGAAACCCAGGCAGCTG | 17.5514 | 0.5395 | 30.5680 | 0.5952 |
| 303 | GTTCTTTCCAGGAAACCCAGGCAGC | 41.1141 | 0.5932 | −27.3254 | 0.2666 |
| 2830 | TTCTTTCCAGGAAACCCAGGCAG | −31.8396 | 0.2450 | −7.7732 | 0.3599 |
| 5157 | TTCTTTCSAGGAAACSCAGGCAG | −72.0800 | 0.0530 | −37.1813 | 0.2196 |
| 2831 | GTTCTTTCCAGGAAACCCAGGCA | −14.5643 | 0.3275 | 44.7209 | 0.6104 |
| 5156 | GTTCTTTSCAGGAAASCCAGGCA | −38.7597 | 0.2120 | −22.4863 | 0.2897 |
| 2829 | TCTTTCCAGGAAACCCAGGCAGC | 2.0002 | 0.4065 | −15.1244 | 0.3248 |
| 5155 | TCTTTCCSGGAAACCSAGGCAGC | −59.9899 | 0.1107 | −64.5153 | 0.0891 |
| 454 | CACGAATCTACCCACCAACTCATCC | −39.5824 | 0.2948 | −56.2924 | 0.2233 |
| 460 | TCTAGCCACGAATCTACCCACCAAC | 75.4170 | 0.7873 | −12.0685 | 0.4126 |
| 927 | GTCTGTCCACCAGCCACTCACAACC | 121.2337 | 0.9834 | −62.7895 | 0.1955 |
| 930 | TTCGTCTGTCCACCAGCCACTCACA | −18.2285 | 0.3863 | −45.7096 | 0.2686 |
| 937 | TCCATTTTTCGTCTGTCCACCAGCC | −63.8195 | 0.2479 | −54.2647 | 0.2917 |
| 1019 | GATCCGCAACTTAATCCACCTACCC | 88.0305 | 0.9091 | 44.9905 | 0.5436 |
| 3546 | ATCCGCAACTTAATCCACCTACC | 30.7027 | 0.4223 | 45.1113 | 0.5446 |
| 5160 | ATCCGCASCTTAATCSACCTACC | 38.8278 | 0.4913 | 50.8796 | 0.5936 |
| 3547 | GATCCGCAACTTAATCCACCTAC | 43.8789 | 0.5342 | 66.9823 | 0.7303 |
| 5159 | GATCCGCSACTTAATSCACCTAC | 55.8768 | 0.6360 | 66.6162 | 0.7272 |
| 3545 | TCCGCAACTTAATCCACCTACCC | 64.3790 | 0.7082 | 67.3066 | 0.7331 |
| 5158 | TCCGCAASTTAATCCSCCTACCC | 53.4236 | 0.6152 | 51.3988 | 0.5980 |
| 1124 | CACCCACCCATCTAACTACCCCAAA | 64.1844 | 0.6729 | 44.6783 | 0.4941 |
| 3651 | ACCCACCCATCTAACTACCCCAA | 41.2908 | 0.4631 | 64.1844 | 0.6729 |
| 5163 | ACCCACCSATCTAACSACCCCAA | 46.9810 | 0.4070 | 58.8250 | 0.6238 |
| 3652 | CACCCACCCATCTAACTACCCCA | 47.6810 | 0.5217 | 41.0564 | 0.4609 |
| 5162 | CACCCACSCATCTAASTACCCCA | 47.9432 | 0.5241 | 83.1895 | 0.8471 |
| 3650 | CCCACCCATCTAACTACCCCAAA | 72.0651 | 0.5775 | 23.3677 | 0.2988 |
| 5161 | CCCACCCSTCTAACTSCCCCAAA | 41.8445 | 0.4682 | 43.0965 | 0.4796 |
| 1248 | GAAAGTGTCATGGAGAGTGCAAGGG | 22.4754 | 0.5041 | 24.8358 | 0.5167 |
| 3775 | AAAGTGTCATGGAGAGTGCAAGG | 82.5919 | 0.8245 | 7.0300 | 0.4218 |
| 5166 | AAAGTGTSATGGAGASTGCAAGG | 61.0070 | 0.7095 | −4.8799 | 0.3583 |
| 3776 | GAAAGTGTCATGGAGAGTGCAAG | 55.7844 | 0.6816 | −19.7864 | 0.2789 |
| 5165 | GAAAGTGSCATGGAGSGTGCAAG | 40.0402 | 0.5977 | 3.6974 | 0.4040 |
| 3774 | AAGTGTCATGGAGAGTGCAAGGG | 19.0435 | 0.4858 | −34.5807 | 0.2001 |
| 5164 | AAGTGTCSTGGAGAGSGCAAGGG | 35.3880 | 0.5729 | 6.6020 | 0.4195 |
| 5137 | GAAAGTSTCATGGAGAGTSCAAGGG | −10.6183 | 0.3278 | 50.6900 | 0.6545 |
| 5142 | GAAAGTGTSATGGAGAGTSCAAGGG | 67.7691 | 0.7455 | 84.7852 | 0.8362 |
| 1260 | CACCATGGGAGAGAAAGTGTCATGG | 17.7544 | 0.4529 | 64.3335 | 0.7880 |

*Unless otherwise noted, each of the nucleosides of antisense oligonucleotides shown are modified nucleosides with 2′-O-(2-methoxyethyl) (2′-MO sugar moieties, each “C” is replaced with a 5-methylcytosine (5-MeC), and all internucleoside linkages are phosphorothioate linkages.
A spacer as indicated by S is not a nucleoside. S represents a spacer of Formula (Iia′) as disclosed herein.

### Example 3: Methods for Evaluating UNC13A Antisense Oligonucleotides

UNC13A antisense oligonucleotides were evaluated in iPSC derived human motor neurons (hMN). The cells were seeded in 96-well plates at a density of 40,000 cells/well. Antisense oligonucleotide (AON) to TDP43 was transfected with Endoporter (Gene Tools, Philomath, OR, USA) to decrease expression of the full length UNC13A transcript and increase expression of UNC13A cryptic exon. Vehicle control consisted of motor neuron treatment with Endoporter alone. Positive controls included cells that were treated with TDP43 AON alone (“AON TDP43” or “TDP43 AON”).

TDP43 AON is a gapmer oligonucleotide and has the following sequence and chemistry: 5′ A*A*G*G*C*T*T*C*A*T*A*T*T*G*T*A*C*T*T*T 3′ (SEQ ID NO: 5167) where *=phosphorothioate, underlined=DNA, other=2′MOE RNA; each “C” is 5-MeC.

To evaluate UNC13A AON ability to reduce UNC13A cryptic exon levels, antisense oligonucleotides to UNC13A were co-incubated with TDP43 AON in Endoporter in media before addition to the cells. After 72 hours, antisense oligonucleotides and Endoporter were washed out and replaced with fresh media alone. After six additional days, RNA was collected from the 96-well plates for RT-qPCR. RNA was isolated, cDNA generated and multiplexed RT-qPCR assay performed with Taqman probes for UNC13A cryptic exon, and reference GAPDH quantification.

Transcript levels (e.g., UNC13A cryptic exon, and TDP43 transcript) were detected by RT-qPCR using Taqman. Specifically, RT-qPCR was performed for detecting GAPDH using Thermofisher® TaqMan Gene Expression Assay Hs03929097_g1. UNC13a cryptic exon was detect using custom sequences.

### UNC13a Cryptic Exon:

Forward Primer:
(SEQ ID NO: 5203)
ATTGTTCTGCACGTCGGT
Reverse Primer:
(SEQ ID NO: 5204)
GTCTGGGTATGTCTCTTCCAG
Probe Sequence:
(SEQ ID NO: 5203)
AGTTCTTTCCAGGAAACCCAGGCA

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

UNC13A-cryptic (Ct) was normalized to GAPDH (deltaCt). To visualize the quantitative changes (e.g., % decrease of UNC13A-cryptic), the normalized UNC13A-cryptic signal was further normalized to the vehicle (treated with Endoporter alone, deltadeltaCt). Relative quantity (RQ) of transcript level was calculated using the equation RQ=2^{-deltadeltaCt} and is used to describe the treatment condition comparison to normal, healthy levels (1.0). RQ values for UNC13A cryptic were normalized using the following formula:

(((RQAON−RQendo)/(RQTDP43−RQendo))*100

Table 8 shows the RT-qPCR results of UNC13A AONs with spacers and performance of UNC13A AONs without spacers in human motor neurons.

As shown in Table 8, UNC13A AONs (e.g., UNC13A oligonucleotides without spacers or with one or two spacers) were tested for their ability to reduce UNC13A transcripts with a cryptic exon. In some cases, UNC13A AONs with spacers reduced UNC13A cryptic exon levels. In some cases, UNC13A AONs without spacers reduced UNC13A cryptic exon levels. Specific AON sequences are labeled according to their corresponding SEQ ID NO.

As shown in Table 8, a 200 nM dose of SEQ ID NO: 273 (GAGACATACCCAGACACAAACGGCC with no spacers) reduced UNC13A cryptic exon levels to 4.4%. A 50 nM dose of SEQ ID NO: 273 (GAGACATACCCAGACACAAACGGCC with no spacers) reduced UNC13A cryptic exon levels to 20.3%.

As shown in Table 8, a 200 nM dose of SEQ ID NO: 2831 (GTTCTTTCCAGGAAACCCAGGCA with no spacers) reduced UNC13A cryptic exon levels to 5.1%. A 50 nM dose of SEQ ID NO: 2831 (GTTCTTTCCAGGAAACCCAGGCA with no spacers) reduced UNC13A cryptic exon levels to 26.0%.

As shown in Table 8, a 200 nM dose of SEQ ID NO: 5156 (GTTCTTTSCAGGAAASCCAGGCA with two spacers) reduced UNC13A cryptic exon levels to 6.6%. A 50 nM dose of SEQ ID NO: 5156 (GTTCTTTSCAGGAAASCCAGGCA with two spacers) reduced UNC13A cryptic exon levels to 41.2%.

As shown in Table 8, a 200 nM dose of SEQ ID NO: 5140 (GCAGCTGGSAGAGACATASCCAGAC with two spacers) reduced UNC13A cryptic exon levels to 8.3%. A 50 nM dose of SEQ ID NO: 5140 (GCAGCTGGSAGAGACATASCCAGAC with two spacers) reduced UNC13A cryptic exon levels to 31.0%.

As shown in Table 8, a 200 nM dose of SEQ ID NO: 303 (GTTCTTTCCAGGAAACCCAGGCAGC with no spacers) reduced UNC13A cryptic exon levels to 8.8%. A 50 nM dose of SEQ ID NO: 303 (GTTCTTTCCAGGAAACCCAGGCAGC with no spacers) reduced UNC13A cryptic exon levels to 35.4%.

As shown in Table 8, a 200 nM dose of SEQ ID NO: 5135 (GCAGCTSGAAGAGACATASCCAGAC with two spacers) reduced UNC13A cryptic exon levels to 11.8%. A 50 nM dose of SEQ ID NO: 5135 (GCAGCTSGAAGAGACATASCCAGAC with two spacers) reduced UNC13A cryptic exon levels to 30.6%.

As shown in Table 8, a 200 nM dose of SEQ ID NO: 283 (GCAGCTGGAAGAGACATACCCAGACwith no spacers) reduced UNC13A cryptic exon levels to 13.8%. A 50 nM dose of SEQ ID NO: 283 (GCAGCTGGAAGAGACATACCCAGACwith no spacers) reduced UNC13A cryptic exon levels to 66.0%.

As shown in Table 8, a 200 nM dose of SEQ ID NO: 2830 (TTCTTTCCAGGAAACCCAGGCAG with no spacers) reduced UNC13A cryptic exon levels to 15.2%. A 50 nM dose of SEQ ID NO: 2830 (TTCTTTCCAGGAAACCCAGGCAG with no spacers) reduced UNC13A cryptic exon levels to 34.4%.

As shown in Table 8, a 200 nM dose of SEQ ID NO: 3585 (ACATCCATCCATCCATCCATTCA with no spacers) reduced UNC13A cryptic exon levels to 15.8%. A 50 nM dose of SEQ ID NO: 3585 (ACATCCATCCATCCATCCATTCA with no spacers) reduced UNC13A cryptic exon levels to 45.4%.

As shown in Table 8, a 200 nM dose of SEQ ID NO: 5155 (TCTTTCCSGGAAACCSAGGCAGC with two spacers) reduced UNC13A cryptic exon levels to 18.2%. A 50 nM dose of SEQ ID NO: 5155 (TCTTTCCSGGAAACCSAGGCAGC with two spacers) reduced UNC13A cryptic exon levels to 41.9%.

As shown in Table 8, a 200 nM dose of SEQ ID NO: 5157 (TTCTTTCSAGGAAACSCAGGCAG with two spacers) reduced UNC13A cryptic exon levels to 20.1%. A 50 nM dose of SEQ ID NO: 5157 (TTCTTTCSAGGAAACSCAGGCAG with two spacers) reduced UNC13A cryptic exon levels to 42.8%.

As shown in Table 8, a 200 nM dose of SEQ ID NO: 1059 (ACACATCCATCCATCCATCCATTCA with no spacers) reduced UNC13A cryptic exon levels to 23.1%. A 50 nM dose of SEQ ID NO: 1059 (ACACATCCATCCATCCATCCATTCA with no spacers) reduced UNC13A cryptic exon levels to 50.2%.

As shown in Table 8, a 200 nM dose of SEQ ID NO: 2829 (TCTTTCCAGGAAACCCAGGCAGC with no spacers) reduced UNC13A cryptic exon levels to 25%. A 50 nM dose of SEQ ID NO: 2829 (TCTTTCCAGGAAACCCAGGCAGC with no spacers) reduced UNC13A cryptic exon levels to 40.6%.

As shown in Table 8, a 200 nM dose of SEQ ID NO: 269 (CATACCCAGACACAAACGGCCCAAT with no spacers) reduced UNC13A cryptic exon levels to 27.2%. A 50 nM dose of SEQ ID NO: 269 (CATACCCAGACACAAACGGCCCAAT with no spacers) reduced UNC13A cryptic exon levels to 43.4%.

As shown in Table 8, a 200 nM dose of SEQ ID NO: 1142 (CTCTTTTATCCATCCACACACCCAC with no spacers) reduced UNC13A cryptic exon levels to 34.4%. A 50 nM dose of SEQ ID NO: 1142 (CTCTTTTATCCATCCACACACCCAC with no spacers) reduced UNC13A cryptic exon levels to 64.3%.

As shown in Table 8, a 200 nM dose of SEQ ID NO: 3670 (CTCTTTTATCCATCCACACACCC with no spacers) reduced UNC13A cryptic exon levels to 40.5%. A 50 nM dose of SEQ ID NO: 3670 (CTCTTTTATCCATCCACACACCC with no spacers) reduced UNC13A cryptic exon levels to 82.2%.

As shown in Table 8, a 200 nM dose of SEQ ID NO: 301 (TCTTTCCAGGAAACCCAGGCAGCTG with no spacers) reduced UNC13A cryptic exon levels to 41.6%. A 50 nM dose of SEQ ID NO: 301 (TCTTTCCAGGAAACCCAGGCAGCTG with no spacers) reduced UNC13A cryptic exon levels to 54.2%.

As shown in Table 8, a 200 nM dose of SEQ ID NO: 5165 (GAAAGTGSCATGGAGSGTGCAAG with two spacers) reduced UNC13A cryptic exon levels to 42.0%. A 50 nM dose of SEQ ID NO: 5165 (GAAAGTGSCATGGAGSGTGCAAG with two spacers) reduced UNC13A cryptic exon levels to 54.0%.

As shown in Table 8, a 200 nM dose of SEQ ID NO: 5198 (ATCTACSCTTTTATCCATSCACACA with two spacers) reduced UNC13A cryptic exon levels to 44.5%. A 50 nM dose of SEQ ID NO: 5198 (ATCTACSCTTTTATCCATSCACACA with two spacers) reduced UNC13A cryptic exon levels to 45.5%.

As shown in Table 8, a 200 nM dose of SEQ ID NO: 1147 (ATCTACTCTTTTATCCATCCACACA with no spacers) reduced UNC13A cryptic exon levels to 46.4%. A 50 nM dose of SEQ ID NO: 1147 (ATCTACTCTTTTATCCATCCACACA with no spacers) reduced UNC13A cryptic exon levels to 69.7%.

As shown in Table 8, a 200 nM dose of SEQ ID NO: 3587 (ACACATCCATCCATCCATCCATT with no spacers) reduced UNC13A cryptic exon levels to 47.8%.

As shown in Table 8, a 200 nM dose of SEQ ID NO: 5202 (CTACTCTSTTATCCASCCACACA with two spacers) reduced UNC13A cryptic exon levels to 48.1%. A 50 nM dose of SEQ ID NO: 5202 (CTACTCTSTTATCCASCCACACA with two spacers) reduced UNC13A cryptic exon levels to 76.5%.

As shown in Table 8, a 200 nM dose of SEQ ID NO: 5199 (ATCTACTCSTTTATCCATSCACACA with two spacers) reduced UNC13A cryptic exon levels to 48.2%. A 50 nM dose of SEQ ID NO: 5199 (ATCTACTCSTTTATCCATSCACACA with two spacers) reduced UNC13A cryptic exon levels to 62.8%.

As shown in Table 8, a 200 nM dose of SEQ ID NO: 5192 (CTTTCAGSAATTCAASCACACAT with two spacers) reduced UNC13A cryptic exon levels to 49.6%. A 50 nM dose of SEQ ID NO: 5192 (CTTTCAGSAATTCAASCACACAT with two spacers) reduced UNC13A cryptic exon levels to 70.9%.

As shown in Table 8, a 200 nM dose of SEQ ID NO: 3774 (AAGTGTCATGGAGAGTGCAAGGG with no spacers) reduced UNC13A cryptic exon levels to 49.8%. A 50 nM dose of SEQ ID NO: 3774 (AAGTGTCATGGAGAGTGCAAGGG with no spacers) reduced UNC13A cryptic exon levels to 67.0%.

TABLE 8
Performance of Exemplary UNC13A AONs evaluated in human derived iPSC motor neurons

| | | QPCR potency | | QPCR potency | |
|---|---|---|---|---|---|
| | | 200 nM | 200 nM | 50 nM | 50 nM |
| | | Normali- | SD | Normali- | SD |
| | Oligonucleotide | zed | Normalized | zed | Normalized |
| | Sequence* (where S | UNC13A | UNC13A | UNC13A | UNC13A |
| SEQ ID | indicates presence | Cryptic | Cryptic | Cryptic | Cryptic |
| NO: | of a Spacer) (5′ → 3′) | Exon | Exon | Exon | Exon |
| 273 | GAGACATACCCAGACACAAACGGCC | 4.38 | 4.41 | 20.3 | 14.4 |
| 2831 | GTTCTTTCCAGGAAACCCAGGCA | 5.11 | 4.14 | 26 | 15.2 |
| 5156 | GTTCTTTSCAGGAAASCCAGGCA | 6.62 | 6.06 | 41.2 | 15.2 |
| 5140 | GCAGCTGGSAGAGACATASCCAGAC | 8.3 | 7.86 | 31 | 26.4 |
| 303 | GTTCTTTCCAGGAAACCCAGGCAGC | 8.84 | 7.85 | 35.4 | 22.4 |
| 5135 | GCAGCTSGAAGAGACATASCCAGAC | 11.8 | 10.7 | 30.6 | 13 |
| 283 | GCAGCTGGAAGAGACATACCCAGAC | 13.8 | 7.11 | 66 | 45.9 |
| 2830 | TTCTTTCCAGGAAACCCAGGCAG | 15.2 | 7.39 | 34.4 | 25.1 |
| 3585 | ACATCCATCCATCCATCCATTCA | 15.8 | 8.84 | 45.4 | 14 |
| 5155 | TCTTTCCSGGAAACCSAGGCAGC | 18.2 | 9.18 | 41.9 | 24.2 |
| 5157 | TTCTTTCSAGGAAACSCAGGCAG | 20.1 | 9.33 | 42.8 | 27.7 |
| 1059 | ACACATCCATCCATCCATCCATTCA | 23.1 | 15.7 | 50.2 | 21.5 |
| 2829 | TCTTTCCAGGAAACCCAGGCAGC | 25 | 11.7 | 40.6 | 22.9 |
| 269 | CATACCCAGACACAAACGGCCCAAT | 27.2 | 20.6 | 43.4 | 28 |
| 1142 | CTCTTTTATCCATCCACACACCCAC | 34.4 | 20.1 | 64.3 | 34.7 |
| 3670 | CTCTTTTATCCATCCACACACCC | 40.5 | 15.2 | 82.2 | 45.5 |
| 301 | TCTTTCCAGGAAACCCAGGCAGCTG | 41.6 | 30.5 | 54.2 | 20.8 |
| 5165 | GAAAGTGSCATGGAGSGTGCAAG | 42 | 38.1 | 54 | 32.5 |
| 5198 | ATCTACSCTTTTATCCATSCACACA | 44.5 | 22.4 | 45.5 | 34.4 |
| 1147 | ATCTACTCTTTTATCCATCCACACA | 46.4 | 29.9 | 69.7 | 47.6 |
| 3587 | ACACATCCATCCATCCATCCATT | 47.8 | 24.9 | 122 | 41.9 |
| 5202 | CTACTCTSTTATCCASCCACACA | 48.1 | 35.4 | 76.5 | 37.9 |
| 5199 | ATCTACTCSTTTATCCATSCACACA | 48.2 | 21.5 | 62.8 | 44.3 |
| 5192 | CTTTCAGSAATTCAASCACACAT | 49.6 | 39.2 | 70.9 | 46.3 |
| 3774 | AAGTGTCATGGAGAGTGCAAGGG | 49.8 | 39.3 | 67 | 59.7 |
| 3586 | CACATCCATCCATCCATCCATTC | 51.6 | 14.2 | 80.3 | 41.4 |
| 248 | CAATCCTGAGTGGTTAGGGCTGGAA | 54.2 | 18.8 | 59.6 | 16.2 |
| 263 | CAGACACAAACGGCCCAATCCTGAG | 54.4 | 19.2 | 73.2 | 43.7 |
| 3668 | CTTTTATCCATCCACACACCCAC | 55.4 | 30.5 | 59 | 34.2 |
| 3669 | TCTTTTATCCATCCACACACCCA | 55.7 | 29.2 | 61.1 | 45.6 |
| 5196 | CTCTTTTSTCCATCCSCACACCC | 55.9 | 29.8 | 83.8 | 50.9 |
| 2562 | GGGCAGGCAGGAATGGTGAGTGG | 56.6 | 41 | 73.6 | 47.2 |
| 5141 | TCCACCAGSCACTCACAASCATCCA | 58 | 17.7 | 86.1 | 36.5 |
| 1124 | CACCCACCCATCTAACTACCCCAAA | 58.2 | 42.7 | 91 | 34.4 |
| 2561 | GGCAGGCAGGAATGGTGAGTGGA | 59.1 | 43.9 | 57.9 | 41.1 |
| 5195 | TCTTTTASCCATCCASACACCCA | 63.1 | 23.5 | 54.3 | 40.7 |
| 3652 | CACCCACCCATCTAACTACCCCA | 63.1 | 44.6 | 74.3 | 51.2 |
| 5166 | AAAGTGTSATGGAGASTGCAAGG | 63.7 | 39.2 | 55.5 | 53 |
| 5177 | TGCAATASTTCAACCSCACATCC | 64.1 | 25.7 | 75.7 | 42.9 |
| 5138 | GGGCAGGCSGGAATGGTGSGTGGAA | 64.2 | 32 | 73 | 39 |
| 34 | GGGCAGGCAGGAATGGTGAGTGGAA | 64.6 | 42.2 | 91.2 | 48.4 |
| 5145 | GGCAGGCSGGAATGGSGAGTGGA | 64.6 | 49.5 | 92.8 | 65 |
| 5193 | CTCTTTSATCCATCCACASACCCAC | 65.1 | 34.1 | 53 | 32.2 |
| 3775 | AAAGTGTCATGGAGAGTGCAAGG | 65.3 | 45.4 | 71.2 | 64.2 |
| 5186 | CAGTAATSCAACCACSCATCCAT | 65.5 | 23.7 | 92.7 | 24.1 |
| 5194 | CTCTTTTASCCATCCACASACCCAC | 65.6 | 37.8 | 67.8 | 29.5 |
| 3673 | CTACTCTTTTATCCATCCACACA | 67.1 | 32 | 50.5 | 34 |
| 1078 | ACCTTTCAGTAATTCAACCACACAT | 67.5 | 52.3 | 87.4 | 58 |
| 3776 | GAAAGTGTCATGGAGAGTGCAAG | 68.3 | 52.7 | 52.7 | 38.7 |
| 5197 | CTTTTATSCATCCACSCACCCAC | 68.7 | 58.9 | 59.2 | 33.2 |
| 5189 | ACCTTTCASTAATTCAACSACACAT | 69.2 | 35.5 | 115 | 79.8 |
| 5153 | ACACAAASGGCCCAASCCTGAGT | 69.3 | 54.9 | 114 | 67.4 |
| 5162 | CACCCACSCATCTAASTACCCCA | 71.4 | 65.7 | 111 | 77.3 |
| 5185 | AGTAATTSAACCACASATCCATC | 72 | 16 | 58 | 21.7 |
| 5164 | AAGTGTCSTGGAGAGSGCAAGGG | 72.1 | 51.9 | 70.7 | 59.6 |
| 2788 | ACACAAACGGCCCAATCCTGAGT | 72.4 | 18.3 | 99.8 | 34.6 |
| 3386 | TGCAATAGTTCAACCACACATCC | 74 | 35.8 | 124 | 53.6 |
| 3604 | CTTTCAGTAATTCAACCACACAT | 74.1 | 39.5 | 65.7 | 57.3 |
| 260 | ACACAAACGGCCCAATCCTGAGTGG | 74.3 | 16.6 | 109 | 53.2 |
| 244 | CCTGAGTGGTTAGGGCTGGAATAGA | 75.2 | 42.7 | 92.4 | 35.7 |
| 3605 | CCTTTCAGTAATTCAACCACACA | 76.2 | 42.9 | 145 | 138 |
| 2787 | CACAAACGGCCCAATCCTGAGTG | 76.8 | 37.3 | 91.2 | 46.8 |
| 3651 | ACCCACCCATCTAACTACCCCAA | 77.1 | 52.6 | 87.2 | 59.1 |
| 5200 | TCTACTCSTTTATCCSTCCACAC | 78.2 | 43.6 | 88.6 | 71.7 |
| 5154 | CACAAACSGCCCAATSCTGAGTG | 78.2 | 45.6 | 86.8 | 45.4 |
| 5152 | ACAAACGSCCCAATCSTGAGTGG | 78.3 | 38.8 | 66.1 | 35 |
| 5136 | TCCACCSGCCACTCACAASCATCCA | 78.7 | 73.6 | 120 | 50.9 |
| 3650 | CCCACCCATCTAACTACCCCAAA | 78.9 | 50.3 | 71.8 | 60.4 |
| 5144 | GGGCAGGSAGGAATGSTGAGTGG | 82.7 | 67.3 | 142 | 100 |
| 5173 | CTTGCASTAGTTCAACCASACATCC | 86 | 34.4 | 140 | 132 |
| 5201 | ATCTACTSTTTTATCSATCCACA | 86.3 | 55.6 | 117 | 68.8 |
| 254 | ACGGCCCAATCCTGAGTGGTTAGGG | 86.9 | 44.1 | 108 | 78 |
| 3547 | GATCCGCAACTTAATCCACCTAC | 88.3 | 76.5 | 115 | 86.5 |
| 5137 | GAAAGTSTCATGGAGAGTSCAAGGG | 89.6 | 50.5 | 107 | 42.8 |
| 3675 | ATCTACTCTTTTATCCATCCACA | 91.1 | 60.9 | 89.8 | 70.4 |
| 2786 | ACAAACGGCCCAATCCTGAGTGG | 92.9 | 43.5 | 78.5 | 47.3 |
| 2560 | GCAGGCAGGAATGGTGAGTGGAA | 94 | 74.1 | 118 | 82.7 |
| 5191 | ACCTTTCSGTAATTCSACCACAC | 95.2 | 44.9 | 60.6 | 52.1 |
| 112 | AGGTAAAGGGAATGGCATGGCGGGC | 96 | 49.3 | 115 | 57 |
| 3545 | TCCGCAACTTAATCCACCTACCC | 96 | 82.6 | 111 | 77.3 |
| 1248 | GAAAGTGTCATGGAGAGTGCAAGGG | 96.3 | 58.9 | 118 | 77.8 |
| 5176 | CTTGCAASAGTTCAASCACACAT | 98.9 | 42.4 | 90.8 | 40.8 |
| 3388 | CTTGCAATAGTTCAACCACACAT | 99.5 | 39 | 95.7 | 46.1 |
| 5161 | CCCACCCSTCTAACTSCCCCAAA | 99.7 | 71.7 | 112 | 82.8 |
| 5184 | CAGTAATTSAACCACACASCCATCC | 101 | 95.2 | 128 | 130 |
| 3387 | TTGCAATAGTTCAACCACACATC | 102 | 54.4 | 138 | 55.5 |
| 3606 | ACCTTTCAGTAATTCAACCACAC | 105 | 105 | 120 | 108 |
| 5187 | GTAATTCSACCACACSTCCATCC | 106 | 60.1 | 116 | 48.6 |
| 200 | GTAGTGAGAGTCAACCTGGAGGCCA | 107 | 21.8 | 92.6 | 27.2 |
| 3674 | TCTACTCTTTTATCCATCCACAC | 107 | 71.4 | 83.2 | 56.9 |
| 1260 | CACCATGGGAGAGAAAGTGTCATGG | 108 | 103 | 67.4 | 60.1 |
| 127 | AGGGAGGAGTTTTCCAGGTAAAGGG | 109 | 50.5 | 95.7 | 21.4 |
| 5143 | GCAGGCASGAATGGTSAGTGGAA | 109 | 74.8 | 150 | 98.2 |
| 5163 | ACCCACCSATCTAACSACCCCAA | 112 | 74.4 | 81.6 | 45.8 |
| 5178 | ACACATSCATCCATCCATSCATTCA | 114 | 36.7 | 135 | 39.5 |
| 5175 | TTGCAATSGTTCAACSACACATC | 115 | 54.7 | 98.6 | 51.2 |
| 5134 | AGGGAGSAGTTTTCCAGGSAAAGGG | 116 | 30 | 110 | 29.8 |
| 5182 | ACATCCASCCATCCASCCATTCA | 116 | 50.6 | 111 | 38 |
| 5188 | ACCTTTSAGTAATTCAACSACACAT | 116 | 81.4 | 101 | 74.7 |
| 5142 | GAAAGTGTSATGGAGAGTSCAAGGG | 117 | 40.6 | 115 | 66.2 |
| 5139 | AGGGAGGASTTTTCCAGGSAAAGGG | 118 | 30.4 | 111 | 22.7 |
| 195 | GAGAGTCAACCTGGAGGCCAAGGTG | 118 | 43.8 | 96.1 | 17.9 |
| 3379 | GTTCAACCACACATCCTTCCATT | 119 | 70.8 | 186 | 142 |
| 860 | CTTGCAATAGTTCAACCACACATCC | 120 | 57.3 | 140 | 70.5 |
| 927 | GTCTGTCCACCAGCCACTCACAACC | 120 | 104 | 154 | 122 |
| 3380 | AGTTCAACCACACATCCTTCCAT | 121 | 55 | 161 | 77.7 |
| 5169 | AGTTCAACSACACATCCTSCCATTC | 122 | 71.7 | 129 | 96.7 |
| 5181 | ACACATCSATCCATCSATCCATT | 123 | 40.6 | 108 | 40.9 |
| 3378 | TTCAACCACACATCCTTCCATTC | 124 | 9.97 | 143 | 48.5 |
| 5151 | GAGGAGGSGACCCTGSATCTGGA | 124 | 33.6 | 117 | 67.2 |
| 2639 | GGTAAAGGGAATGGCATGGCGGG | 125 | 50.2 | 110 | 68.5 |
| 5171 | AGTTCAASCACACATSCTTCCAT | 125 | 81.5 | 142 | 155 |
| 5180 | CACATCCSTCCATCCSTCCATTC | 128 | 67 | 89.1 | 32 |
| 5147 | AGGTAAASGGAATGGSATGGCGG | 128 | 77.5 | 149 | 85.4 |
| 5133 | GGGCAGSCAGGAATGGTGSGTGGAA | 129 | 72.2 | 105 | 58.4 |
| 5159 | GATCCGCSACTTAATSCACCTAC | 136 | 111 | 152 | 102 |
| 3598 | GTAATTCAACCACACATCCATCC | 137 | 54 | 107 | 52.1 |
| 5150 | AGAGGAGSTGACCCTSAATCTGG | 139 | 55.6 | 118 | 48.1 |
| 5170 | GTTCAACSACACATCSTTCCATT | 145 | 80.5 | 138 | 84.8 |
| 3600 | CAGTAATTCAACCACACATCCAT | 146 | 47.3 | 119 | 22.7 |
| 5160 | ATCCGCASCTTAATCSACCTACC | 147 | 63.2 | 132 | 76.5 |
| 2640 | AGGTAAAGGGAATGGCATGGCGG | 147 | 82.1 | 148 | 92.8 |
| 5158 | TCCGCAASTTAATCCSCCTACCC | 148 | 97 | 180 | 93.3 |
| 5149 | AGGAGGTSACCCTGASTCTGGAC | 149 | 47.3 | 101 | 58.2 |
| 5168 | AGTTCASCCACACATCCTSCCATTC | 149 | 79 | 132 | 60.8 |
| 5183 | CAGTAASTCAACCACACASCCATCC | 149 | 84.3 | 116 | 53.3 |
| 852 | AGTTCAACCACACATCCTTCCATTC | 151 | 106 | 137 | 114 |
| 5148 | GGTAAAGSGAATGGCSTGGCGGG | 151 | 111 | 106 | 63.6 |
| 1072 | CAGTAATTCAACCACACATCCATCC | 155 | 85.5 | 133 | 58.7 |
| 5172 | TTCAACCSCACATCCSTCCATTC | 156 | 159 | 139 | 80.1 |
| 5174 | CTTGCAATSGTTCAACCASACATCC | 157 | 99.7 | 123 | 77 |
| 42 | GAAGAGCTGGGCAGGCAGGAATGGT | 158 | 99.8 | 160 | 79.5 |
| 1019 | GATCCGCAACTTAATCCACCTACCC | 161 | 73 | 154 | 72 |
| 5179 | ACACATCCSTCCATCCATSCATTCA | 163 | 45.7 | 163 | 44.5 |
| 203 | TGAGTAGTGAGAGTCAACCTGGAGG | 164 | 54.7 | 108 | 59.6 |
| 101 | ATGGCATGGCGGGCAGTCTCAGAGG | 168 | 99.2 | 166 | 76.5 |
| 3599 | AGTAATTCAACCACACATCCATC | 173 | 58.2 | 107 | 51.4 |
| 922 | TCCACCAGCCACTCACAACCATCCA | 173 | 134 | 84.3 | 45.9 |
| 2688 | AGGAGGTGACCCTGAATCTGGAC | 176 | 46.2 | 85.1 | 44.1 |
| 454 | CACGAATCTACCCACCAACTCATCC | 178 | 116 | 140 | 49.4 |
| 2689 | GAGGAGGTGACCCTGAATCTGGA | 182 | 60.1 | 108 | 36.8 |
| 5190 | CCTTTCASTAATTCASCCACACA | 182 | 192 | 114 | 61.7 |
| 66 | TGTGGATGGTGTGGCCAGAAAGAGG | 185 | 70.1 | 156 | 70.7 |
| 3546 | ATCCGCAACTTAATCCACCTACC | 186 | 58.2 | 149 | 63.5 |
| 5146 | GTAAAGGSAATGGCASGGCGGGC | 193 | 116 | 161 | 79.1 |
| 930 | TTCGTCTGTCCACCAGCCACTCACA | 193 | 125 | 119 | 81.2 |
| 937 | TCCATTTTTCGTCTGTCCACCAGCC | 196 | 122 | 113 | 51.4 |
| 2690 | AGAGGAGGTGACCCTGAATCTGG | 197 | 67.6 | 108 | 41.2 |
| 162 | AGAGGAGGTGACCCTGAATCTGGAC | 197 | 92.2 | 119 | 37.6 |
| 77 | GGCCAGGAGAGTGTGGATGGTGTGG | 200 | 74.8 | 162 | 83.3 |
| 2638 | GTAAAGGGAATGGCATGGCGGGC | 205 | 79.8 | 80 | 52 |
| 115 | TCCAGGTAAAGGGAATGGCATGGCG | 215 | 102 | 149 | 127 |
| 108 | AAAGGGAATGGCATGGCGGGCAGTC | 226 | 99.9 | 178 | 113 |
| 277 | GGAAGAGACATACCCAGACACAAAC | 0.02 | 0.00 | 11.43 | 6.56 |
| 282 | CAGCTGGAAGAGACATACCCAGACA | 0.03 | 0.02 | 16.20 | 7.83 |
| 276 | GAAGAGACATACCCAGACACAAACG | 0.94 | 1.59 | 0.61 | 1.01 |
| 306 | AGAGTTCTTTCCAGGAAACCCAGGC | 1.47 | 2.28 | 19.61 | 9.44 |
| 307 | AAGAGTTCTTTCCAGGAAACCCAGG | 3.01 | 2.16 | 21.95 | 5.43 |
| 305 | GAGTTCTTTCCAGGAAACCCAGGCA | 3.49 | 2.07 | 29.80 | 2.76 |
| 304 | AGTTCTTTCCAGGAAACCCAGGCAG | 3.62 | 3.79 | 38.61 | 18.71 |
| 285 | AGGCAGCTGGAAGAGACATACCCAG | 4.63 | 5.37 | 32.99 | 2.70 |
| 274 | AGAGACATACCCAGACACAAACGGC | 5.98 | 6.18 | 12.51 | 6.77 |
| 279 | CTGGAAGAGACATACCCAGACACAA | 6.26 | 10.82 | 58.24 | 12.87 |
| 275 | AAGAGACATACCCAGACACAAACGG | 9.32 | 5.24 | 16.02 | 3.73 |
| 272 | AGACATACCCAGACACAAACGGCCC | 12.07 | 2.14 | 29.94 | 15.81 |
| 284 | GGCAGCTGGAAGAGACATACCCAGA | 12.92 | 4.64 | 50.87 | 17.93 |
| 286 | CAGGCAGCTGGAAGAGACATACCCA | 14.04 | 2.17 | 32.22 | 17.05 |
| 280 | GCTGGAAGAGACATACCCAGACACA | 15.74 | 19.34 | 36.35 | 14.55 |
| 281 | AGCTGGAAGAGACATACCCAGACAC | 18.82 | 10.27 | 20.31 | 17.71 |
| 283 | GCAGCTGGAAGAGACATACCCAGAC | 18.87 | 22.30 | 55.59 | 34.27 |
| 271 | GACATACCCAGACACAAACGGCCCA | 19.96 | 11.08 | 28.25 | 4.42 |
| 273 | GAGACATACCCAGACACAAACGGCC | 21.01 | 9.29 | 26.91 | 8.72 |
| 1057 | ACATCCATCCATCCATCCATTCATC | 21.36 | 5.97 | 63.61 | 8.59 |
| 1058 | CACATCCATCCATCCATCCATTCAT | 23.52 | 1.80 | 108.40 | 54.14 |
| 287 | CCAGGCAGCTGGAAGAGACATACCC | 25.02 | 3.34 | 60.96 | 5.29 |
| 1055 | ATCCATCCATCCATCCATTCATCCA | 25.59 | 10.98 | 86.98 | 36.11 |
| 1059 | ACACATCCATCCATCCATCCATTCA | 27.76 | 4.11 | 72.26 | 37.32 |
| 302 | TTCTTTCCAGGAAACCCAGGCAGCT | 30.66 | 1.40 | 86.91 | 11.41 |
| 301 | TCTTTCCAGGAAACCCAGGCAGCTG | 31.13 | 5.45 | 52.43 | 7.09 |
| 1056 | CATCCATCCATCCATCCATTCATCC | 35.45 | 4.51 | 75.99 | 10.37 |
| 1060 | CACACATCCATCCATCCATCCATTC | 38.25 | 19.28 | 96.63 | 22.67 |
| 270 | ACATACCCAGACACAAACGGCCCAA | 38.57 | 4.68 | 44.89 | 1.89 |
| 1062 | ACCACACATCCATCCATCCATCCAT | 39.40 | 7.68 | 71.32 | 18.18 |
| 1061 | CCACACATCCATCCATCCATCCATT | 39.80 | 8.88 | 77.72 | 2.82 |
| 1063 | AACCACACATCCATCCATCCATCCA | 41.07 | 10.10 | 97.41 | 24.31 |
| 300 | CTTTCCAGGAAACCCAGGCAGCTGG | 42.16 | 22.26 | 77.70 | 9.85 |
| 299 | TTTCCAGGAAACCCAGGCAGCTGGA | 51.84 | 9.47 | 72.21 | 10.14 |
| 269 | CATACCCAGACACAAACGGCCCAAT | 60.89 | 6.26 | 84.01 | 23.09 |

*Unless otherwise noted, each of the nucleosides of antisense oligonucleotides shown are modified nucleosides with 2′-O-(2-methoxyethyl) (2′-MOE) sugar moieties, each “C” is replaced with a 5-methylcytosine (5-MeC), and all internucleoside linkages are phosphorothioate linkages.
A spacer as indicated by S is not a nucleoside. S represents a spacer of Formula (Iia′) as disclosed herein.

### Example 4: Methods for Evaluating UNC13A Antisense Oligonucleotides

UNC13A antisense oligonucleotides were evaluated in iPSC derived human motor neurons (hMN). The cells were seeded in 96-well plates at a density of 40,000 cells/well. Antisense oligonucleotide (AON) to TDP43 was transfected with Endoporter (Gene Tools, Philomath, OR, USA) to decrease expression of the full length UNC13A transcript and increase expression of UNC13A cryptic exon. Vehicle control consisted of motor neuron treatment with Endoporter alone. Positive controls included cells that were treated with TDP43 AON alone (“AON TDP43” or “TDP43 AON”).

TDP43 AON is a gapmer oligonucleotide and has the following sequence and chemistry: 5′ A*A*G*G*C*T*T*C*A*T*A*T*T*G*T*A*C*T*T*T 3′ (SEQ ID NO: 5167) where *=phosphorothioate, underlined=DNA, other=2′MOE RNA; each “C” is 5-MeC.

To evaluate UNC13A AON ability to reduce UNC13A cryptic exon levels, antisense oligonucleotides to UNC13A were co-incubated with TDP43 AON in Endoporter in media before addition to the cells. After 72 hours, antisense oligonucleotides and Endoporter were washed out and replaced with fresh media alone. After six additional days, RNA was collected from the 96-well plates for RT-qPCR. RNA was isolated, cDNA generated and multiplexed RT-qPCR assay performed with Taqman probes for UNC13A cryptic exon, and reference GAPDH quantification.

Transcript levels (e.g., UNC13A cryptic exon, and TDP43 transcript) were detected by RT-qPCR using Taqman. Specifically, RT-qPCR was performed for detecting GAPDH using Thermofisher® TaqMan Gene Expression Assay Hs03929097_g1. UNC13a cryptic exon was detected using custom sequences.

### UNC13a Cryptic Exon:

Forward Primer:
(SEQ ID NO: 5203)
ATTGTTCTGCACGTCGGT
Reverse Primer:
(SEQ ID NO: 5204)
GTCTGGGTATGTCTCTTCCAG
Probe Sequence:
(SEQ ID NO: 5203)
AGTTCTTTCCAGGAAACCCAGGCA

To evaluate UNC13A AON ability to reduce UNC13A correctly spliced (CS)exon junction 20/21 levels, antisense oligonucleotides to UNC13A were co-incubated with TDP43 AON in Endoporter in media before addition to the cells. After 72 hours, antisense oligonucleotides and Endoporter were washed out and replaced with fresh media alone. After six additional days, RNA was collected from the 96-well plates for RT-qPCR. RNA was isolated, cDNA generated and multiplexed RT-qPCR assay performed with Taqman probes for UNC13A CS exon 20/21 junction, and reference GAPDH quantification.

Transcript levels (e.g., UNC13A exon junction 20/21, and TDP43 transcript) were detected by RT-qPCR using Taqman. Specifically, RT-qPCR was performed for detecting GAPDH using Thermofisher® TaqMan Gene Expression Assay Hs03929097_g1. UNC13a exon 20/21 junction was detect using TaqMan Gene Expression Assay Hs01000584_ml.

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

UNC13A-cryptic (Ct) was normalized to GAPDH (deltaCt). To visualize the quantitative changes (e.g., % decrease of UNC13A-cryptic), the normalized UNC13A-cryptic signal was further normalized to the vehicle (treated with Endoporter alone, deltadeltaCt).

Relative quantity (RQ) of transcript level was calculated using the equation RQ=2^{-deltadeltaCt} and is used to describe the treatment condition comparison to normal, healthy levels (1.0). RQ values for UNC13A cryptic were normalized using the following formula:

(((RQAON−RQendo)/(RQTDP43−RQendo))*100

Table 9 shows the RT-qPCR results of UNC13A AONs with spacers and performance of UNC13A AONs without spacers in human motor neurons.

As shown in Table 9, UNC13A AONs (e.g., UNC13A oligonucleotides without spacers or with one or two spacers) were tested for their ability to reduce UNC13A transcripts with a cryptic exon. In some cases, UNC13A AONs with spacers reduced UNC13A cryptic exon levels. In some cases, UNC13A AONs without spacers reduced UNC13A cryptic exon levels. Specific AON sequences are labeled according to their corresponding SEQ ID NO.

Correctly spliced UNC13A (Ct) was also normalized to GAPDH (deltaCt). To visualize the quantitative changes (e.g., % increase of correctly spliced UNC13A), the normalized correctly spliced UNC13A signal was further normalized to the vehicle (treated with Endoporter alone, deltadeltaCt).

Relative quantity (RQ) of transcript level was calculated using the equation RQ=2\hat{ }(−deltadeltaCt) and is used to describe the treatment condition comparison to normal, healthy levels (1.0). RQ values for UNC13A corrected splicing were normalized using the following formula:

$$( ( ( RQAON - RQTDP 43 ) / ( R Qendo - RQTDP 43 ) ) *$$

TABLE 9
Performance of Exemplary UNC13A AONs evaluated in human derived iPSC motor neurons

| | | UNC13A Corrected | | | | UNC13A Cryptic | | | |
|---|---|---|---|---|---|---|---|---|---|
| | | Splicing | | | | Splicing | | | |
| SEQ | Oligonucleotide | Normalized RQ | | | | Normalized RQ | | | |
| ID | Sequence* | 200nM | | 50nM | | 200nM | | 50nM | |
| NO: | (5′ to 3′) | Mean | STD | Mean | STD | Mean | STD | Mean | STD |
| 276 | GAAGAGACATACCCAGACACAA | 41.82 | 18.84 | −27.56 | 73.08 | 3.16 | 1.03 | 18.87 | 12.38 |
| | ACG | | | | | | | | |
| 2804 | GAAGAGACATACCCAGACACAA | 8.93 | 7.32 | −7.13 | 7.81 | 15.78 | 7.41 | 42.31 | 31.11 |
| | A | | | | | | | | |
| 2803 | AAGAGACATACCCAGACACAAA | −28.45 | 72.83 | −12.62 | 12.84 | 12.06 | 9.62 | 57.27 | 34.91 |
| | C | | | | | | | | |
| 2802 | AGAGACATACCCAGACACAAAC | 13.03 | 15.19 | 9.48 | 11.41 | 14.78 | 6.73 | 61.44 | 25.31 |
| | G | | | | | | | | |
| 2801 | GAGACATACCCAGACACAAACG | 28.03 | 29.67 | 7.69 | 35.17 | 18.53 | 10.52 | 33.25 | 20.41 |
| | G | | | | | | | | |
| 2800 | AGACATACCCAGACACAAACGG | 24.21 | 33.06 | 23.24 | 30.63 | 29.74 | 33.32 | 50.76 | 39.49 |
| | C | | | | | | | | |
| 2799 | GACATACCCAGACACAAACGGC | 2.47 | 35.11 | 2.89 | 26.90 | 26.29 | 25.67 | 46.66 | 32.30 |
| | C | | | | | | | | |
| 285 | AGGCAGCTGGAAGAGACATACC | 47.19 | 57.85 | 15.52 | 30.16 | 2.07 | 3.30 | 13.45 | 13.12 |
| | CAG | | | | | | | | |
| 2813 | AGGCAGCTGGAAGAGACATACC | 37.80 | 13.72 | 1.51 | 9.33 | 2.32 | 3.41 | 22.31 | 14.52 |
| | C | | | | | | | | |
| 2812 | GGCAGCTGGAAGAGACATACCC | 39.83 | 25.50 | 19.20 | 21.74 | 7.01 | 5.73 | 25.13 | 28.23 |
| | A | | | | | | | | |
| 2811 | GCAGCTGGAAGAGACATACCCA | 43.08 | 23.32 | 4.48 | 7.87 | 5.11 | 6.91 | 32.17 | 23.76 |
| | G | | | | | | | | |
| 2810 | CAGCTGGAAGAGACATACCCAG | −21.34 | 16.48 | −144.30 | 28.86 | 7.59 | 6.87 | 21.20 | 16.74 |
| | A | | | | | | | | |
| 2809 | AGCTGGAAGAGACATACCCAGA | −173.70 | 29.80 | −75.73 | 8.72 | 4.20 | 7.60 | 165.20 | 96.22 |
| | C | | | | | | | | |
| 5253 | GCAGCTGSAAGAGACSTACCCA | 64.46 | 20.00 | −11.79 | 20.17 | 2.34 | 1.77 | 17.01 | 6.37 |
| | G | | | | | | | | |
| 5251 | CAGCTGGSAGAGACASACCCAG | 87.01 | 8.70 | 28.35 | 13.19 | 5.72 | 3.41 | 22.86 | 10.45 |
| | A | | | | | | | | |
| 5266 | GTTCTTSCCAGGAAACCCSGGC | −8.32 | 3.39 | −10.50 | 8.45 | 29.49 | 14.34 | 51.95 | 12.36 |
| | AGC | | | | | | | | |
| 5265 | GTTCTTTCSAGGAAACCCSGGC | 6.29 | 22.23 | 6.58 | 3.64 | 8.10 | 5.42 | 28.87 | 7.85 |
| | AGC | | | | | | | | |
| 5269 | AGAGTTCSTTCCAGGSAACCCA | 61.99 | 21.81 | −24.46 | 58.00 | 12.82 | 3.63 | 55.05 | 18.66 |
| | G | | | | | | | | |
| 5267 | GAGTTCTSTCCAGGASACCCAG | 38.47 | 29.10 | −14.70 | 48.97 | 17.11 | 11.96 | 80.63 | 36.03 |
| | G | | | | | | | | |
| 5268 | AGTTCTTSCCAGGAASCCCAGG | 9.90 | 25.86 | −7.31 | 26.47 | 26.33 | 9.18 | 92.35 | 32.38 |
| | C | | | | | | | | |
| 5271 | AGAGTTSTTTCCAGGAAASCCA | 44.52 | 32.61 | 57.40 | 14.54 | 9.44 | 4.83 | 22.79 | 6.99 |
| | GGC | | | | | | | | |
| 5270 | AGAGTTCTSTCCAGGAAASCCA | 44.46 | 8.40 | 48.23 | 22.36 | 3.31 | 2.76 | 14.18 | 10.22 |
| | GGC | | | | | | | | |
| 5243 | GAGACATSCCCAGACSCAAACG | 84.50 | 29.82 | 47.59 | 10.35 | 31.74 | 13.09 | 79.61 | 17.53 |
| | G | | | | | | | | |
| 5241 | AGACATASCCAGACASAAACGG | 90.95 | 10.72 | 22.20 | 40.15 | 30.38 | 13.73 | 82.12 | 14.48 |
| | C | | | | | | | | |
| 5242 | GACATACSCAGACACSAACGGC | 73.81 | 41.97 | 36.23 | 29.75 | 22.06 | 9.58 | 70.87 | 9.37 |
| | C | | | | | | | | |
| 5245 | GAGACASACCCAGACACASACG | 80.40 | 19.16 | 23.13 | 19.81 | 31.05 | 15.73 | 88.26 | 28.47 |
| | GCC | | | | | | | | |
| 5244 | GAGACATASCCAGACACASACG | 126.50 | 25.60 | 88.00 | 43.30 | 17.80 | 10.45 | 77.70 | 25.98 |
| | GCC | | | | | | | | |
| 5257 | AGGCAGSTGGAAGAGACASACC | 96.50 | 22.07 | 44.00 | 25.12 | 10.88 | 6.15 | 35.95 | 13.92 |
| | CAG | | | | | | | | |
| 5256 | AGGCAGCTSGAAGAGACASACC | 103.10 | 12.62 | 66.18 | 15.49 | 4.04 | 2.80 | 27.84 | 10.66 |
| | CAG | | | | | | | | |
| 5255 | AGGCAGCSGGAAGAGSCATACC | 92.83 | 15.27 | 58.96 | 6.90 | 12.26 | 4.78 | 31.13 | 9.49 |
| | C | | | | | | | | |
| 5254 | GGCAGCTSGAAGAGASATACCC | 82.43 | 18.79 | 58.45 | 12.96 | 8.83 | 8.20 | 37.74 | 10.12 |
| | A | | | | | | | | |
| 5252 | AGCTGGASGAGACATSCCCAGA | 87.57 | 8.14 | 9.59 | 32.49 | 7.43 | 3.22 | 28.79 | 15.38 |
| | C | | | | | | | | |
| 5250 | GAAGAGSCATACCCAGACSCAA | 102.70 | 14.83 | 54.28 | 31.30 | 29.53 | 14.04 | 75.86 | 28.78 |
| | ACG | | | | | | | | |
| 5249 | GAAGAGACSTACCCAGACSCAA | 99.18 | 24.63 | 46.29 | 25.42 | 22.36 | 7.32 | 75.43 | 35.92 |
| | ACG | | | | | | | | |
| 5248 | GAAGAGASATACCCASACACAA | 49.24 | 20.69 | 21.53 | 23.44 | 89.72 | 39.56 | 135.70 | 43.02 |
| | A | | | | | | | | |
| 5246 | AAGAGACSTACCCAGSCACAAA | 28.09 | 21.14 | 22.72 | 15.94 | 52.17 | 11.17 | 112.70 | 38.44 |
| | C | | | | | | | | |
| 5247 | AGAGACASACCCAGASACAAAC | 78.70 | 27.23 | 29.31 | 21.53 | 29.70 | 17.53 | 85.44 | 19.01 |
| | G | | | | | | | | |
| 5292 | GTT[LNA-C]TTTSCAGGAAAS | 25.91 | 16.04 | 21.89 | 19.68 | 4.99 | 12.69 | 17.06 | 44.28 |
| | CCA[LNA-G]GCA | | | | | | | | |
| 5291 | GTT[LNA-C]TTTSCAG[LNA- | 19.94 | 10.97 | 10.81 | 5.00 | 20.25 | 27.06 | 28.08 | 34.90 |
| | G]AAASCCA[LNA-G]GCA | | | | | | | | |
| 5290 | GTTCTT[LNA-T]SCAGGAA | 8.93 | 9.25 | 6.86 | 7.65 | −1.28 | 1.43 | 7.60 | 13.16 |
| | [LNA-A]SCCAGGCA | | | | | | | | |
| 5289 | GTTCTT[LNA-T]SCAGGAAA | 0.18 | 4.96 | 12.23 | 11.77 | −1.20 | 1.39 | 29.03 | 35.58 |
| | S[LNA-C]CAGGCA | | | | | | | | |
| 5288 | GTTCTTTS[LNA-C]AGGAAA | 14.39 | 10.91 | 10.40 | 7.95 | 6.29 | 12.97 | 21.83 | 31.86 |
| | S[LNA-C]CAGGCA | | | | | | | | |
| 5287 | GCA[LNA-G]CTGGSAGAGACA | −10.22 | 30.13 | 29.91 | 20.97 | 11.14 | 21.19 | 6.45 | 7.80 |
| | TASCC[LNA-A]GAC | | | | | | | | |
| 5286 | GCA[LNA-G]CTGGSAGA[LNA- | 10.40 | 16.54 | 5.17 | 18.78 | 12.36 | 10.45 | 32.23 | 21.90 |
| | G]ACATASCC[LNA-A]GAC | | | | | | | | |
| 5285 | GCAGCTG[LNA-G]SAGAGACA | 0.57 | 12.49 | −14.07 | 16.60 | 7.76 | 8.69 | 16.24 | 14.15 |
| | T[LNA-A]SCCAGAC | | | | | | | | |
| 5284 | GCAGCTG[LNA-G]SAGAGACA | −6.16 | 17.10 | 7.57 | 18.74 | 14.62 | 9.19 | 46.32 | 24.49 |
| | TAS[LNA-C]CAGAC | | | | | | | | |
| 5283 | GCAGCTGGS[LNA-A]GAGACA | 18.14 | 16.98 | 9.28 | 26.20 | 8.90 | 6.17 | 40.24 | 12.47 |
| | TAS[LNA-C]CAGAC | | | | | | | | |
| 5292 | GTT[LNA-C]TTTSCAGGAAAS | 4.16 | 16.50 | 4.89 | 11.13 | 15.39 | 21.60 | 19.66 | 21.58 |
| | CCA[LNA-G]GCA | | | | | | | | |
| 5291 | GTT[LNA-C]TTTSCAG[LNA- | −0.74 | 10.05 | 1.42 | 8.15 | 11.70 | 16.06 | 52.88 | 33.79 |
| | G]AAASCCA[LNA-G]GCA | | | | | | | | |
| 5290 | GTTCTT[LNA-T]SCAGGAA | −3.14 | 10.20 | 0.93 | 12.63 | 11.03 | 12.72 | 21.48 | 26.72 |
| | [LNA-A]SCCAGGCA | | | | | | | | |
| 5289 | GTTCTT[LNA-T]SCAGGAAA | 0.43 | 5.96 | 0.99 | 9.62 | 6.88 | 15.65 | 39.99 | 36.29 |
| | S[LNA-C]CAGGCA | | | | | | | | |
| 5288 | GTTCTTTS[LNA-C]AGGAAA | −6.06 | 6.61 | −11.46 | 10.35 | 6.58 | 11.97 | 20.12 | 30.75 |
| | S[LNA-C]CAGGCA | | | | | | | | |
| 5241 | AGACATASCCAGACASAAACGG | 37.10 | 22.45 | 53.64 | 15.42 | 43.13 | 22.30 | 79.66 | 21.62 |
| | C | | | | | | | | |
| 5242 | GACATACSCAGACACSAACGGC | 50.01 | 21.60 | 21.58 | 15.74 | 67.02 | 35.41 | 83.55 | 61.27 |
| | C | | | | | | | | |
| 5243 | GAGACATSCCCAGACSCAAACG | 49.95 | 11.51 | 29.51 | 6.61 | 40.47 | 25.79 | 143.30 | 44.64 |
| | G | | | | | | | | |
| 5237 | AGACATA(S1)CCAGACA(S1) | 41.00 | 21.58 | 27.91 | 8.85 | 51.69 | 49.87 | 103.79 | 37.66 |
| | AAACGGC | | | | | | | | |
| 5236 | GACATAC(S1)CAGACAC(S1) | 46.48 | 5.54 | 20.34 | 11.08 | 9.03 | 14.52 | 72.91 | 57.87 |
| | AACGGCC | | | | | | | | |
| 5235 | GAGACAT(S1)CCCAGAC(S1) | 45.33 | 8.89 | 33.99 | 13.59 | 23.89 | 18.73 | 133.34 | 42.50 |
| | CAAACGG | | | | | | | | |
| 5157 | TTCTTTCSAGGAAACSCAGGCA | 11.16 | 8.04 | 9.91 | 7.60 | 5.90 | 10.52 | 94.03 | 74.88 |
| | G | | | | | | | | |
| 5156 | GTTCTTTSCAGGAAASCCAGGC | 5.10 | 4.93 | −8.11 | 6.11 | 12.92 | 15.28 | 25.15 | 25.51 |
| | A | | | | | | | | |
| 5155 | TCTTTCCSGGAAACCSAGGCAG | 10.06 | 10.19 | 3.12 | 11.72 | 6.96 | 9.36 | 26.97 | 9.87 |
| | C | | | | | | | | |
| 5261 | TTCTTTC(S1)AGGAAAC(S1) | 11.38 | 11.69 | 7.69 | 15.12 | 11.99 | 14.32 | 54.16 | 41.27 |
| | CAGGCAG | | | | | | | | |
| 5260 | GTTCTTT(S1)CAGGAAA(S1) | 1.44 | 7.33 | −4.17 | 4.75 | 9.79 | 9.57 | 27.69 | 15.98 |
| | CCAGGCA | | | | | | | | |
| 5259 | TCTTTCC(S1)GGAAACC(S1) | 7.18 | 11.81 | −11.24 | 5.27 | 18.13 | 13.56 | 55.71 | 28.06 |
| | AGGCAGC | | | | | | | | |
| 5182 | ACATCCASCCATCCASCCATTC | −37.97 | 26.85 | −4.07 | 52.32 | 129.74 | 41.79 | 122.30 | 48.42 |
| | A | | | | | | | | |
| 5181 | ACACATCSATCCATCSATCCAT | 5.77 | 35.61 | −11.91 | 27.75 | 96.81 | 38.14 | 111.73 | 35.15 |
| | T | | | | | | | | |
| 5180 | CACATCCSTCCATCCSTCCATT | −14.00 | 37.66 | 1.96 | 30.50 | 159.36 | 43.66 | 164.55 | 47.07 |
| | C | | | | | | | | |
| 5274 | ACATCCA(S1)CCATCCA(S1) | −16.66 | 14.80 | −5.26 | 26.53 | 165.82 | 42.96 | 151.38 | 29.35 |
| | CCATTCA | | | | | | | | |
| 5273 | ACACATC(S1)ATCCATC(S1) | −7.44 | 24.85 | −21.89 | 16.44 | 162.48 | 39.49 | 116.38 | 15.71 |
| | ATCCATT | | | | | | | | |
| 5272 | CACATCC(S1)TCCATCC(S1) | −9.65 | 36.42 | −0.61 | 27.96 | 145.25 | 19.25 | 132.10 | 35.74 |
| | TCCATTC | | | | | | | | |
| 5209 | TCTTTCCAGGAAACCCAGGCA | 18.29 | 17.99 | 9.91 | 14.01 | 16.42 | 18.29 | 16.34 | 9.91 |
| 5220 | CAGGAAACCCAGGCA | 2.74 | 3.88 | 2.44 | 1.85 | 16.24 | 2.74 | 14.66 | 2.44 |
| 5221 | TCTTTCCAGGAAACC | 12.94 | 2.00 | 7.34 | 7.10 | 12.75 | 12.94 | 12.29 | 7.34 |
| 5218 | CCAGGAAACCCAGGCA | 0.00 | 0.00 | 0.74 | 1.05 | 13.49 | 0.00 | 16.04 | 0.74 |
| 5219 | TCTTTCCAGGAAACCC | 17.89 | 25.30 | 17.60 | 2.62 | 18.82 | 17.89 | 15.79 | 17.60 |
| 5216 | TCCAGGAAACCCAGGCA | 0.00 | 0.00 | 1.54 | 2.17 | 14.54 | 0.00 | 15.41 | 1.54 |
| 5217 | TCTTTCCAGGAAACCCA | 6.80 | 9.61 | 2.79 | 1.25 | 12.77 | 6.80 | 15.04 | 2.79 |
| 5214 | TTCCAGGAAACCCAGGCA | 0.70 | 0.98 | 0.00 | 0.00 | 16.31 | 0.70 | 11.42 | 0.00 |
| 5215 | TCTTTCCAGGAAACCCAG | 19.21 | 8.43 | 49.84 | 70.94 | 16.89 | 19.21 | 19.14 | 49.84 |
| 5212 | TTTCCAGGAAACCCAGGCA | 0.55 | 0.78 | 3.96 | 5.60 | 13.97 | 0.55 | 12.15 | 3.96 |
| 5213 | TCTTTCCAGGAAACCCAGG | 11.47 | 8.56 | 2.24 | 3.17 | 16.52 | 11.47 | 15.47 | 2.24 |
| 5210 | CTTTCCAGGAAACCCAGGCA | 6.60 | 0.75 | 1.59 | 2.25 | 18.60 | 6.60 | 12.55 | 1.59 |
| 5211 | TCTTTCCAGGAAACCCAGGC | 15.08 | 15.12 | 11.29 | 4.48 | 15.92 | 15.08 | 12.81 | 11.29 |

*Unless otherwise noted, each of the nucleosides of antisense oligonucleotides shown are modified nucleosides with 2′-O-(2-methoxyethyl) (2′-MOE) sugar moieties, each “C” is replaced with a 5-methylcytosine (5-MeC), and all internucleoside linkages are phosphorothioate linkages.
A spacer, as indicated by any of S, S1, or S#, is not a nucleoside. S represents a spacer of Formula (Iia′) as disclosed herein. S1 represents a spacer of Formula (IIIa′); S# represents a spacer of Formula (IIib′).
[LNA-X] refers to a locked nucleic acid with a corresponding base X (e.g., LNA-A refers to a locked nucleic acid with an adenine nucleobase, LNA-G refers to a locked nucleic acid with a guanine nucleobase, LNA-T refers to a locked nucleic acid with a thymine nucleobase, and LNA-C refers to a locked nucleic acid with a cytosine nucleobase).

### INCORPORATION BY REFERENCE

The entire disclosure of each of the patent documents and scientific articles cited herein is incorporated by reference for all purposes.

### EQUIVALENTS

The disclosure can be embodied in other specific forms without departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

What is claimed is:

1. A compound comprising a modified oligonucleotide comprising a sequence that is at least 85% complementary to an equal length portion of any one of SEQ ID NO: 5057-5065 or SEQ ID NOs: 5206-5208, a sequence having 90% identity thereof, or to a 15 to 50 contiguous nucleobase portion thereof, wherein at least one (i.e., one or more) nucleoside linkage of the oligonucleotide is a non-natural linkage.

2. The compound of claim 1, wherein the modified oligonucleotide comprises a spacer.

3. A compound comprising a modified oligonucleotide comprising a sequence that is at least 85% complementary to an equal length portion of any one of SEQ ID NO: 5057-5065 or SEQ ID NOs: 5206-5208, a sequence having 90% identity thereof, or to a 15 to 50 contiguous nucleobase portion thereof, wherein at least one (i.e., one or more) nucleoside linkage of the oligonucleotide is a non-natural linkage, and further wherein the oligonucleotide comprises a spacer.

4. An oligonucleotide comprising a sequence that is at least 85% complementary to an equal length portion of any one of SEQ ID NO: 5057-5065 or SEQ ID NOs: 5206-5208, a sequence having 90% identity thereof, or to a 15 to 50 contiguous nucleobase portion thereof, wherein at least one (i.e., one or more) nucleoside linkage of the oligonucleotide is a non-natural linkage.

5. The oligonucleotide of claim 4, further comprising a spacer.

6. An oligonucleotide comprising a sequence that is at least 85 98% complementary to an equal length portion of any one of SEQ ID NO: 5057-5065 or SEQ ID NOs: 5206-5208, a sequence having 90% identity thereof, or to a 15 to 50 contiguous nucleobase portion thereof, wherein at least one (i.e., one or more) nucleoside linkage of the oligonucleotide is a non-natural linkage, and further wherein the oligonucleotide comprises a spacer.

7. The compound of claims 1-3 or oligonucleotide of claims 5-6, wherein the oligonucleotide comprises a segment with at most 11 linked nucleosides.

8. The compound of claims 1-3 or 7, or oligonucleotides of claims 5-7, wherein the oligonucleotide comprises a segment with at most 10, 9, or 8 linked nucleosides.

9. The compound of any one of claims 1-3 or 7-8 or oligonucleotide of any one of claims 44-8, wherein the oligonucleotide comprises a segment with at most 7 linked nucleosides.

10. The compound of any one of claims 1-3 or 7-9 or oligonucleotide of any one of claims 5-9, wherein the oligonucleotide comprises a segment with at most 6, 5, 4, 3, or 2 linked nucleosides.

11. The compound of any one of claims 1-3 or 7-10 or oligonucleotide of any one of claims 5-10, wherein every segment of the oligonucleotide comprises at most 7 linked nucleosides.

12. The compound or oligonucleotide of any one of claims 7-11, wherein the oligonucleotide comprises a sequence that shares at least 85% identity with an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292.

13. The compound or oligonucleotide of any one of claims 7-12, wherein the oligonucleotide comprises a sequence that shares at least 90% identity with an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292.

14. The compound or oligonucleotide of any one of claims 7-13, wherein the oligonucleotide comprises a sequence that shares at least 95% identity with an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292.

15. The compound or oligonucleotide of any one of claims 7-13, wherein the oligonucleotide comprises a sequence that shares 100% identity with an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292.

16. The compound or oligonucleotide of any one of claims 7-13, wherein the oligonucleotide comprises a segment with at most 11 linked nucleosides or at most 7 linked nucleosides, and wherein the oligonucleotide comprises a sequence that shares at least 85% identity with an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292.

17. The compound or oligonucleotide of claim 16, wherein the oligonucleotide comprises a segment with at most 6, 5, 4, 3, or 2 linked nucleosides, and wherein the oligonucleotide comprises a sequence that shares at least 85% identity with an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292.

18. The compound or oligonucleotide of claim 16 or 17, wherein the oligonucleotide comprises a segment with at most 6, 5, 4, 3, or 2 linked nucleosides, and wherein the oligonucleotide comprises a sequence that shares at least 90% identity with an equal length portion of any one of SEQ ID NOs: 1-1264, SEQ ID NOs: 2529-3792, SEQ ID NOs: 5168-5202, SEQ ID NOs: 5209-5221, or SEQ ID NOs: 5235-5292.

19. The compound or oligonucleotide of any one of claims 1-18, wherein the oligonucleotide is at least 18, at least 19, at least 20, at least 21, at least 22, at least 23, at least 24, or at least 25 oligonucleotide units in length.

20. The compound or oligonucleotide of claim 19, wherein the oligonucleotide is at least 19 oligonucleotide units in length.

21. The compound or oligonucleotide of any one of claims 1-3 and 5-20, wherein the spacer is a nucleoside-replacement group comprising a non-sugar substitute that is incapable of linking to a nucleotide base.

22. The compound or oligonucleotide of claim 21, wherein the spacer is located between positions 10 and 15 of the oligonucleotide.

23. The compound or oligonucleotide of claim 21, wherein the spacer is located between positions 7 and 11 of the oligonucleotide.

24. The compound or oligonucleotide of claim 21 or 23, wherein the oligonucleotide further comprises a second spacer, wherein the second spacer is located between positions 14 and 22 of the oligonucleotide.

25. The compound or oligonucleotide of claim 24, wherein the spacer and the second spacer are separated by at least 5 nucleobases, at least 6 nucleobases, or at least 7 nucleobases in the oligonucleotide.

26. The compound or oligonucleotide of claim 24 or 25, wherein the spacer is located between positions 7 and 9 of the oligonucleotide, and wherein the second spacer is located between positions 15 and 18 of the oligonucleotide.

27. The compound or oligonucleotide of any one of claims 24-26, wherein the spacer is located at position 8 of the oligonucleotide, and wherein the second spacer is located at position 16 of the oligonucleotide.

28. The compound or oligonucleotide of any one of claims 24-27, wherein the oligonucleotide further comprises a third spacer, wherein the third spacer is located between positions 21 and 24 of the oligonucleotide.

29. The compound or oligonucleotide of claim 21, wherein the spacer is located between positions 2 and 5 of the oligonucleotide.

30. The compound or oligonucleotide of claim 29, wherein the oligonucleotide further comprises a second spacer, wherein the second spacer is located between positions 8 and 12 of the oligonucleotide.

31. The compound or oligonucleotide of claim 30, wherein the oligonucleotide further comprises a third spacer, wherein the third spacer is located between positions 18 and 22 of the oligonucleotide.

32. The compound or oligonucleotide of claim 21, wherein the oligonucleotide further comprises a second spacer and a third spacer, wherein the three spacers are located at positions in the oligonucleotide such that each segment of the oligonucleotide has at most 7 linked nucleosides.

33. The compound or oligonucleotide of claim 32, wherein at least two of the three spacers are adjacent to a guanine nucleobase.

34. The compound or oligonucleotide of claim 33, wherein each of the at least two of the three spacers immediately precede a guanine nucleobase.

35. The compound or oligonucleotide of any one of claims 21-34, wherein each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

36. The compound or oligonucleotide of any one of claims 21-34, wherein each of the first, second or third spacers is independently represented by Formula (X), wherein: 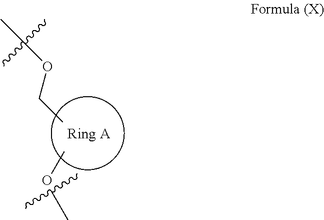
Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the 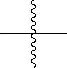
symbol represents the point of connection to an internucleoside linkage.

37. The compound or oligonucleotide of claim 36, wherein each of the first, second or third spacers is independently represented by Formula (Xa), wherein:

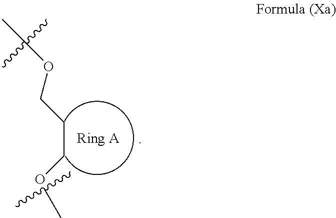

38. The compound or nucleotide of claim 36 or 37, wherein ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, 259pyrrolidinyl, piperidinyl, piperazinyl, morpholinyl and azepanyl.

39. The compound or nucleotide of claim 38 wherein ring A is tetrahydrofuranyl.

40. The compound or nucleotide of claim 38 wherein ring A is tetrahydropyranyl.

41. The compound or oligonucleotide of any one of claims 21-34, wherein each of the first, second or third spacers is independently represented by Formula I, wherein: 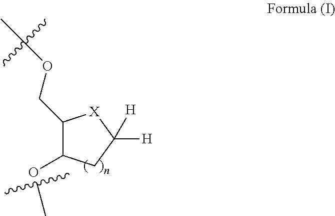
X is selected from —CH₂— and —O—; and
n is 0, 1, 2 or 3.

42. The compound or oligonucleotide of any one of claims 21-34, wherein each of the first, second or third spacers is independently represented by Formula I′, wherein: 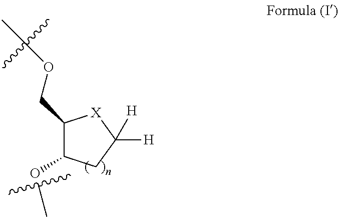
X is selected from —CH₂— and —O—; and
n is 0, 1, 2 or 3.

43. The compound or oligonucleotide of any one of claims 21-34, wherein each of the first, second or third spacers is independently represented by Formula (Ia), wherein: 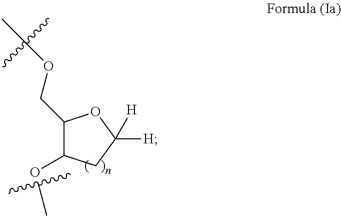
and
n is 0, 1, 2 or 3.

44. The compound or oligonucleotide of any one of claims 21-34, wherein each of the first, second or third spacers is independently represented by Formula (Ia′), wherein: 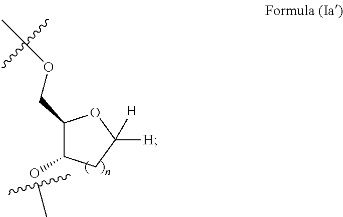
and
n is 0, 1, 2 or 3.

45. The compound or oligonucleotide of any one of claims 21-34, wherein each of the first, second or third spacers is independently represented by Formula II, wherein: 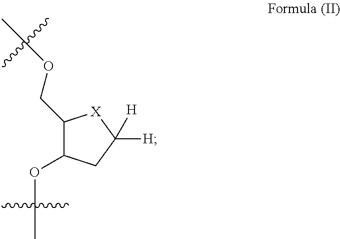
and
X is selected from —CH₂— and —O—.

46. The compound or oligonucleotide of any one of claims 21-34, wherein each of the first, second or third spacers is independently represented by Formula II′, wherein: 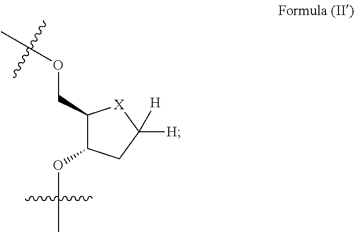
and
X is selected from —CH₂— and —O—.

47. The compound or oligonucleotide of any one of claims 21-34, wherein each of the first, second or third spacers is independently represented by Formula (Iia), wherein:

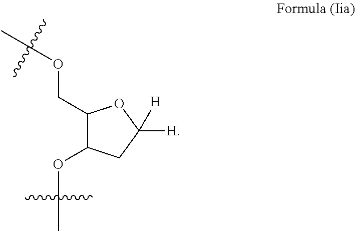

48. The compound or oligonucleotide of any one of claims 21-34, wherein each of the first, second or third spacers is independently represented by Formula (Iia′), wherein:

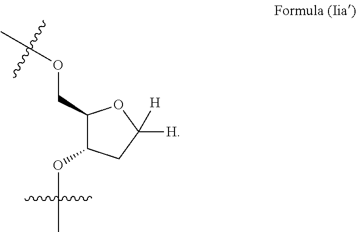

49. The compound or oligonucleotide of any one of claims 21-34, wherein each of the first, second or third spacers is independently represented by Formula (IIi), wherein: 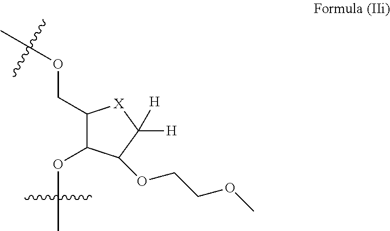
X is selected from —CH₂— and —O—.

50. The compound or oligonucleotide of any one of claims 21-34, wherein each of the first, second or third spacers is independently represented by Formula (IIi′), wherein: 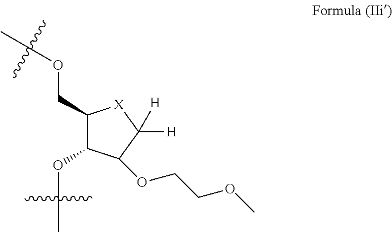
X is selected from —CH₂— and —O—.

51. The compound or oligonucleotide of any one of claims 21-34, wherein each of the first, second or third spacers is independently represented by Formula (IIib), wherein:

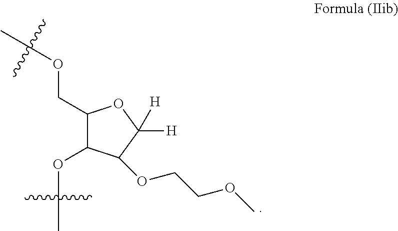 [SMILES: *OCC1OCC(OCCOC)C1O*]

52. The compound or oligonucleotide of any one of claims 21-34, wherein each of the first, second or third spacers is independently represented by Formula (IIib′), wherein:

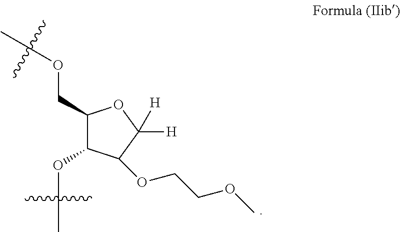 [SMILES: *OC[C@H]1OCC(OCCOC)[C@@H]1O*]

53. The compound or oligonucleotide of any one of claims 21-34, wherein each of the first, second or third spacers is independently represented by Formula III, wherein: 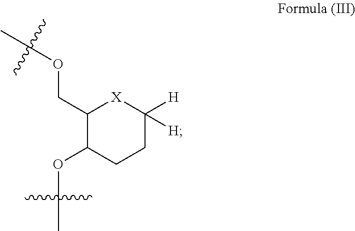
and
X is selected from —CH₂— and —O—.

54. The compound or oligonucleotide of any one of claims 21-34, wherein each of the first, second or third spacers is independently represented by Formula III′, wherein: 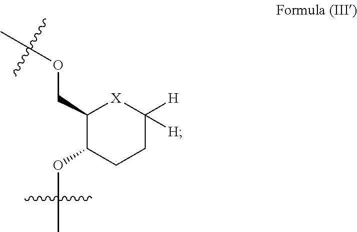
and
X is selected from —CH₂— and —O—.

55. The compound or oligonucleotide of any one of claims 21-34, wherein each of the first, second or third spacers is independently represented by Formula (IIIa), wherein:

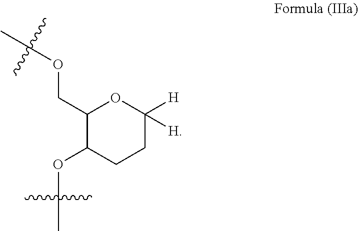

56. The compound or oligonucleotide of any one of claims 21-34, wherein each of the first, second or third spacers is independently represented by Formula (IIIa′), wherein:

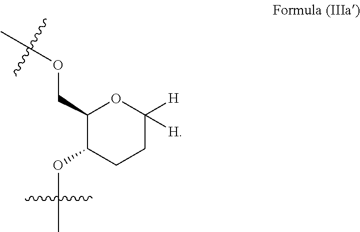

57. The compound or oligonucleotide of any one of the above claims, wherein the oligonucleotide further comprises a locked nucleic acid (LNA).

58. The compound or oligonucleotide of claim 57, wherein the locked nucleic acid (LNA) is located at one of positions 4, 7, 9, 12, 15, or 20 of the oligonucleotide.

59. The compound or oligonucleotide of any one of the above claims, wherein the oligonucleotide comprising the spacer has a GC content of at least 10%.

60. The compound or oligonucleotide of any one of the above claims, wherein the oligonucleotide comprising the spacer has a GC content of at least 20%.

61. The compound or oligonucleotide of any one of the above claims, wherein the oligonucleotide comprising the spacer has a GC content of at least 25%.

62. The compound or oligonucleotide of any one of the above claims, wherein the oligonucleotide comprising the spacer has a GC content of at least 30%.

63. The compound or oligonucleotide of any one of the above claims, wherein the oligonucleotide comprising the spacer has a GC content of at least 40%.

64. The compound or oligonucleotide of any one of the above claims, wherein the oligonucleotide comprising the spacer has a GC content of at least 50%.

65. The compound or oligonucleotide of any one of the above claims, wherein the oligonucleotide is between 12 and 40 oligonucleotide units in length.

66. The compound or oligonucleotide of any one of the above claims, wherein at least one (i.e., one or more) nucleoside linkage of the oligonucleotide is independently selected from the group consisting of a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

67. The compound or oligonucleotide of any one of claims 1-66, wherein one or more nucleoside linkages that link a base at position 3 or position 4 of the oligonucleotide are phosphodiester linkages.

68. The compound or oligonucleotide of claim 67, wherein only one nucleoside linkage that links a base at position 3 or position 4 of the oligonucleotide is a phosphodiester linkage.

69. The compound or oligonucleotide of any one of claims 1-66, wherein nucleoside linkages that link bases at both position 3 and position 4 of the oligonucleotide are phosphodiester linkages.

70. The compound or oligonucleotide of any one of claims 1-66, wherein one or more bases immediately preceding a spacer in the oligonucleotide are linked through phosphodiester bonds.

71. The compound or oligonucleotide of claim 70, wherein only the base immediately preceding the spacer in the oligonucleotide is linked to the spacer through a phosphodiester bond.

72. The compound or oligonucleotide of claim 71, wherein the base immediately preceding the spacer in the oligonucleotide is further linked to a further preceding base through a phosphodiester bond.

73. The compound or oligonucleotide of claim 71, wherein the oligonucleotide comprises a second spacer, wherein a base immediately preceding the second spacer is linked to a further preceding base through a phosphodiester bond.

74. The compound or oligonucleotide of any one of claims 1-66, wherein one or more bases immediately succeeding a spacer in the oligonucleotide are linked through phosphodiester bonds.

75. The compound or oligonucleotide of claim 74, wherein only the base immediately succeeding the spacer in the oligonucleotide is linked to the spacer through a phosphodiester bond.

76. The compound or oligonucleotide of claim 70, wherein two bases immediately preceding the spacer in the oligonucleotide are linked through phosphodiester bonds.

77. The compound or oligonucleotide of any one of claims 1-66, wherein one or more bases immediately preceding a spacer in the oligonucleotide are linked through phosphodiester bonds and wherein one or more bases immediately succeeding the spacer in the oligonucleotide are linked through phosphodiester bonds.

78. The compound or oligonucleotide of claim 77, wherein one base immediately preceding the spacer and one base immediately succeeding the spacer are linked through phosphodiester bonds.

79. The compound or oligonucleotide of claim 77 or 78, wherein the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds.

80. The compound or oligonucleotide of claim 79, wherein one base immediately preceding the second spacer and one base immediately succeeding the second spacer are linked through phosphodiester bonds.

81. The compound or oligonucleotide of any one of claims 1-66, wherein the oligonucleotide comprises a range of bases that are linked through phosphodiester bonds, the range of bases comprising at least two bases.

82. The compound or oligonucleotide of any one of claims 1-66, wherein the oligonucleotide comprises a range of bases that are linked through phosphodiester bonds, the range of bases comprising at least five bases.

83. The compound or oligonucleotide of claim 81 or 82, wherein the oligonucleotide comprises two or more spacers, and wherein the range of bases are positioned between the at least two spacers.

84. A compound comprising an oligonucleotide comprising a nucleobase sequence that shares at least 90% identity to an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292.

85. An oligonucleotide comprising a nucleobase sequence that shares at least 90% identity to an equal length portion of any one of SEQ ID NOs: 1-1264, SEQ ID NOs: 2529-3792, SEQ ID NOs; 5066-5166, or SEQ ID NOs: 5168-5202.

86. The compound of claim 84 or the oligonucleotide of claim 84 or 85, wherein the nucleobase sequence shares at least 95% identity to an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292.

87. The compound of claim 84 or the oligonucleotide of claim 84 or 85, wherein the nucleobase sequence shares at least 100% identity to an equal length portion of any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292.

88. The compound or oligonucleotide of any of claims 67-87, wherein the oligonucleotide is any one of a 19mer, 21mer, 23mer, or 25mer.

89. The compound or oligonucleotide of any one of the above claims, wherein one or more internucleoside linkage of the oligonucleotide is a modified internucleoside linkage.

90. The compound or oligonucleotide of claim 89, wherein the modified internucleoside linkage of the oligonucleotide is a phosphorothioate linkage.

91. The compound or oligonucleotide of claim 89 or 90, wherein all internucleoside linkages of the oligonucleotide are phosphorothioate linkages.

92. The compound or oligonucleotide of claim 90, wherein the phosphorothioate linkage is in one of a Rp configuration or a Sp configuration.

93. The compound or oligonucleotide of any one of the preceding claims, wherein the oligonucleotide comprises at least one modified sugar moiety.

94. The compound or oligonucleotide of claim 93, wherein the modified sugar moiety is one of a 2′-OMe modified sugar moiety, bicyclic sugar moiety, 2′-O-(2-methoxyethyl) (2′-MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, tcDNA, hexitol nucleic acids (HNA), and tricyclic analog (e.g., tcDNA).

95. The compound or oligonucleotide of any one of the above claims, wherein the oligonucleotide exhibits at least a 30%, 40%, 50%, 60%, 70%, 80%, or 90% increase of full length UNC13A protein.

96. The compound or oligonucleotide of any one of the above claims, wherein the oligonucleotide exhibits at least a 100% increase of full length UNC13A protein.

97. The compound or oligonucleotide of any one of the above claims, wherein the oligonucleotide exhibits at least a 200% increase of full length UNC13A protein.

98. The compound or oligonucleotide of any one of the above claims, wherein the oligonucleotide exhibits at least a 300% increase of full length UNC13A protein.

99. The compound or oligonucleotide of any one of the above claims, wherein the oligonucleotide exhibits at least a 400% increase of full length UNC13A protein.

100. The compound or oligonucleotide of any one of claims 95-99, wherein increase of the full length UNC13A protein is measured in comparison to a reduced level of full length UNC13A protein achieved using a TDP43 antisense oligonucleotide.

101. The compound or oligonucleotide of any one of the above claims, wherein the oligonucleotide exhibits at least a 10%, 20%, 30%, 40%, 50%, 60%, 70%, 80%, 90%, or 100% rescue of full length UNC13A protein.

102. The compound or oligonucleotide of any one of the above claims, wherein the oligonucleotide exhibits at least a 50%, 60%, 70%, 80%, or 90% reduction of a mis-spliced UNC13A transcript.

103. A method of treating a neurological disease and/or a neuropathy in a patient in need thereof, the method comprising administering to the patient a compound or an oligonucleotide of any one of claims 1-102.

104. The method of claim 103, wherein the neurological disease selected from the group consisting of amyotrophic lateral sclerosis (ALS), frontotemporal dementia (FTD), ALS with FTD, Alzheimer's disease (AD), Parkinson's disease (PD), Huntington's disease, progressive supranuclear palsy (PSP), brain trauma, spinal cord injury, corticobasal degeneration (CBD), nerve injuries (e.g., brachial plexus injuries), neuropathies (e.g., chemotherapy induced neuropathy), TDP43 proteinopathies (e.g., chronic traumatic encephalopathy, Perry Syndrome, Dementia with Lewy body in association with Alzheimer's disease, Parkinson's disease with or without dementia, Limbic-predominant age-related TDP-43 encephalopathy (LATE)), epilepsy, Cerebral Age-Related TDP-43 With Sclerosis (CARTS), facial onset sensory and motor neuronopathy, Guam Parkinson-dementia complex, multisystem proteinopathy, CTE, and synaptic diseases like autism.

105. The method of claim 104, wherein the neurological disease is ALS.

106. The method of claim 104, wherein the neurological disease is FTD.

107. The method of claim 104, wherein the neurological disease is ALS with FTD.

108. The method of claim 104, wherein the neurological disease is AD.

109. The method of claim 104, wherein the neurological disease is PD.

110. The method of claim 103, wherein the neuropathy is chemotherapy induced neuropathy.

111. A method of restoring axonal outgrowth and/or regeneration of a neuron, the method comprising exposing the neuron to a compound or an oligonucleotide of any one of claims 1-102.

112. A method of increasing, promoting, stabilizing, or maintaining UNC13A expression and/or function in a neuron, the method comprising exposing the cell to a compound or an oligonucleotide of any one of claims 1-102.

113. The method of claim 111 or 112, wherein the neuron is a motor neuron.

114. The method of claim 111 or 112, wherein the neuron is a spinal cord neuron.

115. The method of any one of claims 111-114, wherein the neuron is a neuron of a patient in need of treatment of a neurological disease and/or a neuropathy.

116. The method of claim 115, wherein the neuropathy is chemotherapy induced neuropathy.

117. The method of any one of claims 111-116, wherein the exposing is performed in vivo or ex vivo.

118. The method of any one of claims 111-116, wherein the exposing comprises administering the oligonucleotide to a patient in need thereof.

119. The method of any one of claims 111-118, wherein the oligonucleotide is administered topically, parenterally, intrathecally, intrathalamically, intracisternally, orally, rectally, buccally, sublingually, vaginally, pulmonarily, intratracheally, intranasally, transdermally, intraduodenally, or intracerebroventricularly.

120. The method of claim 119, wherein the oligonucleotide is administered orally.

121. The method of any one of claims 111-119, wherein a therapeutically effective amount of the oligonucleotide is administered intrathecally, intrathalamically or intracisternally.

122. The method of any one of claims 111-121, wherein the patient is a human.

123. A pharmaceutical composition comprising the oligonucleotide of any one of claims 1-102, or a pharmaceutically acceptable salt thereof, and a pharmaceutically acceptable excipient.

124. The pharmaceutical composition of claim 123, wherein the pharmaceutical composition is suitable for topical, intrathecal, intrathalamic, intracisternal, intracerebroventricular, parenteral, oral, pulmonary, intratracheal, intranasal, transdermal, rectal, buccal, sublingual, vaginal, or intraduodenal administration.

125. A method of treating a neurological disease or a neuropathy in a patient in need thereof, the method comprising administering to a patient in need thereof a therapeutically effective amount of a pharmaceutical composition of claim 123 or 124.

126. The method of claim 125, wherein the neurological disease is selected from the group consisting of amyotrophic lateral sclerosis (ALS), frontotemporal dementia (FTD), ALS with FTD, Alzheimer's disease (AD), Parkinson's disease (PD), Parkinson's Disease with dementia, dementia with lewy bodies, synucleinopathies, Huntington's disease, Brachial plexus injuries, peripheral nerve injuries, progressive supranuclear palsy (PSP), brain trauma, spinal cord injury, tuberous sclerosis complex, Pick's Disease, tauopathies, primary age-related tauopathy, Down Syndrome, epilepsy/seizure disorder, depression, traumatic brain injury (TBI), chronic traumatic encephalopathy (CTE), HIV-associated neurocognitive disorders (HAND), multisystem atrophy, amnestic mild cognitive impairment, corticobasal degeneration (CBD) and/or neuropathies such a chemotherapy induced neuropathy, Spinocerebellar ataxia (SCA), SCA type 2, Spinal Muscular Atrophy (SMA), Parkinsonism, Niemann-Pick disease type C (NPC), Charcot-Marie-Tooth Disease (CMT), Mucopolysaccharidosis type II (MPSIIA), Mucolipidosis IV, GM1 gangliosidosis, Sporadic inclusion body myositis (sIBM), Henoch-Schonlein purpura (HSP), Limbic-predominant age-related TDP-43 encephalopathy (LATE)), Cerebral Age-Related TDP-43 With Sclerosis (CARTS), Gaucher's disease, and facial onset sensory and motor neuronopathy, Guam Parkinson-dementia complex, multisystem proteinopathy, Perry disease, and synaptic diseases like autism.

127. The method of claim 126, wherein the neurological disease is ALS.

128. The method of claim 126, wherein the neurological disease is FTD.

129. The method of claim 126, wherein the neurological disease is ALS with FTD.

130. The method of claim 126, wherein the neurological disease is AD.

131. The method of claim 126, wherein the neurological disease is PD.

132. The method of claim 125, wherein the neuropathy is chemotherapy induced neuropathy.

133. The method of any one of claims 125-132, wherein the pharmaceutical composition is administered topically, parenterally, orally, pulmonarily, rectally, buccally, sublingually, vaginally, intratracheally, intranasally, intracisternally, intrathecally, intrathalamically, transdermally, intraduodenally, or intracerebroventricularly.

134. The method of any one of claims 125-132, wherein the pharmaceutical composition is administered intrathecally, intrathalamically, or intracisternally.

135. The method of any one of claims 125-134, wherein a therapeutically effective amount of the oligonucleotide is administered intrathecally, intrathalamically or intracisternally.

136. The method of any one of claims 125-135, wherein the patient is human.

137. A method for treating a neurological disease in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

138. A method for treating amyotrophic lateral sclerosis (ALS) in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

139. A method for treating Alzheimer's Disease (AD) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292, or a pharmaceutically acceptable salt thereof,
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA), optionally, wherein the oligonucleotide further comprises a spacer

140. A method for treating frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

141. A method for treating amyotrophic lateral sclerosis (ALS) with frontotemporal dementia (FTD) in a subject in need thereof, the method comprising administering to the subject an oligonucleotide comprising a segment with at most 7 linked nucleosides, and wherein oligonucleotide shares at least 85% identity with any one of SEQ ID NOs: 1-1264, SEQ ID NOs: 2529-3792, SEQ ID NOs; 5066-5166, SEQ ID NOs: 5168-5202, SEQ ID NOs: 5209-5221, or SEQ ID NOs: 5235-5292, or a pharmaceutically acceptable salt thereof;
wherein at least one (i.e., one or more) nucleoside linkage of the oligonucleotide is independently selected from the group consisting of: a phosphodiester linkage, a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothioate linkage, a thiophosphorodiamidate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or
wherein at least one (i.e., one or more) nucleoside is substituted with a component selected from the group consisting of a 2′-O-(2-methoxyethyl) nucleoside, a 2′-O-methyl nucleoside, a 2′-O—(N-methylacetamide) nucleoside, a 2′-deoxy-2′-fluoro nucleoside, a 2′-fluoro-β-D-arabinonucleoside, a locked nucleic acid (LNA), a tricyclic nucleic acid, constrained methoxyethyl (cMOE), constrained ethyl (cET), and a peptide nucleic acid (PNA)
optionally, wherein the oligonucleotide further comprises a spacer.

142. The method of any one of claims 137-141, wherein nucleoside linkages that link a base at position 3 or position 4 of the oligonucleotide are phosphodiester linkages.

143. The method of claim 142, wherein only one nucleoside linkage that links a base at position 3 or position 4 of the oligonucleotide is a phosphodiester linkage.

144. The method of any one of claims 137-141, wherein nucleoside linkages that link bases at both position 3 and position 4 of the oligonucleotide are phosphodiester linkages.

145. The method of any one of claims 137-141, wherein one or more bases immediately preceding a spacer in the oligonucleotide are linked through phosphodiester bonds.

146. The method of claim 145, wherein only the base immediately preceding the spacer in the oligonucleotide is linked to the spacer through a phosphodiester bond.

147. The method of claim 146, wherein the base immediately preceding the spacer in the oligonucleotide is further linked to a further preceding base through a phosphodiester bond.

148. The method of claim 146, wherein the oligonucleotide comprises a second spacer, wherein a base immediately preceding the second spacer is linked to a further preceding base through a phosphodiester bond.

149. The method of any one of claims 137-141, wherein one or more bases immediately succeeding a spacer in the oligonucleotide are linked through phosphodiester bonds.

150. The method of claim 149, wherein only the base immediately succeeding the spacer in the oligonucleotide is linked to the spacer through a phosphodiester bond.

151. The method of any one of claims 137-141, wherein two bases immediately preceding the spacer in the oligonucleotide are linked through phosphodiester bonds.

152. The method of any one of claims 137-141, wherein one or more bases immediately preceding a spacer in the oligonucleotide are linked through phosphodiester bonds and wherein one or more bases immediately succeeding the spacer in the oligonucleotide are linked through phosphodiester bonds.

153. The method of claim 152, wherein one base immediately preceding the spacer and one base immediately succeeding the spacer are linked through phosphodiester bonds.

154. The method of claim 152 or 153, wherein the oligonucleotide includes a second spacer, and wherein one or more bases immediately preceding the second spacer in the oligonucleotide are linked through phosphodiester bonds and wherein one or more bases immediately succeeding the second spacer in the oligonucleotide are linked through phosphodiester bonds.

155. The method of claim 154, wherein one base immediately preceding the second spacer and one base immediately succeeding the second spacer are linked through phosphodiester bonds.

156. The method of any one of claims 137-141, wherein the oligonucleotide comprises a range of bases that are linked through phosphodiester bonds, the range of bases comprising at least two bases.

157. The method of any one of claims 137-141, wherein the oligonucleotide comprises a range of bases that are linked through phosphodiester bonds, the range of bases comprising at least five bases.

158. The method of claim 156 or 157, wherein the oligonucleotide comprises two or more spacers, and wherein the range of bases are positioned between the at least two spacers.

159. The method of any of claims 142-158, wherein the oligonucleotide is any one of a 19mer, 21mer, 23mer, or 25mer.

160. The method of any one of claims 137-141, wherein at least one (i.e., one or more) internucleoside linkage of the oligonucleotide is a phosphorothioate linkage.

161. The method of any one of claims 137-141, wherein all internucleoside linkages of the oligonucleotide are phosphorothioate linkages.

162. An oligonucleotide and a pharmaceutically acceptable excipient, the oligonucleotide comprising a sequence that is at least 85% complementary to an equal length portion of any one of SEQ ID NOs: 5057-5065 or SEQ ID NOs: 5206-5208, a sequence having 90% identity thereof, or to a 15 to 50 contiguous nucleobase portion thereof, optionally wherein the oligonucleotide comprises a spacer and wherein the oligonucleotide is capable of increasing, restoring, or stabilizing expression of the UNC13A mRNA capable of translation of a functional UNC13A and/or activity and/or function of UNC13A protein in a cell or a human patient of an immune-mediated demyelinating disease, and wherein the level of increase, restoration, or stabilization of expression and/or activity and/or function is sufficient for use of the oligonucleotide as a medicament for the treatment of the immune-mediated demyelinating disease.

163. The method of any one of claims 103-122 or 125-161, the pharmaceutical composition of claim 123 or 124, or the oligonucleotide of any one of claims 1-102 or 162, wherein the oligonucleotide comprises one or more chiral centers and/or double bonds.

164. The method of any one of claims 103-122, 125-161, or 163, the pharmaceutical composition of claim 123, 124, or 163, or the oligonucleotide of any one of claims 1-102 or 162-163, wherein the oligonucleotide exist as stereoisomers selected from geometric isomers, enantiomers, and diastereomers.

165. A method of treating a neurological disease and/or a neuropathy in a patient in need thereof, the method comprising administering to a patient in need thereof a therapeutically effective amount of a pharmaceutical composition of claim 123 or 124, in combination with a second therapeutic agent.

166. The method of claim 165, wherein the second therapeutic agent is selected from Riluzole (Rilutek), PrimeC, Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), pridopidine, dual AON intrathecal administration (e.g., BIIB067, BIIB078, and BIIB105), BIIB100, levodopa/carbidopa, dopaminergic agents (e.g., ropinirole, pramipexole, rotigotine), medroxyprogesterone, KCNQ2/KCNQ3 openers (e.g., retigabine, XEN1101, or QRL-101), bioactive scaffolds, anticonvulsants and psychostimulant agents, a therapy (e.g., selected from breathing care, physical therapy, occupational therapy, speech therapy, nutritional support), deep brain stimulation, levodopa and carbidopa (duopa, rytary, Sinemet, inbrija), istradefylline (nourianz), safinamide (xadago), pramipexole (Mirapex), rotigotine (neupro), ropinirole (requip), amantadine (gocovri, Symmetrel, osmolex), benztropine (Cogentin), trihexyphenidyl (artane), selegiline (eldepryl, zelapar), rasagiline, entacapone (comtan), opicapone (ongentys), tolcapone (tasmar), apomorphine (apokyn, kynmobi), exenatide, lingzhi, BIIB054, BIIB094, Caffeine, sarizotan, embryonic dopamine cell implantation, aducanamab (Aduhlem), memantine (Namenda), Donepezil (Aricept), Rivastigmine (Exelon), Galantamine (razadyne), Namzeric, Suvorexant (belsomra), lecanemab, olanzapine (Zyprexa), quetiapine (Seroquel), SSRIs (citalopram (Cipramil), dapoxetine (Priligy), escitalopram (Cipralex), fluoxetine (Prozac or Oxactin), fluvoxamine (Faverin), paroxetine (Seroxat), sertraline (Lustral), vortioxetine (Brintellix)), divalproex sodium (Depakote), carbamazepine (Tegretol), medroxyprogestrone, Brivaracetam (briviact), cannabidiol (epidiolex), carbamazepine (carbatrol, Tegretol), cenobamate (xcopri), diazepam (valium), lorazepam (Ativan), clonazepam (klonopin), eslicarbazepine (aptiom), ethosuximide (zarontin), felbamate (felbatol), fenfluramine (fintepla), lacosamide (VIMPAT), lamotrigine (Lamictal), levetiracetam (Keppra), oxcarbazepine (oxtellar xr, Trileptal), perampanel (fycompa), phenobarbital, phenytoin (dilantin), pregabalin (lyrica), tiagabine (gabitril), topiramate (topamax), valproate (depakene, depakote), and/or zonisamide (zonegran), for treating said neurologic disease.

167. A method of treating a neurological disease and/or a neuropathy in a patient in need thereof, the method comprising administering to a patient in need thereof a therapeutically effective amount of a pharmaceutical composition of claim 123 or 124, wherein at least one nucleoside linkage of the oligonucleotide is a non-natural linkage, optionally wherein the oligonucleotide comprises a spacer, and wherein the oligonucleotide further comprises a targeting or conjugate moiety selected from cholesterol, lipoic acid, panthothenic acid, polyethylene glycol, and an antibody for crossing the blood brain barrier.

168. The method of any one of claims 103-122, 125-161, or 163-167, wherein the spacer is a nucleoside-replacement group comprising a non-sugar substitute that is incapable of linking to a nucleotide base.

169. The method of claim 168, wherein the spacer is located between positions 10 and 15 of the oligonucleotide.

170. The method of claim 168, wherein the spacer is located between positions 7 and 11 of the oligonucleotide.

171. The method of claim 168 or 170, wherein the oligonucleotide further comprises a second spacer, wherein the second spacer is located between positions 14 and 22 of the oligonucleotide.

172. The method of claim 171, wherein the spacer and the second spacer are separated by at least 5 nucleobases, at least 6 nucleobases, or at least 7 nucleobases in the oligonucleotide.

173. The method of claim 171 or 172, wherein the spacer is located between positions 7 and 9 of the oligonucleotide, and wherein the second spacer is located between positions 15 and 18 of the oligonucleotide.

174. The method of any one of claims 171-173, wherein the spacer is located at position 8 of the oligonucleotide, and wherein the second spacer is located at position 16 of the oligonucleotide.

175. The method of any one of claims 171-174, wherein the oligonucleotide further comprises a third spacer, wherein the third spacer is located between positions 21 and 24 of the oligonucleotide.

176. The method of claim 168, wherein the spacer is located between positions 2 and 5 of the oligonucleotide.

177. The method of claim 176, wherein the oligonucleotide further comprises a second spacer, wherein the second spacer is located between positions 8 and 12 of the oligonucleotide.

178. The method of claim 177, wherein the oligonucleotide further comprises a third spacer, wherein the third spacer is located between positions 18 and 22 of the oligonucleotide.

179. The method of claim 168, wherein the oligonucleotide further comprises a second spacer and a third spacer, wherein the three spacers are located at positions in the oligonucleotide such that each segment of the oligonucleotide has at most 7 linked nucleosides.

180. The method of claim 179, wherein at least two of the three spacers are adjacent to a guanine nucleobase.

181. The method of claim 180, wherein each of the at least two of the three spacers immediately precede a guanine nucleobase.

182. The method of any one of claims 168-181, wherein each of the first, second or third spacers is a nucleoside-replacement group comprising a non-sugar substitute wherein the non-sugar substitute does not contain a ketone, aldehyde, ketal, hemiketal, acetal, hemiacetal, aminal or hemiaminal moiety and is incapable of forming a covalent bond with a nucleotide base.

183. The method of any one of claims 168-181, wherein each of the first, second or third spacers is independently represented by Formula (X), wherein: 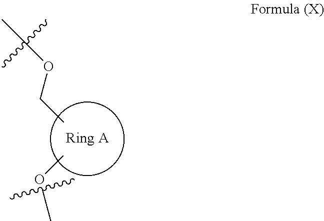
Ring A is an optionally substituted 4-8 member monocyclic cycloalkyl group or a 4-8 member monocyclic heterocyclyl group, wherein the heterocyclyl group contains 1 or 2 heteroatoms selected from O, S and N, provided that A is not capable of forming a covalent bond to a nucleobase; and
the 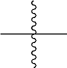
symbol represents the point of connection to an internucleoside linkage.

184. The method of claim 183, wherein each of the first, second or third spacers is independently represented by Formula (Xa), wherein:

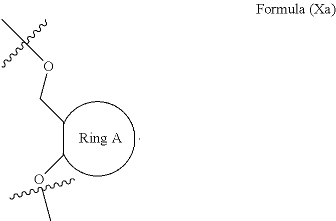

185. The method of claim 183 or 184, wherein ring A is an optionally substituted 4-8 member monocyclic cycloalkyl group selected from cyclobutyl, cyclopentyl, cyclohexyl, cycloheptyl and cyclooctyl; or a 4-8 member monocyclic heterocyclyl group, selected from oxetanyl, tetrahydrofuranyl, tetrahydropyranyl, 1,4-dioxanyl, pyrolidinyl, piperidinyl, piperazinyl, morpholinyl and azepanyl.

186. The method of claim 185, wherein ring A is tetrahydrofuranyl.

187. The method of claim 185, wherein ring A is tetrahydropyranyl.

188. The method of any one of claims 168-181 wherein each of the first, second or third spacers is independently represented by Formula (I), wherein: 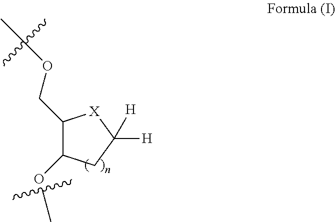
X is selected from —CH₂— and —O—; and
n is 0, 1, 2 or 3.

189. The method of any one of claims 168-181, wherein each of the first, second or third spacers is independently represented by Formula (I′), wherein:

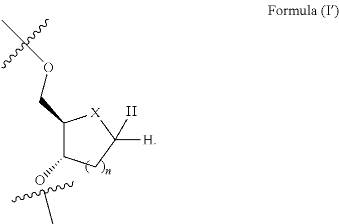

190. The method of any one of claims 168-181, wherein each of the first, second or third spacers is independently represented by Formula (Ia), wherein:

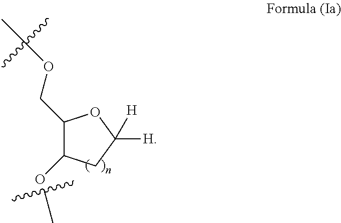

191. The method of any one of claims 168-181, wherein each of the first, second or third spacers is independently represented by Formula (Ia′), wherein:

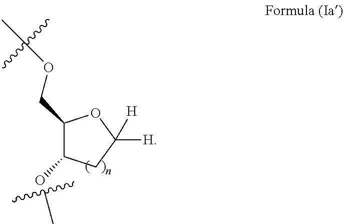

192. The method of any one of claims 168-181, wherein each of the first, second or third spacers is independently represented by Formula II, wherein: 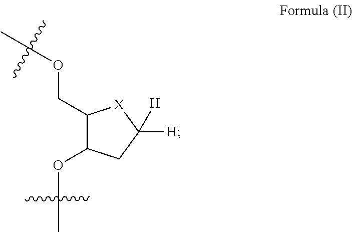
and
X is selected from —CH₂— and —O—.

193. The method of any one of claims 168-181, wherein each of the first, second or third spacers is independently represented by Formula II′, wherein: 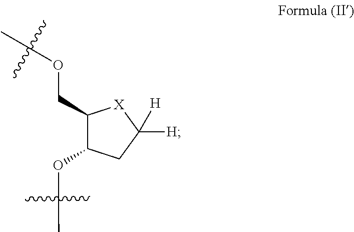
and
X is selected from —CH₂— and —O—.

194. The method of any one of claims 168-181, wherein each of the first, second or third spacers is independently represented by Formula (Iia), wherein:

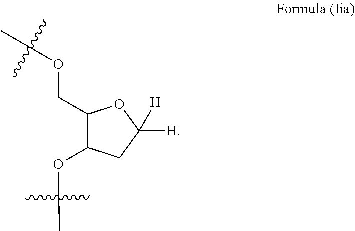

195. The method of any one of claims 168-181, wherein each of the first, second or third spacers is independently represented by Formula (Iia′), wherein:

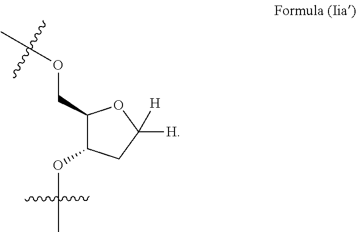

196. The method of any one of claims 168-181, wherein each of the first, second or third spacers is independently represented by Formula (IIi), wherein: 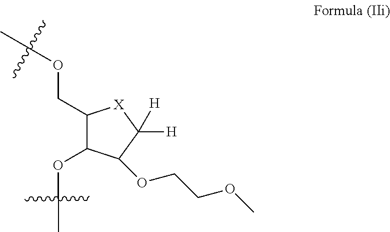
X is selected from —CH₂— and —O—.

197. The method of any one of claims 168-181, wherein each of the first, second or third spacers is independently represented by Formula (IIi′), wherein: 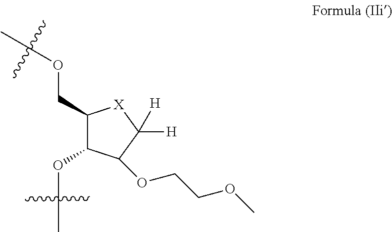
X is selected from —CH₂— and —O—.

198. The method of any one of claims 168-181, wherein each of the first, second or third spacers is independently represented by Formula (IIib), wherein:

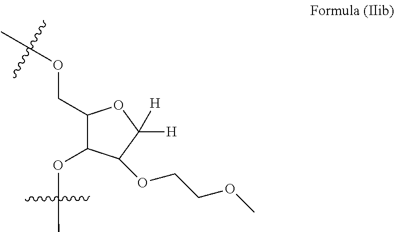 [SMILES: *OCC1OCC(OCCOC)C1O*]

199. The method of any one of claims 168-181, wherein each of the first, second or third spacers is independently represented by Formula (IIib′), wherein: 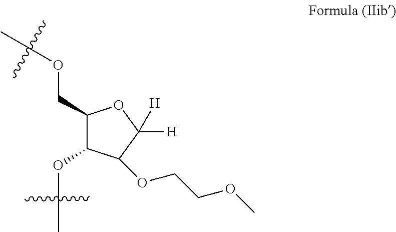 [SMILES: *OC[C@H]1OCC(OCCOC)[C@@H]1O*]
The method of any one of claims 168-181, wherein each of the first, second or third spacers is independently represented by Formula III, wherein: 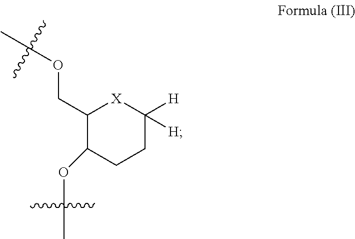
and
X is selected from —CH₂— and —O—.

200. The method of any one of claims 168-181, wherein each of the first, second or third spacers is independently represented by Formula III′, wherein: 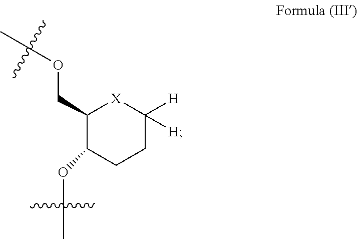
and
X is selected from —CH₂— and —O—.

201. The method of any one of claims 168-181, wherein each of the first, second or third spacers is independently represented by Formula (IIIa), wherein:

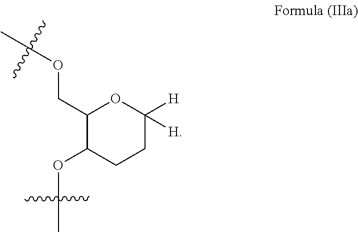

202. The method of any one of claims 168-181, wherein each of the first, second or third spacers is independently represented by Formula (IIIa′), wherein:

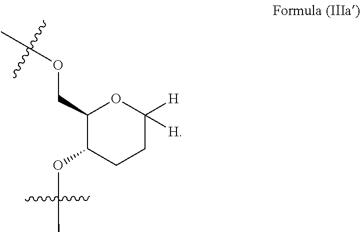

203. The method of any one of claims 168-202, wherein the oligonucleotide comprising the spacer has a GC content of at least 10%.

204. The method of any one of claims 168-203, wherein the oligonucleotide comprising the spacer has a GC content of at least 20%.

205. The method of any one of claims 168-204, wherein the oligonucleotide comprising the spacer has a GC content of at least 25%.

206. The method of any one of claims 168-205, wherein the oligonucleotide comprising the spacer has a GC content of at least 30%.

207. The method of any one of claims 168-206, wherein the oligonucleotide comprising the spacer has a GC content of at least 40%.

208. The method of any one of claims 168-207, wherein the oligonucleotide comprising the spacer has a GC content of at least 50%.

209. The compound or oligonucleotide of any one of the above claims, wherein the oligonucleotide exhibits at least a 30%, 40%, 50%, 60%, 70%, 80%, or 90% reduction of an UNC13A transcript with a cryptic exon.

210. The compound or oligonucleotide of any one of the above claims, wherein the oligonucleotide exhibits at least a 100% reduction of an UNC13A transcript with a cryptic exon.

211. The compound or oligonucleotide of any one of claims 200-201, wherein reduction of an UNC13A transcript with a cryptic exon is measured in comparison to a level of UNC13A transcript with a cryptic exon detected using a TDP43 antisense oligonucleotide.

212. The compound or oligonucleotide of any one of the above claims, wherein the oligonucleotide exhibits at least a 10%, 20%, 30%, 40%, 50%, 60%, 70%, 80%, 90%, or 100% reduction of an UNC13A transcript with a cryptic exon.
